SEQUENCE LISTING (180 sequences)

SEQ ID NO: 1 - DNA, 21 bp, synthetic construct
  source 1..21: mol_type = other DNA
cccgaggcaccgagcgagaat

SEQ ID NO: 2 - DNA, 21 bp, synthetic construct
  source 1..21: mol_type = other DNA
gcagggctcccgaggcaccga

SEQ ID NO: 3 - DNA, 21 bp, synthetic construct
  source 1..21: mol_type = other DNA
agggctcccgaggcaccgagc

SEQ ID NO: 4 - DNA, 21 bp, synthetic construct
  source 1..21: mol_type = other DNA
accgagcgagaatgggaatgg

SEQ ID NO: 5 - DNA, 21 bp, synthetic construct
  source 1..21: mol_type = other DNA
gcaccgagcgagaatgggaat

SEQ ID NO: 6 - DNA, 21 bp, synthetic construct
  source 1..21: mol_type = other DNA
cactgaagcagagggctgcct

SEQ ID NO: 7 - DNA, 21 bp, synthetic construct
  source 1..21: mol_type = other DNA
ttagagcagcaagctgcacag

SEQ ID NO: 8 - DNA, 21 bp, synthetic construct
  source 1..21: mol_type = other DNA
gttagagcagcaagctgcaca

SEQ ID NO: 9 - DNA, 21 bp, synthetic construct
  source 1..21: mol_type = other DNA
gggctgcctttgagtgacgtc

SEQ ID NO: 10 - DNA, 21 bp, synthetic construct
  source 1..21: mol_type = other DNA
agttagagcagcaagctgcac

SEQ ID NO: 11 - DNA, 21 bp, synthetic construct
  source 1..21: mol_type = other DNA
cggcggcggcggcgcgacgat

SEQ ID NO: 12 - DNA, 21 bp, synthetic construct
  source 1..21: mol_type = other DNA
ggcggcgcgacgatgaggatg

SEQ ID NO: 13 - DNA, 21 bp, synthetic construct
  source 1..21: mol_type = other DNA
gccgccgccgccgcctcggcg

SEQ ID NO: 14 - DNA, 21 bp, synthetic construct
  source 1..21: mol_type = other DNA
cgccgccgccgccgcctcggc

SEQ ID NO: 15 - DNA, 21 bp, synthetic construct
  source 1..21: mol_type = other DNA
ggcggcggcggcggcgcgacg

SEQ ID NO: 16 - DNA, 21 bp, synthetic construct
  source 1..21: mol_type = other DNA
ccgccgcctccccgtggaaaa

SEQ ID NO: 17 - DNA, 21 bp, synthetic construct
  source 1..21: mol_type = other DNA
atgggcatgcggcaccgccgc

SEQ ID NO: 18 - DNA, 21 bp, synthetic construct
  source 1..21: mol_type = other DNA
gggcagccagtgggcgccggg

SEQ ID NO: 19 - DNA, 21 bp, synthetic construct
  source 1..21: mol_type = other DNA
gcaccgccgcccgggcagcca

SEQ ID NO: 20 - DNA, 21 bp, synthetic construct
  source 1..21: mol_type = other DNA
agcccggcgcccactggctgc

SEQ ID NO: 21 - DNA, 21 bp, synthetic construct
  source 1..21: mol_type = other DNA
tggagaattacaattcacctg

SEQ ID NO: 22 - DNA, 21 bp, synthetic construct
  source 1..21: mol_type = other DNA
ggtgaattgtaattctccaca

SEQ ID NO: 23 - DNA, 21 bp, synthetic construct
  source 1..21: mol_type = other DNA
ggagaattacaattcacctgg

SEQ ID NO: 24 - DNA, 21 bp, synthetic construct
  source 1..21: mol_type = other DNA
gctggaacccgcgagtgatcc

SEQ ID NO: 25 - DNA, 21 bp, synthetic construct
  source 1..21: mol_type = other DNA
ctggaacccgcgagtgatccc

SEQ ID NO: 26 - DNA, 21 bp, synthetic construct
  source 1..21: mol_type = other DNA
cagtgctggcgctcctcagaa

SEQ ID NO: 27 - DNA, 21 bp, synthetic construct
  source 1..21: mol_type = other DNA
actgggtgcagggagctgcac

SEQ ID NO: 28 - DNA, 21 bp, synthetic construct
  source 1..21: mol_type = other DNA
cccttctgaggagcgccagca

SEQ ID NO: 29 - DNA, 21 bp, synthetic construct
  source 1..21: mol_type = other DNA
acttcctggttcccgcgcgcc

SEQ ID NO: 30 - DNA, 21 bp, synthetic construct
  source 1..21: mol_type = other DNA
tgctggcgctcctcagaaggg

SEQ ID NO: 31 - DNA, 21 bp, synthetic construct
  source 1..21: mol_type = other DNA
caaactctaaaccccaggcgg

SEQ ID NO: 32 - DNA, 21 bp, synthetic construct
  source 1..21: mol_type = other DNA
agccaaactctaaaccccagg

SEQ ID NO: 33 - DNA, 21 bp, synthetic construct
  source 1..21: mol_type = other DNA
aaactctaaaccccaggcgga

SEQ ID NO: 34 - DNA, 21 bp, synthetic construct
  source 1..21: mol_type = other DNA
ccaaactctaaaccccaggcg

SEQ ID NO: 35 - DNA, 21 bp, synthetic construct
  source 1..21: mol_type = other DNA
tggagttggtggggcgggcca

SEQ ID NO: 36 - DNA, 20 bp, synthetic construct
  source 1..20: mol_type = other DNA
tggctggcgagacacgcctc

SEQ ID NO: 37 - DNA, 20 bp, synthetic construct
  source 1..20: mol_type = other DNA
ccgggatcctagctggtggg

SEQ ID NO: 38 - DNA, 20 bp, synthetic construct
  source 1..20: mol_type = other DNA
ggtgggcggagtcctaacac

SEQ ID NO: 39 - DNA, 20 bp, synthetic construct
  source 1..20: mol_type = other DNA
tttaagatgcttctgcagcc

SEQ ID NO: 40 - DNA, 20 bp, synthetic construct
  source 1..20: mol_type = other DNA
cgactgggaagttttgagaa

SEQ ID NO: 41 - DNA, 21 bp, synthetic construct
  source 1..21: mol_type = other DNA
ggagttgggctacccagacct

SEQ ID NO: 42 - DNA, 21 bp, synthetic construct
  source 1..21: mol_type = other DNA
gaggagttgggctacccagac

SEQ ID NO: 43 - DNA, 21 bp, synthetic construct
  source 1..21: mol_type = other DNA
cagcggcagctgaatattgat

SEQ ID NO: 44 - DNA, 21 bp, synthetic construct
  source 1..21: mol_type = other DNA
ctgccgctgagaagattagac

SEQ ID NO: 45 - DNA, 21 bp, synthetic construct
  source 1..21: mol_type = other DNA
gtctaatcttctcagcggcag

SEQ ID NO: 46 - DNA, 20 bp, synthetic construct
  source 1..20: mol_type = other DNA
accgggttctcacctggcgt

SEQ ID NO: 47 - DNA, 20 bp, synthetic construct
  source 1..20: mol_type = other DNA
gcccccacaccataggccga

SEQ ID NO: 48 - DNA, 20 bp, synthetic construct
  source 1..20: mol_type = other DNA
cccgccagagctccagagca

SEQ ID NO: 49 - DNA, 20 bp, synthetic construct
  source 1..20: mol_type = other DNA
ccgcccctactttgctccaa

SEQ ID NO: 50 - DNA, 20 bp, synthetic construct
  source 1..20: mol_type = other DNA
tctcccttcccgtctttcct

SEQ ID NO: 51 - DNA, 21 bp, synthetic construct
  source 1..21: mol_type = other DNA
caccgggttctcacctggcgt

SEQ ID NO: 52 - DNA, 21 bp, synthetic construct
  source 1..21: mol_type = other DNA
caccataggccgatcgggtgg

SEQ ID NO: 53 - DNA, 21 bp, synthetic construct
  source 1..21: mol_type = other DNA
agcataatccttacgccaggt

SEQ ID NO: 54 - DNA, 21 bp, synthetic construct
  source 1..21: mol_type = other DNA
acccgccagagctccagagca

SEQ ID NO: 55 - DNA, 21 bp, synthetic construct
  source 1..21: mol_type = other DNA
cccacaccataggccgatcgg

SEQ ID NO: 56 - DNA, 78 bp, synthetic construct
  source 1..78: mol_type = other DNA
aaaaggccggcggccacgaaaaaggccggccaggcaaaaaagaaaaagggatccgaggccagcggttccggacgggct

SEQ ID NO: 57 - protein, 307 aa, Homo sapiens
MGGLTASDVHPTLGVQLFSAGIAACLADVITFPLDTAKVRLQVQGECPTSSVIRYKGVLGTITAVVKTEGRMKLYSGLPAGLQRQISSASLRIGLYDTVQEFLTAGKETAPSLGSKILAGLTTGGVAVFIGQPTEVVKVRLQAQSHLHGIKPRYTGTYNAYRIIATTEGLTGLWKGTTPNLMRSVIINCTELVTYDLMKEAFVKNNILADDVPCHLVSALIAGFCATAMSSPVDVVKTRFINSPPGQYKSVPNCAMKVFTNEGPTAFFKGLVPSFLRLGSWNVIMFVCFEQLKRELSKSRQTMDCAT

SEQ ID NO: 58 - protein, 798 aa, Homo sapiens
MAWDMCNQDSESVWSDIECAALVGEDQPLCPDLPELDLSELDVNDLDTDSFLGGLKWCSDQSEIISNQYNNEPSNIFEKIDEENEANLLAVLTETLDSLPVDEDGLPSFDALTDGDVTTDNEASPSSMPDGTPPPQEAEEPSLLKKLLLAPANTQLSYNECSGLSTQNHANHNHRIRTNPAIVKTENSWSNKAKSICQQQKPQRRPCSELLKYLTTNDDPPHTKPTENRNSSRDKCTSKKKSHTQSQSQHLQAKPTTLSLPLTPESPNDPKGSPFENKTIERTLSVELSGTAGLTPPTTPPHKANQDNPFRASPKLKSSCKTVVPPPSKKPRYSESSGTQGNNSTKKGPEQSELYAQLSKSSVLTGGHEERKTKRPSLRLFGDHDYCQSINSKTEILINISQELQDSRQLENKDVSSDWQGQICSSTDSDQCYLRETLEASKQVSPCSTRKQLQDQEIRAELNKHFGHPSQAVFDDEADKTGELRDSDFSNEQFSKLPMFINSGLAMDGLFDDSEDESDKLSYPWDGTQSYSLFNVSPSCSSFNSPCRDSVSPPKSLFSQRPQRMRSRSRSFSRHRSCSRSPYSRSRSRSPGSRSSSRSCYYYESSHYRHRTHRNSPLYVRSRSRSPYSRRPRYDSYEEYQHERLKREEYRREYEKRESERAKQRERQRQKAIEERRVIYVGKIRPDTTRTELRDRFEVFGEIEECTVNLRDDGDSYGFITYRYTCDAFAALENGYTLRRSNETDFELYFCGRKQFFKSNYADLDSNSDDFDPASTKSKYDSLDFDSLLKEAQRSLRR

SEQ ID NO: 59 - protein, 1276 aa, Homo sapiens
MRSKARARKLAKSDGDVVNNMYEPNRDLLASHSAEDEAEDSAMSPIPVGPPSPFPTSEDFTPKEGSPYEAPVYIPEDIPIPADFELRESSIPGAGLGVWAKRKMEAGERLGPCVVVPRAAAKETDFGWEQILTDVEVSPQEGCITKISEDLGSEKFCVDANQAGAGSWLKYIRVACSCDDQNLTMCQISEQIYYKVIKDIEPGEELLVHVKEGVYPLGTVPPGLDEEPTFRCDECDELFQSKLDLRRHKKYTCGSVGAALYEGLAEELKPEGLGGGSGQAHECKDCERMFPNKYSLEQHMVIHTEEREYKCDQCPKAFNWKSNLIRHQMSHDSGKRFECENCVKVFTDPSNLQRHIRSQHVGARAHACPDCGKTFATSSGLKQHKHIHSTVKPFICEVCHKSYTQFSNLCRHKRMHADCRTQIKCKDCGQMFSTTSSLNKHRRFCEGKNHYTPGGIFAPGLPLTPSPMMDKAKPSPSLNHASLGFNEYFPSRPHPGSLPFSTAPPTFPALTPGFPGIFPPSLYPRPPLLPPTSLLKSPLNHTQDAKLPSPLGNPALPLVSAVSNSSQGTTAAAGPEEKFESRLEDSCVEKLKTRSSDMSDGSDFEDVNTTTGTDLDTTTGTGSDLDSDVDSDPDKDKGKGKSAEGQPKFGGGLAPPGAPNSVAEVPVFYSQHSFFPPPDEQLLTATGAAGDSIKAIASIAEKYFGPGFMGMQEKKLGSLPYHSAFPFQFLPNFPHSLYPFTDRALAHNLLVKAEPKSPRDALKVGGPSAECPFDLTTKPKDVKPILPMPKGPSAPASGEEQPLDLSIGSRARASQNGGGREPRKNHVYGERKLGAGEGLPQVCPARMPQQPPLHYAKPSPFFMDPIYSRVEKRKVTDPVGALKEKYLRPSPLLFHPQMSAIETMTEKLESFAAMKADSGSSLQPLPHHPFNFRSPPPTLSDPILRKGKERYTCRYCGKIFPRSANLTRHLRTHTGEQPYRCKYCDRSFSISSNLQRHVRNIHNKEKPFKCHLCNRCFGQQTNLDRHLKKHEHENAPVSQHPGVLTNHLGTSASSPTSESDNHALLDEKEDSYFSEIRNFIANSEMNQASTRTEKRADMQIVDGSAQCPGLASEKQEDVEEEDDDDLEEDDEDSLAGKSQDDTVSPAPEPQAAYEDEEDEEPAASLAVGFDHTRRCAEDHEGGLLALEPMPTFGKGLDLRRAAEEAFEVKDVLNSTLDSEALKHTLCRQAKNQAYAMMLSLSEDTPLHTPSQGSLDAWLKVTGATSESGAFHPINHL

SEQ ID NO: 60 - protein, 300 aa, Homo sapiens
MDLPPQLSFGLYVAAFALGFPLNVLAIRGATAHARLRLTPSLVYALNLGCSDLLLTVSLPLKAVEALASGAWPLPASLCPVFAVAHFFPLYAGGGFLAALSAGRYLGAAFPLGYQAFRRPCYSWGVCAAIWALVLCHLGLVFGLEAPGGWLDHSNTSLGINTPVNGSPVCLEAWDPASAGPARFSLSLLLFFLPLAITAFCYVGCLRALARSGLTHRRKLRAAWVAGGALLTLLLCVGPYNASNVASFLYPNLGGSWRKLGLITGAWSVVLNPLVTGYLGRGPGLKTVCAARTQGGKSQK

SEQ ID NO: 61 - protein, 361 aa, Homo sapiens [sequence omitted]

SEQ ID NO: 62 - protein, 655 aa, Homo sapiens
MAEAPQVVEIDPDFEPLPRPRSCTWPLPRPEFSQSNSATSSPAPSGSAAANPDAAAGLPSASAAAVSADFMSNLSLLEESEDFPQAPGSVAAAVAAAAAAAATGGLCGDFQGPEAGCLHPAPPQPPPPGPLSQHPPVPPAAAGPLAGQPRKSSSSRRNAWGNLSYADLITKAIESSAEKRLTLSQIYEWMVKSVPYFKDKGDSNSSAGWKNSIRHNLSLHSKFIRVQNEGTGKSSWWMLNPEGGKSGKSPRRRAASMDNNSKFAKSRSRAAKKKASLQSGQEGAGDSPGSQFSKWPASPGSHSNDDFDNWSTFRPRTSSNASTISGRLSPIMTEQDDLGEGDVHSMVYPPSAAKMASTLPSLSEISNPENMENLLDNLNLLSSPTSLTVSTQSSPGTMMQQTPCYSFAPPNTSLNSPSPNYQKYTYGQSSMSPLPQMPIQTLQDNKSSYGGMSQYNCAPGLLKELLTSDSPPHNDIMTPVDPGVAQPNSRVLGQNVMMGPNSVMSTYGSQASHNKMMNPSSHTHPGHAQQTSAVNGRPLPHTVSTMPHTSGMNRLTQVKTPVQVPLPHPMQMSALGGYSSVSSCNGYGRMGLLHQEKLPSDLDGMFIERLDCDMESIIRNDLMDGDTLDFNFDNVLPNQSFPHSVKTTTHSWVSG

SEQ ID NO: 63 - protein, 492 aa, Homo sapiens
MEPSSKKLTGRLMLAVGGAVLGSLQFGYNTGVINAPQKVIEEFYNQTWVHRYGESILPTTLTTLWSLSVAIFSVGGMIGSFSVGLFVNRFGRRNSMLMMNLLAFVSAVLMGFSKLGKSFEMLILGRFIIGVYCGLTTGFVPMYVGEVSPTALRGALGTLHQLGIVVGILIAQVFGLDSIMGNKDLWPLLLSIIFIPALLQCIVLPFCPESPRFLLINRNEENRAKSVLKKLRGTADVTHDLQEMKEESRQMMREKKVTILELFRSPAYRQPILIAVVLQLSQQLSGINAVFYYSTSIFEKAGVQQPVYATIGSGIVNTAFTVVSLFVVERAGRRTLHLIGLAGMAGCAILMTIALALLEQLPWMSYLSIVAIFGFVAFFEVGPGPIPWFIVAELFSQGPRPAAIAVAGFSNWTSNFIVGMCFQYVEQLCGPYVFIIFTVLLVLFFIFTYFKVPETKGRTFDEIASGFRQGGASQSDKTPEELFHPLGADSQV

SEQ ID NO: 64 - protein, 509 aa, Homo sapiens
MPSGFQQIGSEDGEPPQQRVTGTLVLAVFSAVLGSLQFGYNIGVINAPQKVIEQSYNETWLGRQGPEGPSSIPPGTLTTLWALSVAIFSVGGMISSFLIGIISQWLGRKRAMLVNNVLAVLGGSLMGLANAAASYEMLILGRFLIGAYSGLTSGLVPMYVGEIAPTHLRGALGTLNQLAIVIGILIAQVLGLESLLGTASLWPLLLGLTVLPALLQLVLLPFCPESPRYLYIIQNLEGPARKSLKRLTGWADVSGVLAELKDEKRKLERERPLSLLQLLGSRTHRQPLIIAVVLQLSQQLSGINAVFYYSTSIFETAGVGQPAYATIGAGVVNTVFTLVSVLLVERAGRRTLHLLGLAGMCGCAILMTVALLLLERVPAMSYVSIVAIFGFVAFFEIGPGPIPWFIVAELFSQGPRPAAMAVAGFSNWTSNFIIGMGFQYVAEAMGPYVFLLFAVLLLGFFIFTFLRVPETRGRTFDQISAAFHRTPSLLEQEVKPSTELEYLGPDEND

SEQ ID NO: 65 - protein, 496 aa, Homo sapiens
MGTQKVTPALIFAITVATIGSFQFGYNTGVINAPEKIIKEFINKTLTDKGNAPPSEVLLTSLWSLSVAIFSVGGMIGSFSVGLFVNRFGRRNSMLIVNLLAVTGGCFMGLCKVAKSVEMLILGRLVIGLFCGLCTGFVPMYIGEISPTALRGAFGTLNQLGIVVGILVAQIFGLEFILGSEELWPLLLGFTILPAILQSAALPFCPESPRFLLINRKEEENAKQILQRLWGTQDVSQDIQEMKDESARMSQEKQVTVLELFRVSSYRQPIIISIVLQLSQQLSGINAVFYYSTGIFKDAGVQEPIYATIGAGVVNTIFTVVSLFLVERAGRRTLHMIGLGGMAFCSTLMTVSLLLKDNYNGMSFVCIGAILVFVAFFEIGPGPIPWFIVAELFSQGPRPAAMAVAGCSNWTSNFLVGLLFPSAAHYLGAYVFIIFTGFLITFLAFTFFKVPETRGRTFEDITRAFEGQAHGADRSGKDGVMEMNSIEPAKETTTNV

SEQ ID NO: 66 - protein, 509 aa, Homo sapiens; identical to SEQ ID NO: 64

SEQ ID NO: 67 - protein, 852 aa, Homo sapiens
MAGALAGLAAGLQVPRVAPSPDSDSDTDSEDPSLRRSAGGLLRSQVIHSGHFMVSSPHSDSLPRRRDQEGSVGPSDFGPRSIDPTLTRLFECLSLAYSGKLVSPKWKNFKGLKLLCRDKIRLNNAIWRAWYIQYVKRRKSPVCGFVTPLQGPEADAHRKPEAVVLEGNYWKRRIEVVMREYHKWRIYYKKRLRKPSREDDLLAPKQAEGRWPPPEQWCKQLFSSVVPVLLGDPEEEPGGRQLLDLNCFLSDISDTLFTMTQSGPSPLQLPPEDAYVGNADMIQPDLTPLQPSLDDFMDISDFFTNSRLPQPPMPSNFPEPPSFSPVVDSLFSSGTLGPEVPPASSAMTHLSGHSRLQARNSCPGPLDSSAFLSSDFLLPEDPKPRLPPPPVPPPLLHYPPPAKVPGLEPCPPPPFPPMAPPTALLQEEPLFSPRFPFPTVPPAPGVSPLPAPAAFPPTPQSVPSPAPTPFPIELLPLGYSEPAFGPCFSMPRGKPPAPSPRGQKASPPTLAPATASPPTTAGSNNPCLTQLLTAAKPEQALEPPLVSSTLLRSPGSPQETVPEFPCTFLPPTPAPTPPRPPPGPATLAPSRPLLVPKAERLSPPAPSGSERRLSGDLSSMPGPGTLSVRVSPPQPILSRGRPDSNKTENRRITHISAEQKRRFNIKLGFDTLHGLVSTLSAQPSLKVSKATTLQKTAEYILMLQQERAGLQEEAQQLRDEIEELNAAINLCQQQLPATGVPITHQRFDQMRDMFDDYVRTRTLHNWKFWVFSILIRPLFESFNGMVSTASVHTLRQTSLAWLDQYCSLPALRPTVLNSLRQLGTSTSILTDPGRIPEQATRAVTEGTLGKPL

SEQ ID NO: 68 - protein, 472 aa, Homo sapiens
MGCDRNCGLIAGAVIGAVLAVFGGILMPVGDLLIQKTIKKQVVLEEGTIAFKNWVKTGTEVYRQFWIFDVQNPQEVMMNSSNIQVKQRGPYTYRVRFLAKENVTQDAEDNTVSFLQPNGAIFEPSLSVGTEADNFTVLNLAVAAASHIYQNQFVQMILNSLINKSKSSMFQVRTLRELLWGYRDPFLSLVPYPVTTTVGLFYPYNNTADGVYKVFNGKDNISKVAIIDTYKGKRNLSYWESHCDMINGTDAASFPPFVEKSQVLQFFSSDICRSIYAVFESDVNLKGIPVYRFVLPSKAFASPVENPDNYCFCTEKIISKNCTSYGVLDISKCKEGRPVYISLPHFLYASPDVSEPIDGLNPNEEEHRTYLDIEPITGFTLQFAKRLQVNLLVKPSEKIQVLKNLKRNYIVPILWLNETGTIGDEKANMFRSQVTGKINLLGLIEMILLSVGVVMFVAFMISYCACRSKTIK

SEQ ID NO: 69 - protein, 772 aa, Homo sapiens
MAEAHQAVAFQFTVTPDGVDFRLSREALKHVYLSGINSWKKRLIRIKNGILRGVYPGSPTSWLVVIMATVGSSFCNVDISLGLVSCIQRCLPQGCGPYQTPQTRALLSMAIFSTGVWVTGIFFFRQTLKLLLCYHGWMFEMHGKTSNLTRIWAMCIRLLSSRHPMLYSFQTSLPKLPVPRVSATIQRYLESVRPLLDDEEYYRMELLAKEFQDKTAPRLQKYLVLKSWWASNYVSDWWEEYIYLRGRSPLMVNSNYYVMDLVLIKNTDVQAARLGNIIHAMIMYRRKLDREEIKPVMALGIVPMCSYQMERMFNTTRIPGKDTDVLQHLSDSRHVAVYHKGRFFKLWLYEGARLLKPQDLEMQFQRILDDPSPPQPGEEKLAALTAGGRVEWAQARQAFFSSGKNKAALEAIERAAFFVALDEESYSYDPEDEASLSLYGKALLHGNCYNRWFDKSFTLISFKNGQLGLNAEHAWADAPIIGHLWEFVLGTDSFHLGYTETGHCLGKPNPALAPPTRLQWDIPKQCQAVIESSYQVAKALADDVELYCFQFLPFGKGLIKKCRTSPDAFVQIALQLAHFRDRGKFCLTYEASMTRMFREGRTETVRSCTSESTAFVQAMMEGSHTKADLRDLFQKAAKKHQNMYRLAMTGAGIDRHLFCLYLVSKYLGVSSPFLAEVLSEPWRLSTSQIPQSQIRMFDPEQHPNHLGAGGGFGPVADDGYGVSYMIAGENTIFFHISSKFSSSETNAQRFGNHIRKALLDIADLFQVPKAYS

SEQ ID NO: 70 - protein, 373 aa, Homo sapiens
MGSQVSVESGALHVVIVGGGFGGIAAASQLQALNVPFMLVDMKDSFHHNVAALRASVETGFAKKTFISYSVTFKDNFRQGLVVGIDLKNQMVLLQGGEALPFSHLILATGSTGPFPGKFNEVSSQQAAIQAYEDMVRQVQRSRFIVVVGGGSAGVEMAAEIKTEYPEKEVTLIHSQVALADKELLPSVRQEVKEILLRKGVQLLLSERVSNLEELPLNEYREYIKVQTDKGTEVATNLVILCTGIKINSSAYRKAFESRLASSGALRVNEHLQVEGHSNVYAIGDCADVRTPKMAYLAGLHANIAVANIVNSVKQRPLQAYKPGALTFLLSMGRNDGVGQISGFYVGRLMVRLTKSRDLFVSTSWKTMRQSPP

SEQ ID NO: 71 - protein, 420 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 72 - protein, 1242 aa, Homo sapiens
MASPPESDGFSDVRKVGYLRKPKSMHKRFFVLRAASEAGGPARLEYYENEKKWRHKSSAPKRSIPLESCFNINKRADSKNKHLVALYTRDEHFAIAADSEAEQDSWYQALLQLHNRAKGHHDGAAALGAGGGGGSCSGSSGLGEAGEDLSYGDVPPGPAFKEVWQVILKPKGLGQTKNLIGIYRLCLTSKTISFVKLNSEAAAVVLQLMNIRRCGHSENFFFIEVGRSAVTGPGEFWMQVDDSVVAQNMHETILEAMRAMSDEFRPRSKSQSSSNCSNPISVPLRRHHLNNPPPSQVGLTRRSRTESITATSPASMVGGKPGSFRVRASSDGEGTMSRPASVDGSPVSPSTNRTHAHRHRGSARLHPPLNHSRSIPMPASRCSPSATSPVSLSSSSTSGHGSTSDCLFPRRSSASVSGSPSDGGFISSDEYGSSPCDFRSSFRSVTPDSLGHTPPARGEEELSNYICMGGKGPSTLTAPNGHYILSRGGNGHRCTPGTGLGTSPALAGDEAASAADLDNRFRKRTHSAGTSPTITHQKTPSQSSVASIEEYTEMMPAYPPGGGSGGRLPGHRHSAFVPTRSYPEEGLEMHPLERRGGHHRPDSSTLHTDDGYMPMSPGVAPVPSGRKGSGDYMPMSPKSVSAPQQIINPIRRHPQRVDPNGYMMMSPSGGCSPDIGGGPSSSSSSSNAVPSGTSYGKLWTNGVGGHHSHVLPHPKPPVESSGGKLLPCTGDYMNMSPVGDSNTSSPSDCYYGPEDPQHKPVLSYYSLPRSFKHTQRPGEPEEGARHQHLRLSTSSGRLLYAATADDSSSSTSSDSLGGGYCGARLEPSLPHPHHQVLQPHLPRKVDTAAQTNSRLARPTRLSLGDPKASTLPRAREQQQQQQPLLHPPEPKSPGEYVNIEFGSDQSGYLSGPVAFHSSPSVRCPSQLQPAPREEETGTEEYMKMDLGPGRRAAWQESTGVEMGRLGPAPPGAASICRPTRAVPSSRGDYMTMQMSCPRQSYVDTSPAAPVSYADMRTGIAAEEVSLPRATMAAASSSSAASASPTGPQGAAELAAHSSLLGGPQGPGGMSAFTRVNLSPNRNQSAKVIRADPQGCRRRHSSETFSSTPSATRVGNTVPFGAGAAVGGGGGSSSSSEDVKRHSSASFENVWLRPGELGGAPKEPAKLCGAAGGLENGLNYIDLDLVKDFKQCPQECTPEPQPPPPPPPHQPLGSGESSSTRRSSEDLSAYASISFQKQPEDRQ

SEQ ID NO: 73 - protein, 488 aa, Homo sapiens
MGDRGSSRRRRTGSRPSSHGGGGPAAAEEEVRDAAAGPDVGAAGDAPAPAPNKDGDAGVGSGHWELRCHRLQDSLFSSDSGFSNYRGILNWCVVMLILSNARLFLENLIKYGILVDPIQVVSLFLKDPYSWPAPCLVIAANVFAVAAFQVEKRLAVGALTEQAGLLLHVANLATILCFPAAVVLLVESITPVGSLLALMAHTILFLKLFSYRDVNSWCRRARAKAASAGKKASSAAAPHTVSYPDNLTYRDLYYFLFAPTLCYELNFPRSPRIRKRFLLRRILEMLFFTQLQVGLIQQWMVPTIQNSMKPFKDMDYSRIIERLLKLAVPNHLIWLIFFYWLFHSCLNAVAELMQFGDREFYRDWWNSESVTYFWQNWNIPVHKWCIRHFYKPMLRRGSSKWMARTGVFLASAFFHEYLVSVPLRMFRLWAFTGMMAQIPLAWFVGRFFQGNYGNAAVWLSLIIGQPIAVLMYVHDYYVLNYEAPAAEA

SEQ ID NO: 74 - protein, 388 aa, Homo sapiens
MKTLIAAYSGVLRGERQAEADRSQRSHGGPALSREGSGRWGTGSSILSALQDLFSVTWLNRSKVEKQLQVISVLQWVLSFLVLGVACSAILMYIFCTDCWLIAVLYFTWLVFDWNTPKKGGRRSQWVRNWAVWRYFRDYFPIQLVKTHNLLTTRNYIFGYHPHGIMGLGAFCNFSTEATEVSKKFPGIRPYLATLAGNFRMPVLREYLMSGGICPVSRDTIDYLLSKNGSGNAIIIVVGGAAESLSSMPGKNAVTLRNRKGFVKLALRHGADLVPIYSFGENEVYKQVIFEEGSWGRWVQKKFQKYIGFAPCIFHGRGLFSSDTWGLVPYSKPITTVVGEPITIPKLEHPTQQDIDLYHTMYMEALVKLFDKHKTKFGLPETEVLEVN

SEQ ID NO: 75 - protein, 2346 aa, Homo sapiens
MDEPSPLAQPLELNQHSRFIIGSVSEDNSEDEISNLVKLDLLEEKEGSLSPASVGSDTLSDLGISSLQDGLALHIRSSMSGLHLVKQGRDRKKIDSQRDFTVASPAEFVTRFGGNKVIEKVLIANNGIAAVKCMRSIRRWSYEMFRNERAIRFVVMVTPEDLKANAEYIKMADHYVPVPGGPNNNNYANVELILDIAKRIPVQAVWAGWGHASENPKLPELLLKNGIAFMGPPSQAMWALGDKIASSIVAQTAGIPTLPWSGSGLRVDWQENDFSKRILNVPQELYEKGYVKDVDDGLQAAEEVGYPVMIKASEGGGGKGIRKVNNADDFPNLFRQVQAEVPGSPIFVMRLAKQSRHLEVQILADQYGNAISLFGRDCSVQRRHQKIIEEAPATIATPAVFEHMEQCAVKLAKMVGYVSAGTVEYLYSQDGSFYFLELNPRLQVEHPCTEMVADVNLPAAQLQIAMGIPLYRIKDIRMMYGVSPWGDSPIDFEDSAHVPCPRGHVIAARITSENPDEGFKPSSGTVQELNFRSNKNVWGYFSVAAAGGLHEFADSQFGHCFSWGENREEAISNMVVALKELSIRGDFRTTVEYLIKLLETESFQMNRIDTGWLDRLIAEKVQAERPDTMLGVVCGALHVADVSLRNSVSNFLHSLERGQVLPAHTLLNTVDVELIYEGVKYVLKVTRQSPNSYVVIMNGSCVEVDVHRLSDGGLLLSYDGSSYTTYMKEEVDRYRITIGNKTCVFEKENDPSVMRSPSAGKLIQYIVEDGGHVFAGQCYAEIEVMKMVMTLTAVESGCIHYVKRPGAALDPGCVLAKMQLDNPSKVQQAELHTGSLPRIQSTALRGEKLHRVFHYVLDNLVNVMNGYCLPDPFFSSKVKDWVERLMKTLRDPSLPLLELQDIMTSVSGRIPPNVEKSIKKEMAQYASNITSVLCQFPSQQIANILDSHAATLNRKSEREVFFMNTQSIVQLVQRYRSGIRGHMKAVVMDLLRQYLRVETQFQNGHYDKCVFALREENKSDMNTVLNYIFSHAQVTKKNLLVTMLIDQLCGRDPTLTDELLNILTELTQLSKTTNAKVALRARQVLIASHLPSYELRHNQVESIFLSAIDMYGHQFCIENLQKLILSETSIFDVLPNFFYHSNQVVRMAALEVYVRRAYIAYELNSVQHRQLKDNTCVVEFQFMLPTSHPNRGNIPTLNRMSFSSNLNHYGMTHVASVSDVLLDNSFTPPCQRMGGMVSFRTFEDFVRIFDEVMGCFSDSPPQSPTFPEAGHTSLYDEDKVPRDEPIHILNVAIKTDCDIEDDRLAAMFREFTQQNKATLVDHGIRRLTFLVAQKDFRKQVNYEVDRRFHREFPKFFTFRARDKFEEDRIYRHLEPALAFQLELNRMRNFDLTAIPCANHKMHLYLGAAKVEVGTEVTDYRFFVRAIIRHSDLVTKEASFEYLQNEGERLLLEAMDELEVAFNNTNVRTDCNHIFLNFVPTVIMDPSKIEESVRSMVMRYGSRLWKLRVLQAELKINIRLTPTGKAIPIRLFLTNESGYYLDISLYKEVTDSRTAQIMFQAYGDKQGPLHGMLINTPYVTKDLLQSKRFQAQSLGTTYIYDIPEMFRQSLIKLWESMSTQAFLPSPPLPSDMLTYTELVLDDQGQLVHMNRLPGGNEIGMVAWKMTFKSPEYPEGRDIIVIGNDITYRIGSFGPQEDLLFLRASELARAEGIPRIYVSANSGARIGLAEEIRHMFHVAWVDPEDPYKGYRYLYLTPQDYKRVSALNSVHCEHVEDEGESRYKITDIIGKEEGIGPENLRGSGMIAGESSLAYNEIITISLVTCRAIGIGAYLVRLGQRTIQVENSHLILTGAGALNKVLGREVYTSNNQLGGIQIMHNNGVTHCTVCDDFEGVFTVLHWLSYMPKSVHSSVPLLNSKDPIDRIIEFVPTKTPYDPRWMLAGRPHPTQKGQWLSGFFDYGSFSEIMQPWAQTVVVGRARLGGIPVGVVAVETRTVELSIPADPANLDSEAKIIQQAGQVWFPDSAFKTYQAIKDFNREGLPLMVFANWRGFSGGMKDMYDQVLKFGAYIVDGLRECCQPVLVYIPPQAELRGGSWVVIDSSINPRHMEMYADRESRGSVLEPEGTVEIKFRRKDLVKTMRRVDPVYIHLAERLGTPELSTAERKELENKLKEREEFLIPIYHQVAVQFADLHDTPGRMQEKGVISDILDWKTSRTFFYWRLRRLLLEDLVKKKIHNANPELTDGQIQAMLRRWFVEVEGTVKAYVWDNNKDLAEWLEKQLTEEDGVHSVIEENIKCISRDYVLKQIRSLVQANPEVAMDSIIHMTQHISPTQRAEVIRILSTMDSPST

SEQ ID NO: 76 - protein, 2458 aa, Homo sapiens
MVLLLCLSCLIFSCLTFSWLKIWGKMTDSKPITKSKSEANLIPSQEPFPASDNSGETPQRNGEGHTLPKTPSQAEPASHKGPKDAGRRRNSLPPSHQKPPRNPLSSSDAAPSPELQANGTGTQGLEATDTNGLSSSARPQGQQAGSPSKEDKKQANIKRQLMTNFILGSFDDYSSDEDSVAGSSRESTRKGSRASLGALSLEAYLTTGEAETRVPTMRPSMSGLHLVKRGREHKKLDLHRDFTVASPAEFVTRFGGDRVIEKVLIANNGIAAVKCMRSIRRWAYEMFRNERAIRFVVMVTPEDLKANAEYIKMADHYVPVPGGPNNNNYANVELIVDIAKRIPVQAVWAGWGHASENPKLPELLCKNGVAFLGPPSEAMWALGDKIASTVVAQTLQVPTLPWSGSGLTVEWTEDDLQQGKRISVPEDVYDKGCVKDVDEGLEAAERIGFPLMIKASEGGGGKGIRKAESAEDFPILFRQVQSEIPGSPIFLMKLAQHARHLEVQILADQYGNAVSLFGRDCSIQRRHQKIVEEAPATIAPLAIFEFMEQCAIRLAKTVGYVSAGTVEYLYSQDGSFHFLELNPRLQVEHPCTEMIADVNLPAAQLQIAMGVPLHRLKDIRLLYGESPWGVTPISFETPSNPPLARGHVIAARITSENPDEGFKPSSGTVQELNFRSSKNVWGYFSVAATGGLHEFADSQFGHCFSWGENREEAISNMVVALKELSIRGDFRTTVEYLINLLETESFQNNDIDTGWLDYLIAEKVQAEKPDIMLGVVCGALNVADAMFRTCMTDFLHSLERGQVLPADSLLNLVDVELIYGGVKYILKVARQSLTMFVLIMNGCHIEIDAHRLNDGGLLLSYNGNSYTTYMKEEVDSYRITIGNKTCVFEKENDPTVLRSPSAGKLTQYTVEDGGHVEAGSSYAEMEVMKMIMTLNVQERGRVKYIKRPGAVLEAGCVVARLELDDPSKVHPAEPFTGELPAQQTLPILGEKLHQVFHSVLENLTNVMSGFCLPEPVFSIKLKEWVQKLMMTLRHPSLPLLELQEIMTSVAGRIPAPVEKSVRRVMAQYASNITSVLCQFPSQQIATILDCHAATLQRKADREVFFINTQSIVQLVQRYRSGIRGYMKTVVLDLLRRYLRVEHHFQQAHYDKCVINLREQFKPDMSQVLDCIFSHAQVAKKNQLVIMLIDELCGPDPSLSDELISILNELTQLSKSEHCKVALRARQILIASHLPSYELRHNQVESIFLSAIDMYGHQFCPENLKKLILSETTIFDVLPTFFYHANKVVCMASLEVYVRRGYIAYELNSLQHRQLPDGTCVVEFQFMLPSSHPNRMTVPISITNPDLLRHSTELFMDSGFSPLCQRMGAMVAFRRFEDFTRNFDEVISCFANVPKDTPLFSEARTSLYSEDDCKSLREEPIHILNVSIQCADHLEDEALVPILRTFVQSKKNILVDYGLRRITFLIAQEKEFPKFFTFRARDEFAEDRIYRHLEPALAFQLELNRMRNFDLTAVPCANHKMHLYLGAAKVKEGVEVTDHRFFIRAIIRHSDLITKEASFEYLQNEGERLLLEAMDELEVAFNNTSVRTDCNHIFLNFVPTVIMDPFKIEESVRYMVMRYGSRLWKLRVLQAEVKINIRQTTTGSAVPIRLFITNESGYYLDISLYKEVTDSRSGNIMFHSFGNKQGPQHGMLINTPYVTKDLLQAKRFQAQTLGTTYIYDFPEMFRQALFKLWGSPDKYPKDILTYTELVLDSQGQLVEMNRLPGGNEVGMVAFKMRFKTQEYPEGRDVIVIGNDITFRIGSFGPGEDLLYLRASEMARAEGIPKIYVAANSGARIGMAEEIKHMFHVAWVDPEDPHKGFKYLYLTPQDYTRISSLNSVHCKHIEEGGESRYMITDIIGKDDGLGVENLRGSGMIAGESSLAYEEIVTISLVTCRAIGIGAYLVRLGQRVIQVENSHIILTGASALNKVLGREVYTSNNQLGGVQIMHYNGVSHITVPDDFEGVYTILEWLSYMPKDNHSPVPIITPTDPIDREIEFLPSRAPYDPRWMLAGRPHPTLKGTWQSGFFDHGSFKEIMAPWAQTVVTGRARLGGIPVGVIAVETRTVEVAVPADPANLDSEAKIIQQAGQVWFPDSAYKTAQAVKDFNREKLPLMIFANWRGFSGGMKDMYDQVLKFGAYIVDGLRQYKQPILIYIPPYAELRGGSWVVIDATINPLCIEMYADKESRGGVLEPEGTVEIKFRKKDLIKSMRRIDPAYKKLMEQLGEPDLSDKDRKDLEGRLKAREDLLLPIYHQVAVQFADFHDTPGRMLEKGVISDILEWKTARTFLYWRLRRLLLEDQVKQEILQASGELSHVHIQSMLRRWFVETEGAVKAYLWDNNQVVVQWLEQHWQAGDGPRSTIRENITYLKHDSVLKTIRGLVEENPEVAVDCVIYLSQHISPAERAQVVHLLSTMDSPAST

SEQ ID NO: 77 - protein, 1158 aa, Homo sapiens
MTHGEELGSDVHQDSIVLTYLEGLLMHQAAGGSGTAVDKKSAGHNEEDQNFNISGSAFPTCQSNGPVLNTHTYQGSGMLHLKKARLLQSSEDWNAAKRKRLSDSIMNLNVKKEALLAGMVDSVPKGKQDSTLLASLLQSFSSRLQTVALSQQIRQSLKEQGYALSHDSLKVEKDLRCYGVASSHLKTLLKKSKVKDQKPDTNLPDVTKNLIRDRFAESPHHVGQSGTKVMSEPLSCAARLQAVASMVEKRASPATSPKPSVACSQLALLLSSEAHLQQYSREHALKTQNANQAASERLAAMARLQENGQKDVGSYQLPKGMSSHLNGQARTSSSKLMASKSSATVFQNPMGIIPSSPKNAGYKNSLERNNIKQAANNSLLLHLLKSQTIPKPMNGHSHSERGSIFEESSTPTTIDEYSDNNPSFTDDSSGDESSYSNCVPIDLSCKHRTEKSESDQPVSLDNFTQSLLNTWDPKVPDVDIKEDQDTSKNSKLNSHQKVTLLQLLLGHKNEENVEKNTSPQGVHNDVSKFNTQNYARTSVIESPSTNRTTPVSTPPLLTSSKAGSPINLSQHSLVIKWNSPPYVCSTQSEKLTNTASNHSMDLTKSKDPPGEKPAQNEGAQNSATFSASKLLQNLAQCGMQSSMSVEEQRPSKQLLTGNTDKPIGMIDRLNSPLLSNKTNAVEENKAFSSQPTGPEPGLSGSEIENLLERRTVLQLLLGNPNKGKSEKKEKTPLRDESTQEHSERALSEQILMVKIKSEPCDDLQIPNTNVHLSHDAKSAPFLGMAPAVQRSAPALPVSEDFKSEPVSPQDFSFSKNGLLSRLLRQNQDSYLADDSDRSHRNNEMALLESKNLCMVPKKRKLYTEPLENPFKKMKNNIVDAANNHSAPEVLYGSLLNQEELKFSRNDLEFKYPAGHGSASESEHRSWARESKSFNVLKQLLLSENCVRDLSPHRSNSVADSKKKGHKNNVTNSKPEFSISSLNGLMYSSTQPSSCMDNRTFSYPGVVKTPVSPTFPEHLGCAGSRPESGLLNGCSMPSEKGPIKWVITDAEKNEYEKDSPRLTKTNPILYYMLQKGGNSVTSRETQDKDIWREASSAESVSQVTAKEELLPTAETKASFFNLRSPYNSHMGNNASRPHSANGEVYGLLGSVLTIKKESE

SEQ ID NO: 78 - DNA, 20001 bp, Homo sapiens
  source 1..20001: mol_type = genomic DNA
ccagctgtgtgaccttgggcaagtgatgaatctgctctgtgttcagttttcccatctgccaagggataatggtactaatacccttccagttataatgtatctgaagcacttagaacaatgtctggttcatagttaagccctcaaataaatgctacctaaagtgacaggaattcttagagtgcactggcactcaggaaatatgtttccttttctttttcccctaaacaaactccctgcagcattcgtccactttttctgctctggtctttcctggacctgatgaggaacctcttaagaggaaatatgtcttttcctgtcctttcaactctcttcacaaagtctggctttctttgcttcaaacagaaggctttgaactttgttgcatacacaaatgatgagggaagcttgtggaaagacactctggtctctagcctgaggattcagactcagaggaccatggtagtcctcgtttggatttctgacaagctccatagatgtttctgatatacttccctctttggagaactttaagttaaacatttttctgtagcccacccatattctccagatggagatccaaactgggcacaaaacttccatgactgggtgacctttacaagtatatatctgcatctaaaaaatcgattttgcatacttttttctatgaacaatgagctaatgtttggggaagggagagttcaaatatgaatgttttctcgccaatttgttatgaaaaccactcctctgtgtgttacactttttggaacagagcggagcccaaggcttttctgcccctcccaggaacgctcacggcttactttctttccctgcggtgaggaactcctggaccgtgtcgtagaggccgatcctgagagaggcggagctgatttgccgctgaagccccgcaggcagcccgctgtagagtttcatccgcccttctgtttttaccacagcggtgattgttcccaggacacctttatacctaataacactggacgtcgggcattcaccttggacctggaaataagaaaggtgcagaacagaagggagcgaaattgagttcactcttaagccaagatttccccctgtgttcctattttccgtttcttttctcagcaacattcctcttccatccacctccctctaccgtgtgtgtgtgtgtgtgtgtgtgtgtgtgtgtgtgtgtgtgtgtgtgttcgctcacgcgcagatggagggaggctggctacaatgcacattttgaatatccccctccccaggaactctccagaacccctagggcagtaggataggacagaacccccgaagaatcaaagaaaggaaaaagcacctgactagtaaactcaggagaggccaatattctcagctcttaaaatactaagtgcaccctaaattttgggagaatttggtagtagtcatggtcaacccattctctgtggccctggtggttcttcagctaatcccagaaaaagttccagtttcccatcctaaagcaccagccctgggacaaggccagactgctttggaaggtcacctaccctacctagaaacagtggttacaccaaagcagagaataaatggaatgcacgcaacagcgtcccctctgtcctgagaccccttgtcttacccctctgcctagctacctggagccggactttggccgtgtccagcgggaaggtgatcacgtccgccaagcacgccgctattccagctgagaagagctggacccccagggtcgggtgtacgtccgaggctgtcaggccccccatcttcactcagagactggagatgcagaggaaaagggctccagccccgaaggtggaggaagttcctttcccttgctcttcacgcctgtccgccgggcagcaaacccgatttctgttttttgaaccgaccgccgggcagcggcggtgcagaggcggcggctgcagacggagcgcggtgttgggggccgagtccccgcgtcccctcctacccaccctcgcgccagcaggaccctctgcgttccggtcttccgctgggacttatatagctgggccgctcccgggctgggccgcgcgaggtctcagaagacccgggcgtgggacctgccgggggcgtgtccgggagcaccgggtccctcccattcccattctcgctcggtgcctcgggagccctgcttcgctccccgaaagccctgggccggccgctggttgtctgcgccccgcacaccgcacgtcggagggagggcgcaccgcggcggtcagcacccgcgcccccgcacgtgcctcaccttctgtcaccctttggctgcacaccttcggctggggctttcggaaaccagcacgatatcagagccttggtgaaaggttctgcctagctggagctctcccagaaggaagagcggggcactcttccattcctccaactccccagacatcttgcaggcaccttgcggccttctctgcttccctttcctctcctttaaacttctcatcctccttcctccggattccttctcttcccactaccaaattcagacttggaagaatcccacacaggaaccaagagcactctgaacttgtcaaaaagagcgtttcgtttcatcactttttgagagaagatgtcaaacgagacaatagacacactagaaagcaaatagtacattgctaaagcaccgtacaaaatgagataccacctgttccttctaattagatttcttagagggtgcagtggaaaggacagggcaggttgcttggctccaggtgcagctctccagcttaccaactctgtgaacctgggaaagtcatctgtactccagcttttattttcaacagtgtaaaatgtggttccagagctagcccaggtctgaaatctccctgctaacttacctataatgatgtaatatgtgttggttgtgtggcctttcctagttcgatctttgtgcaaattttattttcaatgtttattcttgtgaaaacttaagaaaacaaaaaacttttaaaaaaaacagtgggaaataataggaaactggagaaagagggaaatggacaaggtcaggtcccagctttcagaacctctcagttaagccaaagatggatttgatattaccagaatccacgtccagctatttagggcctctggtactcaaccctgacttaagccccccaaaagagtcagtggaagtcagagtcatctcctccaggagcaggagaaataagacatgaataaagaataatctccaaaccctctataagtaaaggttgtgtactgtgcaagaagccagtcctaaggcacttccaagggaggtcaggcttgcagagcttccctgaaaaaggcaatgttgacctgacgggtagcctctgaaaaaaaatagaaaggagaaggagccagcgaggtttctcctctgcaaacacttgttgaaataaagatcaactgaggcaaataagtggcatgcatacaggcaaagatgttggggaaagcaagtcgcgtatatagaatgcattctgttggcctgactgactgggaaattgggtgatggggaagaaagagagaggtagggcaagaagttggtgagggatgaggctgtagaagccaaatgagaagcagagcctgcaaatccattatgagtgtagcttcatgttatcacctgtgtagtgctttacagactatgaagtgctctcacataaatgtcagatttaatttgatagacaatacagctttatgaatgattagatgtggagtcaaagattactctatgtttaagaacatgggttgcaagctcagtgacagtaccaatgattgtcatgaggcagttggtaaggggggtgattttaggggtaagaagattaaaccaatttttaaagtcttagaattatttggagagaatactgttgatatccagatagagagcttaggaagctgcatttaacatgctaaaagaggaagaaatagaggaggtatttggtagtcatgatatggatgcaatgaaaggggcatgcttttcacgggaaaatggggcccagctgtgtgaccttgggcaagtgatgaatctgctctgtgttcagttttcccatctgccaagggataatggtactaatacccttccagttataatgtatctgaagcacttagaacaatgtctggttcatagttaagccctcaaataaatgctacctaaagtgacaggaattcttagagtgcactggcactcaggaaatatgtttccttttctttttcccctaaacaaactccctgcagcattcgtccactttttctgctctggtctttcctggacctgatgaggaacctcttaagaggaaatatgtcttttcctgtcctttcaactctcttcacaaagtctggctttctttgcttcaaacagaaggctttgaactttgttgcatacacaaatgatgagggaagcttgtggaaagacactctggtctctagcctgaggattcagactcagaggaccatggtagtcctcgtttggatttctgacaagctccatagatgtttctgatatacttccctctttggagaactttaagttaaacatttttctgtagcccacccatattctccagatggagatccaaactgggcacaaaacttccatgactgggtgacctttacaagtatatatctgcatctaaaaaatcgattttgcatacttttttctatgaacaatgagctaatgtttggggaagggagagttcaaatatgaatgttttctcgccaatttgttatgaaaaccactcctctgtgtgttacactttttggaacagagcggagcccaaggcttttctgcccctcccaggaacgctcacggcttactttctttccctgcggtgaggaactcctggaccgtgtcgtagaggccgatcctgagagaggcggagctgatttgccgctgaagccccgcaggcagcccgctgtagagtttcatccgcccttctgtttttaccacagcggtgattgttcccaggacacctttatacctaataacactggacgtcgggcattcaccttggacctggaaataagaaaggtgcagaacagaagggagcgaaattgagttcactcttaagccaagatttccccctgtgttcctattttccgtttcttttctcagcaacattcctcttccatccacctccctctaccgtgtgtgtgtgtgtgtgtgtgtgtgtgtgtgtgtgtgtgtgtgtgtgttcgctcacgcgcagatggagggaggctggctacaatgcacattttgaatatccccctccccaggaactctccagaacccctagggcagtaggataggacagaacccccgaagaatcaaagaaaggaaaaagcacctgactagtaaactcaggagaggccaatattctcagctcttaaaatactaagtgcaccctaaattttgggagaatttggtagtagtcatggtcaacccattctctgtggccctggtggttcttcagctaatcccagaaaaagttccagtttcccatcctaaagcaccagccctgggacaaggccagactgctttggaaggtcacctaccctacctagaaacagtggttacaccaaagcagagaataaatggaatgcacgcaacagcgtcccctctgtcctgagaccccttgtcttacccctctgcctagctacctggagccggactttggccgtgtccagcgggaaggtgatcacgtccgccaagcacgccgctattccagctgagaagagctggacccccagggtcgggtgtacgtccgaggctgtcaggccccccatcttcactcagagactggagatgcagaggaaaagggctccagccccgaaggtggaggaagttcctttcccttgctcttcacgcctgtccgccgggcagcaaacccgatttctgttttttgaaccgaccgccgggcagcggcggtgcagaggcggcggctgcagacggagcgcggtgttgggggccgagtccccgcgtcccctcctacccaccctcgcgccagcaggaccctctgcgttccggtcttccgctgggacttatatagctgggccgctcccgggctgggccgcgcgaggtctcagaagacccgggcgtgggacctgccgggggcgtgtccgggagcaccgggtccctcccattcccattctcgctcggtgcctcgggagccctgcttcgctccccgaaagccctgggccggccgctggttgtctgcgccccgcacaccgcacgtcggagggagggcgcaccgcggcggtcagcacccgcgcccccgcacgtgcctcaccttctgtcaccctttggctgcacaccttcggctggggctttcggaaaccagcacgatatcagagccttggtgaaaggttctgcctagctggagctctcccagaaggaagagcggggcactcttccattcctccaactccccagacatcttgcaggcaccttgcggccttctctgcttccctttcctctcctttaaacttctcatcctccttcctccggattccttctcttcccactaccaaattcagacttggaagaatcccacacaggaaccaagagcactctgaacttgtcaaaaagagcgtttcgtttcatcactttttgagagaagatgtcaaacgagacaatagacacactagaaagcaaatagtacattgctaaagcaccgtacaaaatgagataccacctgttccttctaattagatttcttagagggtgcagtggaaaggacagggcaggttgcttggctccaggtgcagctctccagcttaccaactctgtgaacctgggaaagtcatctgtactccagcttttattttcaacagtgtaaaatgtggttccagagctagcccaggtctgaaatctccctgctaacttacctataatgatgtaatatgtgttggttgtgtggcctttcctagttcgatctttgtgcaaattttattttcaatgtttattcttgtgaaaacttaagaaaacaaaaaacttttaaaaaaaacagtgggaaataataggaaactggagaaagagggaaatggacaaggtcaggtcccagctttcagaacctctcagttaagccaaagatggatttgatattaccagaatccacgtccagctatttagggcctctggtactcaaccctgacttaagccccccaaaagagtcagtggaagtcagagtcatctcctccaggagcaggagaaataagacatgaataaagaataatctccaaaccctctataagtaaaggttgtgtactgtgcaagaagccagtcctaaggcacttccaagggaggtcaggcttgcagagcttccctgaaaaaggcaatgttgacctgacgggtagcctctgaaaaaaaatagaaaggagaaggagccagcgaggtttctcctctgcaaacacttgttgaaataaagatcaactgaggcaaataagtggcatgcatacaggcaaagatgttggggaaagcaagtcgcgtatatagaatgcattctgttggcctgactgactgggaaattgggtgatggggaagaaagagagaggtagggcaagaagttggtgagggatgaggctgtagaagccaaatgagaagcagagcctgcaaatccattatgagtgtagcttcatgttatcacctgtgtagtgctttacagactatgaagtgctctcacataaatgtcagatttaatttgatagacaatacagctttatgaatgattagatgtggagtcaaagattactctatgtttaagaacatgggttgcaagctcagtgacagtaccaatgattgtcatgaggcagttggtaaggggggtgattttaggggtaagaagattaaaccaatttttaaagtcttagaattatttggagagaatactgttgatatccagatagagagcttaggaagctgcatttaacatgctaaaagaggaagaaatagaggaggtatttggtagtcatgatatggatgcaatgaaaggggcatgcttttcacgggaaaatggggcccagctgtgtgaccttgggcaagtgatgaatctgctctgtgttcagttttcccatctgccaagggataatggtactaatacccttccagttataatgtatctgaagcacttagaacaatgtctggttcatagttaagccctcaaataaatgctacctaaagtgacaggaattcttagagtgcactggcactcaggaaatatgtttccttttctttttcccctaaacaaactccctgcagcattcgtccactttttctgctctggtctttcctggacctgatgaggaacctcttaagaggaaatatgtcttttcctgtcctttcaactctcttcacaaagtctggctttctttgcttcaaacagaaggctttgaactttgttgcatacacaaatgatgagggaagcttgtggaaagacactctggtctctagcctgaggattcagactcagaggaccatggtagtcctcgtttggatttctgacaagctccatagatgtttctgatatacttccctctttggagaactttaagttaaacatttttctgtagcccacccatattctccagatggagatccaaactgggcacaaaacttccatgactgggtgacctttacaagtatatatctgcatctaaaaaatcgattttgcatacttttttctatgaacaatgagctaatgtttggggaagggagagttcaaatatgaatgttttctcgccaatttgttatgaaaaccactcctctgtgtgttacactttttggaacagagcggagcccaaggcttttctgcccctcccaggaacgctcacggcttactttctttccctgcggtgaggaactcctggaccgtgtcgtagaggccgatcctgagagaggcggagctgatttgccgctgaagccccgcaggcagcccgctgtagagtttcatccgcccttctgtttttaccacagcggtgattgttcccaggacacctttatacctaataacactggacgtcgggcattcaccttggacctggaaataagaaaggtgcagaacagaagggagcgaaattgagttcactcttaagccaagatttccccctgtgttcctattttccgtttcttttctcagcaacattcctcttccatccacctccctctaccgtgtgtgtgtgtgtgtgtgtgtgtgtgtgtgtgtgtgtgtgtgtgtgttcgctcacgcgcagatggagggaggctggctacaatgcacattttgaatatccccctccccaggaactctccagaacccctagggcagtaggataggacagaacccccgaagaatcaaagaaaggaaaaagcacctgactagtaaactcaggagaggccaatattctcagctcttaaaatactaagtgcaccctaaattttgggagaatttggtagtagtcatggtcaacccattctctgtggccctggtggttcttcagctaatcccagaaaaagttccagtttcccatcctaaagcaccagccctgggacaaggccagactgctttggaaggtcacctaccctacctagaaacagtggttacaccaaagcagagaataaatggaatgcacgcaacagcgtcccctctgtcctgagaccccttgtcttacccctctgcctagctacctggagccggactttggccgtgtccagcgggaaggtgatcacgtccgccaagcacgccgctattccagctgagaagagctggacccccagggtcgggtgtacgtccgaggctgtcaggccccccatcttcactcagagactggagatgcagaggaaaagggctccagccccgaaggtggaggaagttcctttcccttgctcttcacgcctgtccgccgggcagcaaacccgatttctgttttttgaaccgaccgccgggcagcggcggtgcagaggcggcggctgcagacggagcgcggtgttgggggccgagtccccgcgtcccctcctacccaccctcgcgccagcaggaccctctgcgttccggtcttccgctgggacttatatagctgggccgctcccgggctgggccgcgcgaggtctcagaagacccgggcgtgggacctgccgggggcgtgtccgggagcaccgggtccctcccattcccattctcgctcggtgcctcgggagccctgcttcgctccccgaaagccctgggccggccgctggttgtctgcgccccgcacaccgcacgtcggagggagggcgcaccgcggcggtcagcacccgcgcccccgcacgtgcctcaccttctgtcaccctttggctgcacaccttcggctggggctttcggaaaccagcacgatatcagagccttggtgaaaggttctgcctagctggagctctcccagaaggaagagcggggcactcttccattcctccaactccccagacatcttgcaggcaccttgcggccttctctgcttccctttcctctcctttaaacttctcatcctccttcctccggattccttctcttcccactaccaaattcagacttggaagaatcccacacaggaaccaagagcactctgaacttgtcaaaaagagcgtttcgtttcatcactttttgagagaagatgtcaaacgagacaatagacacactagaaagcaaatagtacattgctaaagcaccgtacaaaatgagataccacctgttccttctaattagatttcttagagggtgcagtggaaaggacagggcaggttgcttggctccaggtgcagctctccagcttaccaactctgtgaacctgggaaagtcatctgtactccagcttttattttcaacagtgtaaaatgtggttccagagctagcccaggtctgaaatctccctgctaacttacctataatgatgtaatatgtgttggttgtgtggcctttcctagttcgatctttgtgcaaattttattttcaatgtttattcttgtgaaaacttaagaaaacaaaaaacttttaaaaaaaacagtgggaaataataggaaactggagaaagagggaaatggacaaggtcaggtcccagctttcagaacctctcagttaagccaaagatggatttgatattaccagaatccacgtccagctatttagggcctctggtactcaaccctgacttaagccccccaaaagagtcagtggaagtcagagtcatctcctccaggagcaggagaaataagacatgaataaagaataatctccaaaccctctataagtaaaggttgtgtactgtgcaagaagccagtcctaaggcacttccaagggaggtcaggcttgcagagcttccctgaaaaaggcaatgttgacctgacgggtagcctctgaaaaaaaatagaaaggagaaggagccagcgaggtttctcctctgcaaacacttgttgaaataaagatcaactgaggcaaataagtggcatgcatacaggcaaagatgttggggaaagcaagtcgcgtatatagaatgcattctgttggcctgactgactgggaaattgggtgatggggaagaaagagagaggtagggcaagaagttggtgagggatgaggctgtagaagccaaatgagaagcagagcctgcaaatccattatgagtgtagcttcatgttatcacctgtgtagtgctttacagactatgaagtgctctcacataaatgtcagatttaatttgatagacaatacagctttatgaatgattagatgtggagtcaaagattactctatgtttaagaacatgggttgcaagctcagtgacagtaccaatgattgtcatgaggcagttggtaaggggggtgattttaggggtaagaagattaaaccaatttttaaagtcttagaattatttggagagaatactgttgatatccagatagagagcttaggaagctgcatttaacatgctaaaagaggaagaaatagaggaggtatttggtagtcatgatatggatgcaatgaaaggggcatgcttttcacgggaaaatggggcccagctgtgtgaccttgggcaagtgatgaatctgctctgtgttcagttttcccatctgccaagggataatggtactaatacccttccagttataatgtatctgaagcacttagaacaatgtctggttcatagttaagccctcaaataaatgctacctaaagtgacaggaattcttagagtgcactggcactcaggaaatatgtttccttttctttttcccctaaacaaactccctgcagcattcgtccactttttctgctctggtctttcctggacctgatgaggaacctcttaagaggaaatatgtcttttcctgtcctttcaactctcttcacaaagtctggctttctttgcttcaaacagaaggctttgaactttgttgcatacacaaatgatgagggaagcttgtggaaagacactctggtctctagcctgaggattcagactcagaggaccatggtagtcctcgtttggatttctgacaagctccatagatgtttctgatatacttccctctttggagaactttaagttaaacatttttctgtagcccacccatattctccagatggagatccaaactgggcacaaaacttccatgactgggtgacctttacaagtatatatctgcatctaaaaaatcgattttgcatacttttttctatgaacaatgagctaatgtttggggaagggagagttcaaatatgaatgttttctcgccaatttgttatgaaaaccactcctctgtgtgttacactttttggaacagagcggagcccaaggcttttctgcccctcccaggaacgctcacggcttactttctttccctgcggtgaggaactcctggaccgtgtcgtagaggccgatcctgagagaggcggagctgatttgccgctgaagccccgcaggcagcccgctgtagagtttcatccgcccttctgtttttaccacagcggtgattgttcccaggacacctttatacctaataacactggacgtcgggcattcaccttggacctggaaataagaaaggtgcagaacagaagggagcgaaattgagttcactcttaagccaagatttccccctgtgttcctattttccgtttcttttctcagcaacattcctcttccatccacctccctctaccgtgtgtgtgtgtgtgtgtgtgtgtgtgtgtgtgtgtgtgtgtgtgtgttcgctcacgcgcagatggagggaggctggctacaatgcacattttgaatatccccctccccaggaactctccagaacccctagggcagtaggataggacagaacccccgaagaatcaaagaaaggaaaaagcacctgactagtaaactcaggagaggccaatattctcagctcttaaaatactaagtgcaccctaaattttgggagaatttggtagtagtcatggtcaacccattctctgtggccctggtggttcttcagctaatcccagaaaaagttccagtttcccatcctaaagcaccagccctgggacaaggccagactgctttggaaggtcacctaccctacctagaaacagtggttacaccaaagcagagaataaatggaatgcacgcaacagcgtcccctctgtcctgagaccccttgtcttacccctctgcctagctacctggagccggactttggccgtgtccagcgggaaggtgatcacgtccgccaagcacgccgctattccagctgagaagagctggacccccagggtcgggtgtacgtccgaggctgtcaggccccccatcttcactcagagactggagatgcagaggaaaagggctccagccccgaaggtggaggaagttcctttcccttgctcttcacgcctgtccgccgggcagcaaacccgatttctgttttttgaaccgaccgccgggcagcggcggtgcagaggcggcggctgcagacggagcgcggtgttgggggccgagtccccgcgtcccctcctacccaccctcgcgccagcaggaccctctgcgttccggtcttccgctgggacttatatagctgggccgctcccgggctgggccgcgcgaggtctcagaagacccgggcgtgggacctgccgggggcgtgtccgggagcaccgggtccctcccattcccattctcgctcggtgcctcgggagccctgcttcgctccccgaaagccctgggccggccgctggttgtctgcgccccgcacaccgcacgtcggagggagggcgcaccgcggcggtcagcacccgcgcccccgcacgtgcctcaccttctgtcaccctttggctgcacaccttcggctggggctttcggaaaccagcacgatatcagagccttggtgaaaggttctgcctagctggagctctcccagaaggaagagcggggcactcttccattcctccaactccccagacatcttgcaggcaccttgcggccttctctgcttccctttcctctcctttaaacttctcatcctccttcctccggattccttctcttcccactaccaaattcagacttggaagaatcccacacaggaaccaagagcactctgaacttgtcaaaaagagcgtttcgtttcatcactttttgagagaagatgtcaaacgagacaatagacacactagaaagcaaatagtacattgctaaagcaccgtacaaaatgagataccacctgttccttctaattagatttcttagagggtgcagtggaaaggacagggcaggttgcttggctccaggtgcagctctccagcttaccaactctgtgaacctgggaaagtcatctgtactccagcttttattttcaacagtgtaaaatgtggttccagagctagcccaggtctgaaatctccctgctaacttacctataatgatgtaatatgtgttggttgtgtggcctttcctagttcgatctttgtgcaaattttattttcaatgtttattcttgtgaaaacttaagaaaacaaaaaacttttaaaaaaaacagtgggaaataataggaaactggagaaagagggaaatggacaaggtcaggtcccagctttcagaacctctcagttaagccaaagatggatttgatattaccagaatccacgtccagctatttagggcctctggtactcaaccctgacttaagccccccaaaagagtcagtggaagtcagagtcatctcctccaggagcaggagaaataagacatgaataaagaataatctccaaaccctctataagtaaaggttgtgtactgtgcaagaagccagtcctaaggcacttccaagggaggtcaggcttgcagagcttccctgaaaaaggcaatgttgacctgacgggtagcctctgaaaaaaaatagaaaggagaaggagccagcgaggtttctcctctgcaaacacttgttgaaataaagatcaactgaggcaaataagtggcatgcatacaggcaaagatgttggggaaagcaagtcgcgtatatagaatgcattctgttggcctgactgactgggaaattgggtgatggggaagaaagagagaggtagggcaagaagttggtgagggatgaggctgtagaagccaaatgagaagcagagcctgcaaatccattatgagtgtagcttcatgttatcacctgtgtagtgctttacagactatgaagtgctctcacataaatgtcagatttaatttgatagacaatacagctttatgaatgattagatgtggagtcaaagattactctatgtttaagaacatgggttgcaagctcagtgacagtaccaatgattgtcatgaggcagttggtaaggggggtgattttaggggtaagaagattaaaccaatttttaaagtcttagaattatttggagagaatactgttgatatccagatagagagcttaggaagctgcatttaacatgctaaaagaggaagaaatagaggaggtatttggtagtcatgatatggatgcaatgaaaggggcatgcttttcacgggaaaatggggcccagctgtgtgaccttgggcaagtgatgaatctgctctgtgttcagttttcccatctgccaagggataatggtactaatacccttccagttataatgtatctgaagcacttagaacaatgtctggttcatagttaagccctcaaataaatgctacctaaagtgacaggaattcttagagtgcactggcactcaggaaatatgtttccttttctttttcccctaaacaaactccctgcagcattcgtccactttttctgctctggtctttcctggacctgatgaggaacctcttaagaggaaatatgtcttttcctgtcctttcaactctcttcacaaagtctggctttctttgcttcaaacagaaggctttgaactttgttgcatacacaaatgatgagggaagcttgtggaaagacactctggtctctagcctgaggattcagactcagaggaccatggtagtcctcgtttggatttctgacaagctccatagatgtttctgatatacttccctctttggagaactttaagttaaacatttttctgtagcccacccatattctccagatggagatccaaactgggcacaaaacttccatgactgggtgacctttacaagtatatatctgcatctaaaaaatcgattttgcatacttttttctatgaacaatgagctaatgtttggggaagggagagttcaaatatgaatgttttctcgccaatttgttatgaaaaccactcctctgtgtgttacactttttggaacagagcggagcccaaggcttttctgcccctcccaggaacgctcacggcttactttctttccctgcggtgaggaactcctggaccgtgtcgtagaggccgatcctgagagaggcggagctgatttgccgctgaagccccgcaggcagcccgctgtagagtttcatccgcccttctgtttttaccacagcggtgattgttcccaggacacctttatacctaataacactggacgtcgggcattcaccttggacctggaaataagaaaggtgcagaacagaagggagcgaaattgagttcactcttaagccaagatttccccctgtgttcctattttccgtttcttttctcagcaacattcctcttccatccacctccctctaccgtgtgtgtgtgtgtgtgtgtgtgtgtgtgtgtgtgtgtgtgtgtgtgttcgctcacgcgcagatggagggaggctggctacaatgcacattttgaatatccccctccccaggaactctccagaacccctagggcagtaggataggacagaacccccgaagaatcaaagaaaggaaaaagcacctgactagtaaactcaggagaggccaatattctcagctcttaaaatactaagtgcaccctaaattttgggagaatttggtagtagtcatggtcaacccattctctgtggccctggtggttcttcagctaatcccagaaaaagttccagtttcccatcctaaagcaccagccctgggacaaggccagactgctttggaaggtcacctaccctacctagaaacagtggttacaccaaagcagagaataaatggaatgcacgcaacagcgtcccctctgtcctgagaccccttgtcttacccctctgcctagctacctggagccggactttggccgtgtccagcgggaaggtgatcacgtccgccaagcacgccgctattccagctgagaagagctggacccccagggtcgggtgtacgtccgaggctgtcaggccccccatcttcactcagagactggagatgcagaggaaaagggctccagccccgaaggtggaggaagttcctttcccttgctcttcacgcctgtccgccgggcagcaaacccgatttctgttttttgaaccgaccgccgggcagcggcggtgcagaggcggcggctgcagacggagcgcggtgttgggggccgagtccccgcgtcccctcctacccaccctcgcgccagcaggaccctctgcgttccggtcttccgctgggacttatatagctgggccgctcccgggctgggccgcgcgaggtctcagaagacccgggcgtgggacctgccgggggcgtgtccgggagcaccgggtccctcccattcccattctcgctcggtgcctcgggagccctgcttcgctccccgaaagccctgggccggccgctggttgtctgcgccccgcacaccgcacgtcggagggagggcgcaccgcggcggtcagcacccgcgcccccgcacgtgcctcaccttctgtcaccctttggctgcacaccttcggctggggctttcggaaaccagcacgatatcagagccttggtgaaaggttctgcctagctggagctctcccagaaggaagagcggggcactcttccattcctccaactccccagacatcttgcaggcaccttgcggccttctctgcttccctttcctctcctttaaacttctcatcctccttcctccggattccttctcttcccactaccaaattcagacttggaagaatcccacacaggaaccaagagcactctgaacttgtcaaaaagagcgtttcgtttcatcactttttgagagaagatgtcaaacgagacaatagacacactagaaagcaaatagtacattgctaaagcaccgtacaaaatgagataccacctgttccttctaattagatttcttagagggtgcagtggaaaggacagggcaggttgcttggctccaggtgcagctctccagcttaccaactctgtgaacctgggaaagtcatctgtactccagcttttattttcaacagtgtaaaatgtggttccagagctagcccaggtctgaaatctccctgctaacttacctataatgatgtaatatgtgttggttgtgtggcctttcctagttcgatctttgtgcaaattttattttcaatgtttattcttgtgaaaacttaagaaaacaaaaaacttttaaaaaaaacagtgggaaataataggaaactggagaaagagggaaatggacaaggtcaggtcccagctttcagaacctctcagttaagccaaagatggatttgatattaccagaatccacgtccagctatttagggcctctggtactcaaccctgacttaagccccccaaaagagtcagtggaagtcagagtcatctcctccaggagcaggagaaataagacatgaataaagaataatctccaaaccctctataagtaaaggttgtgtactgtgcaagaagccagtcctaaggcacttccaagggaggtcaggcttgcagagcttccctgaaaaaggcaatgttgacctgacgggtagcctctgaaaaaaaatagaaaggagaaggagccagcgaggtttctcctctgcaaacacttgttgaaataaagatcaactgaggcaaataagtggcatgcatacaggcaaagatgttggggaaagcaagtcgcgtatatagaatgcattctgttggcctgactgactgggaaattgggtgatggggaagaaagagagaggtagggcaagaagttggtgagggatgaggctgtagaagccaaatgagaagcagagcctgcaaatccattatgagtgtagcttcatgttatcacctgtgtagtgctttacagactatgaagtgctctcacataaatgtcagatttaatttgatagacaatacagctttatgaatgattagatgtggagtcaaagattactctatgtttaagaacatgggttgcaagctcagtgacagtaccaatgattgtcatgaggcagttggtaaggggggtgattttaggggtaagaagattaaaccaatttttaaagtcttagaattatttggagagaatactgttgatatccagatagagagcttaggaagctgcatttaacatgctaaaagaggaagaaatagaggaggtatttggtagtcatgatatggatgcaatgaaaggggcatgcttttcacgggaaaatggggct

SEQ ID NO: 79 - DNA, 4001 bp, Homo sapiens
  source 1..4001: mol_type = genomic DNA
tacgttttaggatgacagcaaacattaatgattcaaaaaatgaaaactgccaacgctacctgctgctcgtaatattttatacaggcggaggtgattcccagagccgctgccatgtaggcaaagcttctagcagctagctgcctatagtgcaacagagaagtgactggccaatctcagccgactggcgactctatccatggctggacaagtagccaagacctttcctccttaagaaaaaaatttaaaggccactattctaaatgcttctgatgtttatttgaaaatgactgcaggaattccaatgattaaaataaatcttccttatttccatatgatctagttcctcaatcaactcctaacagcatcaggaataatacatgatggaaaaatgaaactacagcagctgagacccacagtgccagccagacaaccacccacaggacactccggctactcaaaggcaattaggactttgaagccacttataaccctgactcataatggctacagggcatatgaattatccatatctattccgccttaccaagaaatcagaacaagataacaattacaatccacaaatacgatctaccaaggcaagggagggcaaatggagccaacatttgcaaacccagcaaagagttaagaatcccgggcatgcctcgctagataggaaaaatgcattcacagacggccttggccaaattattttcactttagatgtgtatcgctgcaccacatggacatcttatttaatcaaatcacgtgtgggcttgatcctcccccttaccctacgtgcccccagctacccagaggaaacactcgcagtcttttgccagattcattgttagcagctgggatcctccatgcaattcaattcgagtccatctcaccagagtccacagaaagttttgcttgtttcccaagttgcccaaactttccttccgagagcattccattgtgctgaatgcaaacacgccgaggtcctccgtccccccactagactccagagattacgaggaaactgcttgcgttattttcccccctgtatgaattgtagccagcagagactgtggaattgatgtattgctgtctatttttacactagcagcgagcagctccacacacagtcctgcataaactctattctctctcagcttcaggcagctctccgtggtacaggaagatttaaccattggcttccctgccaccgacgcgaacggaaaaccttgcactgccaggcgtttctttttattcgctggccaaatctttcaaccacgtataggagttttaaaggaagcagcatcgttgttttgtgttctgtgggaaaagcaatctaaaatagcccagcaggattatttaggattttgtaagttctctgtctgatgaacagagagagaggggggaaaaatccggactagagtcaaaacggactatatttgaacacatccattttaatatttttatcttcgtcttattataaatagaaatggcatttttccaaccaggccactaaacaccactggaaagacttcagcttctgattcatatcaatatcttagagcataaatattgcatatgagtagaaacagtgccaaagtcacatggaaagagtttctaactgcaacagatactgatgacagcagctaagctttgatttgctcaagccatctcagagccaccttaaaatagcaaacttattggagttaagataagattgagattcttctaaaagctcctgcctggactactttcctggctcctctctttgcccaagcccatccccccttacaggaataatagcatctgagggaagcgtcagttgtggctgcagcgccgagccctgccccagctcacctcgatgtcactccatacagactcagagtcctggttgcacatgtcccacgccatccagctcctgaatgacgccagtcaagctttttcaactccaatccacagtgacacagagcacacactcatgcaggcaaccagccccttactgagagtgaactgaaggcacctgtcttactacagtccccagtcacatgacaaagctattaaaaagtaggctgggctgtcactcacccagcctcccttcccctgtgcagcttgctgctctaactcgtgacgtcactcaaaggcagccctctgcttcagtgaagtaacgctttaaaaaaaaaaaaaaagaaagaaaaagaaaagaaagaaagaaaggaaacacttagacttttggaggcttcaagcatcatgctgtgattaaagccagctttgaatgccacagactctaaacggagccctggccatataataaccaaaatcccctgcaatctttttttaatttattttccttccaaaacaagcaagtggcaaagctccctgtttcatgacagagtaactatattcttagctaaaacgtgtattccctaagaactctttaattctaagatctccaagtggactcaagctcagtttgggactaaagcaagagcttatcacatgatgcatttttatgagacattttctcattgagggagtgtttgaaagcgcaaacggggctttttttttttttttttttttttttgcttgtttaatcaacaaaatagcctgtatgtgttaaggtataaacaaactcctccacccagaattcggctgcccccatggcagtagggaataccagctcccgaagagttgctgcagttttctttgctccatataaggagaacaagccacaaaaccccatcctttcagcttccttctaattatttccatttctctcataggctcccagaaaacaagtgttagtaaatacagtcgctgctgctctgctcagatcagctttgattcccggctcctatttttcatacatgtagcgtttcctccctaactgccttagggtgcctttttgaataaacattgaattccaaccctagtgccctgggttgtgtagtttgcgttttgaaatcaagaatgtctgtgaactgagggaaaaataccaacacactattagcagaaatattcaattcctgagctttactttctattatttttcagcttgtaccttgagacaatgagggctaatgcaggtaggtgcaactgttttatgatgacactacatattgaatacataacaaactcaaggtactggacacagtggaaaaacacttcctgtaacatgacagtggtctgacttaacccttccaagttcccaggagatgtacacgtgtatgtatctgtgtgaataaacatgttttcataaaatgttaaacccaatatacttttagaatcagctcaattcttttacttgataataatttacctgaaaacaatcccccagttacctaaatataatttgtaaagcattattctactgaggttaatttacactgttcttcattgtccatctccttaaacagaaaaacatggttatttgccctaacacatattttaaatattaaattgctcaaatattaaagtgaaagaaagaacaaaagaaacaaaataccagcagactgctgcttctttcagaccatacatattataagagatttgtgtgctgtgagagctgcaacaagttgctagtaaattcccaaataatgtaaggaagttgacagtttatcattactgggcataaaaaggtggccaagagctttgacattttaagtttgctgaatacagttcaccagcacattttcccttttataaacagcagaaaattactgtagttttaattttctaatgaaagaacaggaaaatacaacctcgggaatgcttttatacaagcaattagtggcattactctgcactccccacaaagaaacactaattttcttcccaattatattctgatttaatgtaaaacttcagtaaattattaaaattcaggacaaaggtcattggctctgccctcaaggatcaactgtgaaataatattggtcacctggccaagaagagtcaatgtttattaattattatcatgtgattcttccattgtgatattcact

SEQ ID NO: 80 - DNA, 14236 bp, Homo sapiens
  source 1..14236: mol_type = genomic DNA
agcaaggactttaaaccttggtcatgtcaggtgaatgttggcaagatttccagaatgtctgcatagcaggattgtaccactttcttgatcaggtgcctgagaacaggacaagaccacaaaccatcaaatcataagctgtcacagcaactgcttttggtcaagttttttcctcctcttccctacctcctccctttctttctcttagcaagcttcaaccaatttacaagtgttcgcttccacaaggacaaatttggaagagtgaagaacaacacaatttatccaaatgtgggatgaaatgaaggaaccaggcaccttgctaaagctagatatcaaaaatttgcataagtagttttgcaaccacagaggtgctatttggaacaaaataaattgcttgtattgtatgcaagggtacaaaccttgaccaactttgcggtgtcttccctgcttgtacatttcttaagcacatccagatattcctactaagaagttcaagttagtatccctcaatagggagacctaaaaagataattgtttgtggacttagaagtcctcaacatctttatttgagaattaagaaacatcagtccaaaatatttgaaatatagaattagttctccaggatgttcttgtaattcttttgtttcttttttgtttatttgcttaatattaggtcatatccctagctctttttttgagatccagcacagaagtcagtgactggaatcctggctctcttctgaaacatttgaattacagtgttttggaatttcctattctaacaaagtttgttatccaaacctgtatctctttctacttttcctgttgggctaaagtgaaggtgttgtcggtctgtctagtaaatccaacctacgagataacataatcacatacattcatttctcagggaccttggcggctcttatctccttgcttttatgatctcttatgaacagcccggaatagcaatttgcagcctgtttgtaaagccatcttagtctgaacccagccttcttctcttttatcacccagcgatattcttcagctaaatcccattcgccacagcaaaatttgctctgagatccattttagaataatataagtattctctgtgtgtatggaacatatcaaacacgtaatggagggtaattatcatttcaatgaactatttattaatagaagatgttttgtgtgctttgcttagatttattgctatgctgcccactgtacattgtagcttgaagctatttagttcacgttcatcagcctctcaggcaagcagctctgctgttcacacagttttggtttttctatgtgcttttttcaactttattttaccattatatcacctagaacattaaacacaatgctcaagttccacgtgttaaatcacaattgctttctactaaagttccaagctacccatttttattaacagttactttattaaatccttaataacacagatagcatagttagttaatttgtgtgttgagccatggatgttaatttgtaaacataataacatactgcaatcatttgcataatagcattttaatctttttagtaaaagctgtaattccctacaaagtacactgcagtagaaactaagtgactattggtaataatacaaaaggccttaaaattctgatcctattatagtgctggagcatacccaaactgcttccataatctctgaaatgttactgttctctgatttaggtgtctgtgtcaagaataaaaatgaagtataactggtagcagtagactatttcctaaatgtaatttagtattcactaagcaccagcattttgcaaatagctctgaggctttccttcaaagttcagagctccagctatgcatattgtgtgaggaggaccctggaactcactctcaactctgtgctgaagagagggctctgataaatcatgctgatttggcgcagggaagggcatcggccaccagggatttgactactgagtctccttcctcctctctgccctaccagcctgattgctcctccttgaactgacggtttctgagcagaagttcagaaaagaatgaaaaagtgcagtgaagctttaaaaaagctagtttaaaaaaactcaatgggggctcactccctctacagcccattaacacccattgtaagactgctccccaccctaacccccaaaaaaagaaaaggagaaagaaaaaaaaaagtagttgtttaagtcaggccaagtcattttatgcaagatctggtaattcattaatccatctggctgcaaaccttgaatgaataacttgcagtagtaatggagatgggcaggtctacaaaatattagtgtttagaatctgaaactgtgcaaatcactccatcattttggaaaagaaaaggcacaaaagtatcttctatcatcactttgggacaaatggcttaatggataagaagtcatttagcttcaaacaactctgccattgccaagaataagagtgtgtatttcattgtggatctctctccccttttccctgctctcattctagctacagtaaagcataggatgctgccttgctttcaaacgtacctctgagcgcacagtcttaaaagttaaaaggaggccgggtgcagtggctcacgcctgtaatcccagcattttgggaggccgaggcaggcgaatcacgaggtcaggagattgagaccatcctggctaacacggggaaaccccgtctctactaaaaatacaaaaaaaaaaaaaaaaaaaaaaaaaaaattagttgggcgttgtagcagtcgcctgtagtcccagctactagggaggctgaggcaggagaatggcgtgaacccaggagggggaggttgcagtgagctgagatcttgccattgcactccagcctgggcgacagagcgagactccatctcaaaaaaaaaaaaaaaaaaaaaaaaaaagttaaaaggaacctgagaggttaagaagaagaaaaaaaaatggcccttactgaaattgttttacatcacaggcaattcattttataaaacccacaataaaatttttcattactcaaagaataggcacataattatagggacaaattaaccttgttagatgtgaagtctgggatagggaatggtggaggaacaatttggctctctgctttaacaatgtggtctttaatgttcagctttttatctgctttgtgtgttgattccatccagaggtgagtcatctgctgggctgtgcttccaggacctggcgtagagatagtggggcatggacaaataaacacagcaagtgtataaggtctccacacagacacatggttgctatacaaatagacagtttgtccaatgcagcaccctgaaccttgtgattactttcacctacactattatctttgaagtaaatctaagaagcaatgagccactttttcctgattctgctccaagatttcctccttgtgaatttgggtctccactattttggccccaaagatatccaggcagagtaactttgatgagccagttgagactccgggaggttggaaaaacctacaataagcccataaaatgtcccagtttactttctggagtgcctagagtatctgacatgaaatcattggtttctcctatcccaaaatacaaaatagcactttctgaaatccttgtgcctaatgaaacatacacattctcactttgcaaactgcaaatggctaagaaccgaatcaacgtccataaaaaaaaaaaaaaaaagcagccaatttcatctctcctagccacaccttccattaacaaaagccaccagcaggaaagttagtgccagtgaaagatggtcacgtaacctgcatcctgtgagttgctatttattagcaggggttgataccaagagacagagccctgtgggtcagaactcctgagccatatttttccagtggtcagcataatggcagggaagaaatagggtgataaaccagatgttcccaagataagccagatgttatcaagccgagaagtctaaaaccacagtgcttaaatgaggtgtgtagatgaacacacacacacacaaacgaacaacactatttagtctccctgcaagataagccgacaggagaggctgaaaataaagcagcacacaggctgggtcagattgttgcttcctgatttatgactctccaagatgaacatctaactgatttatacccagaatttggaattttgtggtagactacaaatgcaaagataagtacaacataaatttttaaaatactccataagtctccatgagtttcaacattcttcaatgttcttatgttacagccaattatctctccacttaaatggctattattttcttacttgaagtctatgttctctgtaagtgtcattaccaagtgtggcaaaaatctggctgtgggccgtaaatttaatttaaatgacaaacatttattgattgcaaattggaaatcccagaaatcttataaaagtgattattgtaaattaattcagtcctcccacataacaaaaaagaatttattcttccaatgattaaattaaggtgaggttgtatatacatgcattctttttttcttgtctatttgttttgttaaaattccaggaggtttgccattcatagaaaagtctgtctgaccacaggcacagtcagaagcaggaattcattaccatggaaggctgcaggatttcctccaaggagacaagtgaaaaaaagcagacaccacgtaaatttgtggattgggttaaatatgttctgtccctgaggcagtggaaagatagagggcccctttgtaaaatgactcacgagtctaggaaagtacttcagctcctaaaggtgagcagaaacttaaccaggaacgatatccacgaggttctgcttctctttggaatatctttgagttagcattccttaccacctctctctctatccgaactcagagttatcagggctgatggaattttccttcaaggtactttgaatctgttccctgaatgccaccccctctgctatcaccttgtcccttccctgtacctgtatcacattgggtttggaatgcagcactgcctgctgtctgattttccgtatttcctacttgcctacacctcttacgactgtcagcattatcactcttaaataccacctccattatgttgatctgctaaaaaatcttcaatgacatcctactttctaaacagactctaaacatttccaactgggttctcatgacccaccgtaattttgtacctaccttacatccaaacttactcccactactgccacgttcccattaatatgtattttactttgagtaaaccaagctcacctccacatcccgtctatgaaaacgctctgctcttctcttacaaggctttgtgaatttattcttgctgtttatctcctcagctagaaatacacatacatacacacacatatatatgcatgcatatatgcatacacgagctcgcaatgtttgcaaattgcaaatatatatataatcctatgtaggtacatacacatatatacatatacatgaatatacagtcttgtattgttttcgaattgctaagaactaaaaacatatgcatatacttgtatatttgtatacacagccttgcatggtttcctaattgttttacatttgttcattttgcctcccaaactagatttaaagtcctttaggacacaggccttcttttgtgctttgtatataatcttcacattacctaacgcagttcattctatcaataaacacatgaagatcaactcactcattatgcaacaagtatttgttgagttcttactttctgctattttctgagaaaatatctagaaaaatgtcagtgaacaagacaggcagattccctactcctcaaatgctcatggtctgttgaactgccaatatttgttatttttaactttttgataggtacataatatatgtccatttttgcaaaatttagaatataactgaaagccaaatgaagaaaagtaaagtatctatattcccactagaataaccaagtagccttgaaagcgtccacattcatttccttgattgcatatagcaggataatttacacacaaatacaaaaccaccactcacccttttctgctacaaagttcacagtttcaggcttcctgagagttccagcaatgaccaaacatgaacgctggctacagctgtgagagtccttctccccccatcagcaaaggcagtcctcagaacgtattacaagtaatgacctagagacgactgcattctcacttgccatcattctaacgaaccccaagtagcttcattcagaagtaacagacgaatgaatgaaagatgcccattcagttgtatcatggaggcattaaagcaggtgttaaaataaaccagggacaaggttgcccacagtgacccagaaaggccaatatgctatttaaactaaccccatgccatccatctttacagactgctgtgctgcaaaagtagtcctttattctcctccttcaaacaatgcacaaaggcaacagagggaaggaagttatgttgcaatattctgaaacataaatcttgctaccagccggtccagggacgcatcctgttttccttacacatggatcagattcactggaggctgttacctctgagccttttgctttgttttttaaagtggcttctgaatcgtacctggtcacctgcatttacacatccaaacacaggttgcgtaagcagagaacattgtttgcgtgtccagttactcaaccaacatgtgctattgcatgttgctacccctctgccaccacccagaattccctttctcaaattgtgacattcagcttcattttttatcattattaagtttgtgatgttaaataaaaattaaacttagattcaaacatatggaagaaacaaaaagaagagatacataatttactctcaacattttaagactctgccactctattatatatccctaaatcccactttctgctttgatgagaattcagtcgatggccagtttccagtggtgtcattgacgtgctacttgagtcagggaaaagcatttaactactcagtgcctgattttattctgggaatccaaagtagataatgcttgttattgtcagatcagagacagcagagcccgtgactgtttaacttactcaagtacaaggccttctcaaatgtgatgaggcagggctttgcaaattggaagaaggtgagtgcaggtgaaaagtccactgtgagtggccggccctgggaaggcacttccctgaaatgctgctttggtagaagcaacacaaacctgtggccatgaaagagttatctggtttcatacgaaccttcttcacagggaagacccaattttagaatgtcacctgtgctgaaaacacgctttcctttcacattacagaaaagatgcctgagtttaacactagagggcaaagagtggaggtgtcctcatgccatgcaaatactcctgaagctgctgctgttggtaagggcattgccacatatgactttaccctttgcctcgataaagaatgggctttgtgacatcagagaaaactgttatgggaacttacctaaacagtcacttttgccctctgacactaagaggatacctgacatattagtcctggagccccaggcatcccgatatcgttatacgacttctcattctttatatccagtagcttactatgtcttctctcagaaatctgaccaattaatcattagtaatttataaaggggaagcctgtgactttatttaaaagcagggtttttcaacctcagtgcttttttcattggaggctgaatcattttttgtggtgggagctgtcctgtaggctgttggatgtttagcagcattccttgtctctagtctctactcaataaatgctcgtagcatccccttgtccccagggataacaaccaaacatgcctccagatattgtcaaatgtcccgtgaattggggtgtggggcggggaccaaaatcgcctttggttgagaatcattggtttaaaatgacctaatgagagactaaattaaatgaacttctgttttagagtgctgaaaataattttattagcatagaatatgatacagggagacaaaaagggtggcatggcatatattaagcacctacatgtagataattaactagatgctgaaccaagtctcctgaaggtataaatgcaaagtacctctcacaaataccacaacacaaaatatagggacataaaagttatggagaaagttgtattttttttaaagtagcccaccagagaggcattgactcctatgccatctcccctaccgctaagttattctctagcctctacaaaatgcatggcattcacctgatatgaaataggtgatttttagataagaggtctatggagatggacattttttaaagcattgtttatttaatgcagcatgacttagaatgggcctgtaacttgacaagtgtactgagtgaggaatgttcaatttaccagttctaactggccaagtcctcacatgaactttctaagacccggctgagaagcctgggaaagtagaactaaccacaaccttcagtgttccctccaccttgttctgtctccccagtccaaactctactaccttaagcattcttctaaaacccacttaatctccagaggtattacaaaatctcagccataggcagaacctacagccgtctcggctcaccagctaactctacctccgtgccaaccaacactgaaaggaaagattctgttctatttttaaatatattgagcaaagaagcagcttgatatgtctttgtaaaggatggctgtcacccaaattcatgggttctctatttttatcaaggatataaactttgaagtccgataaatttggggttcaaatcccaggtctttattgtcaactgtgtgatttggggaaaacaaataacctctctgagtttcagtgtctgcatatgtaaaacaggaatgataacaataggacctaacaataggttgttttcaaggataagtgagatgacatatattaagcatgagtataccaaaatgttggccattattgtcattactattattgttatcaacattaatatattcatcattctctcatgatttaacaaacatttattcagcatgtattatatgctgggttccagcaaaactcagttaatattttaaacttggctttcttaagactcagatttcaaccaaataacatggaagtataagaaatgtctttgctgatgaataagaaaagtccagcacatagtaggtgctcagaaaacattagatgatgatgatgatggcactggcggttgtgataaagaaaactagccgatatgtcatccttcgtttaattcacttgccagagtactcactaaatgtttgttcctataaaaaataatgtatttcaagtaaatgagtactgatcttgtgggaaagaattccttttctcaaagtaacactttaataggaatagattaacattctttattatagggtaggcttataataagagtccggagtcacggaggtaattcttgctactgaaaagtaaatgaattttgactgatttataagtatacagtagattagtcaagtttagcaattcccattatataaagaaattagagatatgaaccaaaaatataattaataataataatcaaaagagctcacctaattagtaaagcaaacaaaggtagacatctacccccttggttaaaaagaaaggccttcttatttattaatttatatgaggtaccatttattgctcattctttctgtgggaaatagacaggaataaacaggtttagacttttgttcttctttgtcactaatctttcacaaccctttagtatttcaacacagtttaactcactcacagttgttgattcctagtccctagttgatttattttttaaatctttaaaaaaataaaaacaaaacatttgttttccaaatgctgtttttccataaattagaaagtatgtaactgtgatgaaaagtcttttgatgatagcaaagtaagaactcattatgcattcaggtctattaccatgttagtcgggcccaagccaaactcaatgaaaaattaatgtttccaaaggatgtccttacctcaaatatgtttgaaggctcattgttgtactgattggatattatttctgattggtcactgcaccacttgagtccacccagaaagctgtctgtatccaagtcgttcacatctagttcagaaagatcaagttcaggaagatctgggcaaagaggctggtcttcaccaaccagagcagcacactgcaggaggcagaaaaaaaaaatttaaaaaagcttccattaaccacaggaatttattaaactgcaatagcatgtgataggaattaagcctagttaattcaactacactaccaaagctagtttctggcactttaatcaccaggaaaaacatcctaaaatctttactcacttggctcctgaaggataatttccctgctttattccatccaaaagctacctccaaacacatggtaaataccaagctccacgaagctttctctgattcttccagttagaaatcaactagatatttttggacctctttgtacaaaatccttgatacctgtttgatagttactgcttccaaagttttaaactgtcttatagttaaccataatacaaacttgattttaccttggaaattttatcacacataatagacactcaataaactattgctaaatggaaaaagaacagaggaactacactaggaaaaattttctccatttacactccttttaagcattataatccaaataattcatttagcaactaatgatgcaaagccctttgactcatctgtggttctcctgaattgttatttaaatcatttactcttatttgactcttcatgactttctggacttcatccttaaaggcaaaagcaagttccttaagatgaaggacaaaaatcataattttttttctatacggcttttgtatgtaacttgcaagatgtgtgtgtgtgtgtgtatgtgtttgcacacgtatgtgtgtgtacggttggaatcacctagtacctggaaaagaaaactcacaatagcataccatcactatattttgttcattatttatgtatatctaaactgtgctctacgtgaattcaaaataactatttttgtttagagttttaaaataatttctacatattgagaggtttaaaatatctctaccatggctgggctgcttatcaccaagattattttagattgtcaaagcatttcatccccttacatgaaatagacagtctttttaactgttttaccatgcggcttttggaggcaagtggactccccttaccaatgaggtggtctcctaaacttgaattggctacctaaccgctaatgtcagtccgcagacagtaaagaacactgcaggaactcctattgcaggggtctggcattgccagaacagtaaggacttcatccacctgtaagtgggggtgtctaaatcaggaggcacacactgacgcagcaccctcatggccctgccttccaaggtcaggagagatgctgtggaattgttcacatccaggctgcagttaggcataaaatgaaagatgctccttgacatttacattttgaatgaaataagttttatgcagattttctttctcaagcacccctgttatccctggcagggattcctagcaggccacattacacaggtcatatgcagcagcgtgatcaaaagcaacacgctgagaagtaagtactgggtaatggagtcagcagttggtaaggggaggatggggggagactgaaaatcattgccatccttctctaagaccattggaccccagtgagatccctggcagcagggacaagggaatgcaaagaaagggccctcctcaccatcaaaactctttccttatcattttattggtctttccttttattatctgagttagcccaaaattacagatcagtatacttgacaaggcatataagtaactggaggctgggctgaagagatctcgttctggtacaatgctcatatttggttgtggatgtaacactgcccagactccattcttgcccacacagctgatcctccaagcccaaacacactatcttaacttttcctcaatgtcaccaattcagagatgggggagaactttttcaacggtgcaagatacactaggattttataggggcatagtctttaaaaaaaaaaaaaaaaaacatgaacaacaaaaatagcaacacaaaagtaaaaataatcaagaaaatgtaggctgtctacatatggcttaagttcctgttgtgaaacttaggaaccaaccatgttttggaatgctaaagtagcatactgatttgtggacaacttcttcctgtccctctctttaactgccctctttggcatgacttgctcataaccctttaaccttccataaatatgtatttttaaataacttttgaaaaccattgaaaagaccaggtcaatttgttgtattggtgctatcttgtcatttacatattcctttccttcctctcacttgttcgctcgtgcgctctctctctctctctcattttgcttttatgttatggttgagaaaagtgcatgtgttcttgccaatgttgacatgctaataaaatctgtgcttctgaatgtggcattatcctgctaatctaaaacacattagtggtagcaagtgttcctgttattaatttatatttggcctttggacaatgccagacttaacagtccaagccctgtaactgtcctcatctgacatgctgatatttactgtttccctatacctctgtaacaggtgtgctttattgcaattacacagacacattctactgcctctttgcaactatgacagctatttcacaactgtagcctcaattttataataagatttaagaggataaagcagtcactaaacacaaaaggctaattattgtatgttatttcaccccaacagacttttctcttagttaaggagcagacaataaattagcttaactgcactgatgataatgaatcattataattaagtataattctccattaaccttttggaggtttttgtgccattgatggtaaattttaaagagagagagagaagaaggagtggacttggccacattaattatattaatgaaaaaagaaactgggttcttaaaaaaattagttaagacaggggaaagggggtggagagaggggcagtaaagaatgacagtcttagtggggtgtatgtcatgtgaattgtctctatcaatcttagtaaccttactgtatatcaccttcattaaactgtgaaacacggggcactccaagtagctatagtgatataggcctatattaaccaatgtactaccctgtcattatgtgcaaaagggcataatacgttttaggatgacagcaaacattaatgattcaaaaaatgaaaactgccaacgctacctgctgctcgtaatattttatacaggcggaggtgattcccagagccgctgccatgtaggcaaagcttctagcagctagctgcctatagtgcaacagagaagtgactggccaatctcagccgactggcgactctatccatggctggacaagtagccaagacctttcctccttaagaaaaaaatttaaaggccactattctaaatgcttctgatgtttatttgaaaatgactgcaggaattccaatgattaaaataaatcttccttatttccatatgatctagttcctcaatcaactcctaacagcatcaggaataatacatgatggaaaaatgaaactacagcagctgagacccacagtgccagccagacaaccacccacaggacactccggctactcaaaggcaattaggactttgaagccacttataaccctgactcataatggctacagggcatatgaattatccatatctattccgccttaccaagaaatcagaacaagataacaattacaatccacaaatacgatctaccaaggcaagggagggcaaatggagccaacatttgcaaacccagcaaagagttaagaatcccgggcatgcctcgctagataggaaaaatgcattcacagacggccttggccaaattattttcactttagatgtgtatcgctgcaccacatggacatcttatttaatcaaatcacgtgtgggcttgatcctcccccttaccctacgtgcccccagctacccagaggaaacactcg

SEQ ID NO: 81 - DNA, 4001 bp, Homo sapiens
  source 1..4001: mol_type = genomic DNA
gttctctgccccaacccctgtagaaagatggtcagggtgtggggagggtgggtaaaggggtcgatagaccctcgaaaggacggcagggaatggggctgtgccattgctcatggcccttctctaggcgacactcacctggtgccagaaacagcagaggtagcggccagtgagcgcttcctccatccggggctcgggcggcgagctggctgcaagactgggcaccctggagccgtgggagccttgggagccgaatctgggttcgaatctgagcagggaagcggtgccgaccgcggaggatccaaggcagaaggcccagcgcaccgccgccgccagctcctgcccaacccggctccgctgggcttttcctctggggtggcgccggccccgcgcccctccccatccggggcgcctctgctaggacaccggggtctcggggtttctcagggctgctgcgtttcacctcctttaacgcggaggcgcggagttgcacgtgtgggtctcagtggagccgccacaggtcttattacacaacaaagggcagggagggcaaggccaggagcctcgcggggcggcgcagagaccccgcatcgcgggcgcgacccctgcctcgggctcaggctgactctgcggcagctccaagtcccgttgccgggcaggacttcggtgctccggaagggtgcggggcagcaacctggccagggcagcgcctcggtttccttccaattcattctcgattcccagatactcagatgagaaatccgcactcaggccgtcaggctcctgggcccgggccaccctgcctgcgctcagcccggcccctccgccgcagcgcctcggagtttgggggctacaggggaagaggaagcgtctggtcggctcccgtccgagctgctccacctgcgccgctggcctttccgcccgctacgcacctgggccccgagaggccagaggggagtgtccccagtggcccgggtccccgaaggtggcccagctcgggcgctcgcgcagccgggtactcaccttgggggcgcggcggcaggagagcgcgggcggagaggtgcgggcgccctgggacgaccagccgaggacgcctggtgtttggacggctgcccccgcgctgtccccccgtgcgaggcgtgttggcctccgtggcctcgcgagggaccgagggcggcgggtccgagtgccgcccgccggcctgggcagtggcggagcgcggagctggcggcgcctccgctgttgtttttgagccgcgaagcccgaagcagcagccgttgggtgggggcgaaggggaggaggcgggggccgggaggcgcgaggggggcggtggggcccggagggggcttcggaggagccgggttctggcgcgcgagccccgacgccccctccagcgatcgcagggcggggacgggggccgaggggagaatgcaggagaaaagcgggccggggcgacggggaacgcaaagcttttaaattagtaatcaaacgcagtaaaacggagcaaaaccagcccggactcgcgtccgaactatcggcgcgtgtgccccgagatccaaggaagtcgtgtggaaaaccgcagccgccgccccatcttcggccccggctggggcgggagcagcagcgcgacgcgccgcgacgcaggggcagggtggggggatagtcacctcgcctccggggccacctgggtccttttaaagtgtccggggtgcgggccccgggcgggagcgccgaacacggaatgttttcttaaagggccagttccgagctgcgctgaaaagggggggggggagaggggggagagataagtgagacgaagaccgagaaggccgaggagagccccccaatcccgcgccgaggcggcggcggcggcggcggcggcgcgacgatgaggatgatgaatacattgcaaagtttttttggccccggcgaggggtgtcagattgagtgctctgtgcgcatgtgcgaaggtgtccaaactgacaatgctggggagatgaagatagtgtgtggctgcttctggactcaaggaggaggagagagattccgcgagccgacaccatgcgatccaaggcgagggcgaggaagctagccaaaagtaagtctcccgcgctcggccgcgccgcgccgccggggcccgggccgccgggccggggcgcccgggccaggggtgcgcgtcggggcgcggccggcgcgcctgcggctccgggcccccggctcggccgcgcggcccggggggctgctccgcctcccgcgctccggggcgaccgggctcggcgcggaggctcggggcgcccgggccgcgcgctccccgaaggcgccggccccctccccgcggaaccccctccccccccaccagtgtcaggcgctcgggcccgggaacccgaggcgcgcggtgggggccggggaggggggcggagggggagggccgggggtggaggggagcgaaaagtgaaagttagcgaaaagttgacgaaaggggagagcaactttttcctccccgctcgctctctccctctctctctccgagtggctctcagtcctggtcaaatcatattccgggcttttgaaatagtgggcgctagagcaccgcctttggcgtccgccagccgggcgcagaggagggagaccccgagccggggaggggcggaggagggggcgcgggccggcgcccacccggctccgcgggcgcaggggcaggggtggcgacggcgggacagccgcagccacttggggagcaaaatggagacttttgcccgggctgggaactgtggtcgtgaagtttatccccggctgtgccccgcgtggtggagggcgctcctctgggacgccggcagcctgcgcgggccgccggggcgacagtgcctccgacaggagggacccacccggccacctcccgacccgccgctgccgccctcgggcccgcgtggacccctctgtacccgcttgcctggcacagccgggcccggcggggacagagctgccccggccaggctgcccggtcctcggtcttactttctctttcgctccgtctcggccgagccccgggctccgcgcgccccgcctgccgccagccggtccttgccgcgggccgggtgctccagccacccgcgccctcgcccgccacagcgcctccgagcagcccgcgcgcaccccgcagccccggtcgccggcgccatccgcggtgaggcgggcgggcaggtgaggacaggcggagccgcggcccggccagcgcggctcccgcagccccgccgaagccccggctgcagcgaagcggccagcgccgcgcggagcggaatcggcgccgcctgctcccgccgccccctcctctgcaggccgcccgcccgcccacccgcagaagaggcgcccgggccgctcccctcgaggtccccgcccgacccacgccgttccgtccgcgcctccggggccgggtccctccaggccggcctcgcagctcccggggccgagcccccaaacgtgagccgggtgcgcgcgggactcgcggcggttttccgaagctccgctccctcggctcggggcccgcccggctcgtcccaacagctgccccgcggccgtaggccccgcgctctgtgcaccagggaaaagcagctcccgagtttgtgccggacgctcctgcgccccgccatcggccgttcccggcccctgggtgaggcgcccccttcccgccgcgctccgctgccgggtgccctcggcctcggcctcccgtgacctctgacccctcttggtgcgaacaagaccgggcgtttcgccgccgacgcgaaggggctgtctgtgcgcggcgttgcgggccctccgcgcgtggggtgtgcgtgtgcgtgttcgggttcggttctgtgtgtgcaccgcgggcctgctcagagtcgggaccaccgggctgcgtgtgggccgccaggtcaggccctcgcccaggatgggccctaagctggaacgagaaaggggaaaccggccgcgctgacgccggggccttcgctgcaagcgagagg

SEQ ID NO: 82 - DNA, 4001 bp, Homo sapiens
  source 1..4001: mol_type = genomic DNA
tggaggaggaggcagggagagggcctgagagagttggggcacccgtagaagaaggccaggactgcacaaccttggcatctgttggctacctccaggccaggggcaaaggaggcctctgctgggctgcagggtgagggtggggaagggaccctctctgcaggtgggagtggttcgcaggcaccatagtaacttccccaggcctcctcccaaacctgcagaggcttcaggcaggagcaagggggacctgggtgcagagatatgggagtgaccttgagtcctttctagccagcagctccagccaggttgggcccaccgttgggaacctgcccttggcggggtttaggcaagacagcgcccgtgttcctgggctccagcacctcccacccactggggcgagcaggttcagaaccggccttggcggtgcaaagctgcagcggtgccctagttggctaggcagatcaagacctccagagccgggcctgtaaactgagctgttctctgccccaacccctgtagaaagatggtcagggtgtggggagggtgggtaaaggggtcgatagaccctcgaaaggacggcagggaatggggctgtgccattgctcatggcccttctctaggcgacactcacctggtgccagaaacagcagaggtagcggccagtgagcgcttcctccatccggggctcgggcggcgagctggctgcaagactgggcaccctggagccgtgggagccttgggagccgaatctgggttcgaatctgagcagggaagcggtgccgaccgcggaggatccaaggcagaaggcccagcgcaccgccgccgccagctcctgcccaacccggctccgctgggcttttcctctggggtggcgccggccccgcgcccctccccatccggggcgcctctgctaggacaccggggtctcggggtttctcagggctgctgcgtttcacctcctttaacgcggaggcgcggagttgcacgtgtgggtctcagtggagccgccacaggtcttattacacaacaaagggcagggagggcaaggccaggagcctcgcggggcggcgcagagaccccgcatcgcgggcgcgacccctgcctcgggctcaggctgactctgcggcagctccaagtcccgttgccgggcaggacttcggtgctccggaagggtgcggggcagcaacctggccagggcagcgcctcggtttccttccaattcattctcgattcccagatactcagatgagaaatccgcactcaggccgtcaggctcctgggcccgggccaccctgcctgcgctcagcccggcccctccgccgcagcgcctcggagtttgggggctacaggggaagaggaagcgtctggtcggctcccgtccgagctgctccacctgcgccgctggcctttccgcccgctacgcacctgggccccgagaggccagaggggagtgtccccagtggcccgggtccccgaaggtggcccagctcgggcgctcgcgcagccgggtactcaccttgggggcgcggcggcaggagagcgcgggcggagaggtgcgggcgccctgggacgaccagccgaggacgcctggtgtttggacggctgcccccgcgctgtccccccgtgcgaggcgtgttggcctccgtggcctcgcgagggaccgagggcggcgggtccgagtgccgcccgccggcctgggcagtggcggagcgcggagctggcggcgcctccgctgttgtttttgagccgcgaagcccgaagcagcagccgttgggtgggggcgaaggggaggaggcgggggccgggaggcgcgaggggggcggtggggcccggagggggcttcggaggagccgggttctggcgcgcgagccccgacgccccctccagcgatcgcagggcggggacgggggccgaggggagaatgcaggagaaaagcgggccggggcgacggggaacgcaaagcttttaaattagtaatcaaacgcagtaaaacggagcaaaaccagcccggactcgcgtccgaactatcggcgcgtgtgccccgagatccaaggaagtcgtgtggaaaaccgcagccgccgccccatcttcggccccggctggggcgggagcagcagcgcgacgcgccgcgacgcaggggcagggtggggggatagtcacctcgcctccggggccacctgggtccttttaaagtgtccggggtgcgggccccgggcgggagcgccgaacacggaatgttttcttaaagggccagttccgagctgcgctgaaaagggggggggggagaggggggagagataagtgagacgaagaccgagaaggccgaggagagccccccaatcccgcgccgaggcggcggcggcggcggcggcggcgcgacgatgaggatgatgaatacattgcaaagtttttttggccccggcgaggggtgtcagattgagtgctctgtgcgcatgtgcgaaggtgtccaaactgacaatgctggggagatgaagatagtgtgtggctgcttctggactcaaggaggaggagagagattccgcgagccgacaccatgcgatccaaggcgagggcgaggaagctagccaaaagtaagtctcccgcgctcggccgcgccgcgccgccggggcccgggccgccgggccggggcgcccgggccaggggtgcgcgtcggggcgcggccggcgcgcctgcggctccgggcccccggctcggccgcgcggcccggggggctgctccgcctcccgcgctccggggcgaccgggctcggcgcggaggctcggggcgcccgggccgcgcgctccccgaaggcgccggccccctccccgcggaaccccctccccccccaccagtgtcaggcgctcgggcccgggaacccgaggcgcgcggtgggggccggggaggggggcggagggggagggccgggggtggaggggagcgaaaagtgaaagttagcgaaaagttgacgaaaggggagagcaactttttcctccccgctcgctctctccctctctctctccgagtggctctcagtcctggtcaaatcatattccgggcttttgaaatagtgggcgctagagcaccgcctttggcgtccgccagccgggcgcagaggagggagaccccgagccggggaggggcggaggagggggcgcgggccggcgcccacccggctccgcgggcgcaggggcaggggtggcgacggcgggacagccgcagccacttggggagcaaaatggagacttttgcccgggctgggaactgtggtcgtgaagtttatccccggctgtgccccgcgtggtggagggcgctcctctgggacgccggcagcctgcgcgggccgccggggcgacagtgcctccgacaggagggacccacccggccacctcccgacccgccgctgccgccctcgggcccgcgtggacccctctgtacccgcttgcctggcacagccgggcccggcggggacagagctgccccggccaggctgcccggtcctcggtcttactttctctttcgctccgtctcggccgagccccgggctccgcgcgccccgcctgccgccagccggtccttgccgcgggccgggtgctccagccacccgcgccctcgcccgccacagcgcctccgagcagcccgcgcgcaccccgcagccccggtcgccggcgccatccgcggtgaggcgggcgggcaggtgaggacaggcggagccgcggcccggccagcgcggctcccgcagccccgccgaagccccggctgcagcgaagcggccagcgccgcgcggagcggaatcggcgccgcctgctcccgccgccccctcctctgcaggccgcccgcccgcccacccgcagaagaggcgcccgggccgctcccctcgaggtccccgcccgacccacgccgttccgtccgcgcctccggggccgggtccctccaggccggcctcgcagctcccggggccgagcccccaaacgtgagccgggtgcgcgcgggactcgcggcggttt

SEQ ID NO: 83 - DNA, 2941 bp, Homo sapiens
  source 1..2941: mol_type = genomic DNA
cactccagcctgggtgacatactgagactctgcctcaaaaaaaaaaaaaaaaaaaaaaaaagggtaggcatgaggcagactgtgaagctgagtggggaaacaaggtgaaggaaggggataaaatgtcctctgaggagacctgggctgtcagaggccagggaaggggacgagggttggggaacaggtggttagcacttcatcctcgtctccctcccaggttagaagggcccccctctctgaaggcccccactccctgggatgctacaatccaatgatggaagatggcattagctacaccaccctgcgctttcccgagatgaacataccacgaactgggtactgagggtaccaggagggtgaccctgcaccctgggagggaggcgggaggaaaagctctgtcccgcctgccctctttgtgccaggcccatgccaggcaccctgatacatgctctgcctcattcccactcggcaacaagcctctgggtaagggccagtctccagaagtggacctgctccttcaagtcagcagcacacatgcccagtccgttcttatcacaaagatcaagacctcatgatacacacccacgcctactccctccacgcacatttgcacgagctcctactgtgagctttgggcactggggaggccacaggttttacataaggggctgaggttggggtgctgttgggggctctgggtgttgagagggaggagagttcgtgggagatgcttcatgcgtggtcgtctatctgccctgtctctcagagatgcagagtcctcagagatgcagagacctcccccggactgcgatgacacggtcacttattcagcattgcacaagcgccaagtggtaaggagggtctccccaggtctccccagaggggctgtggaaggctggggacagggcctggcctcagtggtgggtcccacataggaaggagttgggtaggcatccgtggtggcagaggttgggtgttggagacggttgctccggcagagctggccagagccaggcaggtacctccaggtcctggatgccggccacagccagtttcctgacacgaggacacccgcctgggcttttggacccccgggtggaatgaaggagagaatgcggagaaaagcggcggtggaggctggcgacagtggggccaggctaaccaccatgcggttttctcagggcgactatgagaacgtcattccagattttccagaagatgaggggattcattactcagagctgatccagtttggggtcggggagcggcctcaggcacaagaaaatgtggactatgtgatcctcaaacattgacactggatgggctgcagcagaggcactgggggcagcgggggccagggaagtccccgagtttccccagacaccgccacatggcttcctcctgcgcgcatgtgcgcacacacacacacacacgcacacacacacacacacactcactgcggagaaccttgtgcctggctcagagccagtctttttggtgagggtaaccccaaacctccaaaactcctgcccctgttctcttccactctccttgctacccagaaatccatctaaatacctgccctgacatgcacacctccccctgcccccaccacggccactggccatctccacccccagctgcttgtgtccctcctgggatctgctcgtcatcatttttccttcccttctccatctctctggccctctacccctgatctgacatccccactcacgaatattatgcccagtttctgcctctgagggaaagcccagaaaaggacagaaacgaagtagaaaggggcccagtcctggcctggcttctcctttggaagtgaggcattgcacggggagacgtacgtatcagcggccccttgactctggggactccgggtttgagatggacacactggtgtggattaacctgccagggagacagagctcacaataaaaatggctcagatgccacttcaaagaaccagtgaactcttttcagtctgtggagtcagagtggggatgaggagaggggaaagtcttcatggggcctggggggaaggaaatggagtcacagtggtttactgatcaaaccagcagattagagagtctggtgtcttgttaggaaaagcatactggctttgcagacagacaaacatgagaacttgaatcttgattcttcggcttaactagcccggtgatcttggcagggctcgccagcttgcctgagtctcggcttctccatctgtaaagtggagagggaaaggccactgcctaccacgcaggtggcccacgataggaagtgaggcatcgtcttctatatgctggagccacattgagacccagcacagaagccgaaagtcccccagaaaaccaacctccatcctttatttgcatttttcgaagaatgtgaaaaggggaagccgattcaaaacagctcttcccaaaagtgacgctacccaattcttgcacaacccatagctgccagagaaagtcttgaagaccagccaccctgagaaacctcacagcttttctgtcattcgaggtcctctgcacccgttctctaattctgttcactgattcagactttccagttaggtgccccagctcccctctccgcttctcgggtttgatctttctccacccattcacctgcttctgcaaaggggctaaagcccattctccttagagggcaggggtgtaattatctgatcatcagggagattggctcctggcacgtggcaggtgtccccaaactgccaccttcctctcccaccttcctttccctcccatctctgctcagtacccggacggtaactctaggaattatggttcagggaggccttgatcgccccatcaatccactttgtccctggtttgattcttgtgagcaccattaacttttctggaggaaggtgaggctttaagaactgtttaaagcccaggcg

SEQ ID NO: 84 - DNA, 4001 bp, Homo sapiens
  source 1..4001: mol_type = genomic DNA
aggaggttaatgttggagaaggccttttagtgcaaagagcccaccctggttgtacattctagggaaacctctctgtgtctatctcatcacatcagcctgtgatgtaatcaatctctctccgtctctgtctgtgcttttccctcttactttctctctctctctctctctctctctctcacacacacacacacatacatatacctatagtttcctttccctcacacaaacactaatatcccctgaccctaatcagacagttgccaaaaccccatttgtctgagttccctttatttcaattttttgccctttttcaactgtttcaggaagcaaaccagttctgtttttcaacttgaacctagtcaatgttcaagctgagcatctacttctcaggttagaagtcacatcatcagctcagacctgtttgataccgtgaaaagcagacacacaaatggaccccctccccagacagtacttcagactctggcgcaaccagcctgaagtactcaggtttctttctttctttctttctttttaagatggtgtctcgctgtgtggcccaggatggagcgcagtggcctgatctcggctcaccgcaacctccacctcccggtttcaagcaattctcctgcctcagccttccaagtagctgggattacaggtacctgccaccatgcccagctaatttttgtatttttagtagagacggggtttcaccacgttggccatggctggtctggagctcccgacctcaagtgatccacccgccttggcctcccaaagtgctgagattacaagcatgagccactgtgcccagtcagtgcttagatttcttaagacccaaccttaagaaagacaattagaagacagagtgggcaactgggagaggcctggtaatatggcagagggaagagggggtgaaggtgtgggtggcaggagggtggctggttggggaagtggccactggatcttaggccacaggcatacttttccatctccagcttcctcatttacagaaaacgagactgcagtggtgtgatggaaatgtgtgggacggggacaggtgactggctttcattactaaccagaggacagcttagacaagtcactctggctgggcctcagtttccttatctctaacgggactggattgcccagtgctcctcaaactgtggcacctggggcacattcatagcttgtgtgtgaatctgccagccttgaaatatctgtcttccttggatggcaagtttattaaaaattttggaaggactcactctttttataagaaagcatttatgtcattgttcagaatgcaaaatgtatatgcattttagttacaataactaatatttgataacatttaacgagtaattactgtgtgccaagtgtgcaaaactctaggtgagcctcacaataacctaatggggtattattattcccattttatagattaaaaatctggggtagagggagtagataatccacctaaagacctgttattaagtagtgataaaagatctccactccctctctcccccacaaaaaatatggactcattacaagttgttttaggatctacctccagacccatggagtttctttagtaaagcctgaacgacacaggccaaaataatctccaaaggccagctctgacccttttaaatcaattttagctaaatccgttcacaaaaggcttcgcacatccagtgtccctgaaaaataaaggaggttgggcaggccctgcgggggctcgaggaattcgctaagtgagttttctggcttctggatacactttcaaagggccagagggcacgaggcttccgccttggccgccacctccccggccagctgcggtgttcgcggccagtgttgccgggcacttcctggttcccgcgcgccccgggtgcagctccctgcacccagtgctggcgctcctcagaagggagggggccagaggcgagatgtcgcaaccgcctccctccctctttccccgccttggcactcagtcgcctcccagatgagcactctctcagaccgctgcgggccgccaggcgccgggaatgtcccctgaatgcgcgcgggcagcgggcgacgcgcccttgcgcagcctggagcaagccaaccgcacccgctttcccttcttctccgacgtcaagggcgaccaccggctggtgctggccgcggtggagacaaccgtgctggtgctcatctttgcagtgtcgctgctgggcaacgtgtgcgccctggtgctggtggcgcgccgacgacgccgcggcgcgactgcctgcctggtactcaacctcttctgcgcggacctgctcttcatcagcgctatccctctggtgctggccgtgcgctggactgaggcctggctgctgggccccgttgcctgccacctgctcttctacgtgatgaccctgagcggcagcgtcaccatcctcacgctggccgcggtcagcctggagcgcatggtgtgcatcgtgcacctgcagcgcggcgtgcggggtcctgggcggcgggcgcgggcagtgctgctggcgctcatctggggctattcggcggtcgccgctctgcctctctgcgtcttcttccgagtcgtcccgcaacggctccccggcgccgaccaggtgagcgcccctctgtgtgtgccgggcaggtgtcctgcgcaggctgggaagcggggccccgacggaagctgggatgaggatgatcaagaacaacaatagccatttattgcacttaatcgttgtgccaaatcttgtgcccatggctgtgaagtttaatctcttaaatctcactacaacgctgtgcacacgccctcctaaatgatgtaagtggagtcccccaaattcttgcaaaatgcaatgactgttgcgaggttaattaacgagtagtttaggagcgagacggaactttgggggtgcagggtggccaaacactttgtattgaatcatgattcctcgccaggtgctacaatactgttattatcacacccatttcacagatgagaaccagaggcacaccgaagtgtataataacttgcccagagtattttatccgtaattcgaggaggagatgggctccttccagaagtttacccgtaattcaaggaggagttgggctcctgtccagggttgggttatggtcctgctttgaaagcgcgcggacaggcatgtgagacccggggaccccagatgcaatgctgtctttaggggacttgtgacagaattcccttccggggtcttcagttttttcagctgcaaaacggaaggattacactagaccttcgaggtgtcctgggcgcctgaaatgtgcagattacagaggctggaccgacgagctctgttttacatagcgggtcaatagttgtcattattagctcataggggtgggagtgaccatgatcggaataaggttctgcggatagtgcaggacattggctctgttcccctcaagtcccgcggtccggaatcccgagctcgggtctttttctcttgccgctttgcccggtggttgcagggttcgtgcgccgggatccacacggaagagggaatctagacgcggagtcacggtggaggacagtggccgccgtctggctgtcttcccagtgtgtttggttctccatgcctcgcggagagagatagagaaaggacttaggtctgtgcgctgggggctcagcctccaggagcttctgcccgtggctgggttcttggcctccccttccctgccggggtcactggttttccggcctggaaccccctacccttaagctccctctccacctaggagtgaatatggggcgccctgagtaatcgtggcacaaacaggaatcacggtcccaaagagggaccttcgaagtggtcagagaggggagtagggagggagaaatgacatgtcaccaggggaaggcttggttatggagatgcacacagcccaccagggacaccccctgactgtgtgaccctccagagcctgagacagagaggtggagcttctctgggggt

SEQ ID NO: 85 - DNA, 4001 bp, Homo sapiens
  source 1..4001: mol_type = genomic DNA
ggtaggaagtgggcaagagctgctatggcctctttgcctccactaggggatggatgggcttcctgaaacaaacccaattcagagaaaagagtcagaggtgaggacaggtgcccaaataagttgttgttgtttgcatccctggatccagatgtgcctaaagctaaagtcactgcctggattttttgcttatccaggccagtgcatttctttttttctttctttttcttttcctcctcctcccttttatcctccttctctcctttcttctcctccatttctgtttgttttatttgagttggattcctgttagttgtaacccaacgtctcccgatcattatattcttctgcctcagtcttctcacctgtaaaatggggatgataataataacaatgttcttcttctgcacaggattgttgtgagacttagctgagtcaccacaagtaaggtgcttgttacatagtgagaggtcagtaaatgttacctattaaggagttggaaaaagctgaattcagagaattttatctccctgctgggggcggtggctcacacctgtaatctcagcaatttgggaggctgaggcaggtggatcatctgaggtcaggagttcaagaccagcctggtcaacgtggtgaaaccccgtctctactaaaaatacaaaaaattagccaggcatggtggcacgcgcctataatcccagctactcaggaggctgaggcaggagaatcacttgaacctgagaggcggaggttgaagtgagccaagattgcaccattgcattccagcctgggccacagagcgagactgtctcaaaaaaaaaaaaagaaaaagaaaaaaagaaaaaagaaagttttatttcccctcccccgcaaaaaagaaagagatttctccatgcatcaaaatatgaggaatagattcctataatgcaaattatgagaaaagagaaagcactgtgtcagtcagttcaccagggagtcattattccagaatttatggcctaggagtcactgacctcacctcacgcctctgtctttctctctttctctcactctcttgcttgtgtgtgtgtgtgtgtgtgtgtgtgtatgtgtgtgtttcacaggaattgcgagagaggaggagaaaggggagatgaagtggttggctggacagaaatggctggttttgcaatgcagtttggggagaggaaaagtaaactgggagaaagttatccaggccttagagcgtccttcctgcatgggttgtagaaagagaggacatggggcttgccattcggaagctgctcagctaagtctggttatttgtctggggagtcaggagtctaatgaagccaggccagaggcctctaaggctgaacaaaacattagcacaggaggagcaacagttacacacacacacacacacacacacacacacacacccattctggctggattaaacagaaaaaggtataatgtaattcacaggctctccgatgggagggccagagaattgaccgtgaagacttcccagcctggaacgatgtcacatgatgaaggctgctccagtggaacactcagtgtcaccacactacagcacagacaccatgcctggcttgcaccactggttctggatgtgagatcctagaagcctcttgattgtgccctaaactcttgcccataaatggattctgtgaaaccctagcttgtcctctcctgcaaaggcaagtctctcacggtatgtctggtggtctaagcttagggtacatgccttggtgcaagggagatgggaaagccagtgttgacctttcccttggggaggcaggactcatactgtgggaaatttgcaagtcacctgatagagttcaaaaggacaaccatcaagtctgatatatatccccacaatagcattgctatattgggtgtgtgtgtgtgtgtgtatgtatgaaatagacaaatgatctatatctcaaggtccactttccaggatggtttcatccttaaaaatccaaagaaccatcattcaagctgccaccaggagcagggagcccagttccctcatttgggtgtcactcaatacctctgagtccagacaactgttccaagctccaggtaccgtctggtcaccccttctccttgtggctctgcttgttcaggtcagcattgcccccatatgtgggcccaggtccctttctccccttacccccgaaatgaaaagctgacacactcttcagagatgtaccaggctcatggatggatggacggatggatgggcgtataggtggatgagggggtgggtggatggacaccagaggtttgaggacaatggtcactgtagccaatgacaccagggggcagtgcggagcagcttttcaaggcctaactccctacactcttccatcagacctggctggcagggcttggagagctcattttgttcaacattttcatttgtgaggaagttgttgaggcccaagacaggaatggctgtgtcagtagcgcacagcagctgagctggctcttgatgcggctccagcaccctctgcattacagtatttacataagcccaccttcccaggctcggagggcccccacgctgccaagcctccgtcagctatcagtgattctggagcccttcctaggagtctcatcggttgccggctgctgaactcaatcagcatagaggaaacagacagtcagagttcagggcgttggccaggctgagtcattgtcttcgcccctccgaagggctgcatgttagggccctcactggacaccccttccctagccatgtcttttctctccttttggaatccagatcacctggaggtcatatttcccaggaaaggaaggaccccacgctgagaacacatcttacaataaatgatccctctcttctcaaaatttacgtctgtgttacatcccctttagccctgctttttaatttccactgcctgtttctttgggggcagtgggggtggagtgggggagtaccagggtggtgatggcccctcccacccccacccctatccttatccatctttacagcaggctcctgaggaccacgacttagatagcagagacatcaagaggctcactaaacctcaaatttccattttcgttcattcatctatttaattattcaactattgaaaatattttaagcccctcctatgtgtcagatgctaaaaaaagtcacaaacttacacttcttaagcccttactacactaaaggtgtgacatttagtatattatcttacacaagccaatgaaggaagtattttatttatttatttatttttatttttttgagacggagtcttgctctgtcacccaggctgaagtgcagtagcacaatctcagctcactgcaacctccacctcctgggttcaagcgattctcctgcctcagcctcctgagtagctgggattacaggtacctgccacccatatcattcaattcacaagcattttaatgactgtgtagtattctactgaagacatataactgatttacctcccccttactgttgaccttatatctattgtagaagtggtttagtgtttcagtgtttaatattttctttctttctttgtttcttttttttttttttgagacagagtctcgctctgtcgcccaggctggagtgcagtggtgtcatctcggctcactgcaacctccgcctcctgggttcaagctattctccctgcctcatactcccgagtagctgggattacaggtgcccaccaccacgcccagctaatttttgccatggtgaccaggctggtctcgaactcctgaccttgagtgatccacctgcctcagcctcccaaagtgctgggattacaggcgtgagccaccgtgcccagccttgaaggatgcattgttattttcgttttacaaatgaagaaacagagatctagagaagtgatgtcacttgcccaagagtgcacagccagtgagctacaggacaggaatcaaacccaggaggccacacccaagtatcagacatttcctgcaagtgt

SEQ ID NO: 86 - DNA, 4000 bp, Homo sapiens
  source 1..4000: mol_type = genomic DNA
ccatgctcaagaatagggtggcaaggcaggaaaagactcacacatcccagcccccggcccctcagggcagcctctgtgtttctaactaaaagcctcctaccaggggcaagaccaagttcgggcgcttccccgcatggagaaacgctggccccacggcgcccggccccgacgcgctggccctttaaaggcgcgggcttcctgcgcccggggcccctcccacgccaccgccaccgccaccgccaccgcccgcgcccgcccccgccgagggtcacacttgaactttagctcgccccgcgcggccaggcgccggcgctccgcaggcggtggccgccccttcgcacgtgtcggaggcaccacctcgggctcgggacgccgggcccccggcactcgaggccactccggagaaaaccaaaacaaagcaaaatgcgcaccgcctccagaaactgcccggcaggccctgcaccgtccccggccggtcgcgccctgcctgtcccctccacctgcagcccggtcccggggcagcgaggctccggggcccctcgccgcgccctcccccgccgcgcccggcggaaacagctccgcggcgcggccggagagaacccgccctccccccgcggaggtccgggagggaaggggcagccgaagcagtcggcgcgggccgggggttgccgctcccagcgaacccctttctcctttcactggcaaacttttcggcctcgctctgacgtccacttcttggcgcactttctttacttagttccccaacgagccccttaccgcgtcccacgcgaactcctgactggcgcgcacgcacacctactgccgtccccgaccggacccgggcgaggccaccgcgaccaccgcttctcgcccgccctcctgggaacgcgctgccctcctgctccgcaccttcaggccgagcaaacctgcacagctgcgccctcgcctgacccaccgcgcccccaaggtccggccgcgcgccgagtccactcaccttccagcccgccgagctgttgctgtcacccttatccttgaagtagggcacgctcttgaccatccactcgtagatctgcgacagcgtgagccgcttctccgccgagctctcgatggccttggtgatgaggtcggcgtaggacaggttgccccacgcgttgcggcgggacgagctgctcttgcgcggctgccccgcgagcggcccagcggcggcggggggcaccggcgggtgctgcgacagcggcccgggcggcgggggctgcggtggcgctgggtgcaggcagcccgcctccgggccctggaagtccccgcacagccccccggtggcggccgcggcggccgccgccgccaccgccgccgccacggagccgggcgcctgcgggaagtcctcgctctcctccagcaagctcaggttgctcatgaagtcggcgctgacagcggcagccgaggccgagggcaggcccgccgcggcgtcggggttggcagccgcgctgcccgacggcgccgggctggaggtggccgagttggactggctaaactccggcctgggcagcggccaggtgcacgagcgcggccggggcagcggctcgaagtccgggtcgatctccaccacctgaggcgcctcggccatggtgacccccgcccctcccccagccgcaggagagccaagagggggagaacgcagcactgggggcggacggggagggggcgcgaagggacggtccgagatttgggggaacgaagccggtgcggcgagcggacggaaactgggaggaaggcgcggcggagtggaagcgcgagcccagaacttaacttcgcggggccatccacatcgaggctcctcggggtccgccgcacggactggacggccggccagagccgccgggccggggcagagcctgcgccgcgctccagctgacagggccgcggacggaaggacggacggacgccgcgggccgcttgctctccccagcggcgcgcccgctgcgctgctgcctgttgaatgtggcggctgcggcagcggctgctgcgactaccaggccgcccgacttacgggatctgccgccgccccccgcccgcggcggcgcgcgcgccggcccgcccctgaccgacagcccgcgcggccaatgggcatgcggcaccgccgcccgggcagccagtgggcgccgggctgggtggggcccggttttccacggggaggcggcggtgggctggtggggggtagtggggtgtttttctctttcacacactcacctcctttttttttttttggatctctattattttctggtaattctcgagtgtttctgtgattctctcgccttctcagtgttttgattgctaggaagcaaaccagcgtggaggcgccggcgacactttgtttactacggagcagcagagccgagtactcgggaagcccgggtgggaggaggcgctcgctgctccctgacctccgctgcgggccgagcccggcgggctggcagggcagggggccgagggccgggggcgcggggtgggcgggcggaggcggccgcgaggaattctactcaatcgctccctcctggctccacccacgatgtctttgctgaacgacgtggggaatcggtgggttttgttttggtttaatgttttctttcgctgcgatctgtcaagtcctccggccccctcgcgagcggcacacgccccccacccccggcgccgcggctcctgcagtcgagtccgcgccgagggacccttctccgtgcccaccggtccgcacccccgggctcagcctgtgccattcggtctagccagaggcgcgtcacccagccggcagcccccagccggattcactgtattcttgaccttttttaaaatgctagaaatgaggaacaggggctccggcgcggggaggagattatctggcctccctgaacacacgaatccaaaggcacccgcgaccgcatgccctccgtgggaacctcttcccctgggcgggaatctcccgctccgtccactaagtccagggccggcagcgaagggctcaggagcgcagttagagagaaccaggtcattttattacttatccctaattaaatttagctgtttgttacttcttttttatttgcaacggaagtgatcgggtgttaagtaaaaacgcaaggggggcgggagataggaccaaagccttggggcgcagcctgccccacccccaaattccttgaggctacttgctgtgtgtgtcagtcaaccccccacccacaccccacctttcctcatctgggctgcaggtccaggacatcctgtcaaggatcagtggggccaaaggtgggaagataatggcccttgtgtctcaagcagaagaaacagttttccagttttgttttgttatgcttttgcttttttaggaagaatatggaaggcatttccttttagaaagatgggaccacttaggatggaagatggacagtagggttttaaatatcttgtccaaaaggacagcttccaactcttgttttttaatccctttctttcttacaagtaattctgcaatggaaaaagtggcatctagagaaaattagtcagttggacaagaggttgttgaaaaaataaaagagaaaagaggggaaaaaaggaaaaattaggcctcttacaaaagttaaaaagattgagagagattatctttcttcttttatattctcactatcgtctctctagtccgggctcctgtttcttcataatctatttatcacaacagcttcttgctgatactaggcatgggtggggtggggagcgtgagccggatggattggggacgggcggggactggttcccaccctggtcttgtggtctcttcacgtttactatcaaatttggacagtctgggcctcctctgacagccgctaggggacctcagtaactcagatgatgagctcaaaggcaggcccccaaagtacagctgttgcttccccccacctttccaattacactctgcagaagcagcccccgatccagacagccgcaccct

SEQ ID NO: 87 - DNA, 4000 bp, Homo sapiens
  source 1..4000: mol_type = genomic DNA
ccgtccccggccggtcgcgccctgcctgtcccctccacctgcagcccggtcccggggcagcgaggctccggggcccctcgccgcgccctcccccgccgcgcccggcggaaacagctccgcggcgcggccggagagaacccgccctccccccgcggaggtccgggagggaaggggcagccgaagcagtcggcgcgggccgggggttgccgctcccagcgaacccctttctcctttcactggcaaacttttcggcctcgctctgacgtccacttcttggcgcactttctttacttagttccccaacgagccccttaccgcgtcccacgcgaactcctgactggcgcgcacgcacacctactgccgtccccgaccggacccgggcgaggccaccgcgaccaccgcttctcgcccgccctcctgggaacgcgctgccctcctgctccgcaccttcaggccgagcaaacctgcacagctgcgccctcgcctgacccaccgcgcccccaaggtccggccgcgcgccgagtccactcaccttccagcccgccgagctgttgctgtcacccttatccttgaagtagggcacgctcttgaccatccactcgtagatctgcgacagcgtgagccgcttctccgccgagctctcgatggccttggtgatgaggtcggcgtaggacaggttgccccacgcgttgcggcgggacgagctgctcttgcgcggctgccccgcgagcggcccagcggcggcggggggcaccggcgggtgctgcgacagcggcccgggcggcgggggctgcggtggcgctgggtgcaggcagcccgcctccgggccctggaagtccccgcacagccccccggtggcggccgcggcggccgccgccgccaccgccgccgccacggagccgggcgcctgcgggaagtcctcgctctcctccagcaagctcaggttgctcatgaagtcggcgctgacagcggcagccgaggccgagggcaggcccgccgcggcgtcggggttggcagccgcgctgcccgacggcgccgggctggaggtggccgagttggactggctaaactccggcctgggcagcggccaggtgcacgagcgcggccggggcagcggctcgaagtccgggtcgatctccaccacctgaggcgcctcggccatggtgacccccgcccctcccccagccgcaggagagccaagagggggagaacgcagcactgggggcggacggggagggggcgcgaagggacggtccgagatttgggggaacgaagccggtgcggcgagcggacggaaactgggaggaaggcgcggcggagtggaagcgcgagcccagaacttaacttcgcggggccatccacatcgaggctcctcggggtccgccgcacggactggacggccggccagagccgccgggccggggcagagcctgcgccgcgctccagctgacagggccgcggacggaaggacggacggacgccgcgggccgcttgctctccccagcggcgcgcccgctgcgctgctgcctgttgaatgtggcggctgcggcagcggctgctgcgactaccaggccgcccgacttacgggatctgccgccgccccccgcccgcggcggcgcgcgcgccggcccgcccctgaccgacagcccgcgcggccaatgggcatgcggcaccgccgcccgggcagccagtgggcgccgggctgggtggggcccggttttccacggggaggcggcggtgggctggtggggggtagtggggtgtttttctctttcacacactcacctcctttttttttttttggatctctattattttctggtaattctcgagtgtttctgtgattctctcgccttctcagtgttttgattgctaggaagcaaaccagcgtggaggcgccggcgacactttgtttactacggagcagcagagccgagtactcgggaagcccgggtgggaggaggcgctcgctgctccctgacctccgctgcgggccgagcccggcgggctggcagggcagggggccgagggccgggggcgcggggtgggcgggcggaggcggccgcgaggaattctactcaatcgctccctcctggctccacccacgatgtctttgctgaacgacgtggggaatcggtgggttttgttttggtttaatgttttctttcgctgcgatctgtcaagtcctccggccccctcgcgagcggcacacgccccccacccccggcgccgcggctcctgcagtcgagtccgcgccgagggacccttctccgtgcccaccggtccgcacccccgggctcagcctgtgccattcggtctagccagaggcgcgtcacccagccggcagcccccagccggattcactgtattcttgaccttttttaaaatgctagaaatgaggaacaggggctccggcgcggggaggagattatctggcctccctgaacacacgaatccaaaggcacccgcgaccgcatgccctccgtgggaacctcttcccctgggcgggaatctcccgctccgtccactaagtccagggccggcagcgaagggctcaggagcgcagttagagagaaccaggtcattttattacttatccctaattaaatttagctgtttgttacttcttttttatttgcaacggaagtgatcgggtgttaagtaaaaacgcaaggggggcgggagataggaccaaagccttggggcgcagcctgccccacccccaaattccttgaggctacttgctgtgtgtgtcagtcaaccccccacccacaccccacctttcctcatctgggctgcaggtccaggacatcctgtcaaggatcagtggggccaaaggtgggaagataatggcccttgtgtctcaagcagaagaaacagttttccagttttgttttgttatgcttttgcttttttaggaagaatatggaaggcatttccttttagaaagatgggaccacttaggatggaagatggacagtagggttttaaatatcttgtccaaaaggacagcttccaactcttgttttttaatccctttctttcttacaagtaattctgcaatggaaaaagtggcatctagagaaaattagtcagttggacaagaggttgttgaaaaaataaaagagaaaagaggggaaaaaaggaaaaattaggcctcttacaaaagttaaaaagattgagagagattatctttcttcttttatattctcactatcgtctctctagtccgggctcctgtttcttcataatctatttatcacaacagcttcttgctgatactaggcatgggtggggtggggagcgtgagccggatggattggggacgggcggggactggttcccaccctggtcttgtggtctcttcacgtttactatcaaatttggacagtctgggcctcctctgacagccgctaggggacctcagtaactcagatgatgagctcaaaggcaggcccccaaagtacagctgttgcttccccccacctttccaattacactctgcagaagcagcccccgatccagacagccgcaccctaaaaataggcggtgccctactggatcattcttggcaaaggaagccagcttcaaatttggccttgccttcatttctattccggtttttcagggcgtcagagaatatttatttggttaaaggagtcttcctacagactttccccaaatccttgaaatctttgaggctctcctgcccaattgtttcttcaaatgtcatagtttacttttatttggtctttctttaaaaaaattgacgaggcaggcacagagaacctataacaccaatgtccctgacacataggtccaaaaaattaaaaatatatattgaagggtttttacagctaaccaccacactgttgtatctaaatatcaatttactgaaccccaggatgttggtgaatatttcacttgtttccagtctttagctatcatagactattctgtaagaaataaccttgcagggaggccgaggcgggcggatcat

SEQ ID NO: 88 - DNA, 4001 bp, Homo sapiens
  source 1..4001: mol_type = genomic DNA
agttcaaccacttactagctgtgtgactttggcaaacttatttcccttctctgagctccaggctagtgagtaaccagcagggctataaggtccgtgacgcttaaagtagtttggctaagaacctgctgattttctcctccacttttctgtctccacgtagtattcccttatccactcaacaaataaatacccatgtctggcatagtatgctgaatagatagttccttccctagaggaagtgacaaaagtgatctctttcagatggaaaaactgaggcccagaagttaagtggcctgcaagagccctgagatatttggacttccttgccactagctagaagagcttctcactgaacttttgtttagttatatcccacccttacggctcacctaagatcctgggagccttccccgggaacttccccggggtctcacctcaagcgagggcccaggaagggtgaaccaagtcgtctggatttatggattcaacaggtgtggggcggaaggccttttcctgccgagggtgcggcccagcgccaccccgctcagtctcagcgggacacctccacagccagtaggaagggcagttcaaaattcagtgctacccccgcgtcccagctaagaaaacaagggccatccaggtgccctcccagctgacgagatggaaaaatcaaagtataatagataaagagggaccgtttcaaaagaaaaaggggacttgaaaatattgtagactagggggaggaaaggaggtggaaagaaacaatgccctacacacaggaggggctccacaaacaatttgttgaaaagaaatcgctctgcggacgctacaaagatgccctgcaggacaagcccacttacacagaaaaaaagaaatcctatctagggagttgttggtccccagagtccccatccctcattccccaaccccgtggcgtgcgcgcttcccgctagacttcagcctggggctctttcctggcccccagatcccccgcccctccggttgcgctccccgcctggccccgagggtaaacctgcctccgcggaacacatctccgcgagactgggccaaagctttggggcctgcactcctcgcaaaggagagcctccgagaccggaggttcatctcaacccccgcgttcgagcccgggcctggagaaaagtgccctcccctcggcctcggcctcgtcaccgcgggggactggcgggaggagtaggggaggccggtgggacacctcgcaccaggccgcccacctacaggggcgtccgggctaggggagcagacggagagcgggggcggctcctgactcctccgcgcgccggggccggggccgggccgggggctcggcccgctcagccaatgggcgccgcgctcgggccccctcccccgcaccgggaggggccgaggctgggctgctgctgccgaggtcgtgtgcgacgtgccgctcccgggccctgtgtccccagccccagcagggccgcgtggacggcgcggcgcccccggaaggaggccatgtgctcagtgccggcgccaactacgcacgcggcgccctccggcccgctagatccgaagcccatccccactgcggccagcggcccggcggggcgctgcgctcgggacccgcaccgagccaggctcggagaggcgcgcggcccgccccgggcgcacagcgcagcggggcggcgggggaggccctggccggcgtaaggcgggcaggagtctgcgcctttgttcctggcgggagggcccgcgggcgcgcgactcaccttgctgctgggctccatggcagcgctgcgctggtggctctggctgcgccgggtacgcgggtggcgacgggcgtgcgagcggcgctctcccgctcaggctcgtgctccggtccggggactcccactgcgactctgactccgacccccgtcgtttggtctcctgctccctggcgtgctcactcggggacccgcgactagcgaccggcacgctcgctgttgctacctcttgcctcttgccagagagcgcgcggaccgtagcgtttataggaccccggccattggctggcgacgccggtgtgtcaggggtgtgtgggcaggacctcggggcggggcctggggcccagcctgtcctgggcggcgctctgcggggagggggtgaggggagtggccggggccggggccccccctaaccactgcctcgctcaggctgccgatcggctcgttctctctgcgttgggaccgcggagggcattcgctgtgtgactcagggcaagtgtgcgcacctctctgagcctccgcggccttctctagcaaaatggtggagccggtacccggctgtaaggcaagctgggatgcgcggagtagcgggtggaaagctggactggagttctcactcggccgctgggtgacttcggtgcactagggatagtaacagtaccacctcgtagggttgtagaaagaccaagtgagttaatatttgtaaagtgtttagaacagcgtctactgcatggaaagtagtatttatttgttagagagactccaggaggtcttgcccagggattgacttctcccttccctgtctccaccccccacattcccgtttgtgagccaagccttgcttttgcaccggccgggctctcttgcctggaaatgcatacccatctcaaacctgggctttaaaatcacctcctctgggacgccttcctctacttctcacagttcaggtgagtctatggtgacctgtaagtgtggaaaatatgaaaggcaggctaactgactgacatgataggacattaaggacatgatagacctcgggtttgaaccagctcaactgtatactggcccggccgcgatcctgggcgagttcctggccagtgaggctgttttcgtttttttctttaatttttttttttctcagacagagtctcgctctgtcgcccaggctggagtgcagcggcacgatctcggctcactgtaacctccgcctcccggattcaagcagttctctgcctcagcctcccgagtagctgggattacaggcgcccgctatcacccctggctaattatttttatttttagtagagacggggtttcaccattttggccaggctggtcttgaactcttgacctcgtgatccacccacctcgcctcccaaagtactgggattacaggcgtgagccactgagcccggccgaggctgtcttcttatgagtaaatggaattcccacaccaatctcattgctatcaggagaggtgcaatttccagataagctcatgaagtgtttagcatggaagttctcatttactggttggtggtattgttggtttatttatttagtctgttagtaaataaataaatgctggtctcaaactcctgggctcaagtgatcctcccaccttagcctccgtagtagctgggagtgcaggcatgtgccactgcactggtagtggtattgctatcatctgacttcaccttatttcctctagaccagtgccttacttgagacacagtgaagggaaggacccccagtccaaaggtctgatcaaagtcttgagggtccctttcccgatggagggagtcatggtgggcttactgtctgtcggaaaccgggaggctgagccgaccagatgacccctggagttctctggcaatgtgtccagttctggctgaggggcaccttggttgagtatggagccctgaaacagaaggcaggtgcattctgggcacccccctgcccaaggcttcaattcctgtggtctctctatgctcagcctggaaagcttgggccacctctctcctcacccctggacagaccaagtcctgctgactccgctgtctacaccacctcatcctccttccctcccctgctgtctgcagggcctccatctcctctggtcttttgtcctccagtcctgcaccctgccactctcctgctcatctgtctgaaattgtcatggagggagtaagggccaagcctggccatcacggtccttcttcatggcaccagtttaca

SEQ ID NO: 89 - DNA, 4001 bp, Homo sapiens
  source 1..4001: mol_type = genomic DNA
atgtcatttgtctgatttctgtgagcattttgggcatattcagtgtgacaactcaggtctcttgaccccagttccttgcttatcctacttctttcaaagtctggatagattcacaaagctgaaaaacattgcactacctcagttctcattctttctgggttccattgttaggcagttccttggttatcaagaaaagatctacaacgtaatttcaaaagcccctaagaaaattaatgatagttttgaatgaatcttgaacatctgcgatgtgccagacactatgttaagcacattaccttcctttttgcagtagctttcaaactttagttatttgattactaccttcatgaattttgccaaatctacataccatctatgtagttgttaacttaatgtttgtctttatattgaccttaaagttctaactactttagctttatcctgggctataatactcatgaaatcatgatgtgctaattattctttaaatacatactatataaatgctatataaatagcattttatatttaactttaaatatcaaacatataaatggtgcttatataatatgtatttaaagtgtttatacattataatataatttatcttttataatttataaatactatttaaatatgtatttaacactattaataattatataactattaacattaaaattgttgtctaagcctctctaatataatctcatttaattactccctaactctaaagtactagtataatccctgaatatcccatattatatcactgagcccaagtttctttgcctataaagggtatattgtatcctaatatgtttttgttttctcacaatagagaaaacccaaagctcagttaaataacttaacccaggcctcacagcagtttagtggcattgctagaacccagatccaagtctggctgactccaaagccagtgcagctaaccatgccatctaatgacatgaaggaatgttcagcagagtaagttgcaccagatgagaacttaaaggtgagaccctacagggcacagattgatcaacttatatgattgtgtgtacaggggtgggtgagtttagaaaccacgtataagggagcaaggacactatatcctttatatttttaagtttttttttaaagtatcatatctagagagaagttgaaacagtagtgaagtgatcattccataaccttcgcctttattcaccactggttaacattcaccaatttgttgatctctcgctctgtagtgtttgtgtgtgtgtgttgttttggctgaaccatttacaaataagctgtagatatttgtgtcatttcacccctaaatactttagcatgtacaaagaacaaagacatcctcctacataattagataccattattatatccaagagtgtgaaattgatacagtaatattatttaagatgcagcccatatttaaaatttcccaacgtctccatagttcccttattgctatttttttcttccttccacaatctaaatcatttgttgcatttggttgtcagtttgtcttaatcccttttaatctagagaacaacccttctacctcttttgtctctcctgcattgacgtttttgaagattcccgatgagttttcatgattaaatttaggtcaatctgcccccttttttttttttttttttttggttaggaggggacatatttctttagtaatatggttatgcacaattacttgaaaaaacttatgcagactagtcagttaacagagcaaaaatccttaaaaccccaattactgaataagaaacagttcttcaattggcaaagtaaagagaattaattttgatctgtggtagctacacccaacctcccccaaatcctctgtatgacttgactgtattcccctaactatctcctgaaaaccagacttgaaatgaatataatgctgctttaccttatcttctgtcattgtactagttgggagtcctgtcaattccaggtcttgtgtgagtgtggcacatgcacctgactagctcctgcctgtttattcgcagctctgcctaggcctagttatgcattgcaggaggaggagcaaggagagggaggagttgggctacccagacctgagagtccaagtctaatcttctcagcggcagctgaatattgatgggaatctaatcaataagtgctttgcctcagcaacctgtggcttcaatacctttgttccactaggaaagaaaatgggagcaggtcccttaggcataagttacaaaccagaggcaatcacatttcaaggcatgtgttatgtggcagtaattgggagctggtgaaaagagaaatctttggagtcttttgtagaagatgagatatgctgagctgccgtgctgtgctgtggagcatgggaggcacactgcactctgctccacagataattagtgctgtggtgtttctgtttagagctgagctgtgtggccggagaccattgttgacttgaatggttacctggatgagctttggattgttagggacttgggtttgaatctcaacattttactggggagtataaccttaagcaagtggctcactgtgacttttctctgattagttttttatttttgcctgataaaatagaaataacacacattttgttttgttgagtggattaaatgagataatatgtgaaagtgtttggcatatctcaacatataataagtacttagcaaatagcggttgttattattattgcttcatatatgtcaagaagcacttgctggaggctttaacgtagagttctcaaataaacctgctcatatggcttgatcttgggcctaagtatcccttatgatatccaaaccctgtgggggttcaggagatatcaaatttattgagctttaagtgtgtgtggcaactggggccacagattgcactgaggctcaaatggaaccagttcccagcggaacaaaaatgtaacaagcagcaacacaatgaggggtgttatgaagaaacagggtgagaaaatgaaggattagaggagattcagatttcacctttttattttgccaccctgttttcatttctgtcacctctgcaaggtgacacacctgaaatgtttggtttgagaaggtaattcaatcaaactacaaacttttactgagtccagcacagttccatgctccgagatgactgagataaataagtcacagcctcctctttctaggtgtttataacatataagatgagacatatgtaatgcagacacaatacaataatttcatagtggaaaagcgtgagcaaatattaatagtaatgatggttaatctttactgaggtcttgccgggtgccaggtgctgctctcagcacttgacatggattaactcgtttagtcataacatgaggtgctctgtgtcatagtgccaagctttggggagcagctttgaagtcagactttaaagcgcccagttagccacgctgactttccgcatcagttttggctggtgatgagcacgccccattgccagagcggtccacagtcaggagcggttccatggcatttgccagtgacatgacattctgcaggaaagcgtctctgtgccgcgtcgtaaatgataccggtgtggtttaccaacctttgtcttttgaacctgccttttcagaaacggaatttgccttgagtgcgcgggcggccagtgagcctttaacttcttgtgctcagccccggcctgccggcgcggagcacggcggcaggaaaccattctcttctcccattgggagcgcgacccgccattcactagcgggccttcggggcaccctgatcagtttcctcaggttcagccaaggcctctgtggttctgctctttctcagaaatgaatgagtaatctctgcaccatctgggatatgctaaaactttatctccgtgcggggagggggtgcgattcatttacattgatgctctgattgcttttgcccgcggagaactgataagcgattccagcctttcaggagagcgtgtcttggcgc

SEQ ID NO: 90 - DNA, 4001 bp, Homo sapiens
  source 1..4001: mol_type = genomic DNA
ttgcatcaggcaaatgtaggcatagaattccactcacgaactgggagggcagagaaaaagacagtctcgggtaggattactaagaattgaccattctggaaacaaatacagtgcagtctgatctgtaccccagactccacggaagcccactggtggtggtggagggggtggtagtttgtgtcttcactgctgggtggctctttgagtctcttatctctcaggaccatgtaacctactggatctactcactgcatcctggccccatttcagtcttcgtttattaaatacagttctgctatccctaactcccgcgcaggggaagaaagtacttactctgaacccaacatataagggcgaacccaacatataagagacatttcttagtggggagaaccggtcatcttcaccttgcctggaataattcagccgggcgagacattttccatttctaccttggggcagctcaatttttatacctgaggccaacttctacgccattcccccccaccccaccacccaaaatcccttcaacctttcatatcttaaactcctttcctttcaggtcgggcagattaggcagcaggctgggagcttcagcaggagaagtgaccttgcctgcagcctcccagatgtctcagtcccagctaagcatcggtaccccggagggccctgccagtgcaatgctctgttaaatctttttccgtttagtaaaaatctcttctgtctcctctcatcattcactatctttgagagccttttcttctttagtctcataattttccatccctttcttattttcctacattttctttctttctgctttgtagagatgggatctcactatattgcccaaggctagtctcaaactccaggcctcatgtgatcctcctgccttggtttcccaaatgctggggttataggtgtgacccaccacacctggccccttatttttagttaaatttatcttggtaaaaaatgcctttcttgctgcgcgcagtggctcatgcctgtaatctcagcaccttgggaggccgaggccggcggatcgcctgaggtcgggagtcgagaccagcctgaccaacatagagaaaccccgtctctactaaaaatacaaaattagccgggcccggtggttcatgcctgtaatctcagctactcaggaggctgaggcaggagaatcgcttgaacccgggaggcggaggctgtggtgagctgcggtcgctccattgcactccagcctgggcaacaagagtaaaactcaatcagaaaaaaaagaaaagcctttctccctgcaaataatgcatatacaacactcaatattttagtatgtcctgcactcacgattttaaccctaataggaaggacaactggaattctttctccttagcatccacgaaccccatatgttataggacaaacagatggccagaccctggcctgtgcctccaaacacagaccttttcaaggtctaatgcttccagagaagtcttcacactggctctgagacccacacttgtaatccatacaaccaagcagcccgatcctctaaacactcaaccatctcgtcctcagtggcatatggttacctaaaataaacctgagaagcaaacacaacttctccgggtgactttcctatgataaggtgaacagaactgtatgacattcaaagagaactgatgaagcggcagcagtgcaggctagagacgaaatgaaaaggccttaagatagcttggtgacttaggagaaaatagaacaaggagattaccattaatatttgcctttctgagtgtatcccaaacaagcaaaaataaccagtaattatatcattcttaccttctgtgtccccatcgctgtaatctaattcaagtcttcaagaaagatctaggggtgattccagaaacagctttttcagccaacaaaaccttcaaaatgtccttctcagcagcaagttttctccacgtcctcaggaaggatccaaagtcttaccattacagcatctctgggtctcgctgggatcatgactatttataatctccgcaaagggtggagcctgaaagggcgacaagcccccagccccaccccaccccaccccacccccctgaagcaatcttgtgatctctcggaattctgtgttatcagaactacccccgcccccgccccacagacaataacgccctccctcccccctctcttaaaaccacatccttccactctctccaccctgggtttttttttttttttttttttcaaaagctcatccttactccttccctcttctaatagcagctagagcctcttagtttcacagacaaaatactaccagaaaagcctggagggagggcaaatacactattttatgctctcgcatctgcttgggattaaaacaatctacagagagattagaataaatctatccatacaaaacgaagtcaagtgggtgctactatcagagatttaaggttgttagaaagagtgttagtaagaattcttgatcctatgaatttataggctgggtctacaatcttttttttttcttttttttttgagacggagtacacaatcttaaaggggagggaaagacagcctgagagaagaaaatcagggaaataattttgccagtgttcctttcttcgcataatctcacctggagggaagaaaatctgaatttctaggagccagaccaactatcagggcagtaggaataagaggttcaactgtttatggaatacaatccatatcctttttttttttttttttttttttttgatatggagtctctctctgtcgcccaggctggagtgcagtggtgcgatctcggctcactgcaacctccgcctcccgggttcaagcaattctcctgcctcagcctcccgagtagctgggactataggcgcccgccaccacgccccgctaatttttgtatttttagtaaagacggggtttcaccatattggccaggctggtctcaaactcctgaccttgtgatctgcccgccttggcctcccaaagtgctgggattacaagtgtgagccaccgcgccgagccctatatttaataaggcatttgccatatcacccattctgaattctgcatcactttctgctagtctcaagcttatagcatctagaaatgcaataaataccgaattcaattaagcaggtatttgccgaatacacgctatgtatcaggtactctccaagaagctggtgattcttcagtgaataatagaaaaaaaggccgggcgcggtggctcacgcctgtaatcccagcactttgggaggccgaggtgggcggatcacgaggtcaggagatcaagaccatcctggctaacacggtgaaaccattctctactaaaaatacaaaaaaaaaaaaaaaaaaaaaaaaaattagccgggcatggtggcgggcgcctgcagtcccagctactcgggaggctgagacaggagaatggcgtgaacgcggcaggcggagcttgcagtgagccaagacagcgccactgcactccagcctgggcgacagagcaagaatccgtctcaaaaaaaaaaaaaaaaaagaataataggacaaaagagttctgacccacaagtcatttatagtgtttcccttcttccctttttataaagtataaaaagggactagggcaagtaattctcacataactggaaaagattattaatatcctattagttatggactgtgagtttcttgaaagaaaagtgttacataagtatctgattactaagctatacaagaaaactgaacaagctaagcaggagaagaaaataggaatgagaactgagggagggataatcggggagagagaagagtggttccaggttgagagtaaacaaagcaaaacaacttggtttgtgtttaaacagctgggtaagtgagagatagtatctaggcttttttggaggaggaatatcaactatcaaattcctaatgcccattcatttgtagcttgcaaccctttcagtactggaggtggcagggtgggt

SEQ ID NO: 91 - DNA, 4001 bp, Homo sapiens
  source 1..4001: mol_type = genomic DNA
ccggatttttttttttcttcttgagacggagtcactctgtcgccaggctggagtgcaagggcacgatcttggctcactacaacctccacctcctgggttcaagccattttcctgcctcagcctcccgagtagctgggattacaggtgtgcataaccacgcccggctaatttttgtatctttagcagacatggggtttctctatgttggccaggctggtttcaaactcctgacctcagtcgatccacctgccttggcctcctaaagtgctgggattacaggcatgagccaccaggccgggccggcattccagatttttcaggggattagtgcagcaaaggaatcaagaagggatgtaaaggcacagtgtgttctgggtacaataaggacttaggcattgcccagaacaggaggcgaaggagatagaaggagaggcaggagagataggtaaggccagagatcggataagagaggcaggaggttttgttcactctgaaaagggatttgaacttggcaattggggcaacagagacagtgacttcttgcttgagagatgagattggaccttcgaaaattgttctctgccctcgtcataaaggaaataagaggagcacgaagaccagtgagggtgatggtgatctggactgaagtggcagccgccacggagaatatcggatgaatgtgagagagttttggaggtcaaagcaccaatgttggaaactaactggataaacgaggagagcggcgcaggacaggaggaatcgagcctgacttctaccataggggtgactgggcgggtaattcattgaaataaggaagttaggaggaggagcaggtttggacatgctgatcactagagctgccacatccgggcggtaacgaacacctggatctgcagctccagagaagggcctgggtcagatgtcactgaagccctatggtggcggaaaggcgagaaatagtgggttgagattccaagtgcaatccactgcggctcctcgctcgccctccaggtggcagcacaaccctgcgcttccgaagcccgttttctgagccagacactctccacgctctgggtatttcggcttctctctccccacacgccgaccctaggtcgcgcactttctgcctggcagaatttggccgaggatccaaacccggagcagcctccagagagcgtgtcgttcacgcggccagcatatgctcagagacctcagaggctcagagacctcagggctggtggtgtggtcggttgtgaccacttgtccctcggaccggctccaggaaccaacctggggaatgtgtgtaggggaagggcgggatagacagtgcccggagcagggaggcgctgaaagacaggaccaagcagcccggccaccagacccgttgtgggaacggaatttcctggcccccagggccacactcgcgtgggaagcatgtcgcggactctttaaggcgtcatctccctgtctctccgcccccgcctgggacaggccgggacgcccgggacctgacatttggaggctcccaacgtgggagctaaaaatagcagccccgggttactttggggcattgctcctctcccaacccgcgcgccggctcgcgagccgtctcaggccgctggagtttccccggggcaagtacacctggcccgtcctctcctctcagaccccactgtccagacccgcagagtttaagatgcttctgcagcccgggatcctagctggtgggcggagtcctaacacgtgggtgggcggggccttttgttccagggactcttttctcaaaacttcccagtcggaggctggcgggaacccgagaggcgtgtctcgccagccacgcggaggggcgtggcctcattggcccgccccaccaactccagccaaactctaaaccccaggcggagggggcgtggccttctggggtgtgcgggctcctggccaatgggtgctgtgaagggcgtggcccgcgggggcaggagcgaggtggcgggggcttctcgcgtcttttcccccagccccgctccaccagatccgcgggagccccactgctctccgggtccttggcttgtggctgtgggtcccatcgggcccgccctcgcacgtcactccgggacccccgcggcctccgcaggttctgcgctccaggccggagtcagagactccaggatcggttctttcatcttcgccgcccctgcgcgtccagctcttctaagacgagatgccgtcgggcttccaacagataggctccgaagtaggattcatcatgagggggcggggcgggggggcacgggtcccgcttttcttgggctggggtcgcggttggggtcagctgggggtggttcctgcgcaggcgcagggggtgaaggtagggggctggctatttatacccggcctggacaacccgtgactgtgagattccaatcctaccaaaaggcagagtgggtctggagggcctttcggggcacaggcagcaagtggattctgcgagccagggttcacccccttgcctagtaggcggcgcggcgctccggaatcggggacaccctgccctcgatccgactcgggaaagcagatccaggcgggtcttgccctccgggagctgtccgtccgtcttcgctcacgggcagtgtttcgaggaccggaggctctccgtgggcccccacccccactcctggccgccctccaggacgctgaactttcccttggccccatggttggtggaggggcagaggggactgtcagccccccctcctccagctcaggtttccgcttggagacagtctgtgccgccagcgagcggccaccactgccaccgcccctcacactaccttcctgccctcctcccctgggcatggctctcccaggcagaacccctggacggccctccctgcacggaggttagagggggagggcaggccacgacgtagatagagaaggccacccctagatgaccgggatgtcctttctggaacagcacttcttggtcctgttgggggcctcctggagctggctgacagaacccccagaggggagggaagaggacagtggctgatgataataatgcacgtgttaatttatgaaaccagcactgtcaaggatattgttaacatgtgatgttgattttcacaacactctcacagataggtaggcaaggcaggcaacatcacccccatctcacagaggacactcaggttcggggcagggaagtgacttggccaaggtcacacaaatctgagctcttaaggccaagcctgtctcaaggtcacagaagaattttgagacaagttctcaaacatttctctgccttatggacccaaacatccagtttctcctttatgcccaggttgcagttcagctcctgtttacattgagatctttgtgcaattcctaatatggcccagtttccctcacccaacatgttgggtggagcccagtatcttcaggctccagctgggcccgggcccctagcggaaggaaaaaaatcatggttccatgtgacatgctgtgtctttgtgtctgcctgttcaggatggggaaccccctcagcagcgagtgactgggaccctggtccttgctgtgttctctgcggtgcttggctccctgcagtttgggtacaacattggggtcatcaatgcccctcagaaggtgagggcctgcagctggcagggtgggggtacccaaacgaggaggacaggtgtctcgggggtggtggaaaggggacggtctgcaggaaatctgtcctctgctgtcccccaggtgattgaacagagctacaatgagacgtggctggggaggcaggggcctgagggacccagctccatccctccaggcaccctcaccaccctctgggccctctccgtggccatcttttccgtgggcggcatgatttcctccttcctcattggtatcatctctcagtggcttggaaggttcgcagctggagggcaggggtgggggaaacaggaagggagccactgctgggtgccctcaccctcacagcc

SEQ ID NO: 92 - DNA, 4001 bp, Homo sapiens
  source 1..4001: mol_type = genomic DNA
gagacaggttctcagtatgttgcccaggctggtctccaactcctgggctcaagcaatccttggatgagaaccttggcctcctaaagtgctgggcttacaggcaggagccactgtgcccagccagagatttactattaatatttgggtatttgggaagaaaacacagtcctctctaccccagctatctctgactggggttacatcagccttgagtcagcctctgggtgggggggcaattttgggggtgcgctctgtcccttaccttgccagctccccagaacctctcctctttggtcacccatcgtcagggttggaagtgaggccccctcccaaatagagtgatgtgtagtatataatgagcagccaaagttggggttgggtgggaaatgggagacccttaggaggaggagtgccggcatccggtgtaccaggaccttagatgccctgtcccaggcagtcgcgtgacatttggggagggagggagaagggacgggaatttgaactttccacacgggtccgacgccctggccgatcgggttgcaacatgacctgggccaggggccagagcttgtgggccaaggttactccgcctccagccaatggggtcgcagctgggcggctgcggttgggggcaggcaacactcccctcacagtccccaccccagctcctgctcccccgcagggcggcaggtgccgatctgtagccagcggttctcagaaggggaggaggcgccgcggaggaagaggaatatttgcacagagaaaagatcaaggccggccgatccccctttctcccctacgggttttcggttgataatttatactggcccctcaactccggggaactttgctccagggcactgggccaggcctcgggttcgcgccctcctctgggacgcggggcgtggagcggcagcggggcgcggcctgtgcgctctcggtggcactaagcgggggtcccaggcagcgacgggcgctcagcctagtgtggcgacgtgggtaggcgaacccaggcctcccgggcaccggcgtaacctctgcgagctgcgcctcttggacatttctgggacatcctcctcccgccacccttaatgtagcagatcaagaagaggtgtctcagcaattgcggggaggtggacgtgagagtgggccagttccagtccactttggttatgaactgggacaatcaggggcgctgggagtatctcagggtccaggaatagggcgggcttgggccggggctgctggggagaaggccgaggcctgcagggggtcgggtggagtggctccagagtggagagtggggatgggcgcgggtggcctagggaccaggggcgcctggcatttttgtagggacggaggggctgggaccagagagaggggaccagaggtccaggtagaggtggggcgcaggcggtctgcgcttcggggaagagggaatgggggaggaatgggaatcagaaatgcgtgggtccgtcaggggccagcgggctgggccgctgtaggaggacctcggggctgggagaaagggggtgtccagggcgtgatcggagcctccgggagccgcaggaccgggctcgggtggggtgttgagggccccagcgcgcagtcctgccccggaccccccccccatccccacctggaagccaaggccgtcagggcccggaaccgccctcacctgtaggccaggctcaagcactcgaagaggcgtgtgagtgtggggtcgatactgcgcggcccgaagtcggagggccccacggacccctcctggtcgcgccgccggggcagcgagtcgctgtgcggcgacgacaccatgaagtgaccgctgtggatgacctgcgagcggagcaagccgcccgcgctgcgccggagactcgggtcctccgagtctgtgtccgagtccgagtctgggctgggcgcgacccgcgggacctgcaagcccgcggccagacctgccagcgcgccggccatggctgtcgccgccgcaaccgcctggtccctgctccgcgcagcgcggggaacagctcttggccagccgggcctcattaacatagccccgcccccacaccataggccgatcgggtgggcggggcctgggacggggcggggcttgtattagcataatccttacgccaggtgagaacccggtgctctggagctctggcgggttggccccatctcccttcccgtctttccttggagtcctgatagagccgcccctactttgctccaaagggatattaagaaaggtcaggccggacgccaggggctcttgcctgtaatcctagcactttaggaggccaaggcgggaggatggcttgaggtcaggagttcgagaccagcctgagcaatatggcaagacccccatctctaccaaaaaaatgcaaaaattagccggacatagtggagaaagcctgtaataccagctactggggaggctgaggtgggaggattccttgagcccaggagttcgacaatatggcaaaaccctgtctctaccaaacatacaaaaattatccaggcatggtggcgtgtgcatgtagtcctagctactccgaaggctgaagtgggagaatcgcttgagaccaagaggttgaggctgcagtgagcggagatcgcgccactgcactccagcctggatgacagagtgagaccctgtctccacaacaaaagggtcccctatcacacctgagcctgaaagcgcctcaggacctggggtttgttctttcaggggcaccctcctgtgaggccagagatcaccatctcccctcacctaactcctgcccttcacctccaacccccgccccccatattagctcttctgggtcctccacataggtagaagcccccaactctcaggggcctgatgtggtaagcccaaggtaatgcccctgaagggcctggcccacaggaggttgctaacacctctagttaacatggtggccaacacggtggccgggcgcggtggctctcgccttcaatcctagcactttgggaggccatggtgggtgggtcacttgaggccaggagttcgaaaccagcctggctaacatggagaaaccccttctctactaaaaatacaaaaattaccggatgtgacgatgcgtgcttggcttataatcccagctactcgggaggctgaggtggaagaatcacatgagcccaagaggtggaggttgcagtgagccgagatcgcgccctactgcactccaccctgggcaacagtgaaatcctgtttcaaaaacaaaataaaatacttaagaaaaaatatatattaaaaaaattgtgatcattacttcacctgcacagagaggcccaaggggagcctcgagggactgttactggtagggatgggtggctcactggaaggtggacatcccctaggcaggcatctacccaggagctctgagccccttctctcactgacactgaggtccagcttttgttcctgcttctttgtatttttatttatctatatacttctatttatttattgttttgagacggaggctcactctgtcacccaggctggagtgcaatggcacgatctcggctcactgcaacctctgcctccctggttcaagcgattctcctgccttagcctcctgagtagccgggattacaagtgcccgccaccatgcccagctaatttttgtatttttagtagagacgaggtttctccatgctggccaggctggtctcaaactcctgaccttaaagcgatttgcccacctcagcctctcaaagtgctgggattccaggcatgcgccaccgtgcccagcctatttatatatttttagagacagggtctcgctctgtcacccaggctggagtgcagaggcacggtcatagctcactgcagcctcaaactcctgggccaaagcgatcctcccacctcagcctccctagtagctgggactacaggctcatgccaccaaacctggctaatttttttttttttgtagagatgaaatctgcttcagcctcccaaagttttggga

SEQ ID NO: 93 - DNA, 4001 bp, Homo sapiens
  source 1..4001: mol_type = genomic DNA
cataaagcaaggtatatgctcacataaaacacatcccttcaaaatatggtgggtgcatagtctttaaaaaaatgaaaaccccaacactctttactttaaaatgggtacagcattttgctatgttattgtttaaggtctatatttttctaaaaatccacagttgcagatttgactcagctgtgtgctttcagatcttgtatgctaagctactcaggaactgttgtctatgggattgaacacttaacagctgtctctgggaatgtaaatttgattctttggactgctcatgggactctgtgacattttcctctccagtgacagcagatttgaatggcattaggctactgattaaataatacccaagtaagtttaatgtcaacttaggcatctattttgcatgtattttttctgaaataatctttctctaattgcgatcatattttcattatactttagtcttttaatatctttaaaatatgctcttttaaacaggtcttatctgaaccctttcgttggtaatctttttctgaattgtcatattagagtgtgagtgtgtgagaattaaggttgagaaataaattagcttaaatacaaaacagctatctacaaaatagatattcttaaccaaaataatatgtctttatactaccattttatatcgtgccaccaagataaaaggtcgcagttatttaagggaattaccttcttaagtttcatgaaatcactgtgagttattcttttctaactgaaagcataaattataaagagattgcacatggtcatttctactttatcctaattaagtggtctgagatttgaaaaaaaagtaatagacttacaatatttttatgacttattttttaaataaatatgtcagtaaaaacttccctggggaaaaaaaattcttgaggagaaaagagtgtttggggttttttttttcagttgtatggcactgcttgcaaatgaaaggattcgttgtgtgcctcacatgcccaaaaaggacagcacgagcattctaacttaaggactggtttaaatatgacctaatgaactaaatatatttctgtctggtattcttaggttactttacttctaaggttgtttgaatgcatttatccagctctcctatcaaatacatgccaggttaaccctgtgaaggttttcctgtatgattctgagaatttctttataagaacagggtcttgatagtgaaggttctttccccaaagtgcaaggccttctattttgattcaatatcaacataatgctttttttttttttgaaagtgacttaagtactaaagattatcctcaatagtaattctacttagacatattttggttgaagaaatttaaagagtttattttttgtaggagcactgtgctgtgatcatgaaatttttgcaaaactaacagaaaatgataaacagttaagaacttccagctagaacaataggctgtttcctttttttaagtcagtttgtcatccttgctgattgacagtctccaattgttttcagaattattagtctcacagtatttcagaattacgctgtataaaaaagtacattaaatttaaacttaataaaataagtttcgcaagctcagtcaagacagggaagtgagaatatgattattcttccaaatttactatttccaccagcggtctcagttcagcatctgagaatatgttaggggaaactcagcaagtcagttccttttgtttttctggcctctgacttacttggatgggaaatagccaaaaaaaaaaaaaatgctgaatatctcagatataggtaatgggtcttcaccagaacataaaaatagacccttattagccatatcagtaatgtgctgtgtgggggattttttttttctttcaattcctctggcaacaaaccacacactgggatctgacactgtagagtgctttctcttctctttttttgggggggggagggggtgtggttgcatatttaaactctcacgcatttatgtactgaggactgcagtgtaggactttcctgcagaataccatttgatcctattaagaattgtccaaatgttggagcatttgattgaaaaatccttcttagccattttaaaggtaagttgtatgatttttctttaaataaaaaagattaagggatttttcccatcacccagatgacgggaggtgacactactgtataaatactcctaagaagttatataggaggacaggaaaaaaggtagtaactgttttcttagtcactaggctttatctaaaaatcaggtttgcattattttcaagttatcaaccttcctatattttatgcttaaaccatacccaatggcatttcatgtcgtatgtataagttaatatttaaagtgatttataatccaatttggattattataaaactttcattttatacttaggctatttatacatgatgtttaaatgatgaagatcattatactgcagaccaatataatgaggttagatgttataaatgacttacagctcttatgtcaacatgttgtctaatatgcaattattaactattactttacagagtcattagcttgtttaattttcaatctgaattgcacaacttttggtatacctgacttgctctacatgatcaaatgcaatgtgtattttggatatgtgctttttaagttcttttctgtaatgaacaaagatattgtcattgttactgggaatagcaaagtatattacttttaaataatctgaaatttaattagtgtaaattagtagaataaaaataaagttaattttttttcgattcaggaagcttattacaaatagtactgaattattagatgatgatgccaagttgaagtaaaggagaaatcaatttggaggaattaagtaaaagcctcaaattaattatacattaactaaaattataaataactgtaaatggcataaattctctagagtacatcccagtttctgaaatcatcaagtagcatattgaaacctgttttgtaaagatttgcatgtactttttagtctttttctgtgtggtttgaagtcagtaatgtgtgttgaagctaaaaaatattgctttaaatattggttcacaaggaagccaaaatgaaatatttaaaaatgtttgttatatagaaagtccagtggtcctttggaatttacaagaaaataagaactaaagtagaaatcatttaatttcccagactcattagtgaaccagaacaataaaccaaacagagaaccatcagataatgggttagcctgggaagaatattaattacaactggaggtacttgcacagagtgtactgaatttggaatttaccttggaagttatagttgccaccctatttttttaaggaatttttttaaccttgtttactttaacacatttgaaccctgcatgtgtgtgtatttcctgtgtgtttcctgaaaaggaaagtttaagataattctgtaactatcagatacttaattctgtaagtatccttgcatctaatcagaagatgtttttgttcactggcccaagatgtgtctgaaacttgcaagtagcattgttctaaactagtagctgtctttccatttgcatattttaattccatacatatatgatcacatttcattataatctaactattttgcaaatactcatttctacttttgtaaaatatacacatttattagatttttaaaacacttggccaatatgaaataacactttactgttatggtttgaaatgatacttcatagtaatatttcaaaataacaataacattttctacaatataaatatatattattttacttcatttaatacttgcaaccttatttgatgtaagtaaactcattttagagaaaactgtatcacagacaaacttcaatactaacacccagaactgaatctacatttcttggcttcaaggtccagcatttttggcattacaaactttatagtgtctgtttatagtagaatttggggtgtggaaaaaagacattagcat

SEQ ID NO: 94 - DNA, 4001 bp, Homo sapiens
  source 1..4001: mol_type = genomic DNA
taccacaacttttccctctccctcaccacctatccctataagctcatgcccagcagggagtaaacaatttaatgataatgcatatatgattcgtggaaaggaaatacaggatttcctgttcttatgggacataatccatatttgaagataacagtgcttacaatgtcagaggcagtctatgatatttactgtgttcctagaaaatatgcatagctgtgttcactgaatttttaacatgccttcttctttgataaaataatttgtttaaaagttcctagttcaaataatagagaaaccttttacacatagggaacaacatttttttccttaattaaaaagttcacattctacaatatttggaaatatgtgaccctacttttcacgtaaagaccatctgacctaaagggccaactccttgccacctgattgtccctttctctgtcacatatcagacctctgactggctgagaggtagccatcagggctggccttccaggatctggaagatcaaaatctgctcatacaaactggcaaggagaacgtgaaaagcataaatgcaagaaatgagaaatctccgtggctggagtggtggcttatggatcctctctctttttcctgtcttagggacataatgcttctgggaatgtcctggtgtcatagcaatactctttagggaccatttctgtgctttctgtaactttgtttaagcaacaaaaaagcaaaaaagctttggacttgctcaaagggtggctaatctgatacagataaagtgtggatgaaactcatttttagcctgtaactatgttgtaggatggaaaagaaaacttgcttgtgggtcaaaattttgtgggttagactctcaaatgaatcagttattcaccatgtgtccattgtcaaaacttatccttttagttgcagtttcttctctggaaagtgagcctaaaatattaatttgaaagcttattttgggattagaaaaggagagtgaattagatggtatctacgtcgtaaagggtcaggcacgtgagcagtgctcagtacataggacctattattaaaatatgatgaaaatactggttatggtgatttgacaaaacaaccttcttttgtggatagtggttactacttgatacagaatagtttcatttaaagagtggtaactctagtctatcatttccctttaatgtgattctgtaaaagaaattttaattgaaaatatttaactatcaggagagaaatatgacttttccagtttctctgttcacttcgttttagtatagaattagagattcttaacatataaatcctagaaagtaatttagaaatcttgtcattatatcttcagagtaataactttcatcacagggtctgtttgggctgagagtagagctttattcatgtttaccagaagtcccaagcaaaaattttttaaaccaagtacattctacaacccttcaaaaagattcaaaaaacaataatttaaaccaaagaactctggcagtgcatgacattttctgcctcattaaatgaggcaaacaaaaaatgaccaaatgactactgaaaacatagaggagaaaaaatactaagaaagtttacagagactttccagtaacaaaagttactggaacaaaaatctgttttgttgactcaatagtaatcaatattcacttttatgttgttttatgcttaagacaggaaaacctgagccttcctctttactatcagagcaaatggtaatcatgaagtgaagcaaattgattattgattactgattacttgaatttctatgttttataagtagagcaaccatctgttaacaggaataacaggttttagcagggaaaggattaagggttttattttcctcaactgaggcccaaaatgagtgagatttttcaagttaactttcattccatttggctcaggtgtcagggatctatatttaacttgtgttaggcatgcgtccgaagagctggaaggctgaggatgtcaatggctttcagatgtcaggataaccttaaggatagatgaagggttgagagcctgtgcctcatttctgagttctcagctgctatgccgtggaaatcctgtttactttctgcatctgctcctgcaagactctggagccagtcttgaggtcctacatctccgaaagcaagctcttctagaagttggtgagcagaatgctttttgtttagaagtgtggctataaatgtctttgtagttgatgcactctcatctagtaagtaactattatttttgggctctctagaaaggtaattattatctgatataatagtttagtctgtgatgcttcttttaacatatttgtaagttttaaccaaatggttaaagaaatttgctttttaacccttaaacctcacatatccacaagtctctaaattccataggatgctatggatttctagttgcctagttcatgtcttttacttagaaaacgtcagaaaacccaaacttctcgtgacttcaaaaagtgtaattgtacctgaaacttcttttccttcagatttcttatttatgttttctgataggtttttaagattaatcttttcagaaggatgctctaaaaatctggccaatttgattatcctcttccaacttggaaaaaatatgtatttaaaatgagactagaatttgaatgaccttctttcatggaactctgaaaatgttttttggcttgctgtcacaactgacaaatatatttaagaaataacctaaaagaccaatcttaacaatttgactcctagacagaagaaaacaaaaatctttttatacataaaacttactaatcattagtgatcatagaaattttcttgagtgtgggtgccacatgtcttatttgtggggtgggggcagattttgttacatagcagaatttttattatatggtgtgtttcataatatggctacataagatcagacaacaccgccaggaatgacagagtcgttacttatacagaaagtcaacttaagttatgcatatttcgagacacaattaaggttgatacaaaaaaagaaaagcagttccttctgtttctccatgctattctgctattctacaagccctggaaaagaattagataggcttctttgcctttacaaaaaaggtcactttcttttattatttggggaatggttggttggtgctagactgaattctggggaaccatttaaggctaatacttccaaaggggtaaacttgttgacactccttgagagaactcacaattcagatgggatctctatctcaaagagaaataggcaacctatcttgttttcaggtgtaactgactaaataataaaagtggatttgtggggcaatatatgagacttctgtaatcctgacaagaggaaaggaaaaatattttcaaatgatgtactatgcaaatactaattatttggaatctgatacttttgtaaaggataatgataactgtgtaattaactgttctaagagttcaagaaagctaacacctgaggataataaaacagttttcacctggggaaaagataagctctggaactcaagaattacagtcaggcaaaaaaaaaaaaaaaaagagttttaaactgctgagaaatgattgaaaaagactgcttaaggttgtagagtgggatgattaaatggaaggaaatgtttcagagcaaagaaatataaagttcttgatgtgactgcggctcattttgcttttggcacaaattactgattaagatgactacttaaggaatttaatctgatattcttagtatttacaaacaaagcttcaggttaaattgttttgaaaaataaagaaatgctataaaatgagtgaagtgcacttcagataaaaattgtgaagtgtgtgcaacgcgcttcacaacaggttactttattgccacctgtgaggagcaagaggaggtactagaggaggggaatgaggttgggtttccatatcatgtgactagaatcctcagtcatgtctaatccacagtggttcaat

SEQ ID NO: 95 - DNA, 4001 bp, Homo sapiens
  source 1..4001: mol_type = genomic DNA
tccctcctcacccagccccattattctcaagcccttaaggccactctttgcacagaaccttatatttggaaacatctcctgaatccaacctcccctgcccactgggatgcccaagcgaggccctcacccgctgaattgtggctgacaccctgggcacaggaagcttgggcagagatgtctggaagctgtagagcatagggtgccggctggatagaaggcggatacacatctgggggtacagagcagagtgctggggtgggagccagtggaaaaaatggaaaccaaccagcaactccctgagaccctcagagccatcccagcccctccaggacaaaccacacaccaggcaccagtgccctgtctgcctctcccgtctagctcagaaccccaaaggagtccaaagagtctcaacccaggtgcctgaacccctccactggctgctgctcacagcccagatcctggtcaagttgctggtcttgccatgcatctcaaacatccacccatggtagcagagaagcagcttcagggtttggcggaagaagaagatgcccgtcacccagacgcccgtggagaagatggccatgctgagaagtgcccgggtctgcggggtctggtaggggccacacctattggagaggggcaagggctgcagggggtcctcttgaggccagcctcgggtggctgtgaactgtctcaggggagacaccaaggggaagaacctccctgggcacactcatgtctgactgcatcaaatgagcggatgtagggctaagacaatggttgtcataggggagggctgccccgtgactgccaatggctgctgtcctgtatctgcccagctcaggaacctcagccttgggagctgggcccagccctccctggtggcacctgtgcccttccagtttcactccttacccctgagggaggcatctctggatgcaactgaccagccccaaggagatgtccacgttgcagaaggaggaacccactgttgccatgatgacgaccagccagctggtggggctgccagggtacacgcccctgaggatgccattctgtgggcagaaacaggcgcccttatggagccggaaggcgggaggttagcctgcctgtgaagtcttgggaactggctcctggtcttggcctggaaggctttctctcctgatgccctgctgtgctccttcctccgccacacccacagctccctgtgtccgaccacatcctgtgcacccctctggtgtctgtacttccacaacctgtaccgggcagaagtgccagccaaggaggcagtgagggaccctaacaaagcgcttaacagctgacagccggcctctgcctacgctctgggagaagcacctgtgcgcaccttgatgcggatcaggcgtttcttccaggagttgatcccagacaggtagacgtgtttcagggcctcccgactgagccggaagtcgaccccgtctggggtcaccgtgaactggaaggccacggcctggtgagcttccgccatcctgggggttggtcggcacctaggacgggggcagatgggtgcgcgggcgcgcttaggccggccccgccgccagccgcgccgagacgcccccagccagtccgcgacccctcgcgccccccaccccgcgactagcggctgcccccggcccgcgccccccgccaggccaaccgccgccaaatcctcgcgccagccttccgggtgggcacagccactgtggtgcaggggatttgggccttgaaagctccaggagccccaaggacggcgaagggcagcctggagttggggggcgtcagcgagggcgcgggcaggggagagaggcgcccctggaagtggagccggcctggacagaggcagggttcgggagcccgcccttccccacccgagcgcgcgggccccagcgccgtcctcgcccctgtgcagggctgagggcccagacccccgcgggccgccctgtgccggcccctactcacagctcgggttcactcctgtccgtgcgtggggccagccactctggcccgtgtccccacgtccttcaggcctagccacccccgccccgccctcaccaggaacctgacacctactcccaaatcggggtcggggaaaaacgtggggcgagaaccagaacccacccaccttccgcggtgggaattgcagggcgcggctgggtttctgtgcggtgaaggttgctcaaaggtctgggagcctggggggccccagagtctcgtgaggatggtggctttagagctaaaaatagccaaatgtagggaaaaggtcaccgatgtgtcagctgctgcctgttctgggatcgctggtgctccgggcggcatccccacctcagggtcgtcttcatacccccatcactctcctcctccccaagtcctagtatttattattctaagctgcagccaggggactggggagggatgggggaccccaactggcagcatgaacgctaaccactttcaatcccagagcactgaagagtccctttgtgtgtactgctcggtttggtactttctgctgctgctcagcagggccaggacagggtgggaagaagaagcttgtttattgtgttacatacacagcacggggctctggcctgccagccatggggacctactcaaactcaggaacaggccggtctcctgaacctcagtttcacttgggggctcagcccagtcgccagggccttctgctcgttgttcctccctccaaggtcctgccctggaggctccaggagaaatccaaggagtgggagggtggagtcaggatgaggagtggacactggtcagctgccccttctgctggaagtagaactggaaccgagactgggcatagtcctagggaaggaaacccaccccacacagtggtgagaagacgtgggaccagcattctacatccacctcaggctgcccacagccacccgggactccccaggcctcctggaagagtgtggctgctgctgctccccagccccccagctagcattcccatccccagggtcacttaccaagtaaccaaattctatggtggacatggatgcctggaggatggaccacagaccccagaagaaatgggatgccagagcatacctggggggagggcaggaaaaggaggggcattcaagagcatgtctagcactgcttggatcccctcacccaggttggccaatccctacagttttaacttctcccccactgtcctaactccactctccacagccaccttccctgcagcttgataaatctgcctcttccccaatccctctttcctctgctctgccctaccccaccctgcccctccttcctcaccgactgacttctaccagcaaatcttcttccagttttctctgctcctcttgggagagggtctcacctttctttgcctctgccaggtaatgacgaataaaatgcaactacgatcaatggccaagagtcaggaattggggagactgtggagtatttcccaggtaacatcctttccggccattccttccagactccacctccctccttcctcacaggatgggtcctgctccccagcctctggcccacatacctgctgttcttgagtggggtagtctgtgggccttgctttgtagaaaggccattcctcgtgagtataatcataaacccactcacaaaaatggttcccaatgtcaaagcccctgggagaataaggtggacattcagtccccaaatgccctgggcagctggcctgttttcaagagccctatgggaacagatctatgggaagccatctttccagcctcacctatagttataactgctgtactcgaagtccaccagcatgaggctgtcagcattttctggctctgagagcagcaagatgttccctgggggaatggggtgaggttctgctcactccagagccctctggctcttccatcttgggttaggagactcagatgccttctcctaccttcctggatgtcattgtggcagaagacgactggcgatggggtagactctag

SEQ ID NO: 96 - DNA, 4001 bp, Homo sapiens
  source 1..4001: mol_type = genomic DNA
caggctggagtgcagtggcgcgatcttggctcactgcaagctccacctcccaggttcacgccattctcctgcctcagcctctcccagtagctgggactacaagcacccaccaccacacctggctaattttttgtatttttagtagagacagggtttcaccgtggtcttgatctcctgaccttgtgatccacctgccttggcctcccaaagtgctgagattacaagcgtgagccactgcgcccagcccatggtgtcttataagtgtcttatataagggtaccattttttttttttttgagacggagtctcgctctgttgcccaggctggagtgcagtggtgccatcttggctcagtgcaagctccgcctcctgggttcacgccatcctcctgcctcagcctcccaagcagctaggactacgggcgcccgccaccatgcctggctagtttttgtattttgtttagtagagacactgtttcaccgtgttagccaggatggtctcgatcttctgacctcgtgatccacctgcctcggcctcccaaggtgctgggatcacaggcgtgagccactgcacctggccataagggtgccatttttaatcttttatataacatttttactataccttttctatgtttagacaagtttagatacacaaagacttcccattgtattacagttgcctacagtattcagtatggtaacatgttgtaaaggcatgcggcctaggagcaacagggtaagcagcatagcccaggtctgtagtaggctgcaccatctacctttgtgtaaatgcactctagaatctttgcacaacaaaatcgcctaacaatgcacttctcagaatgtatccccttcattaagcaacccatgcctgtacttggtaagtggattatcccaaaagcctcctggtttcctgccagctgctctgtcccctgaccacacctgcctcacccagcagtgaattcgacatgggcaggaataggaccttgtttaccactgaatccccagcacagggccaagtctatggtgggtgttcaacagacactggttcctggccgggcacggtggctcacgcctgaaatcctagcactttggaaagtcgaggtgggaggatcacttaagctcaggagttcgagaccagcctgggcaacatagtgagaccctatctcatgggaaaaaaaagaaaaaaacaaaaacagatgaagacactggttcctaaatgaaggtccagaactgaatctaattcacttcaattacagtaattgagggcctacttatacttttcataaaaattattttttatggctgggtgcggtggctcacacctataatccctgcacgttgggaggctgaggcaggtggatcacctgaggtcaggaattcgagaccagcctggccaacatggtaaaaccctgcctctactaaaaatacaaaaaattagccgggcgtgatggcgggcgcctgttgtcccagctactcaggagctgaggtacgagaatcgcttgaacccaggaggcagaggttgtagtgagtcgagatcgtgccactgcactccagcctgggcaacaagagcgaaactccgccttaaaaacaaagaataattattttgtatttactcttaacatgattttagcagccgagccaacctcagtgaaggagtgtctgttgctacaggtcatcctgcgtgccctcttgcagacatgaccacctggcgcctcaagacttcaggcctgggcctgcagcccctttccatgctgacaagaagctgcggggcccgtgcagggttgtgcctcagccaagaggggtgcctcacacatgcccctgcatgggaggaggtgggctcactctccagcagacagacaaacagaaatggccccagggaacaggtgaaggttctgaggcaagtatttattccaagcaaacacgtgggcgtggacagctatctcagagcctacgtcactcagcagagtcacacatttcaaagcaggtcacacaagtccctcatgaacagtcttccagcttcagcccttctcttgccacctctgtggtctgagctgggggagggggagggcctccgagttcccaaacaaaacacaggtacctaagggctgagagaaaattacacagccttatttttattagtatctatcaagaaaaaaaaaaatcaaaattcccttcctgctccaaccccacctccaccacctgggccctcagggagcagcatctgcccagcgaggacctgctgccggagctgtccttggagcctgggcacctgtgcacggccacccctcatgcctgtgagctgccacaggtcccttcctccacagtgtggggacgaaagggcagctggcatttctccaaccttcagctgtaggccttgggaacttggaaaagatcagcaatgtccagcagggctttgcggatgtggtttccaaagcgctgggcgttctgtgggagccaagagttcagatgcaccttcagatatgtacccacccctctgttcccacaagcaaggatgcctccccagccaaagacacctgtgttcctgggcagccccaggtggcaccccttcctcaggcctgagctgtggcaggagactcaccgtctctgagcttgagaacttgctggagatgtggaagaagatcgtgttctcgcctgcaatcatgtaggaaactccatagccatcatctgctacctgagggcaacaagaagggtgaagaaggcataaatcaaaccttgaagatctgaaagccaagctgatattgtacttgttcttcccacaagagttgcaggaacgggccccagccctagacactgcatttttcctgaggaccacccccaggaaactgtgaaaaccaaagaatgctgcccagaccccgccaagggatctgggacctcccagtttccctcagggggcttattctctagatcactccgtctctctccagagggttctctagccttctgacaagctagcccatgtgaaggtttctgtgggagacagctgcactctcttggataagtagacttcagaagcacccagtgcactgcccagtaccctggaaaccagccccacagtcatgcttcagtaccccggaaaccacggtcatgcttcagcagggaggtatttgggatgggtgggtcccaggtgcttggctgcctgagtccatagctaacattctgcttccatgcaggtctggggttatgctctcctctaagcaaggtgctcctgcctctcgtctgacctcagggcctgcactcgccactgaaaccctcagggccctggtgctgcccacccacccccttcaggagcactcacagggccaaagccacctccagcgcccaggtgattggggtgctgctctgggtcgaacatgcggatctgggattgggggatctggctggtggagagacgccagggttccgagagcacctgcaatggaggccacagctggtcagaggccacatacccaaagcacctgtccaacacgtggtgtctgcgacctactctgagccagacacggttctgctgagggtgcaggagctccctggccaccaccccgtggtcccctgtgcatcatgccacataaggcttgtgtgagctgcccagccctccctctgctctgcctctcccccacatagcctcactagtctcagtagtgctgtcaccacctgacagatcatatatttgtctgttccctgtcacaaggacaccaacaccacaaagaggatgttgctcctgctcagtcccaggaccacaggcccaggtggggtgtgggcgggaaagctggcccagcactccagggagcttcaggggagcaagccaggaaaacaggccgtgctccatcatgagatggcccaagccctgcgtgtaggagggcagtgggacaaaggacacacccaacaacggtgctgacctcagcaaggaaaggagagctgactcctaggtacttggagaccaagtaaaggcagaagaggtgcctgtcgatccctgccccg

SEQ ID NO: 97 - DNA, 4001 bp, Homo sapiens
  source 1..4001: mol_type = genomic DNA
aacccgggaggcagaggttgtagtgagccgaggtcatgacattgcactccagtctggacaacaagagcaaaaactccatctcaaaaaaaagaaagaaagaaaaatttctaaagtgatgtaaacattttttacaagtctgtaaagtattgactgaaaaccagtgtagtacaaatgtatatagaagtgttgggttgtaaaggagtgatgagagtggaggctgactgaagaaactcttcaggtgagctaaaaataataaaaacccttactagatccaaagagtcgggttgcactgctacccaaagttaagaaaatgcccccgccccccttttcttaagacagaccaagactctgtctccaaggctgaagtgcagtgttgccatcatggctcactgcagcctcaacctcccaggctcaagagatcctcccacctcagcctcctcagcagctgaaaatggccttggtggctcctctctccttaagatcttaggatttgtaccaactcatcagtagttttacaatctgcacagtacttgtacatctcatttcatccccacaccaaccctaccaggcaaggatcactggcctcatttaacaagataaagatgaggatactcctagttttgctgagtcctttaagacggctcagttgaaaaagtaatagtgccagggcttaaaccctcatctctaacaccaaagcctaagctcttcctgttaacagtacataaaattcaaacccaaaccacaaaattgctggggaaaaaaaaaaaaactaaaacttctttaacccaccaactacacagaggatgatcttccgaagttctcttccttacaactcatcacatcttttgaatggtattttaataactagcattctatttattttctattcttggtccattgtaccaccttacgagaagttaagagtaatctaaaatttaaggtaggtctctaaactatcaacaaaacactattaatgtacctaaaatgaaaacttgcgaattccagggctaaatgattcaaacaaatgttaatagaaaaggagggcaccaatctgaccattagccaatttaaaaatttacgcattacttgttatacataatcagggcaactttaagacgccattccaggcagatttggggtagtaggcggctggcttttagctaactctgcttagctgcggggtaaatgtcaaaggtcagccccatggcgaatctccctaagccccctgtcgcaaccgaagccgatggaatcaacaagacaaagatgctcctgacccactggctagaatcgtgaagaaaacgctaatccatgtcaaatgcacataaggaaatttttccatgcagagaccagacaggggaaaaggggaagggagaaggagaaaatacagggttcgacccgggccatcggaagacaaccaagcgctgacttgggggtaagccctggactaggggcctgtagaccaggttcgagcgctcacactcgctaactgcggagccttggccagcctcagtctccgagtctacaaaacaaggataatcataaagggctcccacccgcctcaccgagggctgtgaggctcttccgagacagaggacatggagccatttcgtaaaccggagcgcacggtacagacaagaaactgttatccggcccagcagctgcgagacccccgagcggaatgcaagtggaccccaggcaaagaggacagcaggggacggagcagatctggccaagttgtcccgtccccgcccagaatcagccccgcggccccacagaaccgcctgcgtgtcacttcccccccacccaaccccgccagggccagcctccgctccgacctccccgccttccgtcccagcccctcgagccccaactcccctccccgacgcgaccctcagactcacggcctgaggggctcctccggcaaaacagcggctggctcggaccctccctcagagcacactaaccagcagcacccgggaccgccagctactcgccggatgtacgtcacacccctccccgggacttccgggcgcgtaatgggccccgccccctcacagcgttacgcctctctgccccggtgccgtcccaccagcgactcgggccgcggaggggcgggcataaggcgtgacaaagcgcgctcggggtctggccccgcctcggccccgcctgctcccgtctggctagctgaccgcgcgagacgtggcagcgccggaaccctgggtccggggctgggtcctggagtccctgagcggcctggtacactcgggatcgcggcttcccattccagacgcaccaggaaagtcagaagtgggtaccgacggtgacataggtcctgggaaagatgagttgaccttggggccctccacttgaggtggatccctccaagcttttgctttttttcttccttgctttttttttttttttttttttttttgaggcggagtctcactttgtcgcccaggctggagtgcagtggcgcgatctcggctcactgcaacctccacctcccggattcaagcgattctcatacctcggcctcccgagtagctggaactacaggcgcatgtcaccacacctggctaatttttttgtatttttacagagacaggtttcaccgtgttggccaggctggtctcgaacttctgacctcaagtgatccgctggcctcggccttccaaaatgctgggatgagaggcgtgagccaccgcgcccagctgcttttgctttgagtcgcacttccttgccctggggtggcctatgttttagaaaaacagaaaaacagctaatatttattgtgcacttatgttccaggcagagcactgggtactttgcacgctttgtctaatttcaccgtcattggagtcctgtgactccagatgctagtattatccttattgacaaatgagagagcaatgttcagaataacctaccatgcccaagttcacacagctaagtggcaaagcaacgtgttcaccccagtatatttcgcatgcattttctcatttaattctcaggacagtcccacgagattggtactattattactctcattttacaaataaaacagtctaatggaaagatgaatgacgtctgtgggttcatgaagatctcaattctcctttagtaaaatttaaatttaacataatcccaataaaagtactaataggattaataggtcgtgtgtggtttcgttctttttttttaattaccctatttgttcctaaaattcgtatagaaaaccaaacaaaactagtccagaaacaggctttcaaaatgaagaacaaggaggtgggaactaaccctgttggatattaaaacatattatagagcctaagttattaaaactggtgtgcgtgcacatgcacaaacacacacacacaaaaaacacaatggaacagagcaaagtacagaaatgactaaagtacatgcagaaataaaactaaagtagcacctgggactccagcaatactcagggagctgagtgggcaagctcaccaccctctaccctccgctcatcctggggtcttagatgttgaatttgaaccctgtcctgtcacagaaaggtgtcaggccacttctgaggcttctcaccagctttagctaaaccagggacaatgtgtccatggggcttggaaccccttgtgcatcctggagacacagatgcaagcccagtgcaagccaacacattggattactggctttgttgagagcctggctgcacgaggtggaggtgtctgaaccacaggatggcttgggaggctggggaaggagggaacatgcaggcagattctcagaaaagccgcaaactctacttacctaaaggcttgagcaagttgacgaatgccccagaccaccatgtacccactgtaaaatgggtgaaacaggcattgttgtgcacatagtttttccagagtactgttttcaataaatgattctgttatgtgttgttaaataaattaataaataataaaaggggttgagacaattgggtagtcaaaaaa

SEQ ID NO: 98 - DNA, 14457 bp, Homo sapiens
  source 1..14457: mol_type = genomic DNA
ctcacctcgccaccctgcagcagcaccatctggttcttcaggtctatccccactactagcccctgccggaagttgtccttgaaagtcaccgagtaagaaatgaatgtctttttggcgaaccctggggaagggacacagaagaggtcatagggcagagccaggctgcccacgggaagccagagtgactcagaacctttcacgagctgcaaaccaacaggggtgcccttgaccaaagcgtcgggccattaggtatgcctggggtacccctacttcctaaaatgccaatctgggcctcagaccgtcagaaaagctcaagcttttacttctagggtctcattgtggtggctacttaagaccaaagaccttgagcttctccagaacttgtctgacagtggaatgcaggcacttggccctaaatggagaaaaccacccccagccctaaaatcaagccccagagcagagacaagacccccctcacagagacagccccagacgggagcacattacctgtctccacggaggctcggagagcagccacattgtggtggaaggagtccttcatgtccaccagcatgaaggggacgttcagggcctgcagctggctggctgctgcgatcccgccaaagcccccacccacaatcaccacgtgcagagctcccgattccaccgagacctgggaccccatctcaaatcaggcactgctgggaagaaagaggagagcatatcagagtcagggaggctgggagggctgggaggacccacagtgagagaggcctcgatcatgaattgacctttttagatgaggtaaatctttgattaatgaaactccaattttataaaatttgaattaaattcagaaggaacaagggggtatcccacttccagcccattccccaaacaccaagttcccttctccagaggcatccactgttacgtaacaagtttggatgactccttccagagatagtttatgcaactataatcacgattattttttgcacccattaaacaaagtgttctgcacatttgttttttacttgatatattccatcaaagacttttgggagagattgcagtggggcaggaggaaaaaaaagacttttgaagaggggaataatgttcactcaacaacatatatgaattgaactcctgctttttgacatgctctgtgttgtgtatcctgtggtcagacaggcagatatggtccctgcccttataagggcagaccttacaaagtcaaagaaccaggtgggcaatgtcaatgatgtgagaagctgggaaaaggtgcaataacaagaaacagtgggcacacctgtggggaaccagcaagcataggccacatctaaaggaacaacaaaaaacattctttaagcactgcactaaacaaacaccgcaaccagattcagcccagctcacaggtcaccaacttaaggtccaggtctaaggaggtcaattagcaggttacgacaagaatcctgcaaaattctgcttctagggtttgaatatgtctcccaaaaaggaacagtgttaagaggtggggcctaatcggaggtgtttgggtcatacggtctccaccctcatgaatggattaataccaattataaaagggctttgaggctgcaagttcaagctcttcctctctctcgctctctcttttgccttccaccatgggatgactcagcatgaaggccctcaccagacactgggcccttgatcttggacttcccagcctccagaaccatgagccaatacatttctgttcattataattacctagtctgtagtattctgctgtagcagcagaaaacagactaagacaaatgtggtgtgaactaagactgtgatcatggagtaggaaggaggggccacaaccatatgctacaatgcctggctaattttttaattacttgtagagatggggctgggtgtagcagctcacgcctgtaatcccagcactttgggaggctgaggaaggaggatcttttgagcccaggagttccagaccagcctgggtaacatagagagactctgtctctacaaaaaaaaaaaaaaaaaaaaaaaaaaaaaaaaaagccaggcgtggtgatgggcacctataggccccgctaattgggaggccgaggtgggaggatcacttgagccagggaggtcaaggctgcagtgagccgtaactatgccactgcacaccagcctgggccacagagtgagaccccatctcaaaaaaataaaacaataaaaataataaatattggtagagatgggggtctccttttgttgcccaggctggtctcaaactcctgggctcaagtgatcctcccacctcaacctcccaaagtgctgggattacaggcatgaaccactgcacccagtccccatttctttgtatgacctcaagatggtatataagcttctgtacctcttttgggggtcgggtcttccttctgaaggctcctgtgtatacacacattaaataaatttatatttttatatttgtataaagttgccttttatccaactcaacagccttgggccctacagcagcaaagttgcctgggctccccaagccagcctggaaggctggtaaacagggttacctgtgcatcccagagtctgccccagacagaggagggcccaggggctggcctggggctgcaccagaggccttgcctccagcccagcagaaccccaaattaggatgcttgcagtgtgggcctcaggcaggcagagggctgctggtgtgggaggagccgcacagcccattgaggggcactcttgactgcagctgagaccctgagaggtgacaagcaatcaaccctcctcctggcctgtgctgagccctaaagagaggcagaaataagccaggctgctggggggagatgctgagaaaaagaaacaaaactcacactatacagaaagcagccccagaggctcaacagctgctgtgggcttcaggaagggggactggagcgggtgtcagcgaaggaggaagtagactctgaggtcaaccttgaaggatagacatcaagccactgaacagcctcataaggaagtgtgttccagctcatctgggaccctcaggccgctggaaggccactgcactgccctgatcatctttgcgggtctggggcacaaccccttccctctctaggcctcaatttaccccctataatgggaggggactataaggcccaaccagctataatatcctgagctgtcctcgtacacactggtttccaagacatggtggtggttccccaagacggtccccaaatcatcccacacttatggggcagcttttggttagaactccgggcactgttcccattggtgttcaaatgcccactgcctaacttctcccctgggggcggacagtagagcggacggagggcactgacacaatggattggagggaatttaaagttgtttctcgtgggtcaaatggtccccaaagcattgaggcttgaacatacccatttcacagatgaggaggctgagccctcgtagagattaggacccaagacaaccccccggcaggcagggccaccctggctcctctaagccctcagaagggttgtctagagctaatcccaagcgtacctcaggctgaatctgccaactgggccagaagggaatgtctcctagggaccaggacccaggagggcccacaggcccagccagaggtagcagtgcacaccaggagctgcgcagggcctgagaatgtaggcagtgatgaggcaggccgtgggtggcgtgtgtttcaccagcccctggcagcgtgcaaccctctgtgctgggtcttacccttggggctgtcagccagggatgaggtcaagaaaacacccagaaagccccacttccaagttagctgggcatggcccctgagaggggctacgcagctggccggggatagggacaggggagtccactgtgcgtcaggtcacactggagcacccagggacactcaaaaaggacagcctagagccctgaaaaagagctgagatcagggggccttcctagcagaggaaacaacttgggccagaggcccagtggcaggaagggaggaggcaggtagagttcagggcacctgtggcagaaggtggagaggcttagaggacaggtgacaggggccgacagacagacagcctgccatgccagggcagctttgacaatcaggtggacatacaagcctgctccgggcccttcatggggtttcctccagggaaagaaagtctagtgaccccagagtggacctcaggggcaatgagttgaccaaaggagccaagcccttgacccccaggcccctagtatcatcaacctgcttccccgaaagggcaaactcttgcctcacccaaggaaggcaaattgcttatctggggttccctgtgaagggcctggggcctgaggggacacccagaccagagcagggccagggaggggaggcacttttcctcttcaggctgcctgaggcctctcctgcaatctgctctctctctcttctctctcctctactgacccctgccacgcttacttgtggaatagggaggccagtgactctccagcagatttcaccctgcctctttggcagccccttgcccaatactcttggacatgccgactctctttggctctgccagtgtcggttcttgcctggccccagaaaacagtgaactcccctctgcttggccatgatctacttggccagacacctaaagcaagggcttaagccatggagctgcatttccagccacacaccctgtaggagatgaaaatgcacgtatggaattagtggatgcccagttgtatgttctaacatagcagtagtgctgtgcaaacctccacctgtgtgacactccatcaatcaatggaaactgccaccaatgagaggctacaggataaaatattataaacccatacaatgcagtactgtgtagccagttgtttttaaagggtgagtgatgttttgttttgtttttgagatggagtctcactctattgcacaggctggagtacagtggctcaatttcagctcactgactgcaacatctgcctcctgagttcaagcaattctcctgcctcagcctcccaggtagctggaactacaggcatgtgccatcatgcccggctaattgttttttgtatttttagtagagtcagggtttcgccgtgtcggccaggctggtctcgaactcctgacctcaagtgatcggcctgcctcgacctcccaaagtgctgggattataggtgcgagccactgtgcccagcctgtgaatgacgtttttatacaaaaagtctgtccatgaaaaagtcaggcaagggctgggtgtggtggctcctgcctataatcctagcacttcaggaggcaaaggcaggaggactgcttgagcccaggagtttgaggctgcagtgagtaactttacctttgctccctcccacaacacacacacacaaagacacacatacacaatctgatttacacaaaacatgttttcaaggttcatccaagttgtagcatggatcagtgcttcataccactgcacttcggcctgggcaacaatgtgagaccctgtctcaaaagtaaaaaaaagaaaagaaaaagtcaggcaatatttatttatttatttacttatttttaattttcttagggacagggtcttgctctatcacccaggccagaatgcagtggcatgatcataactcattataaccttgaactcctgagctggctaatttttttttttttttttgtagagatgagggtctcactatgttgcctaggctgatcttgaactcctagcccctcaagggatcctcctgccttagcctcccgaagcactggaattacaggggtaagccactgtgatttcgagctgtagaaaagattcaatgagatcatttacgtaaagcattttagcatggtgcctggcacagagtagcaatgaatacacagcagccacgttttatatatatatatataaaatatatgttgcagattagtttgcccagtcccaaaaagtcaggccacagaaccacatgaaagatctaatcccatttatggccaataagacacatccacctcggtatgtatacacatatacaggtaatgattacagcagttagcatctgttgcacttttgcccatatcttcgatttaaggtatgagttttttctccttattttggaaaaaagttcaacaacttgcccaagttcaattctcaacacatcaaacctgctgtctgaaccaaagtgctaatagtggtcatagataatgttttcattttcttccttaagcttttgtattgtttgaatttttttcaaacaagcatatgtcatttttgcagaaataatgtctttaataataggacagaccgcttaccatctaattttttaatcaaaatagaattcacatatacaatttaccacttaagatgtaagttcacagtgagctatgattgcaccactgcactccagcctagatgagaccccatcttttaaaaaaaaaaaatgagatgtaggctgactcacacctctaatcccaacactttgggaggccaatgcaggcagattgcttgagctccggagttccagaccagcctgggcaatatggtgaaaccccatctctacaaaaaatacaaaaattagccaggcatggtggcacatgcctgtggtccctgatatcaggaggctgaggtgggaggatcgcttgagcctaggaggtcgaggttgcagtgaggcatgatcatgccactgcactctagcctgggcgccagagcaagactctgtctcaaaaaaaaattttttttaatttaaatttcaaaaaaagatgtaagttcagtgggttttagtgtattcaccaggttgtgcaaccatcacaactatctaattccagaacacttccatcacccccaaaataaatcctgtaccccacctaccctagccctgggtagctactaatgttctttctgtctctatagatttgtctgttctagacaattcatgtagatggaattatacactatgtggctttttgtttgcgacgtcttccactgatcatcatgttttcaaggttcatccgagttgtagcatgttatcagtgcttcattccttttcctggctgaaaaacagtccattactggtgtatgaatagaccacattttgtttatccactcatcagctgacgggcacttgggttgtttccaccttttgactatgtgaataacgccactatgaacatttgtgtacaggtttttgtgtgaaccagtcttcagtcctctttagtatatatttaggtatggaattgctgggtcatatggtaacttgaagtttaactttttgaggaactgctaaatccaccatctgattttgaactgaacaccaaagccccagtgggaatcacagagaatcttaactatgtctgtctaggttcaagaatgttagcttaaaagaaggcctaagagagcggaacaaaccctgattgtccaggaacccctcagtaactttacctttgctccctgccacaacacacatacacacagacacacacacacacaatctgatttacacaaaaatttgctcagtcaatcctgttttctctgccaagagcatccaccctgatctcaagggtattattccacaataagcagcccataggactactgaaaaaccaggaagttacatttatgccaaagctgttcagaggttgtgcctcagaagacacacagcaggaaccgtgtgggctctgatcaaagcaagaatgtcatcccagagttgctccagtattgcttcacaaatattacaaataaaacacgatgctttttgagtcgtcatggctcacacaaagggtaggtttcaacatcagcctctggagagccacattcccagagtggccgtcccggataagaaggccactacagcacgactcacagcaccatggaggtttcctccacctgcgggacacagcaggatggagacggttgggcggcctcccttcctgggacactccatggccagcccctcagggactacttgaagaacagaacccccaaaagaccagtttctgaaaagggactcaaagtctaagggaggcagcctccttttatccccaggggagggcagctctggggcagacaggttcatctggatgtcatattcactcccgcacagaaaacctaggccaagcaatttgtccatcctcctcagagccagttttgtggtccccatggtctgcccaccacctgtacctggacgctggcttcatttggccaccagacaaaagtcaggctggccctggagtccaccggcctgcccagcccatcacttctgggctaggtagtgctaagcctgcccagcccaccctggctggggctcagtgccctcttcttggtgacacactgtgctccttggagaactcctctctaggtgctggggtgagggcaggtgtgatctgagatcccggggctctctggcaggtgcatgcacgcacactggaacctgatacaggcttttgaacacaggtgcacagtccagaaacctgcaagtcccatcccaaactatatctgactctctgccctatgcctgtcctcctacaggatcatcagcccccaagagacctcaccactccccatcctcaccttaaattccagggtcaaaaagactctccttgtagggaaaggtagggtgtgttgatcaggtggtgttggaaggagagccccaatataggccactaattcagttccaggtggactgcacgcgtcccatcagagttagtttactcaccgctggtctttgtccctgcagcacccagggcttcgggaagggagaagcagacttcctagtgctgcaccccccagctcccccgagcctatgggcactgggttgactccagtttctttcctcttagagactgaggtttctccctttcctctcacacctgcttctgtccggaccggccgcttctacccagcggaccctcggacggtcctcgcaagatccgggttacacccgccccgtgttccctcttccccggttcgcccttcccggcctaagcccatcttccttaggaaaacttagcagggatttaggggccaaaccaggagggtggagttttctttgccctcggccaggagcacatctccctgttgcgttttttgggggagacagagaggggcgggcctgagtcgagtatccctctcctcgcagcgccacaactgcgcgcaccgacctccccacgccagacgcggcgtgagccaggcctctaatccgctcgcccctcttccgggtcccggtcccgcccggggcgcccggggtctcgagagcccgcctggcggcgcgatctcacctgtcggccgcgcccgcgcgctctcgctccggctcccgctcccgctcccgctcccgcttggtcgtcttcccgagttactgacccgaggcgctcccgggcggggcgtgactcagcactcgtgctggtccccgaccccgcggcaacgccccggccaggccccggaaacggtcctggcacgctggggctggtctccgaacggcccgccggactaaccttgaccctgagcgaacggccttgaactttgaaacaaatgctcccatggccagatgtcgcctctccggctcctgccagcacccgcgccccagtcctgccgagccattcagacttggtttaaagtgtcctttcagcatactttaaagcaaaaaagagagagagagagagagagagagagagagagagagagagagagagagagagagagagaaggaaggaaggaaaacaagcatccccctgttcctctgattttctcgaggacacctttaaagccactggaaccttggagagtggggttcccctcacacgttgggcgcactgccaacatccttccttcagacatacacataataattccgcaaaccctgttgagctcctacatttgccaggcactgtgctaggcgttggagggataacttggagagaaaactgacaaggttcctgctttcacggaggcgacagtctgggaaaagacagatattaaacacaccaccaaaaaatacatgtatagagaaaacaaaaaccatgtatcaccaactgcagtaagaaagattgggcatggccaggcacggtggctcatgcctacagtcccagcacttagggaggccaaggcgggaggatcgtttgagcccaggagttggagaccagcctgggcaacatagcgagacctacaaaaattagccgggcttgttggtgcacacctgtagtcccagcttctcgggaagctgaggtgggaggattgcttgagcccaggagtttgaggctacagtgaaccatgatcattccactgcactccagcctaggtgactgtgagattctgtctcaaaaaaaaaataataataataataataataaatgaaatgaaatgaaaaaataaaaaagaaagactgagggcaaaggaagggtgcaaatacttctctccaaggatgagaaagaggtagatttaggtaaagaggaaggaagtgtggtctggatgtgcaaagacccacaggcaggacaatgacctggaaggcagtgtattgccagctcaaatcagaccaagctaactcagcgaaagataagccatctccatctacttactgcaaatattagcacctactttaagagttgtggataagggccaggcacggtggctcacgtctgtaatccgagcactttgggaggccgaggtgggtggatcacctgaggtcaggagttcgagaccagcctggcaaacatggtaaaacactgtctctacaaaaaattagcctggcatggtggcacatgcctgtagtcccaactactcaggaggctgaggcaggagattcacttgaaccaggaaggcagaggttgcagtgagcggaggtcatgccattgcactccagcctgggcaacaagagcgaaactccatctctaaaaaaagaaaaaaagagttgtggaaaggcctcaatgagatcatttttataaagtgttttaacatggtgcctggcacacagtagcaatgaatacacagcagccatttattatagatattttatattgcagattattttgcccaaggcaaccagaatgatttatttttgatgaaaaatcctactcatcgtcttagaaaaacaataagctaatatttattgatcacgaactaagtgctttgaatgacttctcaagtttttttgtttgtttttgtttttttcacaaaggtcctgcaaggtaagttttattaatctccattttgcagataaggaaatttaggctcctagaggctaaagaatttaggcaaggtccctcagctagtaaagcaagggggtgggcctacaatggaggtctgaaccatgacccctttttggggggtagggggacgaatctggctctgtcgcttaggctggagtgcagtggcatgctctcggctcactgaagcctccaccttccaggctcaagcgattctcatgcctcagcctcccaagcagctggaattacaggcacctgccaccacacccggctatagtagaaacggggtttcaccatgttggccaggctagtctcaaactcctggcctcaagtgatctgcccacctcggcctcccaaagtgctgggattacaggtgtgagccactgagcccagcctgaaccatgaccttaaagccaggaaacccctcagcaagcatcatccctaaggagccagagggcactgctcaggcaaagcagaatctcagttgtggggaatttactgggcatgtacccagtaagatgtgacacagagaggtgaacagagaggctacagcctgcccactgcacggaatcagccctgaggcttgggatggtcatggaaggctttgtgtagaaggtaggcgtgcaaggatgaaggggatgccagctggcaggatgcaaggaaaggactccagaaagagcaggacaaagataaagctggaagagaggcgagagaggtgtgaggtggcccaacactgggctcaggtgtgccagggctggtgtggcccatctctaaggacctgtcccaaaccatgaaaaactttttttttttccttaagacctgctacaaagcaacaaaccatgaaaatcaaggaatttttctgattatcactcaatactttcttggcaaccagaagacacctccatccaggagaggggacatggtctcatgcaacccctccaggtagctcactgcacaccactcctctggtcccactcagcagtgcccatagctttggaactgaaaatcagggctcgctgcctaaaaaaggggttttgtctctgaaattccaagtgccagaggtagcaggaaagtgcggtggttaggatcctgaaaaagtggcaagttcattccatggaactttgacggttagtggggagagcaggcagattgtctaacatgacggaatctaagaggaggaagcgttgctggctctctgctgtaatgagctcagccaggcctgtgggtacttccttacagacaacaggtggcttttctgctggctcccagggctggaggagctccaaggtactgggtcatctcaggagcccagagaaacagaggcagtatgcccactcgaagaacaggcaactcttagatctgaagagcctggtaaatgttctccttttccatacgtagttccaacgaaatttgtaagcttcaaagaagagaggaatatcctcgtttcttcctctctcccccaacacatgatttggtatagtgttcccagctagggagaggctatattaatgaaggtaagttctgctgcaatgaccaatagattccaaatgtaatggctcaaatacaacagaagtgcacttcttgctagggcatgagttccaggccagtggactggtttatgaggctgtctcccttcacacaggcatccagggaccaggctaacaggacatggcaagtgagtgccactcatggtcccaaattagatctttgtttttcttttttagaaacagggtctccctctgtcacccaggctggagtgcagtggcatgatcatagctcactgcagcctcgaactcccatgctcaagccatcctcctgcctcagcctcccaagtagctgggaccacaggtgtgcaccaccatacctggctaatttgtgtgtgtgtgtgtgtgtgtgtgtgtgtgtgtgtgtgtgtttgaagaaacagggtcttgctatgttgcctaggctggtctcaaactcctgggttcaagcctcccaaagtgctgggattacatgcgtgagatacagcacctggtctcaaattagggagctcatcagttctacctcctgtgttgtcacttgatctcaccaccaccagtcctgcctcagccatcttctctctcaccaagccactgtaactggtccctgggcctgtcttgcacttttccaattttctctccacaaggaagctgtgccacacttccttgtcccagctcctgccactctcagcctggaacactctgtttccagtccacttggtaaactctgaactctcttttcatggttcagagcctgaatcaccttgaagccttacctaacccaccccttctgggcccccagacatactcttccctggacagagcttccctgcttattgcactaatgggctgacacatcagtctccccagagtagcctgtgggtgccttgatgcctgtattagtccgttttcacgctgctgataaagacatacctgagactgggatgaaaaagagaattaatggacttacagttccacatggctagggaggcctcccaatcatggcggaaggcaaggaggagcagatcacgtcttacatggatggcggcaagcaaagaaagagagggctggtgcagggaaactcccacttttaaaaccatcagatcagtgagacttattacggtcacaagaacagcacaggaaagacccagccccacgattcaattacctcccaacaagttcctcccatgacacgtgggaattgtgggagttacaattcaagataaaatttgggtggggacacacccaaaccatatcagtacccacacgtgcaagaataaaccctcctccccgcccaccgcaactccctctcacagcaaagtcagacctcagatctaggattctgctaccattaacttactgtagaacttctcttaaactagccatgacctctggacccattcctaatctgtaaagggaggaacaaccatcccaggagtgttctgtgattgccccgtgttttaggaaaaactctcctcaaatcgtgttttccctctactctcatgccatcacaatcatcaacacagaagacttcagtgaccaaatgtgtgtggggtttccccaccaaaagcagtggacatcagccgggtgtcctctaattcagttccaacactatctacccagagacattgtcagaccccacaggttgagggttcagtctccaagactgcccccctcaaccac

SEQ ID NO: 99 - DNA, 4001 bp, Homo sapiens
  source 1..4001: mol_type = genomic DNA
gccagaacatttaacaggtaatgaatgcagtagcactccaaaattgaacttaaatgtaatgagccagtgacaacatttaacttagtatttggcactatgacaaaacaaatctcaatcaaaacttggacctattaatacttccaagttaacttttgattaaacataaattatacggtggcaggttggtaatgtcaaaacaggaaggttaagagataacacatcaaaccacttatccaattgaacacaaatgcatacagagatgttactgctgctaggtatttattggctcccactagtcccttctaaagagagaaatcaatggcagcctccaaatgtgaatggttactgaggaagttcatccacagattaagaacaaatattatgtgtagtcatacaaacaagaaaaaaatgttgggagggtgggacatttaacgtctctattaagtatatcaaactatgataacccctgagacctaaaatattctctcattccaaattccaaaaattctgcaaatgtgcataatggtttagtaatcaaatcttgtccttctctttattctaaaggatgggcaagtaaacactgtcatcactactttaacgtggaaacactcgagcacttgatgaaactgactaccaacagcaagaaaagtacaagaaagcagtttgaaatcgaatcatccaaataaatatcatattatctcaattcaaggttaatgagaccgggggagggggtatgggaggagtaaaaagggagctgcttcatccttgactgaagataggttttgcccttttccattgcctgtttatcaggaggtctaataatttcagatcctggtatttcgaggtttcttattttaagggcgaggtggaaaaggggtgtaaaataaaaccaatactcactgctaactttcatgctgccaaaagctgaaggctgctgcaccggcttgcagctctccgcaaaggaggtggttctgggccgccctgacatgatcactctcttcgcgaatcaccttttccttccttcctccttttcttccttttgtctttatgttggggtgttaggttaacgataaatgcagcattaagttctcccacagaagaaaaagaaaaagacttcgtcctcttggcttttcactccttttgtatactataaaaaacaaaacaagcgatatatttctccttcaagacagatcggcacggatatttaacatatagatgatttaggacttgggaaaaaatacaattctttcccctccctttcctgggaggagagaaaagagagggcaaatcctaaaaaaatatgtatcacgtgaaacgggggcaaatacttgattgaaaatgagtacttcgaatattatattttcctcggggattttttttccgagtcaatgaagaagggaagcggcggaaggatcacgagtctcacgcttgaagagaaagggggatgggtaggagggagggagagggagggagggggtggctcggagatgcgacgggaaacgctgcagctccggcaagccgcgggatccggcgggctgacggcaggggcccggcgaactagagggcggcggagtcgcgagtcagtcagaggcgggcggtggcggtggcggcggctcctctcctcattgcgccaggagcagctgcaggcggcggctggatccagcggccatggcggtggcggaggcagctcccttcagaccccaggcagcggctcctcgactgttccccattcgccggggacatgggaacccggcaaccgcttccgtccctcggggccccctcccccgtccgcggcaacagctcggccgccctcccgcccctcggctctgctcggtgcccgctcaggaagtgtccgcgctttgccccagcccagagccctgtcagcggctgcggggccggctcctcctcgcttccttccttcctttgtcacttggcccgggcggcggcggcggcggcggcggcacaagcccgcattcgcccgggtcaggagctgctctgtgtgaggagcgctgtctgcgcagccgccttgcacttccccactcctcctcccccctcctactcgtcctccacctccttcctcccccacccagccttacaccgcccagcgccccgccgcccgcgtaacaggcgtctgggaatcgccggccgagccccacgctcgcccgcttcggctgggagccgaagcccgaacggagccaatcacggacgtcgtttgtttgcggctcctcccggaaaaagccgatcagcctgagaaaccaattagaaagtgacgctgggaagccacgcccaaagagcggaaaatgccgaaggtagccgaacggaaagggtgaattcgccttgttcggtggctggctggaggagacattcgacaatggcaacagtggccaatcagagtcgccggcccttacgaagaggcttgaagagttgaggacggtgaaacccagagacgctggtgaaactgcacggctgggcgccgccgccgctttgtgattggctggaaaactccgctgggagcgtacctccttcggtgattggtgaagagaagggggcttccgaggacagtctggccagtcaggacttcccctccctctccaaggtggggagaagaaagcgaagggagggcgtctataaagcgctgtctcattggcctgaagcttaggcgctggagttggggcaaaagggagagggagggagcccgaaactcgcggcggcggccaatcggctctcactgccgtccaatcgcaagacctagctgcagtgtgattggccttcggccccggtcccaggagcggggagatgcctgactcgggtcgtggctgacgacccgacggttcgcctgcggccgcctccaacaacggtggaaaccgcacacagggaccgcgggccctcggtgcagcaaatcggaaagtgctgcggtcggcggccctgggctctgagcgctcaagcgagcagaggggtggaggggctctccccacccacaggaggctctcggcggggcgcagccaattctgcgcgcccaaggctcccctctgccgtggatcgcgtgtgccgagtgttggcctcttccccgaaagggccagacggaccaagggggccttcccagccggctgcaagctctcagacgctaaaatgggaacaaagcttccagaaaggcagcgcctttgggcggccgcgccccgcccagccgcgggggccattgatacagaggcttgctgcgcttggtgtgcgcggcccggccgggcctctggaaacgcccatgagaaatgagctcatgctggatgcgcgcgctccttgggcgcaactgcccagcgcaaaacgggccccgaaagcaggtgaggggaaatgccgtcaggccggatatcctgaagcctccgaggaaatgggggggtcagaggagcccgtggtagctttggatcttttggaactagggtgatctataaaagtgcagcttgagaagatttcatacataagaccagcgtccattgttctaccatagaaccagttcctttggtctgctttagaacagtgaggtatggctacgtcaagataacagagaaagtagaacagacagttgccatcctgattgtaatccagtggtgcctgtaatttctacctgcagagtcatctctttctgttaagatgctgtctccagaaagcacatgttaaaggacctatatttgatcagattacctggctcttgctttgaaacactgatgaaggacttgggacagaacaaagtaagctttgatggaagacaggaaaggtctagatagtgtttgcagattcaaatggctacagaggccagacaagtaaagcaaatgagtgagactggccttgtgtaagacaatggggaatgatagggaaaatggcaaattggaatatccatgccataactaaaaggaacagctgctcaactctagcagattgttgccctcaggaatgtgggtccagtgttctcagatgttcaatttttttcctcccaaaagaaaccccaaaattgg

SEQ ID NO: 100 - DNA, 4001 bp, Homo sapiens
  source 1..4001: mol_type = genomic DNA
caaatatccctattatttctaggaccatagccatctgcaaaagccaaaatggaaaggactaaatccacattcttctaactaaatccacagttatctaaatatgatggaaactaacgctagagtgttttaaatcgtcctttttaaggaacccgccagaacatttaacaggtaatgaatgcagtagcactccaaaattgaacttaaatgtaatgagccagtgacaacatttaacttagtatttggcactatgacaaaacaaatctcaatcaaaacttggacctattaatacttccaagttaacttttgattaaacataaattatacggtggcaggttggtaatgtcaaaacaggaaggttaagagataacacatcaaaccacttatccaattgaacacaaatgcatacagagatgttactgctgctaggtatttattggctcccactagtcccttctaaagagagaaatcaatggcagcctccaaatgtgaatggttactgaggaagttcatccacagattaagaacaaatattatgtgtagtcatacaaacaagaaaaaaatgttgggagggtgggacatttaacgtctctattaagtatatcaaactatgataacccctgagacctaaaatattctctcattccaaattccaaaaattctgcaaatgtgcataatggtttagtaatcaaatcttgtccttctctttattctaaaggatgggcaagtaaacactgtcatcactactttaacgtggaaacactcgagcacttgatgaaactgactaccaacagcaagaaaagtacaagaaagcagtttgaaatcgaatcatccaaataaatatcatattatctcaattcaaggttaatgagaccgggggagggggtatgggaggagtaaaaagggagctgcttcatccttgactgaagataggttttgcccttttccattgcctgtttatcaggaggtctaataatttcagatcctggtatttcgaggtttcttattttaagggcgaggtggaaaaggggtgtaaaataaaaccaatactcactgctaactttcatgctgccaaaagctgaaggctgctgcaccggcttgcagctctccgcaaaggaggtggttctgggccgccctgacatgatcactctcttcgcgaatcaccttttccttccttcctccttttcttccttttgtctttatgttggggtgttaggttaacgataaatgcagcattaagttctcccacagaagaaaaagaaaaagacttcgtcctcttggcttttcactccttttgtatactataaaaaacaaaacaagcgatatatttctccttcaagacagatcggcacggatatttaacatatagatgatttaggacttgggaaaaaatacaattctttcccctccctttcctgggaggagagaaaagagagggcaaatcctaaaaaaatatgtatcacgtgaaacgggggcaaatacttgattgaaaatgagtacttcgaatattatattttcctcggggattttttttccgagtcaatgaagaagggaagcggcggaaggatcacgagtctcacgcttgaagagaaagggggatgggtaggagggagggagagggagggagggggtggctcggagatgcgacgggaaacgctgcagctccggcaagccgcgggatccggcgggctgacggcaggggcccggcgaactagagggcggcggagtcgcgagtcagtcagaggcgggcggtggcggtggcggcggctcctctcctcattgcgccaggagcagctgcaggcggcggctggatccagcggccatggcggtggcggaggcagctcccttcagaccccaggcagcggctcctcgactgttccccattcgccggggacatgggaacccggcaaccgcttccgtccctcggggccccctcccccgtccgcggcaacagctcggccgccctcccgcccctcggctctgctcggtgcccgctcaggaagtgtccgcgctttgccccagcccagagccctgtcagcggctgcggggccggctcctcctcgcttccttccttcctttgtcacttggcccgggcggcggcggcggcggcggcggcacaagcccgcattcgcccgggtcaggagctgctctgtgtgaggagcgctgtctgcgcagccgccttgcacttccccactcctcctcccccctcctactcgtcctccacctccttcctcccccacccagccttacaccgcccagcgccccgccgcccgcgtaacaggcgtctgggaatcgccggccgagccccacgctcgcccgcttcggctgggagccgaagcccgaacggagccaatcacggacgtcgtttgtttgcggctcctcccggaaaaagccgatcagcctgagaaaccaattagaaagtgacgctgggaagccacgcccaaagagcggaaaatgccgaaggtagccgaacggaaagggtgaattcgccttgttcggtggctggctggaggagacattcgacaatggcaacagtggccaatcagagtcgccggcccttacgaagaggcttgaagagttgaggacggtgaaacccagagacgctggtgaaactgcacggctgggcgccgccgccgctttgtgattggctggaaaactccgctgggagcgtacctccttcggtgattggtgaagagaagggggcttccgaggacagtctggccagtcaggacttcccctccctctccaaggtggggagaagaaagcgaagggagggcgtctataaagcgctgtctcattggcctgaagcttaggcgctggagttggggcaaaagggagagggagggagcccgaaactcgcggcggcggccaatcggctctcactgccgtccaatcgcaagacctagctgcagtgtgattggccttcggccccggtcccaggagcggggagatgcctgactcgggtcgtggctgacgacccgacggttcgcctgcggccgcctccaacaacggtggaaaccgcacacagggaccgcgggccctcggtgcagcaaatcggaaagtgctgcggtcggcggccctgggctctgagcgctcaagcgagcagaggggtggaggggctctccccacccacaggaggctctcggcggggcgcagccaattctgcgcgcccaaggctcccctctgccgtggatcgcgtgtgccgagtgttggcctcttccccgaaagggccagacggaccaagggggccttcccagccggctgcaagctctcagacgctaaaatgggaacaaagcttccagaaaggcagcgcctttgggcggccgcgccccgcccagccgcgggggccattgatacagaggcttgctgcgcttggtgtgcgcggcccggccgggcctctggaaacgcccatgagaaatgagctcatgctggatgcgcgcgctccttgggcgcaactgcccagcgcaaaacgggccccgaaagcaggtgaggggaaatgccgtcaggccggatatcctgaagcctccgaggaaatgggggggtcagaggagcccgtggtagctttggatcttttggaactagggtgatctataaaagtgcagcttgagaagatttcatacataagaccagcgtccattgttctaccatagaaccagttcctttggtctgctttagaacagtgaggtatggctacgtcaagataacagagaaagtagaacagacagttgccatcctgattgtaatccagtggtgcctgtaatttctacctgcagagtcatctctttctgttaagatgctgtctccagaaagcacatgttaaaggacctatatttgatcagattacctggctcttgctttgaaacactgatgaaggacttgggacagaacaaagtaagctttgatggaagacaggaaaggtctagatagtgtttgcagattcaaatggctacagaggccagacaagtaaagcaaatgagtgagactggccttgtgtaagacaatgg

SEQ ID NO: 101 - DNA, 4001 bp, Homo sapiens
  source 1..4001: mol_type = genomic DNA
accccggaattttaaaatatttttcaaatcactccatgaagtgcttagaaagcacgacccttccctgaaagatgcataatcagctttcctctttacctacccgggcattctctacttgcaagccacaagaaaaaaaaaaaattaactttcaaaaattatctcccattcagctattacctagactaccctgtttcaggctagttcccaaagccaacatgaattgaaatcttgagttcaactaaaagtgatcattttatgttgcctaagaatataatcaatttcacataacatcatgttttgcgtgtgtgtgggttcttaaaatctcagttctttagttctctcagttcttctaggcaacaaattaatttgtctttgccctcatgatgcccctctttcccacaataaggaaacaatatagatatttttgttaaattctgcaagtatgtttaaacaagctatcattaaataccacatgaacttctccataaatagagatacactggaacattagatacttagtatatattaaacacttcaagagcatacttttcagcacaaacccaggacaagagtagtctatccacaaccctgaacacagcccaaatatccctattatttctaggaccatagccatctgcaaaagccaaaatggaaaggactaaatccacattcttctaactaaatccacagttatctaaatatgatggaaactaacgctagagtgttttaaatcgtcctttttaaggaacccgccagaacatttaacaggtaatgaatgcagtagcactccaaaattgaacttaaatgtaatgagccagtgacaacatttaacttagtatttggcactatgacaaaacaaatctcaatcaaaacttggacctattaatacttccaagttaacttttgattaaacataaattatacggtggcaggttggtaatgtcaaaacaggaaggttaagagataacacatcaaaccacttatccaattgaacacaaatgcatacagagatgttactgctgctaggtatttattggctcccactagtcccttctaaagagagaaatcaatggcagcctccaaatgtgaatggttactgaggaagttcatccacagattaagaacaaatattatgtgtagtcatacaaacaagaaaaaaatgttgggagggtgggacatttaacgtctctattaagtatatcaaactatgataacccctgagacctaaaatattctctcattccaaattccaaaaattctgcaaatgtgcataatggtttagtaatcaaatcttgtccttctctttattctaaaggatgggcaagtaaacactgtcatcactactttaacgtggaaacactcgagcacttgatgaaactgactaccaacagcaagaaaagtacaagaaagcagtttgaaatcgaatcatccaaataaatatcatattatctcaattcaaggttaatgagaccgggggagggggtatgggaggagtaaaaagggagctgcttcatccttgactgaagataggttttgcccttttccattgcctgtttatcaggaggtctaataatttcagatcctggtatttcgaggtttcttattttaagggcgaggtggaaaaggggtgtaaaataaaaccaatactcactgctaactttcatgctgccaaaagctgaaggctgctgcaccggcttgcagctctccgcaaaggaggtggttctgggccgccctgacatgatcactctcttcgcgaatcaccttttccttccttcctccttttcttccttttgtctttatgttggggtgttaggttaacgataaatgcagcattaagttctcccacagaagaaaaagaaaaagacttcgtcctcttggcttttcactccttttgtatactataaaaaacaaaacaagcgatatatttctccttcaagacagatcggcacggatatttaacatatagatgatttaggacttgggaaaaaatacaattctttcccctccctttcctgggaggagagaaaagagagggcaaatcctaaaaaaatatgtatcacgtgaaacgggggcaaatacttgattgaaaatgagtacttcgaatattatattttcctcggggattttttttccgagtcaatgaagaagggaagcggcggaaggatcacgagtctcacgcttgaagagaaagggggatgggtaggagggagggagagggagggagggggtggctcggagatgcgacgggaaacgctgcagctccggcaagccgcgggatccggcgggctgacggcaggggcccggcgaactagagggcggcggagtcgcgagtcagtcagaggcgggcggtggcggtggcggcggctcctctcctcattgcgccaggagcagctgcaggcggcggctggatccagcggccatggcggtggcggaggcagctcccttcagaccccaggcagcggctcctcgactgttccccattcgccggggacatgggaacccggcaaccgcttccgtccctcggggccccctcccccgtccgcggcaacagctcggccgccctcccgcccctcggctctgctcggtgcccgctcaggaagtgtccgcgctttgccccagcccagagccctgtcagcggctgcggggccggctcctcctcgcttccttccttcctttgtcacttggcccgggcggcggcggcggcggcggcggcacaagcccgcattcgcccgggtcaggagctgctctgtgtgaggagcgctgtctgcgcagccgccttgcacttccccactcctcctcccccctcctactcgtcctccacctccttcctcccccacccagccttacaccgcccagcgccccgccgcccgcgtaacaggcgtctgggaatcgccggccgagccccacgctcgcccgcttcggctgggagccgaagcccgaacggagccaatcacggacgtcgtttgtttgcggctcctcccggaaaaagccgatcagcctgagaaaccaattagaaagtgacgctgggaagccacgcccaaagagcggaaaatgccgaaggtagccgaacggaaagggtgaattcgccttgttcggtggctggctggaggagacattcgacaatggcaacagtggccaatcagagtcgccggcccttacgaagaggcttgaagagttgaggacggtgaaacccagagacgctggtgaaactgcacggctgggcgccgccgccgctttgtgattggctggaaaactccgctgggagcgtacctccttcggtgattggtgaagagaagggggcttccgaggacagtctggccagtcaggacttcccctccctctccaaggtggggagaagaaagcgaagggagggcgtctataaagcgctgtctcattggcctgaagcttaggcgctggagttggggcaaaagggagagggagggagcccgaaactcgcggcggcggccaatcggctctcactgccgtccaatcgcaagacctagctgcagtgtgattggccttcggccccggtcccaggagcggggagatgcctgactcgggtcgtggctgacgacccgacggttcgcctgcggccgcctccaacaacggtggaaaccgcacacagggaccgcgggccctcggtgcagcaaatcggaaagtgctgcggtcggcggccctgggctctgagcgctcaagcgagcagaggggtggaggggctctccccacccacaggaggctctcggcggggcgcagccaattctgcgcgcccaaggctcccctctgccgtggatcgcgtgtgccgagtgttggcctcttccccgaaagggccagacggaccaagggggccttcccagccggctgcaagctctcagacgctaaaatgggaacaaagcttccagaaaggcagcgcctttgggcggccgcgccccgcccagccgcgggggccattgatacagaggcttgctgcgcttggtgtgcgcggcccggccgggcctctggaa

SEQ ID NO: 102 - DNA, 4001 bp, Homo sapiens
  source 1..4001: mol_type = genomic DNA
gtgatgctctcagtgcgtgatcggcgggtcagccccacctggctgggcgggggattgttgagatggtgccggcgcagggggacgctgatggggttagagcagttggacgaggactggctcttgctgcgagggcggaactcatcactcatggcccgcatggcctccaggatggtctcgtgcatgttctgggccaccacagagtcatccacctgcatccagaactccccgggccccgtcacggcagaacggcccacctcgatgaagaagaagttttccgagtggccacagcgcctgatgttcatcagctgcagcaccacggccgctgcctccgagttcagcttcacgaagctgatggtcttgctggtcaggcaaaggcggtagataccaatcaggttctttgtctgacccaggcccttgggcttcaggatcacttgccagacctctttgaatgcgggtcctgggggcacgtcaccgtagctcaagtcctccccagcctcaccaaggccggagctgccgctgcagctgcccccaccacctcccgccccgagggccgcagctccgtcgtggtggcccttagcacggttgtgcagctgtaggagagcctggtaccagctgtcttgctcggcctcgctgtccgccgcgatggcaaagtgctcgtcccgggtgtagagagccaccaggtgcttgttcttggagtcagcccgcttgttgatgttgaagcagctctcaagggggatcgagcgtttgggggcgctcgacttgtgccgccacttcttctcgttctcgtagtactcgaggcgcgccgggcccccagcctcgctggccgcgcgcagtacgaagaagcgtttgtgcatgctcttgggtttgcgcaggtagcccaccttgcgcacgtccgagaagccatcgctctccggagggctcgccatgctgccaccgccaccaccaacgctgagcagagggaggctccgaaaaacaaccgggtggggggcggaggctcctcgccgcggcccggcacatgcaaacagggctggaggcagcagaaaccccgactctgaaatccacgccgccccccgcgccggggaggggcagctgaaggaggacgcagctgctgagcccaggagagagcccgaccggagttttcgggcgcttcacgcccggcggggaggcagtgcgtccggggtgagggcagccccgatcctccgagagccaagtctcctctcagccgccgccgccaccaggaaggagcgaagatgcatctctcgtcgccccggctggagtccggcacagggaggcgacagtcgggggtccctgcggtgcccctccaggagcgcgcgcgcgcgcgcgccttccctcctgagttcccctctggaagcagcgattcccgaggcaaattaaatatccttgggcagggggaggcgggttgccaagtcccaacgttgcacggggttctccctcctccttcgcctcctcccaccccccaaccgtcccagccgccaccagccgcccaaataccagctcagtcgcagagaccgcggcgctgcggctgttgctgttgctgctgctgctgctgctgctgctgccgccgcccgcgggcgcgtcctctgcagcccccatccgggcccatctcggcgggtggagaaagtggcttttccatgcgaaacgataccgggcagccactgcagctggggaccggccggagggacagactcatccctgcccctcgctccaggcggctggggaggaggggaggggacaagggcgagaggggatgggggaggtttgggaagggttcggggaagacgcctgttcctcgggaggcgctgccgctgcagttacttctcccctcctccctcctcctcctcctcctcggagagttgccgagagccccaaccaaaacaagcggcggcgtctggctctgcgcgccggccccctccgaccgcgccgccgtctcactcggaggagaaaaacacgtgacggagcctccgcgctcggcagccgggcagccgccgccgggaggctcccgcccgggtctctacattccgggccgagcccgccccgcgcccgcccctctccgccccccagccgtgctgcagcggcgcccgcgccgaccccggcgcagcgctaggacagcgcccctgcgcggccgccagctccggcgcagcgcctccctacacgcgcgccaaccggggcgcccccggcccgcaggaatccccgcggcgggcgcaggcgggggtgtttgggtggctacccgaaagcacgaggggagtctgggaaaacgggagggggcgagctcgggaagacgcaggagtgagggtggggggccacgggggggcactgcggctacagagccccgagtgggaggccggggggaggcgggggcgccggagctgggctgcctcttacctatcaggtaagactgacccacgggagggaggtttctagtcaaggctgtttcccccaggaaatttaaaagctagatttcggcttcagggcgataggccatggtgaggggatctgtattttttaaggtttttctaaaggacttctgggccagccttccaccttgggatccccagatttgctgccgtgaggtacctggaaggaacagagggacgcccaagggcagtgactgggggcagttatggtcactacaaaaggagaacagggatccccaggaagtgtctcacgtccggcgctgtggaattagttgctgatcaagtagggagaaaatgtaaatatcaccttttctcctgctccccttgcccatccgtgtgacggggggactcacacctaaggctgagggcatgggggaggggaaatgagaggtcccttatcatattccgaagtctatccgtgactttaaaaagctgttagggaccttcagagggttcataaggccaaaaccctactgtgcaaatattggctgaaatgtttgtttcacacacagctcccacacccattgaaccaccctataagtttttaaccttgaccacaaggataacagggagaaaacaaggatggtgccagctcaactgtacaggaaaatggttacttcatgagtggaccagatcagcagcctgggtctacttgaagcgcccctttcagggtcaccttgcggtatcagtctccccacaaacaaacgctccccagagtttttttattttttaagccgacctataaacaagcagtttgtccctgcttgctcgcctggcactaaggacccacagagatgtcactcggtttaatgctgtctcattttgatgcctggcccgtttggggttgccgcccctttactacgccgggaccctcctcggcctcaggctcccaggcactgcaggatgctggctgggcactcagcagtcgaggcgctgggcatgctcagtggctgggacccggcgggccgcgggggagcgcgcctcggcccaatgcggctgaggggcggagcgagtttcccggggaggcctgctgattggtgggatggcctgtttttcaagccttgcactgcagagggaaggtgggggtagaagggttgagggtaccaaggtaggttgcaaagcgtagtaaaaaaagaaaaagaagatgatgatgatgaaggagaaaaagaaccataggcgccattcaggggtgtgcgatacgtaaattagggggattgggcatctgcagaggggctcctccccacccaagcctgggagcctctcgtttctcctctccccttcctgttcccggtcgtcatgcagcctcatctaaacctggttaatctataatttgagtacacatgttgaattgaaagccctcccttcttttcctctgacagcttggtgatgtgggcacacgtgcccactacagcaaggctattaatagcagatatttggaaatggaagcgaggcggcactcacagtgaccgaaattctgggggtatagagtgccttacagggggtagggcagggaggtgcagaataaagaagcctgaagttggtggaaaagtgtaactgtgtgcctgg

SEQ ID NO: 103 - DNA, 4001 bp, Homo sapiens
  source 1..4001: mol_type = genomic DNA
ccgattatccagatctgcagcactggctgcttcatccccagccaaggctggactcgtgcccaagcctgttcctggggtgcagcggtggccattgccaccccgagacaaaatgtagtgaccgttgggggcggtcagggtggagggccccttgccacccatgcagatatagttgcttagctcctcctcaccgcgggctggtggggtgtggcccagggaatccggagtgacactgcggaaggaactccggaaatcgcagggactggagccatactcatccgaggagatgaaaccgccatcgctgggggaaccagacaccgaagcactagatcgccgtgggaagagacaatccgaggtggagccatggccactggtgctactggacgacagactgaccgggctggtggccgaaggcgagcagcgggaagccggcatggggatggagcggctgtggttgagcggggggtgcagccgggcgctgccccgatgccggtgggcgtgggttctgttggtgctgggactcacagggctgccgtccaccgaggctgggcgggacatggtgccttcgccgtcactggaggcgcggacacggaaggagcctggcttcccgcccaccatgctggccggggaggtggcggtgatgctctcagtgcgtgatcggcgggtcagccccacctggctgggcgggggattgttgagatggtgccggcgcagggggacgctgatggggttagagcagttggacgaggactggctcttgctgcgagggcggaactcatcactcatggcccgcatggcctccaggatggtctcgtgcatgttctgggccaccacagagtcatccacctgcatccagaactccccgggccccgtcacggcagaacggcccacctcgatgaagaagaagttttccgagtggccacagcgcctgatgttcatcagctgcagcaccacggccgctgcctccgagttcagcttcacgaagctgatggtcttgctggtcaggcaaaggcggtagataccaatcaggttctttgtctgacccaggcccttgggcttcaggatcacttgccagacctctttgaatgcgggtcctgggggcacgtcaccgtagctcaagtcctccccagcctcaccaaggccggagctgccgctgcagctgcccccaccacctcccgccccgagggccgcagctccgtcgtggtggcccttagcacggttgtgcagctgtaggagagcctggtaccagctgtcttgctcggcctcgctgtccgccgcgatggcaaagtgctcgtcccgggtgtagagagccaccaggtgcttgttcttggagtcagcccgcttgttgatgttgaagcagctctcaagggggatcgagcgtttgggggcgctcgacttgtgccgccacttcttctcgttctcgtagtactcgaggcgcgccgggcccccagcctcgctggccgcgcgcagtacgaagaagcgtttgtgcatgctcttgggtttgcgcaggtagcccaccttgcgcacgtccgagaagccatcgctctccggagggctcgccatgctgccaccgccaccaccaacgctgagcagagggaggctccgaaaaacaaccgggtggggggcggaggctcctcgccgcggcccggcacatgcaaacagggctggaggcagcagaaaccccgactctgaaatccacgccgccccccgcgccggggaggggcagctgaaggaggacgcagctgctgagcccaggagagagcccgaccggagttttcgggcgcttcacgcccggcggggaggcagtgcgtccggggtgagggcagccccgatcctccgagagccaagtctcctctcagccgccgccgccaccaggaaggagcgaagatgcatctctcgtcgccccggctggagtccggcacagggaggcgacagtcgggggtccctgcggtgcccctccaggagcgcgcgcgcgcgcgcgccttccctcctgagttcccctctggaagcagcgattcccgaggcaaattaaatatccttgggcagggggaggcgggttgccaagtcccaacgttgcacggggttctccctcctccttcgcctcctcccaccccccaaccgtcccagccgccaccagccgcccaaataccagctcagtcgcagagaccgcggcgctgcggctgttgctgttgctgctgctgctgctgctgctgctgccgccgcccgcgggcgcgtcctctgcagcccccatccgggcccatctcggcgggtggagaaagtggcttttccatgcgaaacgataccgggcagccactgcagctggggaccggccggagggacagactcatccctgcccctcgctccaggcggctggggaggaggggaggggacaagggcgagaggggatgggggaggtttgggaagggttcggggaagacgcctgttcctcgggaggcgctgccgctgcagttacttctcccctcctccctcctcctcctcctcctcggagagttgccgagagccccaaccaaaacaagcggcggcgtctggctctgcgcgccggccccctccgaccgcgccgccgtctcactcggaggagaaaaacacgtgacggagcctccgcgctcggcagccgggcagccgccgccgggaggctcccgcccgggtctctacattccgggccgagcccgccccgcgcccgcccctctccgccccccagccgtgctgcagcggcgcccgcgccgaccccggcgcagcgctaggacagcgcccctgcgcggccgccagctccggcgcagcgcctccctacacgcgcgccaaccggggcgcccccggcccgcaggaatccccgcggcgggcgcaggcgggggtgtttgggtggctacccgaaagcacgaggggagtctgggaaaacgggagggggcgagctcgggaagacgcaggagtgagggtggggggccacgggggggcactgcggctacagagccccgagtgggaggccggggggaggcgggggcgccggagctgggctgcctcttacctatcaggtaagactgacccacgggagggaggtttctagtcaaggctgtttcccccaggaaatttaaaagctagatttcggcttcagggcgataggccatggtgaggggatctgtattttttaaggtttttctaaaggacttctgggccagccttccaccttgggatccccagatttgctgccgtgaggtacctggaaggaacagagggacgcccaagggcagtgactgggggcagttatggtcactacaaaaggagaacagggatccccaggaagtgtctcacgtccggcgctgtggaattagttgctgatcaagtagggagaaaatgtaaatatcaccttttctcctgctccccttgcccatccgtgtgacggggggactcacacctaaggctgagggcatgggggaggggaaatgagaggtcccttatcatattccgaagtctatccgtgactttaaaaagctgttagggaccttcagagggttcataaggccaaaaccctactgtgcaaatattggctgaaatgtttgtttcacacacagctcccacacccattgaaccaccctataagtttttaaccttgaccacaaggataacagggagaaaacaaggatggtgccagctcaactgtacaggaaaatggttacttcatgagtggaccagatcagcagcctgggtctacttgaagcgcccctttcagggtcaccttgcggtatcagtctccccacaaacaaacgctccccagagtttttttattttttaagccgacctataaacaagcagtttgtccctgcttgctcgcctggcactaaggacccacagagatgtcactcggtttaatgctgtctcattttgatgcctggcccgtttggggttgccgcccctttactacgccgggaccctcctcggcctcaggctcccaggcactgcaggatgctggctgggcactcagcagtcgaggc

SEQ ID NO: 104 - DNA, 4001 bp, Homo sapiens
  source 1..4001: mol_type = genomic DNA
gagttcaggagttcgagaccagcctgaccaacatggagaaaccccatctctactaaaaatacaaaattagccgggtgtggtgggcgcctctaatcccagctactctggaggctgaggcaggagaatcacttgaacctgggaggcagaggttgcagtgagccaagaatgtgccattttactccagcctgggcaacaagagcaaaactagtctcaaaaaaaaagaaaagaaaaaaaaagaaatctcaattcccacgtcctccacaggtcatgtagccatccccctgcctcctggagagagggtaactccaggagccacacctccttttcaggtaggaggtgcctaggccagcctccctcaccagcaccccaggcccacagcaggcaagcacccctctcaggagggccaccctcctccccacggccccttcaaggcctccgtgcccgacctttgtccatgttgctccatgcctgactctcgctgaggttatcttatctcccagatgaactgagcaccctgagggccacgacagtgcctgccctcccccaaccccctccccatggtgccccgtgaaggaaagctctgttcttgctcactgagagagcaaatcattgccaactacatgtccaaaatcagcccacttctgccttgataacctctaggccctcagctgctctctaaaacaaagccctgagggcccctgggccagggtaaaggccataaaggggggatggagggtggcaaggggattgctcaaggccagcttggtggaggaagccaatgcccaggctggccaagtccttcccatcggccatggttctctccctccacccctctctgtcagtcatccagctgagcagggcacacgcctgcaggtgtgtccaccacccccataccccactccccacctggaggatggaagtcccaacacagaacccaactctctgtccctacaacagacgctggctctggcaacatcggcagggctccaggccagcctaaccactggagcaatgaatgggccacgcagccactctcagaagggtctgcaaggacaagaacgcctctaacactgccccttgtccggggcagctgctgattcccctgtcctcggacaggcctgggctcctctggccccctccctggcaggccagccctcctctgggagcctttcctacccgcaaggcaccgctcatagcaccaagaaggaacctatcaccacaaactgcccagatcttatctgactccttccatataaggggacactgtccagggccgggcccccaccagcccgcccctcgaaaagctcctcaggagcccctgaggatgcacagacccaagaggatctggatgcgggacacctgccggctcccctctgcctggccaaggccagctccagagcccctacccagtgaccttgtcaggtgactctccccaagcctcagtttccccacagggcccatagggcacaccgatccccgcttagcccagggcccatgttctcggactcggagctcgcgcttcggccaaccccggaaagtcgcggggcccttgggtcagaggttagggggtcaggcgctccgctacctcagctcccagtggccgctgcccacgccggcgtctccgtccttgttgggggccggggctggcgcgtcccccgcggctcccacgtcggggcccgcagcggcgtcccgcacctcctcttccgccgccgcaggcccgccgccgccgtggctcgagggccgcgaccctgtcctccggcgccgggagctgccgcggtcgcccatggcctcagcccgcacccggccgcagccaagcgtgggcccgccgggttcgtagcgcccgaggcgcgcggccccacctccgggccctagacaacggccgccactgccccctgccggccgccgtagcccgggtgaccgcctcaccagcgcgttcaacccgcccgcgtcgggcccgtcggcctcaaggacaacggctgcgttgctccggagccgctaactaatggacggccgcctctctcgtccatttgtagtccgcggccgacctgaggcggggcctggagaggctgacggggcgcagggcaccctgggagtcgtagtcgccgcgggcgcgccggcgcagagcaccctgggagtcgtagtcgccgcggccgcaccggcgcagagcaccctgggagccgtggcagctcccagggccggggctgcgggccgcaagtgcgcgcagcgggaacggaccttgcatcgctcccgcgtggtcccggggtcctgtccggccccacggagcgggatggtccgctgaggggtgcgctgggccccgacttcctggccgctgtgcgcccgcccctcgtcccacttgtgtacacacggggtctcggcgagtcactccgggcaatcatttaggaagcgcctagtgcgccacggtccagctgtccgggccggtgtccgcggcggctcgcgtcgcccgccacccagcgcgttggtttctgcgcacgcagcttacagtaacgtccctggcgcgcttctgctccgcgagccctctggttaccccggcaggaacctgcttttcttcgccttagggggacgtcagacaagggccaggtcagaagaccaagctggagaaatgcaccaggccctgcgtcaccgggacgcctgccttgcacgcgactcccaggctctccacctccccgcctgcagagcccggcgagaggagggcgagggcgagggcgagggcgcaggccccttggtggtgtcttctcagcccacccactctctccacaaggagctccacgtccctcaactctcagggcctcccagtttccaacgtgggccagcctctccaggatgtcagaagtctgggccgctggatcgccgggactcaagaggcttctgtgcatttgcacggcctgtgcaaccgccctgttgttttttgttttcttttttctttttttgagacagtctcactctgtcgccaggctggagtgcagtagcgcgatctcgtctcactgcaacctccacctcctgggttcaagcgattctcctgcctcagcctcccgagtagccaggactacaggcgcgtgccaccacgcccagctaatttttctgtttttagtagagacgggctttcaccatattggccaggctggtctcgatctcctgacctcgtgatccgcccacctcggcctcccaaagtgctgggattataggcgcaagccaccgcgcctggcctgttgttattttttctaaagatgggggtctcgctattctgcccaggctggtctcgaactcctggcctcacgcaatcctcccacctcggcctctgaaggtgctgggattacaggtatgagccactgccccagacatcttgtatgttaaatgaagcaactaatcaactgtaacagccacagttgcggcccaccttggcttggtgtgcatattgcacagggcatggagtgagaacatcctggatctcaggatgccagggttcactgaggactctgactctagaaacaccttcattcctctggggtgagcaggggcaagcagcacttctctggaagcaaacaggtgctcctcctgaggtggggtcctctcccgtgggctggtcacagcctcgcctctgagctccgtcctcttccaccacctccaccaacagtggccagcagggctgcccccttggttgcccccctggtgacatattcttgtcaccacaggccgctgtgggttgctgccaaccaggtatcaggcaggctggagctacctggcagatgccctttgtgtagcctgccgagtggggccgccctcactggacccaagagcccctcacacatgggccttggtcggtcaggaccagccccatctcctacccagtctctcagaatattcatttgttgaatgcagtcgctggcagacacactctgtggccaggtaggcccagacctgtctgagtatggactgacagcaagctcagagaggtctggacctggccacggagcacctcatgc

SEQ ID NO: 105 - DNA, 4001 bp, Homo sapiens
  source 1..4001: mol_type = genomic DNA
acgagccacagactgacctgggatcttccaggccagcaggagtcttgcctccaagagcaccctctctgaggagaggatgccaggatttactggcaccttcagtatcctcctggcttcactcctctttgcccaaaaacaaaccacacctgtttctacctcccagcctttgcacttaccagtccccatgtctccccccatcccccacatgtggcatgccactagcctaccctcagtttcctaaaaggcactaagatctttccagcctcatggcctcaccacatgctgggagctctgcctggaatgcttttctttctactcttggccaattcgggccttggggcccagtaacacggttgagagatttactcctgtggccttgagagggctgaacaaatggaagcatctctaggttgcaccagaggcatctattacctgctgccctgtgcctgtaacttctgtcattctcctctccccctgtgagaacatgataaagaccacaaaggcagggaattcctctcctcttatgctgtgaccccacagctatctcgctccagctatgcctaagaaatctgtcttatcactgacatgcttgttgtccccactcgttccctgagcctccatccaacaagtcgctccaccacatgctggtggatctcgcttcctatgctcttccctagatcaggccttccctccattccctctaccactgccgtgccttgaggctcatcctctctcacatggatcccccgcccctacagcctccccactgccctcctgacctacagcctctcctgtccatttcccataccatggctaaggacacttaaaacccacctgaccagccatttccctactaaagctctcccaagacccggctcccccccaatatctcaagagtggcactcccaaacctctctcagttcaagcacccgcgcctctcacttcagcctcattttccacagttgcctgggctccagccactccagggcccccagccctgccccaaacatgctggcttttctaacactttatgcctatctttctgctttgccctcacaccttccatctgcaaaattcctattcattaatgggcaaaccattctggaatgctttctatgtgccagacactacctaggctctttttataccttatctcatttacttctcaaagtacctcacaaaaataataattattattcccattttacagatatggaaactgaggctcaagagaggcagggcaagaactggactcctagtctatctgattccaaacctgctgtgcaccaacctctctgggaagtgcaccagcgcccccactcccccagcccagggcagagtcttctctactctgtggttccatagccccctggtttctcgcttgtcccggccgttgtcaccagtgactgtgtgcggctgtctctgctgccccccagcgtgagcatcccaagggcagaacctgggctgatctggctgggtccccagcacccagcagggtataggtgctctgtgaggtttcttaataaagagatggaaagccagaagcagtttgggtggttgagatacccagccctaacctaactaatcctgataccagtgaccaaaagcgggaccttcgcatctttgctgccaaaagacagcccctcctaaagagtaaaggcccgaccccctgcacgccctctgcctgcaccgcacgtgcttggttttccccgcccgggtactggccgccgggccgtaccaatctccgcgggggagcgcccggggtcggactgagggagcgaggggaataaccgggcgcgccccttggaagcagggctcagagctgctctcctctcacgcattccccggatccgcgcggagcaggctgctggccagccccgggcccgcgccaagcagagcctcaggtgcggttcccccacaagcaagtggcgcgggcggcggcttagaacggcccgccccgcccgccgcgtcggcgcctgccccgttgtgaggtgataaagtgttgcgctccgggacgccagcgccgcggctgccgcctctgctggggtctaggctgtttctctcgcgccaccactggccgccggccgcagctccaggtgtcctagccgcccagcctcgacgccgtcccgggacccctgtgctctgcgcgaagccctggccccgggggccggggcatgggccaggggcgcggggtgaagcggcttcccgcggggccgtgactgggcgggcttcagccatgaagaccctcatagccgcctactccggggtcctgcgcggcgagcgtcaggccgaggctgaccggagccagcgctctcacggaggacctgcgctgtcgcgcgaggggtctgggagatggggtgagtgccacggcgcaggggttatggacctgcgagaagattttctggaaagggccctgtggcaggctggtgggtactgatgagtccacgttcattctccactgtggcactcatcaatttttacgacctctgttacatcgctttccacccgccccccagcttgtttccctcatccgtgaggtgggagcggtaccacccaccattcttagttattagggatattcgagaactcctccccagcccccactgcggctggtgacccctggcacttccctcccctctcccttcaccaggtagagcgagctttggcagtgatagactggatgggcaggatggtatgggttttgccgtctcctgagacagccaccagacggggaacatgcccgactggaacaggtgtgtctgccctgtcctctgtcccacatccatcctctcgcccaggctctggagcccacatagcagcaactcttgagcctggcatgcttagaaggcagcggagaggaccctgcatgtcctccaaggtagaactgaggtcctcagtgaatcgcgcagagttgaaatcaacccccgccccgcaccccccgcagcttttcccaagcgaggaatagcaactcttccaacccccacctcccttacctagagctggagaaactgaagtgggaggaagcatgcctaagttttccttagctgatgccttgacccctggattcaagtacaaatccggtagaccctgggaagtcatcacagctgtcctggtctgcgtgtgtgtcttgcatgaagcccctctccctctttctaagtctgtattctgtttgctgagcctctctgacatggatttttctttagtaactaacggtcgcctacaccgcccacctttgttacaaaaataaagcttgctaattatggaatttttgaaaatatagacaaatgaaaaaaatttctggtaaatcgactatccaaagataccttcctattagcattttagtatatctcttgatcattttctttatacacattgcatagatacaattgaggacatgctgtagatatagttatgaatcttgttttctcccccttaatgtaacatcgaggtttcccttctgttaatcagaatcacttacaatgtcctagtggttgcaataacccaccctgcagctgtaccacactttaactgtgacctaggcattggcattgcttcttgtgtgattatcgctgtggttatctgcccctcttggtgtgggtgctgctcgtagcccttgaagaggaacccagctgctgccctgtctcgggggcgagcagcttgagctgccccatgtatccagccagtagcctctgacagccccttctctcacttgagtccttttctgttccctgtgtcctttgatgtccttagggacatcaatggatgaatggacttgcccttgttcatgctgttaaaaatgtttttgcactgggcagtgggatagggatgttctctgtggtagtgcttcctggagaccccattccgtcggctctgccatccacaggctgggagctgtgtcttccaggaggcagtgaccctggctgtcatgtttttgacttagagtttgttccttaggagaacttgtactctagcgaatggttttaaccaagccacttaatatc

SEQ ID NO: 106 - DNA, 4001 bp, Homo sapiens
  source 1..4001: mol_type = genomic DNA
ccatcccccacatgtggcatgccactagcctaccctcagtttcctaaaaggcactaagatctttccagcctcatggcctcaccacatgctgggagctctgcctggaatgcttttctttctactcttggccaattcgggccttggggcccagtaacacggttgagagatttactcctgtggccttgagagggctgaacaaatggaagcatctctaggttgcaccagaggcatctattacctgctgccctgtgcctgtaacttctgtcattctcctctccccctgtgagaacatgataaagaccacaaaggcagggaattcctctcctcttatgctgtgaccccacagctatctcgctccagctatgcctaagaaatctgtcttatcactgacatgcttgttgtccccactcgttccctgagcctccatccaacaagtcgctccaccacatgctggtggatctcgcttcctatgctcttccctagatcaggccttccctccattccctctaccactgccgtgccttgaggctcatcctctctcacatggatcccccgcccctacagcctccccactgccctcctgacctacagcctctcctgtccatttcccataccatggctaaggacacttaaaacccacctgaccagccatttccctactaaagctctcccaagacccggctcccccccaatatctcaagagtggcactcccaaacctctctcagttcaagcacccgcgcctctcacttcagcctcattttccacagttgcctgggctccagccactccagggcccccagccctgccccaaacatgctggcttttctaacactttatgcctatctttctgctttgccctcacaccttccatctgcaaaattcctattcattaatgggcaaaccattctggaatgctttctatgtgccagacactacctaggctctttttataccttatctcatttacttctcaaagtacctcacaaaaataataattattattcccattttacagatatggaaactgaggctcaagagaggcagggcaagaactggactcctagtctatctgattccaaacctgctgtgcaccaacctctctgggaagtgcaccagcgcccccactcccccagcccagggcagagtcttctctactctgtggttccatagccccctggtttctcgcttgtcccggccgttgtcaccagtgactgtgtgcggctgtctctgctgccccccagcgtgagcatcccaagggcagaacctgggctgatctggctgggtccccagcacccagcagggtataggtgctctgtgaggtttcttaataaagagatggaaagccagaagcagtttgggtggttgagatacccagccctaacctaactaatcctgataccagtgaccaaaagcgggaccttcgcatctttgctgccaaaagacagcccctcctaaagagtaaaggcccgaccccctgcacgccctctgcctgcaccgcacgtgcttggttttccccgcccgggtactggccgccgggccgtaccaatctccgcgggggagcgcccggggtcggactgagggagcgaggggaataaccgggcgcgccccttggaagcagggctcagagctgctctcctctcacgcattccccggatccgcgcggagcaggctgctggccagccccgggcccgcgccaagcagagcctcaggtgcggttcccccacaagcaagtggcgcgggcggcggcttagaacggcccgccccgcccgccgcgtcggcgcctgccccgttgtgaggtgataaagtgttgcgctccgggacgccagcgccgcggctgccgcctctgctggggtctaggctgtttctctcgcgccaccactggccgccggccgcagctccaggtgtcctagccgcccagcctcgacgccgtcccgggacccctgtgctctgcgcgaagccctggccccgggggccggggcatgggccaggggcgcggggtgaagcggcttcccgcggggccgtgactgggcgggcttcagccatgaagaccctcatagccgcctactccggggtcctgcgcggcgagcgtcaggccgaggctgaccggagccagcgctctcacggaggacctgcgctgtcgcgcgaggggtctgggagatggggtgagtgccacggcgcaggggttatggacctgcgagaagattttctggaaagggccctgtggcaggctggtgggtactgatgagtccacgttcattctccactgtggcactcatcaatttttacgacctctgttacatcgctttccacccgccccccagcttgtttccctcatccgtgaggtgggagcggtaccacccaccattcttagttattagggatattcgagaactcctccccagcccccactgcggctggtgacccctggcacttccctcccctctcccttcaccaggtagagcgagctttggcagtgatagactggatgggcaggatggtatgggttttgccgtctcctgagacagccaccagacggggaacatgcccgactggaacaggtgtgtctgccctgtcctctgtcccacatccatcctctcgcccaggctctggagcccacatagcagcaactcttgagcctggcatgcttagaaggcagcggagaggaccctgcatgtcctccaaggtagaactgaggtcctcagtgaatcgcgcagagttgaaatcaacccccgccccgcaccccccgcagcttttcccaagcgaggaatagcaactcttccaacccccacctcccttacctagagctggagaaactgaagtgggaggaagcatgcctaagttttccttagctgatgccttgacccctggattcaagtacaaatccggtagaccctgggaagtcatcacagctgtcctggtctgcgtgtgtgtcttgcatgaagcccctctccctctttctaagtctgtattctgtttgctgagcctctctgacatggatttttctttagtaactaacggtcgcctacaccgcccacctttgttacaaaaataaagcttgctaattatggaatttttgaaaatatagacaaatgaaaaaaatttctggtaaatcgactatccaaagataccttcctattagcattttagtatatctcttgatcattttctttatacacattgcatagatacaattgaggacatgctgtagatatagttatgaatcttgttttctcccccttaatgtaacatcgaggtttcccttctgttaatcagaatcacttacaatgtcctagtggttgcaataacccaccctgcagctgtaccacactttaactgtgacctaggcattggcattgcttcttgtgtgattatcgctgtggttatctgcccctcttggtgtgggtgctgctcgtagcccttgaagaggaacccagctgctgccctgtctcgggggcgagcagcttgagctgccccatgtatccagccagtagcctctgacagccccttctctcacttgagtccttttctgttccctgtgtcctttgatgtccttagggacatcaatggatgaatggacttgcccttgttcatgctgttaaaaatgtttttgcactgggcagtgggatagggatgttctctgtggtagtgcttcctggagaccccattccgtcggctctgccatccacaggctgggagctgtgtcttccaggaggcagtgaccctggctgtcatgtttttgacttagagtttgttccttaggagaacttgtactctagcgaatggttttaaccaagccacttaatatcatgtcaggaacatttccccatgttgttatcagatcttgaaaacttttttttttttaaactgggtgcaaggatttacatcatggaatgtaggaagggctggtatgaaatgcaaaccagtcagttcagctttctgggatctactttggtgaaagattgggtggagtaggggagggcactgaagcacattttgttat

SEQ ID NO: 107 - DNA, 4001 bp, Homo sapiens
  source 1..4001: mol_type = genomic DNA
aactataaattcactaagataaatgccaccatttcccaagacttcctttttaaaaagcaatttaatcttatgcttgatgtatttctattatgcactggtaaatcaaaacaaaacaagcctggcgtggtggctcatgcctataatcccagcactttgggaggccgaggagggtggatcacctgaggtcaggagttggagaccagcctggccaacatggtgaaaccctgtctctaccgaaaatacaaaaattagccaggcgtagtggcgcacgcgtgtaatcccagctactcgggaggctgaggcatgagaatctcttgaaaccaggagacgaaggttgcagtgagccaagatcgcaccattgcactccagtctgggtgacagagcaagactccatctcaaacaaacaaacaaaacaacaacaacaaaattctacaacactagtaaacacagatggcttcataaattaatactttacctttggcatctttcatatgcccactttaaaatgtttaatattcttgggttcacatcagtcctcctgttatgcattcagttttactgagaaatgaagaaactgaggcaagagaagtgggaaaaaacttgctatttgtagcaagcaggcatctatctggacaggtaaactgtggtcctgctctattaggccacaatgcttttctgaaaagtgcctgtcaaataagcaccacagaaatccttctcatcacagacatccagggcatccccctcaagaagggacaaatatgagtattcttccctgctgagtgagaaaagaccatgtaaccaaagaaagcatgcttctcaatctatactaccgaattgggagggaaaaaatgaatggaaagagagagaaaatccaaggaaataaacttctgagactgaagatgtggttattaggtttatccacacattaggaaattactgaaacatcgcttaagtagaaacagaatcaggaaggcctagggtctttttttttattccagtcttaaacctccaaacaaaaatatctgaaattaaactatttctcctgagcagttatattcttaaatgtctctatagaacccacaaggcctacacgaagtccctgatttttagaggtgatttcaagtgtccattagtgtccactctcccactctcaaatccaaaataatgttggccccgatccgggcaggaccttcacgcacattttcctgtgaaatcattagcttcacatcagcttcctacgtcctatttccagtaacttcccttgatcagcatggcaaacgaggagagtaacaaaaggcatgtacatttactatgtccctcgcagagaacataataaaatcctgggaacaggaactggcagatacaacctgcctggccctggctctcgccacgatacaaaggcacggagagagcaagtgaggtctgcaccccacacctcactctgcaagagagaaaggactttcaatgcaatcagacccaacccgcaacgcccccttcccacgtggtcttctccctctcccgagcccagaatcggattggagaggggtctggaaggaatcagaccacgctggagctgggcaagaggggtggcgggcgagctccaggcagaaactccagccccccttcctagaggagccctgaactgtacgcctcccagttccgggatctgaatccccacctcccgagaccccagacctctccatgtctctagggaggggacctcaaggcctcagagctgcgggaagggctgggctgccccgcacccgatctggatagtgtggagcccaggccgcgtgcgggtgagcggaggctgggaggcggccccctgcctgccgccgcccctcccttggccccagtttctccaggtccccggtcacaggttctgggccggccctcacctcacctcagggtggcaacgtgtgcggtcgggcgattccggagcccctgcgtggaggaactgctgggcgggaggagacgccggcggctcgggcgatggctgacaggcgtgcagcaccaggccggcccggcacacggagaaggggcggggcttcaggggcctgacccgcccccgccccgcccctgtctcccacctcagccggcgagctccatgaggtgactacgtccggggttactccaggccgttcacctccgaccccgctccgggctgcggcgctgccagggccgccggggggcgaggcgcgggcccgagggctcagcggcagccaatgggagggcggcgcggggcggagcgatgtgaggcgatgccgatggtccgagcgcggatctgccattcggggcccagcggctagagggcgggcgggccgcgggcgggccgcgggcgggccctgggcagtcgtggcgcccctggcccggccttcgcggccccttctgccacagccgggtacggggccaacccctcggctcctcctcctggcggagaggggagtgggggaagcggtcccgcatgcgcctcgcatcagggacagaggcgtcccaagtgaggggccaggactagccggcctgtagaggcggggaaagggaacccagaattaaggcgggctagaatggggcaggcggtgtgcgggcccgtttgtctgtttaatcctaacacattaacaaggggtgggcaatcgcgtccactacctgtcctcatcacgctggctggtttccaagtctgggcgagctaagtaaccccatgctggaactccataaccagccccccgtttcgctcctcagccccgagacagcccactccaaaggtgttatacatgctggatcctggcgtgttgaggagaagcctcagtaaacgaagctcagtaagaaggggaatgtaccaaagaaccaccgtcttctgcctgccctgagcaaaatagttgccacagcgttccagacttttcggattggagaatgcttcccgtcctccactgtgggcacgccgagcctttgagaggaagccagtcggtgtctctagttttgccagttacttcatagcaagactttcatggtagctgtgtgttgaaaattaataataactggaaggggtcccaacacaatacaaacattgcgtagggaagaaacttggagaaaattgcacttgagaagaacagggtgaactcagcccttccaaggaggaatttaatgaagcatgcagcgcaccttcttaatgcttacaaacttgtgaggaaggccaggagaggtgcagaggatgacagtttggtaggaagaagttccacgccagagagtaaaaagtgaataggatagaaatacatgcccaggtaagaagttcttggcggaagctgaggcagaaagagggaaagattggtcaacaaggaacaatatttatctaggagccctcataatcccactaagtggggcttccctaagaatttatcaacccatttttaaaataatagggtcaagcacagtggcacgtacctggactgtagtcccacctactcagaaggctgaggtgggtggatggcttgaggcgaggagttcaagtctatatgtagtgtactgtgcctgcgcctgtgaatagccactgcaccccagcactggcaacgtaaaaagaccccatctctaaataaataagttaattaaataacagggattggaagcagagaacttaggaaagatgtggactttacagaacagggattcatatttcacaataattatatcactaatatatcaatcagttggacaaatgtagtgggaggaaaaagataattgggcaagacttcctggtttagtaagatgtttgatttgactcaccgaaaaaaatggcctgggaagagggaagccttgcctttccataacagcagtctctgaagctgacatgaagggagtgacttcaaaagaactaaaaaccatgcggttgcctcttacttttcttttacctttcctagatcctcttaagtaataggaattctttccccaggattcttttttttttttttttgagatggagtctcgctctgtcgcccaggctggagtgccgt

SEQ ID NO: 108 - DNA, 4001 bp, Homo sapiens
  source 1..4001: mol_type = genomic DNA
ctcccaaagtgatgggattacaggtgtgagccaccgctcccagcccacagtgatccttttacagcaaagctactcctttgctctcaacccttcgtcatattctgtcacttcccaataaaatccaaaccttaccagatgatacatgatctgccttgatttccatctaatttctctcatttgctgtgctccagccacactggtcttgccctttctccatgccaaacaaatccccatttcagggtcactgcacttgctgcttctcttctaggatagtttccctctagaaatccatgttgctccttctctcattaaatgatcagatatttatgtgcctccttagctaagaggccttccttatctaccctaactaagagaatccccatatcactcttatccttttatcctgatttatttttccataaaacttgccacattactgggcattatctatcatttagtaatgtgtatattgcttattgacagtcttccctactataatatgagctccatgaggcagagactttgttttgttcactgctgaattcccagcacctacaacaagcctactcactaaacatttgttgaattaatcttcaggatacatcaaatgtaaaatatcctatcatatatttattgatgtgatgcataggaaaatgtctggaagggtacataccaaaatgttaattatttatttaacctggtgggaggtggtttttctttccttttcttttctttttgagacagagtttcgctctcgttgcccaggctggagtggaatggcaagatcttggctcattgtaacctctgcccccagggttcaagcgattctactgcctcagcctccggagtagctgggattacaggtgcctgccaccacacccgcgtaattttttgtatttttagtagagacgggatttcaccatgttggtcaggctggtctagaactccagaccctgtgatccgcccgcctcggcctgccaaagtgctggaattacaggcgtgagccaccgcgcccggcagggcttttttttttttttttttttggaatgaacttgtattatgcatataccaaaaaagaatgaaagggttcacacaggcataaagtgtaagtgaattctaaaaaaaggttgggcaaatttatcaaaggcagacttctaaatagatgtaacagttatatagttcagataaaccatggtttgaattcccttccccagtacttcctagcagtgtgatcccaagttatttatgtcagtgcctagatttcttcacctggaaaatagggataacaacaggtgcctacctcacaagttttaaatgagaccgtatgtgtaacctgctttgtgcaatgtctggcatgcaataagtactcgacaaatggtagctattagaatcatcattaacagcagtaataagagatcttgggacatacctagccctcaagattgacatcagagtagaccaccacatcctctcatcattgcgcctcaatttgggcctctgaagcccaaagaggggatggttctttccaaagaagacaatttcgacgttccaggagcatctgattgaaacgcaccctcttcaccccttaaaatcagtctggttcatccacgagcagcccttcggggcccggactggagaggcgccacggctcgccgtccctgggcccagttccctcagcctcaatttcccttgctgcaacaggggtggagatgggaacgttatccccaaacccaggcagtcccggctcggcccgcctcaccgcactccggaggggaccaaacagccccacgcgccaggaagcctcaggcaacgggccacgcgccacacgggcaaagtgattactggtcggatcaaaagtcaggcaagcggctcagccccatcctcccagtcctcgggcacggggacagcagcaggcgcgcggcggggaccgggaaaaggccaagagggcggtggcgggcgctcagcacccggcgggggcctccacggccgaggggcggaggcggcgggcgcgcggcacgcggcgcgctcgccactgcgccgcccgggggcgcgcacgcaaagcccggaggcgcgcgcgaccggcggctctttggcgcggattagggggtctcggcgaggtgaggcgccaggcagcgctgggcgggcgggcgggcgggtggcggagcgcgagcccctctagcgggactagggagaagcgggggccacgaggtgcggcgggggaggagtcgcgggctgcgggccccggcggcgggcttgacgcggcaggctggcgggccggcgggcgggcagcggcgggcctcgccgctctgtgggggagggcggtgtggggccgggcctgaccccgggggcccgccccgcgtctgggcggtgccccagtgccccggcctcgccatttcccgctcagccctggagcgcgtagctctaccaagaatggccactgtgccagatgcccctgaccagcgttgcccatttgaatttcctagcaggccccccaaagtaggtatttcagtaccctgttagagctgaggcgcaggtaaaatgactggcccaggccggtcccaccccgtaaggatttgaacgttggctccacaactcgggagcctgcgcctttcctcctcccaacgtggactcctgcccggcgaagtgcctcacttccttctcccggtaactgattcgaatttggcgattgttagtttggagctagcgccagtgtttccacggaagttggtgtcagaatgtggtctcttgtgaaagggggagaaagaagattctgcagactgcaatattgtgcttttttgcctttttttttttttctcctaggctgtactgcagtgcagtggcacgatcgcggctcactgcaatctccgcctcccaggctcaagcgattctcgtgcctgaggcgtgggccaccacgcccagctaatatttgtattttcagcagaggtggggtttcaccatgttggccaggctggtctcgaactcctgacctcaagtgatccttccgcctcggcctcccaaagtgctgggattacaggcgtgagccaccacgcccggccctactatttttttattttttgagatggcgtttctttcgcccaggctggagtgcaatggcgccatctcaacacactgcaacttctgcctcccaggttcaagcgattctcctgcctcagcctcccgagtacctgggattacaggcgcccgccaccacacctggctattttttgtatttttagtagagatggggtttcgctgtgttggccaggttgatctcgaactcctgacctcaggttatctgcccacctgggactcccaaagtgctgagattacaggcgtgagccaccgcacccggcctcctactgtgctttttcgagtgttgatcagtccctattgactgtgcatcctttactggacgtctgtttttgtttttgttttttgagagggagtttcacacttgttgctcaggccagagtgcaacagcgcgttctcagcccaccgcaacctccacctcccgggttcaagcagttctcttgcctcagcctcccaagtagctggattacaggcatacgccaccacgcccagctaattttgtatttttagtagagacagggtttctacatgttggtcaggctgatctcgaactgccgacttcaggtgatctgcctgcctcggcctgctaaagtgctgggattacaggcgtgagccactgcgtccagccttgacatctgtatttgaaatgacacagctatcccttgtaagacactgcgcaagtaacatactctttcagagcttgcttgcttttgaaaaagcagcttgccccaaaagtgtgggagtgaggattcaaagaatccccacaccttggtacccacgggtacctagcaaattgaaaaagttcagtaaatttggcagttatttgattgctgcgttcgtaatacacccttgctagactcatttttcttcagagctcaacttttcctgagaggacagcaaagggagaccta

SEQ ID NO: 109 - DNA, 4001 bp, Homo sapiens
  source 1..4001: mol_type = genomic DNA
tgtaaagctcttagcacagtggctgtcataggccacacacatgccagtgacacagaaaaacacttgtcgcgacttattaggaatcacttcctcttccctgcaacactccctaacatttcctttcttcccttcttcaacccctatgtgggcattaaaaaaataatcctctgttgaaataactgaaaaaatggtctccctgggcccccaaacacacccagagtccttaggagcatcctcactggatcagggaacagataggatgttaattcagcctggtccatgctgctgaaagcccagagactttcccctttggaaaaatgcctgcccctgcagggcaccatcccctctgccccccaaaagcagttctaggctgcaggcctgggaagctgtccttgatggggggatctgcctgtcctgcctgggctggccgatccttagagagtggctctgacacctgaccaggactgacaggtgctgtgaaccaaatgtgaatcctttctgtccatgagtggaggaagatgctcacagcagaagccgccatcactatcacctgcctggttctttctgtttggtgcagcaggaaggggaaggctctatttggtgttgagtgactacatgaatcttggttgattctcacaataaggcaggtgaggtaggcgaggctgggtattttgcccccttttctagatggggacactgaggtttggagaggggaggtgatttgttcaagatcacactgtccattggtggcagagtcctggagcggtggctcacactgcccatggtgctctgagtggagatggtgaatgctgatggctcagatgtgtacagcccaactgcatccagcactgcactcagtgccccaggggggatgaactcattgaactcttaacagcccagcaatgatgtaggtaccatccttatccccattttgcagatgaggagactgatgatcagggagggcatttaacttgcccaaggcttccccagcttgtaagtggcagaaccgacatgtgaacccaggtcagtttccaggggaccaaacaaggtgcccggtgacagttgactttggccgccaatcatgcctcaatatagcggaagaactggcctggagccggggatcttggttggtcccagtctttggctctgtggctaattgcacatgtgacctcctggccaccatttgctcatctacaacacaagcacactggactcgatgatccctgtgcttccccgcattcagctgtaggttgcagcaggtggcttttgagaaacactctgatctgtgacaccccagatctacaggccgagtgcaggtgaaggtgtccaccagtcagcctcttacgccccagcagccacacttggcctgtccccatcagtttaattatctctgaatttgcaaaaggacattgcgccacttgctgaagggcccctgaccaccccctctttgtcccgatactgttgcttctctttcctgcctgggctgacctgtgtgtccagctggccattcgtgcccagtatagccaaacacaagttgtgctgagtctcaccctcccttctggacaggcgtctgacagatgaaccccttggaacatctgtcgatgctgttttcctgtctacccagggctgttgtgattacaggactgtttaaagcctgtaccagagatggagagttgggcaagagggcaattgcatgagcagtgaagtagggctaagtacagaaatgtggcaagctggagggaggtggtcctcaagcccacgaagcccctgggagcacagatagcctcttaactacctaagcttgaggtcaggagaaggctgagtgaggacctttgaaatggcttaaatttatgggtgacagataaaggcatgctgcactgccaacttgggggaacgctcacccatgtcggagtgctggccggtccaagccccacccccatggcctgatgcacgccaccagcctcccgctgaaaggtgacactctgccagctgggttcccttagtcaccctgtgggcgcctgtcagcctcactcaagaatggagctggtggctgcatccccagtgacggtaagtggcaacaagtgctggggagttaatcttggtgggaggaaaaatccaagcatggatggggattgctggatgctgacattctgtcccctctgtcctttaaaaacctcaccaggcatggggatgagtgtgctgtgggttgtggagggactggaaaacagactcctgactcccgtccttgccctgcccttgcctggtgactggccagcacttaccaggactgagcagtttctcgtgttcattgtggtgaaggccacgatcactctttcaacaggcagctgttgcttgggttaggtttgcaagacataaaaatagctttgtctggcatctggttttcgggtccgctgggttatttcagcgaagggcagcagcttgtcatgatagcgtccacagaggttcactggaagggctcgtctggccttccctgctcatggaatcaaactgcgtgatcggtgctcattcttgaccacggcccccccacgccaccgtctggcaccgttagtttgcatgtcgatctgcggctgtgtcttaaaggtacacagccaggtgtctgtctgtttttaggatcatatctacgcactccttgtaccacctgctgccttatggaatttacttatctccactgcagtggtgggacagtggaaaaggtatgagcaagggaaagttactgatccacagtttaccaagctgtaaaatggaagtcaaatagtgacaatgacaataataacatccattgggttgttcctttttttttttgagatggagtctcactctgtcacccaggctggagtgcagtggtatgatctcagctcactgcaacctctgcctcctgggtttaggtgattctcctgcctcagcctcccaagtagctgggattacaggcgtgcaccaccacgcccagctaatttttgtattttttagtagaatgaggtttcaccttgttggccaggctggtctcaaacttctgacttcaggtgatccacctgcctcagcctcccaaagtgctgggattacaggtgtgagccatcacgcccggtcccatggaggtgttttaaggattggagggaggtggtggtgagggtcttgggagcttgaagacactccacaaatttgatggcttgtatgtgtggttgctatataggtacagatgttatgcagagagcagcagtgaggtgaatggggtgagaattcgtatccccattttatagatgaggaaactgaggctcagagagtttcttctgtagctttctcaaacagtcctccgatttgtctctgtgtattcacttgggaaaagttcttgtgtggggtgacctttgggtcccacgtcattattccgcaaagaaaagggaaaggctgtgaggtttttcatgtaggtaaccctggcaaggaaggtttgggggattagatccaacagaataacagattgtaaaggatttgcagagtgagagaagggctgaactggggagaagagctggccactgcattcgtcagagcctccgctgtacaacttgcagctttacctgccatccctctatcacctgccgtgcgtgtgtctaaccatcttgcccattgactcatccactcaccttctctctccacacaccacctactcatccttctatccgtagttaatcagccatccatttgtccagccagccaggattttctaagcaagttcctgaagaacttatatcatgtaaaacttgcataatttgattggctttccaacaggaatcaatgtaaacaatgtattctcacacctcataattcacaaccattgtagcacacttttgtctttatgctgcacttctctttctctctctctctctctctctctctcgttgcccaggctggagtgcagtggcgtgatcttggctcactgcaacctccccctcctgagctcaagcaattctcctgcctcagcctcctgagta

SEQ ID NO: 110 - DNA, 4001 bp, Homo sapiens
  source 1..4001: mol_type = genomic DNA
tgtaatcccagtgctttaggaggcaaggcaagaagatcgctttaggccaggagttcaagaccaacttgggcaacataatgagaccttgtctttaaaaaaaaaaaatttttttttttttaattagccaggggtggtgtgtgcctgtagttctagctacttgggaagctgaggcaggaggatcatttgagcccaggagttcgaggctgcagtgagctatgatcatgccactgcactccatccagcctgggcaacagagcaagatcctatttctaaaacaaaacaaaacaaaacaaaaaatgtaacttgatacctgtgactttctgaaactaacagctgtttgaagaaatatatttctattaccgttcttgtgtttgatggaaagagtttatgaataactttggtttcaagccttccaattatattctggaaaatacctgtgtcttaaagagatagtgtctagggagaactgaatcactgcagagttcttcacctgtatgtgtgggtatgtctagtctttttgcaagtaatgaatttcattagaaagatattaagtgcttgtcatgagcaagcccctgtgccagaaactcaatataaatcatttcattttctcacccatatggtgaagtagttgatgaggacactgaagctcagccatttgctcaaagtcatggctaatggagggaggattcaaacccacatcagcttgatttcaaagcctaatagtcctgccactgtcaacaccctgcctagatcatgattatttggaatcactgggaaagaatttttttttcatgaagttagaagcctaagaatttttttcaatggaattttataaaaatgtccttgaatcacagatggctctccctggaaacataggtttgtttgagagtcacctcttttattgtcttaataaatagactaagagccatcacaagggctactgtggctagagaacatatgactcgggccgggcattgtgcaggcatttgacctgcattccttcaccccggttcctgtaacatgtacctggtgctgtggggttacagctgataatgctgaggtttccagagcacatgcttccaagtccagtggagaaaatcccaagaagtccaatattcaatgactgttccttattgcaaaataagcccagaaacaaagtagctgtgaacaggctgcaccagccccagcagcaatttccatttaaaccattttgcacagatcccgtgctcagcgctctcgccagtttctccacttgctgtttagtaaggccatcattgttggcaggaagtgggttccaccttccctgttgcctgagcacaatgtgctcctatgtggtctccactttcctgctgcctagttggctgggcttctccagctgggccttggaggcgacctttgtcccacatgccagccctccccacgttgcagaacactatgtaactgcagcgtgtgctgaatttcctcttctgtgctcaaccctagcaagagccagtcaaccctgaatgagaatgtggcaatggagctgtttttttcttttatccccaagtcacaagaaattggaaacattccattaagtggcgtgatttgagagggtttcgccaaatcttttcaggggagagaagccaggtgacctgatagcgatcaccttcccattcacggttttcaatgggggcttgtttctcaaatgaccaacacctgtaggcagttaattttcagcatgtcacaggagggcccagatgactttctgcctactgcttacgacaacatggactttagatgcctcatggcttcaacagctaagaattatgaaataaagcgagttatgggattaaaaggcagatagcagaactctcccaggagtagccagggctgcacaagtggtttgacgtgggatctttctcatctttgccccgggttgttgctgctttgaaggttaagtgctccagggatatttttaaacagcaggggctgattaatctaaaagtgacatttgcagtccccagagtaagcagctagcaggcttagattcaggccctcagcaaacaaggaacctggaaaatgtaaccctgaatgcacggtggggaggacatggcaagagaaaagcggcaggaataaagtggtaagtcagctcgcccctccccggggtccggaccattctgccaacgtccaggtaaattcgaacccaatgtatagtgagagcagaccctgtctgaaggcatgcacttggggtccccggggaaaaacgactcccgtcatgtttgaaggaagctaatcagaagagctcgaaatgtaacgcagatgaaagagaagggcctttctttgcttctataaatggtggtgtggatccctggacatgctcccatataagttccacaacgttttataaacaattctgttttttgcctccaaagctactgtcttaaggaatagtattctgtttctgtttcctcaaatggttgttctttttttttttttttttttttttaaagcagactaaggctgggcacggtggctcatgccacccagcaccttgggaggcagaggcaggtggatcacttgaggtcaggagttcgagaccagcctggccaacatggcgaaaccccatctgtactaaacacataaaaattagccagatgtggtgatgggcacctgtaatcccagctacttgggaggctgaggcaggagaatcgcttgaacccaggaggtgggggttgcagtgagctaaaattgcgccactgcattccagcctgactgacagagcaagactctgtctcaaaaaaaaaaaaaaagaaaagaaaaagaaaaaaaggagactaagagtaaagcgtttgtagtggtcctatatcctatatttctaggaagacatgactttctttcaatgttgaaaaaatgcattccaaaataatatgcaagttaaaaagtcactcagcaccgtgcaaaagtctccttgctgttattcctgtctaaccctcctcctccaggtagccactgtgcacattttggtataaaccccccagaccttttattctgtacactcacactgtacacaactctctccctcatgtctattgtgattacttttattttttaatatcaatgggcatccatataggttgggtgaccaaccatcccactttgcccaggaccggggaattcccgggacatgggacttgcaatttaaaaactgaaaggtgaagatattattattatcattattatttgagatggagtgctcactgcaagctccgcctcctgggttcacgccattctcctgcctcagcctcccaagtagctgggactacaggcacccactaccacgcccggctaattttttttgtatttttagtagagacagggtttcactgtgttagccaggatggtctcgatctcctgacctcaagtgatccgcccgcctcggcctcccagagtgctgggattacaggcatgagccaccgcgcctggccaaagatatcattttataaattgcatgtttctgctaatagtgacatggagttctttacaagatttctttttatgacttctccaagttttaagtggaaagacccagccaatcctaattcatgtgatttagagatgaaaatatctctaatctgtaactttctttcagaagctcttaagagttttatttgctcctgctcaggtgttgtgtaaactcgctcatactttcaacacctttaggtctgagacagaaatttgagtattacattacatgagatacccagtcttcatggggaaataaacattttaatcttttgaaatggaggtttccttgaagtcttatcccagtagaaactttgaccttgttctttttttttttttttttttgagacagagtctcgtcctgtcacccaggctggagtgcgatggcacaatctcagctcactgcaacccctgcctcccaggttcaagcaattcctcagtctcagtctcccgagtagctgggattgcaggcaagcactaccacgcccagctaatttttg

SEQ ID NO: 111 - DNA, 4001 bp, Homo sapiens
  source 1..4001: mol_type = genomic DNA
tcttcggggaacatggcgtgcccgtcgtggctacgttctgccaagccctgtgacgttggaggggagccgcctgcatcccccgctcagccagtgtttctagactccgagacatctggaactcggaagtgaggccagggctccaggaagactccctgatgacgcactggcccgcagcccaggctcaggtagtgggggctgtcaggatgatctgtgggatcccccagtgtccgaagaaagaagccacaattgtgtttttttttctttctttctttttctttttttttttttgagcgagtctcactctgtcgctcaggctgaagtacagtggcgcgatctcggctcactgcaacctctgcctccaggggtcaagcaatcctcccacctcagcctcccaagtagctgggattacgagcatgcactaccacgcccggctaatttttgtacttttagtagagaaacttttaactaaatgggcaagaaagaatgcatccagccagaacctcgagactagcaaatgcatttcttaaaagggcagggagggagaccagagtttgcttgaaaatactgcttcctttccttatctatgtagggaagggtttatcattctacattttgtggtcagcaagctggctttcacgggaggaggatgcatggtcgatttgccctctggtaactttgggatcttcctctaaacaacagtggactcccttagccaaggaagaagtgaagggataattgtgcttacaccagttagcttttcacacttctctctctcccggtgcagcactgccggtagggggagcagtgagctccaagtttccaggaataattcaaaacacaattattttatattttaaaatataaaatgtatattttaaatatatatacatacatttctatataatatatacaaaagttcaatatacgtatattgaatatatatgtatataagttcaatatatatgtatgtatattcaatatatatgtatgtatatatgtatatatattcaatatatatgtgtatatattgaactttagtcatgtggtgacactttaaacagtacacagagcagcttctgggtccatgaccctccaaaaatttaattactgattctttagaaactaggcaactcagccaggtgtgggggctcacgcctgtaatcccagcactttgggtggccgaggtgggtggatcacctgaggtcgggagttcaagaccagcctgaccagcatggagaaaccctgtctccactaaaaatacaaaattagccaggcgtggtggcacatgcctgtaatcccagctacttgggaggctgaggcaggagaatcacttgaacccgggcgatcgcaccattgcactccagactgggcaacaagagcataagagcaaaactccgtctcaaaaaaaaaaaaaaaaaaaaaccaaccaggcaactcattttgatgaactcttagctatgtggtagttgcttttttcatgtgtcaagtctgctatattcctgcactcagtatgcaaagatgcataaaaggcagtcttagccttttaggtcagcggttggcaaactttctgtgaagggtcaggtggtaaatatttgagggtttgcagttacccgttctccgtgggaactgctcaacaactcactgcagagaaagcagccgtgggcaaaacgtacaggaacgggcgaggctgtgttccaataaaactttattatttatggacactgaaattagattttaatatgattttcatgtgtcacgaaatctttttttttcctaagcatttaacaaatgtaaaaaccactcctagcttgggagctattggaaaacaggcatctggaaccacagtctgcagacccctgtgtgtactcactgtcagtgcggtgggaacaaaaaggcagcaacggctgggtcagcccaggaaagaagacatttcccagtggagagaatgtctgagtggatattgaactttccaggccacaaagaggaacaagagcattcaacactgaggagcctgcctctgcaaaggcaggacccacatgctggatgtggagctggatggttccacgtggcaaggtttgggaggcttctgagaacctggcctaatgattcctgtttcctcccttttcaggtgagttagggagatctctgacagcttttggaattggtaacacaaggaagtcagttattagcaaatgttgtactggaaacgtccagttataaaataattaattgtgtgaaagtcactgcctgcttttgatctacatccagcaggggaaggtcagggactataaattgtttgcattttttaaagatagactttagcatgttgctagtatgcttgcattattagataatacatctctgttcaaattcactttctgcttcagtttgcttctcatataaaagtcttcgcaagtatgtttggtggagaagcaaactttttggcgtatgcgtgtggtgttggcttttcatccttgccaaaccttcaagatttgcaaggaaaaacaggttgcagggaggtgctctgggttactttgaaaagacctcacatgctgacttgtgctgactcaagctgaggaaagctagaacattcctctgctggagatggcctccttgtttagagtccttctgacactgtaatttgggtctgacaacaggaagttcctttgtagtttaagtcaatgggaaccaatttttttttcttttttttgcgatagagtctcactctgtttcttaagatggagtacagtgactcagtcatggctcactgcagcctcaacttcctgggttcaggtgatcctcctgcctcagcctcctgagcagctgggaccactggtatgcaccaccatacctggctaagtttttaatagaaatggggtcttgctaagttgcccaggctagggaaccagtttttaagtttttgaaatatgcttggaaatataaatattggtgagcggagcagaccaggagatcaataatttatgttctctgcttgttagttgtgtatatagcatgttcttttgtctctcttaatgatgtttggggtgagggtgtgggagaggaaggtagggagccttccaacaagaagctagtgctcctccagcctagagaagctaaaacatgcaaagccagaccccatccctggaaattctgatttaattggtgggggaaggcatgtgaattctcacagagggtccggtttgcagcaggactggtgatttctttccaggtgccttgttttgttagaccatgtggtcacttaataattttttttgggccaggtacagtggctcatacctgtaatcccagtgctttaggaggcaaggcaagaagatcgctttaggccaggagttcaagaccaacttgggcaacataatgagaccttgtctttaaaaaaaaaaaatttttttttttttaattagccaggggtggtgtgtgcctgtagttctagctacttgggaagctgaggcaggaggatcatttgagcccaggagttcgaggctgcagtgagctatgatcatgccactgcactccatccagcctgggcaacagagcaagatcctatttctaaaacaaaacaaaacaaaacaaaaaatgtaacttgatacctgtgactttctgaaactaacagctgtttgaagaaatatatttctattaccgttcttgtgtttgatggaaagagtttatgaataactttggtttcaagccttccaattatattctggaaaatacctgtgtcttaaagagatagtgtctagggagaactgaatcactgcagagttcttcacctgtatgtgtgggtatgtctagtctttttgcaagtaatgaatttcattagaaagatattaagtgcttgtcatgagcaagcccctgtgccagaaactcaatataaatcatttcattttctcacccatatggtgaagtagttgatgaggacactgaagctcagccatttgctcaaagtcatggctaatggagggaggattcaaacccacatcagc

SEQ ID NO: 112 - DNA, 7680 bp, Homo sapiens
  source 1..7680: mol_type = genomic DNA
ctgaatgctccactttggaagggggagaatttagagggcaaaggggaatcgcacagggtcttaaagtgcaacagccacagtccttcctttttggggaaaaaaaaaaaaagtcccggccgggcatggtggttcacgcctgtaatcccagcactttggggaggccaaggcgagcggatcacgaggtcaagagatcgagaccatcctggccaatatgatgaaaccccatctctactaaaaatacaaaaattagctgggtgtggtggcacgcgcctgtagtcccagctacttgggaagctgaggcaggagaatcgcttgaacccgggaggcggaggttgcagtgagtcgagatcacgccactgcagcaagactctgtctcaaaaaaaaaaaaaaaaaatttaaaaagtcccaaatctgccaccatttattcttgatctttttcagaagcggcaccatgtactacagacggctcatccctcccctttgtcagtgtatagagggttctttggatgtagacacttttcaaagaccaatgagctgctgcagaagtctggcaagaagcccattgactggaaggagctgtgatcatcagctgaggggtggcctttgagaagctgctgttaacgtatttgccagttacgaagttccactgaaaattttcctattaattcttaagtactctgcataagggggaaaagcttccagaaagcagccatgaaccaggctgtccaggaatggcagctgtatccaaccacaaacaacaaaggctaccctttgaccaaatgtctttctctgcaacatggcttcggcctaaaatatgcagaagacagatgaggtcaaatactcagttggctctctttatctcccttgcctttatggtgaaacaggggagatgtgcacctttcaggcacagccctagtttggcgcctgctgctccttggttttgcctggttagactttcagtgacagatgttggggtgtttttgcttagaaaggtccccttgtctcagccttgcagggcaggcatgccagtctctgccagttccactgcccccttgatctttgaaggagtcctcaggcccctcgcagcataaggatgttttgcaactttccagaatctggcccagaaattagggctcaatttcctgattgtagtagaggttaagattgctgtgagctttatcagataagagaccgagagaagtaagctgggtcttgttattccttgggtgttggtggaataagcagtggaatttgaacaaggaagaggagaaaagggaattttgtctttatggggtggggtgattttctcctagggttatgtccagttggggtttttaaggcagcacagactgccaagtactgttttttttaaccgactgaaatcactttgggatattttttcctgcaacactggaaagttttagttttttaagaagtactcatgcagatatatatatatatatttttcccagtcctttttttaagagacggtctttattgggtctgcacctccatccttgatcttgttagcaatgctgtttttgctgttagtcgggttagagttggctctacgcgaggtttgttaataaaagtttgttaaaagtttgttttgtgcaagtgtcctttgtgcgtccaggccagggcatccatggacgtccttgggctgccctttcccttggcgcctcccagggttcccatagcaaccaccgtctgcaggaggggccgcccttgcccctcctccccgccctgccgctcagtggaacggcccaaccctcccctggctgcggtgagcgctgggcccaacccccggcctggagcagcgccccaactccgagcaccgtggagcaccggctgccagctgagaccccagaggggtaactaacggcctgaggaaggcatttcttcggggaacatggcgtgcccgtcgtggctacgttctgccaagccctgtgacgttggaggggagccgcctgcatcccccgctcagccagtgtttctagactccgagacatctggaactcggaagtgaggccagggctccaggaagactccctgatgacgcactggcccgcagcccaggctcaggtagtgggggctgtcaggatgatctgtgggatcccccagtgtccgaagaaagaagccacaattgtgtttttttttctttctttctttttctttttttttttttgagcgagtctcactctgtcgctcaggctgaagtacagtggcgcgatctcggctcactgcaacctctgcctccaggggtcaagcaatcctcccacctcagcctcccaagtagctgggattacgagcatgcactaccacgcccggctaatttttgtacttttagtagagaaacttttaactaaatgggcaagaaagaatgcatccagccagaacctcgagactagcaaatgcatttcttaaaagggcagggagggagaccagagtttgcttgaaaatactgcttcctttccttatctatgtagggaagggtttatcattctacattttgtggtcagcaagctggctttcacgggaggaggatgcatggtcgatttgccctctggtaactttgggatcttcctctaaacaacagtggactcccttagccaaggaagaagtgaagggataattgtgcttacaccagttagcttttcacacttctctctctcccggtgcagcactgccggtagggggagcagtgagctccaagtttccaggaataattcaaaacacaattattttatattttaaaatataaaatgtatattttaaatatatatacatacatttctatataatatatacaaaagttcaatatacgtatattgaatatatatgtatataagttcaatatatatgtatgtatattcaatatatatgtatgtatatatgtatatatattcaatatatatgtgtatatattgaactttagtcatgtggtgacactttaaacagtacacagagcagcttctgggtccatgaccctccaaaaatttaattactgattctttagaaactaggcaactcagccaggtgtgggggctcacgcctgtaatcccagcactttgggtggccgaggtgggtggatcacctgaggtcgggagttcaagaccagcctgaccagcatggagaaaccctgtctccactaaaaatacaaaattagccaggcgtggtggcacatgcctgtaatcccagctacttgggaggctgaggcaggagaatcacttgaacccgggcgatcgcaccattgcactccagactgggcaacaagagcataagagcaaaactccgtctcaaaaaaaaaaaaaaaaaaaaaccaaccaggcaactcattttgatgaactcttagctatgtggtagttgcttttttcatgtgtcaagtctgctatattcctgcactcagtatgcaaagatgcataaaaggcagtcttagccttttaggtcagcggttggcaaactttctgtgaagggtcaggtggtaaatatttgagggtttgcagttacccgttctccgtgggaactgctcaacaactcactgcagagaaagcagccgtgggcaaaacgtacaggaacgggcgaggctgtgttccaataaaactttattatttatggacactgaaattagattttaatatgattttcatgtgtcacgaaatctttttttttcctaagcatttaacaaatgtaaaaaccactcctagcttgggagctattggaaaacaggcatctggaaccacagtctgcagacccctgtgtgtactcactgtcagtgcggtgggaacaaaaaggcagcaacggctgggtcagcccaggaaagaagacatttcccagtggagagaatgtctgagtggatattgaactttccaggccacaaagaggaacaagagcattcaacactgaggagcctgcctctgcaaaggcaggacccacatgctggatgtggagctggatggttccacgtggcaaggtttgggaggcttctgagaacctggcctaatgattcctgtttcctcccttttcaggtgagttagggagatctctgacagcttttggaattggtaacacaaggaagtcagttattagcaaatgttgtactggaaacgtccagttataaaataattaattgtgtgaaagtcactgcctgcttttgatctacatccagcaggggaaggtcagggactataaattgtttgcattttttaaagatagactttagcatgttgctagtatgcttgcattattagataatacatctctgttcaaattcactttctgcttcagtttgcttctcatataaaagtcttcgcaagtatgtttggtggagaagcaaactttttggcgtatgcgtgtggtgttggcttttcatccttgccaaaccttcaagatttgcaaggaaaaacaggttgcagggaggtgctctgggttactttgaaaagacctcacatgctgacttgtgctgactcaagctgaggaaagctagaacattcctctgctggagatggcctccttgtttagagtccttctgacactgtaatttgggtctgacaacaggaagttcctttgtagtttaagtcaatgggaaccaatttttttttcttttttttgcgatagagtctcactctgtttcttaagatggagtacagtgactcagtcatggctcactgcagcctcaacttcctgggttcaggtgatcctcctgcctcagcctcctgagcagctgggaccactggtatgcaccaccatacctggctaagtttttaatagaaatggggtcttgctaagttgcccaggctagggaaccagtttttaagtttttgaaatatgcttggaaatataaatattggtgagcggagcagaccaggagatcaataatttatgttctctgcttgttagttgtgtatatagcatgttcttttgtctctcttaatgatgtttggggtgagggtgtgggagaggaaggtagggagccttccaacaagaagctagtgctcctccagcctagagaagctaaaacatgcaaagccagaccccatccctggaaattctgatttaattggtgggggaaggcatgtgaattctcacagagggtccggtttgcagcaggactggtgatttctttccaggtgccttgttttgttagaccatgtggtcacttaataattttttttgggccaggtacagtggctcatacctgtaatcccagtgctttaggaggcaaggcaagaagatcgctttaggccaggagttcaagaccaacttgggcaacataatgagaccttgtctttaaaaaaaaaaaatttttttttttttaattagccaggggtggtgtgtgcctgtagttctagctacttgggaagctgaggcaggaggatcatttgagcccaggagttcgaggctgcagtgagctatgatcatgccactgcactccatccagcctgggcaacagagcaagatcctatttctaaaacaaaacaaaacaaaacaaaaaatgtaacttgatacctgtgactttctgaaactaacagctgtttgaagaaatatatttctattaccgttcttgtgtttgatggaaagagtttatgaataactttggtttcaagccttccaattatattctggaaaatacctgtgtcttaaagagatagtgtctagggagaactgaatcactgcagagttcttcacctgtatgtgtgggtatgtctagtctttttgcaagtaatgaatttcattagaaagatattaagtgcttgtcatgagcaagcccctgtgccagaaactcaatataaatcatttcattttctcacccatatggtgaagtagttgatgaggacactgaagctcagccatttgctcaaagtcatggctaatggagggaggattcaaacccacatcagcttgatttcaaagcctaatagtcctgccactgtcaacaccctgcctagatcatgattatttggaatcactgggaaagaatttttttttcatgaagttagaagcctaagaatttttttcaatggaattttataaaaatgtccttgaatcacagatggctctccctggaaacataggtttgtttgagagtcacctcttttattgtcttaataaatagactaagagccatcacaagggctactgtggctagagaacatatgactcgggccgggcattgtgcaggcatttgacctgcattccttcaccccggttcctgtaacatgtacctggtgctgtggggttacagctgataatgctgaggtttccagagcacatgcttccaagtccagtggagaaaatcccaagaagtccaatattcaatgactgttccttattgcaaaataagcccagaaacaaagtagctgtgaacaggctgcaccagccccagcagcaatttccatttaaaccattttgcacagatcccgtgctcagcgctctcgccagtttctccacttgctgtttagtaaggccatcattgttggcaggaagtgggttccaccttccctgttgcctgagcacaatgtgctcctatgtggtctccactttcctgctgcctagttggctgggcttctccagctgggccttggaggcgacctttgtcccacatgccagccctccccacgttgcagaacactatgtaactgcagcgtgtgctgaatttcctcttctgtgctcaaccctagcaagagccagtcaaccctgaatgagaatgtggcaatggagctgtttttttcttttatccccaagtcacaagaaattggaaacattccattaagtggcgtgatttgagagggtttcgccaaatcttttcaggggagagaagccaggtgacctgatagcgatcaccttcccattcacggttttcaatgggggcttgtttctcaaatgaccaacacctgtaggcagttaattttcagcatgtcacaggagggcccagatgactttctgcctactgcttacgacaacatggactttagatgcctcatggcttcaacagctaagaattatgaaataaagcgagttatgggattaaaaggcagatagcagaactctcccaggagtagccagggctgcacaagtggtttgacgtgggatctttctcatctttgccccgggttgttgctgctttgaaggttaagtgctccagggatatttttaaacagcaggggctgattaatctaaaagtgacatttgcagtccccagagtaagcagctagcaggcttagattcaggccctcagcaaacaaggaacctggaaaatgtaaccctgaatgcacggtggggaggacatggcaagagaaaagcggcaggaataaagtggtaagtcagctcgcccctccccggggtccggaccattctgccaacgtccaggtaaattcgaacccaatgtatagtgagagcagaccctgtctgaaggcatgcacttggggtccccggggaaaaacgactcccgtcatgtttgaaggaagctaatcagaagagctcgaaatgtaacgcagatgaaagagaagggcctttctttgcttctataaatggtggtgtggatccctggacatgctcccatataagttccacaacgttttataaacaattctgttttttgcctccaaagctactgtcttaaggaatagtattctgtttctgtttcctcaaatggttgttctttttttttttttttttttt

SEQ ID NO: 113 - DNA, 4001 bp, Homo sapiens
  source 1..4001: mol_type = genomic DNA
tcatttgaataaaatcaacaacgaacagtccttttttttttctgttatgttctgatgtatattttactcacactcctagggcacttttaaaggtaagtttggaaagaggtctttctccaccagagtatgaaacagactagaacaaccaatcctacaacctgcagggcagtagccgctccactaattgattctccttctttgtctctaggaactatgcccaggcagaaaaaaagttactgtaggtgatgaagccagtgctccctgaaccaaataaaccctatcgacgttaccgaactgccgggcaaaaccagagcaactcacttacttggaaggtgaaaaacacttcaacatactccaggcggcaaccgacacttaggggccaggcagatgaaacaccatttgtttaaaaagtctattatttcactgtctcttcaacaaaggtggaaaactgatgattaaacactgaataatgccacccacttactaaacctatgattcactaataagcaggttcaatgcaattcataactttaaagaatgaattaccgaagccccttgcttcacaaattcccccaagaaacagaaagagcattcatcacatataacccgacaaccatttttactatggacacctacagatggcttctcttcctctaaggacacaatttccttcacaatcccacatgtaatcatttacaataccagaaacaaactccaactcagttccaccaactgggtttttaaaaatccttgtgcctaagaacgtcttttttccccaaagtaaaatgtgccattaaaaaatatttttaaaacgcaaaagtgcttatcattccgtacttcttttactaccttcagcagccaattattgacgtgataaaaggtagaaactcctggaatgcttcagagatcaacatgtgtcacagactcccagcctgtccgcctctaagacacagttggttcctttgttccttctgactgaacaccaactcctgatcaaaattgggaaactactttggaagggatgggagggtctgcagaatacatccacggaaaccttcataaaacaatattcaatcgtaataaggtggacacagaagaagggagaaaaggcagcctcccggacttccaccgggctcaccctaaatgggggccggcccctggctcctggtaaaatgatggctgaggcctcgggtccgtgagcctcgccatcgggaggggaccgcgagtgcgggcagagcggcccggggtcgggtgggggccgggcgagctgtcccttttcctattcactttcccagccgggtatccagcgagcaaggagcagggaggcacccaggcgaaaggcgccgcccaggatccggctggacagcaggacggagagcggcgaggcgagcggtgcccggggttcgggaccgagagggcaagcgagcgggggcaaccccgacccggccccccgagtccggccccgctcaggggccacgcgacggccgcccccttgcccagaactggcacggacgggcgggccgacccggggccactccacaacttgccggcccgccgccccggccccccgcgagaacccggagactcgaaccaggcagcagaggcaggatttccccggaaacgcgccctccccgccgccccgacgcccccgcggccctgccagccaagcgagagccgacgcgggccgcgctcccgggccgtttccggcggcgccggcggccgctactcctggcgccctggccgcggccgtcactcaccccaggccgccgcggctcccagcctccggctccgtcaggctcggtccgcgaaggcgcctgccgccccgtcctggcccggcgccccggcgagctcttccctccgaccagcggcgctcacggcgcagcggcggacgcgaggccacgggcggacgggcgcgcgcgggtggcgggcgggcgtggggccgggtcgtccctgcgcctcgccgccgcctcctccgccgccgcctcccgagttcggggctctgctgcaatgtctgcgaggctgactttgagcgcctcgctcaccgcccgcccgcagccgcgctcctccctccgccgccgagtccgccgcggcccagtccctcccctcgccgccccacgccattcagctcttcagccaagtttcttaacccttttctccccctccctcccattgtcacccggtcaggaggaagccgcgcggatccgggcggctcacaggagctgcggaaccccctctgtttcgtggatttccttctcgcctgctctccgggtgtcccagacggggctgcaagacccggaggcagcgagaggaccgcgtccccgagagaggggctgcacggcgcgcccgggccgtcccccgggctcccagctctgtccagccgcccgcgaccgctgcaggcgcctggacagctcctcctggggaccggggaaagcgctcttgggacccaggccgaatgctcccgtttgggcacagcctgggggccggggcgccccagacgctccagggtccccgcctgccttaagcgtctacctcctccccttcaccccacaacacgcgctctctgggccccctttcacccacccacccccaatttctatctccttgtaatctgtgtgtcctgctttaatgttggggggcacgggcgcgaactctgggtcactttgaccccacgcccagcacgtagtaggcgcccagcactcgtgtggagcgactctgggaggaaaactaggaaggagacctgaggagcgccccgctcagctccctggacagacgtaggacccaggagcagggttaatcctcggctgctttgtcactgcgaagtctccagcggagtctgtcagccaatctgcatcatccccgggaaggatctgacccaacaagacaagggaaaaggagcagggctgctacttcttcagcagctctcctcggttcggtctccgaataagaaataacaattacatcacgaggccggggcggcagcgcggtggagcctctcggtttcctgcagcctaatctcactgaacccgcctctaaattgggggggccacagattggtgaaacgagaaagtggaaggaggagagagtcatacgaaaccataatttccccttatccttgtatttgctgtttgaaaaatcgtatcctcaacgctccccagacatctcactagaactattaacaccccctcaacacctccccagatgtcaggtttctttttgacttgagcatctagcatttagtaattcgccatgaaaaaaattaccaggggaagcgagtgttgttgctgaaattgtttttgcttgacacaaaaaagctaaagtacagcttagtctcacccgaaattctgaaacggcatgcccgtgttaaaacacttattacataagacaaatgagccgatgaggcactccattcttatatgtaggttttcatatcctgaagaatttgatagcatttggaaatgaagttgctaatgcgcttaaaaaacaaaaactatccaagtcatgaaactgtattctgtgcctgttatgtaatggctgtattgtgtattgcaaatttcacttaacctttaaacaatatgaatgagagttttagaatactgtcacagtcttgaaagtgaaagcaatttttcaaattaatgcttaataaattcagcacccagcacagtgccgggcacatagtgccaatgttttccttagtggcaatggcacattttaaaacaattatttttcatcaccctagtgattgaactatgcaatctgttgccattttatatagttaaacctcatataatataataccatattatttgcatataccacaattcaaaatctgctctaggcaaaataaagtctgctgttttttagtgcatcatttattcagaattgatacaaagcaaaccgtttcaattcaatgatgaccagcataattggagcttggcccttagtggggggtcaaactttttttatgaaaattatttttaaccattatacagatttttctacaa

SEQ ID NO: 114 - DNA, 4001 bp, Homo sapiens
  source 1..4001: mol_type = genomic DNA
cacagttggttcctttgttccttctgactgaacaccaactcctgatcaaaattgggaaactactttggaagggatgggagggtctgcagaatacatccacggaaaccttcataaaacaatattcaatcgtaataaggtggacacagaagaagggagaaaaggcagcctcccggacttccaccgggctcaccctaaatgggggccggcccctggctcctggtaaaatgatggctgaggcctcgggtccgtgagcctcgccatcgggaggggaccgcgagtgcgggcagagcggcccggggtcgggtgggggccgggcgagctgtcccttttcctattcactttcccagccgggtatccagcgagcaaggagcagggaggcacccaggcgaaaggcgccgcccaggatccggctggacagcaggacggagagcggcgaggcgagcggtgcccggggttcgggaccgagagggcaagcgagcgggggcaaccccgacccggccccccgagtccggccccgctcaggggccacgcgacggccgcccccttgcccagaactggcacggacgggcgggccgacccggggccactccacaacttgccggcccgccgccccggccccccgcgagaacccggagactcgaaccaggcagcagaggcaggatttccccggaaacgcgccctccccgccgccccgacgcccccgcggccctgccagccaagcgagagccgacgcgggccgcgctcccgggccgtttccggcggcgccggcggccgctactcctggcgccctggccgcggccgtcactcaccccaggccgccgcggctcccagcctccggctccgtcaggctcggtccgcgaaggcgcctgccgccccgtcctggcccggcgccccggcgagctcttccctccgaccagcggcgctcacggcgcagcggcggacgcgaggccacgggcggacgggcgcgcgcgggtggcgggcgggcgtggggccgggtcgtccctgcgcctcgccgccgcctcctccgccgccgcctcccgagttcggggctctgctgcaatgtctgcgaggctgactttgagcgcctcgctcaccgcccgcccgcagccgcgctcctccctccgccgccgagtccgccgcggcccagtccctcccctcgccgccccacgccattcagctcttcagccaagtttcttaacccttttctccccctccctcccattgtcacccggtcaggaggaagccgcgcggatccgggcggctcacaggagctgcggaaccccctctgtttcgtggatttccttctcgcctgctctccgggtgtcccagacggggctgcaagacccggaggcagcgagaggaccgcgtccccgagagaggggctgcacggcgcgcccgggccgtcccccgggctcccagctctgtccagccgcccgcgaccgctgcaggcgcctggacagctcctcctggggaccggggaaagcgctcttgggacccaggccgaatgctcccgtttgggcacagcctgggggccggggcgccccagacgctccagggtccccgcctgccttaagcgtctacctcctccccttcaccccacaacacgcgctctctgggccccctttcacccacccacccccaatttctatctccttgtaatctgtgtgtcctgctttaatgttggggggcacgggcgcgaactctgggtcactttgaccccacgcccagcacgtagtaggcgcccagcactcgtgtggagcgactctgggaggaaaactaggaaggagacctgaggagcgccccgctcagctccctggacagacgtaggacccaggagcagggttaatcctcggctgctttgtcactgcgaagtctccagcggagtctgtcagccaatctgcatcatccccgggaaggatctgacccaacaagacaagggaaaaggagcagggctgctacttcttcagcagctctcctcggttcggtctccgaataagaaataacaattacatcacgaggccggggcggcagcgcggtggagcctctcggtttcctgcagcctaatctcactgaacccgcctctaaattgggggggccacagattggtgaaacgagaaagtggaaggaggagagagtcatacgaaaccataatttccccttatccttgtatttgctgtttgaaaaatcgtatcctcaacgctccccagacatctcactagaactattaacaccccctcaacacctccccagatgtcaggtttctttttgacttgagcatctagcatttagtaattcgccatgaaaaaaattaccaggggaagcgagtgttgttgctgaaattgtttttgcttgacacaaaaaagctaaagtacagcttagtctcacccgaaattctgaaacggcatgcccgtgttaaaacacttattacataagacaaatgagccgatgaggcactccattcttatatgtaggttttcatatcctgaagaatttgatagcatttggaaatgaagttgctaatgcgcttaaaaaacaaaaactatccaagtcatgaaactgtattctgtgcctgttatgtaatggctgtattgtgtattgcaaatttcacttaacctttaaacaatatgaatgagagttttagaatactgtcacagtcttgaaagtgaaagcaatttttcaaattaatgcttaataaattcagcacccagcacagtgccgggcacatagtgccaatgttttccttagtggcaatggcacattttaaaacaattatttttcatcaccctagtgattgaactatgcaatctgttgccattttatatagttaaacctcatataatataataccatattatttgcatataccacaattcaaaatctgctctaggcaaaataaagtctgctgttttttagtgcatcatttattcagaattgatacaaagcaaaccgtttcaattcaatgatgaccagcataattggagcttggcccttagtggggggtcaaactttttttatgaaaattatttttaaccattatacagatttttctacaatgtattgcaaatgtttctcttaagcagtttgttgaacgtcatttagcattttcttgataattcaatgttaattttattaaaagtaattattgtagtaggttgtagcggattcagtaaatatatccagttcctattctatattctttctaggtgttaggaatgctcccaggaggtacaaaagctatcagtcgctgcaccgaaggactctaggatgctatgacttagtgattatctctgccttttgtaatttctctctctcccccttcactaactgacaatctattgtccttactgttactagcatagctgccactaacattctcctcatcttctgatgggtaacttccagtctagtgcggactgggaatccagatagcctcacttgtcacaattctgtctaaagtaaacttaataggttctgggtaagggactaatgggctcaccaaggagtagggcgcacacacaccttagtgcatagactttgggaaagatatttggactactcaagtctatcttttaaatacattcctcttacttcttaaccaggttttggaccatggagattgcccagcaagccgagaatattccagccctccaacattgttgttaaaaagtccactgctgtatgttaggtgacttcccaaagctgctcactacttcttcttattctgttttctcaaccccacaaaaatccaatcactgaaatcctgaactcttttgatagtggcatgagtgttaagtaaattgcacacctcttgtggaattcattttcacttaatcatgctaattatgtggagacttgtttgttgttgttctttgtttttttaagtaaatcaatacctaaatttcctggctaacagagtcattgcaaggatactagggcattatatgtaatataagaatgcctatttggtaatttgtactttgaagtcaagtctcatttgaatc

SEQ ID NO: 115 - protein, 144 aa, Homo sapiens
MAATGANAEKAESHNDCPVRLLNPNIAKMKEDILYHFNLTTSRHNFPALFGDVKFVCVGGSPSRMKAFIRCVGAELGLDCPGRDYPNICAGTDRYAMYKVGPVLSVSHGMGIPSISIMLHELIKLLYYARCSNVTIIRIGTSGG

SEQ ID NO: 116 - DNA, 3016 bp, Homo sapiens
  source 1..3016: mol_type = genomic DNA
tgcagtaaagaagccagccaaaactggccaaaacctgccaaaaaccagcaaggtgaggaaaactacctctggtcttcctcattgctcattattcgctaattacaatgcattctcatgctaaacgacactcctaccagcaccatgacagtttataaataccatggcaacatttagaagttaccctatatgttctaaaaggaagaattcctagaaaactcatgaataatccacccttatttagtatatgatcaagaaataaccataaaaatagccaaccagcagccctagaggctgccctgcccatggaattgccattcttttattcctttactttctcaataagcttgcttctttcactttactctgtggactcaccctggccccgaattctttcttgcacaagatccaagaacattctcttggggtctggattgggacccttttccggtaacaaaactaaaactaagttgttaatctaccttcagagagagagaatagcaacttcctaaccaggccagtttcagtcttcagtctgaggcagcaaaatagggtctggaggcagggaacataaggccgattcacactttagctatgacaggaaatagcctctgcgtaggccaagtaaatgactttgtaactttgcatcatccctttcatttacatagtgtgtgccccaagcggaggacattgaaactccccaaaattctggaatggggcctttgagcccctacgctcaggcccgctcccacactgtggagtgtactttcatttaccttttttttttgagatggcgtctcactgtgtcaccaaggctggaatgcagtggcatgatctcagctcactgcaacctctgccacccaggttcaagtggttctcctgcctcagcctcccaagtagctggaattacaggcacctgccactgtgaccggctaggtttttttttttgttttttttttttttttacttttagtagagacggggtttcaccatcttggttaggctggtcttgaactcctgactttgtaatccacccgcctcggcctcccaaagggtttcaccatgttgggcaggctggtctcgaactcctgacctcaggtgatccatccgcctcggccttccaaagtgctgggattacaggcgtgagccaccactcccggcccaaccccatgttttctcaggaactggagttccctttaggaagcaagtaacagaggtgctgggctgcccgggcctctaggtcgtcttctttcccaggctctgtgctctcaccagagtgcgcgttcgggaagcagggagaggaccaggaaagacgcctgagacggagagtgggcaccggccgggggcaggcaggtccggcccctgggggtcagagtgcagcgccggtttgaggcagttcggccacttagctgcgactcgaagtgaatgagtcacttcccctcctcgggcgagctcctggctttgccccagcaagcgggaactcccaggatcaggaaggtcccagagaaactccccaggggggaagccgaggccgtccagagagatccagggtacacacagtgacagtgccagctccacattcccgaccaccgggccagggctggttccacgctgtcatttctctccgctggccccagccgcggggccaccgccagacagacagaccggggatcctgcctggcggcgtgaccttgggttagtcctagagccgcttctagcccggtttcctccgccccaggttaggaccacgtgggtaagaagatttcctctgtagtaggggctgtttaatgagtaacagaactggctgctggttctttacaattgcccagctgctctgcagaggcgagaagcaattgcgggctgagcctcccgggaccttcgcgggagggtggagaggaggaaggcgggggaggcggggccagaggggcagaggaaggtgggggcgggcggggcttgtctgcgggatggcccaggatgcggtcagctgagttcgccggcccagggcaggcggggcccgagcctagcggtaacccccgggcagggcggggccgctcgcagactccatatgagattcacctcgcaggtggttccctcattcgagtgctccggcgcacagacccgcgccccgccgtctgcgagcctcccgagagccgtcccttcgtccggccctggagcattgcgtttgtcgccggtgtcgcagtgcgaggatggcgccgcgggtgtagcggctctctgcgcaggccgagtggtgagtcgcccgcggcccggctcccgccgttctctcttcccaggggtctagcccgtcccctgccttggccacgtgcccatttacctgtaggggcggtgacggaggctctggtgcaccttggctgtgagctgcagaggagcagagagcggcccggcgcggctgcgcggggaggcgggcgcgcggccgggaggttctggggctggtgtgtgcggagcgaggtgggcgtgggggaagcgcgcgggcgggatgtgcttcctcgggtccaccccgtgcctacctctcccacgtcccggcgacggagcgcgcggggctctgggcggggagcccaccgggccgaggcgcagcgagggtgaagccagagccggcagggtcaggtcccgcagggcgcaggcgggcgcttctcgcagcccaaacccgggcggggagtgcgggaacccctagaactatgctgggagccccagataagaggaaggagatggtgacttaagtgacgaagtgatgttttgtccccaccgccttttaaagggagaagggggtaaaatttcatttggaggatgattgttacgcgagctttcagagttcagatggcgtcaagtggccatcgagtcaacatttaacaaaatgttaccgcataaaatattgtcatcgagttatctggcagctccgcagagaaacttcacaggcatatgcgttatatgacataatttgcatggggaagggggtcccttcacgattcttttatatggcaacctgttcttggacctggcaggg

SEQ ID NO: 117 - protein, 381 aa, Homo sapiens
MPFSNSHNALKLRFPAEDEFPDLSAHNNHMAKVLTPELYAELRAKSTPSGFTLDDVIQTGVDNPGHPYIMTVGCVAGDEESYEVFKDLFDPIIEDRHGGYKPSDEHKTDLNPDNLQGGDDLDPNYVLSSRVRTGRSIRGFCLPPHCSRGERRAIEKLAVEALSSLDGDLAGRYYALKSMTEAEQQQLIDDHFLFDKPVSPLLLASGMARDWPDARGIWHNDNKTFLVWVNEEDHLRVISMQKGGNMKEVFTRFCTGLTQIETLFKSKDYEFMWNPHLGYILTCPSNLGTGLRAGVHIKLPNLGKHEKFSEVLKRLRLQKRGTGGVDTAAVGGVFDVSNADRLGFSEVELVQMVVDGVKLLIEMEQRLEQGQAIDDLMPAQK

SEQ ID NO: 118 - DNA, 1815 bp, Homo sapiens
  source 1..1815: mol_type = genomic DNA
aagaccccggccccgcccgcccggcccgcccgcagccccgcacctgcaggttgtcggggttgaggtcggtcttgtgctcatcgctgggcttgtagccgccgtgccggtcctcgatgatggggtcgaagagatccttgaacacttcgtaggactcctcgtcgcccgccacgcagcccacggtcatgatgtacgggtggcctgggggagggggcgcgggacggggacagtgacgtcactgccgggcccgagcgcgctgctgaggaccctgcggctgcgcggggggagggggggccgggaccccggccccgaggggtcgcgtacccgggttgtccacgcctgtctggatgacgtcgtccagcgtgaagccgctcggcgtgctcttggcgcgcagctccgcgtacagctcgggggtcagcaccttggccatgtggttgttgtgggcgctcaggtcggggaactcgtcctcggccgggaagcgcagcttcagtgcgttgtggctgttggagaagggcatggcggcggcgggctgcggggagacgcggggtcagaggggaccggcacgccggggttcccgggctcccgcgtaccactcaggcccccgccgccgggccccccggcgccccccgggacgcggccaaggtcagcggggtccgcagcgcgcgcggggagcgccatcatcgccggggacccccggccggtccggcgcgcgctccaggcgacgcgccccctttgcacgcagcgcccctagcccgggcgcggacgcccccggcccccgggcccactcaccgggcggccgggcgggggcgggggcgctccgtccgtcggcagctcccggagcgacgcaggcgaacagcggccgctccgcgctcccgcggctcttaaggggcacaacgcgggccgcccattggcggctatttatagcccattcattccattggcccgcgccgcggggtggtgccgccgctttgtccgccgcccgcagcccccaacaccctggccgcccacccgagctgtcccctcgctgggaccccgcgcagcctgcccagtcccccgcccaagacctcgaggccgaaacctcaggtccttggatatctggatcccctcgcacgcccttgcctctgggcgggaaactgaggcggagagcctccgggcactcgaggacgcttgggttcatccgcatcctcgagcctgcccgcctagtccagccagcccccgcggcggcggcgaggggccgggcgaaagctttcggggcttccgcagcaggtacggcccccgccccacccccggccagtgcgagctggcccggagccgccgaccttcctcaaggtgcccgcaggcgtttcctgggcgatcctggtgcaaagcgaaggcgcgcgggaagcgcagtctggctgcggcccagcccgggggcacccgcggtgccctgaccttggacaagtcctgggtctcggggggctgcgagccagaaaccacggggacgctgtggccttaagaggccgctggcaagtgcagcctcggagtttccagaccccaaccctgggccctcttcgcaggggacaagtttacccgacccccaaaggcagtccccggagacttcaggcaaagatgggaggaaacgctaataaacaataagcataagtgcgattaaaaaaaaaaactgatctcacgaataaccagaatcatgcaaattaagcagtgactccacctgcccctacccccaacactcccagatcgaccccagccattgctggctttgagctggaatgcgggaagacaggcgtccacacgctgcctgcaacttaaattgctgcatgc

SEQ ID NO: 119 - DNA, 22 bp, synthetic construct
  source 1..22: mol_type = other DNA
gtctcctctgacttcaacagcg

SEQ ID NO: 120 - DNA, 22 bp, synthetic construct
  source 1..22: mol_type = other DNA
accaccctgttgctgtagccaa

SEQ ID NO: 121 - DNA, 19 bp, synthetic construct
  source 1..19: mol_type = other DNA
tggaatagcggcgtgcttg

SEQ ID NO: 122 - DNA, 19 bp, synthetic construct
  source 1..19: mol_type = other DNA
ctcatcagattgggagtag

SEQ ID NO: 123 - DNA, 22 bp, synthetic construct
  source 1..22: mol_type = other DNA
ccaaaggatgcgctctcgttca

SEQ ID NO: 124 - DNA, 22 bp, synthetic construct
  source 1..22: mol_type = other DNA
cggtgtctgtagtggcttgact

SEQ ID NO: 125 - DNA, 21 bp, synthetic construct
  source 1..21: mol_type = other DNA
caggccgtgatggcagagatg

SEQ ID NO: 126 - DNA, 23 bp, synthetic construct
  source 1..23: mol_type = other DNA
ggtttcaccgatgtctcccttag

SEQ ID NO: 127 - DNA, 22 bp, synthetic construct
  source 1..22: mol_type = other DNA
gcggaatttgctgccaacactc

SEQ ID NO: 128 - DNA, 22 bp, synthetic construct
  source 1..22: mol_type = other DNA
agacttctgggcttgctggtgt

SEQ ID NO: 129 - DNA, 23 bp, synthetic construct
  source 1..23: mol_type = other DNA
acgagaggatgataaactggtgg

SEQ ID NO: 130 - DNA, 22 bp, synthetic construct
  source 1..22: mol_type = other DNA
gcgaacttcagtccaggtcaac

SEQ ID NO: 131 - DNA, 22 bp, synthetic construct
  source 1..22: mol_type = other DNA
gaaagagtggcaacctgccttc

SEQ ID NO: 132 - DNA, 24 bp, synthetic construct
  source 1..24: mol_type = other DNA
gcaccaagttttactacatctgcc

SEQ ID NO: 133 - DNA, 23 bp, synthetic construct
  source 1..23: mol_type = other DNA
caggtcaacctattggtcaagcc

SEQ ID NO: 134 - DNA, 22 bp, synthetic construct
  source 1..22: mol_type = other DNA
gccttctcatcaccaatggtcc

SEQ ID NO: 135 - DNA, 20 bp, synthetic construct
  source 1..20: mol_type = other DNA
tgggcttcttcatcttcacc

SEQ ID NO: 136 - DNA, 19 bp, synthetic construct
  source 1..19: mol_type = other DNA
gtgctgggtttcacctcct

SEQ ID NO: 137 - DNA, 22 bp, synthetic construct
  source 1..22: mol_type = other DNA
catctccgacttcctggacaag

SEQ ID NO: 138 - DNA, 22 bp, synthetic construct
  source 1..22: mol_type = other DNA
tggtccagttgagaaggatgcc

SEQ ID NO: 139 - DNA, 22 bp, synthetic construct
  source 1..22: mol_type = other DNA
tgtatcgccgtaaactggaccg

SEQ ID NO: 140 - DNA, 22 bp, synthetic construct
  source 1..22: mol_type = other DNA
tgtctgagaggtgctgtagcac

SEQ ID NO: 141 - DNA, 19 bp, synthetic construct
  source 1..19: mol_type = other DNA
tgcaccaccaactgcttag

SEQ ID NO: 142 - DNA, 19 bp, synthetic construct
  source 1..19: mol_type = other DNA
ggatgcagggatgatgttc

SEQ ID NO: 143 - DNA, 21 bp, synthetic construct
  source 1..21: mol_type = other DNA
gtgaacccgacaacttccgaa

SEQ ID NO: 144 - DNA, 21 bp, synthetic construct
  source 1..21: mol_type = other DNA
tgaaactccggctgagaagat

SEQ ID NO: 145 - DNA, 20 bp, synthetic construct
  source 1..20: mol_type = other DNA
acagctttctgggtggattg

SEQ ID NO: 146 - DNA, 20 bp, synthetic construct
  source 1..20: mol_type = other DNA
tgtctctgtgaggaccgcta

SEQ ID NO: 147 - DNA, 23 bp, synthetic construct
  source 1..23: mol_type = other DNA
tatggagtgacatagagtgtgct

SEQ ID NO: 148 - DNA, 22 bp, synthetic construct
  source 1..22: mol_type = other DNA
ccacttcaatccacccagaaag

SEQ ID NO: 149 - DNA, 21 bp, synthetic construct
  source 1..21: mol_type = other DNA
gtaacttcattgtcggcatgg

SEQ ID NO: 150 - DNA, 20 bp, synthetic construct
  source 1..20: mol_type = other DNA
agctgagatctggtcaaacg

SEQ ID NO: 151 - DNA, 23 bp, synthetic construct
  source 1..23: mol_type = other DNA
gcatctctgacttcctggacaag

SEQ ID NO: 152 - DNA, 22 bp, synthetic construct
  source 1..22: mol_type = other DNA
cttggtccagttgagcaggatg

SEQ ID NO: 153 - DNA, 24 bp, synthetic construct
  source 1..24: mol_type = other DNA
ggacattgagattcttttcctctg

SEQ ID NO: 154 - DNA, 23 bp, synthetic construct
  source 1..23: mol_type = other DNA
gcaaaggcattggctggaagaac

SEQ ID NO: 155 - DNA, 22 bp, synthetic construct
  source 1..22: mol_type = other DNA
atgtatcgccgcaaactggacc

SEQ ID NO: 156 - DNA, 22 bp, synthetic construct
  source 1..22: mol_type = other DNA
ctctgagaggtgctgtagcaag

SEQ ID NO: 157 - DNA, 22 bp, synthetic construct
  source 1..22: mol_type = other DNA
gaggagaaacgccaaccaagag

SEQ ID NO: 158 - DNA, 22 bp, synthetic construct
  source 1..22: mol_type = other DNA
tttgtcctcggtggcgttatcc

SEQ ID NO: 159 - DNA, 22 bp, synthetic construct
  source 1..22: mol_type = other DNA
agcatcggatgcttaggagtgg

SEQ ID NO: 160 - DNA, 22 bp, synthetic construct
  source 1..22: mol_type = other DNA
cagccagtcatctttggagacc

SEQ ID NO: 161 - DNA, 20 bp, synthetic construct
  source 1..20: mol_type = other DNA
acgtccactctgcgagaatg

SEQ ID NO: 162 - DNA, 21 bp, synthetic construct
  source 1..21: mol_type = other DNA
cgatcaggctgtacgtcatct

SEQ ID NO: 163 - DNA, 22 bp, synthetic construct
  source 1..22: mol_type = other DNA
acaaggtagagctggagtctcg

SEQ ID NO: 164 - DNA, 22 bp, synthetic construct
  source 1..22: mol_type = other DNA
agcaccacagatgtgtccgaga

SEQ ID NO: 165 - DNA, 22 bp, synthetic construct
  source 1..22: mol_type = other DNA
atcctgctggatgtgaagacgc

SEQ ID NO: 166 - DNA, 22 bp, synthetic construct
  source 1..22: mol_type = other DNA
tccacaatggtacgcacctgac

SEQ ID NO: 167 - DNA, 22 bp, synthetic construct
  source 1..22: mol_type = other DNA
ctccaacgtcactatcatccgc

SEQ ID NO: 168 - DNA, 22 bp, synthetic construct
  source 1..22: mol_type = other DNA
ctgccttgaagcaggtatccac

SEQ ID NO: 169 - DNA, 22 bp, synthetic construct
  source 1..22: mol_type = other DNA
atcaccccccaccagatcaagc

SEQ ID NO: 170 - DNA, 23 bp, synthetic construct
  source 1..23: mol_type = other DNA
gtccttgtcgtacaggccgttca

SEQ ID NO: 171 - DNA, 21 bp, synthetic construct
  source 1..21: mol_type = other DNA
gggacttggcaggggcgtgcc

SEQ ID NO: 172 - DNA, 21 bp, synthetic construct
  source 1..21: mol_type = other DNA
ctgagccggcccaggtctcca

SEQ ID NO: 173 - DNA, 21 bp, synthetic construct
  source 1..21: mol_type = other DNA
caggtctccaaagagctgcta

SEQ ID NO: 174 - DNA, 21 bp, synthetic construct
  source 1..21: mol_type = other DNA
ctttgggagtgacgcgcggct

SEQ ID NO: 175 - DNA, 21 bp, synthetic construct
  source 1..21: mol_type = other DNA
ggctttgggagtgacgcgcgg

SEQ ID NO: 176 - DNA, 21 bp, synthetic construct
  source 1..21: mol_type = other DNA
gcgaactcagctgaccgcatc

SEQ ID NO: 177 - DNA, 21 bp, synthetic construct
  source 1..21: mol_type = other DNA
gggcttgtctgcgggatggcc

SEQ ID NO: 178 - DNA, 21 bp, synthetic construct
  source 1..21: mol_type = other DNA
gatggcccaggatgcggtcag

SEQ ID NO: 179 - DNA, 21 bp, synthetic construct
  source 1..21: mol_type = other DNA
ggtcagctgagttcgccggcc

SEQ ID NO: 180 - DNA, 21 bp, synthetic construct
  source 1..21: mol_type = other DNA
agttcgccggcccagggcagg

(19) United States
(12) Patent Application Publication
(10) Pub. No.: US 2026/0272991 A1
(43) Pub. Date: Sep. 17, 2026

(54) WHITE FAT AND ADIPOSE ORGANOID MODULATION AND THERAPEUTIC TRANSPLANTATION

(71) Applicant: The Regents of the University of California, Oakland, CA (US)

(72) Inventors: Nadav Ahituv, Walnut Creek, CA (US); Hai Nguyen, Austin, TX (US)

(21) Appl. No.: 19/469,191

(22) PCT Filed: Mar. 27, 2024
(86) PCT No.: PCT/US2024/021744

§ 371 Date: Sep. 25, 2025

Related U.S. Application Data
(60) Provisional application No. 63/455,129, filed on Mar. 28, 2023.
(60) Provisional application No. 63/594,225, filed on Oct. 30, 2023.

(51) Int. Cl. A61K 35/35; A61P 35/00; C12N 5/077; C12N 9/22; C12N 15/11; C12N 15/86

(52) U.S. Cl. CPC A61K 35/35; A61P 35/00; C12N 5/0653; C12N 9/226; C12N 15/111; C12N 15/86; C12N 2310/20; C12N 2510/00; C12N 2513/00; C12N 2750/14143

(57) ABSTRACT

Methods and compositions for controlling fat cell phenotypes are provided.

### CROSS-REFERENCE TO RELATED APPLICATIONS

This application is a U.S. National Phase Application of PCT/US2024/021744, filed Mar. 27, 2024, and which claims priority to U.S. Provisional Application No. 63/455,129, filed Mar. 28, 2023, and U.S. Provisional Application No. 63/594,225, filed Oct. 30, 2023, the disclosures of which are herein incorporated by reference in their entirety for all purposes.

### STATEMENT AS TO RIGHTS TO INVENTIONS MADE UNDER FEDERALLY SPONSORED RESEARCH AND DEVELOPMENT

This invention was made with government support under R01 DK124769 awarded by the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Oct. 6, 2025, is named 081906-1525020-260710US_SL.xml and is 367,602 bytes in size.

### BACKGROUND OF THE INVENTION

Tumors are complex tissues composed of cancerous and non-cancerous cells in a hypoxic and nutrient-deprived microenvironment. The tumor microenvironment (TME) contains heterogeneous cell populations, including immune cells, mesenchymal support cells, and matrix components contributing to tumor growth and progression (1). In order to survive this environment, tumors are capable of reprogramming metabolic pathways to better utilize available substrates in the surrounding TME, ultimately becoming dependent on these pathways for continued growth and survival (2). In contrast to normal cells, the main pathway of glucose metabolism in cancer cells is aerobic glycolysis, termed the Warburg effect (3). Glucose uptake and production of lactate is increased in these cells, even in the presence of oxygen and functional mitochondria (3). The increase in glycolytic flux allows glycolytic intermediates to supply subsidiary pathways to fulfill the metabolic demands of proliferating cells. During hypoxia, cancer cells also undergo metabolic reprogramming to increase lipid utilization, as fatty acids produce twice the energy of glucose (4, 5).

There are many efforts to target cancer glucose and fatty acid metabolism for therapeutic purposes. For glycolysis, these include drugs that target hexokinase 2 (HK2), which is involved in the initial steps in glycolysis, using ATP from the mitochondria to phosphorylate glucose, or drugs that target glucose transporters (GLUT1 and GLUT4) (2, 6-8). Several drugs are also used to target lipid metabolism in cancer (9-12). These include drugs that target lipid uptake (targeting proteins such as LXR, CD36 and FABP4/5), lipogenic enzymes (such as ACC, ACLY, FASN, and SCD1) and proteins involved in intracellular lipid homeostasis (such as CPT1A, PPARG and others) (13, 14). In addition, recent work has shown that cold activation of brown adipose tissue (BAT), which dissipates energy by non-shivering thermogenesis, increases adipocyte glucose uptake, lipid metabolism and significantly inhibits tumor growth (15). However, situating cancer patients in cold conditions for extended periods is challenging.

### BRIEF SUMMARY OF THE INVENTION

In some embodiments, a composition is provided that comprises a targeted polypeptide linked to a transcriptional activator, wherein the targeted polypeptide targets a portion of a promoter or enhancer sequence operably linked to a polynucleotide encoding a polypeptide selected from the group consisting of Uncoupling protein 1 (UCP1/SLC25A7), PPARG coactivator 1 alpha (PPARGC1A/PCG1A), PR/SET domain 16 (PRDM16), FFAR1/GPR40, FFAR4/GPR120, FOXO1, GLUT1, GLUT2, GLUT3, GLUT4, CHREBP, CD36, CTP1B, AIFM2/FSP1, GSK, IRS1, DGAT1, DGAT2, UPP1, CKB, and ACACA or as found in Table 2, wherein the composition upregulates expression of the at least one polypeptide when introduced to a cell. In some embodiments, the targeted polypeptide is a catalytically-inactive nuclease. In some embodiments, the catalytically-inactive nuclease is derived from (i) a zinc finger nuclease (ZFN); a transcription activator-like effector nuclease (TALEN); (iii) a clustered regularly interspaced short palindromic repeats (CRISPR) nuclease. In some embodiments, catalytically inactive nuclease is derived from a clustered regularly interspaced short palindromic repeats (CRISPR) nuclease, wherein the composition further comprises a gRNA that targets the portion of the promoter or enhancer sequence. In some embodiments, the gRNA comprises a sequence having at least 90%, 95%, 98%, 99% or 100% identity to a sequence of any one of SEQ ID NOs: 1-55. In some embodiments, the targeted polypeptide is a catalytically-inactive nuclease dCas9.

In some embodiments, the transcriptional activator is selected from the group consisting of HSF1, VP16, VP48, VP64, VP160, p53, p65, RTA, MyoD1, SET7, LSD1, CIB1, AD2, CR3, GATA4, SP1, MEF2C, TAX, SET9, VP64-p65-RTA (VPR), Histone Acetyltransferase p300, TET1 Hydroxylase Catalytic Domain, Synergistic activation mediator (SAM), SunTag, and PPAR-gamma. In some embodiments, wherein the transcriptional activator is VP64. In some embodiments, the transcriptional activator is VP64, and the targeted polypeptide is catalytically-inactive nuclease dCas9.

In some embodiments, a composition is provided that comprises a targeted polypeptide linked to a transcriptional repressor, wherein the targeted polypeptide targets a portion of a promoter or enhancer sequence operably linked to a polynucleotide encoding a polypeptide selected from the group consisting of ACACB and NRIP1, wherein the composition downregulates expression of the at least one polypeptide when introduced to a cell. In some embodiments, a cell comprises the targeted polypeptide. In some embodiments, a polynucleotide comprises one or more expression cassettes encoding the targeted polypeptide. In some embodiments, the targeted polypeptide is a catalytically inactive nuclease, and the expression cassettes comprise (i) a first expression cassette comprising a first promoter operably linked to a first nucleic acid encoding a gRNA and (ii) a second expression cassette comprising a second promoter operably linked to a second nucleic acid encoding the catalytically-inactive nuclease and the transcriptional activator. In some embodiments, a cell comprises the polynucleotide comprises one or more expression cassettes encoding the targeted polypeptide. In some embodiments, the cell is an adipocyte.

In some embodiments, an engineered adipocyte or adipose stem cell is provided that comprises a targeted polypeptide linked to a transcriptional activator, wherein the targeted polypeptide targets a portion of a promoter or enhancer sequence operably linked to at least one polynucleotide encoding a polypeptide selected from the group consisting of Uncoupling protein 1 (UCP1/SLC25A7), PPARG coactivator 1 alpha (PPARGC1A/PCG1A), PR/SET domain 16 (PRDM16), FFAR1/GPR40, FFAR4/GPR120, FOXO1, GLUT1, GLUT2, GLUT3, GLUT4, CHREBP, CD36, CTP1B, AIFM2/FSP1, GSK, IRS1, DGAT1, DGAT2, UPP1, CKB, and ACACA or as found in Table 2, wherein the targeted polypeptide upregulates expression of the at least one polypeptide compared to a wild-type adipocyte or adipose stem cell.

In some embodiments, an engineered adipocyte or adipose stem cell is provided that comprises: (i) a targeted polypeptide linked to a transcriptional repressor, wherein the targeted polypeptide targets a portion of a promoter or enhancer sequence operably linked to at least one polynucleotide encoding a polypeptide selected from the group consisting of ACACB and NRIP1, wherein the targeted polypeptide downregulates expression of the at least one polypeptide compared to a wild-type adipocyte or adipose stem cell; or (ii) an siRNA or antisense molecule that comprises a sequence complementary to ACACB and NRIP1 and downregulates expression of the at least one polypeptide compared to a wild-type adipocyte or adipose stem cell. In some embodiments, the targeted polypeptide is a catalytically inactive nuclease. In some embodiments, the catalytically inactive nuclease is derived from (i) a clustered regularly interspaced short palindromic repeats (CRISPR) nuclease, and the engineered adipocyte or adipose stem cell further comprises a gRNA that targets the portion of the promoter or enhancer sequence; (ii) a zinc finger nuclease (ZFN); or (iii) a transcription activator-like effector nuclease (TALEN).

In some embodiments, an engineered adipocyte or adipose stem cell is provided that comprises a heterologous expression cassette, wherein the heterologous expression cassette comprises a promoter operably linked to at least one polynucleotide encoding a polypeptide selected from the group consisting of Uncoupling protein 1 (UCP1/SLC25A7), PPARG coactivator 1 alpha (PPARGC1A/PCG1A), PR/SET domain 16 (PRDM16), FFAR1/GPR40, FFAR4/GPR120, FOXO1, GLUT1, GLUT2, GLUT3, GLUT4, CHREBP, CD36, CTP1B, AIFM2/FSP1, GSK, IRS1, DGAT1, DGAT2, UPP1, CKB, and ACACA or as found in Table 2, wherein the heterologous expression cassette overexpresses the at least one polypeptide compared to a wild-type adipocyte or adipose stem cell.

In some embodiments, an engineered adipose organoid is composed of engineered adipocytes or adipose stem cell.

In some embodiments, a method is provided for preventing or treating a metabolic disease or a symptom of aging in a subject. In some embodiments, the method comprises: upregulating or overexpressing at least one polypeptide selected from the group consisting of Uncoupling protein 1 (UCP1/SLC25A7), PPARG coactivator 1 alpha (PPARGC1A/PCG1A), PR/SET domain 16 (PRDM16), FFAR1/GPR40, FFAR4/GPR120, FOXO1, GLUT1, GLUT2, GLUT3, GLUT4, CHREBP, CD36, CTP1B, AIFM2/FSP1, GSK, IRS1, DGAT1, DGAT2, UPP1, CKB, and ACACA or as found in Table 2, or downregulating ACACB or NRIP1, in an adipocyte of the subject, thereby preventing or treating a metabolic disease.

In some embodiments, upregulating the polypeptide comprises introducing to the subject a therapeutically effective amount of a composition comprising a targeted polypeptide linked to a transcriptional activator, wherein the targeted polypeptide targets a portion of a promoter or enhancer sequence operably linked to a polynucleotide encoding the polypeptide. In some embodiments, the targeted polypeptide is a catalytically inactive nuclease, wherein the catalytically inactive nuclease is (i) a clustered regularly interspaced short palindromic repeats (CRISPR) nuclease, and the engineered adipocyte or adipose stem cell further comprises a gRNA that targets the portion of the promoter or enhancer sequence; (ii) a zinc finger nuclease (ZFN); or (iii) a transcription activator-like effector nuclease (TALEN).

In some embodiments, overexpressing the polypeptide comprises introducing to the subject a therapeutically effective amount of a composition comprising a heterologous expression cassette comprising a promoter operably linked to a coding sequence encoding the polypeptide.

In some embodiments, the introducing comprises introducing the composition intravenously, intramuscularly, or a combination thereof.

In some embodiments, downregulating the polypeptide comprises CRISPR interference (CRISPRi), RNA interference (RNAi), or antisense therapy.

In some embodiments, a method for preventing or treating a metabolic disease in a subject is provided that comprises introducing to the subject a therapeutically effective amount of the engineered adipocytes or adipose stem cells or the engineered adipose organoids. In some embodiments, before the introducing, isolating adipocytes or adipose stem cells from the subject and engineering the cells to have the upregulated expression or the overexpression. In some embodiments, the method further comprises culturing the engineered cells into an engineered adipose organoid, and introducing the engineered adipose organoid to the subject. In some embodiments, the introducing engineered adipocytes, or adipose stem cells, or the engineered adipose organoid are autologous. In other embodiments, the introducing engineered adipocytes, or adipose stem cells, or the engineered adipose organoid are allogeneic.

In some embodiments, the introducing comprises introducing the engineered adipocytes, adipose stem cells, or the engineered adipose organoid intravenously, intramuscularly, or a combination thereof. In some embodiments, the subject is a human. In some embodiments, the method improves at least one symptom selected from the group consisting of body weight loss, cold tolerance, oxygen consumption, whole body energy expenditure, glucose and fatty acid utilization, glucose sensitivity, and insulin sensitivity.

In some embodiments, a method for preventing or treating diabetes in a subject is provided that comprises introducing to the subject a therapeutically effective amount of (i) the composition, or (ii) a heterologous expression cassette comprising a promoter operably linked to at least one polynucleotide encoding a polypeptide selected from the group consisting of Uncoupling protein 1 (UCP1/SLC25A7), PPARG coactivator 1 alpha (PPARGC1A/PCG1A), PR/SET domain 16 (PRDM16), FFAR1/GPR40, FFAR4/GPR120, FOXO1, GLUT1, GLUT2, GLUT3, GLUT4, CHREBP, CD36, CTP1B, AIFM2/FSP1, GSK, IRS1, DGAT1, DGAT2, UPP1, CKB, and ACACA or as found in Table 2, or (iii) the engineered adipocytes or adipose stem cells or (iv) the engineered adipose organoid.

In some embodiments, the subject is a human. In some embodiments, the introducing engineered adipocytes, or adipose stem cells, or the engineered adipose organoid are autologous. In other embodiments, the introducing engineered adipocytes, or adipose stem cells, or the engineered adipose organoid are allogeneic. In some embodiments, the introducing comprises introducing the composition, or the heterologous expression cassette, or the engineered adipocytes or adipose stem cells, or the engineered adipose organoid intravenously, intramuscularly, or a combination thereof.

In some embodiments, a method for preventing or treating cancer in a subject is provided that comprises: introducing to the subject a therapeutically effective amount of (i) the composition, or (ii) a heterologous expression cassette comprising a promoter operably linked to at least one polynucleotide encoding a polypeptide selected from the group consisting of Uncoupling protein 1 (UCP1/SLC25A7), PPARG coactivator 1 alpha (PPARGC1A/PCG1A), PR/SET domain 16 (PRDM16), FFAR1/GPR40, FFAR4/GPR120, FOXO1, GLUT1, GLUT2, GLUT3, GLUT4, CHREBP, CD36, CTP1B, AIFM2/FSP1, GSK, IRS1, DGAT1, DGAT2, UPP1, CKB, and ACACA or as found in Table 2, or (iii) the engineered adipocytes or adipose stem cells or (iv) the engineered adipose organoid. In some embodiments, the subject is a human. In some embodiments, the introducing engineered adipocytes, or adipose stem cells, or the engineered adipose organoid are autologous. In other embodiments, the introducing engineered adipocytes, or adipose stem cells, or the engineered adipose organoid are allogeneic.

In some embodiments, the introducing comprises introducing the composition, or the heterologous expression cassette, or the engineered adipocytes or adipose stem cells, or the engineered adipose organoid is intravenously, intramuscularly, or a combination thereof. In some embodiments, the method results in suppression of glycolysis or fatty acid metabolism in cells.

In some embodiments, an adipocyte or adipose stem cell is provided that is engineered to consume a metabolite preferentially consumed by cancer cells compared to non-cancer cells in an individual. In some embodiments, the engineered adipocyte or adipose stem cell is engineered to express uridine phosphorylase (UPP1). In some embodiments, a method of treating or preventing cancer in an individual is provided in which the method comprises introducing into the individual the engineered adipocyte or adipose stem cell as described above or elsewhere herein in proximity to cancer cells that preferentially consume the metabolite engineered adipocyte or adipose stem cells, thereby inhibiting growth of the cancer cells. In some embodiments, the cancer cells are pancreatic ductal adenocarcinoma cells and the engineered adipocyte or adipose stem cells are introduced into the breast of the individual.

In some embodiments, any cell described above or elsewhere herein can be provided in a biocompatible matrix, optionally comprising polycaprolactone. In some embodiments, the cells are delivered into an individual (e.g., a human), for example to achieve a therapeutic effect, and optionally at a later point can be removed from the individual.

### Definitions

Unless specifically indicated otherwise, all technical and scientific terms used herein have the same meaning as commonly understood by those of ordinary skill in the art to which this disclosure belongs. In addition, any method or material similar or equivalent to a method or material described herein can be used in the practice of the present disclosure. For purposes of the present disclosure, the following terms are defined.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “about” as used herein refers to the usual error range for the respective value readily known to the skilled person in this technical field. For example, for K_{D} and IC₅₀ values±20%, ±10%, or ±5%, are within the intended meaning of the recited value.

The words “protein”, “peptide”, and “polypeptide” are used interchangeably to denote an amino acid polymer or a set of two or more interacting or bound amino acid polymers. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refer to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, silent variations of a nucleic acid which encodes a polypeptide is implicit in a described sequence with respect to the expression product, but not with respect to actual probe sequences.

A “conservative” substitution as used herein refers to a substitution of an amino acid such that charge, hydrophobicity, and/or size of the side group chain is maintained. Illustrative sets of amino acids that may be substituted for one another include (i) positively-charged amino acids Lys, Arg and His; (ii) negatively charged amino acids Glu and Asp; (iii) aromatic amino acids Phe, Tyr and Trp; (iv) nitrogen ring amino acids His and Trp; (v) large aliphatic nonpolar amino acids Val, Leu and Ile; (vi) slightly polar amino acids Met and Cys; (vii) small-side chain amino acids Ser, Thr, Asp, Asn, Gly, Ala, Glu, Gln and Pro; (viii) aliphatic amino acids Val, Leu, Ile, Met and Cys; and (ix) small hydroxyl amino acids Ser and Thr. Reference to the charge of an amino acid in this paragraph refers to the charge at physiological pH.

The terms “nucleic acid” and “polynucleotide” are used interchangeably and as used herein refer to both sense and anti-sense strands of RNA, cDNA, genomic DNA, and synthetic forms and mixed polymers of the above. In particular embodiments, a nucleotide refers to a ribonucleotide, deoxynucleotide or a modified form of either type of nucleotide, and combinations thereof. The terms also include, but is not limited to, single- and double-stranded forms of DNA. In addition, a polynucleotide, e.g., a cDNA or mRNA, may include either or both naturally occurring and modified nucleotides linked together by naturally occurring and/or non-naturally occurring nucleotide linkages. Nucleic acid molecules, e.g., oligonucleotide probes or priomers, may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analogue, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). The above term is also intended to include any topological conformation, including single-stranded, double-stranded, partially duplexed, triplex, hairpinned, circular and padlocked conformations. A reference to a nucleic acid sequence encompasses its complement unless otherwise specified. Thus, a reference to a nucleic acid molecule having a particular sequence should be understood to encompass its complementary strand, with its complementary sequence. The term also includes codon-optimized nucleic acids that encode the same polypeptide sequence.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. A “vector” as used here refers to a recombinant construct in which a nucleic acid sequence of interest is inserted into the vector. Certain vectors are capable of directing the expression of nucleic acids to which they are operatively linked. Such vectors are referred to herein as “expression vectors”.

The term “recombinant” or “engineered” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The terms “therapy,” “treatment,” and “amelioration” refer to any reduction in the severity of symptoms. For example, in the case of treating cancer, treatment can refer to reducing the number of cancer cells or growth rate or cell death of non-cancer cells, etc. As used herein, the terms “treat” and “prevent” are not intended to be absolute terms. Treatment and prevention can refer to any delay in onset, amelioration of symptoms, improvement in patient survival, increase in survival time or rate, etc. Treatment and prevention can be complete (no detectable symptoms remaining) or partial, such that symptoms are less frequent of severe than in a patient without the treatment described herein. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

The terms “effective amount,” “effective dose,” “therapeutically effective amount,” etc. refer to that amount of the therapeutic agent sufficient to ameliorate a disorder, as described above. For example, for the given parameter, a therapeutically effective amount will show an increase or decrease of therapeutic effect at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

As used herein, the term “pharmaceutically acceptable” is used synonymously with physiologically acceptable and pharmacologically acceptable. A pharmaceutical composition will generally comprise agents for buffering and preservation in storage, and can include buffers and carriers for appropriate delivery, depending on the route of administration.

The terms “dose” and “dosage” are used interchangeably herein. A dose refers to the amount of active ingredient given to an individual at each administration. The dose will vary depending on a number of factors, including frequency of administration; size and tolerance of the individual; severity of the condition; risk of side effects; the route of administration; and the imaging modality of the detectable moiety (if present). One of skill in the art will recognize that the dose can be modified depending on the above factors or based on therapeutic progress. The term “dosage form” refers to the particular format of the pharmaceutical, and depends on the route of administration. For example, a dosage form can be in a liquid, e.g., a saline solution for injection.

“Subject,” “patient,” “individual” and like terms are used interchangeably and refer to, except where indicated, mammals such as humans and non-human primates, as well as rabbits, rats, mice, goats, pigs, dogs, cats, and other mammalian species. The term does not necessarily indicate that the subject has been diagnosed with a particular disease, but typically refers to an individual under medical supervision. A patient can be an individual that is seeking treatment, monitoring, adjustment or modification of an existing therapeutic regimen, etc.

An “expression cassette” refers to a nucleic acid construct that, when introduced into a host cell, results in transcription and/or translation of an RNA or polypeptide, respectively.

The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “promoter,” as used herein, refers to a polynucleotide sequence capable of driving transcription of a coding sequence in a cell. Thus, promoters can include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription.

The phrase “percent identical” or equivalents used in the context of two nucleic acids or polypeptides, refers to a sequence that has at least a specified level of identity, e.g., at least 50% sequence identity with a reference sequence (e.g., any SEQ ID NO included herein). Alternatively, percent identity can be any integer from 50% to 100%. Some embodiments include at least: 50%, 55%, 60%, 65%, 70%, 75%, 80%, 85%, 90%, 91%, 92%, 93%, 94%, 95%, 96%, 97%, 98%, or 99%, compared to a reference sequence using the programs described herein, e.g., BLAST using standard parameters, as described below.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the numbers of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215:403-410 and Altschul et al. (1977) Nucleic Acids Res. 25:3389-3402, respectively. Software for performing BLAST analyses is publicly available through the National Center for Biotechnology Information (NCBI) web site.

### BRIEF DESCRIPTION OF THE DRAWINGS

FIG. 1A-F. CRISPRa browning activation in human white adipocytes. (A) qRT-PCR of UCP1, PPARGC1A, and PRDM16 in human white adipocytes transduced with CRISPRa targeting UCP1, PPARGC1A, and PRDM16. Data are represented as mean±S.D *≤0.05. (B) qRT-PCR of TFAM, DIO2, CPT1b, and NRF1 in CRISPRa-modulated adipocytes. Data are represented as mean±S.D *≤0.05, ***≤0.001. (C) Oxygen consumption rate (OCR) of CRISPRa-modulated adipocytes measured by the seahorse assay. Uncoupled OCR was measured under oligomycin treatment, while maximal OCR was measured under FCCP. (D) Glucose uptake of CRISPRa-modulated adipocytes with or without insulin. Data are represented as mean±S.D *≤0.05, **≤0.01, ***≤0.001. (E) OCR of CRISPRa-modulated cells was measured by the seahorse assay in BSA- or BSA-Palmitate-medium. Data are represented as mean±S.D *≤0.05, ***≤0.001. (F) Exogenous fatty acid oxidation of CRISPRa-modulated adipocytes was calculated by the difference of area under the curve of OCR between BSA- and BSA-Palmitate-media upon FCCP treatment. Data are represented as mean±S.D *≤0.05, **≤0.01.

FIG. 2A-K. CRISPRa-modulated adipocytes inhibit cancer cell growth in vitro. (A) Schematic of the co-culturing model of cancer cells and CRISPRa-treated adipocytes using transwell plates and their subsequent phenotyping. (B) Representative images of cancer cells, including breast (MCF7, MDA-MD-436), colon (SW-1417), pancreatic (Panc 10.05), and prostate cancer (DU-145) that were co-cultured with CRISPRa upregulating UCP1, PPARGC1a, and PRDM16 or control (dCas9-VP64 only) adipocytes. (C) Cancer cell numbers per view of image (4 images/replicates per condition). Data are represented as mean±S.D ***≤0.001. (D) qRT-PCR of the proliferation marker gene, MKI67, for cancer cells co-cultured with CRISPRa-modulated adipocytes. Data are represented as mean±S.D *≤0.05, **≤0.01, ***≤0.001. (E) Basal glycolysis measured by calculating area under the curve of extracellular acidification rate upon glucose treatment. Data are represented as mean±S.D *≤0.05, **≤0.01, ***≤0.001. (F) Maximal glycolysis measured by calculating area under the curve of extracellular acidification rate upon oligomycin treatment. Data are represented as mean±S.D *≤0.05, **≤0.01, ***≤0.001. (G-H) Glucose uptake of cancer cells co-cultured with CRISPRa-modulated adipocytes without (G) or with (H) insulin. Data are represented as mean±S.D *≤0.05, **≤0.01, ***≤0.001. (I) qRT-PCR of glucose transporter, GLUT4, and glycolytic enzyme, GCK in cancer cells. Data are represented as mean±S.D *≤0.05, **≤0.01, ***≤0.001. (J) Exogenous fatty acid oxidation of cancer cells calculated by the difference of area under the curve of OCR between BSA- and BSA-Palmitate-media upon FCCP treatment. Data are represented as mean±S.D **≤0.01, ***≤0.001. (K) qRT-PCR of fatty acid transporter, CD36, and fatty acid regulatory transporter, CPT1b in cancer cells that were co-cultured with CRISPRa-treated adipocytes. Data are represented as mean±S.D *≤0.05, ***≤0.001.

FIG. 3A-F. Co-transplantation of xenografts with UCP1-CRISPRa modulated human adipose organoids suppresses tumor growth. (A) Schematic of the co-transplantation model for xenografts and CRISPRa-UCP1 treated human adipose organoids in immune-deficient SCID mice and their subsequent phenotyping. (B) Representative images of xenograft tumors from various cancer cells lines, including breast (MCF7 and MDA-MD-436), colon (SW-1417), pancreatic (Panc 10.05), and prostate cancer (DU-145) that were co-transplanted with UCP1-CRISPRa human adipose organoids or control (dCas9-VP64 only) adipose organoids (n=8 mice per treatment). (C) Volume of xenograft tumors that were co-transplanted with CRISPRa-modulated human adipose organoids compared to control (dCas9-VP64 only) (n=6-8 mice). Data are represented as mean±S.D ***≤0.001. (D-E) Immunofluorescence staining and quantification of Ki67 (D), CA9 (E), and CD31 (F) in cryosections of xenograft tumors (n=4 sections per treatment). Data are represented as mean±S.D ***≤0.001.

FIG. 4A-L. Implantation of Ucp1-CRISPRa adipose organoids in pancreatic and breast cancer genetic mouse models suppresses cancer development. (A) Schematic of the transplantation model for Ucp1-CRISPRa treated mouse adipose organoids in KPC pancreatic cancer mice and their subsequent phenotyping. (B) Representative images of the pancreas implanted with Ucp1-CRISPRa or control (dCas9-VP64 only) mouse adipose organoids (n=5-6 mice per treatment). (C) Weight of the pancreas transplanted with Ucp1-CRISPRa modulated mouse adipose organoids compared to control (dCas9-VP64 only) (n=5-6 mice). Data are represented as mean±S.D **≤0.01. (D) qRT-PCR of proliferation marker gene, MKI67, metabolic genes, including Glut2, Gck, Cd36, and Ctplb, from pancreatic tumors co-transplanted with Ucp1-CRISPRa modulated adipocytes. Data are represented as mean±S.D *≤0.05, **≤0.01. (E) Immunofluorescence quantification of Ki67, CA9, and CD31 in cryosections of tumors (n=4 sections per treatment). Data are represented as mean±S.D *≤0.05, **≤0.01. (F) Schematic of the transplantation model for Ucp1-CRISPRa treated mouse adipose organoids in the mammary gland or on the back of MMTV-PyMT breast cancer mice and their subsequent phenotyping. (G) Representative images of the breast tumor that were implanted with Ucp1-CRISPRa or control (dCas9-VP64 only) adipose organoids in the mammary gland or on the back of the mice (Dorsal) (n=4-5 mice per treatment). (H) Volume of the tumors transplanted with Ucp1-CRISPRa adipose organoids compared to control (dCas9-VP64 only) (n=4-5 mice). Data are represented as mean±S.D *≤0.05, **≤0.01. (I) qRT-PCR of proliferation marker gene, MKI67, metabolic genes, including GLUT4, GCK, CD36, and CPT1b, from breast tumors co-transplanted with Ucp1-CRISPRa modulated adipocytes. Data are represented as mean #S.D *≤0.05, **≤0.01, ***≤0.001. (J-L) Immunofluorescence staining and quantification of Ki67 (J), CA9 (K), and CD31 (L) in tumor cryosections (n=4 sections per treatment). Data are represented as mean±S.D **≤0.01, ***≤0.001.

FIG. 5A-E. Cancer organoids co-cultured with UCP1-CRISPRa, both from dissected breast tissue, lead to tumor suppression. (A) Schematic of the co-culturing model of UCP1-CRISPRa modulated human mammary adipocytes and breast cancer organoids from dissected breast tumors. (B) Representative images of breast tumor organoids from five dissected breast tumors that were co-cultured with UCP1-CRISPRa adipocytes or control (dCas9-VP64 only) adipocytes. (C) Breast cancer organoid size and numbers. Data are represented as mean±S.D *≤0.05, **≤0.01, ***≤0.001. (D) qRT-PCR of proliferation marker gene, MKI67. Data are represented as mean±S.D *≤0.05, **≤0.01. (E) qRT-PCR of metabolic genes, including GLUT4, GCK, CD36, and CPT1b of breast cancer organoids that were co-cultured with CRISPRa-modulated adipocytes. Data are represented as mean #S.D *≤0.05, **≤0.01, ***≤0.001.

FIG. 6A-J. CRISPRa modulating Ucp1, Ppargc1a, and Prdm16 reduces adiposity in mice. (A) Schematic of AAV-CRISPRa targeting Ucp1, Ppargc1a, and Prdm16 in C57Bl/6J mice on chow diet. (B) Representative images and body weight of mice treated with AAV-CRISPRa or dCas-VP64 only (control). (C) Fat mass and lean mass measured by DEXA (dual energy X-ray absorptiometry). (D) Representative images and mass of fat depots, including BAT, iWAT, and pWAT, and kidney. (E) RT-qPCR of mCherry, Ucp1, Ppargc1a, and Prdm16 in adipose tissue, liver, and muscle. (F) Core body temperature measured via rectal of mice kept at 4° C. (G) Whole-body oxygen consumption measured my CLAMS (Comprehensive Lab Animal Monitoring System). (H) Glucose tolerance test performed after overnight fasting. (I) Insulin tolerance test. (J) Plasma insulin measured by ELISA. Data are represented as mean±S.D *≤0.05, **≤0.01, ***≤0.001.

FIG. 7A-F. CRISPRa modulating Ucp1, Ppargc1a, and Prdm16 protects mice against high fat diet-induced obesity. (A) Schematic of AAV-CRISPRa targeting Ucp1, Ppargc1a, and Prdm16 in C57Bl/6J mice on high fat diet (HFD). Body weight (B) and representative images (C) of mice treated with AAV-CRISPRa or dCas-VP64 (control). (D) Fat mass and lean mass measured by DEXA and representative images of fat depots, including BAT, iWAT, and pWAT, and kidney. (E) Insulin tolerance test performed after overnight fasting. (F) Insulin tolerance test. Data are represented as mean±S.D *≤0.05, **≤0.01, *≤0.001.

FIG. 8A-J. CRISPRa modulating Ucp1, Ppargc1a, and Prdm16 reduces weight gain and improves insulin response in ob/ob mice. Schematic of AAV-CRISPRa targeting Ucp1, Ppargc1a, and Prdm16 injecting in ob/ob mice treated at four weeks (A) or 10 weeks (B). Body weight of ob/ob mice treated at four weeks (C) or 10 weeks (D). (E) Representative images of ob/ob mice treated with AAV-CRISPRa at four weeks (left) or 10 weeks (right). (F) Representative images of fat depots, including BAT, iWAT, and pWAT and fat mass and lean mass measured by DEXA of mice treated with AAV-CRISPRa at four weeks (left) or 10 weeks (right). (G) Insulin tolerance test and (H) insulin tolerance test of ob/ob mice treated with AAV-CRISPRa at four weeks (left) or 10 weeks (right). (I) Plasma insulin and (J) free fatty acid of ob/ob mice treated at four weeks. Data are represented as mean±S.D *≤0.05, **≤0.01, ***≤0.001.

FIG. 9A-K. Transplantation of CRISPRa modulated mouse adipose tissue or human adipose organoids protects mice against obesity. (A) Schematic showing the transplantation of iWAT of mice treated with AAV-CRISPRa targeting Ucp1, Ppargc1a, and Prdm16 in C57BL/6J mice. (B) Body weight of mice transplanted with fat of mice treated with AAV-CRISPRa or dCas-VP64 (control). (C) Glucose tolerance test. (D) Insulin tolerance test. (E) Plasma insulin and free fatty acid levels. (F) Schematic showing the transplantation of human adipose organoids treated with AAV-CRISPRa targeting UCP1, PPARGC1a, and PRMD16 in SCID mice. (G) Body weight of mice transplanted with human adipose organoids treated with AAV-CRISPRa or dCas-VP64 (control). (H) Representative images and fat mass of SCID mice implanted with CRISPRa treated human adipose organoids. (I) Representative images and weight of fat depots, including BAT, iWAT, and pWAT, and kidney. (J) Glucose tolerance test. (K) Insulin tolerance test. Data are represented as mean±S.D *≤0.05, **≤0.01, ***≤0.001.

FIG. 10A-F. CRISPRa modulating Foxo1, Ffar1, and Ffar4 improves glucose clearance and insulin sensitivity in mice. RT-qPCR of Foxo1, Ffar1, and Ffar4 in 3T3-L1 adipocytes transfected (A) or infected with AAV-CRISPRa targeting Foxo1, Ffar1, and Ffar4. (B) Representative images and body weight of mice treated with AAV-CRISPRa or dCas-VP64 (control). (C) Glucose tolerance test performed after overnight fasting and (I) insulin tolerance test of mice injected with AAV-CRISPRa targeting Foxo1, Ffar1, and Ffar4 and fed with chow diet. (E) Glucose tolerance test and (F) insulin tolerance test of mice injected with AAV-CRISPRa targeting Foxo1, Ffar1, and Ffar4 and fed with high fat diet. Data are represented as mean±S.D *≤0.05, **≤0.01.

FIG. 11A-B. CRISPRa upregulation of UCP1, PPARGC1A, and PRDM16 in human white adipocytes. (A) qRT-PCR of UCP1, PPARGC1A, and PRDM16 in white adipocytes transfected with CRISPRa using five different gRNAs per gene. Data are represented as mean±S.D. *≤0.05, **≤0.01, ***≤0.001. (B) qRT-PCR of UCP1, PPARGC1A, and PRDM16 in human adipocytes transduced by AAV9 CRISPRa with the top two gRNAs per gene. Data are represented as mean±S.D. *≤0.05, **≤0.01, ***≤0.001.

FIG. 12A-C. Proliferation and metabolic rate of various cancer cells co-cultured with CRISPRa-modulated human adipocytes. (A) BrdU incorporation, (B) Oxygen consumption rate (OCR), and (C) Fatty acid oxidation of various cancer cells (MCF-7, MDA-MB-436, SW-1417, Panc.10.05, and Du-145) that were co-culture with CRISPRa-modulated adipocytes. Data are represented as mean±S.D. *≤0.05, **≤0.01, ***≤0.001.

FIG. 13A-G. CRISPRa upregulation of UCP1, PPARGC1A, and PRDM16 in human adipose organoids and proliferation and metabolic gene expression in tumors co-transplanted with CRISPRa-modulated adipose organoids. (A) Representative images of human adipose organoids transduced with UCP1, PPARGC1A, and PRDM16 AAV9 CRISPRa showing mCherry expression. (B) qRT-PCR of FABP4, PLIN1, and ADIPOQ in adipose organoids. Data are represented as mean±S.D. *≤0.05, **≤0.01, ***≤0.001. (C) qRT-PCR of UCP1, PPARGC1A, and PRDM16 in human adipose organoids transduced by AAV9 CRISPRa. Data are represented as mean±S.D. **≤0.01, ***≤0.001. (D) Body weight of mice that were co-transplanted with UCP1-modulated adipose organoids and cancer cells. (E) qRT-PCR of MKI67 in xenograft tumors derived from MCF-7, MDA-MB-436, Panc 10.05, and DU-145 cancer cells that were co-transplanted with UCP1-CRISPRa modulated adipose organoids. Data are represented as mean±S.D. **≤0.01, ***≤0.001. (F) qRT-PCR of GLUT4, GCK, CD36, and CTP1B in xenograft tumors. Data are represented as mean±S.D. *≤0.05, **≤0.01, ***≤0.001. (G) Plasma insulin levels of wild type (WT) SCID mice and mice that were co-transplanted with UCP1-CRISPRa adipose organoids and cancer cells. Data are represented as mean±S.D. **≤0.01, ***≤0.001.

FIG. 14A-H. CRISPRa upregulation of Ucp1 in mouse white adipocytes and phenotypic analysis of mouse genetic cancer models. (A) qRT-PCR of Ucp1, in white adipocytes transfected with CRISPRa using five different gRNAs. Data are represented as mean±S.D. *≤0.05, ***≤0.001. (B) qRT-PCR of Ucp1 in mouse adipocytes transduced by AAV9-CRISPRa with the top two gRNAs per gene. Data are represented as mean±S.D. **≤0.01. (C) qRT-PCR of mcherry and Ucp1 in mouse adipose organoids. Data are represented as mean±S.D. ***≤0.001. (D) Body weight of pancreatic cancer KPC mice implanted with control (dCas9-VP64) Ucp1-upregulated mouse adipose organoids. (E) Immunofluorescence staining and quantification of Ki67, CA9, and CD31 in cryosections of pancreatic tumors. (F) Plasma insulin levels of pancreatic cancer genetic mice implanted with either Ucp1-CRISPRa adipose organoids or control organoids. Data are represented as mean±S.D. *≤0.05. (G) Body weight of breast cancer (MMTV-PyMT) mice implanted with control (dCas9-VP64) or Ucp1-CRISPRa mouse adipose organoids nearby mammary gland (MG) or distal (DOR). (H) Plasma insulin levels of breast cancer genetic mice implanted with either Ucp1-CRISPRa adipose organoids or control organoids nearby mammary gland (MG) or distal (DOR). Data are represented as mean±S.D. *≤0.05.

FIG. 15A-B. CRISPRa upregulation of UCP1 in human mammary gland adipocytes. (A) Representative images of adipocytes isolated from mammary glands that were transduced with UCP1-CRISPRa AAV9. (B) qRT-PCR of dCas9, mcherry, and UCP1 in CRISPRa-modulated mammary gland adipocytes. Data are represented as mean±S.D. *≤0.05, **≤0.01, ***≤0.001.

FIG. 16A-B depict results showing UCP1-overexpressing human adipocytes suppress MCF7 cancer cell growth. (A) Representative images of MCF7 breast cancer cells that were co-cultured with UCP1-overexpressing human adipocytes. (B) qRT-PCR of MKI67, a proliferative marker, and metabolic genes, including GLUT4, GCK, CD36, and CTP1b.

### DETAILED DESCRIPTION OF THE INVENTION

The present disclosure is directed to compositions and methods to prevent and treat metabolic diseases, diabetes, and cancers through regulating expression of the genes associated with glucose and lipid metabolism. The inventors have found that upregulating or overexpressing certain gene products or downregulating other gene products in adipocytes and other cells can convert these cells to a brown fat phenotype with increased cell glucose and lipid metabolism. Implantation of these cells results in reduction of obesity, reduction of metabolic diseases (including but not limited to type II diabetes) and also reduction of cancers. As explained further herein, one or more of the gene products can be upregulated using a targeted polypeptide linked to a transcriptional activator to increase expression of the gene products in a cell from the native gene sequence or by overexpression of the gene products, for example by introduction of one or more heterologous expression cassettes encoding the gene products. In addition, one or more of the gene products can be downregulated using a targeted polypeptide linked to a transcriptional repressor to decrease expression of the gene products or otherwise downregulate expression of the gene products (e.g., using siRNAs, antisense or other down regulation methods). Optionally, upregulation/overexpression of one or more gene products can be combined with downregulation of one or more other gene products in the same cells, to facilitate converting to a brown fat phenotype.

### Genes of Interest

Any of the following gene products can be upregulated or over expressed in mesenchymal stem cells, pre-adipocytes, fat cells, adipocytes, adipose organoids or fat progenitor cells as described herein to make cells having the desired metabolic phenotype.

TABLE 1
Representative genes and proteins associated with glucose
and lipid metabolism, and promoter information.

| Gene | Gene | | | |
|---|---|---|---|---|
| name | ID | Protein name | UniProt | Promoter sequence (hg38) |
| UCP1 | 7350 | UCP1/SLC25A7 | P25874 | chr4: 140566961-140570961 |
| | | (SEQ ID NO: 57) | | (SEQ ID NO: 78) |
| PPARGC1A | 10891 | PPARGC1A/PCG1A | Q9UBK2 | chr4: 23888047-23892047 |
| | | (SEQ ID NO: 58) | | (SEQ ID NO: 79) |
| | | | | chr4: 23,874,636-23,888,871 |
| | | | | (SEQ ID NO: 80) |
| PRDM16 | 63976 | PRDM16 | Q9HAZ2 | chr1: 3067202-3071202 |
| | | (SEQ ID NO: 59) | | (SEQ ID NO: 81) |
| | | | | chr1: 3066709-3070709 |
| | | | | (SEQ ID NO: 82) |
| FFAR1 | 2864 | FFAR1/GPR40 | O14842 | chr19: 35,345,385-35,348,325 |
| | | (SEQ ID NO: 60) | | (SEQ ID NO: 83) |
| FFAR4 | 338557 | FFAR4/GPR120 | Q5NUL3 | chr10: 93564664-93568664 |
| | | (SEQ ID NO: 61) | | (SEQ ID NO: 84) |
| | | | | chr10: 93552640-93556640 |
| | | | | (SEQ ID NO: 85) |
| FOXO1 | 2308 | FOXO1 | Q12778 | chr13: 40664588-40668588 |
| | | (SEQ ID NO: 62) | | (SEQ ID NO: 86) |
| | | | | chr13: 40665050-40669050 |
| | | | | (SEQ ID NO: 87) |
| SLC2A1 | 6513 | GLUT1 | P11166 | chr1: 42956868-42960868 |
| | | (SEQ ID NO: 63) | | (SEQ ID NO: 88) |
| SLC2A2 | 6517 | GLUT2 | P14672 | chr3: 171024720-171028720 |
| | | (SEQ ID NO: 64) | | (SEQ ID NO: 89) |
| SLC2A3 | 6517 | GLUT3 | P11169 | chr12: 7934187-7938187 |
| | | (SEQ ID NO: 65) | | (SEQ ID NO: 90) |
| SLC2A4 | 6517 | GLUT4 | P14672 | chr17: 7279717-7283717 |
| | | (SEQ ID NO: 66) | | (SEQ ID NO: 91) |
| MLXIPL | 51085 | CHREBP | Q9NP71 | chr7: 73622522-73626522 |
| | | (SEQ ID NO: 67) | | (SEQ ID NO: 82) |
| CD36 | 948 | CD36 | P16671 | chr7: 80636641-80640641 |
| | | (SEQ ID NO: 68) | | (SEQ ID NO: 93) |
| | | | | chr7: 80600206-80604206 |
| | | | | (SEQ ID NO: 94) |
| CPT1B | 1375 | CTP1B | Q92523 | chr22: 50576409-50580409 |
| | | (SEQ ID NO: 69) | | (SEQ ID NO: 95) |
| | | | | chr22: 50566972-50570972 |
| | | | | (SEQ ID NO: 96) |
| AIFM2 | 84883 | AIFM2/FSP1 | Q9BRQ8 | chr10: 70168512-70172512 |
| | | (SEQ ID NO: 70) | | (SEQ ID NO: 97) |
| | | | | chr10: 70,123,400-70,137,856 |
| | | | | (SEQ ID NO: 98) |
| GSK3B | 605004 | GSK | P49841 | chr3: 120092447-120096447 |
| | | (SEQ ID NO: 71) | | (SEQ ID NO: 99) |
| | | | | chr3: 120091696-120095696 |
| | | | | (SEQ ID NO: 100) |
| | | | | chr3: 120092296-120096296 |
| | | | | (SEQ ID NO: 101) |
| IRS1 | 3667 | IRS1 | P35568 | chr2: 226797813-226801813 |
| | | (SEQ ID NO: 72) | | (SEQ ID NO: 102) |
| | | | | chr2: 226797179-226801179 |
| | | | | (SEQ ID NO: 103) |
| DGAT1 | 8694 | DGAT1 | O75907 | chr8: 144324852-144328852 |
| | | (SEQ ID NO: 73) | | (SEQ ID NO: 104) |
| DGAT2 | 84649 | DGAT2 | Q96PD7 | chr11: 75766756-75770756 |
| | | (SEQ ID NO: 74) | | (SEQ ID NO: 105) |
| | | | | chr11: 75766950-75770950 |
| | | | | (SEQ ID NO: 106) |
| ACACA | 31 | ACACA | Q13085 | chr17: 37357096-37361096 |
| | | (SEQ ID NO: 75) | | (SEQ ID NO: 107) |
| | | | | chr17: 37404833-37408833 |
| | | | | (SEQ ID NO: 108) |
| ACACB | 32 | ACACB | O00763 | chr12: 109129360-109133360 |
| | | (SEQ ID NO: 76) | | (SEQ ID NO: 109) |
| | | | | chr12: 109114586-109118586 |
| | | | | (SEQ ID NO: 110) |
| | | | | chr12: 109111288-109115288 |
| | | | | (SEQ ID NO: 111) |
| | | | | chr12: 109,109,388-109,117,067 |
| | | | | (SEQ ID NO: 112) |
| NRIP1 | 8204 | NRIP1 | P48552 | chr21: 15062988-15066988 |
| | | (SEQ ID NO: 77) | | (SEQ ID NO: 113) |
| | | | | chr21: 15063936-15067936 |
| | | | | (SEQ ID NO: 114) |
| UPP1 | | UPP1 (SEQ ID | | SEQ ID NO:116 |
| | | NO: 115) | | |
| CKB | 1152 | CKB (SEQ ID | P12277 | chr14: 103,521,980-103,523,794 |
| | | NO: 117) | | (SEQ ID NO: 118): |

Uncoupling protein 1 (UCP1, or SLC25A7) is responsible for thermogenic respiration and generally to the regulation of energy balance. Our study showed that upregulation of UCP1 reduces body weight, fat mass, improves insulin sensitivity, and inhibits cancer growth.

PPARG coactivator 1 alpha (PPARGC1A, or PCG1A) is a transcriptional coactivator for steroid receptors and nuclear receptors. Greatly increases the transcriptional activity of PPARG and thyroid hormone receptor on the uncoupling protein promoter. Our study shows that upregulation of PPARGC1A reduces body weight, fat mass, improves insulin sensitivity, and inhibits cancer growth.

PR/SET domain 16 (PRDM16) binds DNA and functions as a transcriptional regulator. Functions in the differentiation of brown adipose tissue (BAT) which is specialized in dissipating chemical energy in the form of heat in response to cold or excess feeding while white adipose tissue (WAT) is specialized in the storage of excess energy and the control of systemic metabolism. Our study shows that upregulation of PRDM16 reduces body weight, fat mass, improves insulin sensitivity, and inhibits cancer growth.

FFAR1 or GPR40 is a G-protein coupled receptor for medium and long chain saturated and unsaturated fatty acids that plays an important role in glucose homeostasis. Our study shows that upregulation of FFA1R improves insulin sensitivity.

FFAR4 or GPR120 is a G-protein-coupled receptor for long-chain fatty acids (LCFAs) with a major role in adipogenesis, energy metabolism and inflammation. Our study show upregulation of FFA4R improves insulin sensitivity.

FOXO1 is a transcription factor that is the main target of insulin signaling and regulates metabolic homeostasis in response to oxidative stress. Our study show upregulation of FOXO1 improves insulin sensitivity.

GLUT1 is a facilitative glucose transporter, which is responsible for constitutive or basal glucose uptake. Upregulation of SLC2A1 can improves insulin sensitivity and inhibits cancer growth.

GLUT2 is a facilitative hexose transporter that mediates the transport of glucose, fructose and galactose. Upregulation of SLC2A2 can improves insulin sensitivity and inhibits cancer growth.

GLUT3 is a facilitative glucose transporter that can also mediate the uptake of various other monosaccharides across the cell membrane. Upregulation of SLC2A3 can improves insulin sensitivity and inhibits cancer growth.

GLUT4 is an Insulin-regulated facilitative glucose transporter, which plays a key role in removal of glucose from circulation. Upregulation of SLC2A4 can improves insulin sensitivity and inhibits cancer growth.

CHREBP is a glucose sensing transcription factor. Upregulation of MLXIPL can improves insulin sensitivity and inhibits cancer growth.

CD36 is a multifunctional glycoprotein that acts as a receptor for a broad range of ligands, including long-chain fatty acids. Upregulation of CD36 can reduce body weight, improves insulin sensitivity and inhibits cancer growth.

CPT1B is a member of the carnitine/choline acetyltransferase family, is the rate-controlling enzyme of the long-chain fatty acid beta-oxidation pathway. Upregulation of CTP1B can reduce body weight, improves insulin sensitivity and inhibits cancer growth.

AIFM2 or FSB1 has functions in the glycolysis of brown adipose tissue (BAT). Upregulation of AIFM2 can reduce body weight, fat mass, improves insulin sensitivity, and inhibits cancer growth.

GSK is a constitutively active protein kinase that acts as a negative regulator in the hormonal control of glucose homeostasis. Upregulation of GSK3B can reduce body weight, fat mass, improves insulin sensitivity, and inhibits cancer growth.

IRS1 mediates the control of various cellular processes by insulin. Upregulation of IRS1 can reduce body weight, fat mass, improves insulin sensitivity, and inhibits cancer growth.

DGAT1 and DGAT2 are fatty acid breakdown enzymes.

ACACA and ACACB are biotin-containing enzymes that catalyze the carboxylation of acetyl-CoA to malonyl-CoA, which is the rate-limiting step in fatty acid synthesis.

NRIP1 is a transcriptional co-repressor that reduces the activity of various metabolic nuclear receptors in adipocytes by interacting with their AF2 domain. These lead to the reduction of UCP1, NRG4 and ESRRA expression and suppression of mitochondrial respiration, glucose transport and fatty acid oxidation.

CKB, creatine kinase B, is a cytoplasmic enzyme involved in energy homeostasis. It transfers phosphate between ATP and phsophgens.

### Upregulating Gene Expression

Compositions and methods for preventing or treating metabolic diseases, diabetes, and cancers in a subject are provided. In some embodiments, the compositions and methods upregulate expression of at least one gene associated with glucose and lipid metabolism, i.e., Uncoupling protein 1 (UCP1/SLC25A7), PPARG coactivator 1 alpha (PPARGC1A/PCG1A), PR/SET domain 16 (PRDM16), FFAR1/GPR40, FFAR4/GPR120, FOXO1, GLUT1, GLUT2, GLUT3, GLUT4, CHREBP, CD36, CTP1B, AIFM2/FSP1, GSK, IRS1, DGAT1, DGAT2, UPP1, CKB and/or ACACA or as found in Table 2, in one or more cells (e.g., in mesenchymal stem cells, pre-adipocytes, fat cells, adipocytes, adipose organoids or fat progenitor cells) in the subject. Upregulating the expression of the gene of interest can include, for example, introducing a targeted polypeptide linked to a transcriptional activator into the cell, wherein the targeted polypeptide targets a portion of a promoter or enhancer sequence operably linked to the gene of interest, thereby increasing expression of the gene product. A targeted polypeptide refers to a polypeptide that specifically targets a promoter or enhancer sequence either as a function of the polypeptide's direct affinity for the promoter or enhancer sequence or as guided by a nucleic acid sequence (for example as a gRNA-guided CRISPR nuclease). For example, catalytically-inactive Cas protein variants may be fused to transcriptional activation domains to control gene expression (Ma et al., Protein and Cell, 2(11):879-888, 2011; Maeder et al., Nature Methods, 10:977-979, 2013; and Konermann et al., Nature, 517:583-588, 2014).

The targeted polypeptide is covalently or non-covalently linked to a transcriptional activator that upregulates the expression of the gene of interest. The targeted polypeptide targets a portion of a promoter or enhancer sequence operably linked to the gene of interest and thus brings the transcriptional activator close to the promoter or enhancer to enhance transcription of the gene of interest.

In some embodiments, the targeted polypeptide is an inactive nuclease. Examples of nucleases include, but are not limited to, a zinc finger nuclease (ZFN), a transcription activator-like effector nuclease (TALEN), a clustered regularly interspaced short palindromic repeats (CRISPR) nuclease, a site-specific recombinase, a transposase, a topoisomerase, and modified derivatives and variants thereof. In some embodiments, the nuclease is capable of targeting a designated nucleotide or region within the target gene. In some embodiments, the nuclease is capable of targeting a region positioned between the 5′ and 3′ regions of the target gene. In another embodiment, the nuclease is capable of targeting a region positioned upstream or downstream of the 5′ and 3′ regions of the target gene (e.g., upstream or downstream of the transcription start site (TSS)). A recognition sequence is a polynucleotide sequence that is specifically recognized and/or bound by the targeted polypeptide. The length of the recognition site sequence can vary, and includes, for example, nucleotide sequences that are at least 10, 12, 14, 16, 18, 19, 20, 21, 22, 23, 24, 25, 26, 27, 28, 29, 30, 35, 40, 45, 50, 55, 60, 65, 70 or more nucleotides in length. Inactivated nucleases are polypeptides that retain the DNA-binding ability of a nuclease while substantially lacking cleaving activity. In some embodiments, the nuclease can be identical to a native of modified nuclease but having 1, 2, 3, 4 or more amino acid changes that inactivate the nuclease (cleaving) activity.

In some embodiments, the targeted polypeptide is a catalytically inactive nuclease. In certain aspects, the catalytically inactive nuclease is derived from a zinc finger nuclease (ZFN), a transcription activator-like effector nuclease (TALEN), a clustered regularly interspaced short palindromic repeats (CRISPR) nuclease, a site-specific recombinase, a transposase, a topoisomerase, and modified derivatives and variants thereof. In some embodiments, the targeted polypeptide is a catalytically inactive ZFN. In some embodiments, the targeted polypeptide is a catalytically inactive TALEN. In some embodiments, the targeted polypeptide is a TAL effector (TALE). In some embodiments, the targeted polypeptide is a catalytically inactive clustered regularly interspaced short palindromic repeats (CRISPR) nuclease. In certain embodiments, the targeted polypeptide is dCas9.

### Zinc Finger Nuclease (ZFN)

In some embodiments, the targeted polypeptide is a zinc-finger nuclease (ZFN) or a modified derivative. ZFNs typically comprise a zinc finger DNA binding domain and a nuclease domain. Generally, ZFNs include two zinc finger arrays (ZFAs), each of which is fused to a single subunit of a non-specific endonuclease, such as the nuclease domain from the FokI enzyme, which becomes active upon dimerization. Typically, a single ZFA consists of 3 or 4 zinc finger domains, each of which is designed to recognize a specific nucleotide triplet (GGC, GAT, etc.). A ZFN composed of two “3-finger” ZFAs is therefore capable of recognizing an 18 base pair target site (i.e., recognition sequence); an 18 base pair recognition sequence is generally unique, even within large genomes such as those of humans and plants. By directing the co-localization and dimerization of the two FokI nuclease monomers, ZFNs generate a functional site-specific endonuclease that can target a particular locus (e.g., gene, promoter or enhancer).

Zinc-finger nucleases useful in the methods disclosed herein include those that are known and ZFN that are engineered to have specificity for one or more target sites described herein (e.g., promotor or enhancer nucleotide sequence). Zinc finger domains are amenable for designing polypeptides which specifically bind a selected polynucleotide recognition sequence within a target site of the host cell genome. ZFN can comprise an engineered DNA-binding zinc finger domain linked to a non-specific endonuclease domain, for example, a nuclease domain from a Type IIs endonuclease such as HO or FokI. In some examples, a zinc finger DNA binding domain can be fused to a site-specific recombinase, transposase, or a derivative thereof that retains DNA nicking and/or cleaving activity.

Each zinc finger domain recognizes three consecutive base pairs in the target DNA. For example, a three-finger domain recognizes a sequence of nine contiguous nucleotides, with a dimerization requirement of the nuclease, two sets of zinc finger triplets are used to bind a 18 nucleotide recognition sequence. Useful zinc finger modules include those that recognize various GNN and ANN triplets (Dreier et al., (2001) J Biol Chem 276:29466-78; Dreier et al., (2000) J Mol Biol 303:489-502; Liu et al., (2002) J Biol Chem 277:3850-6), as well as those that recognize various CNN or TNN triplets (Dreier et al., (2005) J Biol Chem 280:35588-97; Jamieson et al., (2003) Nature Rev Drug Discovery 2:361-8). See also, Durai et al., (2005) Nucleic Acids Res 33:5978-90; Segal, (2002) Methods 26:76-83; Porteus and Carroll, (2005) Nat Biotechnology 23:967-73; Pabo et al., (2001) Ann Rev Biochem 70:313-40; Wolfe et al., (2000) Ann Rev Biophys Biomol Struct 29:183-212; Segal and Barbas (2001) Curr Opin Biotechnol 12:632-7; Segal et al., (2003) Biochemistry 42:2137-48; Beerli and Barbas, (2002) Nat Biotechnol 20:135-41; Carroll et al., (2006) Nature Protocols 1:1329; Ordiz et al., (2002) Proc Natl Acad Sci USA 99:13290-5; Guan et al., (2002) Proc Natl Acad Sci USA 99:13296-301; WO2002099084; WO00/42219; WO02/42459; WO2003062455; US20030059767; US Pat. Application Publication Number 2003/0108880; U.S. Pat. Nos. 6,140,466, 6,511,808 and 6,453,242. Useful zinc-finger nucleases also include those described in WO03/080809; WO05/014791; WO05/084190; WO08/021207; WO09/042186; WO09/054985; and WO10/065123.

### TALENs

In some embodiments of the methods and compositions described herein, the targeted polypeptide is derived from a transcription activator-like effector nuclease (TALEN). In some embodiments, the targeted polypeptide is a transcription activator-like effector (TALE). TAL effectors (TALEs) are proteins secreted by Xanthomonas bacteria and play an important role in disease or triggering defense mechanisms, by binding host DNA and activating effector-specific host genes. see, e.g., Gu et al. (2005) Nature 435:1122-5; Yang et al., (2006) Proc. Natl. Acad. Sci. USA 103:10503-8; Kay et al., (2007) Science 318:648-51; Sugio et al., (2007) Proc. Natl. Acad. Sci. USA 104:10720-5; Romer et al., (2007) Science 318:645-8; Boch et al., (2009) Science 326(5959):1509-12; and Moscou and Bogdanove, (2009) 326(5959):1501. A TALEN comprises a TAL effector DNA-binding domain fused to a DNA cleavage domain, and in embodiments herein, the cleavage domain can include one or more mutations such that the cleavage domain is inactive. The DNA binding domain interacts with DNA in a sequence-specific manner through one or more tandem repeat domains. The repeated sequence typically comprises 33-34 highly conserved amino acids with divergent 12th and 13th amino acids. These two positions, referred to as the Repeat Variable Diresidue (RVD) are highly variable and show a strong correlation with specific nucleotide recognition (Boch et al., (2009) Science 326(5959):1509-12; and Moscou and Bogdanove, (2009) 326(5959):1501). This relationship between amino acid sequence and DNA recognition sequence has allowed for the engineering of specific DNA-binding domains by selecting a combination of repeat segments containing the appropriate RVDs.

The TAL-effector DNA binding domain can be engineered to bind to a target DNA sequence. While not to be construed as limiting, TALENs useful for the methods provided herein include those described in WO10/079430 and U.S. Patent Application Publication No. 2011/0145940.

In some embodiments, the TAL effector DNA binding domain can comprise 10 or more DNA binding repeats, and preferably 15 or more DNA binding repeats. In some embodiments, each DNA binding repeat comprises a RVD that determines recognition of a base pair in the target DNA, and wherein each DNA binding repeat is responsible for recognizing one base pair in the target DNA. In some embodiments, the RVD comprises one or more of: HD for recognizing C; NG for recognizing T; NI for recognizing A; NN for recognizing G or A; NS for recognizing A or C or G or T; N* for recognizing C or T, where * represents a gap in the second position of the RVD; HG for recognizing T; H* for recognizing T, where * represents a gap in the second position of the RVD; IG for recognizing T; NK for recognizing G; HA for recognizing C; ND for recognizing C; HI for recognizing C; HN for recognizing G; NA for recognizing G; SN for recognizing G or A; and YG for recognizing T.

### Cas Endonuclease

In some embodiments, the targeted polypeptide used in methods and compositions of the disclosure is a clustered regularly interspaced short palindromic repeats (CRISPR)-associated (Cas) protein or a modified derivative. A Cas protein refers to an RNA-guided double-stranded DNA-binding nuclease protein or nickase protein. Wild-type Cas nuclease has two functional domains, e.g., RuvC and HNH, that cut different DNA strands. A Cas protein can induce double-strand breaks in genomic DNA (target nucleic acid) when both functional domains are active. The Cas protein can comprise one or more catalytic domains of a Cas protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the Cas protein can be a fusion protein, e.g., the two catalytic domains are derived from different bacteria species.

Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologs thereof, variants thereof, mutants thereof, and derivatives thereof. There are three main types of Cas proteins (type I, type II, and type III), and 10 subtypes including 5 type I, 3 type II, and 2 type III proteins (see, e.g., Hochstrasser and Doudna, Trends Biochem Sci, 2015:40(1):58-66). Type II Cas proteins include Cas1, Cas2, Csn2, Cas9, and Cfp1. These Cas proteins are known to those skilled in the art. For example, the amino acid sequence of the Streptococcus pyogenes wild-type Cas9 polypeptide is set forth, e.g., in NBCI Ref Seq. No. NP_269215, and the amino acid sequence of Streptococcus thermophilus wild-type Cas9 polypeptide is set forth, e.g., in NBCI Ref. Seq. No. WP_011681470.

Cas proteins, e.g., Cas9 nucleases, can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

In the embodiments described herein, the Cas protein is a nuclease-inactive variant. For example, useful variants of the Cas9 nuclease can include a single inactive catalytic domain, such as a RuvC- or HNH-enzyme or a nickase. A Cas9 nickase has only one active functional domain and can cut only one strand of the target nucleic acid, thereby creating a single strand break or nick. In some embodiments, the Cas9 nuclease can be a mutant Cas9 nuclease having one or more amino acid mutations. For example, the mutant Cas9 having at least a D10A mutation is a Cas9 nickase. In other embodiments, the mutant Cas9 nuclease having at least a H840A mutation is a Cas9 nickase. Other examples of mutations present in a Cas9 nickase include, without limitation, N854A and N863A. A double-strand break can be introduced using a Cas9 nickase if at least two DNA-targeting RNAs that target opposite DNA strands are used. A double-nicked induced double-strand break can be repaired by NHEJ or HDR (Ran et al., 2013, Cell, 154:1380-1389). Non-limiting examples of Cas9 nucleases or nickases are described in, for example, U.S. Pat. Nos. 8,895,308; 8,889,418; and 8,865,406 and U.S. Application Publication Nos. 2014/0356959, 2014/0273226 and 2014/0186919. The Cas9 nuclease or nickase can be codon-optimized for the target cell or target organism.

In some embodiments, a catalytically-inactive Cas protein variant can be a Cas9 polypeptide that contains two silencing mutations of the RuvC1 and HNH nuclease domains (D10A and H840A), which is referred to as dCas9 (Jinek et al., Science, 2012, 337:816-821; Qi et al., Cell, 152 (5): 1173-1183). In one embodiment, the dCas9 polypeptide from Streptococcus pyogenes comprises at least one mutation at position D10, G12, G17, E762, H840, N854, N863, H982, H983, A984, D986, A987 or any combination thereof. Descriptions of such dCas9 polypeptides and variants thereof are provided in, for example, International Patent Publication No. WO 2013/176772. The dCas9 enzyme can contain a mutation at D10, E762, H983, or D986, as well as a mutation at H840 or N863. In some instances, the dCas9 enzyme can contain a D10A or D10N mutation. Also, the dCas9 enzyme can contain a H840A, H840Y, or H840N. In some embodiments, the dCas9 enzyme can contain D10A and H840A; D10A and H840Y; D10A and H840N; D10N and H840A; D10N and H840Y; or DION and H840N substitutions. The substitutions can be conservative or non-conservative substitutions to render the Cas9 polypeptide catalytically inactive and able to bind to target nucleic acid. In methods and compositions described herein, a catalytically-inactive Cas protein (e.g., dCas9) linked to a transcriptional activator domain (e.g., VP64), can be used to target a promoter or enhancer sequence of the gene of interest in order to upregulate the expression of the gene.

As described herein, the targeted polypeptide can be a catalytically-inactive CRISPR nuclease. In some embodiments, the targeted polypeptide is dCas9. The CRISPR activation (CRISPRa) system utilizes a catalytically-inactive CRISPR nuclease (e.g. dCas9) linked to one or more transcriptional activators to upregulate expression of the genes of interest. CRISPRa further includes a gRNA targeting a portion of the promoter or enhancer sequence of the gene whose expression is to be upregulated. In some embodiments, transcriptional activators, fused to a catalytically-inactive nuclease or a gRNA, can recruit transcriptional factors or RNA polymerase close to the coding sequence of the gene of interest. For example, the VP64 transcriptional activator or any other transcriptional activator can be fused to the catalytically-inactive nuclease to increase transcription of the target gene.

In some embodiments, upregulating the expression of the target gene in a cell can include introducing into the cell a catalytically-inactive nuclease (e.g., a dCas9) linked to a transcriptional activator domain (e.g., VP64) and a gRNA that targets a portion of a promoter sequence operably linked to a coding sequence of the target gene. In certain embodiments, the gRNA targets the promoter sequence comprising the nucleic acid sequence selected from SEQ ID NOs: 78-114 or 116 which operably linked to a coding sequence of the protein comprising the amino acid sequence selected from SEQ ID NOs: 57-77 or 115, and can comprise a sequence having at least 90%, 95%, 98%, 99% or 100% identity to a sequence selected from SEQ ID NOs: 1-55 or as shown in Table S2. For example, a promoter sequence operably linked to a coding sequence of the UCP1 protein (SEQ ID NO: 57) can comprise the sequence of SEQ ID NO: 78. In specific embodiments, the gRNA targeting the promoter sequence operably linked to a coding sequence of the UCP1 protein (SEQ ID NO: 57) can comprise a sequence having at least 90%, 95%, 98%, 99% or 100% identity to a sequence of any one of SEQ ID NOS: 1-6.

Further, upregulating the expression of the target gene in a cell can include introducing into the cell a catalytically-inactive nuclease (e.g., a dCas9) linked to a transcriptional activator domain (e.g., VP64) and a gRNA that targets a portion of an enhancer sequence operably linked to a coding sequence of the target gene.

A Cas protein can be guided to its target nucleic acid by a guide RNA (gRNA). A gRNA is a version of the naturally occurring two-piece guide RNA (crRNA and tracrRNA) engineered into a two-piece gRNA or a single, continuous sequence. A gRNA can contain a guide sequence (e.g., the crRNA equivalent portion of the gRNA) that targets the Cas protein to the target nucleic acid and a scaffold sequence that interacts with the Cas protein (e.g., the tracrRNAs equivalent portion of the gRNA). A gRNA can be selected using a software. As a non-limiting example, considerations for selecting a gRNA can include, e.g., the PAM sequence for the Cas protein to be used, and strategies for minimizing off-target modifications. Tools, such as NUPACK® and the CRISPR Design Tool, can provide sequences for preparing the gRNA, for assessing target modification efficiency, and/or assessing cleavage at off-target sites.

The guide sequence in the gRNA may be complementary to a specific sequence within a target nucleic acid (e.g., the UCP1 promoter sequence or the UPP1 promoter). The 3′ end of the target nucleic acid sequence can be followed by a PAM sequence. Approximately 20 nucleotides upstream of the PAM sequence is the target nucleic acid. In general, a Cas9 protein or a variant thereof cleaves about three nucleotides upstream of the PAM sequence. The guide sequence in the gRNA can be complementary to either strand of the target nucleic acid.

In some embodiments, the guide sequence of a gRNA comprises about 100 nucleic acids at the 5′ end of the gRNA that can direct the Cas protein to the target nucleic acid site using RNA-DNA complementarity base pairing. In some embodiments, the guide sequence comprises about 20 nucleic acids at the 5′ end of the gRNA that can direct the Cas protein to the target nucleic acid site using RNA-DNA complementarity base pairing. In other embodiments, the guide sequence comprises less than 20, e.g., 19, 18, 17, 16, 15 or less, nucleic acids that are complementary to the target nucleic acid site. In some instances, the guide sequence in the gRNA contains at least one nucleic acid mismatch in the complementarity region of the target nucleic acid site. In some instances, the guide sequence contains about 1 to about 10 nucleic acid mismatches in the complementarity region of the target nucleic acid site.

The scaffold sequence in the gRNA can serve as a protein-binding sequence that interacts with the Cas protein or a variant thereof. In some embodiments, the scaffold sequence in the gRNA can comprise two complementary stretches of nucleotides that hybridize to one another to form a double-stranded RNA duplex (dsRNA duplex). The scaffold sequence may have structures such as lower stem, bulge, upper stem, nexus, and/or hairpin. In some embodiments, the scaffold sequence in the gRNA can be between about 90 nucleic acids to about 120 nucleic acids, e.g., about 90 nucleic acids to about 115 nucleic acids, about 90 nucleic acids to about 110 nucleic acids, about 90 nucleic acids to about 105 nucleic acids, about 90 nucleic acids to about 100 nucleic acids, about 90 nucleic acids to about 95 nucleic acids, about 95 nucleic acids to about 120 nucleic acids, about 100 nucleic acids to about 120 nucleic acids, about 105 nucleic acids to about 120 nucleic acids, about 110 nucleic acids to about 120 nucleic acids, or about 115 nucleic acids to about 120 nucleic acids.

In some embodiments, the gRNAs used in methods and compositions of the disclosure target a portion of a promoter or enhancer sequence operably linked to a coding sequence of the gene of interest. The catalytically-inactive nuclease (e.g., a dCas9) can be linked to a transcriptional activator domain (e.g. VP64). In certain embodiments, the gRNA targets the promoter sequence comprising the sequence of SEQ ID NO: 78-114 or 116 and can comprise a sequence having at least 90%, 95%, 98%, 99% or 100% identity to a sequence of any one of SEQ ID NOS: 1-55 or as shown in Table S2.

In some embodiments, the catalytically-inactive nuclease (e.g., a dCas9) linked to a transcriptional activator domain (e.g. VP64), can be guided by one gRNA and upregulate the expression of the gene of interest. In some embodiments, the catalytically-inactive nuclease (e.g., a dCas9) linked to a transcriptional activator domain (e.g. VP64), can be guided by multiple gRNAs in the same cell and in some embodiments upregulate the expression of multiple genes of interest.

### Transcriptional Activators

Transcriptional activators are protein domains or whole proteins linked to a targeted polypeptide to assist in the recruitment of important co-factors as well as RNA Polymerase for transcription of the gene(s) targeted by the system. In order for a protein to be made from the gene that encodes it, RNA polymerase must make RNA from the DNA template of the gene during a process called transcription. Transcriptional activators have a DNA binding domain and a domain for activation of transcription. The activation domain can recruit general transcription factors or RNA polymerase to the gene sequence. Activation domains can also function by facilitating transcription by stalled RNA polymerases, and in eukaryotes can act to move nucleosomes on the DNA or modify histones to increase gene expression. These activators can be introduced into the system through attachment to a targeted polypeptide (e.g., dCas9). As used herein, the term “transcriptional activator,” “transcriptional activation domain,” and “transcriptional activator domain” are used interchangeably.

The transcriptional activator used in methods and compositions of the disclosure is linked to a targeted polypeptide which targets a portion of a promoter or enhancer sequence operably linked to a gene of interest. Examples of transcriptional activators include, but are not limited to, HSF1, VP16, VP48, VP64, VP160, p53, p65, RTA, MyoD1, SET7, LSD1, CIB1, AD2, CR3, GATA4, SP1, MEF2C, TAX, SET9, VP64-p65-RTA (VPR), Histone Acetyltransferase p300, TET1 Hydroxylase Catalytic Domain, Synergistic activation mediator (SAM), SunTag, and PPAR-gamma. In some embodiments, the transcriptional activator is VP64. In one embodiment, the transcriptional activator is VP64, and the targeted polypeptide is catalytically-inactive nuclease dCas9.

The targeted polypeptide can be linked to one or more transcriptional activators. In some embodiments, the targeted polypeptide (e.g., dCas9) linked to one transcriptional activator (e.g., VP64). In some embodiments, the targeted polypeptide (e.g., dCas9) linked to two or more transcriptional activators (e.g., VP64-p65-RTA). The VP64-p65-RTA, or VPR, dCas9 activator was created by modifying an existing dCas9 activator, in which a Vp64 transcriptional activator is joined to the C terminus of dCas9. In the dCas9-VPR protein, the transcription factors p65 and RTA are added to the C terminus of dCas9-Vp64. Therefore, all three transcription factors are targeted to the same gene.

The targeted polypeptide can be directly, or indirectly, linked to a transcriptional activator. In some embodiments, the targeted polypeptide directly linked to a transcriptional activator as a fusion protein (e.g., dCas9-VP64). In some embodiments, the targeted polypeptide indirectly linked to a transcriptional activator through a gRNA (e.g., dCas9-SAM). In the Synergistic Activation Mediator (SAM) system, dCas9-VP64 recruits more transcriptional factors (MS2, p65, and HSF1) working synergistically to activate the gene of interest. In order to assemble different transcriptional activators, the dCas9-SAM system uses a modified single guide RNA (sgRNA) that has binding sites for the MS2 protein. Hairpin aptamers are attached to the tetra loop and the stem loop 2 of the sgRNA to become binding sites for dimerized MS2 bacteriophage coat proteins. As the hairpins are exposed outside of the dCas9-sgRNA complex, other transcriptional factors can bind to the MS2 protein without disrupting the dCas9-sgRNA complex. Thus, the MS2 protein is engineered to include p65 and HSF1 proteins. The MS2-p65-HSF1 fusion protein interacts with the dCas9-VP64 to recruit more transcriptional factors onto the promoter of the target genes.

In some embodiments, transcriptional activator domains (such as VP16, VP48, VP64, VP160 and the like) can be fused to the zinc-finger binding domain. For example, engineered zinc finger transcriptional activator that interact with a promoter region of the gamma-globulin gene was shown to enhance fetal hemoglobin production in primer adult erythroblasts (Wilber et al., Blood, 115(15):3033-3041). Other polydactyl zinc-finger transcription factors are also known in the art, including those disclosed in Beerli and Barbas (see, Nature Technology, (2002) 20:135-141).

In some embodiments, the TALEN is engineered such that the TAL effector comprises one or more transcriptional activator domains (e.g., VP16, VP48, VP64 or VP160). For example, engineered TAL effectors having a transcriptional activator domain at the c-terminus of the TAL effector were shown to modulate transcription of Sox2 and Klf4 genes in human 293 FT cells (Zhang et al., Nature Biotechnology, 29(2):149-153 (2011). Other TAL effector transcription factors (TALE-TFs) are also known in the art, including those disclosed in Perez-Pinera et al., (Nature Methods, (2013) 10(3):239-242) that demonstrated modulation of IL1RN, KLK3, CEACAM5 and ERBB2 genes in human 293T cells using TALE-TFs. In some embodiments, the one or more transcriptional activator domains are located adjacent to the nuclear localization signal (NLS) present in the C-terminus of the TAL effector. In another embodiment, the TALE-TFs can bind nearby sites upstream or downstream of the transcriptional start site (TSS) for a target gene.

### Heterologous Expression Cassette

In other embodiments, one or more gene products as described herein can be overexpressed in a cell as described herein. Overexpression of one or more gene products in a cell means the cell expresses more than normal level of the gene products a native cell expresses under the same condition. For example, a gene product can be overexpressed by introducing a heterologous expression cassette comprising a promoter operably linked to the gene of interest into the cell.

An expression cassette is a polynucleotide comprising at least a promoter operable linked to a coding sequence for a gene product as described herein. The expression cassette, when introduced into a cell, directs the cell's machinery to make RNA and protein(s). Some expression cassettes are designed for modular cloning of protein-encoding sequences so that the same cassette can easily be altered to make different proteins. An expression cassette is composed of one or more genes and the sequences controlling their expression. An expression cassette comprises three components: a promoter sequence, an open reading frame, and a 3′ untranslated region that, in eukaryotes, usually contains a polyadenylation site. Different expression cassettes can be transfected into different organisms including bacteria, yeast, plants, and mammalian cells as long as the correct regulatory sequences are used.

Exemplary heterologous expression cassettes used in methods and compositions of the disclosure include, for example, a promoter operably linked to a gene encoding a protein selected from the group consisting of Uncoupling protein 1 (UCP1/SLC25A7) (SEQ ID NO: 57 or a sequence having at least 90%, 95%, 98%, 99% thereto), PPARG coactivator 1 alpha (PPARGC1A/PCG1A) (SEQ ID NO: 58 or a sequence having at least 90%, 95%, 98%, 99% thereto), PR/SET domain 16 (PRDM16) (SEQ ID NO: 59 or a sequence having at least 90%, 95%, 98%, 99% thereto), FFAR1/GPR40 (SEQ ID NO: 60 or a sequence having at least 90%, 95%, 98%, 99% thereto), FFAR4/GPR120 (SEQ ID NO: 61 or a sequence having at least 90%, 95%, 98%, 99% thereto), FOXO1 (SEQ ID NO: 62 or a sequence having at least 90%, 95%, 98%, 99% thereto), GLUT1 (SEQ ID NO: 63 or a sequence having at least 90%, 95%, 98%, 99% thereto), GLUT2 (SEQ ID NO: 64 or a sequence having at least 90%, 95%, 98%, 99% thereto), GLUT3 (SEQ ID NO: 65 or a sequence having at least 90%, 95%, 98%, 99% thereto), GLUT4 (SEQ ID NO: 66 or a sequence having at least 90%, 95%, 98%, 99% thereto), CHREBP (SEQ ID NO: 67 or a sequence having at least 90%, 95%, 98%, 99% thereto), CD36 (SEQ ID NO: 68 or a sequence having at least 90%, 95%, 98%, 99% thereto), CTP1B (SEQ ID NO: 69 or a sequence having at least 90%, 95%, 98%, 99% thereto), AIFM2/FSP1 (SEQ ID NO: 70 or a sequence having at least 90%, 95%, 98%, 99% thereto), GSK (SEQ ID NO: 71 or a sequence having at least 90%, 95%, 98%, 99% thereto), IRS1 (SEQ ID NO: 72), DGAT1 (SEQ ID NO: 73 or a sequence having at least 90%, 95%, 98%, 99% thereto), DGAT2 (SEQ ID NO: 74 or a sequence having at least 90%, 95%, 98%, 99% thereto), ACACA (SEQ ID NO: 75 or a sequence having at least 90%, 95%, 98%, 99% thereto), UPP1 (SEQ ID NO:115 or a sequence having at least 90%, 95%, 98%, 99% thereto), CKB (SEQ ID NO: 117 or a sequence having at least 90%, 95%, 98%, 99% thereto) or a polypeptide of Table 2 or a sequence having at least 90%, 95%, 98%, 99% thereto. Promoters can be selected as desired. In some embodiments, the promoters are inducible or constitutively expressed.

### Downregulating Gene Expression

The disclosure further provides compositions and methods for preventing and treating metabolic diseases, diabetes, and cancers through downregulating expression of the genes associated with glucose and lipid metabolism, i.e., ACACB or NRIP1, in one or more cells (e.g., in mesenchymal stem cells, pre-adipocytes, fat cells, adipocytes, adipose organoids or fat progenitor cells) in the subject. Techniques to downregulate the expression of the gene can include, but are not limited to, e.g., CRISPR interference (CRISPRi), RNA interference (RNAi), and antisense therapy.

### CRISPR Interference (CRISPRi)

CRISPRi utilizes a catalytically-inactive nuclease containing one or more amino acid mutations relative to a wild-type CRISPR nuclease and sterically represses transcription by blocking either transcriptional initiation or elongation. CRISPRi further includes a gRNA targeting a promoter or enhancer sequence of the gene whose expression is to be reduced. In eukaryotes, CRISPRi can also repress transcription via an effector domain. The catalytically-inactive nuclease can be linked to a repressor domain to allow transcription to be further repressed. For example, the well-studied Krüppel associated box (KRAB) domain can be fused to the catalytically-inactive nuclease to repress transcription of the ACACB or NRIP1 gene. In certain embodiments, the catalytically-inactive nuclease is a dead Cas9 (dCas9). Other examples of catalytically-inactive nucleases are described in detail further herein.

In some embodiments, methods for downregulating expression of certain genes associated with glucose and lipid metabolism can include introducing into the one or more cells of the subject a catalytically-inactive nuclease (e.g., a dCas9) linked to a transcriptional repressor domain (e.g., KRAB) and a gRNA that targets a portion of a promoter sequence operably linked to a coding sequence of the endogenous ACACB or NRIP1 gene.

Further, methods for downregulating expression of certain genes can include introducing into the one or more cells of the subject a catalytically-inactive nuclease (e.g., a dCas9) linked to a transcriptional repressor domain (e.g., KRAB) and a gRNA that targets a portion of an enhancer sequence operably linked to a coding sequence of the ACACB or NRIP1 gene.

### RNA Interference (RNAi)

RNA interference (RNAi) is a biological process in which RNA molecules (i.e., inhibitory RNA polynucleotides) are involved in sequence-specific suppression of gene expression. An inhibitory RNA polynucleotide can be synthesized to target the ACACB or NRIP1 gene to lower its expression level. The inhibitory RNA polynucleotide can be of various lengths, e.g., between 15 and 30 nucleotides (e.g., 15, 16, 17, 18, 19, 20, 21, 22, 23, 24, 25, 26, 27, 28, 29, or 30 nucleotides). In further embodiments, the inhibitory RNA polynucleotide can be single-stranded or double-stranded. The inhibitory RNA polynucleotide can specifically hybridize to or is complementary (e.g., partially complementary) to a portion of the ACACB or NRIP1 gene, such that stable and specific binding occurs between the inhibitory RNA polynucleotide and the gene. There is a sufficient degree of complementarity between the inhibitory RNA polynucleotide and the ACACB or NRIP1 gene to avoid non-specific binding of the inhibitory RNA polynucleotide to non-target sequences.

The inhibitory RNA polynucleotides described herein can be a microRNA, which is a single-stranded RNA molecule of about 21-23 nucleotides (e.g., 21, 22, or 23 nucleotides) in length. miRNAs are encoded by genes from whose DNA they are transcribed, but miRNAs are not translated into protein (non-coding RNA); instead, each primary transcript (a pri-miRNA) is processed into a short stem-loop structure called a pre-miRNA and finally into a functional mature miRNA. Mature miRNA molecules are either partially or completely complementary to one or more messenger RNA (mRNA) molecules.

miRNAs are first transcribed as primary transcripts or pri-miRNA with a cap and poly-A tail and processed to short, nucleotide stem-loop structures known as pre-miRNA in the cell nucleus by a protein complex known as the Microprocessor complex, consisting of the nuclease Drosha and the double-stranded RNA binding protein Pasha (Denli et al., Nature, 432:231-235, 2004). These pre-miRNAs are then processed to mature miRNAs in the cytoplasm by interaction with the endonuclease Dicer, which also initiates the formation of the RNA-induced silencing complex (RISC) (Bernstein et al., Nature, 409:363-366, 2001). Either the sense strand or antisense strand of DNA can function as templates to give rise to miRNA. When Dicer cleaves the pre-miRNA stem-loop, two complementary short RNA molecules are formed, but only one is integrated into the RISC complex. This strand is known as the guide strand and is selected by the argonaute protein, which is the catalytically active RNase in the RISC complex, on the basis of the stability of the 5′ end (Preall et al., Curr. Biol., 16:530-535, 2006). The remaining strand, known as the anti-guide or passenger strand, is degraded as a RISC complex substrate). After integration into the active RISC complex, miRNAs base pair with their complementary mRNA molecules and induce target mRNA degradation and/or translational silencing.

Mammalian miRNA molecules are usually complementary to a site in the 3′ UTR of the target mRNA sequence (e.g., a portion of the ACACB or NRIP1 mRNA). In some embodiments, the annealing of the miRNA to the target mRNA inhibits protein translation by blocking the protein translation machinery. In some embodiments, the annealing of the miRNA to the target mRNA facilitates the cleavage and degradation of the target mRNA.

The inhibitory RNA polynucleotides described herein can also be a small interfering RNA (siRNA), which refers to a double stranded RNA with the two complementary strands each having between 15 and 20 nucleotides (e.g., 15, 16, 17, 18, 19, or 20 nucleotides). In some embodiments, the two strands of an siRNA molecule can each have a 3′-end overhang of two or three nucleotides. In an siRNA molecule, one strand (e.g., the antisense strand) is guiding and complementary (e.g., partially complementary) to the target gene (e.g., the ACACB or NRIP1 gene).

Suitable siRNA sequences can be identified using methods known in the art. For example, prediction algorithms that predict potential siRNA-targets based upon complementary DNA sequences in the target genes are available in the art. TargetScanHuman, for example, is a comprehensive web resource for inhibitory RNA-target predictions, and uses an algorithm that incorporates current biological knowledge of inhibitory RNA-target rules including seed-match model, evolutionary conservation, and free binding energy (Li and Zhang, Wiley Interdiscip Rev RNA 6:435-452, 2015 and Agarwal et al., Elife 4, 2015). In some embodiments, to further enhance silencing efficiency of the siRNA sequences, potential siRNA sequences may be analyzed to identify sites that do not contain regions of homology to other coding sequences, e.g., in the target cell or organism.

Once a potential siRNA sequence has been identified, a complementary sequence (i.e., an antisense strand sequence) can be designed. A potential siRNA sequence can also be analyzed using a variety of criteria known in the art. For example. to enhance their silencing efficiency, the siRNA sequences may be analyzed by a rational design algorithm to identify sequences that have one or more of the following features: (1) G/C content of about 25% to about 60% G/C; (2) at least 3 A/Us at positions 15-19 of the sense strand; (3) no internal repeats; (4) an A at position 19 of the sense strand; (5) an A at position 3 of the sense strand; (6) a U at position 10 of the sense strand; (7) no G/C at position 19 of the sense strand; and (8) no G at position 13 of the sense strand. The siRNA design tools that incorporate algorithms that assign suitable values of each of these features and are useful for selection of the siRNA are available in the art. One of skill in the art will appreciate that sequences with one or more of the foregoing characteristics may be selected for farther analysis and testing as potential siRNA sequences.

The inhibitory RNA polynucleotides described herein can also be a small hairpin RNA or short hairpin RNA (shRNA), which is a short RNA sequence that makes a tight hairpin turn that can be used to silence gene expression via RNA interference. The shRNA hairpin structure is cleaved by the cellular machinery into siRNA, which is then bound to the RNA-induced silencing complex (RISC). In some embodiments, shRNAs can be between 15 to 60 nucleotides (e.g., 15, 20, 25, 30, 35, 40, 45, 50, 55, or 60 nucleotides) in length. Non-limiting examples of shRNA include a double-stranded polynucleotide molecule assembled from a single-stranded molecule, in which the sense and antisense regions are linked by a nucleic acid-based or non-nucleic acid-based linker; and a double-stranded polynucleotide molecule with a hairpin secondary structure having self-complementary sense and antisense regions.

The complementarity between an siRNA or shRNA and its corresponding target sequence may be 100%. In some embodiments, the complementarity between the siRNA or shRNA and its corresponding target sequence is less than 100%, although 100% complementarity is desired to avoid off-target effects. In some embodiments, the complementarity between the siRNA or shRNA and its corresponding target sequence is at least 95%, at least 90%, at least 85%, or at least 80%.

In some embodiments, the siRNA or shRNA comprises one or more modified nucleotides. In some embodiments, the modified nucleotide of the siRNA or shRNA comprises a sugar modification, a nucleic acid base modification, and/or a phosphate backbone modification. Exemplary modifications are described further below. In one particular embodiment, the siRNA or shRNA includes one or more locked nucleic acids (LNA). A locked nucleic acid is a nucleotide having a modified ribose moiety in which the ribose moiety comprises an extra bridge connecting the 2′ and 4′ carbons. See e.g., Elmen et al. (2005), Nucleic Acids Research 33 (1): 439-447; Mook et al. (2007), Mol Canc Ther 6 (3): 833-843; Grunweller et al. (2003), Nucleic Acids Research 31 (12): 3185-3193).

In some embodiments, the siRNA or shRNA comprises an overhang on either the sense strand or the antisense strand or both (e.g., on each 3′ end of both strands). In some embodiments, siRNA or shRNA includes an overhang on both the sense and the antisense strand. The overhang may be at either the 5′ end or the 3′ end of the strand. In some embodiments, both the 5′ end and the 3′ end comprise an overhang. The overhang can have any nucleotide sequence and may be 1-10 nucleotides in length. In some embodiments, the overhang is 2-6 nucleotides in length. In some embodiments, the overhang is 2-4 nucleotides in length. In some cases, the overhang comprises modified nucleotides. For example, the overhang may include locked nucleic acids (LNAs).

### Antisense Therapy

Antisense therapy is a gene expression suppression technique that uses antisense oligonucleotides (ASOs) to target mRNAs (e.g., ACACB or NRIP1 mRNA). Antisense therapy uses a naturally occurring enzyme, RNase H, that has the activity of destroying the RNA strand of an RNA:DNA duplex. In antisense therapy, a DNA oligonucleotide of approximately 15-30 bases in length that was complimentary to the target gene is introduced into the cells. Once inside the cell, the oligonucleotide base pairs with its targeted region in the target gene and when RNase H binds to the resulting duplex, it cleaves the RNA in multiple places, leaving the DNA oligonucleotide intact to help catalyze destruction of more target mRNAs. In this way, the production of the protein encoded by the targeted mRNA is greatly reduced.

In some embodiments, the antisense polynucleotide is 8-100, e.g., 12-50, e.g., 16-30 nucleotides in length. The complementarity between an antisense polynucleotide and its corresponding target sequence may be 100%. In some embodiments, the complementarity between the antisense polynucleotide and its corresponding target sequence is less than 100%, although 100% complementarity is desired to avoid off-target effects. In some embodiments, the complementarity between the antisense polynucleotide and its corresponding target sequence is at least 95%, at least 90%, at least 85%, or at least 80%.

In some embodiments, the antisense oligonucleotide comprises one or more modified nucleotides. In some embodiments, the modified nucleotide comprises a sugar modification, a nucleic acid base modification, and/or a phosphate backbone modification. Exemplary modifications are described further below. In one particular embodiment, the antisense polynucleotide is designed as a gapmer comprising a central stretch (gap) of nucleotides capable of inducing RNase H cleavage, and two flanking regions containing one or more modified nucleosides. Gapmer structures are well characterized and may be designed using known methods in the art, see, e.g., Monia et al. (1993), “Evaluation of 2 “-modified oligonucleotides containing 2-” deoxy gaps as antisense inhibitors of gene expression”, J. Biol. Chem.; 268:14514-14522; Deleavey et al. (2012), “Designing chemically modified oligonucleotides for targeted gene silencing”, Chem. Biol.; 19:937-954; and Stanley T. Crooke (2008), “Antisense Drug Technology-Principles, Strategies, and Applications”, 2nd Edition, CRC press. Accordingly, in some aspects, the antisense polynucleotide is a gapmer. In some embodiments, the antisense polynucleotide is a locked nucleic acid (LNA) gapmer, where the modified nucleotides in the flanking regions are LNA nucleotides. In some embodiments, the antisense polynucleotide is a mixmer comprising alternating stretches of LNA and unmodified nucleotides, see e.g. U.S. Pat. Nos. 5,013,830; 5,149,797; 5,220,007; 5,256,775, each of which is herein incorporated by reference. In one embodiment, the antisense polynucleotide is a headmer comprising only a flanking region at the 5′ terminus. In another embodiment, the antisense polynucleotide is a tailmer comprising only a flanking region at the 3′ terminus.

### Polynucleotides

The present disclosure further provides a polynucleotide comprising one or more expression cassettes encoding a targeted polypeptide, wherein the targeted polypeptide linked to a transcriptional activator targets a portion of a promoter or enhancer sequence operably linked to a polynucleotide encoding a polypeptide selected from the group consisting of Uncoupling protein 1 (UCP1/SLC25A7) (SEQ ID NO: 57 or a sequence having at least 90%, 95%, 98%, 99% thereto), PPARG coactivator 1 alpha (PPARGC1A/PCG1A) (SEQ ID NO: 58 or a sequence having at least 90%, 95%, 98%, 99% thereto), PR/SET domain 16 (PRDM16) (SEQ ID NO: 59 or a sequence having at least 90%, 95%, 98%, 99% thereto), FFAR1/GPR40 (SEQ ID NO: 60 or a sequence having at least 90%, 95%, 98%, 99% thereto), FFAR4/GPR120 (SEQ ID NO: 61 or a sequence having at least 90%, 95%, 98%, 99% thereto), FOXO1 (SEQ ID NO: 62 or a sequence having at least 90%, 95%, 98%, 99% thereto), GLUT1 (SEQ ID NO: 63 or a sequence having at least 90%, 95%, 98%, 99% thereto), GLUT2 (SEQ ID NO: 64 or a sequence having at least 90%, 95%, 98%, 99% thereto), GLUT3 (SEQ ID NO: 65 or a sequence having at least 90%, 95%, 98%, 99% thereto), GLUT4 (SEQ ID NO: 66 or a sequence having at least 90%, 95%, 98%, 99% thereto), CHREBP (SEQ ID NO: 67 or a sequence having at least 90%, 95%, 98%, 99% thereto), CD36 (SEQ ID NO: 68 or a sequence having at least 90%, 95%, 98%, 99% thereto), CTP1B (SEQ ID NO: 69 or a sequence having at least 90%, 95%, 98%, 99% thereto), AIFM2/FSP1 (SEQ ID NO: 70 or a sequence having at least 90%, 95%, 98%, 99% thereto), GSK (SEQ ID NO: 71 or a sequence having at least 90%, 95%, 98%, 99% thereto), IRS1 (SEQ ID NO: 72 or a sequence having at least 90%, 95%, 98%, 99% thereto), DGAT1 (SEQ ID NO: 73 or a sequence having at least 90%, 95%, 98%, 99% thereto), DGAT2 (SEQ ID NO: 74 or a sequence having at least 90%, 95%, 98%, 99% thereto), ACACA (SEQ ID NO: 75 or a sequence having at least 90%, 95%, 98%, 99% thereto), UPP1 (SEQ ID NO: 115 or a sequence having at least 90%, 95%, 98%, 99% thereto), CKB (SEQ ID NO: 117 or a sequence having at least 90%, 95%, 98%, 99% thereto) or any polypeptide from Table 2 or at least 90%, 95%, 98%, 99% thereto, wherein the composition upregulates expression of the at least one polypeptide when introduced to a cell. In some embodiments, the polynucleotide comprises an expression cassette comprising a promoter operably linked to a nucleic acid encoding the catalytically-inactive nuclease.

In some embodiments, the polynucleotide comprises an expression cassette comprising a promoter operably linked to a nucleic acid encoding the catalytically-inactive nuclease fused with a transcriptional activator. In certain embodiments, the polynucleotide comprises an expression cassette comprising a promoter operably linked to a nucleic acid encoding the catalytically-inactive nuclease fused with a transcriptional activator through a linker (SEQ ID NO: 56).

In some embodiments, a polynucleotide comprising one or more expression cassettes encoding a targeted polypeptide, wherein the targeted polypeptide linked to a transcriptional repressor targets a portion of a promoter or enhancer sequence operably linked to a polynucleotide encoding a polypeptide of ACACB (e.g., SEQ ID NO: 76 or a sequence having at least 90%, 95%, 98%, 99% thereto) or NRIP1 (e.g., SEQ ID NO: 77 or a sequence having at least 90%, 95%, 98%, 99% thereto), wherein the composition downregulates expression of the at least one polypeptide when introduced to a cell.

In some embodiments, the polynucleotide comprises (i) a first expression cassette comprising a first promoter operably linked to a first nucleic acid encoding the gRNA and (ii) a second expression cassette comprising a second promoter operably linked to a second nucleic acid encoding the catalytically-inactive nuclease. In some embodiments, the polynucleotide comprises (i) a first expression cassette comprising a first promoter operably linked to a first nucleic acid encoding the gRNA and (ii) a second expression cassette comprising a second promoter operably linked to a second nucleic acid encoding the catalytically-inactive nuclease the transcriptional activator.

### Delivery

In compositions and methods described herein, the targeted polypeptide and its associated components (e.g., gRNA, transcriptional activator), or the polynucleotides comprising expression cassettes encoding the targeted polypeptide and its associated components, or the polynucleotide comprising the heterologous expression cassette encoding the gene of interest, can be delivered into one or more cells (e.g., adipocytes, adipose stem cells or adipose progenitor cells) of the subject using a number of techniques in the art.

### Viral Vector

In some embodiments, the targeted polypeptide (e.g., dCas9) linked to the transcriptional activator, and optionally a gRNA, can be introduced into the cell via viral delivery using a viral vector. For example, viral vectors can be based on vaccinia virus, poliovirus, retrovirus, lentivirus, adenovirus, adeno-associated virus (AAV) (e.g., recombinant AAV (rAAV)), SV40, herpes simplex virus, human immunodeficiency virus, and the like. A retroviral vector can be based on Murine Leukemia Virus, spleen necrosis virus, and vectors derived from retroviruses such as Rous Sarcoma Virus, Harvey Sarcoma Virus, avian leukosis virus, a lentivirus (e.g., integration deficient lentivirus), human immunodeficiency virus, myeloproliferative sarcoma virus, mammary tumor virus, and the like. In some embodiments, a retroviral vector can be an integration deficient gamma retroviral vector. In some embodiments, viral vectors can be virus-like particles. Other useful expression vectors are known to those of skill in the art, and many are commercially available. The following exemplary vectors are provided by way of example for eukaryotic host cells: pXT1, pSG5, pSVK3, pBPV, pMSG, and pSVLSV40. Examples of techniques that may be used to introduce a viral vector into a cell include, but not limited to, viral or bacteriophage infection, transfection, protoplast fusion, lipofection, calcium phosphate precipitation, polyethyleneimine (PEI)-mediated transfection, DEAE-dextran mediated transfection, liposome-mediated transfection, calcium phosphate precipitation, nanoparticle-mediated nucleic acid delivery, and the like.

### Electroporation

In some embodiments, the composition can be introduced into the cell via physical method transfection, such as electroporation, microinjection, gene gun, impalefection, hydrostatic pressure, continuous infusion, and sonication, etc. In some embodiments, a ribonucleoprotein (RNP) complex containing a Cas protein (e.g., Cas9 nuclease) and a gRNA can be formed first, then electroporated into the cell. Methods, compositions, and devices for electroporation are available in the art, e.g., those described in WO2006/001614 or Kim, J. A. et al. Biosens. Bioelectron. 23, 1353-1360 (2008). Additional or alternative methods, compositions, and devices for electroporation can include those described in U.S. Patent Appl. Pub. Nos. 2006/0094095; 2005/0064596; or 2006/0087522. Additional or alternative methods, compositions, and devices for electroporation can include those described in Li, L. H. et al. Cancer Res. Treat. 1, 341-350 (2002); U.S. Pat. Nos. 6,773,669; 7,186,559; 7,771,984; 7,991,559; 6,485,961; and 7,029,916; and U.S. Patent Appl. Pub. Nos: 2014/0017213; and 2012/0088842. Additional or alternative methods, compositions, and devices for electroporation can include those described in Geng, T. et al. J. Control Release 144, 91-100 (2010); and Wang, J., et al. Lab Chip 10, 2057-2061 (2010).

### DNA Transfection

In some embodiments, the composition can be introduced into the cell via chemical method transfection, such as calcium phosphate, cationic polymers (DEAE-dextran or polyethylenimine (PEI)), Lipofection (or liposome transfection), Fugene, and Dendrimer, etc. Lipofection is a lipid-mediated DNA-transfection process utilizing liposome vectors.

### Cells

The present disclosure provides a cell comprising a targeted polypeptide linked to a transcriptional activator, wherein the targeted polypeptide targets a portion of a promoter or enhancer sequence operably linked to a gene of interest, wherein the targeted polypeptide upregulates expression of the gene of interest in the cell.

The present disclosure also provides a cell comprising a polynucleotide of one or more expression cassettes encoding a targeted polypeptide, wherein the targeted polypeptide linked to a transcriptional activator, wherein the targeted polypeptide targets a portion of a promoter or enhancer sequence operably linked to a gene of interest, wherein the targeted polypeptide upregulates expression of the gene of interest in the cell.

The present disclosure also provides a cell comprising a heterologous expression cassette, wherein the heterologous expression cassette comprises a promoter operably linked to a gene of interest, wherein the heterologous expression cassette overexpresses the gene of interest in the cell.

As disclosed herein, the genes of interest in present disclosure include, but not limited to, Uncoupling protein 1 (UCP1/SLC25A7), PPARG coactivator 1 alpha (PPARGC1A/PCG1A), PR/SET domain 16 (PRDM16), FFAR1/GPR40, FFAR4/GPR120, FOXO1, GLUT1, GLUT2, GLUT3, GLUT4, CHREBP, CD36, CTP1B, AIFM2/FSP1, GSK, IRS1, DGAT1, DGAT2, ACACA, ACACB, UPP1, CKB and NRIP1 and as shown in Table 2.

In compositions and methods described herein, the targeted polypeptide and its associated components (e.g., gRNA, transcriptional activator), or the polynucleotides comprising expression cassettes encoding the targeted polypeptide and its associated components, or the polynucleotide comprising the heterologous expression cassette encoding the gene of interest, can be delivered into different organisms including bacteria, yeast, plants, and mammalian cells. In some embodiments, the cell is an adipocyte. In some embodiments, the cell is an adipose stem cell. In some embodiments, the cell is an adipose progenitor cell. In some embodiments, the cell is a white adipose cell.

Also provided is an engineered adipocyte or adipose stem cell that comprises a targeted polypeptide linked to a transcriptional activator, wherein the targeted polypeptide targets a portion of a promoter or enhancer sequence operably linked to a gene of interest, wherein the targeted polypeptide upregulates expression of the gene of interest compared to a wild-type adipocyte or adipose stem cell.

In some embodiments, the engineered adipocyte or adipose stem cell comprises a catalytically inactive nuclease linked to a transcriptional activator, wherein a catalytically inactive nuclease targets a portion of a promoter or enhancer sequence operably linked to at least one gene of interest. In some embodiments, the catalytically inactive nuclease is (i) a clustered regularly interspaced short palindromic repeats (CRISPR) nuclease, and the engineered adipocyte or adipose stem cell further comprises a gRNA that targets the portion of the promoter or enhancer sequence; (ii) a zinc finger nuclease (ZFN); or (iii) a transcription activator-like effector nuclease (TALEN). In some embodiments, the engineered adipocyte or adipose stem cell comprising a catalytically inactive clustered regularly interspaced short palindromic repeats (CRISPR) nuclease linked to a transcriptional activator. In some embodiments, the engineered adipocyte or adipose stem cell comprising a catalytically inactive dCas9 linked to a transcriptional activator. In some embodiments, the engineered adipocyte or adipose stem cell comprising a catalytically inactive dCas9 linked to a transcriptional activator VP64.

Also provided is an engineered adipocyte or adipose stem cell comprises a heterologous expression cassette, wherein the heterologous expression cassette comprises a promoter operably linked to a gene of interest, wherein the heterologous expression cassette upregulates expression of the gene of interest compared to a wild-type adipocyte or adipose stem cell. In some embodiments, the heterologous expression cassette is inserted into the genome of the cell. In other embodiments, the heterologous expression cassette is on a plasmid.

As disclosed herein, the genes of interest in present disclosure include, but not limited to, Uncoupling protein 1 (UCP1/SLC25A7), PPARG coactivator 1 alpha (PPARGC1A/PCG1A), PR/SET domain 16 (PRDM16), FFAR1/GPR40, FFAR4/GPR120, FOXO1, GLUT1, GLUT2, GLUT3, GLUT4, CHREBP, CD36, CTP1B, AIFM2/FSP1, GSK, IRS1, DGAT1, DGAT2, UPP1, ACACA, ACACB, CKB and NRIP1 and as found in Table 2.

### Methods of Treatment

In one aspect, the disclosure provides methods for preventing or treating a metabolic disease in a subject, the method comprising upregulating at least one polypeptide selected from the group consisting of Uncoupling protein 1 (UCP1/SLC25A7), PPARG coactivator 1 alpha (PPARGC1A/PCG1A), PR/SET domain 16 (PRDM16), FFAR1/GPR40, FFAR4/GPR120, FOXO1, GLUT1, GLUT2, GLUT3, GLUT4, CHREBP, CD36, CTP1B, AIFM2/FSP1, GSK, IRS1, DGAT1, DGAT2, UPP1, CKB, and ACACA or as found in Table 2, in a adipocyte of the subject, thereby preventing or treating a metabolic disease.

In another aspect, the disclosure provides methods for preventing or treating a metabolic disease in a subject, the method comprising downregulating at least one polypeptide selected from the group consisting of ACACB and NRIP1, in an adipocyte of the subject, thereby preventing or treating a metabolic disease.

As used herein, the term “metabolic disease” refers to a disease, disorder, or syndrome that is related to a subject's metabolism, such as breaking down carbohydrates, proteins, and fats in food to release energy, and converting chemicals into other substances and transporting them inside cells for energy utilization and/or storage. Some symptoms of a metabolic disease include high serum triglycerides, high low-density cholesterol (LDL), low high-density cholesterol (HDL), and/or high fasting insulin levels, elevated fasting plasma glucose, abdominal (central) obesity, and elevated blood pressure. Metabolic diseases increase the risk of developing other diseases, such as cardiovascular disease. Examples of metabolic diseases include, but are not limited to, obesity, Type-1 diabetes, and Type-2 diabetes.

In one aspect, the disclosure provides methods for preventing or treating diabetes in a subject, the method comprising upregulating at least one polypeptide selected from the group consisting of Uncoupling protein 1 (UCP1/SLC25A7), PPARG coactivator 1 alpha (PPARGC1A/PCG1A), PR/SET domain 16 (PRDM16), FFAR1/GPR40, FFAR4/GPR120, FOXO1, GLUT1, GLUT2, GLUT3, GLUT4, CHREBP, CD36, CTP1B, AIFM2/FSP1, GSK, IRS1, DGAT1, DGAT2, UPP1, CKB, and ACACA or as found in Table 2, in a adipocyte of the subject, thereby preventing or treating diabetes.

In another aspect, the disclosure provides methods for preventing or treating diabetes in a subject, the method comprising downregulating at least one polypeptide selected from the group consisting of ACACB and NRIP1, in an adipocyte of the subject, thereby preventing or treating diabetes

In one aspect, the disclosure provides methods for preventing or treating cancers in a subject, the method comprising upregulating at least one polypeptide selected from the group consisting of Uncoupling protein 1 (UCP1/SLC25A7), PPARG coactivator 1 alpha (PPARGC1A/PCG1A), PR/SET domain 16 (PRDM16), FFAR1/GPR40, FFAR4/GPR120, FOXO1, GLUT1, GLUT2, GLUT3, GLUT4, CHREBP, CD36, CTP1B, AIFM2/FSP1, GSK, IRS1, DGAT1, DGAT2, UPP1, CKB, and ACACA or as found in Table 2, in a adipocyte of the subject, thereby preventing or treating cancers.

In another aspect, the disclosure provides methods for preventing or treating cancers in a subject, the method comprising downregulating at least one polypeptide selected from the group consisting of ACACB and NRIP1, in an adipocyte of the subject, thereby preventing or treating cancers.

Examples of cancers that can be treated or prevented with the methods described herein include, but are not limited to, breast cancer, colon cancer, pancreas cancer, or prostate cancer.

In some embodiments, the method improves at least one symptom selected from the group consisting of body weight loss, cold tolerance, oxygen consumption, whole body energy expenditure, glucose and fatty acid utilization, glucose sensitivity, and insulin sensitivity. In some embodiments, the method results in suppression of glycolysis or fatty acid metabolism in cancer cells. In some embodiments, the method results in declining aging.

In some embodiments, the method improves at least one symptom of aging. Examples of symptoms of aging include but are not limited to skin damage, reduces muscle and physical strength, reduced sight and hearing and smell, changes in sleep patterns, changes in energy and appetite, reduction in memory and cognition, bone density loss, reduced muscle mass, blood vessels becoming stiffer that lead to increase in blood pressure, less elastic lungs, reduced immune function, smaller kidneys, menopause in women and reduced libido in men, reduction in hormone levels (such as growth hormone, aldosterone, insulin) and reduction in the production of red blood cells.

### Administration of Constructs

In some embodiments, the upregulation of the gene expression comprises introducing to the subject a therapeutically effective amount of a composition comprising a targeted polypeptide linked to a transcriptional activator, or a nucleic acid encoding the targeted polypeptide linked to a transcriptional activator, wherein the targeted polypeptide targets a portion of a promoter or enhancer sequence operably linked to a polynucleotide encoding the polypeptide.

In some embodiments, the upregulation of the gene expression comprises introducing to the subject a therapeutically effective amount of a composition comprising a heterologous expression cassette comprising a promoter operably linked to a coding sequence encoding the gene.

As non-limiting examples, the compositions or plurality thereof can be administered to the subject intravenously, intramuscularly, intrathecally, or a combination thereof. In some embodiments, the compositions are administered to a fat cell in the subject.

### Administration of Cells

In other embodiments, the methods can comprise introducing to the subject a therapeutically effective amount of a composition comprising engineered adipocytes or adipose stem cells comprising a targeted polypeptide linked to a transcriptional activator, wherein the targeted polypeptide targets a portion of a promoter or enhancer sequence operably linked to a polynucleotide encoding the polypeptide.

In certain embodiments, before introducing to the subject the engineered adipocytes or adipose stem cells, one or more adipocytes or adipose stem cells are isolated from the subject and engineered to have an upregulated expression of the gene of interest. The isolated cells (e.g., adipocytes, adipose stem cells, adipose progenitor cells) can undergo ex vivo gene therapy to upregulate the expression of the target gene, and the modified cells can be introduced into the human subject. Ex vivo gene therapy is a therapeutic approach that typically includes isolation and ex vivo expansion and/or manipulation of cells and subsequent administration of these cells to a patient. The isolated cells may be manipulated to overexpress or upregulate the expression of the target gene in any one of the known ways, including, for example, by using RNA and DNA transfection, viral transduction, or electroporation, for example. Thereby the engineered cells have an upregulated expression of a gene of interest by the introduced targeted polypeptide linked to a transcriptional activator, wherein the targeted polypeptide targets a portion of a promoter or enhancer sequence operably linked to a polynucleotide encoding the polypeptide. In some embodiments, the engineered adipocytes or adipose stem cells can be administered to the same subject (i.e., autologous cells). In other embodiments, the engineered adipocytes or adipose stem cells can be administered to a different subject (i.e., allogenic cells).

Any cell described herein (including organoids described below) can be provided in a biocompatible matrix, optionally comprising polycaprolactone. In some embodiments, the cells are delivered into an individual (e.g., a human), for example to achieve a therapeutic effect, and optionally at a later point can be removed from the individual. Examples of biocompatible matrices comprising polycaprolactone for delivery of cells are described in, e.g., US Patent Publication No. 2019/0119462.

### Organoids

In certain embodiments, the engineered adipocytes or adipose stem cells are cultured into an engineered adipose organoid, and the engineered adipose organoid is introduced into the subject to prevent or treat metabolic diseases, diabetes, or cancers. In some embodiments, the engineered adipose organoid can be administered to the same subject (i.e., autologous cells). In other embodiments, the engineered adipose organoid can be administered to a different subject (i.e., allogenic cells).

As non-limiting examples, the engineered adipocytes, adipose stem cells, or the engineered adipose organoid can be administered to the subject by targeted implantation, or intravenously, intramuscularly, intrathecally, or a combination thereof.

### Engineering Fat Cells to Consume a Metabolite Preferentially Consumed by Cancer Cells

In certain embodiments, adipocytes or adipose stem cells are engineered to consume a metabolite preferentially consumed by cancer cells compared to non-cancer cells in an individual. There are a number of types of cancer that preferentially (compared to healthy non-cancer cells in the individual) consume specific metabolites. It has been discovered that one can engineer adipocytes or adipose stem cells (or an organoid comprising such cells) to also consume the same metabolite and such cells can compete with the cancer cells for the metabolite, thereby reducing or preventing growth of the cancer in the individual.

A variety of cancer cells are known to preferentially consume certain metabolites. Examples of such cells and metabolites include, but are not limited to, pancreatic ductal adenocarcinoma or other cancer cells and uridine (see, e.g., Nwosu, Z. C. et al. Nature 618, 151-158 (2023)). Other metabolites used by cancer cells in general and that can be targeted by adipocytes or adipose stem cells engineered to consume the metabolite include those in the following Table 2. The metabolite or functional pathway is listed to the left under “Pathway” and examples of genes that can be expressed in the adipocytes, adipose organoids or adipose stem cells to metabolize the targeted metabolite or pathway are listed under “Function.”

TABLE 2

| Pathway | Gene | Function | PubMed # |
|---|---|---|---|
| Glycolysis | ALDOA | Fructose-bisphosphate aldolase | 34862480 |
| | ENO1 | Enolase | 34862480 |
| | FOXO1 | Transcription factor that is the main target of insulin | 32629884 |
| | | signaling | |
| | GAPDH | Glyceraldehyde 3-phosphate dehydrogenase | 34862480 |
| | Glucagon | Glucose levels | 34862480 |
| | GSK3B | A negative regulator in the hormonal control of glucose | 33989699 |
| | | homeostasis | |
| | GPI | Glucose phosphate isomerase | 34862480 |
| | Insulin | Hormone that regulates glucose levels | 34862480 |
| | HK1 | Hexokinase | 34862480 |
| | IRS1 | Mediates the control of various cellular processes by insulin | 22454254 |
| | MCT1 | Lactate influx | 34862480 |
| | MCT4 | Lactate eflux | 34862480 |
| | MLXIPL | Glucose sensing transcription factor | 34862480 |
| | LDHA | L-lactate dehydrogenase | 34862480 |
| | LDHB | L-lactate dehydrogenase | 34862480 |
| | PGK1 | Phosphoglycerate kinase | 34862480 |
| | PKLR | Pyruvate kinase | 34862480 |
| | PFKM | Phosphofructokinase | 34862480 |
| | SLC16A1 | Lactate-proton symporter | 34862480 |
| | SLC2A1/GLUT1 | Glucose transporter | 34862480 |
| | SLC2A2/GLUT2 | Glucose transporter | 34862480 |
| | SLC2A3/GLUT3 | Glucose transporter | 34862480 |
| | SLC2A4/GLUT4 | Glucose transporter | 34862480 |
| | TPI | Triosephosphate isomerase | 34862480 |
| Glutamine | ASCT2 | Glutamine transporter | 34862480 |
| | BCKDHA | Converts amino acids into ketoacids | 34862480 |
| | CAD | Carbamoyl-phosphate synthetase | 34862480 |
| | CTPS | CTP synthase | 34862480 |
| | GFPT1 | Fructose 6-phosphate transaminase | 34862480 |
| | GFPT2 | Fructose 6-phosphate transaminase | 34862480 |
| | GLS1 | Gluatminase | 34862480 |
| | GLS2 | Gluatminase | 34862480 |
| | GLUL | Glutamine synthesaze | 34862480 |
| | GMPS | Guanine monophosphate synthase | 34862480 |
| | GOT2 | Glutamic-oxaloacetic transaminase | 34862480 |
| | GPT2 | Glutamic-pyruvic transaminase | 34862480 |
| | KEAP1 | Activates NRF2 | 34862480 |
| | LAT1 | Transporter of glutamine efflux | 34862480 |
| | NADSYN | Glutamine-dependent NAD+ synthetase | 34862480 |
| | NRF2 | Transcription factor that induces expression of SLC7A11 | 34862480 |
| | PPAT | phosphoribosyl pyrophosphate amidotransferase | 34862480 |
| | PFAS | phosphoribosylformylglycinamidinesynthase | 34862480 |
| | SLC6A19 | Sodium-dependent neutral amino acid transporter | 34862480 |
| | SLC7A11 | Transporter of cystine/glutamate | 34862480 |
| | SNAT1 | Transporter of glutamine efflux | 34862480 |
| Fatty acid | ACC1 | Produces malonyl-CoA | 34862480 |
| | ACC2 | Produces malonyl-CoA | 34862480 |
| | ACSS2 | produces acetyl-CoA from acetate, | 34862480 |
| | CD36 | Glycoprotein that acts as a receptor for long-chain fatty | 35224665 |
| | | acids | |
| | CPT1A | Converts fatty acly-CoAs to fatty acyl-carnitines | 34862480 |
| | CPT1B | Rate-controlling enzyme of long-chain fatty acid beta- | 34862480 |
| | | oxidation | |
| | CPT2 | Converts fatty acyl-carnitines to fatty acly-CoAs | 34862480 |
| | DGAT1 | Fatty acid breakdown enzyme | 33413672 |
| | DGAT2 | Fatty acid breakdown enzyme | 33413672 |
| | FASN | Elongates malonyl-CoA | 34862480 |
| | FFAR1 | G-protein-coupled receptor for long-chain fatty acids | 27757756 |
| | FFAR4 | G-protein-coupled receptor for long-chain fatty acids | 27757756 |
| | SCD1 | oxygen-dependen iron-containing enzyme that produce | 34862480 |
| | | oleate | |
| | SLC25A20 | Transports fatty acyl-carnitines to mitochondria | 34862480 |
| Nucleotides | ADSL | Adenylosuccinate lyase | 34862480 |
| | AICAR | 5-aminoimidazole-4-carboxamide ribonucleotide | 34862480 |
| | ATIC | Bifunctional purine biosynthesis protein | 34862480 |
| | CAD | Carbamoyl-phosphate synthetase | 34862480 |
| | DHFR | Dihydrofolate reductase | 34862480 |
| | DHODH | Dihydroorotate dehydrogenase | 34862480 |
| | FOLR1 | Folate receptor | 34862480 |
| | IMPDH | Inosine monophosphate dehydrogenase | 34862480 |
| | MTHFD2 | Methylenetetrahydrofolate dehydrogenase 2 | 34862480 |
| | PHGDH | Phosphoglycerate dehydrogenase | 34862480 |
| | PRPS1 | Phosphoribosyl pyrophosphate synthetase | 34862480 |
| | PSAT1 | Phosphoserine aminotransferase 1 | 34862480 |
| | SHMT1 | Serine hydroxymethyl transferases | 34862480 |
| | SHMT2 | Serine hydroxymethyl transferases | 34862480 |
| | TYMS | Thymidylate synthetase | 34862480 |
| | UMPS | UMP synthase | 34862480 |
| Uridine | UPP1 | Uridine phosphorylase | 37198494 |
| Aspartate | ASS1 | Consumes aspartate to produce arginosuccinate from | 34862480 |
| | | citrulline | |

The adipocytes or adipose stem cells can be engineered to consume these metabolites, for example, but introducing or overexpressing one or more genes to improve consumption of the metabolite by the cells. For example, expression of uridine phosphorylase (UPP1) by the cells will increase consumption by the cells of uridine. Expression of these genes (for example, but not limited to, UPP1) can be increased in any number of ways, including but not limited to, use of CRISPRa increasing endogenous UPP1 expression, introduction of an expression cassette encoding UPP1 or editing the genome of the cell to overexpress UPP1.

The cells engineered to consume the metabolites can be introduced into an individual such that the cells are in proximity to the relevant cancers such that the cells compete with the cancer cells for the metabolite, thereby reducing or stopping growth of the cancer cells in the individual.

### Pharmaceutical Formulations

The compositions (e.g., expression cassettes, or cells having upregulated or overexpressed gene products) described herein can be formulated together with a pharmaceutically acceptable carrier. The compositions can additionally contain other therapeutic agents that are suitable for treating or preventing a given disorder. Pharmaceutically carriers can enhance or stabilize the composition, or to facilitate preparation of the composition. Pharmaceutically acceptable carriers include solvents, dispersion media, coatings, antibacterial and antifungal agents, isotonic and absorption delaying agents, and the like that are physiologically compatible.

A pharmaceutical composition as described herein can be administered by a variety of methods known in the art. The route and/or mode of administration vary depending upon the desired results. In some embodiments, the composition is sterile and fluid. Proper fluidity can be maintained, for example, by use of coating such as lecithin, by maintenance of required particle size in the case of dispersion and by use of surfactants. In many cases, it is preferable to include isotonic agents, for example, sugars, polyalcohol such as mannitol or sorbitol, and sodium chloride in the composition. Long-term absorption of the injectable compositions can be brought about by including in the composition an agent which delays absorption, for example, aluminum monostearate or gelatin.

Pharmaceutical compositions described herein can be prepared in accordance with methods well known and routinely practiced in the art. Pharmaceutically acceptable carriers are determined in part by the particular composition being administered, as well as by the particular method used to administer the composition. Accordingly, there is a wide variety of suitable formulations of pharmaceutical compositions. Applicable methods for formulating the compositions and determining appropriate dosing and scheduling can be found, for example, in Remington: The Science and Practice of Pharmacy, 21^{st} Ed., University of the Sciences in Philadelphia, Eds., Lippincott Williams & Wilkins (2005); and in Martindale: The Complete Drug Reference, Sweetman, 2005, London: Pharmaceutical Press., and in Martindale, Martindale: The Extra Pharmacopoeia, 31st Edition., 1996, Amer Pharmaceutical Assn, and Sustained and Controlled Release Drug Delivery Systems, J. R. Robinson, ed., Marcel Dekker, Inc., New York, 1978, each of which are hereby incorporated herein by reference. Pharmaceutical compositions are preferably manufactured under GMP conditions. Typically, a therapeutically effective dose or efficacious dose is employed in the pharmaceutical compositions described herein. The compositions can be formulated into pharmaceutically acceptable dosage forms. Dosage regimens are adjusted to provide the desired response (e.g., a therapeutic response). In determining a therapeutically or prophylactically effective dose, a low dose can be administered and then incrementally increased until a desired response is achieved with minimal or no undesired side effects. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients in the pharmaceutical compositions can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

### EXAMPLES

Many modifications and variations of this invention can be made without departing from its spirit and scope, as will be apparent to those skilled in the art. The specific embodiments described herein are offered by way of example only and are not meant to be limiting in any way.

All documents (for example, patents, patent applications, books, journal articles, or other publications) cited herein are incorporated by reference in their entirety and for all purposes, to the same extent as if each individual document was specifically and individually indicated to be incorporated by reference in its entirety for all purposes. To the extent such documents incorporated by reference contradict the disclosure contained in the specification, the specification is intended to supersede and/or take precedence over any contradictory material.

### Example 1

Here, we set out to develop a novel cancer therapeutic approach, termed adipose manipulation transplantation (AMT) that utilizes two unique abilities of white adipose tissue (WAT): 1) They can be readily extracted via liposuction and implanted via reconstructive surgery; 2) they can change into a BAT-like tissue, called browning or beiging (16, 17), by upregulating essential transcriptional regulators or enzymes, such as the uncoupling protein 1 (UCP1), PPARG coactivator 1 alpha (PPARGC1A) or PR/SET domain 16 (PRDM16), involved in BAT development and function (18-33). Similar to BAT, beige adipocytes have the capacity to convert energy to heat and contribute to whole-body energy expenditure (34). We show that CRISPR activation (CRISPRa) of either UCP1, PRDM16 or PPARGC1A, induces browning which subsequently increases glucose and fat metabolism in human white adipocytes and adipocyte organoids. Co-culturing of these CRISPRa-modulated adipocytes with various cancer cell lines (breast, colon, pancreatic, or prostate cancer) leads to significantly suppressed cancer cell proliferation as well as decreased glucose uptake, glycolysis and fatty acid oxidation capacity in the cancer cells. Subcutaneously co-transplanting CRISPRa-modulated human adipose organoids and cancer cells (breast, colon, pancreatic, or prostate) in immune-compromised mice leads to significantly reduced tumor size with decreased hypoxia and angiogenesis. Implantation of engineered adipose organoids in pancreatic or breast cancer genetic mouse models significantly reduces cancer progression. Furthermore, in the breast cancer model we show that their implantation both near and distal to the tumor leads to similar results. To further demonstrate the therapeutic potential of this approach, we show that adipocytes isolated from human dissected breast tissues can be similarly manipulated via CRISPRa and inhibit growth of patient-derived breast cancer organoids. Combined, our results introduce a novel cancer therapeutic approach that has the potential to treat numerous cancer types.

### Results

### CRISPRa Browning of Human White Adipocytes

To induce browning in human adipocytes we utilized CRISPRa to upregulate UCP1, PPARGC1A or PRDM16, all known genes involved in BAT development and function. Using CRISPick (35), we designed five gRNAs targeting each gene's promoter and cloned them into an adeno associated virus (AAV) based expression vector. Differentiated adipocytes derived from human white preadipocytes were co-transfected with the gRNAs along with a Staphylococcus aureus (sa) endonuclease deficient Cas9 (dCas9) fused to the VP64 transcriptional activator. We used an sa dCas9 due to its smaller size and the VP64 transcriptional activator, which carries four copies of VP16, a herpes simplex virus type 1 transcriptional activator (36), as it provides moderate gene upregulation and is small enough to fit into AAV, which has a 4.7 kilobase optimal packaging capacity (37). To generate mature human adipocytes, preadipocytes were subjected to adipocyte differentiation using a cocktail of 3-isobutyl-1-methylxanthine (IBMX), dexamethasone, and insulin before being subjected to CRISPRa. Following four days, we measured gene expression via qRT-PCR, finding several gRNAs to significantly increase the expression levels of UCP1, PPARGC1A, PRDM16 in the transfected cells compared to the control cells (treated with dCas9-VP64 only) (FIG. 11A). We selected the top two gRNA for each gene for AAV serotype 9 (AAV9) packaging. We used AAV9, as it was shown to effectively infect various adipose depots (38). We infected human differentiated adipocytes with these viruses along with AAV9 dCas9-VP64, finding for each gene at least one gRNA that significantly increased expression levels compared to dCas9-VP64 only control (FIG. 11B). We used the top activating gRNA AAV for all subsequent experiments.

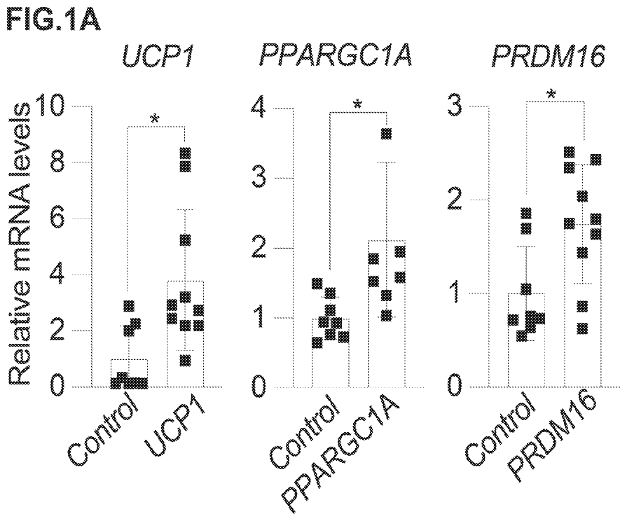

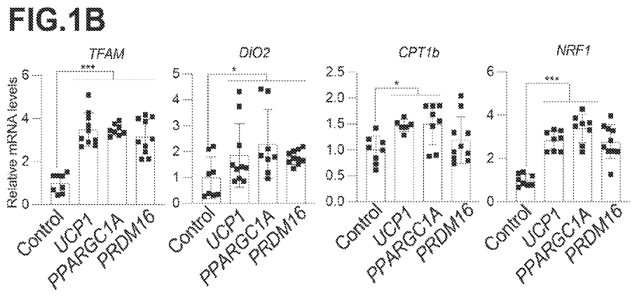

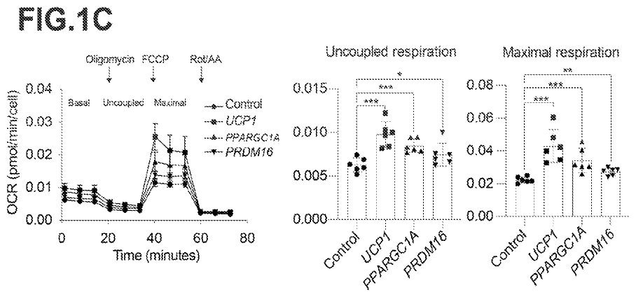

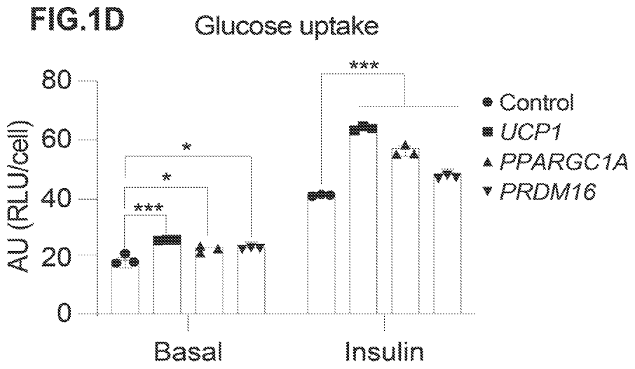

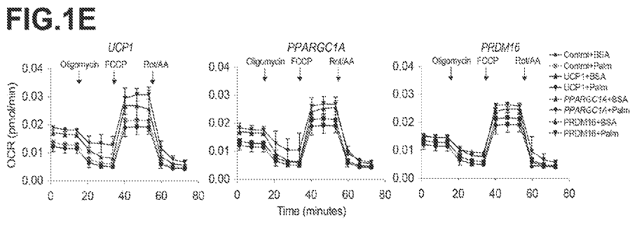

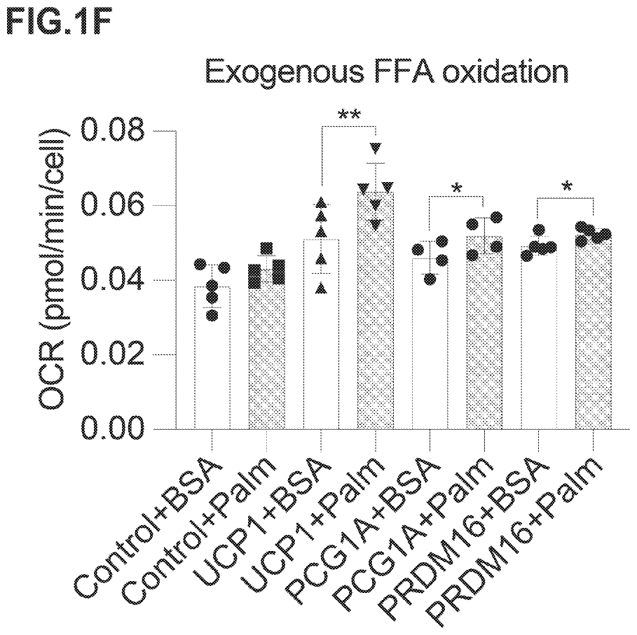

We next examined whether our CRISPRa treatment increases browning in these human white adipocytes. Human adipocytes infected with the top gRNA for UCP1, PRDM16 or PPARGC1A showed significant upregulation of their target genes compared to dCas9-VP64 only infected cells (FIG. 1A). In addition, we also observed increased mRNA levels for brown fat marker genes, including TFAM, DIO2, CTP1b, and NRF1 upon upregulation of either of the three genes (FIG. 1B; PRDM16 CRISPRa did not show upregulation of CTP1b, and NRF1). We examined oxygen consumption rate (OCR) in these cells using Seahorse (see Methods), by initially blocking oxygen consumption and then adding 1 μM oligomycin, followed by the introduction of 1 μM FCCP to measure the maximal respiratory capacity. We found that CRISPRa-AAV treatment targeting either gene increased overall OCR levels in human white adipocytes with UCP1 gRNA AAV treated cells having the largest increase (FIG. 1C). In addition, these cells showed increased uncoupled respiration (under oligomycin, ATPase inhibitor) indicating a brown fat-like phenotype (FIG. 1C). CRISPRa treated cells also had elevated maximal respiration following carbonyl cyanide-p-trifluoromethoxy-phenylhydrazone (FCCP) treatment (FIG. 1C). Furthermore, these AAV-CRISPRa engineering adipocytes showed increased glucose uptake in both basal and insulin stimulated conditions (FIG. 1D). We also tested the fatty acid oxidation (FAO) capacity of these cells by performing a similar Seahorse OCR assay. In both BSA- and BSA conjugated-palmitate (saturated fatty acid complex) media, we found that our CRISPRa-treated adipocytes had an overall OCR increase in BSA conjugated-palmitate (FIG. 1E). Under FCCP treatment, in which the increase of OCR in palmitate-containing media compared to BSA-containing media is thought to be due to exogenous fatty acid oxidation, we found that upregulation of either gene increased exogenous FAO capacity in human adipocytes (FIG. 1F). Taken together, we show that AAV-based CRISPRa upregulation of UCP1, PPARGC1A or PRDM16 induces browning in human adipocytes, leading them to have increased glucose uptake and fatty acid oxidation.

### CRISPRa-Modulated Adipocytes Suppress Cancer Cell Growth In Vitro

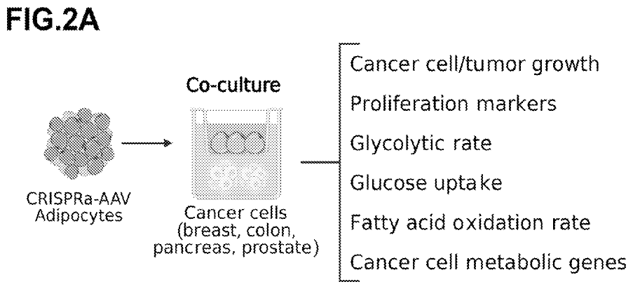

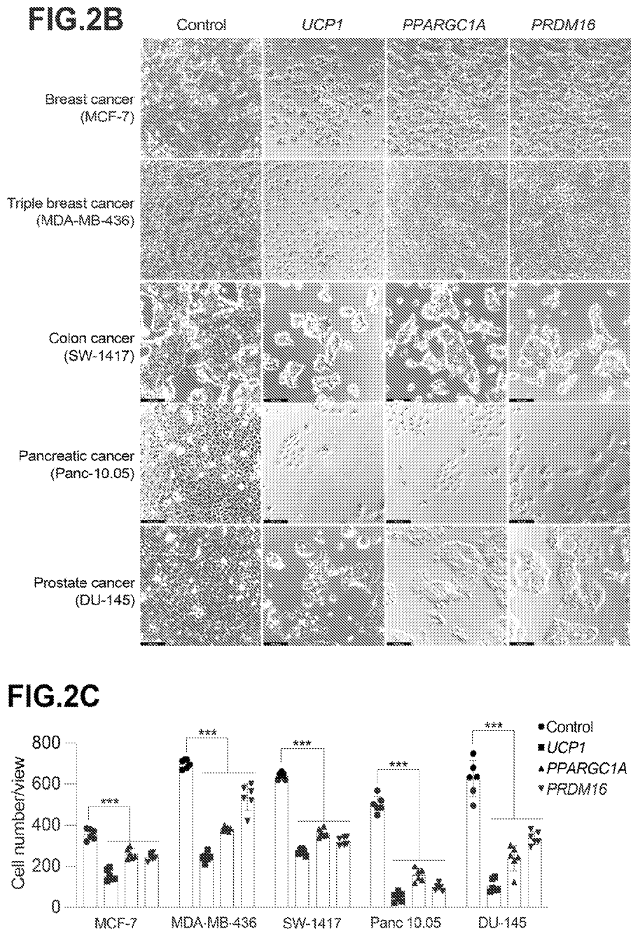

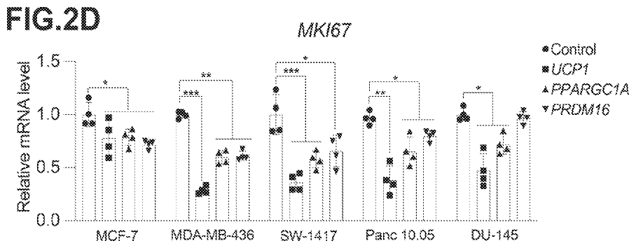

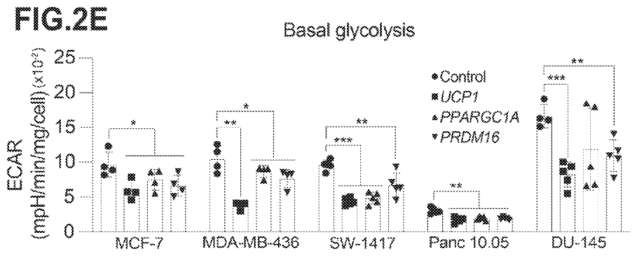

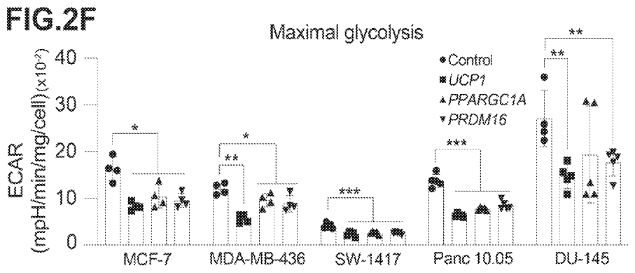

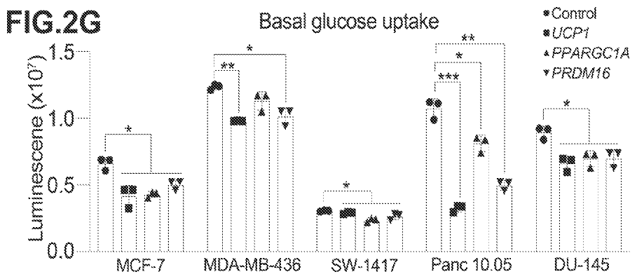

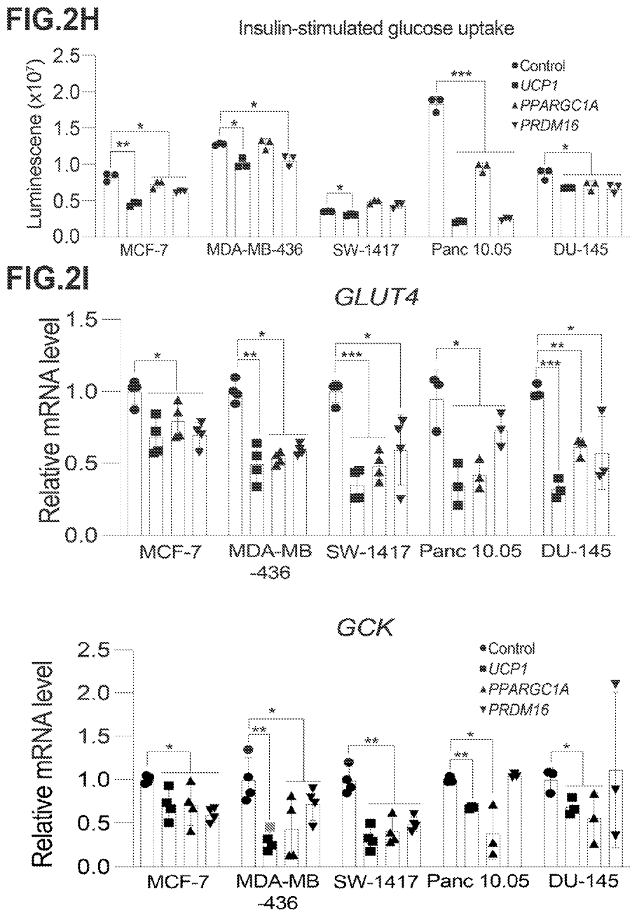

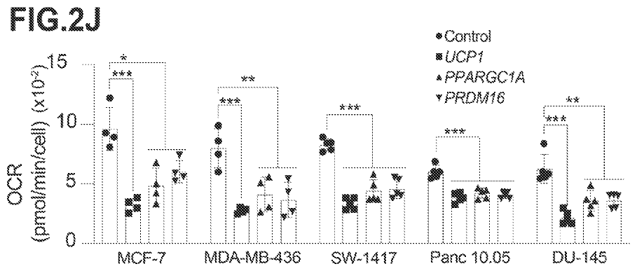

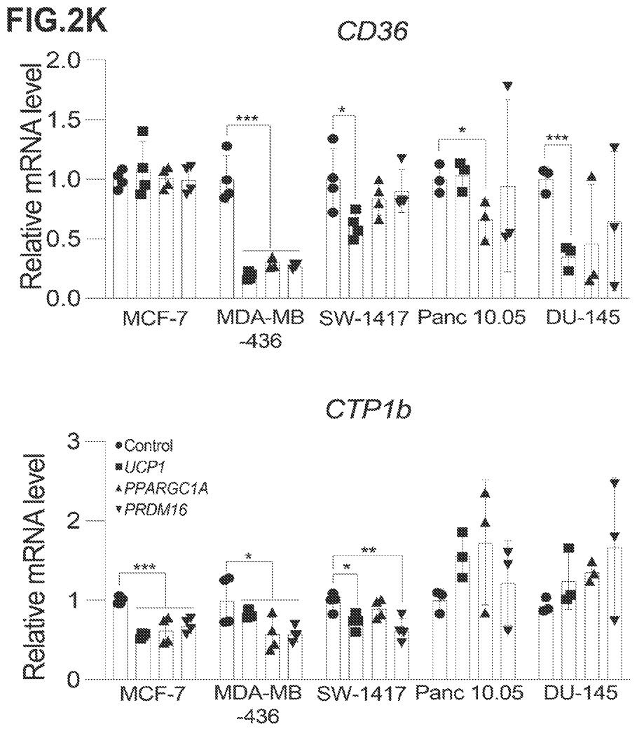

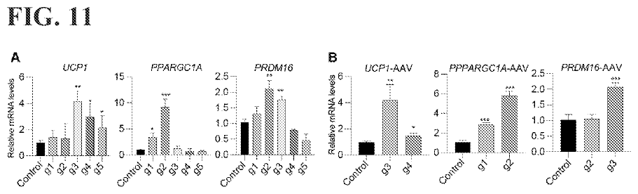

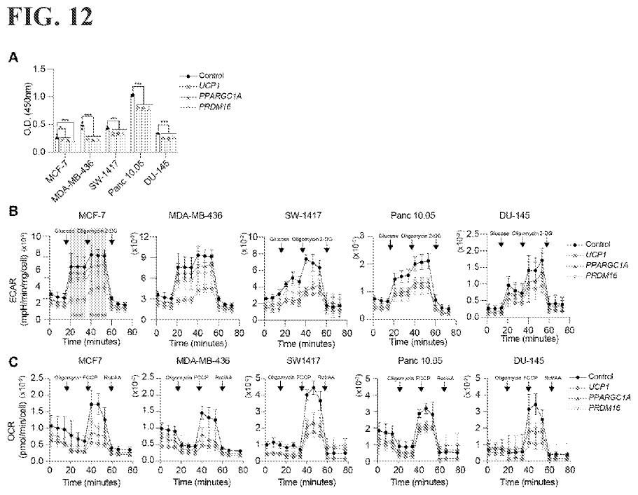

We next evaluated whether our CRISPRa ‘browned’ adipocytes could inhibit cancer growth in vitro using a co-culturing system (FIG. 2A). We initially treated differentiated adipocytes with CRISPRa-AAV for either of the three genes (UCP1, PPARGC1A or PRDM16) and replated these adipocytes on the top chamber of a 12- or 24-well transwell plate, which has inserts of 0.4 μm membrane, so that the adipocytes don't contact the cells on the lower chamber (FIG. 2A). In the lower chamber, we grew five different cancer cell lines: breast cancer cells MCF7 (ER+,PR+,GR+) and MDA-MB-436 (triple negative), colon cancer (SW-1417), pancreatic cancer (Panc 10.05) and prostate cancer (DU-145). As a negative control, we used adipocytes infected with dCas9-VP64 only. After three days, we observed that all five cancer cell lines that were co-cultured with UCP1, PPARGC1A or PRDM16 CRISPRa-treated human adipocytes showed significantly lower cell numbers than cells co-cultured with dCas9-VP64 treated adipocytes (FIG. 2B). We found that the number of cancer cells co-cultured with CRISPRa-treated adipocytes were 3- to 5-fold lower than cells cocultured with the control adipocytes (FIG. 2C). By qRT-PCR, we observed that all cancer cells co-cultured with CRISPRa-treated adipocytes had significantly decreased levels of MKI67 (other than DU-145 treated with PRDM16 CRISPRa) a proliferation marker, compared to control cells, with UCP1 CRISPRa having the greatest effect (FIG. 2D). We also performed a BrdU incorporation assay, finding that after 24 hours the CRISPRa co-cultured cancer cells showed significantly reduced proliferation compared to cells co-cultured with control adipocytes (FIG. 12A).

We next analyzed the glucose and fatty acid metabolism of the cancer cells. To measure glycolysis, we utilized the extracellular acidification rate (ECAR) assay in which the basal glycolytic rate is measured by adding glucose and the maximal glycolytic rate is measured by oligomycin A addition. We found that all five cancer cell lines co-cultured with CRISPRa-AAV treated adipocytes showed lower overall glycolysis than those co-cultured with dCas9-VP64 only (FIG. 12B). These cells also showed a significant reduction in both basal and maximal glycolytic rate (FIG. 2E-F) and lower glucose uptake in both basal and insulin conditions (FIG. 2G-H), other than SW-1417 treated with either PPARGC1A or PRDM16 CRISPRa. Using qRT-PCR, we also found that the expression of key glycolysis genes, such as GCK and GLUT4, a major glucose transporter, were significantly lower in the cancer cells co-cultured with CRISPRa-AAV adipocytes (other than GCK for PRDM16 CRISPRa treated Panc 10.05 and DU-145 cells) compared to the negative control (FIG. 21). We also examined fatty acid oxidation (FAO) using Seahorse. Similar to glycolysis, we found that all five cancer cell lines that were co-cultured with CRISPRa-AAV adipocytes had reduced FAO compared to the negative control (FIG. 2J, FIG. 12C). Moreover, using qRT-PCR, we found in most CRISPRa conditions that the cancer cells have lower expression of both CPT1b, a key regulator of FAO in the mitochondria and CD36, a fatty acid transporter on the cell membrane, further confirming decreased FAO (FIG. 2J). Combined, our data shows that our CRISPRa-modulated adipocytes reduce glycolysis and fatty acid metabolism in five different cancer cell lines and can significantly suppress cancer growth.

### CRISPRa-Modulated Human Adipose Organoids Suppress Xenograft Growth

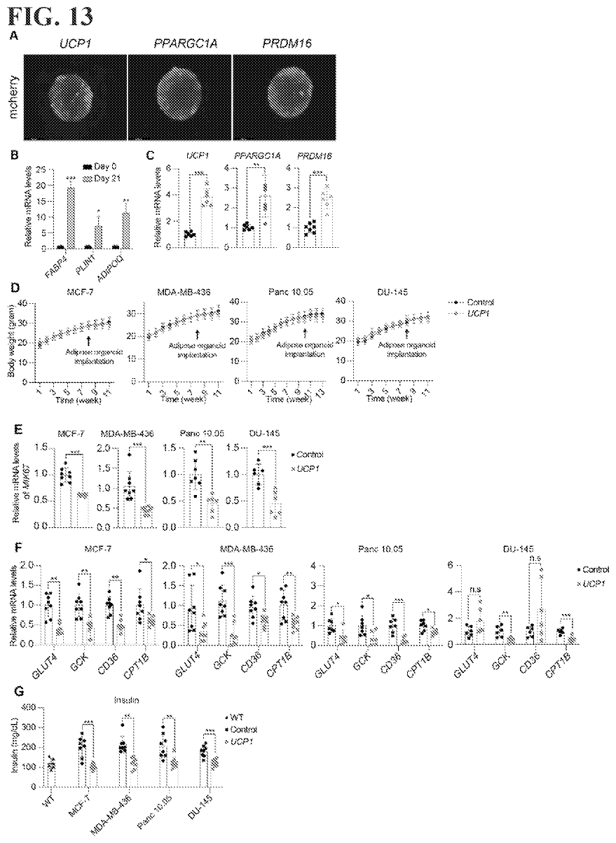

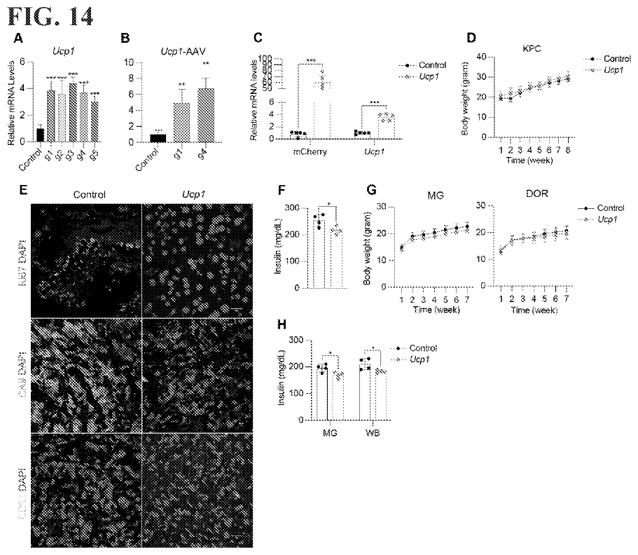

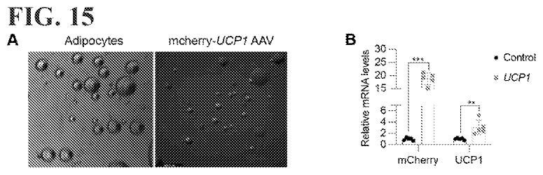

To examine whether CRISPRa-modulated adipocytes could inhibit cancer growth in in vivo xenograft models, we co-transplanted four cancer cell lines (MCF7 and MDA-MB-436 (breast), Panc 10.05 (pancreas) and DU-145 (prostate)) with CRISPRa-modulated human adipose organoids. While adipocytes could be used for co-transplantation, adipose organoids offer added advantages, including: 1) providing a 3D-culture that can better recapitulate the heterogeneity of adipose tissue; 2) enhanced response to endogenous stimuli; 3) the ability to form tissue microenvironments that could better integrate with cancer cells following transplantation. We initially established culturing conditions for human adipose organoids. We utilized immortalized human preadipocytes grown using methods that combines features from three different 3D culturing protocols (39, 40). In brief, we cultured human preadipocytes in basal DMEM media supplemented with 10% fetal bovine serum in Nunc 96 well plates treated with Nunclon Delta. Organoids formed after 48 hours and were then differentiated to adipose organoids using a differentiation cocktail containing IBMX, dexamethasone, insulin, T3, and rosiglitazone. Adipocytes formed 21-days post differentiation (FIG. 13A). Using qRT-PCR, we analyzed these organoids for various adipogenic markers, including FABP4, PLIN1, and ADIPOQ, finding all to be expressed (FIG. 13B). We further tested our ability to upregulate UCP1, PPARGC1A, and PRDM16 in these organoids using similar methods and AAV gRNAs as in the adipocytes, finding all three to show significant upregulation of the target genes (FIG. 13C). With the establishment of these adipose organoid culturing and CRISPRa conditions, we next set out to test whether they can suppress xenograft cancer growth.

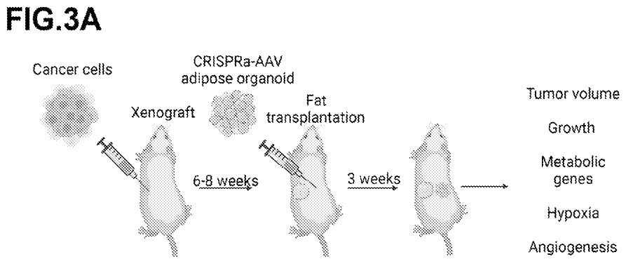

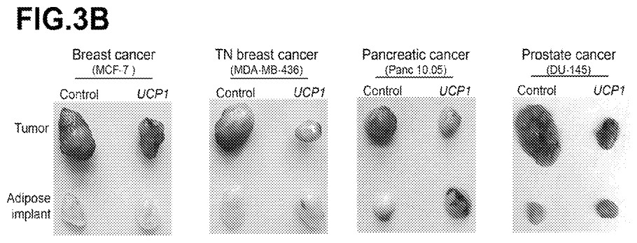

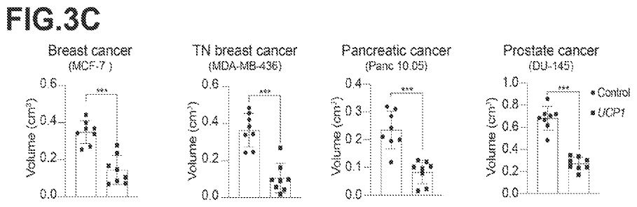

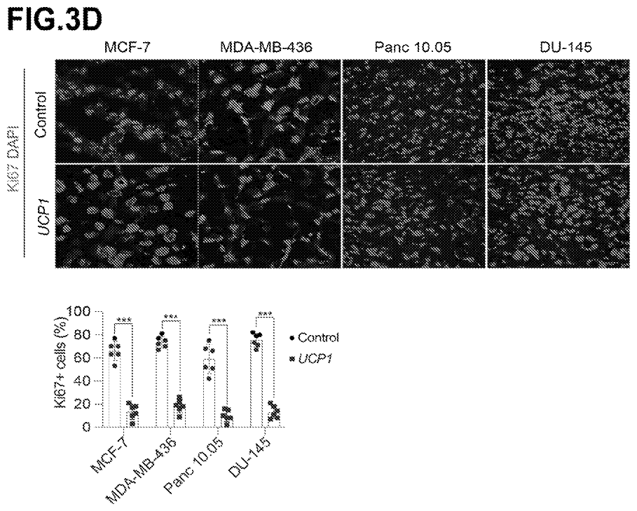

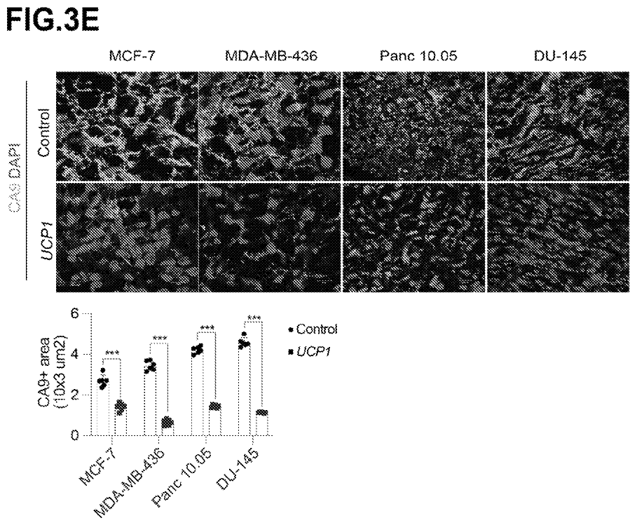

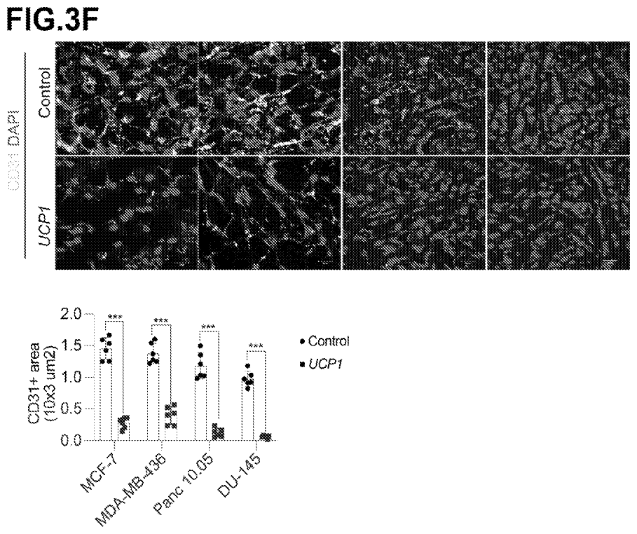

To generate xenografts, cancer cells were subcutaneously implanted into immuno-compromised SCID mice. Following 6-8 weeks, UCP1-CRISPRa treated human adipose organoids were mixed with Matrigel and co-transplanted adjacent to palpable tumors. For all subsequent assays, we only used UCP1-CRISPRa, as it showed the most optimal results in our cell culture experiments. Due to some reports suggesting that brown fat could be linked to cancer-associated cachexia (loss of skeletal muscle and fat) (41), we measured on a weekly basis the body weight of mice co-transplanted with cancer cells and UCP1-modulated adipose organoids, finding no significant differences between CRISPRa treated and control mice (FIG. 14D). Tumors and human adipose organoids were collected following three weeks (FIG. 3A). All tumor types co-transplanted with CRISPRa-modulated human adipose organoids were significantly smaller than dCas9-VP64 only transplanted human adipose organoids (FIG. 3B), having over 50% reduction in volume (FIG. 3C). Gene analysis showed that tumors co-transplanted with CRISPRa-treated adipose organoids had decreased expression of the proliferation marker gene MIK67 (FIG. 13E), as well as reduced marker gene expression for glycolysis (GLUT4, GCK) and fatty acid oxidation (CD36, CPT1B) (FIG. 13F). Using immunofluorescence, we examined additional tumor marker genes, finding that all tumors co-transplanted with UCP1-CRISPRa modulated human adipose organoids had markedly reduced Ki67+ (FIG. 3D). In addition, cancer cells showed decreased levels of hypoxia, identified by having a lower carbonic anhydrase (CA9+) area per image view (FIG. 3E). These tumors also showed decreased levels of CD31+ area per image view, indicating decreased microvessel density and suggesting a corresponding lower metastatic potential (FIG. 3F). Since brown fat is known to reduce insulin levels, we examined the plasma insulin in these mice. We found that plasma insulin levels of mice that were co-transplanted with UCP1-CRISPRa adipose organoids and cancers were significantly lower than those co-transplanted with the control adipose organoids and were comparable to levels in wild type SCID mice (FIG. 13G). Taken together, these results show that UCP1-CRISPRa modulated human adipose organoids significantly reduce glycolysis and fatty acid oxidation, reduce hypoxia and insulin levels and inhibit tumor growth for four different cancer types in vivo.

### AMT Suppresses Cancer Development in Pancreatic and Breast Cancer Genetic Mouse Models

To examine whether our AMT approach can prevent cancer development, we utilized pancreatic and breast cancer genetic mouse models. For pancreatic cancer, we used the KPC mouse model, that upon tamoxifen treatment develops pancreatic ductal adenocarcinoma due to conditional mutations in Kras and Trp53 (42). For breast cancer, we used MMTV-PyMT mice on an FVB background, containing a mouse mammary tumor virus (MMTV) long terminal repeat upstream of the Polyoma Virus middle T antigen (PyVmT), that develop mammary tumors with a mean latency of 53 days (43). We first designed five gRNAs targeting mouse Ucp1 and transfected them into mouse 3T3-L1 adipocytes, finding all five upregulate Ucp1 (FIG. 14A). We next selected the top two gRNA for AAV serotype 9 (AAV9) packaging and infected 3T3-L1 differentiated adipocytes with these viruses along with AAV9 dCas9-VP64, finding that all of them significantly increased Ucp1 gene expression levels compared to dCas9-VP64 only control (FIG. 14B). We used the top activating gRNA AAV9 for all subsequent experiments. As organoids have several of the aforementioned advantages, we generated adipose organoids using mouse preadipocytes, utilizing similar techniques as described for human adipose organoids (see Methods). We then infected them with AAV9 dCas9-VP64 and Ucp1-gRNA and observed both mCherry expression from our gRNA virus and significant Ucp1 upregulation (FIG. 14C).

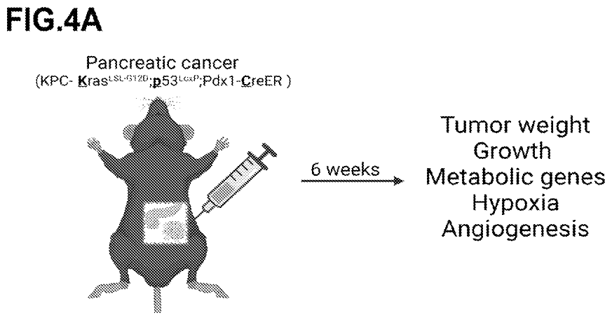

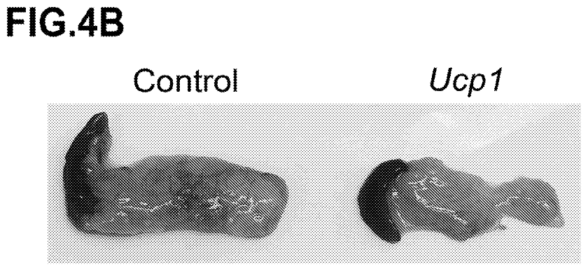

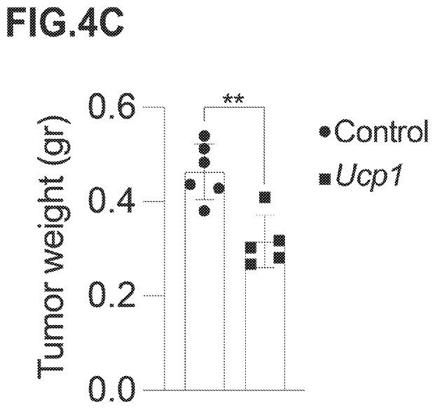

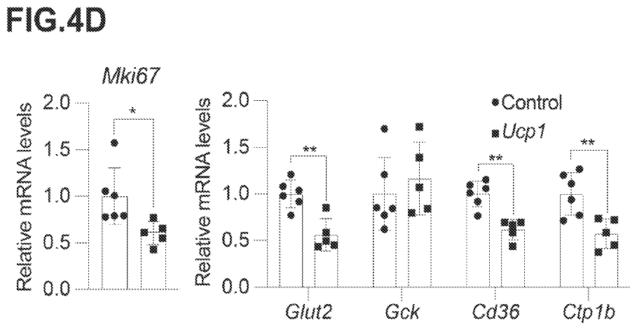

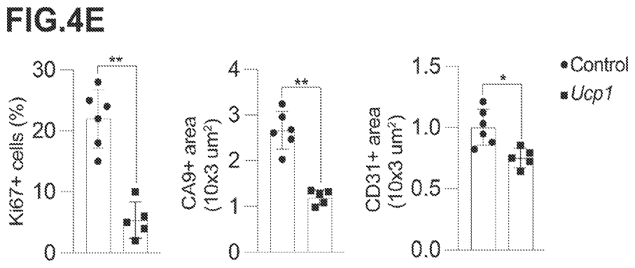

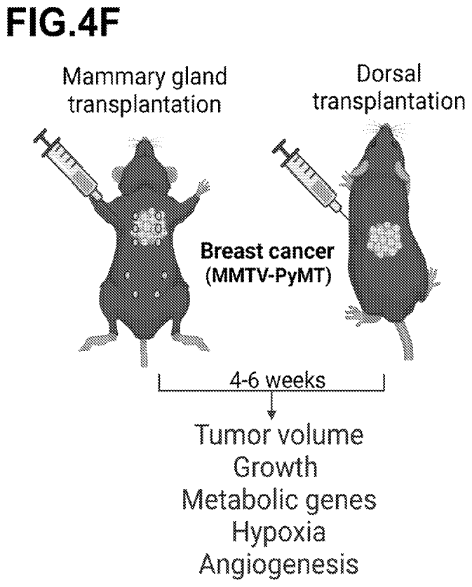

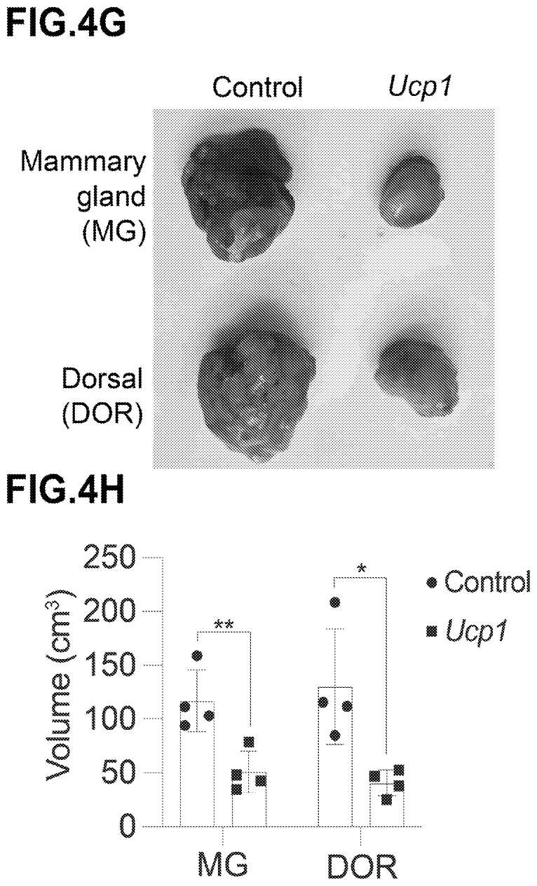

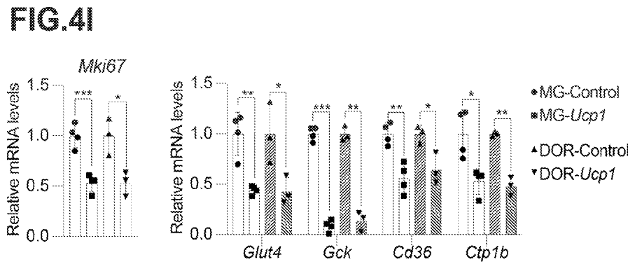

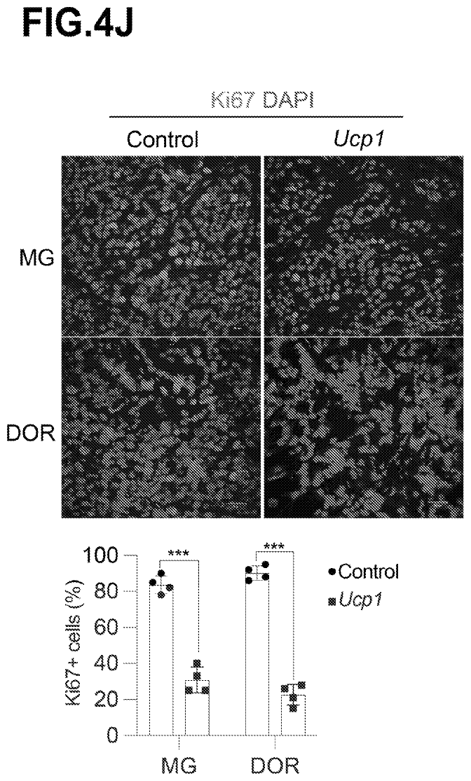

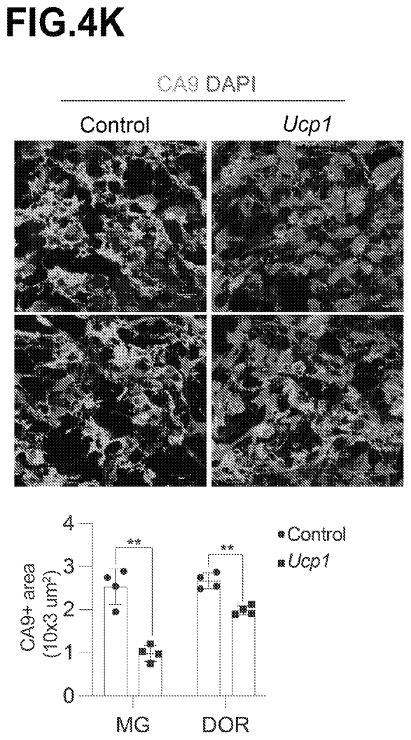

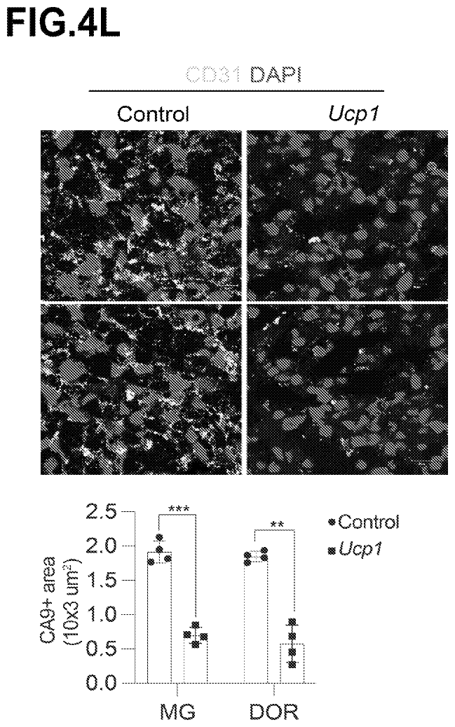

We first set out to test whether we can suppress pancreatic cancer development using KPC mice. KPC mice were treated with tamoxifen postnatal day 0-4. At four weeks of age, Ucp1-CRISPRa or dCas9-VP64 only (negative control) adipose organoids were orthotopically implanted next to the pancreas (FIG. 4A). Following six weeks, pancreases were removed and analyzed. Over these six weeks, we observed no significant difference in body weight between mice implanted with Ucp1-CRISPRa organoids or control organoids (dCas9-VP64 only) (FIG. 14D). We found that KPC mice implanted with Ucp1-CRISPRa upregulated adipose organoids had significantly smaller tumors compared to control dCas9-VP64 implanted mice (FIG. 4B) and reduced pancreatic mass (FIG. 4C). They also showed reduced expression of the proliferation marker, Mki67, as well as genes involved fatty acid oxidation (Cd36, Cpt1b) (FIG. 4D). In addition, we found Ucp1-CRISPRa mice to have lower expression levels of the pancreas' main glucose transporter, Glut2 (FIG. 4D). Ucp1-CRISPRa mice also had a lower number of Ki67+ cells compared to control mice as determined by immunofluorescence (FIG. 4E, 14E). Similarly, we observed lower CA9+ area per image, and CD31+ area per image view, suggesting reduced hypoxia and angiogenesis (FIG. 4E, 14E). In addition, we found Ucp1-CRISPRa adipose organoids to have significantly reduced insulin levels compared to control mice (FIG. 14F).

To examine whether our treatment might provide systematic therapeutic effect in suppressing breast cancer growth, we next used MMTV-PyMT female mice, implanting Ucp1-CRISPRa or dCas9-VP64 adipose organoids near the third nipples (MG) of four-week-old mice (FIG. 4F). As cold treatment leads to widespread BAT activation causing cancer suppression (15), we also examined whether distal implantation of adipose organoids could suppress cancer development by implanting organoids in the back of 4-week-old mice (DOR). Following six weeks post implantation, tumors were collected and analyzed. Similarly, we found no difference in body weight of mice implanted with Ucp1-CRIPSRa organoids compared to those implanted with control organoids (FIG. 14G). Remarkably, we found both strategies of organoid implantation resulted in significantly reduced tumor size (FIG. 4G) and volume (FIG. 4H) regardless of site of implantation. Tumors also had decreased expression of the Mki67 proliferation marker and metabolic genes, including Glut4, Gck, Cd36, and Cpt1b (FIG. 4I). The tumors of mice implanted with Ucp1-CRISPRa adipose organoids had less Ki67+ cells than control mice (FIG. 4J). These tumors also had lower CA9+ area per image, and CD31+ area per image view, indicating reduced hypoxia and angiogenesis (FIG. 4I-K). Additionally, mice implanted with Ucp1-CRISPRa adipose organoids had significantly lower plasma insulin levels than control mice (FIG. 14H). Taken together, our results indicate that our CRISPRa-modulated adipose organoids could have systematic therapeutic effects in suppressing cancer growth.

UCP1-CRISPRa Upregulation of Adipocytes from Dissected Breast Tumors Suppresses Cancer Growth.

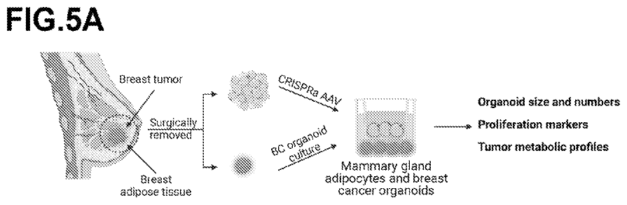

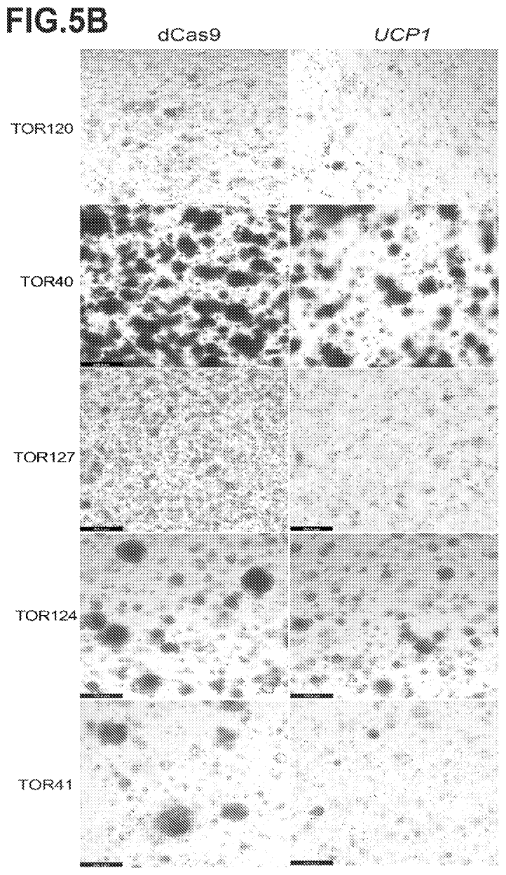

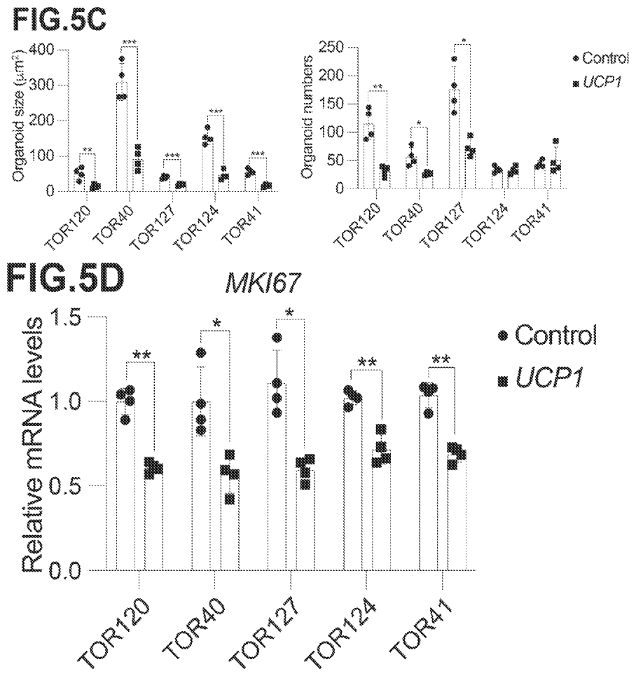

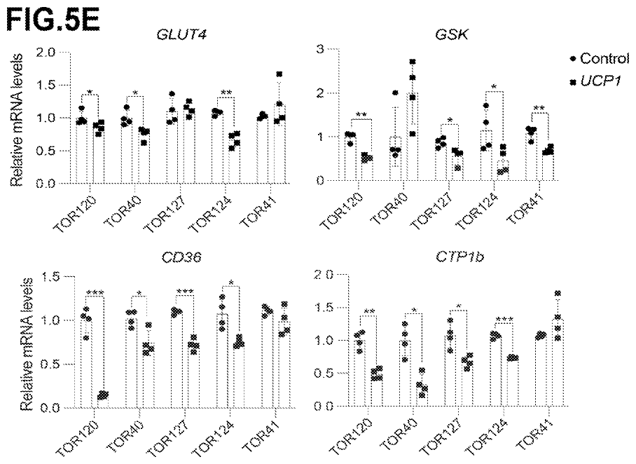

To further demonstrate the therapeutic potential of AMT, we treated adipocytes obtained from dissected human breast tissues with UCP1-CRIPSPRa AAV9 and tested their ability to suppress tumor progression by co-culturing them with breast cancer organoids generated from dissected breast tumors or metastatic pleural effusions (FIG. 5A). Samples were obtained from patients who underwent breast surgery or thoracentesis. For the co-culture experiments, we used five different breast cancer organoids generated from patients with early-stage or metastatic triple-negative breast cancer [estrogen receptor (ER)-negative, progesterone receptor (PR)-negative/human epidermal growth factor receptor 2 (HER2)-negative] or hormone receptor-positive (ER-positive and/or PR-positive)/HER2-negative breast cancer (Table S1). Organoids from breast tumor tissue were generated by digesting cells for one hour in collagenase and organoids from metastatic pleural effusions were generated by isolating tumor spheroids via centrifugation, before in both cases embedding cancer cells in organoid culture using established protocols that enable long-term propagation of tumor organoids (44). In parallel, primary human adipocytes were isolated from human breast tissue using an established protocol (45). For the triple-negative breast cancer organoids derived from primary tumors, we used two different cases, including one where we generated organoids and isolated mammary gland adipose tissue from the same individual (TOR41). Adipocytes were infected with dCas9-VP64 only (negative control) or UCP1-CRISPRa AAV9. After five days, they showed strong mCherry expression, a fluorescent marker that is part of the gRNA AAV9 (FIG. S5A), suggesting that they can be readily infected by our AAVs. In addition, gene analysis showed that both mCherry and UCP1 expression levels were significantly higher in the UCP1-CRISPRa treated adipose organoids compared to the control organoids (FIG. S5B).

TABLE S1
Breast cancer organoid status.

| Name | ER | PR | HER2 | Metastatic | Inflammatory? | Source of adipocytes? |
|---|---|---|---|---|---|---|
| TOR40 | Negative | Negative | Negative | no | yes | ad-1 (unmatched breast) |
| TOR120 | Negative | Negative | Negative | yes | no | ad-2 (unmatched breast) |
| TOR127 | Positive | Positive | Negative | yes | no | ad-3 (unmatched breast) |
| TOR124 | Negative | Negative | Negative | yes | no | ad-4 (unmatched breast) |
| TOR41 | Negative | Negative | Negative | no | yes | ad-5 (matched breast) |

To test whether UCP1-CRISPRa AAV9 adipocytes can suppress cancer growth, we added them to a hydrogel dome on top of the breast cancer organoids with a fully-defined organoid medium (44) and co-cultured for five days (FIG. 5A). In all five cases, we observed significant reduction in cancer organoid size (FIG. 5B) and in most organoids also a reduction in organoid number (TOR124 and TOR41 were not significant) upon incubation with adipocytes infected with UCP1-CRISPRa AAV9 compared to the dCas9-VP64 only treated cells (FIG. 5C). All five cancer organoid cases co-cultured with UCP1-CRISPRa had significantly lower proliferation marker MKI67 expression compared to negative control (FIG. 5D). We also observed significantly decreased levels of GLUT4, GCK, CD36, and CTP1b for the UCP1-CRISPRa co-cultured cancer organoids (FIG. 5E), suggesting they have lower glycolysis and fatty acid metabolism. Overall, our results demonstrate that human adipocytes from a dissected breast tissue can upregulate UCP1 via CRISPRa AAV9 and are able to reduce glycolysis and fatty acid metabolism and suppress breast cancer organoid growth. In addition, using matched mammary gland adipose tissue and breast cancer samples, we demonstrate the potential clinical utility of an ex vivo autologous transplantation of CRISPRa-modulated adipocytes to treat cancer.

### DISCUSSION

Cancer cells are fast-proliferating cells that require large amounts of nutrients, including glucose and fatty acids. They can reprogram metabolic pathways to utilize available substrates in the surrounding environment. Targeting their metabolism can be a potent cancer treatment. Here, we developed CRISPRa-modulated human adipocytes that have increased glucose and fatty acid utilization by upregulating UCP1, PPARGC1A, and PRDM16. These CRISPRa-modulated adipocytes were able to suppress cancer growth in five different cancer cells lines, including two breast lines, colon, pancreas, and prostate. Co-transplanting CRISPRa-treated human adipose organoids and xenografts in mice led to a significant reduction in tumor growth in all five cancer types. These tumors had lower glycolysis and fatty acid metabolism and showed decreased hypoxia and angiogenesis. Implanting CRISPRa-modulated adipose organoids into genetic mouse models of breast and pancreatic cancers also prevented tumor development in mice. Furthermore, we demonstrated that implantation of CRISPRa-modulated adipose organoids distant from the cancer tissue leads to similar results. In addition, co-culturing cancer organoids from breast tumors and CRISPRa-modulated mammary adipocytes dissected from patients resulted in lower cancer growth. Combined, our data suggests that AMT has enormous potential to treat a wide variety of cancers.

Adipocytes offer a unique ex vivo therapeutic system with many of the needed procedures already established in the clinic. Liposuction and fat transplantation are commonly used in many surgical procedures, such as aesthetic and reconstructive surgery. Due to successful engraftment, adipose tissue transplantation has progressively evolved, not only in plastic and reconstructive surgery, but also for therapeutic treatments (46). Several reports using rodent models have shown that BAT transplantation has beneficial metabolic outcomes (47-50). These also include the use of UCP1-CRISPRa modulation in human white preadipocytes to induce browning, followed by their transplantation in mice treated on a high-fat diet, leading to improved body weight, glucose tolerance and insulin sensitivity (34). Our work further showcases how these ‘brown’ adipocytes and adipose organoids can be utilized for cancer treatment.

There is a growing interest in carrying out human adipose tissue grafting by using adipose stem cells or progenitors due to their resistance to trauma and long-term survival following transplantation. One such example, is the use of CRISPRa to upregulate the relaxin family peptide receptor 1 (RXFP1) in adipose-derived stem cells and their transplantation in a diabetes mellitus induced erectile dysfunction rat model, showing amelioration of the erectile disfunction phenotype (51). Organoids could be advantageous for these cells as they provide a 3D-culture that can better recapitulate the heterogeneity of adipose tissue, respond better to endogenous stimuli and form tissue microenvironments that could more efficiently integrate with cancer cells following transplantation. Several groups have successfully grown adipose organoids from mouse adipose stem cells (33, 40, 52, 53). Here, we were able to culture human adipose organoids from preadipocytes using various conditions from previous studies (33, 40, 52, 53). These human adipose organoids exhibited mature adipocytes markers, including FABP4 and PLIN1. In addition, in our hands they could last up to a year after being subcutaneously implanted in mice, showing the potential for long-term treatment (data not shown). Finally, in our study we show that their CRISPRa modulation could significantly reduce tumor size, glycolysis and fatty acid metabolism and improve hypoxia and angiogenesis in cancer mouse models. Further development of these organoids and their modulation could likely improve these attributes and their therapeutic use for a wide range of diseases.

Our work builds on the recent observation that activating BAT, via cold exposure, increases adipocyte glucose uptake, lipid metabolism and significantly inhibits tumor progression (15). Here, instead of placing tumor models in cold conditions, we took advantage of CRISPRa to increase the expression of BAT key regulators, including UCP1, PPARGC1A, PRDM16 to engineer adipocytes to have increased glucose and fatty acid uptake and metabolism, and used them to deplete resources from cancer cells. Interestingly, and in line with the BAT cold activation results (15), implanting CRISPRa-modulated adipose organoids distal from the tumors also led to suppression of cancer growth, suggesting that complex surgical procedures for tumors with limited access might not be needed for this approach. This phenomenon might be due to additional mechanisms in which CRISPRa-modulated adipose organoids can suppress cancer growth. Previous studies have shown that hyperinsulinemia can lead to cancer growth due to insulin being a powerful mitogen and survival factor (54-56). For example, administration of Dapagliflozin, a SGTL2 inhibitor that lowers blood glucose, and a controlled-release mitochondrial protonophore (CRMP) suppress cancer growth in mice by reversing hyperinsulinemia (57). Since BAT is widely known to reduce whole-body blood glucose and insulin levels in humans (15, 49, 58), we hypothesize that the CRISPRa-modulated adipose organoids can reduce cancer progression by lowering plasma insulin levels. Indeed, our data, using both xenograft and genetic mouse models, shows that mice implanted with CRISPRa-modulated adipose organoids have reduced plasma insulin levels compared to dCas9-V64 control mice (FIG. 13G, 14F,H).

Amongst BAT-activating genes, UCP1 showed the most robust effect in terms of cancer suppression. It would be interesting to further develop this AMT approach to upregulate additional genes that could aid in cancer therapy. These could include for example upregulation of GLUT1 and GLUT4, that are the main glucose transporters in adipose cells, with GLUT4 being the most abundant and insulin responsive (59). Glucose metabolism associated genes, such as the transcription factor FOXO1 (60) and the G-coupled receptors, GPR20 and GPR120, which were implicated in improved glucose uptake and insulin resistance (61, 62), and AIFM2, which promotes glycolysis in BAT (63). Fatty acid oxidation associated genes, including for example the fatty acid transporter CD36, a key transporter for fatty acid oxidation, CPT1b, and the fatty acid breakdown enzyme, ACC1. Additional modifications could also be engineered in these adipocytes/adipose organoids, including for example utilization of their endocrine capabilities to secrete chemotherapeutic drugs or other cancer therapeutic associated compounds.

In this study, we used AAV-based CRISPRa to upregulate genes. However, it is worth noting that both upregulation and delivery could also be carried out using other modalities. For example, gene upregulation could be carried out using zinc fingers, TALENS, generation of specific mutations via regular CRISPR editing or base or prime editing in promoters or enhancers or standard overexpression using a cDNA mammalian expression construct of the gene of interest. Delivery could also be carried out with other viruses, such as lentivirus that is widely used for CAR-T therapy, but has a major caveat of genomic integration, or various non-viral nucleic acid delivery vehicles such as nanoparticles (64) or virus-like particles (VLPs) (65). Various drugs could also be used to upregulate specific genes in adipocytes in a global manner in cancer patients. In addition, downregulation of certain genes in adipocytes or adipose organoids, using CRISPRi, siRNA, CRISPR editing or other techniques could also be utilized for AMT. For example, a recent study used CRISPR to deplete the nuclear receptor interacting protein 1 (NRIP1) to make ‘brown’ adipocytes which upon implantation in mice decreased their adiposity on a high-fat diet (66).

A link between obesity, excess amount of WAT and cancer development and progression has been established, with nearly 40% of all cancer deaths in the United States being attributed to obesity (67). There are numerous proposed mechanisms in how WAT is linked to cancer development and progression, including chronic inflammation, hyperinsulinemia, steroid hormones, and adipokines (68-74). In addition, in glucose-rich conditions, cancer cells synthesize de novo FAs from intermediates of the glycolysis-TCA cycle (lipogenesis)(75, 76). The synthesized FAs are then utilized to synthesize triglycerides and are stored as lipid droplets in cancer cells. When energy is needed, these lipid droplets undergo lipolysis to release FAs which are subjected for b-oxidation (76-78). Since BAT is highly associated with improved glucose tolerance and insulin sensitivity (79), one could envision a personalized treatment development for obese cancer patients, whereby AMT is used not only to target cancer and its unique metabolism in these patients but also treat their metabolic disease. One major hurdle in our approach that needs to be taken into account is cancer-associated cachexia (41). While we did not observe weight loss in our mouse models, a longer treatment time could possibly lead to a reduction in body weight, as was shown for UCP1-CRISPRa mice on a high-fat diet (34). Modification of other genes than UCP1, removal of the implant after a certain time or having a molecular kill switch could all be potential solutions for cachexia if observed.

In summary, our results provide proof-of-principle results for a novel cancer therapeutic approach, termed AMT, that can be further improved and personalized for specific cancers and patients. Similar to chimeric antigen receptor (CAR)-T cell therapy, AMT can be readily used in the clinic as cells can be obtained from cancer patients via liposuction or other procedures, engineered and transplanted back into the same individual for therapeutic benefit. The utilization of adipocytes from dissected breast tissue, as performed in our study, further showcases the clinical utility of such an ex vivo approach. In particular, for breast cancer this could be particularly straightforward as many mastectomies are followed up by reconstructive surgery with autologous tissue (80), which could be manipulated prior to this procedure. Unlike T-cells, adipocytes have a lower immune response (34, 81) which could allow more straightforward development of ‘off-the-shelf’ adipocytes or adipose organoids for cancer and other treatments. Their ease of growth in culture, long lasting and robustness, and lower multiplicity (82) along with existing clinical procedures to remove and transplant them make them an exceptional cell type for cancer and other cellular-based disease therapies.

### Materials and Methods

### Human and Mouse Adipocytes

For adipocyte differentiation of human preadipocytes, human preadipocytes or mouse 3T3-L1 preadipocytes (both kind gifts from Dr. Hei Sook Sul, UC Berkeley) were cultured to 100% confluency in DMEM, supplemented with 10% FBS and fresh media were replaced. After 48 hours, cells were subjected to adipocyte differentiation by adding a cocktail of 3-isobutyl-1-methylxanthine (IBMX) (0.5M) (Sigma-Aldrich; 410957), dexamethasone (1 μM) (Sigma-Aldrich; D1756), and insulin (10 ug/ml) (Sigma-Aldrich; 19278). Media was replaced every two days with insulin containing DMEM complete media (Fisher Scientific; 11-965-118) during differentiation.

### Cancer Cell Lines

All the cancer cells were acquired from American Type Culture Collection (ATCC). MCF7 (ATCC; HTB-22) was cultured in Eagle's minimum essential medium (ATCC; 30-2003), supplemented with 10% FBS and 10 ug/ml human recombinant insulin (Sigma-Aldrich; 19278). MDB-MA-436 (ATCC; HTB-130) was cultured in Leibovitz's L-15 medium (ATCC; 30-2008) with 10 ug/ml insulin, 16 ug/ml glutathione (Sigma-Aldrich; G6013) and 10% FBS. SW-1417 (ATCC; CCL-238) was grown in Leibovitz's L-15 medium supplemented with 10% FBS. Panc 10.05 (ATCC; CRL-2547) was cultured in RPMI-1640 medium (ATCC; 30-2001) with 10 ug/ml human insulin and 15% FBS. DU-145 (ATCC; HTB-81) was cultured in Eagle's minimum essential medium with 10% FBS.

### CRISPRa AAV In Vitro Optimization

Five gRNAs targeting the promoter of human UCP1, PPARGC1A, and PRDM16 or mouse Ucp1 were designed using the Broad Institute CRISPick Tool (83) (Table S2). These guides were individually cloned into pAAV-U6-sasgRNA-CMV-mCherry-WPREpA (84) at the BstXI and Xhol restriction enzyme sites using the In-Fusion (Takara Bio; 638910) cloning methods as described in (84). 5×10⁵ human preadipocytes or mouse 3T3-L1 cells were plated into 12-well plate and were then subjected to adipocyte differentiation protocol after two days of confluency. At day four of differentiation, cells were transfected with 0.5 ug of dCas9-VP64 and 0.5 ug of gRNA plasmid using X-tremeGene (Sigma; 6366236001) and CombiMag reagent (OZ Biosciences; CM21000) following the manufacturer's protocol. At day eight of differentiation, cells were lysed with Trizol and RNA was collected and cDNA and qRT-PCR were performed, as mentioned in RNA Isolation and qRT-PCR section. The two gRNAs with the highest upregulation for each gene were packaged into rAAV-9 serotype virons. rAAV-9 serotype virons were produced by transfecting AAVpro 293T cell (Takara; 6322723) with pCMV-sadCas9-VP64 (Addgene; 115790) or pAAV-U6-sasgRNA-CMV-mCherry-WPREpA (84) along with packaging vectors, including PAAV2/9n (Addgene; 112865) and pHelper vectors using TransIT293 reagent (Mirus; 2700). After 72 hours, AAV particles were collected and purified using AAVpro Cell & Sup. Purification Kit Maxi (Takara; 6676) and quantified by the AAVpro Titration Kit (Takara; 6233). gRNA AAV viruses (1×10⁶ MOI) and dCas9-VP64 AAV (1×10⁶ MOI) were used to infect human and mouse differentiated adipocytes. After five days, RNA was collected, cDNA was made, and qRT-PCR was done as described below.

TABLE S2
Sequences of primers for RT-qPCR, cloning, genotyping and gRNAs.

| Gene/Use/sgRNA | Primers |
|---|---|
| GAPDH | GTC TCC TCT GAC TTC AAC AGC G (SEQ ID NO: 119) |
| | ACC ACC CTG TTG CTG TAG CCA A (SEQ ID NO: 120) |
| UCP1 | TGG AAT AGC GGC GTG CTT G (SEQ ID NO: 121) |
| | CTC ATC AGA TTG GGA GTA G (SEQ ID NO: 122) |
| PPARGC1A | CCA AAG GAT GCG CTC TCG TTC A (SEQ ID NO: 123) |
| | CGG TGT CTG TAG TGG CTT GAC T (SEQ ID NO: 124) |
| ADIPOQ | CAG GCC GTG ATG GCA GAG ATG (SEQ ID NO: 125) |
| | GGT TTC ACC GAT GTC TCC CTT AG (SEQ ID NO: 126) |
| PLIN1 | GCG GAA TTT GCT GCC AAC ACT C (SEQ ID NO: 127) |
| | AGA CTT CTG GGC TTG CTG GTG T (SEQ ID NO: 128) |
| FABP4 | ACG AGA GGA TGA TAA ACT GGT GG (SEQ ID NO: 129) |
| | GCG AAC TTC AGT CCA GGT CAA C (SEQ ID NO: 130) |
| MKI67 | GAA AGA GTG GCA ACC TGC CTT C (SEQ ID NO: 131) |
| | GCA CCA AGT TTT ACT ACA TCT GCC (SEQ ID NO: 132) |
| CD36 | CAG GTC AAC CTA TTG GTC AAG CC (SEQ ID NO: 133) |
| | GCC TTC TCA TCA CCA ATG GTC C (SEQ ID NO: 134) |
| GLUT4 | TGG GCT TCT TCA TCT TCA CC (SEQ ID NO: 135) |
| | GTG CTG GGT TTC ACC TCC T (SEQ ID NO: 136) |
| GCK | CAT CTC CGA CTT CCT GGA CAA G (SEQ ID NO: 137) |
| | TGG TCC AGT TGA GAA GGA TGC C (SEQ ID NO: 138) |
| CPT1b | TGT ATC GCC GTA AAC TGG ACC G (SEQ ID NO: 139) |
| | TGT CTG AGA GGT GCT GTA GCA C (SEQ ID NO: 140) |
| Gapdh | TGC ACC ACC AAC TGC TTA G (SEQ ID NO: 141) |
| | GGA TGC AGG GAT GAT GTT C (SEQ ID NO: 142) |
| Ucp1 | GTG AAC CCG ACA ACT TCC GAA (SEQ ID NO: 143) |
| | TGA AAC TCC GGC TGA GAA GAT (SEQ ID NO: 144) |
| Ppargc1a | ACA GCT TTC TGG GTG GAT TG (SEQ ID NO: 145) |
| | TGT CTC TGT GAG GAC CGC TA (SEQ ID NO: 146) |
| Prdm16 | TAT GGA GTG ACA TAG AGT GTG CT (SEQ ID NO: 147) |
| | CCA CTT CAA TCC ACC CAG AAA G (SEQ ID NO: 148) |
| Glut4 | GTA ACT TCA TTG TCG GCA TGG (SEQ ID NO: 149) |
| | AGC TGA GAT CTG GTC AAA CG (SEQ ID NO: 150) |
| Gck | GCA TCT CTG ACT TCC TGG ACA AG (SEQ ID NO: 151) |
| | CTT GGT CCA GTT GAG CAG GAT G (SEQ ID NO: 152) |
| Cd36 | GGA CAT TGA GAT TCT TTT CCT CTG (SEQ ID NO: 153) |
| | GCA AAG GCA TTG GCT GGA AGA AC (SEQ ID NO: 154) |
| Cpt1b | ATG TAT CGC CGC AAA CTG GAC C (SEQ ID NO: 155) |
| | CTC TGA GAG GTG CTG TAG CAA G (SEQ ID NO: 156) |
| Mki67 | GAG GAG AAA CGC CAA CCA AGA G (SEQ ID NO: 157) |
| | TTT GTC CTC GGT GGC GTT ATC C (SEQ ID NO: 158) |
| MTOR | AGC ATC GGA TGC TTA GGA GTG G (SEQ ID NO: 159) |
| | CAG CCA GTC ATC TTT GGA GAC C (SEQ ID NO: 160) |
| CK5 | ACG TCC ACT CTG CGA GAA TG (SEQ ID NO: 161) |
| | CGA TCA GGC TGT ACG TCA TCT (SEQ ID NO: 162) |
| CK8 | ACA AGG TAG AGC TGG AGT CTC G (SEQ ID NO: 163) |
| | AGC ACC ACA GAT GTG TCC GAG A (SEQ ID NO: 164) |
| CK17 | ATC CTG CTG GAT GTG AAG ACG C (SEQ ID NO: 165) |
| | TCC ACA ATG GTA CGC ACC TGA C (SEQ ID NO: 166) |
| UPP1 | CTC CAA CGT CAC TAT CAT CCG C (SEQ ID NO: 167) |
| | CTG CCT TGA AGC AGG TAT CCA C (SEQ ID NO: 168) |
| Cas9 | ATC ACCC CCC ACC AGA TCA AGC (SEQ ID NO: 169) |
| | GTC CTT GTC GTA CAG GCC GTT CA (SEQ ID NO: 170) |
| UCP1 gRNA-1 | CCCGAGGCACCGAGCGAGAAT (SEQ ID NO: 1) |
| UCP1 gRNA-2 | GCAGGGCTCCCGAGGCACCGA (SEQ ID NO: 2) |
| UCP1 gRNA-3 | AGGGCTCCCGAGGCACCGAGC (SEQ ID NO: 3) |
| UCP1 gRNA-4 | ACCGAGCGAGAATGGGAATGG (SEQ ID NO: 4) |
| UCP1 gRNA-5 | GCACCGAGCGAGAATGGGAAT (SEQ ID NO: 5) |
| PPARGC1a gRNA-1 | CACTGAAGCAGAGGGCTGCCT (SEQ ID NO: 6) |
| PPARGC1a gRNA-2 | TTAGAGCAGCAAGCTGCACAG (SEQ ID NO: 7) |
| PPARGC1a gRNA-3 | GTTAGAGCAGCAAGCTGCACA (SEQ ID NO: 8) |
| PPARGC1a gRNA-4 | GGGCTGCCTTTGAGTGACGTC (SEQ ID NO: 9) |
| PPARGC1a gRNA-5 | AGTTAGAGCAGCAAGCTGCAC (SEQ ID NO: 10) |
| PRDM16 gRNA-1 | CGGCGGCGGCGGCGCGACGAT (SEQ ID NO: 11) |
| PRDM16 gRNA-2 | GGCGGCGCGACGATGAGGATG (SEQ ID NO: 12) |
| PRDM16 gRNA-3 | GCCGCCGCCGCCGCCTCGGCG (SEQ ID NO: 13) |
| PRDM16 gRNA-4 | CGCCGCCGCCGCCGCCTCGGC (SEQ ID NO: 14) |
| PRDM16 gRNA-5 | GGCGGCGGCGGCGGCGCGACG (SEQ ID NO: 15) |
| Ucp1 gRNA-1 | GGGACTTGGCAGGGGCGTGCC (SEQ ID NO: 171) |
| Ucp1 gRNA-2 | CTGAGCCGGCCCAGGTCTCCA (SEQ ID NO: 172) |
| Ucp1 gRNA-3 | CAGGTCTCCAAAGAGCTGCTA (SEQ ID NO: 173) |
| Ucp1 gRNA-4 | CTTTGGGAGTGACGCGCGGCT (SEQ ID NO: 174) |
| Ucp1 gRNA-5 | GGCTTTGGGAGTGACGCGCGG (SEQ ID NO: 175) |
| UPP1 gRNA-1 | GCGAACTCAGCTGACCGCATC (SEQ ID NO: 176) |
| UPP1 gRNA-2 | GGGCTTGTCTGCGGGATGGCC (SEQ ID NO: 177) |
| UPP1 gRNA-3 | GATGGCCCAGGATGCGGTCAG (SEQ ID NO: 178) |
| UPP1 gRNA-4 | GGTCAGCTGAGTTCGCCGGCC (SEQ ID NO: 179) |
| UPP1 gRNA-5 | AGTTCGCCGGCCCAGGGCAGG (SEQ ID NO: 180) |

RNA Isolation and qRT-PCR

Total RNA from sorted or cultured cells was extracted using Trizol reagent (ThermoFisher; 15596026). Reverse transcription was performed with 1 μg of total RNA using qScript cDNA Synthesis Kit (Quantabio; 95047) following the manufacturer's protocol. qRT-PCR was performed on QuantStudio 6 Real Time PCR system (ThermoFisher) using Sso Fast (Bio-Rad; 1705205). Statistical analysis was performed using ddct method with GAPDH or Gapdh primers as control (see primer sequences in Table S2). Gene expression results were generated using mean values for over n=4-6 biological replicates.

### Seahorse Assay

Human preadipocytes (5×10⁵ cell/well) were plated in 12-well tissue culture plates and subjected to adipocyte differentiation protocol. Adipocytes and cancer cell lines were trypsinized and reseeded in XF96 plates (Agilent; 102905-100) at 20K cells per well and assayed the next day. On the day of experiments, the cells were washed two times and maintained in XF base medium (Agilent; 1033334) supplemented either with 1 mM sodium pyruvate (ThermoFisher; 11360070) and 17.5 mM glucose (Sigma-Aldrich; G7021), for the mitochondria stress test or with 2 mM glutamine (StemCell technologies; 07100) for the glycolysis test. For FAO tests, cells were washed and incubated with substrate-limiting medium DMEM (Corning; 17-207-CV) supplemented with 0.5 mM glucose, 1 mM glutaMAX (ThermoFisher; 3050061), 0.5 mM carnitine (Sigma-Aldrich; C0283), and 1% FBS. Cells were then incubated at 37° C. in a non-CO2 incubator for 1 hour. The assay was performed using Seahorse XFe96 analyzer (Agilent; XFe96). Oxygen consumption measured under 1.5 M oligomycin (Sigma-Aldrich; 75351), 2 μM FCCP (Sigma-Aldrich; C2920), and 0.5 μM of Rotenone (Sigma-Aldrich; R8875)/Antimycin (Sigma-Aldrich; A8674). Extracellular acidification rate (ECAR) was measured under 10 mM glucose, 1 uM oligomycin, and 50 mM 2-deoxyglucose (Sigma-Aldrich; D8375).

### Glucose Uptake Assay

Human preadipocytes (3×10⁵ cell/well) were plated in 96-well tissue culture plates and subjected to adipocyte differentiation protocol. One day before the assay, media was replaced with serum-free media. On the day of the assay, using glucose uptake-Glo assay (Promega; J1342), cells were incubated with 1 nM insulin (Sigma-Aldrich; 19278) for 1 hour prior to the assay following the manufacturer's protocol.

Co-Culturing CRISPRa-Modulated Adipocytes with Cancer Cells

5×10⁵ human preadipocytes were plated in 12-well tissue culture plates and subjected to adipocyte differentiation protocol. At day 2 of differentiation, cells were transduced with gRNA AAV viruses (1×10⁶ MOI) and dCas9-VP64 AAV (1×10⁶ MOI). After 6 days, cells were collected and replated into the upper-well of 12-well transwell plate (Fisher Scientific; 07-200-150) in which 3×10⁵ cancer cells were plated in the lower-well a day earlier. The cells were culture in adipocyte differentiation media, described previously, and designated media for each cancer cell line (1:1 ratio). After three days, cancer cells were collected for imaging and seahorse assay. RNA was collected, and cDNA was made, and qRT-PCR was carried out as previously described. Differential expression was determined using the ddct method with GAPDH primers as control (primer sequences in Table S2).

### Human and Mouse Adipose Organoid Culture

Human or mouse 3T3-L1 preadipocytes (0.5×10⁶ cells) were plated in 96-well Nunclon Sphera ULA U-bottom plates in (ThermoFisher; 174929). Organoids formed after 48 hours and were then differentiated to adipose organoids using a differentiation cocktail containing 3-isobutyl-1-methylxanthine (IBMX) (0.5M), dexamethasone (1 uM), and insulin (10 ug/ml). Adipocytes formed 21-days post differentiation. Human adipose organoids were then transduced with gRNA AAV viruses (1×10⁶ MOI) and dCas9-VP64 AAV (1×10⁶ MOI). After 5 days, organoids were collected and mixed with Matrigel (Corning; 354234) and subcutaneously injected into mice (10 organoids/mouse).

### Co-Transplantation of CRISPRa-Modulated Human Adipose Organoids and Cancer Cells

All animal studies were carried out in accordance with the University of California San Francisco Institutional Animal Care and Use Committee protocol number AN197608. Mice were housed in a 12:12 light-dark cycle, and chow diet (Envigo; 2018S) and water were provided ad libitum. Five-week-old immune-deficient SCID mice (JAX; 001303) were anesthetized using isoflurane and subcutaneously injected with 2×10⁶-6×10⁶ cancer cells in phosphate buffered saline (PSB). After 6-12 weeks, depending on the cancer cell lines, we subcutaneously injected CRISPRa AAV human adipocytes or adipose organoids to a site adjacent to the tumor. Following 4-6 weeks, mice were euthanized, and tumors and adipose implants were collected. We measured tumor size with calipers and tumor volume according to standard formula (length×width2×0.52).

### Immunofluorescence

A portion of isolated tumors were fixed with 1% paraformaldehyde for two hours and prepared for cryostat sectioning. Each sample were cut into 5 umm thick sections and blocked with 3% BSA (Miltenyi Biotech; 130-091-376) blocking solution for 30 minutes. For Ki-67 staining, slides were permeabilized with 1% triton X-100 PBS solution for 15 minutes and washed with 0.1% Tween20 PBS solution prior to blocking. Slides were then incubated with primary antibodies (information and concentration listed in Table S3) overnight at 40C and washed 3× with 0.1% Tween20 PBS. The slides were then incubated with secondary antibodies (information and concentration listed in Table S3) for 60 minutes at room temperature and then washed 3× with 0.1% Tween20 PBS. Images were obtained using a confocal microscope (Zeiss; LSM880) and analyzed with Fiji (85).

TABLE S3
List of antibodies.

| Name | Vendor | Concentration | Catalog number |
|---|---|---|---|
| Ki67 (SolA15) | Fisher Scientific | 5 ug/ml | 14-5698-82 |
| Carbonic Anhydrase | Fisher Scientific | 3 ug/ml | AF2188 |
| CD31 | Fisher Scientific | 10 ug/ml | BBA7 |
| Caspase 3 | Cell Signaling | 1:400 | 9661 |
| CK19 | Abcam | 1:200 | Ab203444 |
| UCP1 | R&D | 0.5 ug/ml | MAB6158 |
| Goat anti-rat, Alexa | Fisher Scientific | 1:1000 | A21247 |
| Flour 647 | | | |
| Goat anti-mouse, Alexa | Life | 1:500 | A11032 |
| Fluor 594 | Technologies | | |
| Donkey anti-goat, Alexa | Fisher Scientific | 1:1000 | A11055 |
| Fluor 594 | | | |

### CRISPRa-Modulated Mouse Adipose Organoid Implantation in Pancreatic and Breast Cancer Genetic Mouse Models

KPC, tamoxifen-inducible” is a KrasLSL-G12D;p53LoxP;Pdx1-CreER triple mutant model of tamoxifen-inducible pancreatic ductal adenocarcinoma (PDAC) on an C57/BL6 background was acquired at the Jackson Laboratory (032429). To induce Cre recombination, tamoxifen was administered to pups via lactation following oral gavage of the mother with 6 mg tamoxifen (suspended in corn oil) at postnatal days 0, 1, 2 and 4. We orthotopically implanted mouse adipose organoids (10 organoids/mouse) mixed with Matrigel (Corning; 354234) near the pancreas in 4-week-old mice, using a similar protocol describe in (86) and dissected the pancreas 6 weeks after the implantation. The MMTV-PyMT breast cancer mice were acquired from the Jackson Laboratory (002374). Four-week-old female mice were implanted with mouse adipose organoids (10 organoids/mouse) mixed with Matrigel (Corning; 354234) and subcutaneously injected into mice either at the third nipple or the back. The tumors were collected six weeks post implantation.

### Cancer Organoids

Organoids were generated from breast tumor tissue or tumors from malignant effusions. Patients were consented for specimen collection using IRB-approved tissue collection protocols at the University of California, San Francisco and the Brigham & Women's Hospital. Surgical specimens were obtained from the UCSF Medical Center or Brigham & Women's Hospital on the day of their procedure, viably frozen as tissue pieces, or used to generate formalin-fixed, paraffin-embedded (FFPE) sections or organoid cultures. Each tissue was minced using razor blades and digested in a solution containing DMEM/F12 (Gibco; 11330), 2 mM GlutaMax (Gibco; 35050), 10 mM HEPES (Gibco; 15630), 50 U/mL Penicillin-Streptomycin (Gibco; 15070), and 1 mg/ml collagenase XI (Sigma-Aldrich; C9407). Tissue digestion was performed at 37° C. with constant shaking at 150 rpm for 1-2 hours. Cells were then pelleted by centrifugation, further dissociated by sequentially pipetting with 10, 5, and 1-ml pipette tips, and recentrifuged. The resulting cell pellet was used directly to establish organoid cultures by embedding in basement membrane extract, allowing this to harden at 37° C. for 20 minutes to form a hydrogel dome, and then overlaying this dome with Type 1 Organoid Medium as previously describe (87, 88). All metastatic cancer organoids were derived from malignant pleural effusions that were collected from metastatic breast cancer patients undergoing thoracentesis at the UCSF Medical Center. The fluid samples were placed on ice within four hours after collection. Organoids were generated by washing malignant effusions with PBS, collecting tumor spheroids by centrifugation, and incubating with 3-5 ml RBC lysis buffer (BioLegend; 420301) for 10-15 minutes when there were visible RBCs, followed by embedding the cell pellet in organoid culture as described above.

### Mammary Gland Adipocyte Isolation

For adipocyte extraction, mammary gland adipose tissues from excess tissue removed during breast surgeries were washed with PBS three times and mechanically minced into smaller pieces. The tissue mixture was incubated with PBS with 3% BSA and Collagenase I (1 mg/ml) for 45 minutes at 37° C., centrifuged and the top lipid layer was discarded. The remaining mixture was filtered twice through a 200 μm strainer and centrifuged. Mature adipocytes were isolated and washed twice with PBS and were plated in suspension in 6 well plates in high glucose DMEM media supplemented with 10% FBS. For transduction of CRISPRa, UCP1 AAV9 viruses (1×10⁶ MOI) and dCas9-VP64 AAV9 (1×10⁶ MOI) were mixed with 20 ul of AdeoMag (OzBiosciences, AM71000) and adipocytes and incubated for 15 minutes. The virus and cell mixture were distributed onto 6-well plate placed upon the magnetic plate and incubated for 15 more minutes.

### Co-Culturing of Cancer Organoids and Adipocytes

Five days post infection of CRISPRa AAV9, adipocytes were placed in a tissue transwell culture plate (Fisher Scientific; 07-200-150) of tumor organoids in 1:1 ratio of adipocyte media and breast cancer organoid media. Cells were incubated for up to seven days and then examined for adipocyte and tumor phenotypes.

### REFERENCES

1. X. Lin, Z. Xiao, T. Chen, S. H. Liang, and H. Guo, Glucose Metabolism on Tumor Plasticity, Diagnosis, and Treatment. Front Oncol. 10, p. 317 (2020).
2. N. Hay, Reprogramming glucose metabolism in cancer: can it be exploited for cancer therapy? Nat Rev Cancer. 16(10), p. 635-49 (2016).
3. M. V. Liberti and J. W. Locasale, The Warburg Effect: How Does it Benefit Cancer Cells? Trends Biochem Sci. 41(3), p. 211-218 (2016).
4. S. R. Nagarajan, L. M. Butler, and A. J. Hoy, The diversity and breadth of cancer cell fatty acid metabolism. Cancer Metab. 9(1), p. 2 (2021).
5. W. Palm and C. B. Thompson, Nutrient acquisition strategies of mammalian cells. Nature. 546(7657), p. 234-242 (2017).
6. D. DeWaal, V. Nogueira, A. R. Terry, K. C. Patra, S. M. Jeon, G. Guzman, J. Au, C. P. Long, M. R. Antoniewicz, and N. Hay, Hexokinase-2 depletion inhibits glycolysis and induces oxidative phosphorylation in hepatocellular carcinoma and sensitizes to metformin. Nat Commun. 9(1), p. 446 (2018).
7. D. A. Chan, P. D. Sutphin, P. Nguyen, S. Turcotte, E. W. Lai, A. Banh, G. E. Reynolds, J. T. Chi, J. Wu, D. E. Solow-Cordero, M. Bonnet, J. U. Flanagan, D. M. Bouley, E. E. Graves, W. A. Denny, M. P. Hay, and A. J. Giaccia, Targeting GLUT1 and the Warburg effect in renal cell carcinoma by chemical synthetic lethality. Sci Transl Med. 3(94), p. 94ra70 (2011).
8. Y. Liu, Y. Cao, W. Zhang, S. Bergmeier, Y. Qian, H. Akbar, R. Colvin, J. Ding, L. Tong, S. Wu, J. Hines, and X. Chen, A small-molecule inhibitor of glucose transporter 1 downregulates glycolysis, induces cell-cycle arrest, and inhibits cancer cell growth in vitro and in vivo. Mol Cancer Ther. 11(8), p. 1672-82 (2012).
9. L. P. Fernandez, M. Gomez de Cedron, and A. Ramirez de Molina, Alterations of Lipid Metabolism in Cancer: Implications in Prognosis and Treatment. Front Oncol. 10, p. 577420 (2020).
10. W. Khan, D. Augustine, R. S. Rao, S. Patil, K. H. Awan, S. V. Sowmya, V. C. Haragannavar, and K. Prasad, Lipid metabolism in cancer: A systematic review. J Carcinog. 20, p. 4 (2021).
11. W. Wang, L. Bai, W. Li, and J. Cui, The Lipid Metabolic Landscape of Cancers and New Therapeutic Perspectives. Front Oncol. 10, p. 605154 (2020).
12. M. T. Snaebjornsson, S. Janaki-Raman, and A. Schulze, Greasing the Wheels of the Cancer Machine: The Role of Lipid Metabolism in Cancer. Cell Metab. 31(1), p. 62-76 (2020).
13. P. Mason, B. Liang, L. Li, T. Fremgen, E. Murphy, A. Quinn, S. L. Madden, H. P. Biemann, B. Wang, A. Cohen, S. Komarnitsky, K. Jancsics, B. Hirth, C. G. Cooper, E. Lee, S. Wilson, R. Krumbholz, S. Schmid, Y. Xiang, M. Booker, J. Lillie, and K. Carter, SCD1 inhibition causes cancer cell death by depleting mono-unsaturated fatty acids. PLOS One. 7(3), p. e33823 (2012).
14. N. V. Guseva, O. W. Rokhlin, R. A. Glover, and M. B. Cohen, TOFA (5-tetradecyl-oxy-2-furoic acid) reduces fatty acid synthesis, inhibits expression of AR, neuropilin-1 and Mcl-1 and kills prostate cancer cells independent of p53 status. Cancer Biol Ther. 12(1), p. 80-5 (2011).
15. T. Seki, Y. Yang, X. Sun, S. Lim, S. Xie, Z. Guo, W. Xiong, M. Kuroda, H. Sakaue, K. Hosaka, X. Jing, M. Yoshihara, L. Qu, X. Li, Y. Chen, and Y. Cao, Brown-fat-mediated tumour suppression by cold-altered global metabolism. Nature. 608(7922), p. 421-428 (2022).
16. M. E. Symonds, P. Aldiss, M. Pope, and H. Budge, Recent advances in our understanding of brown and beige adipose tissue: the good fat that keeps you healthy. F1000Res. 7 (2018).
17. K. A. Virtanen, M. E. Lidell, J. Orava, M. Heglind, R. Westergren, T. Niemi, M. Taittonen, J. Laine, N.J. Savisto, S. Enerback, and P. Nuutila, Functional brown adipose tissue in healthy adults. N Engl J Med. 360(15), p. 1518-25 (2009).
18. B. Cannon and J. Nedergaard, Brown adipose tissue: function and physiological significance. Physiol Rev. 84(1), p. 277-359 (2004).
19. M. Klingenberg, Uncoupling protein—a useful energy dissipator. J Bioenerg Biomembr. 31(5), p. 419-30 (1999).
20. D. Suzuki, Y. Murata, and S. Oda, Changes in Ucp1, D2 (Dio2) and Glut4 (Slc2a4) mRNA expression in response to short-term cold exposure in the house musk shrew (Suncus murinus). Exp Anim. 56(4), p. 279-88 (2007).
21. K. S. Vimaleswaran, V. Radha, R. Deepa, and V. Mohan, Absence of Association of Metabolic Syndrome with PPARGC1A, PPARG and UCP1 Gene Polymorphisms in Asian Indians. Metab Syndr Relat Disord. 5(2), p. 153-62 (2007).
22. H. M. Feldmann, V. Golozoubova, B. Cannon, and J. Nedergaard, UCP1 ablation induces obesity and abolishes diet-induced thermogenesis in mice exempt from thermal stress by living at thermoneutrality. Cell Metab. 9(2), p. 203-9 (2009).
23. C. Tabuchi and H. S. Sul, Corrigendum: Signaling Pathways Regulating Thermogenesis. Front Endocrinol (Lausanne). 12, p. 698619 (2021).
24. D. Yi, J. M. Dempersmier, H. P. Nguyen, J. A. Viscarra, J. Dinh, C. Tabuchi, Y. Wang, and H. S. Sul, Zc3h10 Acts as a Transcription Factor and Is Phosphorylated to Activate the Thermogenic Program. Cell Rep. 29(9), p. 2621-2633 e4 (2019).
25. P. Puigserver and B. M. Spiegelman, Peroxisome proliferator-activated receptor-gamma coactivator 1 alpha (PGC-1 alpha): transcriptional coactivator and metabolic regulator. Endocr Rev. 24(1), p. 78-90 (2003).
26. J. Lin, P. H. Wu, P. T. Tarr, K. S. Lindenberg, J. St-Pierre, C. Y. Zhang, V. K. Mootha, S. Jager, C. R. Vianna, R. M. Reznick, L. Cui, M. Manieri, M. X. Donovan, Z. Wu, M. P. Cooper, M. C. Fan, L. M. Rohas, A. M. Zavacki, S. Cinti, G. I. Shulman, B. B. Lowell, D. Krainc, and B. M. Spiegelman, Defects in adaptive energy metabolism with CNS-linked hyperactivity in PGC-1 alpha null mice. Cell. 119(1), p. 121-35 (2004).
27. S. Kajimura, Promoting brown and beige adipocyte biogenesis through the PRDM16 pathway. Int J Obes Suppl. 5(Suppl 1), p. S11-4 (2015).
28. M. J. Harms, H. W. Lim, Y. Ho, S. N. Shapira, J. Ishibashi, S. Rajakumari, D. J. Steger, M. A. Lazar, K. J. Won, and P. Seale, PRDM16 binds MED1 and controls chromatin architecture to determine a brown fat transcriptional program. Genes Dev. 29(3), p. 298-307 (2015).
29. H. Ohno, K. Shinoda, B. M. Spiegelman, and S. Kajimura, PPARgamma agonists induce a white-to-brown fat conversion through stabilization of PRDM16 protein. Cell Metab. 15(3), p. 395-404 (2012).
30. S. Kajimura, P. Seale, K. Kubota, E. Lunsford, J. V. Frangioni, S. P. Gygi, and B. M. Spiegelman, Initiation of myoblast to brown fat switch by a PRDM16-C/EBP-beta transcriptional complex. Nature. 460(7259), p. 1154-8 (2009).
31. P. Seale, B. Bjork, W. Yang, S. Kajimura, S. Chin, S. Kuang, A. Scime, S. Devarakonda, H. M. Conroe, H. Erdjument-Bromage, P. Tempst, M. A. Rudnicki, D. R. Beier, and B. M. Spiegelman, PRDM16 controls a brown fat/skeletal muscle switch. Nature. 454(7207), p. 961-7 (2008).
32. S. Kajimura, P. Seale, T. Tomaru, H. Erdjument-Bromage, M. P. Cooper, J. L. Ruas, S. Chin, P. Tempst, M. A. Lazar, and B. M. Spiegelman, Regulation of the brown and white fat gene programs through a PRDM16/CtBP transcriptional complex. Genes Dev. 22(10), p. 1397-409 (2008).
33. P. Seale, S. Kajimura, W. Yang, S. Chin, L. M. Rohas, M. Uldry, G. Tavernier, D. Langin, and B. M. Spiegelman, Transcriptional control of brown fat determination by PRDM16. Cell Metab. 6(1), p. 38-54 (2007).
34. C. H. Wang, M. Lundh, A. Fu, R. Kriszt, T. L. Huang, M. D. Lynes, L. O. Leiria, F. Shamsi, J. Darcy, B. P. Greenwood, N. R. Narain, V. Tolstikov, K. L. Smith, B. Emanuelli, Y. T. Chang, S. Hagen, N,N. Danial, M. A. Kiebish, and Y. H. Tseng, CRISPR-engineered human brown-like adipocytes prevent diet-induced obesity and ameliorate metabolic syndrome in mice. Sci Transl Med. 12(558)(2020).
35. H. K. Kim, S. Min, M. Song, S. Jung, J. W. Choi, Y. Kim, S. Lee, S. Yoon, and H. H. Kim, Deep learning improves prediction of CRISPR-Cpf1 guide RNA activity. Nat Biotechnol. 36(3), p. 239-241 (2018).
36. J. Flint and T. Shenk, Viral transactivating proteins. Annu Rev Genet. 31, p. 177-212 (1997).
37. Z. Wu, H. Yang, and P. Colosi, Effect of genome size on AAV vector packaging. Mol Ther. 18(1), p. 80-6 (2010).
38. R. Bates, W. Huang, and L. Cao, Adipose Tissue: An Emerging Target for Adeno-associated Viral Vectors. Mol Ther Methods Clin Dev. 19, p. 236-249 (2020).
39. H. A. Strobel, T. Gerton, and J. B. Hoying, Vascularized adipocyte organoid model using isolated human microvessel fragments. Biofabrication. 13(3)(2021).
40. J. Taylor, J. Sellin, L. Kuerschner, L. Krahl, Y. Majlesain, I. Forster, C. Thiele, H. Weighardt, and E. Weber, Generation of immune cell containing adipose organoids for in vitro analysis of immune metabolism. Sci Rep. 10(1), p. 21104 (2020).
41. S. Kir and B. M. Spiegelman, Cachexia & Brown Fat: A Burning Issue in Cancer. Trends Cancer. 2(9), p. 461-463 (2016).
42. R. Maddipati and B. Z. Stanger, Pancreatic Cancer Metastases Harbor Evidence of Polyclonality. Cancer Discov. 5(10), p. 1086-97 (2015).
43. C. T. Guy, R. D. Cardiff, and W. J. Muller, Induction of mammary tumors by expression of polyomavirus middle T oncogene: a transgenic mouse model for metastatic disease. Mol Cell Biol. 12(3), p. 954-61 (1992).
44. J. F. Dekkers, E. J. van Vliet, N. Sachs, J. M. Rosenbluth, O. Kopper, H. G. Rebel, E. J. Wehrens, C. Piani, J. E. Visvader, C. S. Verissimo, S. F. Boj, J. S. Brugge, H. Clevers, and A. C. Rios, Long-term culture, genetic manipulation and xenotransplantation of human normal and breast cancer organoids. Nat Protoc. 16(4), p. 1936-1965 (2021).
45. M. Picon-Ruiz, J. A. Marchal, and J. M. Slingerland, Obtaining Human Breast Adipose Cells for Breast Cancer Cell Co-culture Studies. STAR Protoc. 1(3), p. 100197 (2020).
46. L. Shukla, Y. Yuan, R. Shayan, D. W. Greening, and T. Karnezis, Fat Therapeutics: The Clinical Capacity of Adipose-Derived Stem Cells and Exosomes for Human Disease and Tissue Regeneration. Front Pharmacol. 11, p. 158 (2020).
47. J. D. White, R. S. Dewal, and K. I. Stanford, The beneficial effects of brown adipose tissue transplantation. Mol Aspects Med. 68, p. 74-81 (2019).
48. X. Liu, Z. Zheng, X. Zhu, M. Meng, L. Li, Y. Shen, Q. Chi, D. Wang, Z. Zhang, C. Li, Y. Li, Y. Xue, J. R. Speakman, and W. Jin, Brown adipose tissue transplantation improves whole-body energy metabolism. Cell Res. 23(6), p. 851-4 (2013).
49. K. I. Stanford, R. J. Middelbeek, K. L. Townsend, D. An, E. B. Nygaard, K. M. Hitchcox, K. R. Markan, K. Nakano, M. F. Hirshman, Y. H. Tseng, and L. J. Goodyear, Brown adipose tissue regulates glucose homeostasis and insulin sensitivity. J Clin Invest. 123(1), p. 215-23 (2013).
50. X. Liu, S. Wang, Y. You, M. Meng, Z. Zheng, M. Dong, J. Lin, Q. Zhao, C. Zhang, X. Yuan, T. Hu, L. Liu, Y. Huang, L. Zhang, D. Wang, J. Zhan, H. Jong Lee, J. R. Speakman, and W. Jin, Brown Adipose Tissue Transplantation Reverses Obesity in Ob/Ob Mice. Endocrinology. 156(7), p. 2461-9 (2015).
51. T. Sun, W. Xu, B. Tu, T. Wang, J. Liu, K. Liu, and Y. Luan, Engineered Adipose-Derived Stem Cells Overexpressing RXFP1 via CRISPR Activation Ameliorate Erectile Dysfunction in Diabetic Rats. Antioxidants. 12(1), p. 171 (2023).
52. M. C. Rudolph, E. A. Wellberg, and S. M. Anderson, Adipose-depleted mammary epithelial cells and organoids. J Mammary Gland Biol Neoplasia. 14(4), p. 381-6 (2009).
53. H. A. Strobel, T. Gerton, and J. B. Hoying, Vascularized adipocyte organoid model using isolated human microvessel fragments. Biofabrication. (2021).
54. C. J. Currie, C. D. Poole, and E. A. Gale, The influence of glucose-lowering therapies on cancer risk in type 2 diabetes. Diabetologia. 52(9), p. 1766-77 (2009).
55. L. G. Hemkens, U. Grouven, R. Bender, C. Gunster, S. Gutschmidt, G. W. Selke, and P. T. Sawicki, Risk of malignancies in patients with diabetes treated with human insulin or insulin analogues: a cohort study. Diabetologia. 52(9), p. 1732-44 (2009).
56. I. F. Godsland, Insulin resistance and hyperinsulinaemia in the development and progression of cancer. Clin Sci (Lond). 118(5), p. 315-32 (2009).
57. A. R. Nasiri, M. R. Rodrigues, Z. Li, B. P. Leitner, and R. J. Perry, SGLT2 inhibition slows tumor growth in mice by reversing hyperinsulinemia. Cancer Metab. 7, p. 10 (2019).
58. M. Chondronikola, E. Volpi, E. Borsheim, C. Porter, P. Annamalai, S. Enerback, M. E. Lidell, M. K. Saraf, S. M. Labbe, N. M. Hurren, C. Yfanti, T. Chao, C. R. Andersen, F. Cesani, H. Hawkins, and L. S. Sidossis, Brown adipose tissue improves whole-body glucose homeostasis and insulin sensitivity in humans. Diabetes. 63(12), p. 4089-99 (2014).
59. A. Chadt and H. Al-Hasani, Glucose transporters in adipose tissue, liver, and skeletal muscle in metabolic health and disease. Pflugers Arch. 472(9), p. 1273-1298 (2020).
60. S. Kousteni, FoxO1, the transcriptional chief of staff of energy metabolism. Bone. 50(2), p. 437-43 (2012).
61. M. Schilperoort, A. D. van Dam, G. Hoeke, I. G. Shabalina, A. Okolo, A. C. Hanyaloglu, L. H. Dib, I. M. Mol, N. Caengprasath, Y. W. Chan, S. Damak, A. R. Miller, T. Coskun, B. Shimpukade, T. Ulven, S. Kooijman, P. C. Rensen, and M. Christian, The GPR120 agonist TUG-891 promotes metabolic health by stimulating mitochondrial respiration in brown fat. EMBO Mol Med. 10(3)(2018).
62. S. Satapati, Y. Qian, M. S. Wu, A. Petrov, G. Dai, S. P. Wang, Y. Zhu, X. Shen, E. S. Muise, Y. Chen, E. Zycband, A. Weinglass, J. Di Salvo, J. S. Debenham, J. M. Cox, P. Lan, V. Shah, S. F. Previs, M. Erion, D. E. Kelley, L. Wang, A. D. Howard, and J. Shang, GPR120 suppresses adipose tissue lipolysis and synergizes with GPR40 in antidiabetic efficacy. J Lipid Res. 58(8), p. 1561-1578 (2017).
63. H. P. Nguyen, D. Yi, F. Lin, J. A. Viscarra, C. Tabuchi, K. Ngo, G. Shin, A. Y. Lee, Y. Wang, and H. S. Sul, Aifm2, a NADH Oxidase, Supports Robust Glycolysis and Is Required for Cold- and Diet-Induced Thermogenesis. Mol Cell. 77(3), p. 600-617 e4 (2020).
64. J. Chen, Z. Guo, H. Tian, and X. Chen, Production and clinical development of nanoparticles for gene delivery. Mol Ther Methods Clin Dev. 3, p. 16023 (2016).
65. S. Banskota, A. Raguram, S. Suh, S. W. Du, J. R. Davis, E. H. Choi, X. Wang, S. C. Nielsen, G. A. Newby, P. B. Randolph, M. J. Osborn, K. Musunuru, K. Palczewski, and D. R. Liu, Engineered virus-like particles for efficient in vivo delivery of therapeutic proteins. Cell. 185(2), p. 250-265 e16 (2022).
66. E. Tsagkaraki, S. M. Nicoloro, T. DeSouza, J. Solivan-Rivera, A. Desai, L. M. Lifshitz, Y. Shen, M. Kelly, A. Guilherme, F. Henriques, N. Amrani, R. Ibraheim, T. C. Rodriguez, K. Luk, S. Maitland, R. H. Friedline, L. Tauer, X. Hu, J. K. Kim, S. A. Wolfe, E. J. Sontheimer, S. Corvera, and M. P. Czech, CRISPR-enhanced human adipocyte browning as cell therapy for metabolic disease. Nat Commun. 12(1), p. 6931 (2021).
67. C. B. Steele, C. C. Thomas, S. J. Henley, G. M. Massetti, D. A. Galuska, T. Agurs-Collins, M. Puckett, and L. C. Richardson, Vital Signs: Trends in Incidence of Cancers Associated with Overweight and Obesity-United States, 2005-2014. MMWR Morb Mortal Wkly Rep. 66(39), p. 1052-1058 (2017).
68. A. G. Renehan, M. Zwahlen, and M. Egger, Adiposity and cancer risk: new mechanistic insights from epidemiology. Nat Rev Cancer. 15(8), p. 484-98 (2015).
69. G. Paz-Filho, E. L. Lim, M. L. Wong, and J. Licinio, Associations between adipokines and obesity-related cancer. Front Biosci (Landmark Ed). 16(5), p. 1634-50 (2011).
70. J. Park, T. S. Morley, M. Kim, D. J. Clegg, and P. E. Scherer, Obesity and cancer—mechanisms underlying tumour progression and recurrence. Nat Rev Endocrinol. 10(8), p. 455-465 (2014).
71. O. Sirin and M. G. Kolonin, Treatment of obesity as a potential complementary approach to cancer therapy. Drug Discov Today. 18(11-12), p. 567-73 (2013).
72. M. J. Khandekar, P. Cohen, and B. M. Spiegelman, Molecular mechanisms of cancer development in obesity. Nat Rev Cancer. 11(12), p. 886-95 (2011).
73. D. M. Huffman, M. S. Johnson, A. Watts, A. Elgavish, I. A. Eltoum, and T. R. Nagy, Cancer progression in the transgenic adenocarcinoma of mouse prostate mouse is related to energy balance, body mass, and body composition, but not food intake. Cancer Res. 67(1), p. 417-24 (2007).
74. Y. Zhang, A. C. Daquinag, F. Amaya-Manzanares, O. Sirin, C. Tseng, and M. G. Kolonin, Stromal progenitor cells from endogenous adipose tissue contribute to pericytes and adipocytes that populate the tumor microenvironment. Cancer Res. 72(20), p. 5198-208 (2012).
75. Y. Y. Wang, C. Attane, D. Milhas, B. Dirat, S. Dauvillier, A. Guerard, J. Gilhodes, I. Lazar, N. Alet, V. Laurent, S. Le Gonidec, D. Biard, C. Herve, F. Bost, G. S. Ren, F. Bono, G. Escourrou, M. Prentki, L. Nieto, P. Valet, and C. Muller, Mammary adipocytes stimulate breast cancer invasion through metabolic remodeling of tumor cells. JCI Insight. 2(4), p. e87489 (2017).
76. F. Rohrig and A. Schulze, The multifaceted roles of fatty acid synthesis in cancer. Nat Rev Cancer. 16(11), p. 732-749 (2016).
77. S. Beloribi-Djefaflia, S. Vasseur, and F. Guillaumond, Lipid metabolic reprogramming in cancer cells. Oncogenesis. 5, p. e189 (2016).
78. H. Ye, B. Adane, N. Khan, T. Sullivan, M. Minhajuddin, M. Gasparetto, B. Stevens, S. Pei, M. Balys, J. M. Ashton, D. J. Klemm, C. M. Woolthuis, A. W. Stranahan, C. Y. Park, and C. T. Jordan, Leukemic Stem Cells Evade Chemotherapy by Metabolic Adaptation to an Adipose Tissue Niche. Cell Stem Cell. 19(1), p. 23-37 (2016).
79. A. M. Cypess, Reassessing Human Adipose Tissue. N Engl J Med. 386(8), p. 768-779 (2022).
80. P. G. Cordeiro, Breast reconstruction after surgery for breast cancer. N Engl J Med. 359(15), p. 1590-601 (2008).
81. M. Juntunen, S. Heinonen, H. Huhtala, A. Rissanen, J. Kaprio, K. Kuismanen, K. H. Pietilainen, S. Miettinen, and M. Patrikoski, Evaluation of the effect of donor weight on adipose stromal/stem cell characteristics by using weight-discordant monozygotic twin pairs. Stem Cell Res Ther. 12(1), p. 516 (2021).
82. D. T. Chu, T. Nguyen Thi Phuong, N. L. B. Tien, D. K. Tran, L. B. Minh, V. V. Thanh, P. Gia Anh, V. H. Pham, and V. Thi Nga, Adipose Tissue Stem Cells for Therapy: An Update on the Progress of Isolation, Culture, Storage, and Clinical Application. J Clin Med. 8(7)(2019).
83. J. G. Doench, N. Fusi, M. Sullender, M. Hegde, E. W. Vaimberg, K. F. Donovan, I. Smith, Z. Tothova, C. Wilen, R. Orchard, H. W. Virgin, J. Listgarten, and D. E. Root, Optimized sgRNA design to maximize activity and minimize off-target effects of CRISPR-Cas9. Nat Biotechnol. 34(2), p. 184-191 (2016).
84. N. Matharu, S. Rattanasopha, S. Tamura, L. Maliskova, Y. Wang, A. Bernard, A. Hardin, W. L. Eckalbar, C. Vaisse, and N. Ahituv, CRISPR-mediated activation of a promoter or enhancer rescues obesity caused by haploinsufficiency. Science. 363(6424)(2019).
85. J. Schindelin, I. Arganda-Carreras, E. Frise, V. Kaynig, M. Longair, T. Pietzsch, S. Preibisch, C. Rueden, S. Saalfeld, B. Schmid, J. Y. Tinevez, D. J. White, V. Hartenstein, K. Eliceiri, P. Tomancak, and A. Cardona, Fiji: an open-source platform for biological-image analysis. Nat Methods. 9(7), p. 676-82 (2012).
86. W. Qiu and G. H. Su, Development of orthotopic pancreatic tumor mouse models. Methods Mol Biol. 980, p. 215-23 (2013).
87. J. M. Rosenbluth, R. C. J. Schackmann, G. K. Gray, L. M. Selfors, C. M. Li, M. Boedicker, H. J. Kuiken, A. Richardson, J. Brock, J. Garber, D. Dillon, N. Sachs, H. Clevers, and J. S. Brugge, Organoid cultures from normal and cancer-prone human breast tissues preserve complex epithelial lineages. Nat Commun. 11(1), p. 1711 (2020).
88. G. K. Gray, C. M. Li, J. M. Rosenbluth, L. M. Selfors, N. Girnius, J. R. Lin, R. C. J. Schackmann, W. L. Goh, K. Moore, H. K. Shapiro, S. Mei, K. D'Andrea, K. L. Nathanson, P. K. Sorger, S. Santagata, A. Regev, J. E. Garber, D. A. Dillon, and J. S. Brugge, A human breast atlas integrating single-cell proteomics and transcriptomics. Dev Cell. 57(11), p. 1400-1420 e7 (2022).

### Example 2

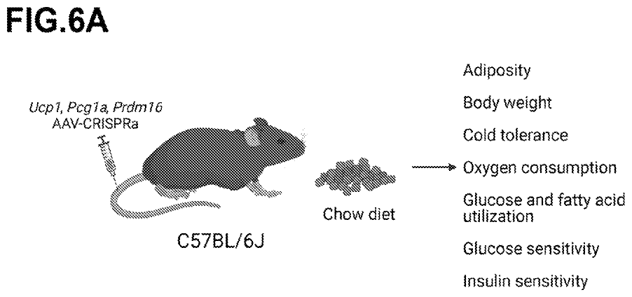

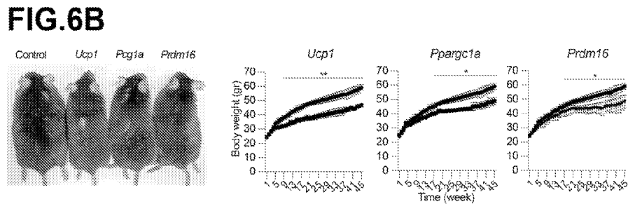

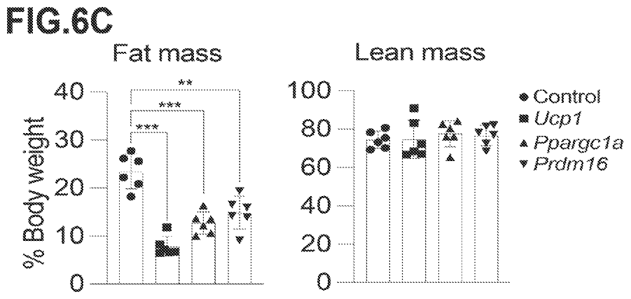

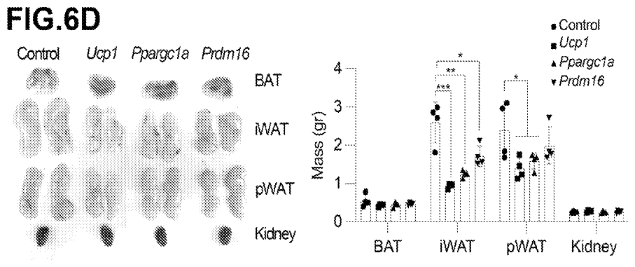

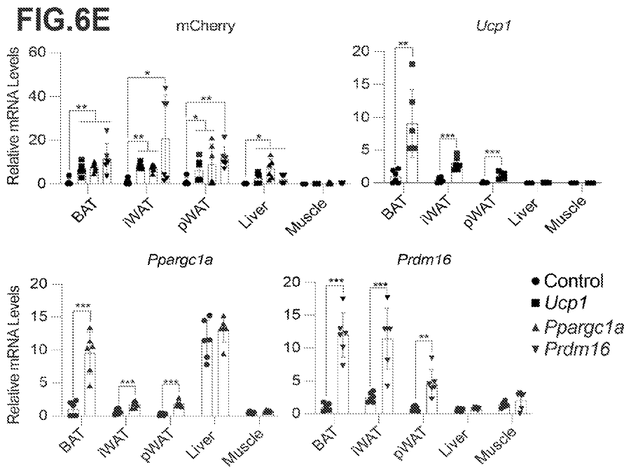

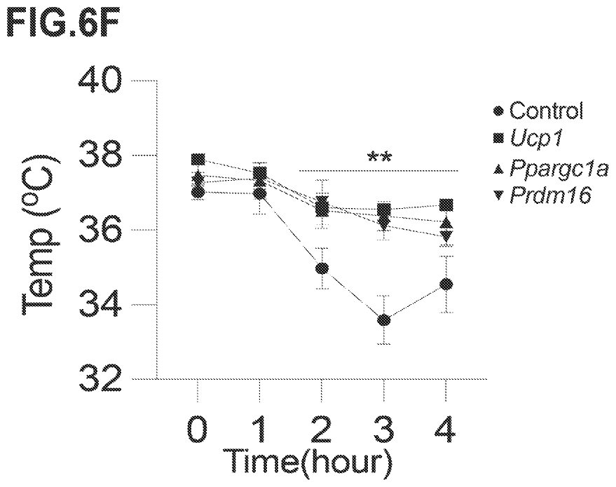

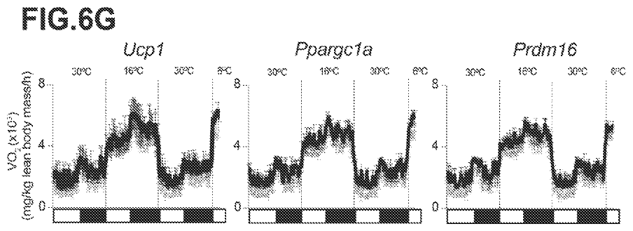

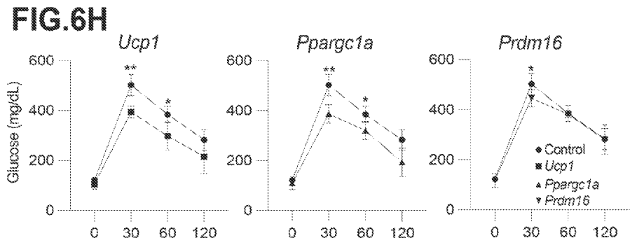

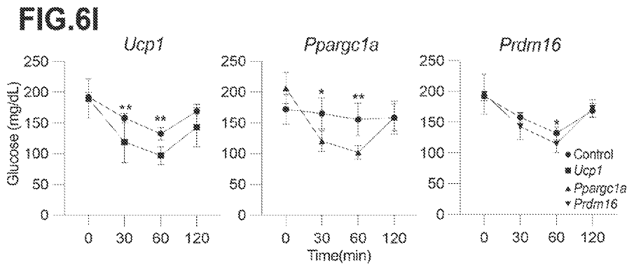

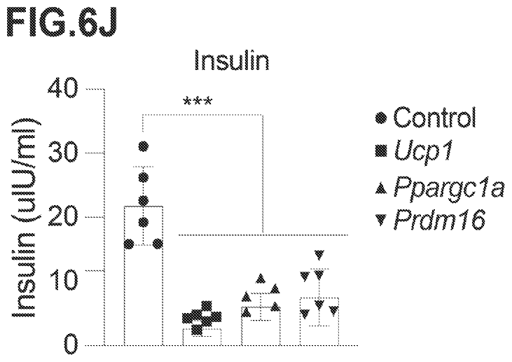

To examine whether CRISPRa-mediated upregulation of Ucp1, Ppargc1a, and Prdm16 can protect mice against obesity, we intravenously injected AAV-sadCas9-VP64 and AAV-gRNAs targeting Ucp1, Pcg1a, and Prdm16 to 4 week-old C57BL/6J mice, which are widely used for diet-induced obesity due to having many loci implicated in regulation of body weight, body fat, and obesity, and being susceptible to diet-induced obesity (FIG. 6A). Five weeks post injection, all Ucp1-, Ppargc1a-, and Prdm16-gRNAs treated mice began to gain weight less than control only AAV-sadCas9-VP64 injected mice and their body weights continued to be significantly lower than the control mice up to nearly one-year post-treatment (FIG. 6B). Using DEXA (dual energy X-ray absorptiometry), we found Ucp1-, Ppargc1a-, and Prdm16-AAV-CRISPRa mice to have a 30-60% lower fat mass than control mice with no significant difference in lean mass (FIG. 6C).

After 45 weeks, upon tissue dissection, all AAV-CRISPRa treated mice had smaller white fat pads, such as iWAT (inguinal) and pWAT (perigonadal) compared to control mice (FIG. 6D). Gene analysis indicated that mCherry (marker in sgRNA AAV) was increased in brown adipose tissue (BAT), iWAT, pWAT, and liver (FIG. 6E). Moreover, Ucp1, Ppargc1a, and Prdm16 mRNA levels were significantly higher only in adipose depots with the highest levels in BAT (FIG. 6E). We next examined cold tolerance in these mice by maintaining them at 4° C. for six hours after being at 30° C. (thermoneutrality). At 20 weeks, mice treated with Ucp1, Ppargc1a, and Prdm16 gRNAs were able to maintain their body temperature, while dCas9-VP64 only control mice had a decrease of ~5° C. after three hours of cold exposure (FIG. 6F). Moreover, the whole-body oxygen consumption rate (OCR) of mice with CRISPRa for all three genes, compared to control mice, as measured by the Comprehensive Lab Animal Monitoring System (CLAMS; Columbus Instruments) was significantly higher during both day and night when mice were kept at thermoneutrality of 30° C. and 16° C. This difference in OCR was more apparent when mice were exposed to cold at 4° C. (FIG. 6G). At 32 weeks, we also tested for glucose tolerance (GTT) and insulin sensitivity (ITT) in these mice. For GTT, mice were fasted for 24 hours prior to glucose administration (2 g/kg). For ITT, mice were fasted four hours prior to insulin (0.75 U/kg). Blood glucose was measured at 30, 60, and 120 minutes after the injections. All AAV-CRISPRa treated mice showed significantly higher glucose clearance than negative controls (dCas9-VP64 only) at all time points (FIG. 6H). Similarly, the CRISPRa treated mice had significantly higher insulin sensitivity than control mice (FIG. 6I). In addition, these mice showed lowered insulin levels in plasma, further indicating improved insulin response in AAV-CRISPRa treated mice (FIG. 6J). Overall, the results strongly demonstrate our CRISPRa targeting Ucp1, Ppargc1a, and Prdm16 reduce weigh gain and adiposity with increased energy expenditure and cold tolerance in mice.

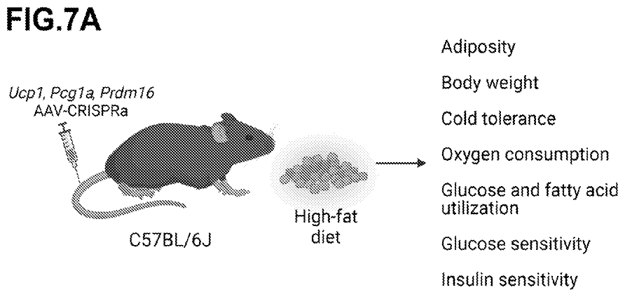

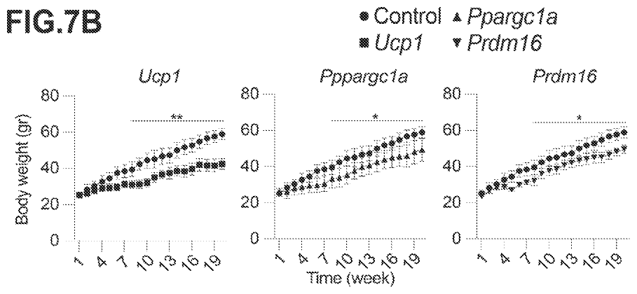

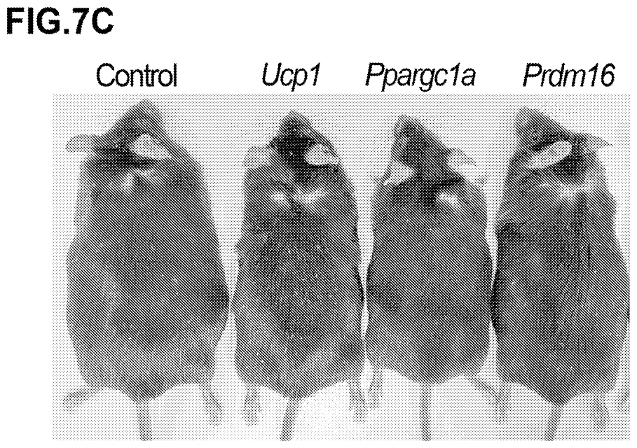

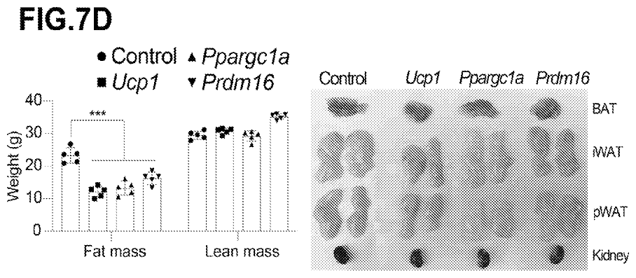

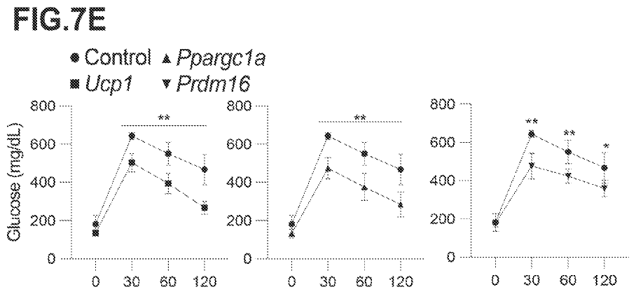

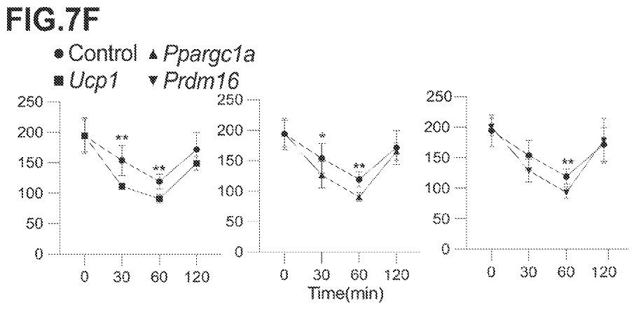

We next examined whether CRISPRa induced browning can be an effective treatment for high fat diet-induced obesity. We intravenously injected AAV-sadCas9-VP64 and AAV-gRNAs targeting Ucp1, Pparcg1a, and Prdm16 to 4-week-old C57BL/6J mice that were fed with high fat diet (60% fat) (FIG. 7A). After ten weeks, the AAV-CRISPRa-treated mice showed significantly reduced body weight compared to the dCas-VP64 control mice (FIG. 7B). These mice were also leaner (FIG. 7C) and showed decreased fat mass measured by DEXA (FIG. 7D). Upon dissection, we found AAV-CRISPRa mice had much smaller white fat pads, including iWAT and pWAT (FIG. 7D), suggesting our treatment reduced adiposity induced by HFD. When subjecting these mice to GTT, all CRISPRa treated mice showed significantly higher glucose clearance than negative control at all time points (FIG. 7E). Similarly, the CRISPRa treated mice had significantly higher insulin sensitivity than control mice (FIG. 7F). In summary, our CRISPRa treatment targeting Ucp1, Pcg1a, and Prdm16 protected mice against HFD-induced obesity and improved insulin sensitivity and glucose tolerance.

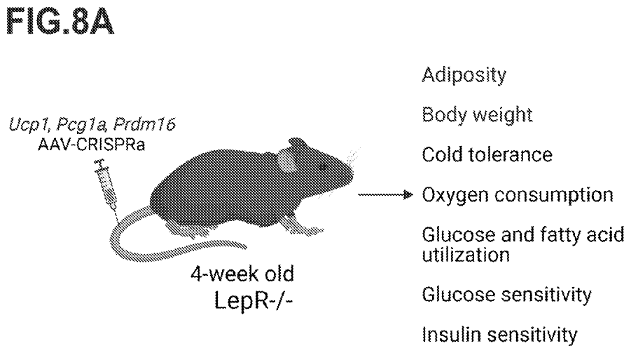

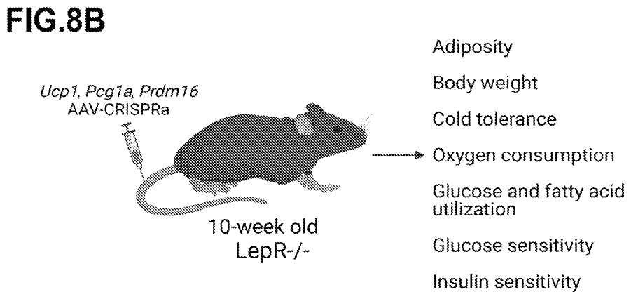

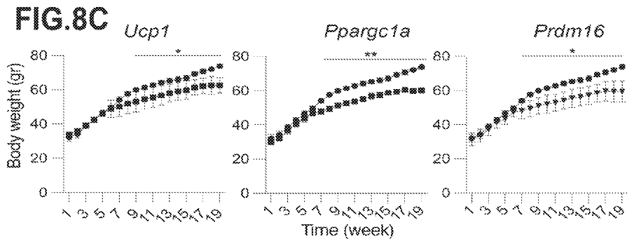

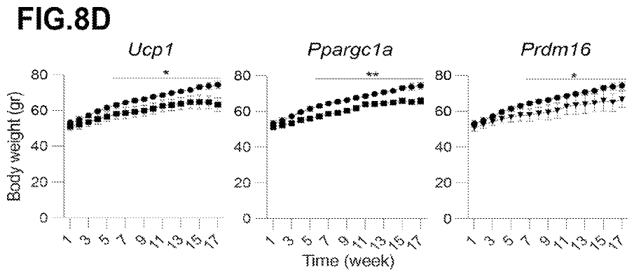

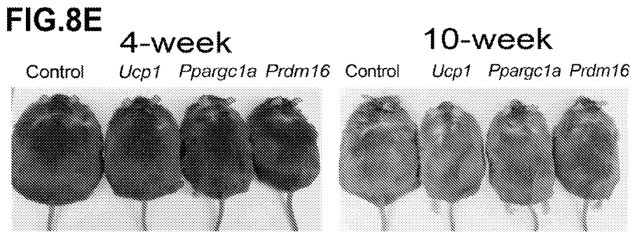

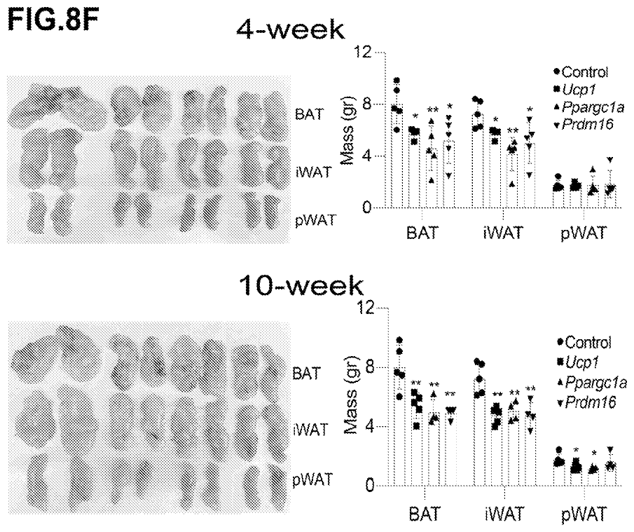

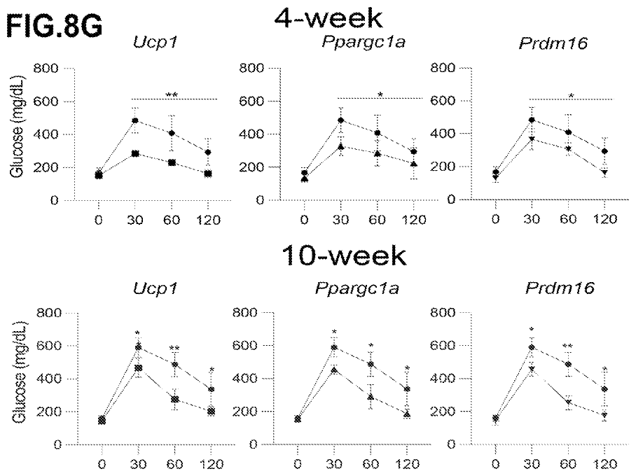

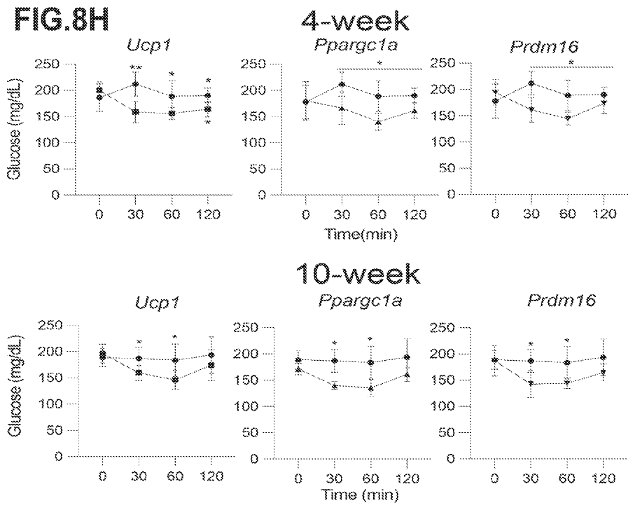

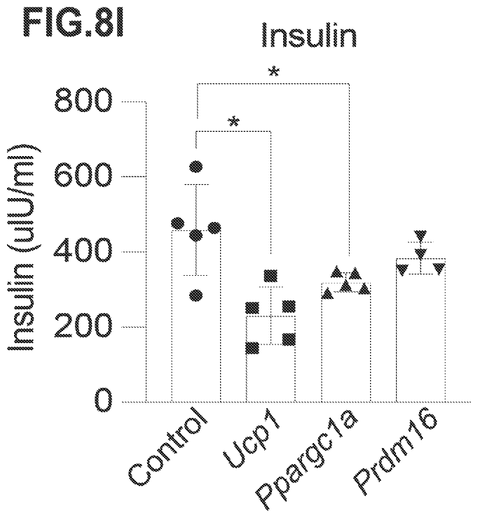

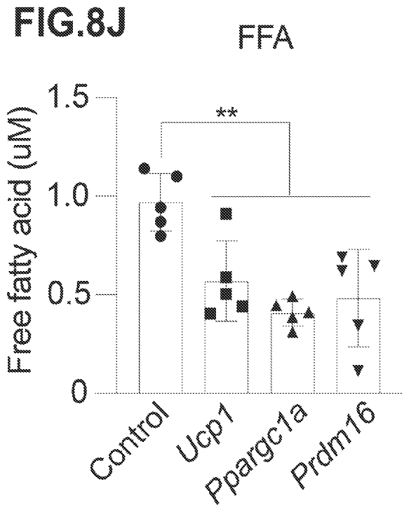

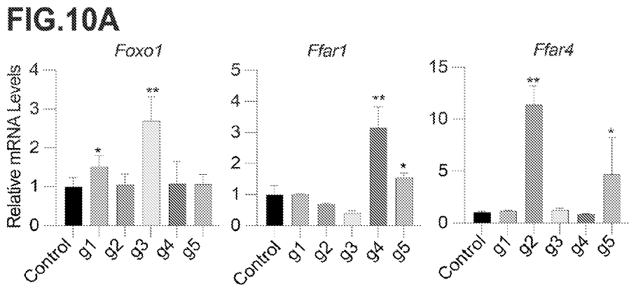

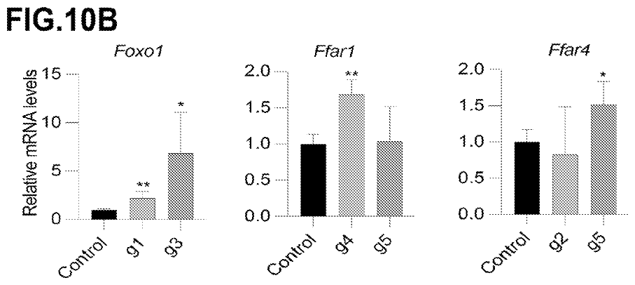

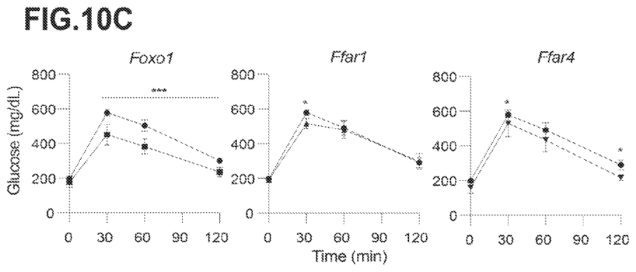

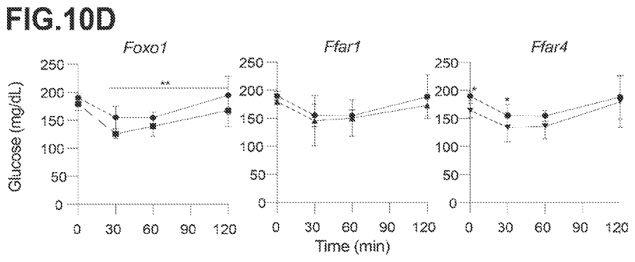

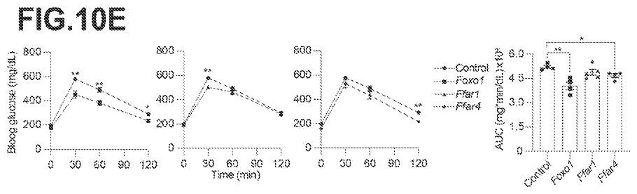

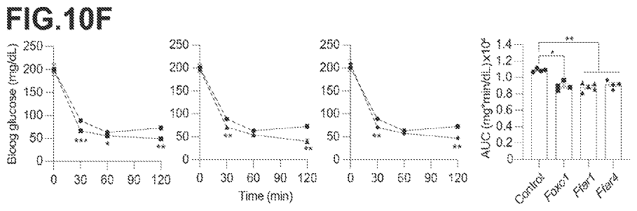

To further examine whether CRISPRa induced browning can be an effective obesity therapeutic, we tested its ability to rescue a commonly used obesity mouse model, ob/ob. In this model, mice are homozygous for a spontaneous loss-of-function mutation in the leptin (Lep) gene, leading to severe obesity, hyperphagia, hyperglycemia, glucose intolerance, and elevated plasma insulin1. We intravenously injected AAV-sadCas9-VP64 and AAV-gRNAs targeting Ucp1, Pparcg1a, and Prdm16 to 4 wk-old ob/ob mice (FIG. 8A) or 10 wk-old ob/ob mice which were morbidly obese (FIG. 8B). Following eight weeks, mice treated with AAV-CRISPRa at four weeks or 10 weeks showed significantly lower body weight than the dCas-VP64 control mice (FIG. 8C,D). These mice were also leaner (FIG. 8D) and showed reduced fat mass measured by DEXA (FIG. 8F). They also had significantly smaller BAT, iWAT and pWAT (FIG. 8F), suggesting our treatment reduced their leptin depletion associated obesity. When subjecting these mice to GTT, all AAV-CRISPRa treated mice showed significantly higher glucose clearance than negative control at all time points (FIG. 8G) and had significantly higher insulin sensitivity than control mice (FIG. 8H). All AAV-CRISPRa-treated mice had lower insulin and free fatty acid plasma levels, indicating improved metabolism. These data clearly indicate CRISPRa treatment not only prevents and alleviates obesity but also improves insulin sensitivity and glucose tolerance in ob/ob mice.

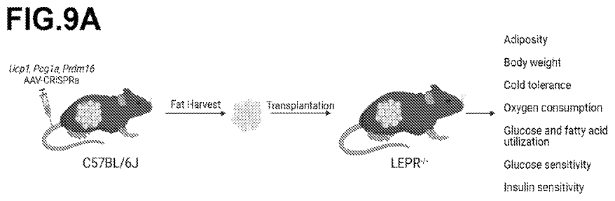

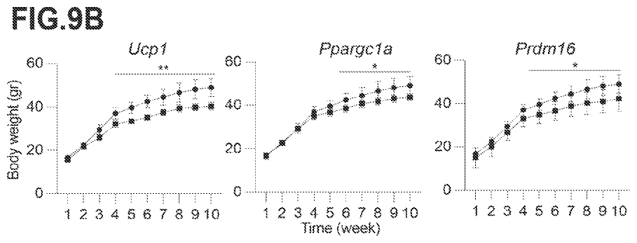

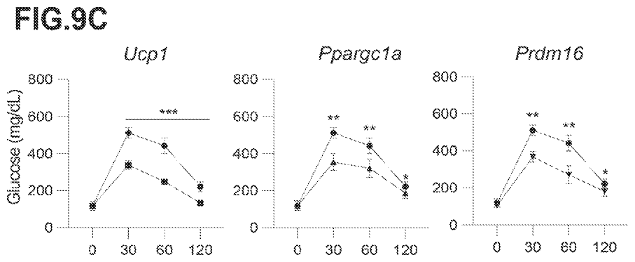

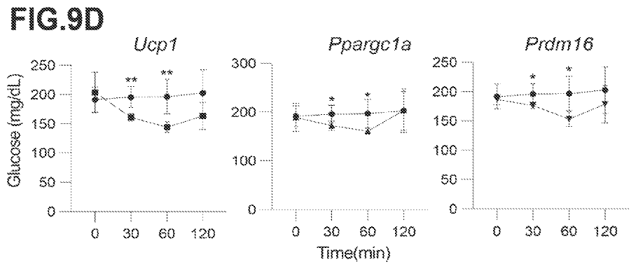

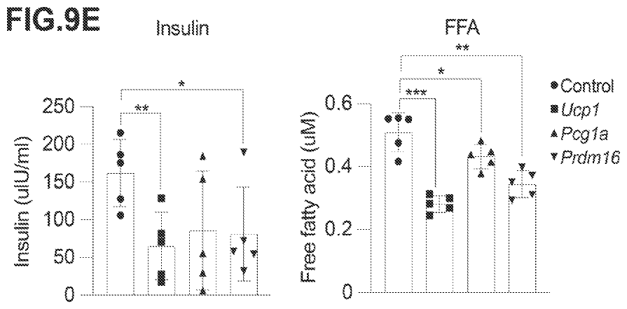

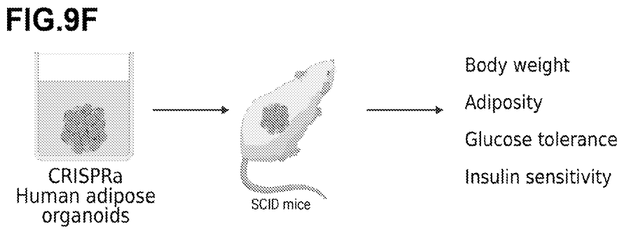

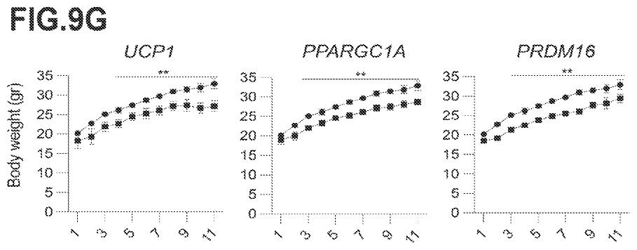

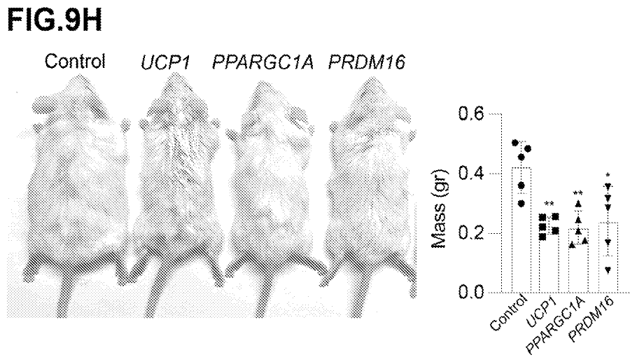

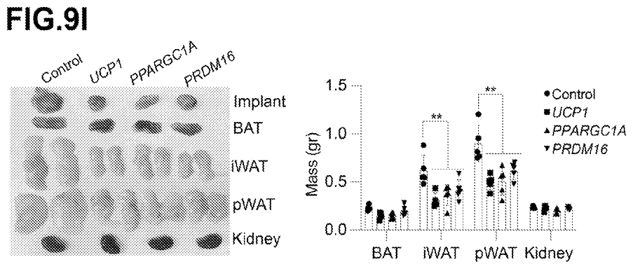

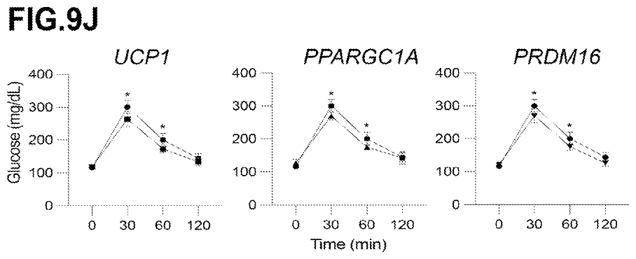

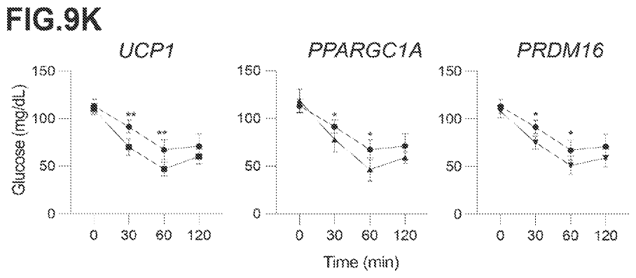

Due to its successful engraftment and long-term survival, fat transplantation is commonly used in many surgical procedures and has progressively increased the interest for therapeutic treatments². Using the patient's own adipose tissue for fat grafting also eliminates tissue rejection. Hence, here set out to utilize CRISPRa BAT-activated fat transplantation, as a novel obesity therapeutic. We first collected fat pad from C57BL/6J mice that were injected via tail-vein with AAV-CRISPRa targeting Ucp1, Pparcg1a, or Prdm16. After six weeks, mice were sacrificed and iWAT were collected, washed with cold PBS, minced into smaller pieces and implanted subcutaneously into five-week-old C57BL/6J mice (FIG. 9A). After five weeks, mice implanted with adipose of AAV-CRISPRa mice showed lower body weight than control dCas9-VP64 implanted mice (FIG. 9B). In addition, these mice had significantly reduced glucose tolerance and increased insulin response (FIG. 9C,D). Moreover, these CRISPRa-treated mice had lower plasma insulin and free fatty acid (FIG. 9E)

We next tested our CRISPRa therapy in human adipocytes. We utilized adipose organoids since they resemble human adipose tissue and recapitulate responses to endogenous stimuli providing proper tissue microenvironments that prevent further tissue dysfunction³. To culture 3D organoids, we plated 10⁵ cells in Nunc 96 well plates treated with Nunclon Delta in the same MSC basal (low serum) media supplemented with 5 ng/ml recombinant human EGF and FGF. Organoids formed after 48 hours and were then differentiated to adipose organoids using a differentiation cocktail containing IBMX, dexamethasone, insulin, T3, and rosiglitazone. Adipocytes formed 21-days post differentiation. The organoids were then infected with AAV9-based CRISPRa targeting the UCP1, PPARGC1a and PRDM16 promoters. Following 72 hours, we transplanted these human adipose organoids into immune deficient SCID mice (Jackson Lab #001303) to prevent organoid rejection. Each mouse was subcutaneously injected with 20 organoids mixed with Matrigel (FIG. 9F). The mice with CRISPRa treatment showed significantly lower body weight gain compared to control mice transplanted with dCas9-VP64 only treated organoids already three weeks post injection (FIG. 9G,H). Furthermore, these mice had smaller white fat pads, including iWAT and pWAT (FIG. 9I). When subjecting to GTT and ITT, CRISPRa organoid transplanted mice displayed higher glucose tolerance and insulin response than control mice (FIG. 9J-K). Combined, these data demonstrate our ability to generate human adipose organoids, manipulate their gene expression, transplant them in mice and utilize them to significantly reduce body weight in mice.

### REFERENCES

1. Zhang, Y. et al. Positional cloning of the mouse obese gene and its human homologue. Nature 372, 425-432 (1994).
2. Shukla, L., Yuan, Y., Shayan, R., Greening, D. W. & Karnezis, T. Fat therapeutics: The clinical capacity of adipose-derived stem cells and exosomes for human disease and tissue regeneration. Front. Pharmacol. 11, 158 (2020).
3. McCarthy, M. et al. Fat-on-A-chip models for research and discovery in obesity and its metabolic comorbidities. Tissue Eng. Part B Rev. 26, 586-595 (2020).

### Example 3

To examine whether CRISPRa-mediated upregulation of Ucp1, Ppargc1a, and Prdm16 can protect mice against diabetes, we first examined the therapeutic potential of our CRISPRa treatment in mice. We used C57BL/6J mice, widely used for diet-induced obesity due to having many loci implicated in regulation of body weight, body fat, and obesity, and being susceptible to diet-induced obesity. We optimized gRNA targeting the promoters of these genes in mice. We designed five gRNAs for Foxo1, Ffar1, and Ffar4 in mice using CRISPick. Guides along with dCas9-VP64 were transfected into differentiated 3T3-L1 adipocytes. Foxo1, Ffar1, and Ffar4 mRNA expression was measured by qRT-PCR. Several guides significantly increased the expression levels of these genes in these cells (FIG. 10A). The top two gRNA for each gene were selected for AAV serotype 9 (AAV9) packaging and used to infect 3T3-L1 adipocytes with these viruses along with AAV9 dCas9-VP64 finding gRNAs for all three genes that significantly increased expression compared to dCas9-VP64 only (FIG. 10B). Next, mice were tail-vein injected with AAV9-dCas9-VP64 and gRNA-AAV9 viruses targeting Foxo1, Ffar1, and Ffar4 individually. Mice were then fed with either chow or high fat diet (HFD). After five weeks, we performed glucose tolerance test and insulin tolerance test. For GTT, mice were fasted for 24 hours prior to glucose administration (2 g/kg). For ITT, mice were fasted four hours prior to insulin (0.75 U/kg). Blood glucose was measured at 30, 60, and 120 minutes after the injections. For mice on chow diet, all AAV-CRISPRa treated mice on either chow diet or HFD showed significantly higher glucose clearance than negative controls (dCas9-VP64 only) at all time points (FIG. 10C-E). In addition, these mice showed improved insulin response on either chow diet or HFD. Overall, the results strongly demonstrate our CRISPRa targeting Foxo1, Ffar1, and Ffar4 can improve insulin signaling in mice.

### Example 4

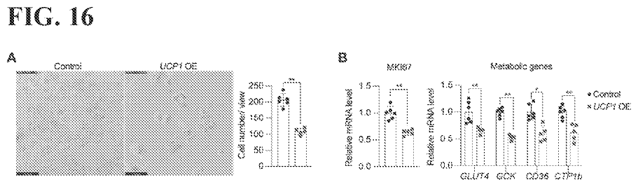

It was determined that an AAV9 containing a mammalian cDNA vector expressing UCP1 can express this gene in human adipocytes and suppress MCF7 cancer cell growth. FIG. 16A depicts representative images of MCF7 breast cancer cells that were co-cultured with UCP1-overexpressing human adipocytes. FIG. 16B depicts qRT-PCR of MKI67, a proliferative marker, and metabolic genes, including GLUT4, GCK, CD36, and CTP1b.

### Example 5

We show using breast organoids from dissected breast tissue of BRCA1/BRCA2/RAD51D mutation carriers that our therapeutic approach can suppress proliferation of premalignant cells for this high-risk group of patients, indicating an effective preventative measure for patients that are at high risk for breast cancer.

### UCP1-CRISPRa Adipocytes Suppress Proliferation in Breast Tissues at High Risk of Cancer Development

To examine whether UCP1-CRISPRa mammary adipocytes might prevent cancer development in individuals at high lifetime risk of cancer, we co-cultured them alongside breast organoids from dissected patient-matched breast tissue of BRCA1/BRCA2/RAD51D mutation carriers. We obtained three samples dissected from individual donors carrying a BRCA1 mutation (ORG158), a BRCA2 mutation (ORG150), and a RAD51D mutation (ORG164), as shown in Table S4. In all three cases, we observed that breast organoids co-cultured with donor matched UCP1-CRISPRa adipose organoids were significantly smaller and had lower organoid numbers compared to the negative control. In addition, we found that BRCA1/BRCA2 breast organoids co-cultured with UCP1-CRISPRa adipose organoids have lower expression levels of MKI67 and MTOR, known proliferation markers compared to controls. Furthermore, these breast organoids exhibited reduced expression levels of CK5, and CK17, suggesting reduced basal-luminal phenotypes in these BRCA1/BRCA2/RAD51D-heterozygous luminal progenitor-predominant breast cells (Shalabi, S. F. et al. Nat Aging 1, 838-849, (2021); Gray, G. K. et al. Dev Cell 57, 1400-1420 e1407, (2022)). In summary, our results indicate that UCP1-CRISPRa breast adipocytes inhibits premalignant phenotypes in breast organoids from donors with inherited cancer predisposition syndromes.

TABLE S4
Breast organoids each carrying a gene mutation.

| | | Germline | | |
|---|---|---|---|---|
| Name | Gene | mutation | Cancer history | Source of adipocytes |
| ORG150 | BRCA2 | 6174delT | Two breast cancers | ad-6 (matched breast) |
| ORG158 | BRCA1 | c.5266dup | Ductal carcinoma | ad-7 (matched breast) |
| | | | in situ (DCIS) | |
| ORG164 | RAD51D | c.270_271dupTA | Breast cancer | ad-8 (matched breast) |

### Example 6

We generated CRISPRa tetracycline inducible vectors and showed that they can turn on UCP1-CRISPRa by tetracyline treatment and suppress cancer growth in an inducible manner.

### Inducible or Cell Scaffold AMT Systems Suppress Cancer Progression

To further enhance the translation capabilities of AMT, we developed inducible AAV vectors designed to turn on dCas9-VP64 upon tetracycline treatment, thereby enabling to tightly regulate UCP1 expression in adipocytes or adipose organoids. We cloned the reverse tetracycline-control transactivator (rtTA) downstream of the CMV promoter into the gRNA-AAV vector and the tetracycline response element (TRE) and a minimal promoter upstream of dCas9-VP64 in the dCas9-VP64 AAV vector. Upon tetracycline treatment, rtTA binds to TRE and induces expression of dCas9-VP64. To first test this system, we infected TRE-dCas9-VP64-AAV with or without the rtTA-UCP1-gRNA-AAV, followed by the addition of DMSO or doxycycline to human adipocytes. We found that only cells in which both TRE-dCas9-VP64-AAV and rtTA UCP1-gRNA-AAV were transduced and treated with doxycycline had increased expression levels of dCas9-VP64 and UCP1. We next generated MCF7 xenografts in SCID mice and after four weeks implanted inducible UCP1-CRISPRa human adipose organoids. Following adipose organoid implantation, mice were fed with doxycycline diet for 3 weeks and tumors were subsequently dissected and examined. We found that tumors co-implanted with the inducible UCP1-CRISPRa human adipose organoids were significantly smaller than ones co-implanted with dCas9-VP64-treated adipose organoids. These tumors also had lower expression of the proliferation marker, MKI67, glycolytic genes (GLUT4 and GCK) and fatty oxidation genes (CD36 and CPT1b).

### Example 7

We implanted UCP1-CRISPRa adipose organoids in cell scaffolds that were generated for these organoids and show that implanting these scaffolds with breast cancer xenograft significantly suppresses tumor growth.

To further showcase the clinical applicability of AMT, we inserted UCP1-CRISPRa human adipose organoids into a polymer microwell scaffold, generated from polycaprolactone (PCL), and implanted it alongside tumors. The microwell scaffold organoid delivery platform is made from biodegradable polyester polycaprolactone (PCL) and manufactured using a microfabrication process involving photolithograhy and micromolding (Nyitray, C. E., Chavez, M. G. & Desai, T. A. Tissue Eng Part A 20, 1888-1895, (2014); Jeong, G. S. et al. Nat Commun 7, 11269, (2016); Girgin, M. U. et al. Nat Commun 12, 5140, (2021)). We made PCL scaffolds patterned with arrays of cylindrical microwells (Wise, K. D. & Najafi, K. Science (New York, N.Y.) 254, 1335-1342, (1991); Leong, T. G., Zarafshar, A. M. & Gracias, D. H. Small 6, 792-806, (2010); Steedman, M. R., Tao, S. L., Klassen, H. & Desai, T. A. Biomed Microdevices 12, 363-369 (2010); Kharbikar, B. N., Kumar, S. H., Kr, S. & Srivastava, R. Vol. 9668 256-272 (SPIE, 2015); Bernards, D. A. et al. ACS Materials Au 3, 255-264, (2023)). The microwells present a controlled three-dimensional (3D) microenvironment that provides a supportive niche for survival, function and enhanced integration of transplanted organoids (Girgin, M. U. et al. Nat Commun 12, 5140 (2021); Kharbikar, B. N., Chendke, G. S. & Desai, T. A. Adv Drug Deliv Rev 174, 87-113, (2021); Kharbikar, B. N., Mohindra, P. & Desai, T. A. Cell Stem Cell 29, 692-721 (2022)). Here, we manufactured microwells of 500 μm diameter, specifically designed to accommodate and spatially organize adipose organoids for transplantation. Using scanning electron microscopy, we found that adipose organoids attached to the microwell scaffold with filopodia and lamellipodia, suggesting that the implant is stable and can be extracted after implantation without leaving residual organoids. We next generated MCF7 xenografts in SCID mice for 4 weeks, loaded UCP1-CRISPRa and dCas9-VP64 only (negative control) human adipose organoids onto the device and co-implanted them next to the xenograft. Following three weeks, tumors were collected and examined, finding that tumors co-implanted with the scaffold-containing UCP1-CRISPRa human adipose organoids were significantly smaller than tumors co-implanted with the control scaffold adipose organoids containing dCas9-VP64 only. Furthermore, these tumors had lower expression levels of the proliferation marker, MKI67, glycolytic genes (GLUT4 and GCK) and fatty oxidation genes (CD36 and CPT1b). Combined, these data show that AMT could be used in an inducible manner or delivered via an integrated cell-scaffold delivery platform that can be readily removed or replaced to fit the changing tumor metabolic landscape.

### Example 8

To highlight the ability to customize this therapeutic approach for different cancer-associated metabolic programs, we upregulated uridine phosphorylase (UPP1) in human adipocyte/adipose organoids and show that they can suppress pancreatic ductal adenocarcinoma (PDA) cell line and xenografts growth. This cancer type is known to utilize uridine-derived ribose in glucose-restricted conditions (Nwosu, volume 618, pages 151-158 (2023). n this experiment, we are not making ‘brown’ adipocytes/adipose organoids, but rather making them utilize more uridine.

### UPP1-CRISPRa Adipose Organoids Suppress Pancreatic Ductal Adenocarcinoma

To examine whether AMT can be utilized to treat other cancer-associated metabolic pathways, we upregulated uridine phosphorylase (UPP1) in human adipocyte/adipose organoids and tested whether they can suppress pancreatic ductal adenocarcinoma (PDA), which is known to utilize uridine-derived ribose in glucose-restricted conditions³⁵. Using CRISPick36, we designed five gRNAs to upregulate UPP1 expression in human adipocytes and found all gRNAs were able to induce UPP1 expression in these cells. We next generated AAV9 virus for the top two gRNAs and transduced human adipocytes with AAV9 and found both gRNAs to significantly induced UPP1 expression. We used the top activating gRNA AAV for all subsequent experiments. We next transduced human adipocytes with UPP1-CRISPRa AAV9 and incubated these cells with 3H-labeled-uridine. We found these adipocytes to significantly uptake uridine during a 12-hour incubation and have increased lactate levels, a byproduct of uridine catabolism. We next co-cultured a PDA cell line, PANC-1, with UPP1-CRISPRa or dCas9-VP64 only (negative control) human adipocytes on top of transwell plates. Cells were cultured in low glucose-containing media with or without excess amount of uridine. We found that PANC-1 cells that were co-cultured with the UPP1-CRISPRa adipocytes had a significantly lower number of cells compared to the ones co-cultured with dCas9-VP64 adipocytes. This growth suppression was abolished with the addition of uridine. PANC-1 cells that were co-cultured with UPP1-CRISPRa adipocytes had lower ATP levels compared to control PANC-1 cells. In contrast, with an excess amount of uridine, there was no apparent difference in ATP levels between PANC-1 cells with UPP1-CRISPRa adipocytes and control cells. Furthermore, PANC-1 cells that were co-cultured with UPP1-CRISPRa modulated adipocytes had lower lactate and NADH levels than control cells. We next carried out a similar study but growing the cells in a high-glucose-containing media, finding that the growth suppression of PANC-1 growth by UPP1-CRISPRa adipocytes was reduced. Combined, these assays indicate that UPP1-CRISPRa adipocytes have increased uridine utilization and can outcompete PDA for uridine utilization in low glucose conditions.

We next set out to test whether UPP1-CRISPRa can suppress PDA xenograft growth. We implanted PANC-1 cells in SCID mice for four weeks, followed by the implantation of UPP1-CRISPRa and dCas9-VP64 only (negative control) adipose organoids. After three weeks, tumors were dissected and examined. We found that tumors co-implanted with UPP1-CRISPRa human adipose organoids were significantly smaller than ones co-implanted with dCas9-VP64 adipose organoids. In addition, these tumors had lower expression of MKI67, a proliferation marker. All the byproducts of uridine catabolism, including NADH and lactate were found to be reduced in tumors co-implanted with UPP1-CRISPRa human adipose organoids. In summary, these results suggest that CRISPRa upregulation of UPP1 in adipocytes/adipose organoids reduces uridine in the TME of PDA and leads to cancer growth suppression. Taken together, we demonstrate the clinical versatility and capacity of AMT to target various cancer metabolic pathways.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

### Informal Sequence Listing

| Seq. ID. No | Name | Sequence |
|---|---|---|
| Seq. ID. No: 1 | UCP1 gRNA-1 | CCCGAGGCACCGAGCGAGAAT |
| Seq. ID. No: 2 | UCP1 gRNA-2 | GCAGGGCTCCCGAGGCACCGA |
| Seq. ID. No: 3 | UCP1 gRNA-3 | AGGGCTCCCGAGGCACCGAGC |
| Seq. ID. No: 4 | UCP1 gRNA-4 | ACCGAGCGAGAATGGGAATGG |
| Seq. ID. No: 5 | UCP1 gRNA-5 | GCACCGAGCGAGAATGGGAAT |
| Seq. ID. No: 6 | PPARGC1a gRNA-1 | CACTGAAGCAGAGGGCTGCCT |
| Seq. ID. No: 7 | PPARGC1a gRNA-2 | TTAGAGCAGCAAGCTGCACAG |
| Seq. ID. No: 8 | PPARGC1a gRNA-3 | GTTAGAGCAGCAAGCTGCACA |
| Seq. ID. No: 9 | PPARGC1a gRNA-4 | GGGCTGCCTTTGAGTGACGTC |
| Seq. ID. No: 10 | PPARGC1a gRNA-5 | AGTTAGAGCAGCAAGCTGCAC |
| Seq. ID. No: 11 | PRDM16 gRNA-1 | CGGCGGCGGCGGCGCGACGAT |
| Seq. ID. No: 12 | PRDM16 gRNA-2 | GGCGGCGCGACGATGAGGATG |
| Seq. ID. No: 13 | PRDM16 gRNA-3 | GCCGCCGCCGCCGCCTCGGCG |
| Seq. ID. No: 14 | PRDM16 gRNA-4 | CGCCGCCGCCGCCGCCTCGGC |
| Seq. ID. No: 15 | PRDM16 gRNA-5 | GGCGGCGGCGGCGGCGCGACG |
| Seq. ID. No: 16 | FOXO1 gRNA-1 | CCGCCGCCTCCCCGTGGAAAA |
| Seq. ID. No: 17 | FOXO1 gRNA-2 | ATGGGCATGCGGCACCGCCGC |
| Seq. ID. No: 18 | FOXO1 gRNA-3 | GGGCAGCCAGTGGGCGCCGGG |
| Seq. ID. No: 19 | FOXO1 gRNA-4 | GCACCGCCGCCCGGGCAGCCA |
| Seq. ID. No: 20 | FOXO1 gRNA-5 | AGCCCGGCGCCCACTGGCTGC |
| Seq. ID. No: 21 | GPR40 gRNA-1 | TGGAGAATTACAATTCACCTG |
| Seq. ID. No: 22 | GPR40 gRNA-2 | GGTGAATTGTAATTCTCCACA |
| Seq. ID. No: 23 | GPR40 gRNA-3 | GGAGAATTACAATTCACCTGG |
| Seq. ID. No: 24 | GPR40 gRNA-4 | GCTGGAACCCGCGAGTGATCC |
| Seq. ID. No: 25 | GPR40 gRNA-5 | CTGGAACCCGCGAGTGATCCC |
| Seq. ID. No: 26 | GPR120 gRNA-1 | CAGTGCTGGCGCTCCTCAGAA |
| Seq. ID. No: 27 | GPR120 gRNA-2 | ACTGGGTGCAGGGAGCTGCAC |
| Seq. ID. No: 28 | GPR120 gRNA-3 | CCCTTCTGAGGAGCGCCAGCA |
| Seq. ID. No: 29 | GPR120 gRNA-4 | ACTTCCTGGTTCCCGCGCGCC |
| Seq. ID. No: 30 | GPR120 gRNA-5 | TGCTGGCGCTCCTCAGAAGGG |
| Seq. ID. No: 31 | GLUT4 gRNA-1 | CAAACTCTAAACCCCAGGCGG |
| Seq. ID. No: 32 | GLUT4 gRNA-2 | AGCCAAACTCTAAACCCCAGG |
| Seq. ID. No: 33 | GLUT4 gRNA-3 | AAACTCTAAACCCCAGGCGGA |
| Seq. ID. No: 34 | GLUT4 gRNA-4 | CCAAACTCTAAACCCCAGGCG |
| Seq. ID. No: 35 | GLUT4 gRNA-5 | TGGAGTTGGTGGGGCGGGCCA |
| Seq. ID. No: 36 | GLUT4 gRNA-6 | TGGCTGGCGAGACACGCCTC |
| Seq. ID. No: 37 | GLUT4 gRNA-7 | CCGGGATCCTAGCTGGTGGG |
| Seq. ID. No: 38 | GLUT4 gRNA-8 | GGTGGGCGGAGTCCTAACAC |
| Seq. ID. No: 39 | GLUT4 gRNA-9 | TTTAAGATGCTTCTGCAGCC |
| Seq. ID. No: 40 | GLUT4 gRNA-10 | CGACTGGGAAGTTTTGAGAA |
| Seq. ID. No: 41 | GLUT2 gRNA-1 | GGAGTTGGGCTACCCAGACCT |
| Seq. ID. No: 42 | GLUT2 gRNA-2 | GAGGAGTTGGGCTACCCAGAC |
| Seq. ID. No: 43 | GLUT2 gRNA-3 | CAGCGGCAGCTGAATATTGAT |
| Seq. ID. No: 44 | GLUT2 gRNA-4 | CTGCCGCTGAGAAGATTAGAC |
| Seq. ID. No: 45 | GLUT2 gRNA-5 | GTCTAATCTTCTCAGCGGCAG |
| Seq. ID. No: 46 | GLUT2 gRNA-6 | ACCGGGTTCTCACCTGGCGT |
| Seq. ID. No: 47 | GLUT2 gRNA-7 | GCCCCCACACCATAGGCCGA |
| Seq. ID. No: 48 | GLUT2 gRNA-8 | CCCGCCAGAGCTCCAGAGCA |
| Seq. ID. No: 49 | GLUT2 gRNA-9 | CCGCCCCTACTTTGCTCCAA |
| Seq. ID. No: 50 | GLUT2 gRNA-10 | TCTCCCTTCCCGTCTTTCCT |
| Seq. ID. No: 51 | CHREBP-gRNA-1 | CACCGGGTTCTCACCTGGCGT |
| Seq. ID. No: 52 | CHREBP-gRNA-2 | CACCATAGGCCGATCGGGTGG |
| Seq. ID. No: 53 | CHREBP-gRNA-3 | AGCATAATCCTTACGCCAGGT |
| Seq. ID. No: 54 | CHREBP-gRNA-4 | ACCCGCCAGAGCTCCAGAGCA |
| Seq. ID. No: 55 | CHREBP-gRNA-5 | CCCACACCATAGGCCGATCGG |
| Seq. ID. No: 56 | Linker | |

AAAAGGCCGGCGGCCACGAAAAAGGCCGGCCAGGCAAAAAAGAAAAA
GGGATCCGAGGCCAGCGGTTCCGGACGGGCT
Seq. ID. No: 57 UCP1 protein
MGGLTASDVHPTLGVQLFSAGIAACLADVITFPLDTAKVRLQVQGECPTSS
VIRYKGVLGTITAVVKTEGRMKLYSGLPAGLQRQISSASLRIGLYDTVQEFLTAGKET
APSLGSKILAGLTTGGVAVFIGQPTEVVKVRLQAQSHLHGIKPRYTGTYNAYRIIATT
EGLTGLWKGTTPNLMRSVIINCTELVTYDLMKEAFVKNNILADDVPCHLVSALIAGF
CATAMSSPVDVVKTRFINSPPGQYKSVPNCAMKVFTNEGPTAFFKGLVPSFLRLGSW
NVIMFVCFEQLKRELSKSRQTMDCAT
Seq. ID. No: 58 PPARGC1A
MAWDMCNQDSESVWSDIECAALVGEDQPLCPDLPELDLSELDVNDLDTDS
FLGGLKWCSDQSEIISNQYNNEPSNIFEKIDEENEANLLAVLTETLDSLPVDEDGLPSF
DALTDGDVTTDNEASPSSMPDGTPPPQEAEEPSLLKKLLLAPANTQLSYNECSGLSTQ
NHANHNHRIRTNPAIVKTENSWSNKAKSICQQQKPQRRPCSELLKYLTTNDDPPHTK
PTENRNSSRDKCTSKKKSHTQSQSQHLQAKPTTLSLPLTPESPNDPKGSPFENKTIERT
LSVELSGTAGLTPPTTPPHKANQDNPFRASPKLKSSCKTVVPPPSKKPRYSESSGTQG
NNSTKKGPEQSELYAQLSKSSVLTGGHEERKTKRPSLRLFGDHDYCQSINSKTEILINI
SQELQDSRQLENKDVSSDWQGQICSSTDSDQCYLRETLEASKQVSPCSTRKQLQDQE
IRAELNKHFGHPSQAVFDDEADKTGELRDSDFSNEQFSKLPMFINSGLAMDGLFDDS
EDESDKLSYPWDGTQSYSLFNVSPSCSSFNSPCRDSVSPPKSLFSQRPQRMRSRSRSFS
RHRSCSRSPYSRSRSRSPGSRSSSRSCYYYESSHYRHRTHRNSPLYVRSRSRSPYSRRP
RYDSYEEYQHERLKREEYRREYEKRESERAKQRERQRQKAIEERRVIYVGKIRPDTT
RTELRDRFEVFGEIEECTVNLRDDGDSYGFITYRYTCDAFAALENGYTLRRSNETDFE
LYFCGRKQFFKSNYADLDSNSDDFDPASTKSKYDSLDFDSLLKEAQRSLRR
Seq. ID. No: 59 PRDM16
MRSKARARKLAKSDGDVVNNMYEPNRDLLASHSAEDEAEDSAMSPIPVGP
PSPFPTSEDFTPKEGSPYEAPVYIPEDIPIPADFELRESSIPGAGLGVWAKRKMEAGERL
GPCVVVPRAAAKETDFGWEQILTDVEVSPQEGCITKISEDLGSEKFCVDANQAGAGS
WLKYIRVACSCDDQNLTMCQISEQIYYKVIKDIEPGEELLVHVKEGVYPLGTVPPGL
DEEPTFRCDECDELFQSKLDLRRHKKYTCGSVGAALYEGLAEELKPEGLGGGSGQA
HECKDCERMFPNKYSLEQHMVIHTEEREYKCDQCPKAFNWKSNLIRHQMSHDSGKR
FECENCVKVFTDPSNLQRHIRSQHVGARAHACPDCGKTFATSSGLKQHKHIHSTVKP
FICEVCHKSYTQFSNLCRHKRMHADCRTQIKCKDCGQMFSTTSSLNKHRRFCEGKN
HYTPGGIFAPGLPLTPSPMMDKAKPSPSLNHASLGFNEYFPSRPHPGSLPFSTAPPTFP
ALTPGFPGIFPPSLYPRPPLLPPTSLLKSPLNHTQDAKLPSPLGNPALPLVSAVSNSSQG
TTAAAGPEEKFESRLEDSCVEKLKTRSSDMSDGSDFEDVNTTTGTDLDTTTGTGSDL
DSDVDSDPDKDKGKGKSAEGQPKFGGGLAPPGAPNSVAEVPVFYSQHSFFPPPDEQL
LTATGAAGDSIKAIASIAEKYFGPGFMGMQEKKLGSLPYHSAFPFQFLPNFPHSLYPF
TDRALAHNLLVKAEPKSPRDALKVGGPSAECPFDLTTKPKDVKPILPMPKGPSAPAS
GEEQPLDLSIGSRARASQNGGGREPRKNHVYGERKLGAGEGLPQVCPARMPQQPPL
HYAKPSPFFMDPIYSRVEKRKVTDPVGALKEKYLRPSPLLFHPQMSAIETMTEKLESF
AAMKADSGSSLQPLPHHPFNFRSPPPTLSDPILRKGKERYTCRYCGKIFPRSANLTRHL
RTHTGEQPYRCKYCDRSFSISSNLQRHVRNIHNKEKPFKCHLCNRCFGQQTNLDRHL
KKHEHENAPVSQHPGVLTNHLGTSASSPTSESDNHALLDEKEDSYFSEIRNFIANSEM
NQASTRTEKRADMQIVDGSAQCPGLASEKQEDVEEEDDDDLEEDDEDSLAGKSQDD
TVSPAPEPQAAYEDEEDEEPAASLAVGFDHTRRCAEDHEGGLLALEPMPTFGKGLDL
RRAAEEAFEVKDVLNSTLDSEALKHTLCRQAKNQAYAMMLSLSEDTPLHTPSQGSL
DAWLKVTGATSESGAFHPINHL
Seq. ID. No: 60 FFAR1
MDLPPQLSFGLYVAAFALGFPLNVLAIRGATAHARLRLTPSLVYALNLGCS
DLLLTVSLPLKAVEALASGAWPLPASLCPVFAVAHFFPLYAGGGFLAALSAGRYLGA
AFPLGYQAFRRPCYSWGVCAAIWALVLCHLGLVFGLEAPGGWLDHSNTSLGINTPV
NGSPVCLEAWDPASAGPARFSLSLLLFFLPLAITAFCYVGCLRALARSGLTHRRKLRA
AWVAGGALLTLLLCVGPYNASNVASFLYPNLGGSWRKLGLITGAWSVVLNPLVTG
YLGRGPGLKTVCAARTQGGKSQK
Seq. ID. No: 61 FFAR4
MSPECARAAGDAPLRSLEQANRTRFPFFSDVKGDHRLVLAAVETTVLVLIF
AVSLLGNVCALVLVARRRRRGATACLVLNLFCADLLFISAIPLVLAVRWTEAWLLGP
VACHLLFYVMTLSGSVTILTLAAVSLERMVCIVHLQRGVRGPGRRARAVLLALIWG
YSAVAALPLCVFFRVVPQRLPGADQEISICTLIWPTIPGEISWDVSFVTLNFLVPGLVIV
ISYSKILQITKASRKRLTVSLAYSESHQIRVSQQDFRLFRTLFLLMVSFFIMWSPIIITILL
ILIQNFKQDLVIWPSLFFWVVAFTFANSALNPILYNMTLCRNEWKKIFCCFWFPEKGA
ILTDTSVKRNDLSIISG
Seq. ID. No: 62 FOXO1
MAEAPQVVEIDPDFEPLPRPRSCTWPLPRPEFSQSNSATSSPAPSGSAAANPD
AAAGLPSASAAAVSADFMSNLSLLEESEDFPQAPGSVAAAVAAAAAAAATGGLCGD
FQGPEAGCLHPAPPQPPPPGPLSQHPPVPPAAAGPLAGQPRKSSSSRRNAWGNLSYA
DLITKAIESSAEKRLTLSQIYEWMVKSVPYFKDKGDSNSSAGWKNSIRHNLSLHSKFI
RVQNEGTGKSSWWMLNPEGGKSGKSPRRRAASMDNNSKFAKSRSRAAKKKASLQS
GQEGAGDSPGSQFSKWPASPGSHSNDDFDNWSTFRPRTSSNASTISGRLSPIMTEQDD
LGEGDVHSMVYPPSAAKMASTLPSLSEISNPENMENLLDNLNLLSSPTSLTVSTQSSP
GTMMQQTPCYSFAPPNTSLNSPSPNYQKYTYGQSSMSPLPQMPIQTLQDNKSSYGG
MSQYNCAPGLLKELLTSDSPPHNDIMTPVDPGVAQPNSRVLGQNVMMGPNSVMSTY
GSQASHNKMMNPSSHTHPGHAQQTSAVNGRPLPHTVSTMPHTSGMNRLTQVKTPV
QVPLPHPMQMSALGGYSSVSSCNGYGRMGLLHQEKLPSDLDGMFIERLDCDMESIIR
NDLMDGDTLDFNFDNVLPNQSFPHSVKTTTHSWVSG
Seq. ID. No: 63 SLC2A1
MEPSSKKLTGRLMLAVGGAVLGSLQFGYNTGVINAPQKVIEEFYNQTWVH
RYGESILPTTLTTLWSLSVAIFSVGGMIGSFSVGLFVNRFGRRNSMLMMNLLAFVSAV
LMGFSKLGKSFEMLILGRFIIGVYCGLTTGFVPMYVGEVSPTALRGALGTLHQLGIVV
GILIAQVFGLDSIMGNKDLWPLLLSIIFIPALLQCIVLPFCPESPRFLLINRNEENRAKSV
LKKLRGTADVTHDLQEMKEESRQMMREKKVTILELFRSPAYRQPILIAVVLQLSQQL
SGINAVFYYSTSIFEKAGVQQPVYATIGSGIVNTAFTVVSLFVVERAGRRTLHLIGLAG
MAGCAILMTIALALLEQLPWMSYLSIVAIFGFVAFFEVGPGPIPWFIVAELFSQGPRPA
AIAVAGFSNWTSNFIVGMCFQYVEQLCGPYVFIIFTVLLVLFFIFTYFKVPETKGRTFD
EIASGFRQGGASQSDKTPEELFHPLGADSQV
Seq. ID. No: 64 SLC2A2
MPSGFQQIGSEDGEPPQQRVTGTLVLAVFSAVLGSLQFGYNIGVINAPQKVI
EQSYNETWLGRQGPEGPSSIPPGTLTTLWALSVAIFSVGGMISSFLIGIISQWLGRKRA
MLVNNVLAVLGGSLMGLANAAASYEMLILGRFLIGAYSGLTSGLVPMYVGEIAPTH
LRGALGTLNQLAIVIGILIAQVLGLESLLGTASLWPLLLGLTVLPALLQLVLLPFCPESP
RYLYIIQNLEGPARKSLKRLTGWADVSGVLAELKDEKRKLERERPLSLLQLLGSRTH
RQPLIIAVVLQLSQQLSGINAVFYYSTSIFETAGVGQPAYATIGAGVVNTVFTLVSVLL
VERAGRRTLHLLGLAGMCGCAILMTVALLLLERVPAMSYVSIVAIFGFVAFFEIGPGP
IPWFIVAELFSQGPRPAAMAVAGFSNWTSNFIIGMGFQYVAEAMGPYVFLLFAVLLL
GFFIFTFLRVPETRGRTFDQISAAFHRTPSLLEQEVKPSTELEYLGPDEND
Seq. ID. No: 65 SLC2A3
MGTQKVTPALIFAITVATIGSFQFGYNTGVINAPEKIIKEFINKTLTDKGNAPP
SEVLLTSLWSLSVAIFSVGGMIGSFSVGLFVNRFGRRNSMLIVNLLAVTGGCFMGLC
KVAKSVEMLILGRLVIGLFCGLCTGFVPMYIGEISPTALRGAFGTLNQLGIVVGILVA
QIFGLEFILGSEELWPLLLGFTILPAILQSAALPFCPESPRFLLINRKEEENAKQILQRLW
GTQDVSQDIQEMKDESARMSQEKQVTVLELFRVSSYRQPIIISIVLQLSQQLSGINAVF
YYSTGIFKDAGVQEPIYATIGAGVVNTIFTVVSLFLVERAGRRTLHMIGLGGMAFCST
LMTVSLLLKDNYNGMSFVCIGAILVFVAFFEIGPGPIPWFIVAELFSQGPRPAAMAVA
GCSNWTSNFLVGLLFPSAAHYLGAYVFIIFTGFLITFLAFTFFKVPETRGRTFEDITRAF
EGQAHGADRSGKDGVMEMNSIEPAKETTTNV
Seq. ID. No: 66 SLC2A4
MPSGFQQIGSEDGEPPQQRVTGTLVLAVFSAVLGSLQFGYNIGVINAPQKVI
EQSYNETWLGRQGPEGPSSIPPGTLTTLWALSVAIFSVGGMISSFLIGIISQWLGRKRA
MLVNNVLAVLGGSLMGLANAAASYEMLILGRFLIGAYSGLTSGLVPMYVGEIAPTH
LRGALGTLNQLAIVIGILIAQVLGLESLLGTASLWPLLLGLTVLPALLQLVLLPFCPESP
RYLYIIQNLEGPARKSLKRLTGWADVSGVLAELKDEKRKLERERPLSLLQLLGSRTH
RQPLIIAVVLQLSQQLSGINAVFYYSTSIFETAGVGQPAYATIGAGVVNTVFTLVSVLL
VERAGRRTLHLLGLAGMCGCAILMTVALLLLERVPAMSYVSIVAIFGFVAFFEIGPGP
IPWFIVAELFSQGPRPAAMAVAGFSNWTSNFIIGMGFQYVAEAMGPYVFLLFAVLLL
GFFIFTFLRVPETRGRTFDQISAAFHRTPSLLEQEVKPSTELEYLGPDEND
Seq. ID. No: 67 MLXIPL
MAGALAGLAAGLQVPRVAPSPDSDSDTDSEDPSLRRSAGGLLRSQVIHSGH
FMVSSPHSDSLPRRRDQEGSVGPSDFGPRSIDPTLTRLFECLSLAYSGKLVSPKWKNF
KGLKLLCRDKIRLNNAIWRAWYIQYVKRRKSPVCGFVTPLQGPEADAHRKPEAVVL
EGNYWKRRIEVVMREYHKWRIYYKKRLRKPSREDDLLAPKQAEGRWPPPEQWCKQ
LFSSVVPVLLGDPEEEPGGRQLLDLNCFLSDISDTLFTMTQSGPSPLQLPPEDAYVGN
ADMIQPDLTPLQPSLDDFMDISDFFTNSRLPQPPMPSNFPEPPSFSPVVDSLFSSGTLGP
EVPPASSAMTHLSGHSRLQARNSCPGPLDSSAFLSSDFLLPEDPKPRLPPPPVPPPLLH
YPPPAKVPGLEPCPPPPFPPMAPPTALLQEEPLFSPRFPFPTVPPAPGVSPLPAPAAFPPT
PQSVPSPAPTPFPIELLPLGYSEPAFGPCFSMPRGKPPAPSPRGQKASPPTLAPATASPP
TTAGSNNPCLTQLLTAAKPEQALEPPLVSSTLLRSPGSPQETVPEFPCTFLPPTPAPTPP
RPPPGPATLAPSRPLLVPKAERLSPPAPSGSERRLSGDLSSMPGPGTLSVRVSPPQPILS
RGRPDSNKTENRRITHISAEQKRRFNIKLGFDTLHGLVSTLSAQPSLKVSKATTLQKT
AEYILMLQQERAGLQEEAQQLRDEIEELNAAINLCQQQLPATGVPITHQRFDQMRDM
FDDYVRTRTLHNWKFWVFSILIRPLFESFNGMVSTASVHTLRQTSLAWLDQYCSLPA
LRPTVLNSLRQLGTSTSILTDPGRIPEQATRAVTEGTLGKPL
Seq. ID. No: 68 CD36
MGCDRNCGLIAGAVIGAVLAVFGGILMPVGDLLIQKTIKKQVVLEEGTIAFK
NWVKTGTEVYRQFWIFDVQNPQEVMMNSSNIQVKQRGPYTYRVRFLAKENVTQDA
EDNTVSFLQPNGAIFEPSLSVGTEADNFTVLNLAVAAASHIYQNQFVQMILNSLINKS
KSSMFQVRTLRELLWGYRDPFLSLVPYPVTTTVGLFYPYNNTADGVYKVFNGKDNI
SKVAIIDTYKGKRNLSYWESHCDMINGTDAASFPPFVEKSQVLQFFSSDICRSIYAVFE
SDVNLKGIPVYRFVLPSKAFASPVENPDNYCFCTEKIISKNCTSYGVLDISKCKEGRPV
YISLPHFLYASPDVSEPIDGLNPNEEEHRTYLDIEPITGFTLQFAKRLQVNLLVKPSEKI
QVLKNLKRNYIVPILWLNETGTIGDEKANMFRSQVTGKINLLGLIEMILLSVGVVMF
VAFMISYCACRSKTIK
Seq. ID. No: 69 CPT1B
MAEAHQAVAFQFTVTPDGVDFRLSREALKHVYLSGINSWKKRLIRIKNGIL
RGVYPGSPTSWLVVIMATVGSSFCNVDISLGLVSCIQRCLPQGCGPYQTPQTRALLSM
AIFSTGVWVTGIFFFRQTLKLLLCYHGWMFEMHGKTSNLTRIWAMCIRLLSSRHPML
YSFQTSLPKLPVPRVSATIQRYLESVRPLLDDEEYYRMELLAKEFQDKTAPRLQKYL
VLKSWWASNYVSDWWEEYIYLRGRSPLMVNSNYYVMDLVLIKNTDVQAARLGNII
HAMIMYRRKLDREEIKPVMALGIVPMCSYQMERMENTTRIPGKDTDVLQHLSDSRH
VAVYHKGRFFKLWLYEGARLLKPQDLEMQFQRILDDPSPPQPGEEKLAALTAGGRV
EWAQARQAFFSSGKNKAALEAIERAAFFVALDEESYSYDPEDEASLSLYGKALLHGN
CYNRWFDKSFTLISFKNGQLGLNAEHAWADAPIIGHLWEFVLGTDSFHLGYTETGHC
LGKPNPALAPPTRLQWDIPKQCQAVIESSYQVAKALADDVELYCFQFLPFGKGLIKK
CRTSPDAFVQIALQLAHFRDRGKFCLTYEASMTRMFREGRTETVRSCTSESTAFVQA
MMEGSHTKADLRDLFQKAAKKHQNMYRLAMTGAGIDRHLFCLYLVSKYLGVSSPF
LAEVLSEPWRLSTSQIPQSQIRMFDPEQHPNHLGAGGGFGPVADDGYGVSYMIAGEN
TIFFHISSKFSSSETNAQRFGNHIRKALLDIADLFQVPKAYS
Seq. ID. No: 70 AIFM2
MGSQVSVESGALHVVIVGGGFGGIAAASQLQALNVPFMLVDMKDSFHHNV
AALRASVETGFAKKTFISYSVTFKDNFRQGLVVGIDLKNQMVLLQGGEALPFSHLIL
ATGSTGPFPGKFNEVSSQQAAIQAYEDMVRQVQRSRFIVVVGGGSAGVEMAAEIKTE
YPEKEVTLIHSQVALADKELLPSVRQEVKEILLRKGVQLLLSERVSNLEELPLNEYRE
YIKVQTDKGTEVATNLVILCTGIKINSSAYRKAFESRLASSGALRVNEHLQVEGHSNV
YAIGDCADVRTPKMAYLAGLHANIAVANIVNSVKQRPLQAYKPGALTFLLSMGRND
GVGQISGFYVGRLMVRLTKSRDLFVSTSWKTMRQSPP
Seq. ID. No: 71 GSK3B
MSGRPRTTSFAESCKPVQQPSAFGSMKVSRDKDGSKVTTVVATPGQGPDRP
QEVSYTDTKVIGNGSFGVVYQAKLCDSGELVAIKKVLQDKRFKNRELQIMRKLDHC
NIVRLRYFFYSSGEKKDEVYLNLVLDYVPETVYRVARHYSRAKQTLPVIYVKLYMY
QLFRSLAYIHSFGICHRDIKPQNLLLDPDTAVLKLCDFGSAKQLVRGEPNVSYICSRY
YRAPELIFGATDYTSSIDVWSAGCVLAELLLGQPIFPGDSGVDQLVEIIKVLGTPTREQ
IREMNPNYTEFKFPQIKAHPWTKVFRPRTPPEAIALCSRLLEYTPTARLTPLEACAHSF
FDELRDPNVKLPNGRDTPALFNFTTQELSSNPPLATILIPPHARIQAAASTPTNATAAS
DANTGDRGQTNNAASASASNST
Seq. ID. No: 72 IRS1
MASPPESDGFSDVRKVGYLRKPKSMHKRFFVLRAASEAGGPARLEYYENE
KKWRHKSSAPKRSIPLESCFNINKRADSKNKHLVALYTRDEHFAIAADSEAEQDSWY
QALLQLHNRAKGHHDGAAALGAGGGGGSCSGSSGLGEAGEDLSYGDVPPGPAFKE
VWQVILKPKGLGQTKNLIGIYRLCLTSKTISFVKLNSEAAAVVLQLMNIRRCGHSENF
FFIEVGRSAVTGPGEFWMQVDDSVVAQNMHETILEAMRAMSDEFRPRSKSQSSSNCS
NPISVPLRRHHLNNPPPSQVGLTRRSRTESITATSPASMVGGKPGSFRVRASSDGEGT
MSRPASVDGSPVSPSTNRTHAHRHRGSARLHPPLNHSRSIPMPASRCSPSATSPVSLSS
SSTSGHGSTSDCLFPRRSSASVSGSPSDGGFISSDEYGSSPCDFRSSFRSVTPDSLGHTP
PARGEEELSNYICMGGKGPSTLTAPNGHYILSRGGNGHRCTPGTGLGTSPALAGDEA
ASAADLDNRFRKRTHSAGTSPTITHQKTPSQSSVASIEEYTEMMPAYPPGGGSGGRLP
GHRHSAFVPTRSYPEEGLEMHPLERRGGHHRPDSSTLHTDDGYMPMSPGVAPVPSG
RKGSGDYMPMSPKSVSAPQQIINPIRRHPQRVDPNGYMMMSPSGGCSPDIGGGPSSSS
SSSNAVPSGTSYGKLWTNGVGGHHSHVLPHPKPPVESSGGKLLPCTGDYMNMSPVG
DSNTSSPSDCYYGPEDPQHKPVLSYYSLPRSFKHTQRPGEPEEGARHQHLRLSTSSGR
LLYAATADDSSSSTSSDSLGGGYCGARLEPSLPHPHHQVLQPHLPRKVDTAAQTNSR
LARPTRLSLGDPKASTLPRAREQQQQQQPLLHPPEPKSPGEYVNIEFGSDQSGYLSGP
VAFHSSPSVRCPSQLQPAPREEETGTEEYMKMDLGPGRRAAWQESTGVEMGRLGPA
PPGAASICRPTRAVPSSRGDYMTMQMSCPRQSYVDTSPAAPVSYADMRTGIAAEEVS
LPRATMAAASSSSAASASPTGPQGAAELAAHSSLLGGPQGPGGMSAFTRVNLSPNRN
QSAKVIRADPQGCRRRHSSETFSSTPSATRVGNTVPFGAGAAVGGGGGSSSSSEDVK
RHSSASFENVWLRPGELGGAPKEPAKLCGAAGGLENGLNYIDLDLVKDFKQCPQEC
TPEPQPPPPPPPHQPLGSGESSSTRRSSEDLSAYASISFQKQPEDRQ
Seq. ID. No: 73 DGAT1
MGDRGSSRRRRTGSRPSSHGGGGPAAAEEEVRDAAAGPDVGAAGDAPAPA
PNKDGDAGVGSGHWELRCHRLQDSLFSSDSGFSNYRGILNWCVVMLILSNARLFLE
NLIKYGILVDPIQVVSLFLKDPYSWPAPCLVIAANVFAVAAFQVEKRLAVGALTEQA
GLLLHVANLATILCFPAAVVLLVESITPVGSLLALMAHTILFLKLFSYRDVNSWCRRA
RAKAASAGKKASSAAAPHTVSYPDNLTYRDLYYFLFAPTLCYELNFPRSPRIRKRFLL
RRILEMLFFTQLQVGLIQQWMVPTIQNSMKPFKDMDYSRIIERLLKLAVPNHLIWLIF
FYWLFHSCLNAVAELMQFGDREFYRDWWNSESVTYFWQNWNIPVHKWCIRHFYKP
MLRRGSSKWMARTGVFLASAFFHEYLVSVPLRMFRLWAFTGMMAQIPLAWFVGRF
FQGNYGNAAVWLSLIIGQPIAVLMYVHDYYVLNYEAPAAEA
Seq. ID. No: 74 DGAT2
MKTLIAAYSGVLRGERQAEADRSQRSHGGPALSREGSGRWGTGSSILSALQ
DLFSVTWLNRSKVEKQLQVISVLQWVLSFLVLGVACSAILMYIFCTDCWLIAVLYFT
WLVFDWNTPKKGGRRSQWVRNWAVWRYFRDYFPIQLVKTHNLLTTRNYIFGYHPH
GIMGLGAFCNFSTEATEVSKKFPGIRPYLATLAGNFRMPVLREYLMSGGICPVSRDTI
DYLLSKNGSGNAIIIVVGGAAESLSSMPGKNAVTLRNRKGFVKLALRHGADLVPIYS
FGENEVYKQVIFEEGSWGRWVQKKFQKYIGFAPCIFHGRGLFSSDTWGLVPYSKPIT
TVVGEPITIPKLEHPTQQDIDLYHTMYMEALVKLFDKHKTKFGLPETEVLEVN
Seq. ID. No: 75 ACACA
MDEPSPLAQPLELNQHSRFIIGSVSEDNSEDEISNLVKLDLLEEKEGSLSPASV
GSDTLSDLGISSLQDGLALHIRSSMSGLHLVKQGRDRKKIDSQRDFTVASPAEFVTRF
GGNKVIEKVLIANNGIAAVKCMRSIRRWSYEMFRNERAIRFVVMVTPEDLKANAEYI
KMADHYVPVPGGPNNNNYANVELILDIAKRIPVQAVWAGWGHASENPKLPELLLK
NGIAFMGPPSQAMWALGDKIASSIVAQTAGIPTLPWSGSGLRVDWQENDFSKRILNV
PQELYEKGYVKDVDDGLQAAEEVGYPVMIKASEGGGGKGIRKVNNADDFPNLFRQ
VQAEVPGSPIFVMRLAKQSRHLEVQILADQYGNAISLFGRDCSVQRRHQKIIEEAPATI
ATPAVFEHMEQCAVKLAKMVGYVSAGTVEYLYSQDGSFYFLELNPRLQVEHPCTE
MVADVNLPAAQLQIAMGIPLYRIKDIRMMYGVSPWGDSPIDFEDSAHVPCPRGHVIA
ARITSENPDEGFKPSSGTVQELNFRSNKNVWGYFSVAAAGGLHEFADSQFGHCFSW
GENREEAISNMVVALKELSIRGDFRTTVEYLIKLLETESFQMNRIDTGWLDRLIAEKV
QAERPDTMLGVVCGALHVADVSLRNSVSNFLHSLERGQVLPAHTLLNTVDVELIYE
GVKYVLKVTRQSPNSYVVIMNGSCVEVDVHRLSDGGLLLSYDGSSYTTYMKEEVDR
YRITIGNKTCVFEKENDPSVMRSPSAGKLIQYIVEDGGHVFAGQCYAEIEVMKMVMT
LTAVESGCIHYVKRPGAALDPGCVLAKMQLDNPSKVQQAELHTGSLPRIQSTALRGE
KLHRVFHYVLDNLVNVMNGYCLPDPFFSSKVKDWVERLMKTLRDPSLPLLELQDIM
TSVSGRIPPNVEKSIKKEMAQYASNITSVLCQFPSQQIANILDSHAATLNRKSEREVFF
MNTQSIVQLVQRYRSGIRGHMKAVVMDLLRQYLRVETQFQNGHYDKCVFALREEN
KSDMNTVLNYIFSHAQVTKKNLLVTMLIDQLCGRDPTLTDELLNILTELTQLSKTTN
AKVALRARQVLIASHLPSYELRHNQVESIFLSAIDMYGHQFCIENLQKLILSETSIFDV
LPNFFYHSNQVVRMAALEVYVRRAYIAYELNSVQHRQLKDNTCVVEFQFMLPTSHP
NRGNIPTLNRMSFSSNLNHYGMTHVASVSDVLLDNSFTPPCQRMGGMVSFRTFEDF
VRIFDEVMGCFSDSPPQSPTFPEAGHTSLYDEDKVPRDEPIHILNVAIKTDCDIEDDRL
AAMFREFTQQNKATLVDHGIRRLTFLVAQKDFRKQVNYEVDRRFHREFPKFFTFRA
RDKFEEDRIYRHLEPALAFQLELNRMRNFDLTAIPCANHKMHLYLGAAKVEVGTEV
TDYRFFVRAIIRHSDLVTKEASFEYLQNEGERLLLEAMDELEVAFNNTNVRTDCNHIF
LNFVPTVIMDPSKIEESVRSMVMRYGSRLWKLRVLQAELKINIRLTPTGKAIPIRLFLT
NESGYYLDISLYKEVTDSRTAQIMFQAYGDKQGPLHGMLINTPYVTKDLLQSKRFQA
QSLGTTYIYDIPEMFRQSLIKLWESMSTQAFLPSPPLPSDMLTYTELVLDDQGQLVHM
NRLPGGNEIGMVAWKMTFKSPEYPEGRDIIVIGNDITYRIGSFGPQEDLLFLRASELAR
AEGIPRIYVSANSGARIGLAEEIRHMFHVAWVDPEDPYKGYRYLYLTPQDYKRVSAL
NSVHCEHVEDEGESRYKITDIIGKEEGIGPENLRGSGMIAGESSLAYNEIITISLVTCRAI
GIGAYLVRLGQRTIQVENSHLILTGAGALNKVLGREVYTSNNQLGGIQIMHNNGVTH
CTVCDDFEGVFTVLHWLSYMPKSVHSSVPLLNSKDPIDRIIEFVPTKTPYDPRWMLA
GRPHPTQKGQWLSGFFDYGSFSEIMQPWAQTVVVGRARLGGIPVGVVAVETRTVEL
SIPADPANLDSEAKIIQQAGQVWFPDSAFKTYQAIKDFNREGLPLMVFANWRGFSGG
MKDMYDQVLKFGAYIVDGLRECCQPVLVYIPPQAELRGGSWVVIDSSINPRHMEMY
ADRESRGSVLEPEGTVEIKFRRKDLVKTMRRVDPVYIHLAERLGTPELSTAERKELEN
KLKEREEFLIPIYHQVAVQFADLHDTPGRMQEKGVISDILDWKTSRTFFYWRLRRLLL
EDLVKKKIHNANPELTDGQIQAMLRRWFVEVEGTVKAYVWDNNKDLAEWLEKQL
TEEDGVHSVIEENIKCISRDYVLKQIRSLVQANPEVAMDSIIHMTQHISPTQRAEVIRIL
STMDSPST
Seq. ID. No: 76 ACACB
MVLLLCLSCLIFSCLTFSWLKIWGKMTDSKPITKSKSEANLIPSQEPFPASDN
SGETPQRNGEGHTLPKTPSQAEPASHKGPKDAGRRRNSLPPSHQKPPRNPLSSSDAAP
SPELQANGTGTQGLEATDTNGLSSSARPQGQQAGSPSKEDKKQANIKRQLMTNFILG
SFDDYSSDEDSVAGSSRESTRKGSRASLGALSLEAYLTTGEAETRVPTMRPSMSGLH
LVKRGREHKKLDLHRDFTVASPAEFVTRFGGDRVIEKVLIANNGIAAVKCMRSIRRW
AYEMFRNERAIRFVVMVTPEDLKANAEYIKMADHYVPVPGGPNNNNYANVELIVDI
AKRIPVQAVWAGWGHASENPKLPELLCKNGVAFLGPPSEAMWALGDKIASTVVAQ
TLQVPTLPWSGSGLTVEWTEDDLQQGKRISVPEDVYDKGCVKDVDEGLEAAERIGF
PLMIKASEGGGGKGIRKAESAEDFPILFRQVQSEIPGSPIFLMKLAQHARHLEVQILAD
QYGNAVSLFGRDCSIQRRHQKIVEEAPATIAPLAIFEFMEQCAIRLAKTVGYVSAGTV
EYLYSQDGSFHFLELNPRLQVEHPCTEMIADVNLPAAQLQIAMGVPLHRLKDIRLLY
GESPWGVTPISFETPSNPPLARGHVIAARITSENPDEGFKPSSGTVQELNFRSSKNVWG
YFSVAATGGLHEFADSQFGHCFSWGENREEAISNMVVALKELSIRGDFRTTVEYLIN
LLETESFQNNDIDTGWLDYLIAEKVQAEKPDIMLGVVCGALNVADAMFRTCMTDFL
HSLERGQVLPADSLLNLVDVELIYGGVKYILKVARQSLTMFVLIMNGCHIEIDAHRL
NDGGLLLSYNGNSYTTYMKEEVDSYRITIGNKTCVFEKENDPTVLRSPSAGKLTQYT
VEDGGHVEAGSSYAEMEVMKMIMTLNVQERGRVKYIKRPGAVLEAGCVVARLELD
DPSKVHPAEPFTGELPAQQTLPILGEKLHQVFHSVLENLTNVMSGFCLPEPVFSIKLKE
WVQKLMMTLRHPSLPLLELQEIMTSVAGRIPAPVEKSVRRVMAQYASNITSVLCQFP
SQQIATILDCHAATLQRKADREVFFINTQSIVQLVQRYRSGIRGYMKTVVLDLLRRYL
RVEHHFQQAHYDKCVINLREQFKPDMSQVLDCIFSHAQVAKKNQLVIMLIDELCGPD
PSLSDELISILNELTQLSKSEHCKVALRARQILIASHLPSYELRHNQVESIFLSAIDMYG
HQFCPENLKKLILSETTIFDVLPTFFYHANKVVCMASLEVYVRRGYIAYELNSLQHRQ
LPDGTCVVEFQFMLPSSHPNRMTVPISITNPDLLRHSTELFMDSGFSPLCQRMGAMV
AFRRFEDFTRNFDEVISCFANVPKDTPLFSEARTSLYSEDDCKSLREEPIHILNVSIQCA
DHLEDEALVPILRTFVQSKKNILVDYGLRRITFLIAQEKEFPKFFTFRARDEFAEDRIY
RHLEPALAFQLELNRMRNFDLTAVPCANHKMHLYLGAAKVKEGVEVTDHRFFIRAII
RHSDLITKEASFEYLQNEGERLLLEAMDELEVAFNNTSVRTDCNHIFLNFVPTVIMDP
FKIEESVRYMVMRYGSRLWKLRVLQAEVKINIRQTTTGSAVPIRLFITNESGYYLDISL
YKEVTDSRSGNIMFHSFGNKQGPQHGMLINTPYVTKDLLQAKRFQAQTLGTTYIYDF
PEMFRQALFKLWGSPDKYPKDILTYTELVLDSQGQLVEMNRLPGGNEVGMVAFKM
RFKTQEYPEGRDVIVIGNDITFRIGSFGPGEDLLYLRASEMARAEGIPKIYVAANSGAR
IGMAEEIKHMFHVAWVDPEDPHKGFKYLYLTPQDYTRISSLNSVHCKHIEEGGESRY
MITDIIGKDDGLGVENLRGSGMIAGESSLAYEEIVTISLVTCRAIGIGAYLVRLGQRVI
QVENSHIILTGASALNKVLGREVYTSNNQLGGVQIMHYNGVSHITVPDDFEGVYTILE
WLSYMPKDNHSPVPIITPTDPIDREIEFLPSRAPYDPRWMLAGRPHPTLKGTWQSGFF
DHGSFKEIMAPWAQTVVTGRARLGGIPVGVIAVETRTVEVAVPADPANLDSEAKIIQ
QAGQVWFPDSAYKTAQAVKDFNREKLPLMIFANWRGFSGGMKDMYDQVLKFGAY
IVDGLRQYKQPILIYIPPYAELRGGSWVVIDATINPLCIEMYADKESRGGVLEPEGTVE
IKFRKKDLIKSMRRIDPAYKKLMEQLGEPDLSDKDRKDLEGRLKAREDLLLPIYHQV
AVQFADFHDTPGRMLEKGVISDILEWKTARTFLYWRLRRLLLEDQVKQEILQASGEL
SHVHIQSMLRRWFVETEGAVKAYLWDNNQVVVQWLEQHWQAGDGPRSTIRENITY
LKHDSVLKTIRGLVEENPEVAVDCVIYLSQHISPAERAQVVHLLSTMDSPAST
Seq. ID. No: 77 NRIP1
MTHGEELGSDVHQDSIVLTYLEGLLMHQAAGGSGTAVDKKSAGHNEEDQ
NFNISGSAFPTCQSNGPVLNTHTYQGSGMLHLKKARLLQSSEDWNAAKRKRLSDSIM
NLNVKKEALLAGMVDSVPKGKQDSTLLASLLQSFSSRLQTVALSQQIRQSLKEQGYA
LSHDSLKVEKDLRCYGVASSHLKTLLKKSKVKDQKPDTNLPDVTKNLIRDRFAESPH
HVGQSGTKVMSEPLSCAARLQAVASMVEKRASPATSPKPSVACSQLALLLSSEAHLQ
QYSREHALKTQNANQAASERLAAMARLQENGQKDVGSYQLPKGMSSHLNGQARTS
SSKLMASKSSATVFQNPMGIIPSSPKNAGYKNSLERNNIKQAANNSLLLHLLKSQTIP
KPMNGHSHSERGSIFEESSTPTTIDEYSDNNPSFTDDSSGDESSYSNCVPIDLSCKHRTE
KSESDQPVSLDNFTQSLLNTWDPKVPDVDIKEDQDTSKNSKLNSHQKVTLLQLLLGH
KNEENVEKNTSPQGVHNDVSKFNTQNYARTSVIESPSTNRTTPVSTPPLLTSSKAGSPI
NLSQHSLVIKWNSPPYVCSTQSEKLTNTASNHSMDLTKSKDPPGEKPAQNEGAQNSA
TFSASKLLQNLAQCGMQSSMSVEEQRPSKQLLTGNTDKPIGMIDRLNSPLLSNKTNA
VEENKAFSSQPTGPEPGLSGSEIENLLERRTVLQLLLGNPNKGKSEKKEKTPLRDEST
QEHSERALSEQILMVKIKSEPCDDLQIPNTNVHLSHDAKSAPFLGMAPAVQRSAPALP
VSEDFKSEPVSPQDFSFSKNGLLSRLLRQNQDSYLADDSDRSHRNNEMALLESKNLC
MVPKKRKLYTEPLENPFKKMKNNIVDAANNHSAPEVLYGSLLNQEELKFSRNDLEF
KYPAGHGSASESEHRSWARESKSFNVLKQLLLSENCVRDLSPHRSNSVADSKKKGH
KNNVTNSKPEFSISSLNGLMYSSTQPSSCMDNRTFSYPGVVKTPVSPTFPEHLGCAGS
RPESGLLNGCSMPSEKGPIKWVITDAEKNEYEKDSPRLTKTNPILYYMLQKGGNSVT
SRETQDKDIWREASSAESVSQVTAKEELLPTAETKASFFNLRSPYNSHMGNNASRPH
SANGEVYGLLGSVLTIKKESE
Seq. ID. No: 78 UCP1 promoter
CCAGCTGTGTGACCTTGGGCAAGTGATGAATCTGCTCTGTGTTCAGTTTT
CCCATCTGCCAAGGGATAATGGTACTAATACCCTTCCAGTTATAATGTAT
CTGAAGCACTTAGAACAATGTCTGGTTCATAGTTAAGCCCTCAAATAAAT
GCTACCTAAAGTGACAGGAATTCTTAGAGTGCACTGGCACTCAGGAAATA
TGTTTCCTTTTCTTTTTCCCCTAAACAAACTCCCTGCAGCATTCGTCCAC
TTTTTCTGCTCTGGTCTTTCCTGGACCTGATGAGGAACCTCTTAAGAGGA
AATATGTCTTTTCCTGTCCTTTCAACTCTCTTCACAAAGTCTGGCTTTCT
TTGCTTCAAACAGAAGGCTTTGAACTTTGTTGCATACACAAATGATGAGG
GAAGCTTGTGGAAAGACACTCTGGTCTCTAGCCTGAGGATTCAGACTCAG
AGGACCATGGTAGTCCTCGTTTGGATTTCTGACAAGCTCCATAGATGTTT
CTGATATACTTCCCTCTTTGGAGAACTTTAAGTTAAACATTTTTCTGTAG
CCCACCCATATTCTCCAGATGGAGATCCAAACTGGGCACAAAACTTCCAT
GACTGGGTGACCTTTACAAGTATATATCTGCATCTAAAAAATCGATTTTG
CATACTTTTTTCTATGAACAATGAGCTAATGTTTGGGGAAGGGAGAGTTC
AAATATGAATGTTTTCTCGCCAATTTGTTATGAAAACCACTCCTCTGTGT
GTTACACTTTTTGGAACAGAGCGGAGCCCAAGGCTTTTCTGCCCCTCCCA
GGAACGCTCACGGCTTACTTTCTTTCCCTGCGGTGAGGAACTCCTGGACC
GTGTCGTAGAGGCCGATCCTGAGAGAGGCGGAGCTGATTTGCCGCTGAAG
CCCCGCAGGCAGCCCGCTGTAGAGTTTCATCCGCCCTTCTGTTTTTACCA
CAGCGGTGATTGTTCCCAGGACACCTTTATACCTAATAACACTGGACGTC
GGGCATTCACCTTGGACCTGGAAATAAGAAAGGTGCAGAACAGAAGGGAG
CGAAATTGAGTTCACTCTTAAGCCAAGATTTCCCCCTGTGTTCCTATTTT
CCGTTTCTTTTCTCAGCAACATTCCTCTTCCATCCACCTCCCTCTACCGT
GTGTGTGTGTGTGTGTGTGTGTGTGTGTGTGTGTGTGTGTGTGTGTTCGC
TCACGCGCAGATGGAGGGAGGCTGGCTACAATGCACATTTTGAATATCCC
CCTCCCCAGGAACTCTCCAGAACCCCTAGGGCAGTAGGATAGGACAGAAC
CCCCGAAGAATCAAAGAAAGGAAAAAGCACCTGACTAGTAAACTCAGGAG
AGGCCAATATTCTCAGCTCTTAAAATACTAAGTGCACCCTAAATTTTGGG
AGAATTTGGTAGTAGTCATGGTCAACCCATTCTCTGTGGCCCTGGTGGTT
CTTCAGCTAATCCCAGAAAAAGTTCCAGTTTCCCATCCTAAAGCACCAGC
CCTGGGACAAGGCCAGACTGCTTTGGAAGGTCACCTACCCTACCTAGAAA
CAGTGGTTACACCAAAGCAGAGAATAAATGGAATGCACGCAACAGCGTCC
CCTCTGTCCTGAGACCCCTTGTCTTACCCCTCTGCCTAGCTACCTGGAGC
CGGACTTTGGCCGTGTCCAGCGGGAAGGTGATCACGTCCGCCAAGCACGC
CGCTATTCCAGCTGAGAAGAGCTGGACCCCCAGGGTCGGGTGTACGTCCG
AGGCTGTCAGGCCCCCCATCTTCACTCAGAGACTGGAGATGCAGAGGAAA
AGGGCTCCAGCCCCGAAGGTGGAGGAAGTTCCTTTCCCTTGCTCTTCACG
CCTGTCCGCCGGGCAGCAAACCCGATTTCTGTTTTTTGAACCGACCGCCG
GGCAGCGGCGGTGCAGAGGCGGCGGCTGCAGACGGAGCGCGGTGTTGGGG
GCCGAGTCCCCGCGTCCCCTCCTACCCACCCTCGCGCCAGCAGGACCCTC
TGCGTTCCGGTCTTCCGCTGGGACTTATATAGCTGGGCCGCTCCCGGGCT
GGGCCGCGCGAGGTCTCAGAAGACCCGGGCGTGGGACCTGCCGGGGGCGT
GTCCGGGAGCACCGGGTCCCTCCCATTCCCATTCTCGCTCGGTGCCTCGG
GAGCCCTGCTTCGCTCCCCGAAAGCCCTGGGCCGGCCGCTGGTTGTCTGC
GCCCCGCACACCGCACGTCGGAGGGAGGGCGCACCGCGGCGGTCAGCACC
CGCGCCCCCGCACGTGCCTCACCTTCTGTCACCCTTTGGCTGCACACCTT
CGGCTGGGGCTTTCGGAAACCAGCACGATATCAGAGCCTTGGTGAAAGGT
TCTGCCTAGCTGGAGCTCTCCCAGAAGGAAGAGCGGGGCACTCTTCCATT
CCTCCAACTCCCCAGACATCTTGCAGGCACCTTGCGGCCTTCTCTGCTTC
CCTTTCCTCTCCTTTAAACTTCTCATCCTCCTTCCTCCGGATTCCTTCTC
TTCCCACTACCAAATTCAGACTTGGAAGAATCCCACACAGGAACCAAGAG
CACTCTGAACTTGTCAAAAAGAGCGTTTCGTTTCATCACTTTTTGAGAGA
AGATGTCAAACGAGACAATAGACACACTAGAAAGCAAATAGTACATTGCT
AAAGCACCGTACAAAATGAGATACCACCTGTTCCTTCTAATTAGATTTCT
TAGAGGGTGCAGTGGAAAGGACAGGGCAGGTTGCTTGGCTCCAGGTGCAG
CTCTCCAGCTTACCAACTCTGTGAACCTGGGAAAGTCATCTGTACTCCAG
CTTTTATTTTCAACAGTGTAAAATGTGGTTCCAGAGCTAGCCCAGGTCTG
AAATCTCCCTGCTAACTTACCTATAATGATGTAATATGTGTTGGTTGTGT
GGCCTTTCCTAGTTCGATCTTTGTGCAAATTTTATTTTCAATGTTTATTC
TTGTGAAAACTTAAGAAAACAAAAAACTTTTAAAAAAAACAGTGGGAAAT
AATAGGAAACTGGAGAAAGAGGGAAATGGACAAGGTCAGGTCCCAGCTTT
CAGAACCTCTCAGTTAAGCCAAAGATGGATTTGATATTACCAGAATCCAC
GTCCAGCTATTTAGGGCCTCTGGTACTCAACCCTGACTTAAGCCCCCCAA
AAGAGTCAGTGGAAGTCAGAGTCATCTCCTCCAGGAGCAGGAGAAATAAG
ACATGAATAAAGAATAATCTCCAAACCCTCTATAAGTAAAGGTTGTGTAC
TGTGCAAGAAGCCAGTCCTAAGGCACTTCCAAGGGAGGTCAGGCTTGCAG
AGCTTCCCTGAAAAAGGCAATGTTGACCTGACGGGTAGCCTCTGAAAAAA
AATAGAAAGGAGAAGGAGCCAGCGAGGTTTCTCCTCTGCAAACACTTGTT
GAAATAAAGATCAACTGAGGCAAATAAGTGGCATGCATACAGGCAAAGAT
GTTGGGGAAAGCAAGTCGCGTATATAGAATGCATTCTGTTGGCCTGACTG
ACTGGGAAATTGGGTGATGGGGAAGAAAGAGAGAGGTAGGGCAAGAAGTT
GGTGAGGGATGAGGCTGTAGAAGCCAAATGAGAAGCAGAGCCTGCAAATC
CATTATGAGTGTAGCTTCATGTTATCACCTGTGTAGTGCTTTACAGACTA
TGAAGTGCTCTCACATAAATGTCAGATTTAATTTGATAGACAATACAGCT
TTATGAATGATTAGATGTGGAGTCAAAGATTACTCTATGTTTAAGAACAT
GGGTTGCAAGCTCAGTGACAGTACCAATGATTGTCATGAGGCAGTTGGTA
AGGGGGGTGATTTTAGGGGTAAGAAGATTAAACCAATTTTTAAAGTCTTA
GAATTATTTGGAGAGAATACTGTTGATATCCAGATAGAGAGCTTAGGAAG
CTGCATTTAACATGCTAAAAGAGGAAGAAATAGAGGAGGTATTTGGTAGT
CATGATATGGATGCAATGAAAGGGGCATGCTTTTCACGGGAAAATGGGGC
CCAGCTGTGTGACCTTGGGCAAGTGATGAATCTGCTCTGTGTTCAGTTTT
CCCATCTGCCAAGGGATAATGGTACTAATACCCTTCCAGTTATAATGTAT
CTGAAGCACTTAGAACAATGTCTGGTTCATAGTTAAGCCCTCAAATAAAT
GCTACCTAAAGTGACAGGAATTCTTAGAGTGCACTGGCACTCAGGAAATA
TGTTTCCTTTTCTTTTTCCCCTAAACAAACTCCCTGCAGCATTCGTCCAC
TTTTTCTGCTCTGGTCTTTCCTGGACCTGATGAGGAACCTCTTAAGAGGA
AATATGTCTTTTCCTGTCCTTTCAACTCTCTTCACAAAGTCTGGCTTTCT
TTGCTTCAAACAGAAGGCTTTGAACTTTGTTGCATACACAAATGATGAGG
GAAGCTTGTGGAAAGACACTCTGGTCTCTAGCCTGAGGATTCAGACTCAG
AGGACCATGGTAGTCCTCGTTTGGATTTCTGACAAGCTCCATAGATGTTT
CTGATATACTTCCCTCTTTGGAGAACTTTAAGTTAAACATTTTTCTGTAG
CCCACCCATATTCTCCAGATGGAGATCCAAACTGGGCACAAAACTTCCAT
GACTGGGTGACCTTTACAAGTATATATCTGCATCTAAAAAATCGATTTTG
CATACTTTTTTCTATGAACAATGAGCTAATGTTTGGGGAAGGGAGAGTTC
AAATATGAATGTTTTCTCGCCAATTTGTTATGAAAACCACTCCTCTGTGT
GTTACACTTTTTGGAACAGAGCGGAGCCCAAGGCTTTTCTGCCCCTCCCA
GGAACGCTCACGGCTTACTTTCTTTCCCTGCGGTGAGGAACTCCTGGACC
GTGTCGTAGAGGCCGATCCTGAGAGAGGCGGAGCTGATTTGCCGCTGAAG
CCCCGCAGGCAGCCCGCTGTAGAGTTTCATCCGCCCTTCTGTTTTTACCA
CAGCGGTGATTGTTCCCAGGACACCTTTATACCTAATAACACTGGACGTC
GGGCATTCACCTTGGACCTGGAAATAAGAAAGGTGCAGAACAGAAGGGAG
CGAAATTGAGTTCACTCTTAAGCCAAGATTTCCCCCTGTGTTCCTATTTT
CCGTTTCTTTTCTCAGCAACATTCCTCTTCCATCCACCTCCCTCTACCGT
GTGTGTGTGTGTGTGTGTGTGTGTGTGTGTGTGTGTGTGTGTGTGTTCGC
TCACGCGCAGATGGAGGGAGGCTGGCTACAATGCACATTTTGAATATCCC
CCTCCCCAGGAACTCTCCAGAACCCCTAGGGCAGTAGGATAGGACAGAAC
CCCCGAAGAATCAAAGAAAGGAAAAAGCACCTGACTAGTAAACTCAGGAG
AGGCCAATATTCTCAGCTCTTAAAATACTAAGTGCACCCTAAATTTTGGG
AGAATTTGGTAGTAGTCATGGTCAACCCATTCTCTGTGGCCCTGGTGGTT
CTTCAGCTAATCCCAGAAAAAGTTCCAGTTTCCCATCCTAAAGCACCAGC
CCTGGGACAAGGCCAGACTGCTTTGGAAGGTCACCTACCCTACCTAGAAA
CAGTGGTTACACCAAAGCAGAGAATAAATGGAATGCACGCAACAGCGTCC
CCTCTGTCCTGAGACCCCTTGTCTTACCCCTCTGCCTAGCTACCTGGAGC
CGGACTTTGGCCGTGTCCAGCGGGAAGGTGATCACGTCCGCCAAGCACGC
CGCTATTCCAGCTGAGAAGAGCTGGACCCCCAGGGTCGGGTGTACGTCCG
AGGCTGTCAGGCCCCCCATCTTCACTCAGAGACTGGAGATGCAGAGGAAA
AGGGCTCCAGCCCCGAAGGTGGAGGAAGTTCCTTTCCCTTGCTCTTCACG
CCTGTCCGCCGGGCAGCAAACCCGATTTCTGTTTTTTGAACCGACCGCCG
GGCAGCGGCGGTGCAGAGGCGGCGGCTGCAGACGGAGCGCGGTGTTGGGG
GCCGAGTCCCCGCGTCCCCTCCTACCCACCCTCGCGCCAGCAGGACCCTC
TGCGTTCCGGTCTTCCGCTGGGACTTATATAGCTGGGCCGCTCCCGGGCT
GGGCCGCGCGAGGTCTCAGAAGACCCGGGCGTGGGACCTGCCGGGGGCGT
GTCCGGGAGCACCGGGTCCCTCCCATTCCCATTCTCGCTCGGTGCCTCGG
GAGCCCTGCTTCGCTCCCCGAAAGCCCTGGGCCGGCCGCTGGTTGTCTGC
GCCCCGCACACCGCACGTCGGAGGGAGGGCGCACCGCGGCGGTCAGCACC
CGCGCCCCCGCACGTGCCTCACCTTCTGTCACCCTTTGGCTGCACACCTT
CGGCTGGGGCTTTCGGAAACCAGCACGATATCAGAGCCTTGGTGAAAGGT
TCTGCCTAGCTGGAGCTCTCCCAGAAGGAAGAGCGGGGCACTCTTCCATT
CCTCCAACTCCCCAGACATCTTGCAGGCACCTTGCGGCCTTCTCTGCTTC
CCTTTCCTCTCCTTTAAACTTCTCATCCTCCTTCCTCCGGATTCCTTCTC
TTCCCACTACCAAATTCAGACTTGGAAGAATCCCACACAGGAACCAAGAG
CACTCTGAACTTGTCAAAAAGAGCGTTTCGTTTCATCACTTTTTGAGAGA
AGATGTCAAACGAGACAATAGACACACTAGAAAGCAAATAGTACATTGCT
AAAGCACCGTACAAAATGAGATACCACCTGTTCCTTCTAATTAGATTTCT
TAGAGGGTGCAGTGGAAAGGACAGGGCAGGTTGCTTGGCTCCAGGTGCAG
CTCTCCAGCTTACCAACTCTGTGAACCTGGGAAAGTCATCTGTACTCCAG
CTTTTATTTTCAACAGTGTAAAATGTGGTTCCAGAGCTAGCCCAGGTCTG
AAATCTCCCTGCTAACTTACCTATAATGATGTAATATGTGTTGGTTGTGT
GGCCTTTCCTAGTTCGATCTTTGTGCAAATTTTATTTTCAATGTTTATTC
TTGTGAAAACTTAAGAAAACAAAAAACTTTTAAAAAAAACAGTGGGAAAT
AATAGGAAACTGGAGAAAGAGGGAAATGGACAAGGTCAGGTCCCAGCTTT
CAGAACCTCTCAGTTAAGCCAAAGATGGATTTGATATTACCAGAATCCAC
GTCCAGCTATTTAGGGCCTCTGGTACTCAACCCTGACTTAAGCCCCCCAA
AAGAGTCAGTGGAAGTCAGAGTCATCTCCTCCAGGAGCAGGAGAAATAAG
ACATGAATAAAGAATAATCTCCAAACCCTCTATAAGTAAAGGTTGTGTAC
TGTGCAAGAAGCCAGTCCTAAGGCACTTCCAAGGGAGGTCAGGCTTGCAG
AGCTTCCCTGAAAAAGGCAATGTTGACCTGACGGGTAGCCTCTGAAAAAA
AATAGAAAGGAGAAGGAGCCAGCGAGGTTTCTCCTCTGCAAACACTTGTT
GAAATAAAGATCAACTGAGGCAAATAAGTGGCATGCATACAGGCAAAGAT
GTTGGGGAAAGCAAGTCGCGTATATAGAATGCATTCTGTTGGCCTGACTG
ACTGGGAAATTGGGTGATGGGGAAGAAAGAGAGAGGTAGGGCAAGAAGTT
GGTGAGGGATGAGGCTGTAGAAGCCAAATGAGAAGCAGAGCCTGCAAATC
CATTATGAGTGTAGCTTCATGTTATCACCTGTGTAGTGCTTTACAGACTA
TGAAGTGCTCTCACATAAATGTCAGATTTAATTTGATAGACAATACAGCT
TTATGAATGATTAGATGTGGAGTCAAAGATTACTCTATGTTTAAGAACAT
GGGTTGCAAGCTCAGTGACAGTACCAATGATTGTCATGAGGCAGTTGGTA
AGGGGGGTGATTTTAGGGGTAAGAAGATTAAACCAATTTTTAAAGTCTTA
GAATTATTTGGAGAGAATACTGTTGATATCCAGATAGAGAGCTTAGGAAG
CTGCATTTAACATGCTAAAAGAGGAAGAAATAGAGGAGGTATTTGGTAGT
CATGATATGGATGCAATGAAAGGGGCATGCTTTTCACGGGAAAATGGGGC
CCAGCTGTGTGACCTTGGGCAAGTGATGAATCTGCTCTGTGTTCAGTTTT
CCCATCTGCCAAGGGATAATGGTACTAATACCCTTCCAGTTATAATGTAT
CTGAAGCACTTAGAACAATGTCTGGTTCATAGTTAAGCCCTCAAATAAAT
GCTACCTAAAGTGACAGGAATTCTTAGAGTGCACTGGCACTCAGGAAATA
TGTTTCCTTTTCTTTTTCCCCTAAACAAACTCCCTGCAGCATTCGTCCAC
TTTTTCTGCTCTGGTCTTTCCTGGACCTGATGAGGAACCTCTTAAGAGGA
AATATGTCTTTTCCTGTCCTTTCAACTCTCTTCACAAAGTCTGGCTTTCT
TTGCTTCAAACAGAAGGCTTTGAACTTTGTTGCATACACAAATGATGAGG
GAAGCTTGTGGAAAGACACTCTGGTCTCTAGCCTGAGGATTCAGACTCAG
AGGACCATGGTAGTCCTCGTTTGGATTTCTGACAAGCTCCATAGATGTTT
CTGATATACTTCCCTCTTTGGAGAACTTTAAGTTAAACATTTTTCTGTAG
CCCACCCATATTCTCCAGATGGAGATCCAAACTGGGCACAAAACTTCCAT
GACTGGGTGACCTTTACAAGTATATATCTGCATCTAAAAAATCGATTTTG
CATACTTTTTTCTATGAACAATGAGCTAATGTTTGGGGAAGGGAGAGTTC
AAATATGAATGTTTTCTCGCCAATTTGTTATGAAAACCACTCCTCTGTGT
GTTACACTTTTTGGAACAGAGCGGAGCCCAAGGCTTTTCTGCCCCTCCCA
GGAACGCTCACGGCTTACTTTCTTTCCCTGCGGTGAGGAACTCCTGGACC
GTGTCGTAGAGGCCGATCCTGAGAGAGGCGGAGCTGATTTGCCGCTGAAG
CCCCGCAGGCAGCCCGCTGTAGAGTTTCATCCGCCCTTCTGTTTTTACCA
CAGCGGTGATTGTTCCCAGGACACCTTTATACCTAATAACACTGGACGTC
GGGCATTCACCTTGGACCTGGAAATAAGAAAGGTGCAGAACAGAAGGGAG
CGAAATTGAGTTCACTCTTAAGCCAAGATTTCCCCCTGTGTTCCTATTTT
CCGTTTCTTTTCTCAGCAACATTCCTCTTCCATCCACCTCCCTCTACCGT
GTGTGTGTGTGTGTGTGTGTGTGTGTGTGTGTGTGTGTGTGTGTGTTCGC
TCACGCGCAGATGGAGGGAGGCTGGCTACAATGCACATTTTGAATATCCC
CCTCCCCAGGAACTCTCCAGAACCCCTAGGGCAGTAGGATAGGACAGAAC
CCCCGAAGAATCAAAGAAAGGAAAAAGCACCTGACTAGTAAACTCAGGAG
AGGCCAATATTCTCAGCTCTTAAAATACTAAGTGCACCCTAAATTTTGGG
AGAATTTGGTAGTAGTCATGGTCAACCCATTCTCTGTGGCCCTGGTGGTT
CTTCAGCTAATCCCAGAAAAAGTTCCAGTTTCCCATCCTAAAGCACCAGC
CCTGGGACAAGGCCAGACTGCTTTGGAAGGTCACCTACCCTACCTAGAAA
CAGTGGTTACACCAAAGCAGAGAATAAATGGAATGCACGCAACAGCGTCC
CCTCTGTCCTGAGACCCCTTGTCTTACCCCTCTGCCTAGCTACCTGGAGC
CGGACTTTGGCCGTGTCCAGCGGGAAGGTGATCACGTCCGCCAAGCACGC
CGCTATTCCAGCTGAGAAGAGCTGGACCCCCAGGGTCGGGTGTACGTCCG
AGGCTGTCAGGCCCCCCATCTTCACTCAGAGACTGGAGATGCAGAGGAAA
AGGGCTCCAGCCCCGAAGGTGGAGGAAGTTCCTTTCCCTTGCTCTTCACG
CCTGTCCGCCGGGCAGCAAACCCGATTTCTGTTTTTTGAACCGACCGCCG
GGCAGCGGCGGTGCAGAGGCGGCGGCTGCAGACGGAGCGCGGTGTTGGGG
GCCGAGTCCCCGCGTCCCCTCCTACCCACCCTCGCGCCAGCAGGACCCTC
TGCGTTCCGGTCTTCCGCTGGGACTTATATAGCTGGGCCGCTCCCGGGCT
GGGCCGCGCGAGGTCTCAGAAGACCCGGGCGTGGGACCTGCCGGGGGCGT
GTCCGGGAGCACCGGGTCCCTCCCATTCCCATTCTCGCTCGGTGCCTCGG
GAGCCCTGCTTCGCTCCCCGAAAGCCCTGGGCCGGCCGCTGGTTGTCTGC
GCCCCGCACACCGCACGTCGGAGGGAGGGCGCACCGCGGCGGTCAGCACC
CGCGCCCCCGCACGTGCCTCACCTTCTGTCACCCTTTGGCTGCACACCTT
CGGCTGGGGCTTTCGGAAACCAGCACGATATCAGAGCCTTGGTGAAAGGT
TCTGCCTAGCTGGAGCTCTCCCAGAAGGAAGAGCGGGGCACTCTTCCATT
CCTCCAACTCCCCAGACATCTTGCAGGCACCTTGCGGCCTTCTCTGCTTC
CCTTTCCTCTCCTTTAAACTTCTCATCCTCCTTCCTCCGGATTCCTTCTC
TTCCCACTACCAAATTCAGACTTGGAAGAATCCCACACAGGAACCAAGAG
CACTCTGAACTTGTCAAAAAGAGCGTTTCGTTTCATCACTTTTTGAGAGA
AGATGTCAAACGAGACAATAGACACACTAGAAAGCAAATAGTACATTGCT
AAAGCACCGTACAAAATGAGATACCACCTGTTCCTTCTAATTAGATTTCT
TAGAGGGTGCAGTGGAAAGGACAGGGCAGGTTGCTTGGCTCCAGGTGCAG
CTCTCCAGCTTACCAACTCTGTGAACCTGGGAAAGTCATCTGTACTCCAG
CTTTTATTTTCAACAGTGTAAAATGTGGTTCCAGAGCTAGCCCAGGTCTG
AAATCTCCCTGCTAACTTACCTATAATGATGTAATATGTGTTGGTTGTGT
GGCCTTTCCTAGTTCGATCTTTGTGCAAATTTTATTTTCAATGTTTATTC
TTGTGAAAACTTAAGAAAACAAAAAACTTTTAAAAAAAACAGTGGGAAAT
AATAGGAAACTGGAGAAAGAGGGAAATGGACAAGGTCAGGTCCCAGCTTT
CAGAACCTCTCAGTTAAGCCAAAGATGGATTTGATATTACCAGAATCCAC
GTCCAGCTATTTAGGGCCTCTGGTACTCAACCCTGACTTAAGCCCCCCAA
AAGAGTCAGTGGAAGTCAGAGTCATCTCCTCCAGGAGCAGGAGAAATAAG
ACATGAATAAAGAATAATCTCCAAACCCTCTATAAGTAAAGGTTGTGTAC
TGTGCAAGAAGCCAGTCCTAAGGCACTTCCAAGGGAGGTCAGGCTTGCAG
AGCTTCCCTGAAAAAGGCAATGTTGACCTGACGGGTAGCCTCTGAAAAAA
AATAGAAAGGAGAAGGAGCCAGCGAGGTTTCTCCTCTGCAAACACTTGTT
GAAATAAAGATCAACTGAGGCAAATAAGTGGCATGCATACAGGCAAAGAT
GTTGGGGAAAGCAAGTCGCGTATATAGAATGCATTCTGTTGGCCTGACTG
ACTGGGAAATTGGGTGATGGGGAAGAAAGAGAGAGGTAGGGCAAGAAGTT
GGTGAGGGATGAGGCTGTAGAAGCCAAATGAGAAGCAGAGCCTGCAAATC
CATTATGAGTGTAGCTTCATGTTATCACCTGTGTAGTGCTTTACAGACTA
TGAAGTGCTCTCACATAAATGTCAGATTTAATTTGATAGACAATACAGCT
TTATGAATGATTAGATGTGGAGTCAAAGATTACTCTATGTTTAAGAACAT
GGGTTGCAAGCTCAGTGACAGTACCAATGATTGTCATGAGGCAGTTGGTA
AGGGGGGTGATTTTAGGGGTAAGAAGATTAAACCAATTTTTAAAGTCTTA
GAATTATTTGGAGAGAATACTGTTGATATCCAGATAGAGAGCTTAGGAAG
CTGCATTTAACATGCTAAAAGAGGAAGAAATAGAGGAGGTATTTGGTAGT
CATGATATGGATGCAATGAAAGGGGCATGCTTTTCACGGGAAAATGGGGC
CCAGCTGTGTGACCTTGGGCAAGTGATGAATCTGCTCTGTGTTCAGTTTT
CCCATCTGCCAAGGGATAATGGTACTAATACCCTTCCAGTTATAATGTAT
CTGAAGCACTTAGAACAATGTCTGGTTCATAGTTAAGCCCTCAAATAAAT
GCTACCTAAAGTGACAGGAATTCTTAGAGTGCACTGGCACTCAGGAAATA
TGTTTCCTTTTCTTTTTCCCCTAAACAAACTCCCTGCAGCATTCGTCCAC
TTTTTCTGCTCTGGTCTTTCCTGGACCTGATGAGGAACCTCTTAAGAGGA
AATATGTCTTTTCCTGTCCTTTCAACTCTCTTCACAAAGTCTGGCTTTCT
TTGCTTCAAACAGAAGGCTTTGAACTTTGTTGCATACACAAATGATGAGG
GAAGCTTGTGGAAAGACACTCTGGTCTCTAGCCTGAGGATTCAGACTCAG
AGGACCATGGTAGTCCTCGTTTGGATTTCTGACAAGCTCCATAGATGTTT
CTGATATACTTCCCTCTTTGGAGAACTTTAAGTTAAACATTTTTCTGTAG
CCCACCCATATTCTCCAGATGGAGATCCAAACTGGGCACAAAACTTCCAT
GACTGGGTGACCTTTACAAGTATATATCTGCATCTAAAAAATCGATTTTG
CATACTTTTTTCTATGAACAATGAGCTAATGTTTGGGGAAGGGAGAGTTC
AAATATGAATGTTTTCTCGCCAATTTGTTATGAAAACCACTCCTCTGTGT
GTTACACTTTTTGGAACAGAGCGGAGCCCAAGGCTTTTCTGCCCCTCCCA
GGAACGCTCACGGCTTACTTTCTTTCCCTGCGGTGAGGAACTCCTGGACC
GTGTCGTAGAGGCCGATCCTGAGAGAGGCGGAGCTGATTTGCCGCTGAAG
CCCCGCAGGCAGCCCGCTGTAGAGTTTCATCCGCCCTTCTGTTTTTACCA
CAGCGGTGATTGTTCCCAGGACACCTTTATACCTAATAACACTGGACGTC
GGGCATTCACCTTGGACCTGGAAATAAGAAAGGTGCAGAACAGAAGGGAG
CGAAATTGAGTTCACTCTTAAGCCAAGATTTCCCCCTGTGTTCCTATTTT
CCGTTTCTTTTCTCAGCAACATTCCTCTTCCATCCACCTCCCTCTACCGT
GTGTGTGTGTGTGTGTGTGTGTGTGTGTGTGTGTGTGTGTGTGTGTTCGC
TCACGCGCAGATGGAGGGAGGCTGGCTACAATGCACATTTTGAATATCCC
CCTCCCCAGGAACTCTCCAGAACCCCTAGGGCAGTAGGATAGGACAGAAC
CCCCGAAGAATCAAAGAAAGGAAAAAGCACCTGACTAGTAAACTCAGGAG
AGGCCAATATTCTCAGCTCTTAAAATACTAAGTGCACCCTAAATTTTGGG
AGAATTTGGTAGTAGTCATGGTCAACCCATTCTCTGTGGCCCTGGTGGTT
CTTCAGCTAATCCCAGAAAAAGTTCCAGTTTCCCATCCTAAAGCACCAGC
CCTGGGACAAGGCCAGACTGCTTTGGAAGGTCACCTACCCTACCTAGAAA
CAGTGGTTACACCAAAGCAGAGAATAAATGGAATGCACGCAACAGCGTCC
CCTCTGTCCTGAGACCCCTTGTCTTACCCCTCTGCCTAGCTACCTGGAGC
CGGACTTTGGCCGTGTCCAGCGGGAAGGTGATCACGTCCGCCAAGCACGC
CGCTATTCCAGCTGAGAAGAGCTGGACCCCCAGGGTCGGGTGTACGTCCG
AGGCTGTCAGGCCCCCCATCTTCACTCAGAGACTGGAGATGCAGAGGAAA
AGGGCTCCAGCCCCGAAGGTGGAGGAAGTTCCTTTCCCTTGCTCTTCACG
CCTGTCCGCCGGGCAGCAAACCCGATTTCTGTTTTTTGAACCGACCGCCG
GGCAGCGGCGGTGCAGAGGCGGCGGCTGCAGACGGAGCGCGGTGTTGGGG
GCCGAGTCCCCGCGTCCCCTCCTACCCACCCTCGCGCCAGCAGGACCCTC
TGCGTTCCGGTCTTCCGCTGGGACTTATATAGCTGGGCCGCTCCCGGGCT
GGGCCGCGCGAGGTCTCAGAAGACCCGGGCGTGGGACCTGCCGGGGGCGT
GTCCGGGAGCACCGGGTCCCTCCCATTCCCATTCTCGCTCGGTGCCTCGG
GAGCCCTGCTTCGCTCCCCGAAAGCCCTGGGCCGGCCGCTGGTTGTCTGC
GCCCCGCACACCGCACGTCGGAGGGAGGGCGCACCGCGGCGGTCAGCACC
CGCGCCCCCGCACGTGCCTCACCTTCTGTCACCCTTTGGCTGCACACCTT
CGGCTGGGGCTTTCGGAAACCAGCACGATATCAGAGCCTTGGTGAAAGGT
TCTGCCTAGCTGGAGCTCTCCCAGAAGGAAGAGCGGGGCACTCTTCCATT
CCTCCAACTCCCCAGACATCTTGCAGGCACCTTGCGGCCTTCTCTGCTTC
CCTTTCCTCTCCTTTAAACTTCTCATCCTCCTTCCTCCGGATTCCTTCTC
TTCCCACTACCAAATTCAGACTTGGAAGAATCCCACACAGGAACCAAGAG
CACTCTGAACTTGTCAAAAAGAGCGTTTCGTTTCATCACTTTTTGAGAGA
AGATGTCAAACGAGACAATAGACACACTAGAAAGCAAATAGTACATTGCT
AAAGCACCGTACAAAATGAGATACCACCTGTTCCTTCTAATTAGATTTCT
TAGAGGGTGCAGTGGAAAGGACAGGGCAGGTTGCTTGGCTCCAGGTGCAG
CTCTCCAGCTTACCAACTCTGTGAACCTGGGAAAGTCATCTGTACTCCAG
CTTTTATTTTCAACAGTGTAAAATGTGGTTCCAGAGCTAGCCCAGGTCTG
AAATCTCCCTGCTAACTTACCTATAATGATGTAATATGTGTTGGTTGTGT
GGCCTTTCCTAGTTCGATCTTTGTGCAAATTTTATTTTCAATGTTTATTC
TTGTGAAAACTTAAGAAAACAAAAAACTTTTAAAAAAAACAGTGGGAAAT
AATAGGAAACTGGAGAAAGAGGGAAATGGACAAGGTCAGGTCCCAGCTTT
CAGAACCTCTCAGTTAAGCCAAAGATGGATTTGATATTACCAGAATCCAC
GTCCAGCTATTTAGGGCCTCTGGTACTCAACCCTGACTTAAGCCCCCCAA
AAGAGTCAGTGGAAGTCAGAGTCATCTCCTCCAGGAGCAGGAGAAATAAG
ACATGAATAAAGAATAATCTCCAAACCCTCTATAAGTAAAGGTTGTGTAC
TGTGCAAGAAGCCAGTCCTAAGGCACTTCCAAGGGAGGTCAGGCTTGCAG
AGCTTCCCTGAAAAAGGCAATGTTGACCTGACGGGTAGCCTCTGAAAAAA
AATAGAAAGGAGAAGGAGCCAGCGAGGTTTCTCCTCTGCAAACACTTGTT
GAAATAAAGATCAACTGAGGCAAATAAGTGGCATGCATACAGGCAAAGAT
GTTGGGGAAAGCAAGTCGCGTATATAGAATGCATTCTGTTGGCCTGACTG
ACTGGGAAATTGGGTGATGGGGAAGAAAGAGAGAGGTAGGGCAAGAAGTT
GGTGAGGGATGAGGCTGTAGAAGCCAAATGAGAAGCAGAGCCTGCAAATC
CATTATGAGTGTAGCTTCATGTTATCACCTGTGTAGTGCTTTACAGACTA
TGAAGTGCTCTCACATAAATGTCAGATTTAATTTGATAGACAATACAGCT
TTATGAATGATTAGATGTGGAGTCAAAGATTACTCTATGTTTAAGAACAT
GGGTTGCAAGCTCAGTGACAGTACCAATGATTGTCATGAGGCAGTTGGTA
AGGGGGGTGATTTTAGGGGTAAGAAGATTAAACCAATTTTTAAAGTCTTA
GAATTATTTGGAGAGAATACTGTTGATATCCAGATAGAGAGCTTAGGAAG
CTGCATTTAACATGCTAAAAGAGGAAGAAATAGAGGAGGTATTTGGTAGT
CATGATATGGATGCAATGAAAGGGGCATGCTTTTCACGGGAAAATGGGGC
CCAGCTGTGTGACCTTGGGCAAGTGATGAATCTGCTCTGTGTTCAGTTTT
CCCATCTGCCAAGGGATAATGGTACTAATACCCTTCCAGTTATAATGTAT
CTGAAGCACTTAGAACAATGTCTGGTTCATAGTTAAGCCCTCAAATAAAT
GCTACCTAAAGTGACAGGAATTCTTAGAGTGCACTGGCACTCAGGAAATA
TGTTTCCTTTTCTTTTTCCCCTAAACAAACTCCCTGCAGCATTCGTCCAC
TTTTTCTGCTCTGGTCTTTCCTGGACCTGATGAGGAACCTCTTAAGAGGA
AATATGTCTTTTCCTGTCCTTTCAACTCTCTTCACAAAGTCTGGCTTTCT
TTGCTTCAAACAGAAGGCTTTGAACTTTGTTGCATACACAAATGATGAGG
GAAGCTTGTGGAAAGACACTCTGGTCTCTAGCCTGAGGATTCAGACTCAG
AGGACCATGGTAGTCCTCGTTTGGATTTCTGACAAGCTCCATAGATGTTT
CTGATATACTTCCCTCTTTGGAGAACTTTAAGTTAAACATTTTTCTGTAG
CCCACCCATATTCTCCAGATGGAGATCCAAACTGGGCACAAAACTTCCAT
GACTGGGTGACCTTTACAAGTATATATCTGCATCTAAAAAATCGATTTTG
CATACTTTTTTCTATGAACAATGAGCTAATGTTTGGGGAAGGGAGAGTTC
AAATATGAATGTTTTCTCGCCAATTTGTTATGAAAACCACTCCTCTGTGT
GTTACACTTTTTGGAACAGAGCGGAGCCCAAGGCTTTTCTGCCCCTCCCA
GGAACGCTCACGGCTTACTTTCTTTCCCTGCGGTGAGGAACTCCTGGACC
GTGTCGTAGAGGCCGATCCTGAGAGAGGCGGAGCTGATTTGCCGCTGAAG
CCCCGCAGGCAGCCCGCTGTAGAGTTTCATCCGCCCTTCTGTTTTTACCA
CAGCGGTGATTGTTCCCAGGACACCTTTATACCTAATAACACTGGACGTC
GGGCATTCACCTTGGACCTGGAAATAAGAAAGGTGCAGAACAGAAGGGAG
CGAAATTGAGTTCACTCTTAAGCCAAGATTTCCCCCTGTGTTCCTATTTT
CCGTTTCTTTTCTCAGCAACATTCCTCTTCCATCCACCTCCCTCTACCGT
GTGTGTGTGTGTGTGTGTGTGTGTGTGTGTGTGTGTGTGTGTGTGTTCGC
TCACGCGCAGATGGAGGGAGGCTGGCTACAATGCACATTTTGAATATCCC
CCTCCCCAGGAACTCTCCAGAACCCCTAGGGCAGTAGGATAGGACAGAAC
CCCCGAAGAATCAAAGAAAGGAAAAAGCACCTGACTAGTAAACTCAGGAG
AGGCCAATATTCTCAGCTCTTAAAATACTAAGTGCACCCTAAATTTTGGG
AGAATTTGGTAGTAGTCATGGTCAACCCATTCTCTGTGGCCCTGGTGGTT
CTTCAGCTAATCCCAGAAAAAGTTCCAGTTTCCCATCCTAAAGCACCAGC
CCTGGGACAAGGCCAGACTGCTTTGGAAGGTCACCTACCCTACCTAGAAA
CAGTGGTTACACCAAAGCAGAGAATAAATGGAATGCACGCAACAGCGTCC
CCTCTGTCCTGAGACCCCTTGTCTTACCCCTCTGCCTAGCTACCTGGAGC
CGGACTTTGGCCGTGTCCAGCGGGAAGGTGATCACGTCCGCCAAGCACGC
CGCTATTCCAGCTGAGAAGAGCTGGACCCCCAGGGTCGGGTGTACGTCCG
AGGCTGTCAGGCCCCCCATCTTCACTCAGAGACTGGAGATGCAGAGGAAA
AGGGCTCCAGCCCCGAAGGTGGAGGAAGTTCCTTTCCCTTGCTCTTCACG
CCTGTCCGCCGGGCAGCAAACCCGATTTCTGTTTTTTGAACCGACCGCCG
GGCAGCGGCGGTGCAGAGGCGGCGGCTGCAGACGGAGCGCGGTGTTGGGG
GCCGAGTCCCCGCGTCCCCTCCTACCCACCCTCGCGCCAGCAGGACCCTC
TGCGTTCCGGTCTTCCGCTGGGACTTATATAGCTGGGCCGCTCCCGGGCT
GGGCCGCGCGAGGTCTCAGAAGACCCGGGCGTGGGACCTGCCGGGGGCGT
GTCCGGGAGCACCGGGTCCCTCCCATTCCCATTCTCGCTCGGTGCCTCGG
GAGCCCTGCTTCGCTCCCCGAAAGCCCTGGGCCGGCCGCTGGTTGTCTGC
GCCCCGCACACCGCACGTCGGAGGGAGGGCGCACCGCGGCGGTCAGCACC
CGCGCCCCCGCACGTGCCTCACCTTCTGTCACCCTTTGGCTGCACACCTT
CGGCTGGGGCTTTCGGAAACCAGCACGATATCAGAGCCTTGGTGAAAGGT
TCTGCCTAGCTGGAGCTCTCCCAGAAGGAAGAGCGGGGCACTCTTCCATT
CCTCCAACTCCCCAGACATCTTGCAGGCACCTTGCGGCCTTCTCTGCTTC
CCTTTCCTCTCCTTTAAACTTCTCATCCTCCTTCCTCCGGATTCCTTCTC
TTCCCACTACCAAATTCAGACTTGGAAGAATCCCACACAGGAACCAAGAG
CACTCTGAACTTGTCAAAAAGAGCGTTTCGTTTCATCACTTTTTGAGAGA
AGATGTCAAACGAGACAATAGACACACTAGAAAGCAAATAGTACATTGCT
AAAGCACCGTACAAAATGAGATACCACCTGTTCCTTCTAATTAGATTTCT
TAGAGGGTGCAGTGGAAAGGACAGGGCAGGTTGCTTGGCTCCAGGTGCAG
CTCTCCAGCTTACCAACTCTGTGAACCTGGGAAAGTCATCTGTACTCCAG
CTTTTATTTTCAACAGTGTAAAATGTGGTTCCAGAGCTAGCCCAGGTCTG
AAATCTCCCTGCTAACTTACCTATAATGATGTAATATGTGTTGGTTGTGT
GGCCTTTCCTAGTTCGATCTTTGTGCAAATTTTATTTTCAATGTTTATTC
TTGTGAAAACTTAAGAAAACAAAAAACTTTTAAAAAAAACAGTGGGAAAT
AATAGGAAACTGGAGAAAGAGGGAAATGGACAAGGTCAGGTCCCAGCTTT
CAGAACCTCTCAGTTAAGCCAAAGATGGATTTGATATTACCAGAATCCAC
GTCCAGCTATTTAGGGCCTCTGGTACTCAACCCTGACTTAAGCCCCCCAA
AAGAGTCAGTGGAAGTCAGAGTCATCTCCTCCAGGAGCAGGAGAAATAAG
ACATGAATAAAGAATAATCTCCAAACCCTCTATAAGTAAAGGTTGTGTAC
TGTGCAAGAAGCCAGTCCTAAGGCACTTCCAAGGGAGGTCAGGCTTGCAG
AGCTTCCCTGAAAAAGGCAATGTTGACCTGACGGGTAGCCTCTGAAAAAA
AATAGAAAGGAGAAGGAGCCAGCGAGGTTTCTCCTCTGCAAACACTTGTT
GAAATAAAGATCAACTGAGGCAAATAAGTGGCATGCATACAGGCAAAGAT
GTTGGGGAAAGCAAGTCGCGTATATAGAATGCATTCTGTTGGCCTGACTG
ACTGGGAAATTGGGTGATGGGGAAGAAAGAGAGAGGTAGGGCAAGAAGTT
GGTGAGGGATGAGGCTGTAGAAGCCAAATGAGAAGCAGAGCCTGCAAATC
CATTATGAGTGTAGCTTCATGTTATCACCTGTGTAGTGCTTTACAGACTA
TGAAGTGCTCTCACATAAATGTCAGATTTAATTTGATAGACAATACAGCT
TTATGAATGATTAGATGTGGAGTCAAAGATTACTCTATGTTTAAGAACAT
GGGTTGCAAGCTCAGTGACAGTACCAATGATTGTCATGAGGCAGTTGGTA
AGGGGGGTGATTTTAGGGGTAAGAAGATTAAACCAATTTTTAAAGTCTTA
GAATTATTTGGAGAGAATACTGTTGATATCCAGATAGAGAGCTTAGGAAG
CTGCATTTAACATGCTAAAAGAGGAAGAAATAGAGGAGGTATTTGGTAGT
CATGATATGGATGCAATGAAAGGGGCATGCTTTTCACGGGAAAATGGGGC
T
Seq. ID. No: 79 PPARGC1A promoter 1
TACGTTTTAGGATGACAGCAAACATTAATGATTCAAAAAATGAAAACTG
C
CAACGCTACCTGCTGCTCGTAATATTTTATACAGGCGGAGGTGATTCCCA
GAGCCGCTGCCATGTAGGCAAAGCTTCTAGCAGCTAGCTGCCTATAGTGC
AACAGAGAAGTGACTGGCCAATCTCAGCCGACTGGCGACTCTATCCATGG
CTGGACAAGTAGCCAAGACCTTTCCTCCTTAAGAAAAAAATTTAAAGGCC
ACTATTCTAAATGCTTCTGATGTTTATTTGAAAATGACTGCAGGAATTCC
AATGATTAAAATAAATCTTCCTTATTTCCATATGATCTAGTTCCTCAATC
AACTCCTAACAGCATCAGGAATAATACATGATGGAAAAATGAAACTACAG
CAGCTGAGACCCACAGTGCCAGCCAGACAACCACCCACAGGACACTCCGG
CTACTCAAAGGCAATTAGGACTTTGAAGCCACTTATAACCCTGACTCATA
ATGGCTACAGGGCATATGAATTATCCATATCTATTCCGCCTTACCAAGAA
ATCAGAACAAGATAACAATTACAATCCACAAATACGATCTACCAAGGCAA
GGGAGGGCAAATGGAGCCAACATTTGCAAACCCAGCAAAGAGTTAAGAAT
CCCGGGCATGCCTCGCTAGATAGGAAAAATGCATTCACAGACGGCCTTGG
CCAAATTATTTTCACTTTAGATGTGTATCGCTGCACCACATGGACATCTT
ATTTAATCAAATCACGTGTGGGCTTGATCCTCCCCCTTACCCTACGTGCC
CCCAGCTACCCAGAGGAAACACTCGCAGTCTTTTGCCAGATTCATTGTTA
GCAGCTGGGATCCTCCATGCAATTCAATTCGAGTCCATCTCACCAGAGTC
CACAGAAAGTTTTGCTTGTTTCCCAAGTTGCCCAAACTTTCCTTCCGAGA
GCATTCCATTGTGCTGAATGCAAACACGCCGAGGTCCTCCGTCCCCCCAC
TAGACTCCAGAGATTACGAGGAAACTGCTTGCGTTATTTTCCCCCCTGTA
TGAATTGTAGCCAGCAGAGACTGTGGAATTGATGTATTGCTGTCTATTTT
TACACTAGCAGCGAGCAGCTCCACACACAGTCCTGCATAAACTCTATTCT
CTCTCAGCTTCAGGCAGCTCTCCGTGGTACAGGAAGATTTAACCATTGGC
TTCCCTGCCACCGACGCGAACGGAAAACCTTGCACTGCCAGGCGTTTCTT
TTTATTCGCTGGCCAAATCTTTCAACCACGTATAGGAGTTTTAAAGGAAG
CAGCATCGTTGTTTTGTGTTCTGTGGGAAAAGCAATCTAAAATAGCCCAG
CAGGATTATTTAGGATTTTGTAAGTTCTCTGTCTGATGAACAGAGAGAGA
GGGGGGAAAAATCCGGACTAGAGTCAAAACGGACTATATTTGAACACATC
CATTTTAATATTTTTATCTTCGTCTTATTATAAATAGAAATGGCATTTTT
CCAACCAGGCCACTAAACACCACTGGAAAGACTTCAGCTTCTGATTCATA
TCAATATCTTAGAGCATAAATATTGCATATGAGTAGAAACAGTGCCAAAG
TCACATGGAAAGAGTTTCTAACTGCAACAGATACTGATGACAGCAGCTAA
GCTTTGATTTGCTCAAGCCATCTCAGAGCCACCTTAAAATAGCAAACTTA
TTGGAGTTAAGATAAGATTGAGATTCTTCTAAAAGCTCCTGCCTGGACTA
CTTTCCTGGCTCCTCTCTTTGCCCAAGCCCATCCCCCCTTACAGGAATAA
TAGCATCTGAGGGAAGCGTCAGTTGTGGCTGCAGCGCCGAGCCCTGCCCC
AGCTCACCTCGATGTCACTCCATACAGACTCAGAGTCCTGGTTGCACATG
TCCCACGCCATCCAGCTCCTGAATGACGCCAGTCAAGCTTTTTCAACTCC
AATCCACAGTGACACAGAGCACACACTCATGCAGGCAACCAGCCCCTTAC
TGAGAGTGAACTGAAGGCACCTGTCTTACTACAGTCCCCAGTCACATGAC
AAAGCTATTAAAAAGTAGGCTGGGCTGTCACTCACCCAGCCTCCCTTCCC
CTGTGCAGCTTGCTGCTCTAACTCGTGACGTCACTCAAAGGCAGCCCTCT
GCTTCAGTGAAGTAACGCTTTAAAAAAAAAAAAAAAGAAAGAAAAAGAAA
AGAAAGAAAGAAAGGAAACACTTAGACTTTTGGAGGCTTCAAGCATCATG
CTGTGATTAAAGCCAGCTTTGAATGCCACAGACTCTAAACGGAGCCCTGG
CCATATAATAACCAAAATCCCCTGCAATCTTTTTTTAATTTATTTTCCTT
CCAAAACAAGCAAGTGGCAAAGCTCCCTGTTTCATGACAGAGTAACTATA
TTCTTAGCTAAAACGTGTATTCCCTAAGAACTCTTTAATTCTAAGATCTC
CAAGTGGACTCAAGCTCAGTTTGGGACTAAAGCAAGAGCTTATCACATGA
TGCATTTTTATGAGACATTTTCTCATTGAGGGAGTGTTTGAAAGCGCAAA
CGGGGCTTTTTTTTTTTTTTTTTTTTTTTTTGCTTGTTTAATCAACAAAA
TAGCCTGTATGTGTTAAGGTATAAACAAACTCCTCCACCCAGAATTCGGC
TGCCCCCATGGCAGTAGGGAATACCAGCTCCCGAAGAGTTGCTGCAGTTT
TCTTTGCTCCATATAAGGAGAACAAGCCACAAAACCCCATCCTTTCAGCT
TCCTTCTAATTATTTCCATTTCTCTCATAGGCTCCCAGAAAACAAGTGTT
AGTAAATACAGTCGCTGCTGCTCTGCTCAGATCAGCTTTGATTCCCGGCT
CCTATTTTTCATACATGTAGCGTTTCCTCCCTAACTGCCTTAGGGTGCCT
TTTTGAATAAACATTGAATTCCAACCCTAGTGCCCTGGGTTGTGTAGTTT
GCGTTTTGAAATCAAGAATGTCTGTGAACTGAGGGAAAAATACCAACACA
CTATTAGCAGAAATATTCAATTCCTGAGCTTTACTTTCTATTATTTTTCA
GCTTGTACCTTGAGACAATGAGGGCTAATGCAGGTAGGTGCAACTGTTTT
ATGATGACACTACATATTGAATACATAACAAACTCAAGGTACTGGACACA
GTGGAAAAACACTTCCTGTAACATGACAGTGGTCTGACTTAACCCTTCCA
AGTTCCCAGGAGATGTACACGTGTATGTATCTGTGTGAATAAACATGTTT
TCATAAAATGTTAAACCCAATATACTTTTAGAATCAGCTCAATTCTTTTA
CTTGATAATAATTTACCTGAAAACAATCCCCCAGTTACCTAAATATAATT
TGTAAAGCATTATTCTACTGAGGTTAATTTACACTGTTCTTCATTGTCCA
TCTCCTTAAACAGAAAAACATGGTTATTTGCCCTAACACATATTTTAAAT
ATTAAATTGCTCAAATATTAAAGTGAAAGAAAGAACAAAAGAAACAAAAT
ACCAGCAGACTGCTGCTTCTTTCAGACCATACATATTATAAGAGATTTGT
GTGCTGTGAGAGCTGCAACAAGTTGCTAGTAAATTCCCAAATAATGTAAG
GAAGTTGACAGTTTATCATTACTGGGCATAAAAAGGTGGCCAAGAGCTTT
GACATTTTAAGTTTGCTGAATACAGTTCACCAGCACATTTTCCCTTTTAT
AAACAGCAGAAAATTACTGTAGTTTTAATTTTCTAATGAAAGAACAGGAA
AATACAACCTCGGGAATGCTTTTATACAAGCAATTAGTGGCATTACTCTG
CACTCCCCACAAAGAAACACTAATTTTCTTCCCAATTATATTCTGATTTA
ATGTAAAACTTCAGTAAATTATTAAAATTCAGGACAAAGGTCATTGGCTC
TGCCCTCAAGGATCAACTGTGAAATAATATTGGTCACCTGGCCAAGAAGA
GTCAATGTTTATTAATTATTATCATGTGATTCTTCCATTGTGATATTCAC
T
Seq. ID. No: 80 PPARGC1A promoter 2
AGCAAGGACTTTAAACCTTGGTCATGTCAGGTGAATGTTGGCAAGATTT
C
CAGAATGTCTGCATAGCAGGATTGTACCACTTTCTTGATCAGGTGCCTGA
GAACAGGACAAGACCACAAACCATCAAATCATAAGCTGTCACAGCAACTG
CTTTTGGTCAAGTTTTTTCCTCCTCTTCCCTACCTCCTCCCTTTCTTTCT
CTTAGCAAGCTTCAACCAATTTACAAGTGTTCGCTTCCACAAGGACAAAT
TTGGAAGAGTGAAGAACAACACAATTTATCCAAATGTGGGATGAAATGAA
GGAACCAGGCACCTTGCTAAAGCTAGATATCAAAAATTTGCATAAGTAGT
TTTGCAACCACAGAGGTGCTATTTGGAACAAAATAAATTGCTTGTATTGT
ATGCAAGGGTACAAACCTTGACCAACTTTGCGGTGTCTTCCCTGCTTGTA
CATTTCTTAAGCACATCCAGATATTCCTACTAAGAAGTTCAAGTTAGTAT
CCCTCAATAGGGAGACCTAAAAAGATAATTGTTTGTGGACTTAGAAGTCC
TCAACATCTTTATTTGAGAATTAAGAAACATCAGTCCAAAATATTTGAAA
TATAGAATTAGTTCTCCAGGATGTTCTTGTAATTCTTTTGTTTCTTTTTT
GTTTATTTGCTTAATATTAGGTCATATCCCTAGCTCTTTTTTTGAGATCC
AGCACAGAAGTCAGTGACTGGAATCCTGGCTCTCTTCTGAAACATTTGAA
TTACAGTGTTTTGGAATTTCCTATTCTAACAAAGTTTGTTATCCAAACCT
GTATCTCTTTCTACTTTTCCTGTTGGGCTAAAGTGAAGGTGTTGTCGGTC
TGTCTAGTAAATCCAACCTACGAGATAACATAATCACATACATTCATTTC
TCAGGGACCTTGGCGGCTCTTATCTCCTTGCTTTTATGATCTCTTATGAA
CAGCCCGGAATAGCAATTTGCAGCCTGTTTGTAAAGCCATCTTAGTCTGA
ACCCAGCCTTCTTCTCTTTTATCACCCAGCGATATTCTTCAGCTAAATCC
CATTCGCCACAGCAAAATTTGCTCTGAGATCCATTTTAGAATAATATAAG
TATTCTCTGTGTGTATGGAACATATCAAACACGTAATGGAGGGTAATTAT
CATTTCAATGAACTATTTATTAATAGAAGATGTTTTGTGTGCTTTGCTTA
GATTTATTGCTATGCTGCCCACTGTACATTGTAGCTTGAAGCTATTTAGT
TCACGTTCATCAGCCTCTCAGGCAAGCAGCTCTGCTGTTCACACAGTTTT
GGTTTTTCTATGTGCTTTTTTCAACTTTATTTTACCATTATATCACCTAG
AACATTAAACACAATGCTCAAGTTCCACGTGTTAAATCACAATTGCTTTC
TACTAAAGTTCCAAGCTACCCATTTTTATTAACAGTTACTTTATTAAATC
CTTAATAACACAGATAGCATAGTTAGTTAATTTGTGTGTTGAGCCATGGA
TGTTAATTTGTAAACATAATAACATACTGCAATCATTTGCATAATAGCAT
TTTAATCTTTTTAGTAAAAGCTGTAATTCCCTACAAAGTACACTGCAGTA
GAAACTAAGTGACTATTGGTAATAATACAAAAGGCCTTAAAATTCTGATC
CTATTATAGTGCTGGAGCATACCCAAACTGCTTCCATAATCTCTGAAATG
TTACTGTTCTCTGATTTAGGTGTCTGTGTCAAGAATAAAAATGAAGTATA
ACTGGTAGCAGTAGACTATTTCCTAAATGTAATTTAGTATTCACTAAGCA
CCAGCATTTTGCAAATAGCTCTGAGGCTTTCCTTCAAAGTTCAGAGCTCC
AGCTATGCATATTGTGTGAGGAGGACCCTGGAACTCACTCTCAACTCTGT
GCTGAAGAGAGGGCTCTGATAAATCATGCTGATTTGGCGCAGGGAAGGGC
ATCGGCCACCAGGGATTTGACTACTGAGTCTCCTTCCTCCTCTCTGCCCT
ACCAGCCTGATTGCTCCTCCTTGAACTGACGGTTTCTGAGCAGAAGTTCA
GAAAAGAATGAAAAAGTGCAGTGAAGCTTTAAAAAAGCTAGTTTAAAAAA
ACTCAATGGGGGCTCACTCCCTCTACAGCCCATTAACACCCATTGTAAGA
CTGCTCCCCACCCTAACCCCCAAAAAAAGAAAAGGAGAAAGAAAAAAAAA
AGTAGTTGTTTAAGTCAGGCCAAGTCATTTTATGCAAGATCTGGTAATTC
ATTAATCCATCTGGCTGCAAACCTTGAATGAATAACTTGCAGTAGTAATG
GAGATGGGCAGGTCTACAAAATATTAGTGTTTAGAATCTGAAACTGTGCA
AATCACTCCATCATTTTGGAAAAGAAAAGGCACAAAAGTATCTTCTATCA
TCACTTTGGGACAAATGGCTTAATGGATAAGAAGTCATTTAGCTTCAAAC
AACTCTGCCATTGCCAAGAATAAGAGTGTGTATTTCATTGTGGATCTCTC
TCCCCTTTTCCCTGCTCTCATTCTAGCTACAGTAAAGCATAGGATGCTGC
CTTGCTTTCAAACGTACCTCTGAGCGCACAGTCTTAAAAGTTAAAAGGAG
GCCGGGTGCAGTGGCTCACGCCTGTAATCCCAGCATTTTGGGAGGCCGAG
GCAGGCGAATCACGAGGTCAGGAGATTGAGACCATCCTGGCTAACACGGG
GAAACCCCGTCTCTACTAAAAATACAAAAAAAAAAAAAAAAAAAAAAAAA
AAATTAGTTGGGCGTTGTAGCAGTCGCCTGTAGTCCCAGCTACTAGGGAG
GCTGAGGCAGGAGAATGGCGTGAACCCAGGAGGGGGAGGTTGCAGTGAGC
TGAGATCTTGCCATTGCACTCCAGCCTGGGCGACAGAGCGAGACTCCATC
TCAAAAAAAAAAAAAAAAAAAAAAAAAAAGTTAAAAGGAACCTGAGAGGT
TAAGAAGAAGAAAAAAAAATGGCCCTTACTGAAATTGTTTTACATCACAG
GCAATTCATTTTATAAAACCCACAATAAAATTTTTCATTACTCAAAGAAT
AGGCACATAATTATAGGGACAAATTAACCTTGTTAGATGTGAAGTCTGGG
ATAGGGAATGGTGGAGGAACAATTTGGCTCTCTGCTTTAACAATGTGGTC
TTTAATGTTCAGCTTTTTATCTGCTTTGTGTGTTGATTCCATCCAGAGGT
GAGTCATCTGCTGGGCTGTGCTTCCAGGACCTGGCGTAGAGATAGTGGGG
CATGGACAAATAAACACAGCAAGTGTATAAGGTCTCCACACAGACACATG
GTTGCTATACAAATAGACAGTTTGTCCAATGCAGCACCCTGAACCTTGTG
ATTACTTTCACCTACACTATTATCTTTGAAGTAAATCTAAGAAGCAATGA
GCCACTTTTTCCTGATTCTGCTCCAAGATTTCCTCCTTGTGAATTTGGGT
CTCCACTATTTTGGCCCCAAAGATATCCAGGCAGAGTAACTTTGATGAGC
CAGTTGAGACTCCGGGAGGTTGGAAAAACCTACAATAAGCCCATAAAATG
TCCCAGTTTACTTTCTGGAGTGCCTAGAGTATCTGACATGAAATCATTGG
TTTCTCCTATCCCAAAATACAAAATAGCACTTTCTGAAATCCTTGTGCCT
AATGAAACATACACATTCTCACTTTGCAAACTGCAAATGGCTAAGAACCG
AATCAACGTCCATAAAAAAAAAAAAAAAAAGCAGCCAATTTCATCTCTCC
TAGCCACACCTTCCATTAACAAAAGCCACCAGCAGGAAAGTTAGTGCCAG
TGAAAGATGGTCACGTAACCTGCATCCTGTGAGTTGCTATTTATTAGCAG
GGGTTGATACCAAGAGACAGAGCCCTGTGGGTCAGAACTCCTGAGCCATA
TTTTTCCAGTGGTCAGCATAATGGCAGGGAAGAAATAGGGTGATAAACCA
GATGTTCCCAAGATAAGCCAGATGTTATCAAGCCGAGAAGTCTAAAACCA
CAGTGCTTAAATGAGGTGTGTAGATGAACACACACACACACAAACGAACA
ACACTATTTAGTCTCCCTGCAAGATAAGCCGACAGGAGAGGCTGAAAATA
AAGCAGCACACAGGCTGGGTCAGATTGTTGCTTCCTGATTTATGACTCTC
CAAGATGAACATCTAACTGATTTATACCCAGAATTTGGAATTTTGTGGTA
GACTACAAATGCAAAGATAAGTACAACATAAATTTTTAAAATACTCCATA
AGTCTCCATGAGTTTCAACATTCTTCAATGTTCTTATGTTACAGCCAATT
ATCTCTCCACTTAAATGGCTATTATTTTCTTACTTGAAGTCTATGTTCTC
TGTAAGTGTCATTACCAAGTGTGGCAAAAATCTGGCTGTGGGCCGTAAAT
TTAATTTAAATGACAAACATTTATTGATTGCAAATTGGAAATCCCAGAAA
TCTTATAAAAGTGATTATTGTAAATTAATTCAGTCCTCCCACATAACAAA
AAAGAATTTATTCTTCCAATGATTAAATTAAGGTGAGGTTGTATATACAT
GCATTCTTTTTTTCTTGTCTATTTGTTTTGTTAAAATTCCAGGAGGTTTG
CCATTCATAGAAAAGTCTGTCTGACCACAGGCACAGTCAGAAGCAGGAAT
TCATTACCATGGAAGGCTGCAGGATTTCCTCCAAGGAGACAAGTGAAAAA
AAGCAGACACCACGTAAATTTGTGGATTGGGTTAAATATGTTCTGTCCCT
GAGGCAGTGGAAAGATAGAGGGCCCCTTTGTAAAATGACTCACGAGTCTA
GGAAAGTACTTCAGCTCCTAAAGGTGAGCAGAAACTTAACCAGGAACGAT
ATCCACGAGGTTCTGCTTCTCTTTGGAATATCTTTGAGTTAGCATTCCTT
ACCACCTCTCTCTCTATCCGAACTCAGAGTTATCAGGGCTGATGGAATTT
TCCTTCAAGGTACTTTGAATCTGTTCCCTGAATGCCACCCCCTCTGCTAT
CACCTTGTCCCTTCCCTGTACCTGTATCACATTGGGTTTGGAATGCAGCA
CTGCCTGCTGTCTGATTTTCCGTATTTCCTACTTGCCTACACCTCTTACG
ACTGTCAGCATTATCACTCTTAAATACCACCTCCATTATGTTGATCTGCT
AAAAAATCTTCAATGACATCCTACTTTCTAAACAGACTCTAAACATTTCC
AACTGGGTTCTCATGACCCACCGTAATTTTGTACCTACCTTACATCCAAA
CTTACTCCCACTACTGCCACGTTCCCATTAATATGTATTTTACTTTGAGT
AAACCAAGCTCACCTCCACATCCCGTCTATGAAAACGCTCTGCTCTTCTC
TTACAAGGCTTTGTGAATTTATTCTTGCTGTTTATCTCCTCAGCTAGAAA
TACACATACATACACACACATATATATGCATGCATATATGCATACACGAG
CTCGCAATGTTTGCAAATTGCAAATATATATATAATCCTATGTAGGTACA
TACACATATATACATATACATGAATATACAGTCTTGTATTGTTTTCGAAT
TGCTAAGAACTAAAAACATATGCATATACTTGTATATTTGTATACACAGC
CTTGCATGGTTTCCTAATTGTTTTACATTTGTTCATTTTGCCTCCCAAAC
TAGATTTAAAGTCCTTTAGGACACAGGCCTTCTTTTGTGCTTTGTATATA
ATCTTCACATTACCTAACGCAGTTCATTCTATCAATAAACACATGAAGAT
CAACTCACTCATTATGCAACAAGTATTTGTTGAGTTCTTACTTTCTGCTA
TTTTCTGAGAAAATATCTAGAAAAATGTCAGTGAACAAGACAGGCAGATT
CCCTACTCCTCAAATGCTCATGGTCTGTTGAACTGCCAATATTTGTTATT
TTTAACTTTTTGATAGGTACATAATATATGTCCATTTTTGCAAAATTTAG
AATATAACTGAAAGCCAAATGAAGAAAAGTAAAGTATCTATATTCCCACT
AGAATAACCAAGTAGCCTTGAAAGCGTCCACATTCATTTCCTTGATTGCA
TATAGCAGGATAATTTACACACAAATACAAAACCACCACTCACCCTTTTC
TGCTACAAAGTTCACAGTTTCAGGCTTCCTGAGAGTTCCAGCAATGACCA
AACATGAACGCTGGCTACAGCTGTGAGAGTCCTTCTCCCCCCATCAGCAA
AGGCAGTCCTCAGAACGTATTACAAGTAATGACCTAGAGACGACTGCATT
CTCACTTGCCATCATTCTAACGAACCCCAAGTAGCTTCATTCAGAAGTAA
CAGACGAATGAATGAAAGATGCCCATTCAGTTGTATCATGGAGGCATTAA
AGCAGGTGTTAAAATAAACCAGGGACAAGGTTGCCCACAGTGACCCAGAA
AGGCCAATATGCTATTTAAACTAACCCCATGCCATCCATCTTTACAGACT
GCTGTGCTGCAAAAGTAGTCCTTTATTCTCCTCCTTCAAACAATGCACAA
AGGCAACAGAGGGAAGGAAGTTATGTTGCAATATTCTGAAACATAAATCT
TGCTACCAGCCGGTCCAGGGACGCATCCTGTTTTCCTTACACATGGATCA
GATTCACTGGAGGCTGTTACCTCTGAGCCTTTTGCTTTGTTTTTTAAAGT
GGCTTCTGAATCGTACCTGGTCACCTGCATTTACACATCCAAACACAGGT
TGCGTAAGCAGAGAACATTGTTTGCGTGTCCAGTTACTCAACCAACATGT
GCTATTGCATGTTGCTACCCCTCTGCCACCACCCAGAATTCCCTTTCTCA
AATTGTGACATTCAGCTTCATTTTTTATCATTATTAAGTTTGTGATGTTA
AATAAAAATTAAACTTAGATTCAAACATATGGAAGAAACAAAAAGAAGAG
ATACATAATTTACTCTCAACATTTTAAGACTCTGCCACTCTATTATATAT
CCCTAAATCCCACTTTCTGCTTTGATGAGAATTCAGTCGATGGCCAGTTT
CCAGTGGTGTCATTGACGTGCTACTTGAGTCAGGGAAAAGCATTTAACTA
CTCAGTGCCTGATTTTATTCTGGGAATCCAAAGTAGATAATGCTTGTTAT
TGTCAGATCAGAGACAGCAGAGCCCGTGACTGTTTAACTTACTCAAGTAC
AAGGCCTTCTCAAATGTGATGAGGCAGGGCTTTGCAAATTGGAAGAAGGT
GAGTGCAGGTGAAAAGTCCACTGTGAGTGGCCGGCCCTGGGAAGGCACTT
CCCTGAAATGCTGCTTTGGTAGAAGCAACACAAACCTGTGGCCATGAAAG
AGTTATCTGGTTTCATACGAACCTTCTTCACAGGGAAGACCCAATTTTAG
AATGTCACCTGTGCTGAAAACACGCTTTCCTTTCACATTACAGAAAAGAT
GCCTGAGTTTAACACTAGAGGGCAAAGAGTGGAGGTGTCCTCATGCCATG
CAAATACTCCTGAAGCTGCTGCTGTTGGTAAGGGCATTGCCACATATGAC
TTTACCCTTTGCCTCGATAAAGAATGGGCTTTGTGACATCAGAGAAAACT
GTTATGGGAACTTACCTAAACAGTCACTTTTGCCCTCTGACACTAAGAGG
ATACCTGACATATTAGTCCTGGAGCCCCAGGCATCCCGATATCGTTATAC
GACTTCTCATTCTTTATATCCAGTAGCTTACTATGTCTTCTCTCAGAAAT
CTGACCAATTAATCATTAGTAATTTATAAAGGGGAAGCCTGTGACTTTAT
TTAAAAGCAGGGTTTTTCAACCTCAGTGCTTTTTTCATTGGAGGCTGAAT
CATTTTTTGTGGTGGGAGCTGTCCTGTAGGCTGTTGGATGTTTAGCAGCA
TTCCTTGTCTCTAGTCTCTACTCAATAAATGCTCGTAGCATCCCCTTGTC
CCCAGGGATAACAACCAAACATGCCTCCAGATATTGTCAAATGTCCCGTG
AATTGGGGTGTGGGGCGGGGACCAAAATCGCCTTTGGTTGAGAATCATTG
GTTTAAAATGACCTAATGAGAGACTAAATTAAATGAACTTCTGTTTTAGA
GTGCTGAAAATAATTTTATTAGCATAGAATATGATACAGGGAGACAAAAA
GGGTGGCATGGCATATATTAAGCACCTACATGTAGATAATTAACTAGATG
CTGAACCAAGTCTCCTGAAGGTATAAATGCAAAGTACCTCTCACAAATAC
CACAACACAAAATATAGGGACATAAAAGTTATGGAGAAAGTTGTATTTTT
TTTAAAGTAGCCCACCAGAGAGGCATTGACTCCTATGCCATCTCCCCTAC
CGCTAAGTTATTCTCTAGCCTCTACAAAATGCATGGCATTCACCTGATAT
GAAATAGGTGATTTTTAGATAAGAGGTCTATGGAGATGGACATTTTTTAA
AGCATTGTTTATTTAATGCAGCATGACTTAGAATGGGCCTGTAACTTGAC
AAGTGTACTGAGTGAGGAATGTTCAATTTACCAGTTCTAACTGGCCAAGT
CCTCACATGAACTTTCTAAGACCCGGCTGAGAAGCCTGGGAAAGTAGAAC
TAACCACAACCTTCAGTGTTCCCTCCACCTTGTTCTGTCTCCCCAGTCCA
AACTCTACTACCTTAAGCATTCTTCTAAAACCCACTTAATCTCCAGAGGT
ATTACAAAATCTCAGCCATAGGCAGAACCTACAGCCGTCTCGGCTCACCA
GCTAACTCTACCTCCGTGCCAACCAACACTGAAAGGAAAGATTCTGTTCT
ATTTTTAAATATATTGAGCAAAGAAGCAGCTTGATATGTCTTTGTAAAGG
ATGGCTGTCACCCAAATTCATGGGTTCTCTATTTTTATCAAGGATATAAA
CTTTGAAGTCCGATAAATTTGGGGTTCAAATCCCAGGTCTTTATTGTCAA
CTGTGTGATTTGGGGAAAACAAATAACCTCTCTGAGTTTCAGTGTCTGCA
TATGTAAAACAGGAATGATAACAATAGGACCTAACAATAGGTTGTTTTCA
AGGATAAGTGAGATGACATATATTAAGCATGAGTATACCAAAATGTTGGC
CATTATTGTCATTACTATTATTGTTATCAACATTAATATATTCATCATTC
TCTCATGATTTAACAAACATTTATTCAGCATGTATTATATGCTGGGTTCC
AGCAAAACTCAGTTAATATTTTAAACTTGGCTTTCTTAAGACTCAGATTT
CAACCAAATAACATGGAAGTATAAGAAATGTCTTTGCTGATGAATAAGAA
AAGTCCAGCACATAGTAGGTGCTCAGAAAACATTAGATGATGATGATGAT
GGCACTGGCGGTTGTGATAAAGAAAACTAGCCGATATGTCATCCTTCGTT
TAATTCACTTGCCAGAGTACTCACTAAATGTTTGTTCCTATAAAAAATAA
TGTATTTCAAGTAAATGAGTACTGATCTTGTGGGAAAGAATTCCTTTTCT
CAAAGTAACACTTTAATAGGAATAGATTAACATTCTTTATTATAGGGTAG
GCTTATAATAAGAGTCCGGAGTCACGGAGGTAATTCTTGCTACTGAAAAG
TAAATGAATTTTGACTGATTTATAAGTATACAGTAGATTAGTCAAGTTTA
GCAATTCCCATTATATAAAGAAATTAGAGATATGAACCAAAAATATAATT
AATAATAATAATCAAAAGAGCTCACCTAATTAGTAAAGCAAACAAAGGTA
GACATCTACCCCCTTGGTTAAAAAGAAAGGCCTTCTTATTTATTAATTTA
TATGAGGTACCATTTATTGCTCATTCTTTCTGTGGGAAATAGACAGGAAT
AAACAGGTTTAGACTTTTGTTCTTCTTTGTCACTAATCTTTCACAACCCT
TTAGTATTTCAACACAGTTTAACTCACTCACAGTTGTTGATTCCTAGTCC
CTAGTTGATTTATTTTTTAAATCTTTAAAAAAATAAAAACAAAACATTTG
TTTTCCAAATGCTGTTTTTCCATAAATTAGAAAGTATGTAACTGTGATGA
AAAGTCTTTTGATGATAGCAAAGTAAGAACTCATTATGCATTCAGGTCTA
TTACCATGTTAGTCGGGCCCAAGCCAAACTCAATGAAAAATTAATGTTTC
CAAAGGATGTCCTTACCTCAAATATGTTTGAAGGCTCATTGTTGTACTGA
TTGGATATTATTTCTGATTGGTCACTGCACCACTTGAGTCCACCCAGAAA
GCTGTCTGTATCCAAGTCGTTCACATCTAGTTCAGAAAGATCAAGTTCAG
GAAGATCTGGGCAAAGAGGCTGGTCTTCACCAACCAGAGCAGCACACTGC
AGGAGGCAGAAAAAAAAAATTTAAAAAAGCTTCCATTAACCACAGGAATT
TATTAAACTGCAATAGCATGTGATAGGAATTAAGCCTAGTTAATTCAACT
ACACTACCAAAGCTAGTTTCTGGCACTTTAATCACCAGGAAAAACATCCT
AAAATCTTTACTCACTTGGCTCCTGAAGGATAATTTCCCTGCTTTATTCC
ATCCAAAAGCTACCTCCAAACACATGGTAAATACCAAGCTCCACGAAGCT
TTCTCTGATTCTTCCAGTTAGAAATCAACTAGATATTTTTGGACCTCTTT
GTACAAAATCCTTGATACCTGTTTGATAGTTACTGCTTCCAAAGTTTTAA
ACTGTCTTATAGTTAACCATAATACAAACTTGATTTTACCTTGGAAATTT
TATCACACATAATAGACACTCAATAAACTATTGCTAAATGGAAAAAGAAC
AGAGGAACTACACTAGGAAAAATTTTCTCCATTTACACTCCTTTTAAGCA
TTATAATCCAAATAATTCATTTAGCAACTAATGATGCAAAGCCCTTTGAC
TCATCTGTGGTTCTCCTGAATTGTTATTTAAATCATTTACTCTTATTTGA
CTCTTCATGACTTTCTGGACTTCATCCTTAAAGGCAAAAGCAAGTTCCTT
AAGATGAAGGACAAAAATCATAATTTTTTTTCTATACGGCTTTTGTATGT
AACTTGCAAGATGTGTGTGTGTGTGTGTATGTGTTTGCACACGTATGTGT
GTGTACGGTTGGAATCACCTAGTACCTGGAAAAGAAAACTCACAATAGCA
TACCATCACTATATTTTGTTCATTATTTATGTATATCTAAACTGTGCTCT
ACGTGAATTCAAAATAACTATTTTTGTTTAGAGTTTTAAAATAATTTCTA
CATATTGAGAGGTTTAAAATATCTCTACCATGGCTGGGCTGCTTATCACC
AAGATTATTTTAGATTGTCAAAGCATTTCATCCCCTTACATGAAATAGAC
AGTCTTTTTAACTGTTTTACCATGCGGCTTTTGGAGGCAAGTGGACTCCC
CTTACCAATGAGGTGGTCTCCTAAACTTGAATTGGCTACCTAACCGCTAA
TGTCAGTCCGCAGACAGTAAAGAACACTGCAGGAACTCCTATTGCAGGGG
TCTGGCATTGCCAGAACAGTAAGGACTTCATCCACCTGTAAGTGGGGGTG
TCTAAATCAGGAGGCACACACTGACGCAGCACCCTCATGGCCCTGCCTTC
CAAGGTCAGGAGAGATGCTGTGGAATTGTTCACATCCAGGCTGCAGTTAG
GCATAAAATGAAAGATGCTCCTTGACATTTACATTTTGAATGAAATAAGT
TTTATGCAGATTTTCTTTCTCAAGCACCCCTGTTATCCCTGGCAGGGATT
CCTAGCAGGCCACATTACACAGGTCATATGCAGCAGCGTGATCAAAAGCA
ACACGCTGAGAAGTAAGTACTGGGTAATGGAGTCAGCAGTTGGTAAGGGG
AGGATGGGGGGAGACTGAAAATCATTGCCATCCTTCTCTAAGACCATTGG
ACCCCAGTGAGATCCCTGGCAGCAGGGACAAGGGAATGCAAAGAAAGGGC
CCTCCTCACCATCAAAACTCTTTCCTTATCATTTTATTGGTCTTTCCTTT
TATTATCTGAGTTAGCCCAAAATTACAGATCAGTATACTTGACAAGGCAT
ATAAGTAACTGGAGGCTGGGCTGAAGAGATCTCGTTCTGGTACAATGCTC
ATATTTGGTTGTGGATGTAACACTGCCCAGACTCCATTCTTGCCCACACA
GCTGATCCTCCAAGCCCAAACACACTATCTTAACTTTTCCTCAATGTCAC
CAATTCAGAGATGGGGGAGAACTTTTTCAACGGTGCAAGATACACTAGGA
TTTTATAGGGGCATAGTCTTTAAAAAAAAAAAAAAAAAACATGAACAACA
AAAATAGCAACACAAAAGTAAAAATAATCAAGAAAATGTAGGCTGTCTAC
ATATGGCTTAAGTTCCTGTTGTGAAACTTAGGAACCAACCATGTTTTGGA
ATGCTAAAGTAGCATACTGATTTGTGGACAACTTCTTCCTGTCCCTCTCT
TTAACTGCCCTCTTTGGCATGACTTGCTCATAACCCTTTAACCTTCCATA
AATATGTATTTTTAAATAACTTTTGAAAACCATTGAAAAGACCAGGTCAA
TTTGTTGTATTGGTGCTATCTTGTCATTTACATATTCCTTTCCTTCCTCT
CACTTGTTCGCTCGTGCGCTCTCTCTCTCTCTCTCATTTTGCTTTTATGT
TATGGTTGAGAAAAGTGCATGTGTTCTTGCCAATGTTGACATGCTAATAA
AATCTGTGCTTCTGAATGTGGCATTATCCTGCTAATCTAAAACACATTAG
TGGTAGCAAGTGTTCCTGTTATTAATTTATATTTGGCCTTTGGACAATGC
CAGACTTAACAGTCCAAGCCCTGTAACTGTCCTCATCTGACATGCTGATA
TTTACTGTTTCCCTATACCTCTGTAACAGGTGTGCTTTATTGCAATTACA
CAGACACATTCTACTGCCTCTTTGCAACTATGACAGCTATTTCACAACTG
TAGCCTCAATTTTATAATAAGATTTAAGAGGATAAAGCAGTCACTAAACA
CAAAAGGCTAATTATTGTATGTTATTTCACCCCAACAGACTTTTCTCTTA
GTTAAGGAGCAGACAATAAATTAGCTTAACTGCACTGATGATAATGAATC
ATTATAATTAAGTATAATTCTCCATTAACCTTTTGGAGGTTTTTGTGCCA
TTGATGGTAAATTTTAAAGAGAGAGAGAGAAGAAGGAGTGGACTTGGCCA
CATTAATTATATTAATGAAAAAAGAAACTGGGTTCTTAAAAAAATTAGTT
AAGACAGGGGAAAGGGGGTGGAGAGAGGGGCAGTAAAGAATGACAGTCTT
AGTGGGGTGTATGTCATGTGAATTGTCTCTATCAATCTTAGTAACCTTAC
TGTATATCACCTTCATTAAACTGTGAAACACGGGGCACTCCAAGTAGCTA
TAGTGATATAGGCCTATATTAACCAATGTACTACCCTGTCATTATGTGCA
AAAGGGCATAATACGTTTTAGGATGACAGCAAACATTAATGATTCAAAAA
ATGAAAACTGCCAACGCTACCTGCTGCTCGTAATATTTTATACAGGCGGA
GGTGATTCCCAGAGCCGCTGCCATGTAGGCAAAGCTTCTAGCAGCTAGCT
GCCTATAGTGCAACAGAGAAGTGACTGGCCAATCTCAGCCGACTGGCGAC
TCTATCCATGGCTGGACAAGTAGCCAAGACCTTTCCTCCTTAAGAAAAAA
ATTTAAAGGCCACTATTCTAAATGCTTCTGATGTTTATTTGAAAATGACT
GCAGGAATTCCAATGATTAAAATAAATCTTCCTTATTTCCATATGATCTA
GTTCCTCAATCAACTCCTAACAGCATCAGGAATAATACATGATGGAAAAA
TGAAACTACAGCAGCTGAGACCCACAGTGCCAGCCAGACAACCACCCACA
GGACACTCCGGCTACTCAAAGGCAATTAGGACTTTGAAGCCACTTATAAC
CCTGACTCATAATGGCTACAGGGCATATGAATTATCCATATCTATTCCGC
CTTACCAAGAAATCAGAACAAGATAACAATTACAATCCACAAATACGATC
TACCAAGGCAAGGGAGGGCAAATGGAGCCAACATTTGCAAACCCAGCAAA
GAGTTAAGAATCCCGGGCATGCCTCGCTAGATAGGAAAAATGCATTCACA
GACGGCCTTGGCCAAATTATTTTCACTTTAGATGTGTATCGCTGCACCAC
ATGGACATCTTATTTAATCAAATCACGTGTGGGCTTGATCCTCCCCCTTA
CCCTACGTGCCCCCAGCTACCCAGAGGAAACACTCG
Seq. ID. No: 81 PRDM16 promoter 1
GTTCTCTGCCCCAACCCCTGTAGAAAGATGGTCAGGGTGTGGGGAGGGT
G
GGTAAAGGGGTCGATAGACCCTCGAAAGGACGGCAGGGAATGGGGCTGTG
CCATTGCTCATGGCCCTTCTCTAGGCGACACTCACCTGGTGCCAGAAACA
GCAGAGGTAGCGGCCAGTGAGCGCTTCCTCCATCCGGGGCTCGGGCGGCG
AGCTGGCTGCAAGACTGGGCACCCTGGAGCCGTGGGAGCCTTGGGAGCCG
AATCTGGGTTCGAATCTGAGCAGGGAAGCGGTGCCGACCGCGGAGGATCC
AAGGCAGAAGGCCCAGCGCACCGCCGCCGCCAGCTCCTGCCCAACCCGGC
TCCGCTGGGCTTTTCCTCTGGGGTGGCGCCGGCCCCGCGCCCCTCCCCAT
CCGGGGCGCCTCTGCTAGGACACCGGGGTCTCGGGGTTTCTCAGGGCTGC
TGCGTTTCACCTCCTTTAACGCGGAGGCGCGGAGTTGCACGTGTGGGTCT
CAGTGGAGCCGCCACAGGTCTTATTACACAACAAAGGGCAGGGAGGGCAA
GGCCAGGAGCCTCGCGGGGCGGCGCAGAGACCCCGCATCGCGGGCGCGAC
CCCTGCCTCGGGCTCAGGCTGACTCTGCGGCAGCTCCAAGTCCCGTTGCC
GGGCAGGACTTCGGTGCTCCGGAAGGGTGCGGGGCAGCAACCTGGCCAGG
GCAGCGCCTCGGTTTCCTTCCAATTCATTCTCGATTCCCAGATACTCAGA
TGAGAAATCCGCACTCAGGCCGTCAGGCTCCTGGGCCCGGGCCACCCTGC
CTGCGCTCAGCCCGGCCCCTCCGCCGCAGCGCCTCGGAGTTTGGGGGCTA
CAGGGGAAGAGGAAGCGTCTGGTCGGCTCCCGTCCGAGCTGCTCCACCTG
CGCCGCTGGCCTTTCCGCCCGCTACGCACCTGGGCCCCGAGAGGCCAGAG
GGGAGTGTCCCCAGTGGCCCGGGTCCCCGAAGGTGGCCCAGCTCGGGCGC
TCGCGCAGCCGGGTACTCACCTTGGGGGCGCGGCGGCAGGAGAGCGCGGG
CGGAGAGGTGCGGGCGCCCTGGGACGACCAGCCGAGGACGCCTGGTGTTT
GGACGGCTGCCCCCGCGCTGTCCCCCCGTGCGAGGCGTGTTGGCCTCCGT
GGCCTCGCGAGGGACCGAGGGCGGCGGGTCCGAGTGCCGCCCGCCGGCCT
GGGCAGTGGCGGAGCGCGGAGCTGGCGGCGCCTCCGCTGTTGTTTTTGAG
CCGCGAAGCCCGAAGCAGCAGCCGTTGGGTGGGGGCGAAGGGGAGGAGGC
GGGGGCCGGGAGGCGCGAGGGGGGCGGTGGGGCCCGGAGGGGGCTTCGGA
GGAGCCGGGTTCTGGCGCGCGAGCCCCGACGCCCCCTCCAGCGATCGCAG
GGCGGGGACGGGGGCCGAGGGGAGAATGCAGGAGAAAAGCGGGCCGGGGC
GACGGGGAACGCAAAGCTTTTAAATTAGTAATCAAACGCAGTAAAACGGA
GCAAAACCAGCCCGGACTCGCGTCCGAACTATCGGCGCGTGTGCCCCGAG
ATCCAAGGAAGTCGTGTGGAAAACCGCAGCCGCCGCCCCATCTTCGGCCC
CGGCTGGGGCGGGAGCAGCAGCGCGACGCGCCGCGACGCAGGGGCAGGGT
GGGGGGATAGTCACCTCGCCTCCGGGGCCACCTGGGTCCTTTTAAAGTGT
CCGGGGTGCGGGCCCCGGGCGGGAGCGCCGAACACGGAATGTTTTCTTAA
AGGGCCAGTTCCGAGCTGCGCTGAAAAGGGGGGGGGGGAGAGGGGGGAGA
GATAAGTGAGACGAAGACCGAGAAGGCCGAGGAGAGCCCCCCAATCCCGC
GCCGAGGCGGCGGCGGCGGCGGCGGCGGCGCGACGATGAGGATGATGAAT
ACATTGCAAAGTTTTTTTGGCCCCGGCGAGGGGTGTCAGATTGAGTGCTC
TGTGCGCATGTGCGAAGGTGTCCAAACTGACAATGCTGGGGAGATGAAGA
TAGTGTGTGGCTGCTTCTGGACTCAAGGAGGAGGAGAGAGATTCCGCGAG
CCGACACCATGCGATCCAAGGCGAGGGCGAGGAAGCTAGCCAAAAGTAAG
TCTCCCGCGCTCGGCCGCGCCGCGCCGCCGGGGCCCGGGCCGCCGGGCCG
GGGCGCCCGGGCCAGGGGTGCGCGTCGGGGCGCGGCCGGCGCGCCTGCGG
CTCCGGGCCCCCGGCTCGGCCGCGCGGCCCGGGGGGCTGCTCCGCCTCCC
GCGCTCCGGGGCGACCGGGCTCGGCGCGGAGGCTCGGGGCGCCCGGGCCG
CGCGCTCCCCGAAGGCGCCGGCCCCCTCCCCGCGGAACCCCCTCCCCCCC
CACCAGTGTCAGGCGCTCGGGCCCGGGAACCCGAGGCGCGCGGTGGGGGC
CGGGGAGGGGGGCGGAGGGGGAGGGCCGGGGGTGGAGGGGAGCGAAAAGT
GAAAGTTAGCGAAAAGTTGACGAAAGGGGAGAGCAACTTTTTCCTCCCCG
CTCGCTCTCTCCCTCTCTCTCTCCGAGTGGCTCTCAGTCCTGGTCAAATC
ATATTCCGGGCTTTTGAAATAGTGGGCGCTAGAGCACCGCCTTTGGCGTC
CGCCAGCCGGGCGCAGAGGAGGGAGACCCCGAGCCGGGGAGGGGCGGAGG
AGGGGGCGCGGGCCGGCGCCCACCCGGCTCCGCGGGCGCAGGGGCAGGGG
TGGCGACGGCGGGACAGCCGCAGCCACTTGGGGAGCAAAATGGAGACTTT
TGCCCGGGCTGGGAACTGTGGTCGTGAAGTTTATCCCCGGCTGTGCCCCG
CGTGGTGGAGGGCGCTCCTCTGGGACGCCGGCAGCCTGCGCGGGCCGCCG
GGGCGACAGTGCCTCCGACAGGAGGGACCCACCCGGCCACCTCCCGACCC
GCCGCTGCCGCCCTCGGGCCCGCGTGGACCCCTCTGTACCCGCTTGCCTG
GCACAGCCGGGCCCGGCGGGGACAGAGCTGCCCCGGCCAGGCTGCCCGGT
CCTCGGTCTTACTTTCTCTTTCGCTCCGTCTCGGCCGAGCCCCGGGCTCC
GCGCGCCCCGCCTGCCGCCAGCCGGTCCTTGCCGCGGGCCGGGTGCTCCA
GCCACCCGCGCCCTCGCCCGCCACAGCGCCTCCGAGCAGCCCGCGCGCAC
CCCGCAGCCCCGGTCGCCGGCGCCATCCGCGGTGAGGCGGGCGGGCAGGT
GAGGACAGGCGGAGCCGCGGCCCGGCCAGCGCGGCTCCCGCAGCCCCGCC
GAAGCCCCGGCTGCAGCGAAGCGGCCAGCGCCGCGCGGAGCGGAATCGGC
GCCGCCTGCTCCCGCCGCCCCCTCCTCTGCAGGCCGCCCGCCCGCCCACC
CGCAGAAGAGGCGCCCGGGCCGCTCCCCTCGAGGTCCCCGCCCGACCCAC
GCCGTTCCGTCCGCGCCTCCGGGGCCGGGTCCCTCCAGGCCGGCCTCGCA
GCTCCCGGGGCCGAGCCCCCAAACGTGAGCCGGGTGCGCGCGGGACTCGC
GGCGGTTTTCCGAAGCTCCGCTCCCTCGGCTCGGGGCCCGCCCGGCTCGT
CCCAACAGCTGCCCCGCGGCCGTAGGCCCCGCGCTCTGTGCACCAGGGAA
AAGCAGCTCCCGAGTTTGTGCCGGACGCTCCTGCGCCCCGCCATCGGCCG
TTCCCGGCCCCTGGGTGAGGCGCCCCCTTCCCGCCGCGCTCCGCTGCCGG
GTGCCCTCGGCCTCGGCCTCCCGTGACCTCTGACCCCTCTTGGTGCGAAC
AAGACCGGGCGTTTCGCCGCCGACGCGAAGGGGCTGTCTGTGCGCGGCGT
TGCGGGCCCTCCGCGCGTGGGGTGTGCGTGTGCGTGTTCGGGTTCGGTTC
TGTGTGTGCACCGCGGGCCTGCTCAGAGTCGGGACCACCGGGCTGCGTGT
GGGCCGCCAGGTCAGGCCCTCGCCCAGGATGGGCCCTAAGCTGGAACGAG
AAAGGGGAAACCGGCCGCGCTGACGCCGGGGCCTTCGCTGCAAGCGAGAG
G
Seq. ID. No: 82 PRDM16 promoter 2
TGGAGGAGGAGGCAGGGAGAGGGCCTGAGAGAGTTGGGGCACCCGTAG
AA
GAAGGCCAGGACTGCACAACCTTGGCATCTGTTGGCTACCTCCAGGCCAG
GGGCAAAGGAGGCCTCTGCTGGGCTGCAGGGTGAGGGTGGGGAAGGGACC
CTCTCTGCAGGTGGGAGTGGTTCGCAGGCACCATAGTAACTTCCCCAGGC
CTCCTCCCAAACCTGCAGAGGCTTCAGGCAGGAGCAAGGGGGACCTGGGT
GCAGAGATATGGGAGTGACCTTGAGTCCTTTCTAGCCAGCAGCTCCAGCC
AGGTTGGGCCCACCGTTGGGAACCTGCCCTTGGCGGGGTTTAGGCAAGAC
AGCGCCCGTGTTCCTGGGCTCCAGCACCTCCCACCCACTGGGGCGAGCAG
GTTCAGAACCGGCCTTGGCGGTGCAAAGCTGCAGCGGTGCCCTAGTTGGC
TAGGCAGATCAAGACCTCCAGAGCCGGGCCTGTAAACTGAGCTGTTCTCT
GCCCCAACCCCTGTAGAAAGATGGTCAGGGTGTGGGGAGGGTGGGTAAAG
GGGTCGATAGACCCTCGAAAGGACGGCAGGGAATGGGGCTGTGCCATTGC
TCATGGCCCTTCTCTAGGCGACACTCACCTGGTGCCAGAAACAGCAGAGG
TAGCGGCCAGTGAGCGCTTCCTCCATCCGGGGCTCGGGCGGCGAGCTGGC
TGCAAGACTGGGCACCCTGGAGCCGTGGGAGCCTTGGGAGCCGAATCTGG
GTTCGAATCTGAGCAGGGAAGCGGTGCCGACCGCGGAGGATCCAAGGCAG
AAGGCCCAGCGCACCGCCGCCGCCAGCTCCTGCCCAACCCGGCTCCGCTG
GGCTTTTCCTCTGGGGTGGCGCCGGCCCCGCGCCCCTCCCCATCCGGGGC
GCCTCTGCTAGGACACCGGGGTCTCGGGGTTTCTCAGGGCTGCTGCGTTT
CACCTCCTTTAACGCGGAGGCGCGGAGTTGCACGTGTGGGTCTCAGTGGA
GCCGCCACAGGTCTTATTACACAACAAAGGGCAGGGAGGGCAAGGCCAGG
AGCCTCGCGGGGCGGCGCAGAGACCCCGCATCGCGGGCGCGACCCCTGCC
TCGGGCTCAGGCTGACTCTGCGGCAGCTCCAAGTCCCGTTGCCGGGCAGG
ACTTCGGTGCTCCGGAAGGGTGCGGGGCAGCAACCTGGCCAGGGCAGCGC
CTCGGTTTCCTTCCAATTCATTCTCGATTCCCAGATACTCAGATGAGAAA
TCCGCACTCAGGCCGTCAGGCTCCTGGGCCCGGGCCACCCTGCCTGCGCT
CAGCCCGGCCCCTCCGCCGCAGCGCCTCGGAGTTTGGGGGCTACAGGGGA
AGAGGAAGCGTCTGGTCGGCTCCCGTCCGAGCTGCTCCACCTGCGCCGCT
GGCCTTTCCGCCCGCTACGCACCTGGGCCCCGAGAGGCCAGAGGGGAGTG
TCCCCAGTGGCCCGGGTCCCCGAAGGTGGCCCAGCTCGGGCGCTCGCGCA
GCCGGGTACTCACCTTGGGGGCGCGGCGGCAGGAGAGCGCGGGCGGAGAG
GTGCGGGCGCCCTGGGACGACCAGCCGAGGACGCCTGGTGTTTGGACGGC
TGCCCCCGCGCTGTCCCCCCGTGCGAGGCGTGTTGGCCTCCGTGGCCTCG
CGAGGGACCGAGGGCGGCGGGTCCGAGTGCCGCCCGCCGGCCTGGGCAGT
GGCGGAGCGCGGAGCTGGCGGCGCCTCCGCTGTTGTTTTTGAGCCGCGAA
GCCCGAAGCAGCAGCCGTTGGGTGGGGGCGAAGGGGAGGAGGCGGGGGCC
GGGAGGCGCGAGGGGGGCGGTGGGGCCCGGAGGGGGCTTCGGAGGAGCCG
GGTTCTGGCGCGCGAGCCCCGACGCCCCCTCCAGCGATCGCAGGGCGGGG
ACGGGGGCCGAGGGGAGAATGCAGGAGAAAAGCGGGCCGGGGCGACGGGG
AACGCAAAGCTTTTAAATTAGTAATCAAACGCAGTAAAACGGAGCAAAAC
CAGCCCGGACTCGCGTCCGAACTATCGGCGCGTGTGCCCCGAGATCCAAG
GAAGTCGTGTGGAAAACCGCAGCCGCCGCCCCATCTTCGGCCCCGGCTGG
GGCGGGAGCAGCAGCGCGACGCGCCGCGACGCAGGGGCAGGGTGGGGGGA
TAGTCACCTCGCCTCCGGGGCCACCTGGGTCCTTTTAAAGTGTCCGGGGT
GCGGGCCCCGGGCGGGAGCGCCGAACACGGAATGTTTTCTTAAAGGGCCA
GTTCCGAGCTGCGCTGAAAAGGGGGGGGGGGAGAGGGGGGAGAGATAAGT
GAGACGAAGACCGAGAAGGCCGAGGAGAGCCCCCCAATCCCGCGCCGAGG
CGGCGGCGGCGGCGGCGGCGGCGCGACGATGAGGATGATGAATACATTGC
AAAGTTTTTTTGGCCCCGGCGAGGGGTGTCAGATTGAGTGCTCTGTGCGC
ATGTGCGAAGGTGTCCAAACTGACAATGCTGGGGAGATGAAGATAGTGTG
TGGCTGCTTCTGGACTCAAGGAGGAGGAGAGAGATTCCGCGAGCCGACAC
CATGCGATCCAAGGCGAGGGCGAGGAAGCTAGCCAAAAGTAAGTCTCCCG
CGCTCGGCCGCGCCGCGCCGCCGGGGCCCGGGCCGCCGGGCCGGGGCGCC
CGGGCCAGGGGTGCGCGTCGGGGCGCGGCCGGCGCGCCTGCGGCTCCGGG
CCCCCGGCTCGGCCGCGCGGCCCGGGGGGCTGCTCCGCCTCCCGCGCTCC
GGGGCGACCGGGCTCGGCGCGGAGGCTCGGGGCGCCCGGGCCGCGCGCTC
CCCGAAGGCGCCGGCCCCCTCCCCGCGGAACCCCCTCCCCCCCCACCAGT
GTCAGGCGCTCGGGCCCGGGAACCCGAGGCGCGCGGTGGGGGCCGGGGAG
GGGGGCGGAGGGGGAGGGCCGGGGGTGGAGGGGAGCGAAAAGTGAAAGTT
AGCGAAAAGTTGACGAAAGGGGAGAGCAACTTTTTCCTCCCCGCTCGCTC
TCTCCCTCTCTCTCTCCGAGTGGCTCTCAGTCCTGGTCAAATCATATTCC
GGGCTTTTGAAATAGTGGGCGCTAGAGCACCGCCTTTGGCGTCCGCCAGC
CGGGCGCAGAGGAGGGAGACCCCGAGCCGGGGAGGGGCGGAGGAGGGGGC
GCGGGCCGGCGCCCACCCGGCTCCGCGGGCGCAGGGGCAGGGGTGGCGAC
GGCGGGACAGCCGCAGCCACTTGGGGAGCAAAATGGAGACTTTTGCCCGG
GCTGGGAACTGTGGTCGTGAAGTTTATCCCCGGCTGTGCCCCGCGTGGTG
GAGGGCGCTCCTCTGGGACGCCGGCAGCCTGCGCGGGCCGCCGGGGCGAC
AGTGCCTCCGACAGGAGGGACCCACCCGGCCACCTCCCGACCCGCCGCTG
CCGCCCTCGGGCCCGCGTGGACCCCTCTGTACCCGCTTGCCTGGCACAGC
CGGGCCCGGCGGGGACAGAGCTGCCCCGGCCAGGCTGCCCGGTCCTCGGT
CTTACTTTCTCTTTCGCTCCGTCTCGGCCGAGCCCCGGGCTCCGCGCGCC
CCGCCTGCCGCCAGCCGGTCCTTGCCGCGGGCCGGGTGCTCCAGCCACCC
GCGCCCTCGCCCGCCACAGCGCCTCCGAGCAGCCCGCGCGCACCCCGCAG
CCCCGGTCGCCGGCGCCATCCGCGGTGAGGCGGGCGGGCAGGTGAGGACA
GGCGGAGCCGCGGCCCGGCCAGCGCGGCTCCCGCAGCCCCGCCGAAGCCC
CGGCTGCAGCGAAGCGGCCAGCGCCGCGCGGAGCGGAATCGGCGCCGCCT
GCTCCCGCCGCCCCCTCCTCTGCAGGCCGCCCGCCCGCCCACCCGCAGAA
GAGGCGCCCGGGCCGCTCCCCTCGAGGTCCCCGCCCGACCCACGCCGTTC
CGTCCGCGCCTCCGGGGCCGGGTCCCTCCAGGCCGGCCTCGCAGCTCCCG
GGGCCGAGCCCCCAAACGTGAGCCGGGTGCGCGCGGGACTCGCGGCGGTT
T
Seq. ID. No: 83 FFAR1 promoter
CACTCCAGCCTGGGTGACATACTGAGACTCTGCCTCAAAAAAAAAAAAA
A
AAAAAAAAAAAGGGTAGGCATGAGGCAGACTGTGAAGCTGAGTGGGGAAA
CAAGGTGAAGGAAGGGGATAAAATGTCCTCTGAGGAGACCTGGGCTGTCA
GAGGCCAGGGAAGGGGACGAGGGTTGGGGAACAGGTGGTTAGCACTTCAT
CCTCGTCTCCCTCCCAGGTTAGAAGGGCCCCCCTCTCTGAAGGCCCCCAC
TCCCTGGGATGCTACAATCCAATGATGGAAGATGGCATTAGCTACACCAC
CCTGCGCTTTCCCGAGATGAACATACCACGAACTGGGTACTGAGGGTACC
AGGAGGGTGACCCTGCACCCTGGGAGGGAGGCGGGAGGAAAAGCTCTGTC
CCGCCTGCCCTCTTTGTGCCAGGCCCATGCCAGGCACCCTGATACATGCT
CTGCCTCATTCCCACTCGGCAACAAGCCTCTGGGTAAGGGCCAGTCTCCA
GAAGTGGACCTGCTCCTTCAAGTCAGCAGCACACATGCCCAGTCCGTTCT
TATCACAAAGATCAAGACCTCATGATACACACCCACGCCTACTCCCTCCA
CGCACATTTGCACGAGCTCCTACTGTGAGCTTTGGGCACTGGGGAGGCCA
CAGGTTTTACATAAGGGGCTGAGGTTGGGGTGCTGTTGGGGGCTCTGGGT
GTTGAGAGGGAGGAGAGTTCGTGGGAGATGCTTCATGCGTGGTCGTCTAT
CTGCCCTGTCTCTCAGAGATGCAGAGTCCTCAGAGATGCAGAGACCTCCC
CCGGACTGCGATGACACGGTCACTTATTCAGCATTGCACAAGCGCCAAGT
GGTAAGGAGGGTCTCCCCAGGTCTCCCCAGAGGGGCTGTGGAAGGCTGGG
GACAGGGCCTGGCCTCAGTGGTGGGTCCCACATAGGAAGGAGTTGGGTAG
GCATCCGTGGTGGCAGAGGTTGGGTGTTGGAGACGGTTGCTCCGGCAGAG
CTGGCCAGAGCCAGGCAGGTACCTCCAGGTCCTGGATGCCGGCCACAGCC
AGTTTCCTGACACGAGGACACCCGCCTGGGCTTTTGGACCCCCGGGTGGA
ATGAAGGAGAGAATGCGGAGAAAAGCGGCGGTGGAGGCTGGCGACAGTGG
GGCCAGGCTAACCACCATGCGGTTTTCTCAGGGCGACTATGAGAACGTCA
TTCCAGATTTTCCAGAAGATGAGGGGATTCATTACTCAGAGCTGATCCAG
TTTGGGGTCGGGGAGCGGCCTCAGGCACAAGAAAATGTGGACTATGTGAT
CCTCAAACATTGACACTGGATGGGCTGCAGCAGAGGCACTGGGGGCAGCG
GGGGCCAGGGAAGTCCCCGAGTTTCCCCAGACACCGCCACATGGCTTCCT
CCTGCGCGCATGTGCGCACACACACACACACACGCACACACACACACACA
CACTCACTGCGGAGAACCTTGTGCCTGGCTCAGAGCCAGTCTTTTTGGTG
AGGGTAACCCCAAACCTCCAAAACTCCTGCCCCTGTTCTCTTCCACTCTC
CTTGCTACCCAGAAATCCATCTAAATACCTGCCCTGACATGCACACCTCC
CCCTGCCCCCACCACGGCCACTGGCCATCTCCACCCCCAGCTGCTTGTGT
CCCTCCTGGGATCTGCTCGTCATCATTTTTCCTTCCCTTCTCCATCTCTC
TGGCCCTCTACCCCTGATCTGACATCCCCACTCACGAATATTATGCCCAG
TTTCTGCCTCTGAGGGAAAGCCCAGAAAAGGACAGAAACGAAGTAGAAAG
GGGCCCAGTCCTGGCCTGGCTTCTCCTTTGGAAGTGAGGCATTGCACGGG
GAGACGTACGTATCAGCGGCCCCTTGACTCTGGGGACTCCGGGTTTGAGA
TGGACACACTGGTGTGGATTAACCTGCCAGGGAGACAGAGCTCACAATAA
AAATGGCTCAGATGCCACTTCAAAGAACCAGTGAACTCTTTTCAGTCTGT
GGAGTCAGAGTGGGGATGAGGAGAGGGGAAAGTCTTCATGGGGCCTGGGG
GGAAGGAAATGGAGTCACAGTGGTTTACTGATCAAACCAGCAGATTAGAG
AGTCTGGTGTCTTGTTAGGAAAAGCATACTGGCTTTGCAGACAGACAAAC
ATGAGAACTTGAATCTTGATTCTTCGGCTTAACTAGCCCGGTGATCTTGG
CAGGGCTCGCCAGCTTGCCTGAGTCTCGGCTTCTCCATCTGTAAAGTGGA
GAGGGAAAGGCCACTGCCTACCACGCAGGTGGCCCACGATAGGAAGTGAG
GCATCGTCTTCTATATGCTGGAGCCACATTGAGACCCAGCACAGAAGCCG
AAAGTCCCCCAGAAAACCAACCTCCATCCTTTATTTGCATTTTTCGAAGA
ATGTGAAAAGGGGAAGCCGATTCAAAACAGCTCTTCCCAAAAGTGACGCT
ACCCAATTCTTGCACAACCCATAGCTGCCAGAGAAAGTCTTGAAGACCAG
CCACCCTGAGAAACCTCACAGCTTTTCTGTCATTCGAGGTCCTCTGCACC
CGTTCTCTAATTCTGTTCACTGATTCAGACTTTCCAGTTAGGTGCCCCAG
CTCCCCTCTCCGCTTCTCGGGTTTGATCTTTCTCCACCCATTCACCTGCT
TCTGCAAAGGGGCTAAAGCCCATTCTCCTTAGAGGGCAGGGGTGTAATTA
TCTGATCATCAGGGAGATTGGCTCCTGGCACGTGGCAGGTGTCCCCAAAC
TGCCACCTTCCTCTCCCACCTTCCTTTCCCTCCCATCTCTGCTCAGTACC
CGGACGGTAACTCTAGGAATTATGGTTCAGGGAGGCCTTGATCGCCCCAT
CAATCCACTTTGTCCCTGGTTTGATTCTTGTGAGCACCATTAACTTTTCT
GGAGGAAGGTGAGGCTTTAAGAACTGTTTAAAGCCCAGGCG
Seq. ID. No: 84 FFAR4 promoter 1
AGGAGGTTAATGTTGGAGAAGGCCTTTTAGTGCAAAGAGCCCACCCTGG
T
TGTACATTCTAGGGAAACCTCTCTGTGTCTATCTCATCACATCAGCCTGT
GATGTAATCAATCTCTCTCCGTCTCTGTCTGTGCTTTTCCCTCTTACTTT
CTCTCTCTCTCTCTCTCTCTCTCTCTCACACACACACACACATACATATA
CCTATAGTTTCCTTTCCCTCACACAAACACTAATATCCCCTGACCCTAAT
CAGACAGTTGCCAAAACCCCATTTGTCTGAGTTCCCTTTATTTCAATTTT
TTGCCCTTTTTCAACTGTTTCAGGAAGCAAACCAGTTCTGTTTTTCAACT
TGAACCTAGTCAATGTTCAAGCTGAGCATCTACTTCTCAGGTTAGAAGTC
ACATCATCAGCTCAGACCTGTTTGATACCGTGAAAAGCAGACACACAAAT
GGACCCCCTCCCCAGACAGTACTTCAGACTCTGGCGCAACCAGCCTGAAG
TACTCAGGTTTCTTTCTTTCTTTCTTTCTTTTTAAGATGGTGTCTCGCTG
TGTGGCCCAGGATGGAGCGCAGTGGCCTGATCTCGGCTCACCGCAACCTC
CACCTCCCGGTTTCAAGCAATTCTCCTGCCTCAGCCTTCCAAGTAGCTGG
GATTACAGGTACCTGCCACCATGCCCAGCTAATTTTTGTATTTTTAGTAG
AGACGGGGTTTCACCACGTTGGCCATGGCTGGTCTGGAGCTCCCGACCTC
AAGTGATCCACCCGCCTTGGCCTCCCAAAGTGCTGAGATTACAAGCATGA
GCCACTGTGCCCAGTCAGTGCTTAGATTTCTTAAGACCCAACCTTAAGAA
AGACAATTAGAAGACAGAGTGGGCAACTGGGAGAGGCCTGGTAATATGGC
AGAGGGAAGAGGGGGTGAAGGTGTGGGTGGCAGGAGGGTGGCTGGTTGGG
GAAGTGGCCACTGGATCTTAGGCCACAGGCATACTTTTCCATCTCCAGCT
TCCTCATTTACAGAAAACGAGACTGCAGTGGTGTGATGGAAATGTGTGGG
ACGGGGACAGGTGACTGGCTTTCATTACTAACCAGAGGACAGCTTAGACA
AGTCACTCTGGCTGGGCCTCAGTTTCCTTATCTCTAACGGGACTGGATTG
CCCAGTGCTCCTCAAACTGTGGCACCTGGGGCACATTCATAGCTTGTGTG
TGAATCTGCCAGCCTTGAAATATCTGTCTTCCTTGGATGGCAAGTTTATT
AAAAATTTTGGAAGGACTCACTCTTTTTATAAGAAAGCATTTATGTCATT
GTTCAGAATGCAAAATGTATATGCATTTTAGTTACAATAACTAATATTTG
ATAACATTTAACGAGTAATTACTGTGTGCCAAGTGTGCAAAACTCTAGGT
GAGCCTCACAATAACCTAATGGGGTATTATTATTCCCATTTTATAGATTA
AAAATCTGGGGTAGAGGGAGTAGATAATCCACCTAAAGACCTGTTATTAA
GTAGTGATAAAAGATCTCCACTCCCTCTCTCCCCCACAAAAAATATGGAC
TCATTACAAGTTGTTTTAGGATCTACCTCCAGACCCATGGAGTTTCTTTA
GTAAAGCCTGAACGACACAGGCCAAAATAATCTCCAAAGGCCAGCTCTGA
CCCTTTTAAATCAATTTTAGCTAAATCCGTTCACAAAAGGCTTCGCACAT
CCAGTGTCCCTGAAAAATAAAGGAGGTTGGGCAGGCCCTGCGGGGGCTCG
AGGAATTCGCTAAGTGAGTTTTCTGGCTTCTGGATACACTTTCAAAGGGC
CAGAGGGCACGAGGCTTCCGCCTTGGCCGCCACCTCCCCGGCCAGCTGCG
GTGTTCGCGGCCAGTGTTGCCGGGCACTTCCTGGTTCCCGCGCGCCCCGG
GTGCAGCTCCCTGCACCCAGTGCTGGCGCTCCTCAGAAGGGAGGGGGCCA
GAGGCGAGATGTCGCAACCGCCTCCCTCCCTCTTTCCCCGCCTTGGCACT
CAGTCGCCTCCCAGATGAGCACTCTCTCAGACCGCTGCGGGCCGCCAGGC
GCCGGGAATGTCCCCTGAATGCGCGCGGGCAGCGGGCGACGCGCCCTTGC
GCAGCCTGGAGCAAGCCAACCGCACCCGCTTTCCCTTCTTCTCCGACGTC
AAGGGCGACCACCGGCTGGTGCTGGCCGCGGTGGAGACAACCGTGCTGGT
GCTCATCTTTGCAGTGTCGCTGCTGGGCAACGTGTGCGCCCTGGTGCTGG
TGGCGCGCCGACGACGCCGCGGCGCGACTGCCTGCCTGGTACTCAACCTC
TTCTGCGCGGACCTGCTCTTCATCAGCGCTATCCCTCTGGTGCTGGCCGT
GCGCTGGACTGAGGCCTGGCTGCTGGGCCCCGTTGCCTGCCACCTGCTCT
TCTACGTGATGACCCTGAGCGGCAGCGTCACCATCCTCACGCTGGCCGCG
GTCAGCCTGGAGCGCATGGTGTGCATCGTGCACCTGCAGCGCGGCGTGCG
GGGTCCTGGGCGGCGGGCGCGGGCAGTGCTGCTGGCGCTCATCTGGGGCT
ATTCGGCGGTCGCCGCTCTGCCTCTCTGCGTCTTCTTCCGAGTCGTCCCG
CAACGGCTCCCCGGCGCCGACCAGGTGAGCGCCCCTCTGTGTGTGCCGGG
CAGGTGTCCTGCGCAGGCTGGGAAGCGGGGCCCCGACGGAAGCTGGGATG
AGGATGATCAAGAACAACAATAGCCATTTATTGCACTTAATCGTTGTGCC
AAATCTTGTGCCCATGGCTGTGAAGTTTAATCTCTTAAATCTCACTACAA
CGCTGTGCACACGCCCTCCTAAATGATGTAAGTGGAGTCCCCCAAATTCT
TGCAAAATGCAATGACTGTTGCGAGGTTAATTAACGAGTAGTTTAGGAGC
GAGACGGAACTTTGGGGGTGCAGGGTGGCCAAACACTTTGTATTGAATCA
TGATTCCTCGCCAGGTGCTACAATACTGTTATTATCACACCCATTTCACA
GATGAGAACCAGAGGCACACCGAAGTGTATAATAACTTGCCCAGAGTATT
TTATCCGTAATTCGAGGAGGAGATGGGCTCCTTCCAGAAGTTTACCCGTA
ATTCAAGGAGGAGTTGGGCTCCTGTCCAGGGTTGGGTTATGGTCCTGCTT
TGAAAGCGCGCGGACAGGCATGTGAGACCCGGGGACCCCAGATGCAATGC
TGTCTTTAGGGGACTTGTGACAGAATTCCCTTCCGGGGTCTTCAGTTTTT
TCAGCTGCAAAACGGAAGGATTACACTAGACCTTCGAGGTGTCCTGGGCG
CCTGAAATGTGCAGATTACAGAGGCTGGACCGACGAGCTCTGTTTTACAT
AGCGGGTCAATAGTTGTCATTATTAGCTCATAGGGGTGGGAGTGACCATG
ATCGGAATAAGGTTCTGCGGATAGTGCAGGACATTGGCTCTGTTCCCCTC
AAGTCCCGCGGTCCGGAATCCCGAGCTCGGGTCTTTTTCTCTTGCCGCTT
TGCCCGGTGGTTGCAGGGTTCGTGCGCCGGGATCCACACGGAAGAGGGAA
TCTAGACGCGGAGTCACGGTGGAGGACAGTGGCCGCCGTCTGGCTGTCTT
CCCAGTGTGTTTGGTTCTCCATGCCTCGCGGAGAGAGATAGAGAAAGGAC
TTAGGTCTGTGCGCTGGGGGCTCAGCCTCCAGGAGCTTCTGCCCGTGGCT
GGGTTCTTGGCCTCCCCTTCCCTGCCGGGGTCACTGGTTTTCCGGCCTGG
AACCCCCTACCCTTAAGCTCCCTCTCCACCTAGGAGTGAATATGGGGCGC
CCTGAGTAATCGTGGCACAAACAGGAATCACGGTCCCAAAGAGGGACCTT
CGAAGTGGTCAGAGAGGGGAGTAGGGAGGGAGAAATGACATGTCACCAGG
GGAAGGCTTGGTTATGGAGATGCACACAGCCCACCAGGGACACCCCCTGA
CTGTGTGACCCTCCAGAGCCTGAGACAGAGAGGTGGAGCTTCTCTGGGGG
T
Seq. ID. No: 85 FFAR4 promoter 2
GGTAGGAAGTGGGCAAGAGCTGCTATGGCCTCTTTGCCTCCACTAGGGG
A
TGGATGGGCTTCCTGAAACAAACCCAATTCAGAGAAAAGAGTCAGAGGTG
AGGACAGGTGCCCAAATAAGTTGTTGTTGTTTGCATCCCTGGATCCAGAT
GTGCCTAAAGCTAAAGTCACTGCCTGGATTTTTTGCTTATCCAGGCCAGT
GCATTTCTTTTTTTCTTTCTTTTTCTTTTCCTCCTCCTCCCTTTTATCCT
CCTTCTCTCCTTTCTTCTCCTCCATTTCTGTTTGTTTTATTTGAGTTGGA
TTCCTGTTAGTTGTAACCCAACGTCTCCCGATCATTATATTCTTCTGCCT
CAGTCTTCTCACCTGTAAAATGGGGATGATAATAATAACAATGTTCTTCT
TCTGCACAGGATTGTTGTGAGACTTAGCTGAGTCACCACAAGTAAGGTGC
TTGTTACATAGTGAGAGGTCAGTAAATGTTACCTATTAAGGAGTTGGAAA
AAGCTGAATTCAGAGAATTTTATCTCCCTGCTGGGGGCGGTGGCTCACAC
CTGTAATCTCAGCAATTTGGGAGGCTGAGGCAGGTGGATCATCTGAGGTC
AGGAGTTCAAGACCAGCCTGGTCAACGTGGTGAAACCCCGTCTCTACTAA
AAATACAAAAAATTAGCCAGGCATGGTGGCACGCGCCTATAATCCCAGCT
ACTCAGGAGGCTGAGGCAGGAGAATCACTTGAACCTGAGAGGCGGAGGTT
GAAGTGAGCCAAGATTGCACCATTGCATTCCAGCCTGGGCCACAGAGCGA
GACTGTCTCAAAAAAAAAAAAAGAAAAAGAAAAAAAGAAAAAAGAAAGTT
TTATTTCCCCTCCCCCGCAAAAAAGAAAGAGATTTCTCCATGCATCAAAA
TATGAGGAATAGATTCCTATAATGCAAATTATGAGAAAAGAGAAAGCACT
GTGTCAGTCAGTTCACCAGGGAGTCATTATTCCAGAATTTATGGCCTAGG
AGTCACTGACCTCACCTCACGCCTCTGTCTTTCTCTCTTTCTCTCACTCT
CTTGCTTGTGTGTGTGTGTGTGTGTGTGTGTGTATGTGTGTGTTTCACAG
GAATTGCGAGAGAGGAGGAGAAAGGGGAGATGAAGTGGTTGGCTGGACAG
AAATGGCTGGTTTTGCAATGCAGTTTGGGGAGAGGAAAAGTAAACTGGGA
GAAAGTTATCCAGGCCTTAGAGCGTCCTTCCTGCATGGGTTGTAGAAAGA
GAGGACATGGGGCTTGCCATTCGGAAGCTGCTCAGCTAAGTCTGGTTATT
TGTCTGGGGAGTCAGGAGTCTAATGAAGCCAGGCCAGAGGCCTCTAAGGC
TGAACAAAACATTAGCACAGGAGGAGCAACAGTTACACACACACACACAC
ACACACACACACACACACCCATTCTGGCTGGATTAAACAGAAAAAGGTAT
AATGTAATTCACAGGCTCTCCGATGGGAGGGCCAGAGAATTGACCGTGAA
GACTTCCCAGCCTGGAACGATGTCACATGATGAAGGCTGCTCCAGTGGAA
CACTCAGTGTCACCACACTACAGCACAGACACCATGCCTGGCTTGCACCA
CTGGTTCTGGATGTGAGATCCTAGAAGCCTCTTGATTGTGCCCTAAACTC
TTGCCCATAAATGGATTCTGTGAAACCCTAGCTTGTCCTCTCCTGCAAAG
GCAAGTCTCTCACGGTATGTCTGGTGGTCTAAGCTTAGGGTACATGCCTT
GGTGCAAGGGAGATGGGAAAGCCAGTGTTGACCTTTCCCTTGGGGAGGCA
GGACTCATACTGTGGGAAATTTGCAAGTCACCTGATAGAGTTCAAAAGGA
CAACCATCAAGTCTGATATATATCCCCACAATAGCATTGCTATATTGGGT
GTGTGTGTGTGTGTGTATGTATGAAATAGACAAATGATCTATATCTCAAG
GTCCACTTTCCAGGATGGTTTCATCCTTAAAAATCCAAAGAACCATCATT
CAAGCTGCCACCAGGAGCAGGGAGCCCAGTTCCCTCATTTGGGTGTCACT
CAATACCTCTGAGTCCAGACAACTGTTCCAAGCTCCAGGTACCGTCTGGT
CACCCCTTCTCCTTGTGGCTCTGCTTGTTCAGGTCAGCATTGCCCCCATA
TGTGGGCCCAGGTCCCTTTCTCCCCTTACCCCCGAAATGAAAAGCTGACA
CACTCTTCAGAGATGTACCAGGCTCATGGATGGATGGACGGATGGATGGG
CGTATAGGTGGATGAGGGGGTGGGTGGATGGACACCAGAGGTTTGAGGAC
AATGGTCACTGTAGCCAATGACACCAGGGGGCAGTGCGGAGCAGCTTTTC
AAGGCCTAACTCCCTACACTCTTCCATCAGACCTGGCTGGCAGGGCTTGG
AGAGCTCATTTTGTTCAACATTTTCATTTGTGAGGAAGTTGTTGAGGCCC
AAGACAGGAATGGCTGTGTCAGTAGCGCACAGCAGCTGAGCTGGCTCTTG
ATGCGGCTCCAGCACCCTCTGCATTACAGTATTTACATAAGCCCACCTTC
CCAGGCTCGGAGGGCCCCCACGCTGCCAAGCCTCCGTCAGCTATCAGTGA
TTCTGGAGCCCTTCCTAGGAGTCTCATCGGTTGCCGGCTGCTGAACTCAA
TCAGCATAGAGGAAACAGACAGTCAGAGTTCAGGGCGTTGGCCAGGCTGA
GTCATTGTCTTCGCCCCTCCGAAGGGCTGCATGTTAGGGCCCTCACTGGA
CACCCCTTCCCTAGCCATGTCTTTTCTCTCCTTTTGGAATCCAGATCACC
TGGAGGTCATATTTCCCAGGAAAGGAAGGACCCCACGCTGAGAACACATC
TTACAATAAATGATCCCTCTCTTCTCAAAATTTACGTCTGTGTTACATCC
CCTTTAGCCCTGCTTTTTAATTTCCACTGCCTGTTTCTTTGGGGGCAGTG
GGGGTGGAGTGGGGGAGTACCAGGGTGGTGATGGCCCCTCCCACCCCCAC
CCCTATCCTTATCCATCTTTACAGCAGGCTCCTGAGGACCACGACTTAGA
TAGCAGAGACATCAAGAGGCTCACTAAACCTCAAATTTCCATTTTCGTTC
ATTCATCTATTTAATTATTCAACTATTGAAAATATTTTAAGCCCCTCCTA
TGTGTCAGATGCTAAAAAAAGTCACAAACTTACACTTCTTAAGCCCTTAC
TACACTAAAGGTGTGACATTTAGTATATTATCTTACACAAGCCAATGAAG
GAAGTATTTTATTTATTTATTTATTTTTATTTTTTTGAGACGGAGTCTTG
CTCTGTCACCCAGGCTGAAGTGCAGTAGCACAATCTCAGCTCACTGCAAC
CTCCACCTCCTGGGTTCAAGCGATTCTCCTGCCTCAGCCTCCTGAGTAGC
TGGGATTACAGGTACCTGCCACCCATATCATTCAATTCACAAGCATTTTA
ATGACTGTGTAGTATTCTACTGAAGACATATAACTGATTTACCTCCCCCT
TACTGTTGACCTTATATCTATTGTAGAAGTGGTTTAGTGTTTCAGTGTTT
AATATTTTCTTTCTTTCTTTGTTTCTTTTTTTTTTTTTTGAGACAGAGTC
TCGCTCTGTCGCCCAGGCTGGAGTGCAGTGGTGTCATCTCGGCTCACTGC
AACCTCCGCCTCCTGGGTTCAAGCTATTCTCCCTGCCTCATACTCCCGAG
TAGCTGGGATTACAGGTGCCCACCACCACGCCCAGCTAATTTTTGCCATG
GTGACCAGGCTGGTCTCGAACTCCTGACCTTGAGTGATCCACCTGCCTCA
GCCTCCCAAAGTGCTGGGATTACAGGCGTGAGCCACCGTGCCCAGCCTTG
AAGGATGCATTGTTATTTTCGTTTTACAAATGAAGAAACAGAGATCTAGA
GAAGTGATGTCACTTGCCCAAGAGTGCACAGCCAGTGAGCTACAGGACAG
GAATCAAACCCAGGAGGCCACACCCAAGTATCAGACATTTCCTGCAAGTG
T
Seq. ID. No: 86 FOXO1 promoter 1
CCATGCTCAAGAATAGGGTGGCAAGGCAGGAAAAGACTCACACATCCC
A
GCCCCCGGCCCCTCAGGGCAGCCTCTGTGTTTCTAACTAAAAGCCTCCTA
CCAGGGGCAAGACCAAGTTCGGGCGCTTCCCCGCATGGAGAAACGCTGGC
CCCACGGCGCCCGGCCCCGACGCGCTGGCCCTTTAAAGGCGCGGGCTTCC
TGCGCCCGGGGCCCCTCCCACGCCACCGCCACCGCCACCGCCACCGCCCG
CGCCCGCCCCCGCCGAGGGTCACACTTGAACTTTAGCTCGCCCCGCGCGG
CCAGGCGCCGGCGCTCCGCAGGCGGTGGCCGCCCCTTCGCACGTGTCGGA
GGCACCACCTCGGGCTCGGGACGCCGGGCCCCCGGCACTCGAGGCCACTC
CGGAGAAAACCAAAACAAAGCAAAATGCGCACCGCCTCCAGAAACTGCCC
GGCAGGCCCTGCACCGTCCCCGGCCGGTCGCGCCCTGCCTGTCCCCTCCA
CCTGCAGCCCGGTCCCGGGGCAGCGAGGCTCCGGGGCCCCTCGCCGCGCC
CTCCCCCGCCGCGCCCGGCGGAAACAGCTCCGCGGCGCGGCCGGAGAGAA
CCCGCCCTCCCCCCGCGGAGGTCCGGGAGGGAAGGGGCAGCCGAAGCAGT
CGGCGCGGGCCGGGGGTTGCCGCTCCCAGCGAACCCCTTTCTCCTTTCAC
TGGCAAACTTTTCGGCCTCGCTCTGACGTCCACTTCTTGGCGCACTTTCT
TTACTTAGTTCCCCAACGAGCCCCTTACCGCGTCCCACGCGAACTCCTGA
CTGGCGCGCACGCACACCTACTGCCGTCCCCGACCGGACCCGGGCGAGGC
CACCGCGACCACCGCTTCTCGCCCGCCCTCCTGGGAACGCGCTGCCCTCC
TGCTCCGCACCTTCAGGCCGAGCAAACCTGCACAGCTGCGCCCTCGCCTG
ACCCACCGCGCCCCCAAGGTCCGGCCGCGCGCCGAGTCCACTCACCTTCC
AGCCCGCCGAGCTGTTGCTGTCACCCTTATCCTTGAAGTAGGGCACGCTC
TTGACCATCCACTCGTAGATCTGCGACAGCGTGAGCCGCTTCTCCGCCGA
GCTCTCGATGGCCTTGGTGATGAGGTCGGCGTAGGACAGGTTGCCCCACG
CGTTGCGGCGGGACGAGCTGCTCTTGCGCGGCTGCCCCGCGAGCGGCCCA
GCGGCGGCGGGGGGCACCGGCGGGTGCTGCGACAGCGGCCCGGGCGGCGG
GGGCTGCGGTGGCGCTGGGTGCAGGCAGCCCGCCTCCGGGCCCTGGAAGT
CCCCGCACAGCCCCCCGGTGGCGGCCGCGGCGGCCGCCGCCGCCACCGCC
GCCGCCACGGAGCCGGGCGCCTGCGGGAAGTCCTCGCTCTCCTCCAGCAA
GCTCAGGTTGCTCATGAAGTCGGCGCTGACAGCGGCAGCCGAGGCCGAGG
GCAGGCCCGCCGCGGCGTCGGGGTTGGCAGCCGCGCTGCCCGACGGCGCC
GGGCTGGAGGTGGCCGAGTTGGACTGGCTAAACTCCGGCCTGGGCAGCGG
CCAGGTGCACGAGCGCGGCCGGGGCAGCGGCTCGAAGTCCGGGTCGATCT
CCACCACCTGAGGCGCCTCGGCCATGGTGACCCCCGCCCCTCCCCCAGCC
GCAGGAGAGCCAAGAGGGGGAGAACGCAGCACTGGGGGCGGACGGGGAGG
GGGCGCGAAGGGACGGTCCGAGATTTGGGGGAACGAAGCCGGTGCGGCGA
GCGGACGGAAACTGGGAGGAAGGCGCGGCGGAGTGGAAGCGCGAGCCCAG
AACTTAACTTCGCGGGGCCATCCACATCGAGGCTCCTCGGGGTCCGCCGC
ACGGACTGGACGGCCGGCCAGAGCCGCCGGGCCGGGGCAGAGCCTGCGCC
GCGCTCCAGCTGACAGGGCCGCGGACGGAAGGACGGACGGACGCCGCGGG
CCGCTTGCTCTCCCCAGCGGCGCGCCCGCTGCGCTGCTGCCTGTTGAATG
TGGCGGCTGCGGCAGCGGCTGCTGCGACTACCAGGCCGCCCGACTTACGG
GATCTGCCGCCGCCCCCCGCCCGCGGCGGCGCGCGCGCCGGCCCGCCCCT
GACCGACAGCCCGCGCGGCCAATGGGCATGCGGCACCGCCGCCCGGGCAG
CCAGTGGGCGCCGGGCTGGGTGGGGCCCGGTTTTCCACGGGGAGGCGGCG
GTGGGCTGGTGGGGGGTAGTGGGGTGTTTTTCTCTTTCACACACTCACCT
CCTTTTTTTTTTTTTGGATCTCTATTATTTTCTGGTAATTCTCGAGTGTT
TCTGTGATTCTCTCGCCTTCTCAGTGTTTTGATTGCTAGGAAGCAAACCA
GCGTGGAGGCGCCGGCGACACTTTGTTTACTACGGAGCAGCAGAGCCGAG
TACTCGGGAAGCCCGGGTGGGAGGAGGCGCTCGCTGCTCCCTGACCTCCG
CTGCGGGCCGAGCCCGGCGGGCTGGCAGGGCAGGGGGCCGAGGGCCGGGG
GCGCGGGGTGGGCGGGCGGAGGCGGCCGCGAGGAATTCTACTCAATCGCT
CCCTCCTGGCTCCACCCACGATGTCTTTGCTGAACGACGTGGGGAATCGG
TGGGTTTTGTTTTGGTTTAATGTTTTCTTTCGCTGCGATCTGTCAAGTCC
TCCGGCCCCCTCGCGAGCGGCACACGCCCCCCACCCCCGGCGCCGCGGCT
CCTGCAGTCGAGTCCGCGCCGAGGGACCCTTCTCCGTGCCCACCGGTCCG
CACCCCCGGGCTCAGCCTGTGCCATTCGGTCTAGCCAGAGGCGCGTCACC
CAGCCGGCAGCCCCCAGCCGGATTCACTGTATTCTTGACCTTTTTTAAAA
TGCTAGAAATGAGGAACAGGGGCTCCGGCGCGGGGAGGAGATTATCTGGC
CTCCCTGAACACACGAATCCAAAGGCACCCGCGACCGCATGCCCTCCGTG
GGAACCTCTTCCCCTGGGCGGGAATCTCCCGCTCCGTCCACTAAGTCCAG
GGCCGGCAGCGAAGGGCTCAGGAGCGCAGTTAGAGAGAACCAGGTCATTT
TATTACTTATCCCTAATTAAATTTAGCTGTTTGTTACTTCTTTTTTATTT
GCAACGGAAGTGATCGGGTGTTAAGTAAAAACGCAAGGGGGGCGGGAGAT
AGGACCAAAGCCTTGGGGCGCAGCCTGCCCCACCCCCAAATTCCTTGAGG
CTACTTGCTGTGTGTGTCAGTCAACCCCCCACCCACACCCCACCTTTCCT
CATCTGGGCTGCAGGTCCAGGACATCCTGTCAAGGATCAGTGGGGCCAAA
GGTGGGAAGATAATGGCCCTTGTGTCTCAAGCAGAAGAAACAGTTTTCCA
GTTTTGTTTTGTTATGCTTTTGCTTTTTTAGGAAGAATATGGAAGGCATT
TCCTTTTAGAAAGATGGGACCACTTAGGATGGAAGATGGACAGTAGGGTT
TTAAATATCTTGTCCAAAAGGACAGCTTCCAACTCTTGTTTTTTAATCCC
TTTCTTTCTTACAAGTAATTCTGCAATGGAAAAAGTGGCATCTAGAGAAA
ATTAGTCAGTTGGACAAGAGGTTGTTGAAAAAATAAAAGAGAAAAGAGGG
GAAAAAAGGAAAAATTAGGCCTCTTACAAAAGTTAAAAAGATTGAGAGAG
ATTATCTTTCTTCTTTTATATTCTCACTATCGTCTCTCTAGTCCGGGCTC
CTGTTTCTTCATAATCTATTTATCACAACAGCTTCTTGCTGATACTAGGC
ATGGGTGGGGTGGGGAGCGTGAGCCGGATGGATTGGGGACGGGCGGGGAC
TGGTTCCCACCCTGGTCTTGTGGTCTCTTCACGTTTACTATCAAATTTGG
ACAGTCTGGGCCTCCTCTGACAGCCGCTAGGGGACCTCAGTAACTCAGAT
GATGAGCTCAAAGGCAGGCCCCCAAAGTACAGCTGTTGCTTCCCCCCACC
TTTCCAATTACACTCTGCAGAAGCAGCCCCCGATCCAGACAGCCGCACCC
T
Seq. ID. No: 87 FOXO1 promoter 2
CCGTCCCCGGCCGGTCGCGCCCTGCCTGTCCCCTCCACCTGCAGCCCGG
TCCCGGGGCAGCGAGGCTCCGGGGCCCCTCGCCGCGCCCTCCCCCGCCGC
GCCCGGCGGAAACAGCTCCGCGGCGCGGCCGGAGAGAACCCGCCCTCCCC
CCGCGGAGGTCCGGGAGGGAAGGGGCAGCCGAAGCAGTCGGCGCGGGCCG
GGGGTTGCCGCTCCCAGCGAACCCCTTTCTCCTTTCACTGGCAAACTTTT
CGGCCTCGCTCTGACGTCCACTTCTTGGCGCACTTTCTTTACTTAGTTCC
CCAACGAGCCCCTTACCGCGTCCCACGCGAACTCCTGACTGGCGCGCACG
CACACCTACTGCCGTCCCCGACCGGACCCGGGCGAGGCCACCGCGACCAC
CGCTTCTCGCCCGCCCTCCTGGGAACGCGCTGCCCTCCTGCTCCGCACCT
TCAGGCCGAGCAAACCTGCACAGCTGCGCCCTCGCCTGACCCACCGCGCC
CCCAAGGTCCGGCCGCGCGCCGAGTCCACTCACCTTCCAGCCCGCCGAGC
TGTTGCTGTCACCCTTATCCTTGAAGTAGGGCACGCTCTTGACCATCCAC
TCGTAGATCTGCGACAGCGTGAGCCGCTTCTCCGCCGAGCTCTCGATGGC
CTTGGTGATGAGGTCGGCGTAGGACAGGTTGCCCCACGCGTTGCGGCGGG
ACGAGCTGCTCTTGCGCGGCTGCCCCGCGAGCGGCCCAGCGGCGGCGGGG
GGCACCGGCGGGTGCTGCGACAGCGGCCCGGGCGGCGGGGGCTGCGGTGG
CGCTGGGTGCAGGCAGCCCGCCTCCGGGCCCTGGAAGTCCCCGCACAGCC
CCCCGGTGGCGGCCGCGGCGGCCGCCGCCGCCACCGCCGCCGCCACGGAG
CCGGGCGCCTGCGGGAAGTCCTCGCTCTCCTCCAGCAAGCTCAGGTTGCT
CATGAAGTCGGCGCTGACAGCGGCAGCCGAGGCCGAGGGCAGGCCCGCCG
CGGCGTCGGGGTTGGCAGCCGCGCTGCCCGACGGCGCCGGGCTGGAGGTG
GCCGAGTTGGACTGGCTAAACTCCGGCCTGGGCAGCGGCCAGGTGCACGA
GCGCGGCCGGGGCAGCGGCTCGAAGTCCGGGTCGATCTCCACCACCTGAG
GCGCCTCGGCCATGGTGACCCCCGCCCCTCCCCCAGCCGCAGGAGAGCCA
AGAGGGGGAGAACGCAGCACTGGGGGCGGACGGGGAGGGGGCGCGAAGGG
ACGGTCCGAGATTTGGGGGAACGAAGCCGGTGCGGCGAGCGGACGGAAAC
TGGGAGGAAGGCGCGGCGGAGTGGAAGCGCGAGCCCAGAACTTAACTTCG
CGGGGCCATCCACATCGAGGCTCCTCGGGGTCCGCCGCACGGACTGGACG
GCCGGCCAGAGCCGCCGGGCCGGGGCAGAGCCTGCGCCGCGCTCCAGCTG
ACAGGGCCGCGGACGGAAGGACGGACGGACGCCGCGGGCCGCTTGCTCTC
CCCAGCGGCGCGCCCGCTGCGCTGCTGCCTGTTGAATGTGGCGGCTGCGG
CAGCGGCTGCTGCGACTACCAGGCCGCCCGACTTACGGGATCTGCCGCCG
CCCCCCGCCCGCGGCGGCGCGCGCGCCGGCCCGCCCCTGACCGACAGCCC
GCGCGGCCAATGGGCATGCGGCACCGCCGCCCGGGCAGCCAGTGGGCGCC
GGGCTGGGTGGGGCCCGGTTTTCCACGGGGAGGCGGCGGTGGGCTGGTGG
GGGGTAGTGGGGTGTTTTTCTCTTTCACACACTCACCTCCTTTTTTTTTT
TTTGGATCTCTATTATTTTCTGGTAATTCTCGAGTGTTTCTGTGATTCTC
TCGCCTTCTCAGTGTTTTGATTGCTAGGAAGCAAACCAGCGTGGAGGCGC
CGGCGACACTTTGTTTACTACGGAGCAGCAGAGCCGAGTACTCGGGAAGC
CCGGGTGGGAGGAGGCGCTCGCTGCTCCCTGACCTCCGCTGCGGGCCGAG
CCCGGCGGGCTGGCAGGGCAGGGGGCCGAGGGCCGGGGGCGCGGGGTGGG
CGGGCGGAGGCGGCCGCGAGGAATTCTACTCAATCGCTCCCTCCTGGCTC
CACCCACGATGTCTTTGCTGAACGACGTGGGGAATCGGTGGGTTTTGTTT
TGGTTTAATGTTTTCTTTCGCTGCGATCTGTCAAGTCCTCCGGCCCCCTC
GCGAGCGGCACACGCCCCCCACCCCCGGCGCCGCGGCTCCTGCAGTCGAG
TCCGCGCCGAGGGACCCTTCTCCGTGCCCACCGGTCCGCACCCCCGGGCT
CAGCCTGTGCCATTCGGTCTAGCCAGAGGCGCGTCACCCAGCCGGCAGCC
CCCAGCCGGATTCACTGTATTCTTGACCTTTTTTAAAATGCTAGAAATGA
GGAACAGGGGCTCCGGCGCGGGGAGGAGATTATCTGGCCTCCCTGAACAC
ACGAATCCAAAGGCACCCGCGACCGCATGCCCTCCGTGGGAACCTCTTCC
CCTGGGCGGGAATCTCCCGCTCCGTCCACTAAGTCCAGGGCCGGCAGCGA
AGGGCTCAGGAGCGCAGTTAGAGAGAACCAGGTCATTTTATTACTTATCC
CTAATTAAATTTAGCTGTTTGTTACTTCTTTTTTATTTGCAACGGAAGTG
ATCGGGTGTTAAGTAAAAACGCAAGGGGGGCGGGAGATAGGACCAAAGCC
TTGGGGCGCAGCCTGCCCCACCCCCAAATTCCTTGAGGCTACTTGCTGTG
TGTGTCAGTCAACCCCCCACCCACACCCCACCTTTCCTCATCTGGGCTGC
AGGTCCAGGACATCCTGTCAAGGATCAGTGGGGCCAAAGGTGGGAAGATA
ATGGCCCTTGTGTCTCAAGCAGAAGAAACAGTTTTCCAGTTTTGTTTTGT
TATGCTTTTGCTTTTTTAGGAAGAATATGGAAGGCATTTCCTTTTAGAAA
GATGGGACCACTTAGGATGGAAGATGGACAGTAGGGTTTTAAATATCTTG
TCCAAAAGGACAGCTTCCAACTCTTGTTTTTTAATCCCTTTCTTTCTTAC
AAGTAATTCTGCAATGGAAAAAGTGGCATCTAGAGAAAATTAGTCAGTTG
GACAAGAGGTTGTTGAAAAAATAAAAGAGAAAAGAGGGGAAAAAAGGAAA
AATTAGGCCTCTTACAAAAGTTAAAAAGATTGAGAGAGATTATCTTTCTT
CTTTTATATTCTCACTATCGTCTCTCTAGTCCGGGCTCCTGTTTCTTCAT
AATCTATTTATCACAACAGCTTCTTGCTGATACTAGGCATGGGTGGGGTG
GGGAGCGTGAGCCGGATGGATTGGGGACGGGCGGGGACTGGTTCCCACCC
TGGTCTTGTGGTCTCTTCACGTTTACTATCAAATTTGGACAGTCTGGGCC
TCCTCTGACAGCCGCTAGGGGACCTCAGTAACTCAGATGATGAGCTCAAA
GGCAGGCCCCCAAAGTACAGCTGTTGCTTCCCCCCACCTTTCCAATTACA
CTCTGCAGAAGCAGCCCCCGATCCAGACAGCCGCACCCTAAAAATAGGCG
GTGCCCTACTGGATCATTCTTGGCAAAGGAAGCCAGCTTCAAATTTGGCC
TTGCCTTCATTTCTATTCCGGTTTTTCAGGGCGTCAGAGAATATTTATTT
GGTTAAAGGAGTCTTCCTACAGACTTTCCCCAAATCCTTGAAATCTTTGA
GGCTCTCCTGCCCAATTGTTTCTTCAAATGTCATAGTTTACTTTTATTTG
GTCTTTCTTTAAAAAAATTGACGAGGCAGGCACAGAGAACCTATAACACC
AATGTCCCTGACACATAGGTCCAAAAAATTAAAAATATATATTGAAGGGT
TTTTACAGCTAACCACCACACTGTTGTATCTAAATATCAATTTACTGAAC
CCCAGGATGTTGGTGAATATTTCACTTGTTTCCAGTCTTTAGCTATCATA
GACTATTCTGTAAGAAATAACCTTGCAGGGAGGCCGAGGCGGGCGGATCA
T
Seq. ID. No: 88 SLC2A1 promoter
AGTTCAACCACTTACTAGCTGTGTGACTTTGGCAAACTTATTTCCCTTCT
CTGAGCTCCAGGCTAGTGAGTAACCAGCAGGGCTATAAGGTCCGTGACGC
TTAAAGTAGTTTGGCTAAGAACCTGCTGATTTTCTCCTCCACTTTTCTGT
CTCCACGTAGTATTCCCTTATCCACTCAACAAATAAATACCCATGTCTGG
CATAGTATGCTGAATAGATAGTTCCTTCCCTAGAGGAAGTGACAAAAGTG
ATCTCTTTCAGATGGAAAAACTGAGGCCCAGAAGTTAAGTGGCCTGCAAG
AGCCCTGAGATATTTGGACTTCCTTGCCACTAGCTAGAAGAGCTTCTCAC
TGAACTTTTGTTTAGTTATATCCCACCCTTACGGCTCACCTAAGATCCTG
GGAGCCTTCCCCGGGAACTTCCCCGGGGTCTCACCTCAAGCGAGGGCCCA
GGAAGGGTGAACCAAGTCGTCTGGATTTATGGATTCAACAGGTGTGGGGC
GGAAGGCCTTTTCCTGCCGAGGGTGCGGCCCAGCGCCACCCCGCTCAGTC
TCAGCGGGACACCTCCACAGCCAGTAGGAAGGGCAGTTCAAAATTCAGTG
CTACCCCCGCGTCCCAGCTAAGAAAACAAGGGCCATCCAGGTGCCCTCCC
AGCTGACGAGATGGAAAAATCAAAGTATAATAGATAAAGAGGGACCGTTT
CAAAAGAAAAAGGGGACTTGAAAATATTGTAGACTAGGGGGAGGAAAGGA
GGTGGAAAGAAACAATGCCCTACACACAGGAGGGGCTCCACAAACAATTT
GTTGAAAAGAAATCGCTCTGCGGACGCTACAAAGATGCCCTGCAGGACAA
GCCCACTTACACAGAAAAAAAGAAATCCTATCTAGGGAGTTGTTGGTCCC
CAGAGTCCCCATCCCTCATTCCCCAACCCCGTGGCGTGCGCGCTTCCCGC
TAGACTTCAGCCTGGGGCTCTTTCCTGGCCCCCAGATCCCCCGCCCCTCC
GGTTGCGCTCCCCGCCTGGCCCCGAGGGTAAACCTGCCTCCGCGGAACAC
ATCTCCGCGAGACTGGGCCAAAGCTTTGGGGCCTGCACTCCTCGCAAAGG
AGAGCCTCCGAGACCGGAGGTTCATCTCAACCCCCGCGTTCGAGCCCGGG
CCTGGAGAAAAGTGCCCTCCCCTCGGCCTCGGCCTCGTCACCGCGGGGGA
CTGGCGGGAGGAGTAGGGGAGGCCGGTGGGACACCTCGCACCAGGCCGCC
CACCTACAGGGGCGTCCGGGCTAGGGGAGCAGACGGAGAGCGGGGGCGGC
TCCTGACTCCTCCGCGCGCCGGGGCCGGGGCCGGGCCGGGGGCTCGGCCC
GCTCAGCCAATGGGCGCCGCGCTCGGGCCCCCTCCCCCGCACCGGGAGGG
GCCGAGGCTGGGCTGCTGCTGCCGAGGTCGTGTGCGACGTGCCGCTCCCG
GGCCCTGTGTCCCCAGCCCCAGCAGGGCCGCGTGGACGGCGCGGCGCCCC
CGGAAGGAGGCCATGTGCTCAGTGCCGGCGCCAACTACGCACGCGGCGCC
CTCCGGCCCGCTAGATCCGAAGCCCATCCCCACTGCGGCCAGCGGCCCGG
CGGGGCGCTGCGCTCGGGACCCGCACCGAGCCAGGCTCGGAGAGGCGCGC
GGCCCGCCCCGGGCGCACAGCGCAGCGGGGCGGCGGGGGAGGCCCTGGCC
GGCGTAAGGCGGGCAGGAGTCTGCGCCTTTGTTCCTGGCGGGAGGGCCCG
CGGGCGCGCGACTCACCTTGCTGCTGGGCTCCATGGCAGCGCTGCGCTGG
TGGCTCTGGCTGCGCCGGGTACGCGGGTGGCGACGGGCGTGCGAGCGGCG
CTCTCCCGCTCAGGCTCGTGCTCCGGTCCGGGGACTCCCACTGCGACTCT
GACTCCGACCCCCGTCGTTTGGTCTCCTGCTCCCTGGCGTGCTCACTCGG
GGACCCGCGACTAGCGACCGGCACGCTCGCTGTTGCTACCTCTTGCCTCT
TGCCAGAGAGCGCGCGGACCGTAGCGTTTATAGGACCCCGGCCATTGGCT
GGCGACGCCGGTGTGTCAGGGGTGTGTGGGCAGGACCTCGGGGCGGGGCC
TGGGGCCCAGCCTGTCCTGGGCGGCGCTCTGCGGGGAGGGGGTGAGGGGA
GTGGCCGGGGCCGGGGCCCCCCCTAACCACTGCCTCGCTCAGGCTGCCGA
TCGGCTCGTTCTCTCTGCGTTGGGACCGCGGAGGGCATTCGCTGTGTGAC
TCAGGGCAAGTGTGCGCACCTCTCTGAGCCTCCGCGGCCTTCTCTAGCAA
AATGGTGGAGCCGGTACCCGGCTGTAAGGCAAGCTGGGATGCGCGGAGTA
GCGGGTGGAAAGCTGGACTGGAGTTCTCACTCGGCCGCTGGGTGACTTCG
GTGCACTAGGGATAGTAACAGTACCACCTCGTAGGGTTGTAGAAAGACCA
AGTGAGTTAATATTTGTAAAGTGTTTAGAACAGCGTCTACTGCATGGAAA
GTAGTATTTATTTGTTAGAGAGACTCCAGGAGGTCTTGCCCAGGGATTGA
CTTCTCCCTTCCCTGTCTCCACCCCCCACATTCCCGTTTGTGAGCCAAGC
CTTGCTTTTGCACCGGCCGGGCTCTCTTGCCTGGAAATGCATACCCATCT
CAAACCTGGGCTTTAAAATCACCTCCTCTGGGACGCCTTCCTCTACTTCT
CACAGTTCAGGTGAGTCTATGGTGACCTGTAAGTGTGGAAAATATGAAAG
GCAGGCTAACTGACTGACATGATAGGACATTAAGGACATGATAGACCTCG
GGTTTGAACCAGCTCAACTGTATACTGGCCCGGCCGCGATCCTGGGCGAG
TTCCTGGCCAGTGAGGCTGTTTTCGTTTTTTTCTTTAATTTTTTTTTTTC
TCAGACAGAGTCTCGCTCTGTCGCCCAGGCTGGAGTGCAGCGGCACGATC
TCGGCTCACTGTAACCTCCGCCTCCCGGATTCAAGCAGTTCTCTGCCTCA
GCCTCCCGAGTAGCTGGGATTACAGGCGCCCGCTATCACCCCTGGCTAAT
TATTTTTATTTTTAGTAGAGACGGGGTTTCACCATTTTGGCCAGGCTGGT
CTTGAACTCTTGACCTCGTGATCCACCCACCTCGCCTCCCAAAGTACTGG
GATTACAGGCGTGAGCCACTGAGCCCGGCCGAGGCTGTCTTCTTATGAGT
AAATGGAATTCCCACACCAATCTCATTGCTATCAGGAGAGGTGCAATTTC
CAGATAAGCTCATGAAGTGTTTAGCATGGAAGTTCTCATTTACTGGTTGG
TGGTATTGTTGGTTTATTTATTTAGTCTGTTAGTAAATAAATAAATGCTG
GTCTCAAACTCCTGGGCTCAAGTGATCCTCCCACCTTAGCCTCCGTAGTA
GCTGGGAGTGCAGGCATGTGCCACTGCACTGGTAGTGGTATTGCTATCAT
CTGACTTCACCTTATTTCCTCTAGACCAGTGCCTTACTTGAGACACAGTG
AAGGGAAGGACCCCCAGTCCAAAGGTCTGATCAAAGTCTTGAGGGTCCCT
TTCCCGATGGAGGGAGTCATGGTGGGCTTACTGTCTGTCGGAAACCGGGA
GGCTGAGCCGACCAGATGACCCCTGGAGTTCTCTGGCAATGTGTCCAGTT
CTGGCTGAGGGGCACCTTGGTTGAGTATGGAGCCCTGAAACAGAAGGCAG
GTGCATTCTGGGCACCCCCCTGCCCAAGGCTTCAATTCCTGTGGTCTCTC
TATGCTCAGCCTGGAAAGCTTGGGCCACCTCTCTCCTCACCCCTGGACAG
ACCAAGTCCTGCTGACTCCGCTGTCTACACCACCTCATCCTCCTTCCCTC
CCCTGCTGTCTGCAGGGCCTCCATCTCCTCTGGTCTTTTGTCCTCCAGTC
CTGCACCCTGCCACTCTCCTGCTCATCTGTCTGAAATTGTCATGGAGGGA
GTAAGGGCCAAGCCTGGCCATCACGGTCCTTCTTCATGGCACCAGTTTAC
A
Seq. ID. No: 89 SLC2A2 promoter
ATGTCATTTGTCTGATTTCTGTGAGCATTTTGGGCATATTCAGTGTGACA
ACTCAGGTCTCTTGACCCCAGTTCCTTGCTTATCCTACTTCTTTCAAAGT
CTGGATAGATTCACAAAGCTGAAAAACATTGCACTACCTCAGTTCTCATT
CTTTCTGGGTTCCATTGTTAGGCAGTTCCTTGGTTATCAAGAAAAGATCT
ACAACGTAATTTCAAAAGCCCCTAAGAAAATTAATGATAGTTTTGAATGA
ATCTTGAACATCTGCGATGTGCCAGACACTATGTTAAGCACATTACCTTC
CTTTTTGCAGTAGCTTTCAAACTTTAGTTATTTGATTACTACCTTCATGA
ATTTTGCCAAATCTACATACCATCTATGTAGTTGTTAACTTAATGTTTGT
CTTTATATTGACCTTAAAGTTCTAACTACTTTAGCTTTATCCTGGGCTAT
AATACTCATGAAATCATGATGTGCTAATTATTCTTTAAATACATACTATA
TAAATGCTATATAAATAGCATTTTATATTTAACTTTAAATATCAAACATA
TAAATGGTGCTTATATAATATGTATTTAAAGTGTTTATACATTATAATAT
AATTTATCTTTTATAATTTATAAATACTATTTAAATATGTATTTAACACT
ATTAATAATTATATAACTATTAACATTAAAATTGTTGTCTAAGCCTCTCT
AATATAATCTCATTTAATTACTCCCTAACTCTAAAGTACTAGTATAATCC
CTGAATATCCCATATTATATCACTGAGCCCAAGTTTCTTTGCCTATAAAG
GGTATATTGTATCCTAATATGTTTTTGTTTTCTCACAATAGAGAAAACCC
AAAGCTCAGTTAAATAACTTAACCCAGGCCTCACAGCAGTTTAGTGGCAT
TGCTAGAACCCAGATCCAAGTCTGGCTGACTCCAAAGCCAGTGCAGCTAA
CCATGCCATCTAATGACATGAAGGAATGTTCAGCAGAGTAAGTTGCACCA
GATGAGAACTTAAAGGTGAGACCCTACAGGGCACAGATTGATCAACTTAT
ATGATTGTGTGTACAGGGGTGGGTGAGTTTAGAAACCACGTATAAGGGAG
CAAGGACACTATATCCTTTATATTTTTAAGTTTTTTTTTAAAGTATCATA
TCTAGAGAGAAGTTGAAACAGTAGTGAAGTGATCATTCCATAACCTTCGC
CTTTATTCACCACTGGTTAACATTCACCAATTTGTTGATCTCTCGCTCTG
TAGTGTTTGTGTGTGTGTGTTGTTTTGGCTGAACCATTTACAAATAAGCT
GTAGATATTTGTGTCATTTCACCCCTAAATACTTTAGCATGTACAAAGAA
CAAAGACATCCTCCTACATAATTAGATACCATTATTATATCCAAGAGTGT
GAAATTGATACAGTAATATTATTTAAGATGCAGCCCATATTTAAAATTTC
CCAACGTCTCCATAGTTCCCTTATTGCTATTTTTTTCTTCCTTCCACAAT
CTAAATCATTTGTTGCATTTGGTTGTCAGTTTGTCTTAATCCCTTTTAAT
CTAGAGAACAACCCTTCTACCTCTTTTGTCTCTCCTGCATTGACGTTTTT
GAAGATTCCCGATGAGTTTTCATGATTAAATTTAGGTCAATCTGCCCCCT
TTTTTTTTTTTTTTTTTTTGGTTAGGAGGGGACATATTTCTTTAGTAATA
TGGTTATGCACAATTACTTGAAAAAACTTATGCAGACTAGTCAGTTAACA
GAGCAAAAATCCTTAAAACCCCAATTACTGAATAAGAAACAGTTCTTCAA
TTGGCAAAGTAAAGAGAATTAATTTTGATCTGTGGTAGCTACACCCAACC
TCCCCCAAATCCTCTGTATGACTTGACTGTATTCCCCTAACTATCTCCTG
AAAACCAGACTTGAAATGAATATAATGCTGCTTTACCTTATCTTCTGTCA
TTGTACTAGTTGGGAGTCCTGTCAATTCCAGGTCTTGTGTGAGTGTGGCA
CATGCACCTGACTAGCTCCTGCCTGTTTATTCGCAGCTCTGCCTAGGCCT
AGTTATGCATTGCAGGAGGAGGAGCAAGGAGAGGGAGGAGTTGGGCTACC
CAGACCTGAGAGTCCAAGTCTAATCTTCTCAGCGGCAGCTGAATATTGAT
GGGAATCTAATCAATAAGTGCTTTGCCTCAGCAACCTGTGGCTTCAATAC
CTTTGTTCCACTAGGAAAGAAAATGGGAGCAGGTCCCTTAGGCATAAGTT
ACAAACCAGAGGCAATCACATTTCAAGGCATGTGTTATGTGGCAGTAATT
GGGAGCTGGTGAAAAGAGAAATCTTTGGAGTCTTTTGTAGAAGATGAGAT
ATGCTGAGCTGCCGTGCTGTGCTGTGGAGCATGGGAGGCACACTGCACTC
TGCTCCACAGATAATTAGTGCTGTGGTGTTTCTGTTTAGAGCTGAGCTGT
GTGGCCGGAGACCATTGTTGACTTGAATGGTTACCTGGATGAGCTTTGGA
TTGTTAGGGACTTGGGTTTGAATCTCAACATTTTACTGGGGAGTATAACC
TTAAGCAAGTGGCTCACTGTGACTTTTCTCTGATTAGTTTTTTATTTTTG
CCTGATAAAATAGAAATAACACACATTTTGTTTTGTTGAGTGGATTAAAT
GAGATAATATGTGAAAGTGTTTGGCATATCTCAACATATAATAAGTACTT
AGCAAATAGCGGTTGTTATTATTATTGCTTCATATATGTCAAGAAGCACT
TGCTGGAGGCTTTAACGTAGAGTTCTCAAATAAACCTGCTCATATGGCTT
GATCTTGGGCCTAAGTATCCCTTATGATATCCAAACCCTGTGGGGGTTCA
GGAGATATCAAATTTATTGAGCTTTAAGTGTGTGTGGCAACTGGGGCCAC
AGATTGCACTGAGGCTCAAATGGAACCAGTTCCCAGCGGAACAAAAATGT
AACAAGCAGCAACACAATGAGGGGTGTTATGAAGAAACAGGGTGAGAAAA
TGAAGGATTAGAGGAGATTCAGATTTCACCTTTTTATTTTGCCACCCTGT
TTTCATTTCTGTCACCTCTGCAAGGTGACACACCTGAAATGTTTGGTTTG
AGAAGGTAATTCAATCAAACTACAAACTTTTACTGAGTCCAGCACAGTTC
CATGCTCCGAGATGACTGAGATAAATAAGTCACAGCCTCCTCTTTCTAGG
TGTTTATAACATATAAGATGAGACATATGTAATGCAGACACAATACAATA
ATTTCATAGTGGAAAAGCGTGAGCAAATATTAATAGTAATGATGGTTAAT
CTTTACTGAGGTCTTGCCGGGTGCCAGGTGCTGCTCTCAGCACTTGACAT
GGATTAACTCGTTTAGTCATAACATGAGGTGCTCTGTGTCATAGTGCCAA
GCTTTGGGGAGCAGCTTTGAAGTCAGACTTTAAAGCGCCCAGTTAGCCAC
GCTGACTTTCCGCATCAGTTTTGGCTGGTGATGAGCACGCCCCATTGCCA
GAGCGGTCCACAGTCAGGAGCGGTTCCATGGCATTTGCCAGTGACATGAC
ATTCTGCAGGAAAGCGTCTCTGTGCCGCGTCGTAAATGATACCGGTGTGG
TTTACCAACCTTTGTCTTTTGAACCTGCCTTTTCAGAAACGGAATTTGCC
TTGAGTGCGCGGGCGGCCAGTGAGCCTTTAACTTCTTGTGCTCAGCCCCG
GCCTGCCGGCGCGGAGCACGGCGGCAGGAAACCATTCTCTTCTCCCATTG
GGAGCGCGACCCGCCATTCACTAGCGGGCCTTCGGGGCACCCTGATCAGT
TTCCTCAGGTTCAGCCAAGGCCTCTGTGGTTCTGCTCTTTCTCAGAAATG
AATGAGTAATCTCTGCACCATCTGGGATATGCTAAAACTTTATCTCCGTG
CGGGGAGGGGGTGCGATTCATTTACATTGATGCTCTGATTGCTTTTGCCC
GCGGAGAACTGATAAGCGATTCCAGCCTTTCAGGAGAGCGTGTCTTGGCG
C
Seq. ID. No: 90 SLC2A3 promoter
TTGCATCAGGCAAATGTAGGCATAGAATTCCACTCACGAACTGGGAGGG
C
AGAGAAAAAGACAGTCTCGGGTAGGATTACTAAGAATTGACCATTCTGGA
AACAAATACAGTGCAGTCTGATCTGTACCCCAGACTCCACGGAAGCCCAC
TGGTGGTGGTGGAGGGGGTGGTAGTTTGTGTCTTCACTGCTGGGTGGCTC
TTTGAGTCTCTTATCTCTCAGGACCATGTAACCTACTGGATCTACTCACT
GCATCCTGGCCCCATTTCAGTCTTCGTTTATTAAATACAGTTCTGCTATC
CCTAACTCCCGCGCAGGGGAAGAAAGTACTTACTCTGAACCCAACATATA
AGGGCGAACCCAACATATAAGAGACATTTCTTAGTGGGGAGAACCGGTCA
TCTTCACCTTGCCTGGAATAATTCAGCCGGGCGAGACATTTTCCATTTCT
ACCTTGGGGCAGCTCAATTTTTATACCTGAGGCCAACTTCTACGCCATTC
CCCCCCACCCCACCACCCAAAATCCCTTCAACCTTTCATATCTTAAACTC
CTTTCCTTTCAGGTCGGGCAGATTAGGCAGCAGGCTGGGAGCTTCAGCAG
GAGAAGTGACCTTGCCTGCAGCCTCCCAGATGTCTCAGTCCCAGCTAAGC
ATCGGTACCCCGGAGGGCCCTGCCAGTGCAATGCTCTGTTAAATCTTTTT
CCGTTTAGTAAAAATCTCTTCTGTCTCCTCTCATCATTCACTATCTTTGA
GAGCCTTTTCTTCTTTAGTCTCATAATTTTCCATCCCTTTCTTATTTTCC
TACATTTTCTTTCTTTCTGCTTTGTAGAGATGGGATCTCACTATATTGCC
CAAGGCTAGTCTCAAACTCCAGGCCTCATGTGATCCTCCTGCCTTGGTTT
CCCAAATGCTGGGGTTATAGGTGTGACCCACCACACCTGGCCCCTTATTT
TTAGTTAAATTTATCTTGGTAAAAAATGCCTTTCTTGCTGCGCGCAGTGG
CTCATGCCTGTAATCTCAGCACCTTGGGAGGCCGAGGCCGGCGGATCGCC
TGAGGTCGGGAGTCGAGACCAGCCTGACCAACATAGAGAAACCCCGTCTC
TACTAAAAATACAAAATTAGCCGGGCCCGGTGGTTCATGCCTGTAATCTC
AGCTACTCAGGAGGCTGAGGCAGGAGAATCGCTTGAACCCGGGAGGCGGA
GGCTGTGGTGAGCTGCGGTCGCTCCATTGCACTCCAGCCTGGGCAACAAG
AGTAAAACTCAATCAGAAAAAAAAGAAAAGCCTTTCTCCCTGCAAATAAT
GCATATACAACACTCAATATTTTAGTATGTCCTGCACTCACGATTTTAAC
CCTAATAGGAAGGACAACTGGAATTCTTTCTCCTTAGCATCCACGAACCC
CATATGTTATAGGACAAACAGATGGCCAGACCCTGGCCTGTGCCTCCAAA
CACAGACCTTTTCAAGGTCTAATGCTTCCAGAGAAGTCTTCACACTGGCT
CTGAGACCCACACTTGTAATCCATACAACCAAGCAGCCCGATCCTCTAAA
CACTCAACCATCTCGTCCTCAGTGGCATATGGTTACCTAAAATAAACCTG
AGAAGCAAACACAACTTCTCCGGGTGACTTTCCTATGATAAGGTGAACAG
AACTGTATGACATTCAAAGAGAACTGATGAAGCGGCAGCAGTGCAGGCTA
GAGACGAAATGAAAAGGCCTTAAGATAGCTTGGTGACTTAGGAGAAAATA
GAACAAGGAGATTACCATTAATATTTGCCTTTCTGAGTGTATCCCAAACA
AGCAAAAATAACCAGTAATTATATCATTCTTACCTTCTGTGTCCCCATCG
CTGTAATCTAATTCAAGTCTTCAAGAAAGATCTAGGGGTGATTCCAGAAA
CAGCTTTTTCAGCCAACAAAACCTTCAAAATGTCCTTCTCAGCAGCAAGT
TTTCTCCACGTCCTCAGGAAGGATCCAAAGTCTTACCATTACAGCATCTC
TGGGTCTCGCTGGGATCATGACTATTTATAATCTCCGCAAAGGGTGGAGC
CTGAAAGGGCGACAAGCCCCCAGCCCCACCCCACCCCACCCCACCCCCCT
GAAGCAATCTTGTGATCTCTCGGAATTCTGTGTTATCAGAACTACCCCCG
CCCCCGCCCCACAGACAATAACGCCCTCCCTCCCCCCTCTCTTAAAACCA
CATCCTTCCACTCTCTCCACCCTGGGTTTTTTTTTTTTTTTTTTTTTCAA
AAGCTCATCCTTACTCCTTCCCTCTTCTAATAGCAGCTAGAGCCTCTTAG
TTTCACAGACAAAATACTACCAGAAAAGCCTGGAGGGAGGGCAAATACAC
TATTTTATGCTCTCGCATCTGCTTGGGATTAAAACAATCTACAGAGAGAT
TAGAATAAATCTATCCATACAAAACGAAGTCAAGTGGGTGCTACTATCAG
AGATTTAAGGTTGTTAGAAAGAGTGTTAGTAAGAATTCTTGATCCTATGA
ATTTATAGGCTGGGTCTACAATCTTTTTTTTTTCTTTTTTTTTTGAGACG
GAGTACACAATCTTAAAGGGGAGGGAAAGACAGCCTGAGAGAAGAAAATC
AGGGAAATAATTTTGCCAGTGTTCCTTTCTTCGCATAATCTCACCTGGAG
GGAAGAAAATCTGAATTTCTAGGAGCCAGACCAACTATCAGGGCAGTAGG
AATAAGAGGTTCAACTGTTTATGGAATACAATCCATATCCTTTTTTTTTT
TTTTTTTTTTTTTTTGATATGGAGTCTCTCTCTGTCGCCCAGGCTGGAGT
GCAGTGGTGCGATCTCGGCTCACTGCAACCTCCGCCTCCCGGGTTCAAGC
AATTCTCCTGCCTCAGCCTCCCGAGTAGCTGGGACTATAGGCGCCCGCCA
CCACGCCCCGCTAATTTTTGTATTTTTAGTAAAGACGGGGTTTCACCATA
TTGGCCAGGCTGGTCTCAAACTCCTGACCTTGTGATCTGCCCGCCTTGGC
CTCCCAAAGTGCTGGGATTACAAGTGTGAGCCACCGCGCCGAGCCCTATA
TTTAATAAGGCATTTGCCATATCACCCATTCTGAATTCTGCATCACTTTC
TGCTAGTCTCAAGCTTATAGCATCTAGAAATGCAATAAATACCGAATTCA
ATTAAGCAGGTATTTGCCGAATACACGCTATGTATCAGGTACTCTCCAAG
AAGCTGGTGATTCTTCAGTGAATAATAGAAAAAAAGGCCGGGCGCGGTGG
CTCACGCCTGTAATCCCAGCACTTTGGGAGGCCGAGGTGGGCGGATCACG
AGGTCAGGAGATCAAGACCATCCTGGCTAACACGGTGAAACCATTCTCTA
CTAAAAATACAAAAAAAAAAAAAAAAAAAAAAAAAATTAGCCGGGCATGG
TGGCGGGCGCCTGCAGTCCCAGCTACTCGGGAGGCTGAGACAGGAGAATG
GCGTGAACGCGGCAGGCGGAGCTTGCAGTGAGCCAAGACAGCGCCACTGC
ACTCCAGCCTGGGCGACAGAGCAAGAATCCGTCTCAAAAAAAAAAAAAAA
AAAGAATAATAGGACAAAAGAGTTCTGACCCACAAGTCATTTATAGTGTT
TCCCTTCTTCCCTTTTTATAAAGTATAAAAAGGGACTAGGGCAAGTAATT
CTCACATAACTGGAAAAGATTATTAATATCCTATTAGTTATGGACTGTGA
GTTTCTTGAAAGAAAAGTGTTACATAAGTATCTGATTACTAAGCTATACA
AGAAAACTGAACAAGCTAAGCAGGAGAAGAAAATAGGAATGAGAACTGAG
GGAGGGATAATCGGGGAGAGAGAAGAGTGGTTCCAGGTTGAGAGTAAACA
AAGCAAAACAACTTGGTTTGTGTTTAAACAGCTGGGTAAGTGAGAGATAG
TATCTAGGCTTTTTTGGAGGAGGAATATCAACTATCAAATTCCTAATGCC
CATTCATTTGTAGCTTGCAACCCTTTCAGTACTGGAGGTGGCAGGGTGGG
T
Seq. ID. No: 91 SLC2A4 promoter
CCGGATTTTTTTTTTTCTTCTTGAGACGGAGTCACTCTGTCGCCAGGCTG
GAGTGCAAGGGCACGATCTTGGCTCACTACAACCTCCACCTCCTGGGTTC
AAGCCATTTTCCTGCCTCAGCCTCCCGAGTAGCTGGGATTACAGGTGTGC
ATAACCACGCCCGGCTAATTTTTGTATCTTTAGCAGACATGGGGTTTCTC
TATGTTGGCCAGGCTGGTTTCAAACTCCTGACCTCAGTCGATCCACCTGC
CTTGGCCTCCTAAAGTGCTGGGATTACAGGCATGAGCCACCAGGCCGGGC
CGGCATTCCAGATTTTTCAGGGGATTAGTGCAGCAAAGGAATCAAGAAGG
GATGTAAAGGCACAGTGTGTTCTGGGTACAATAAGGACTTAGGCATTGCC
CAGAACAGGAGGCGAAGGAGATAGAAGGAGAGGCAGGAGAGATAGGTAAG
GCCAGAGATCGGATAAGAGAGGCAGGAGGTTTTGTTCACTCTGAAAAGGG
ATTTGAACTTGGCAATTGGGGCAACAGAGACAGTGACTTCTTGCTTGAGA
GATGAGATTGGACCTTCGAAAATTGTTCTCTGCCCTCGTCATAAAGGAAA
TAAGAGGAGCACGAAGACCAGTGAGGGTGATGGTGATCTGGACTGAAGTG
GCAGCCGCCACGGAGAATATCGGATGAATGTGAGAGAGTTTTGGAGGTCA
AAGCACCAATGTTGGAAACTAACTGGATAAACGAGGAGAGCGGCGCAGGA
CAGGAGGAATCGAGCCTGACTTCTACCATAGGGGTGACTGGGCGGGTAAT
TCATTGAAATAAGGAAGTTAGGAGGAGGAGCAGGTTTGGACATGCTGATC
ACTAGAGCTGCCACATCCGGGCGGTAACGAACACCTGGATCTGCAGCTCC
AGAGAAGGGCCTGGGTCAGATGTCACTGAAGCCCTATGGTGGCGGAAAGG
CGAGAAATAGTGGGTTGAGATTCCAAGTGCAATCCACTGCGGCTCCTCGC
TCGCCCTCCAGGTGGCAGCACAACCCTGCGCTTCCGAAGCCCGTTTTCTG
AGCCAGACACTCTCCACGCTCTGGGTATTTCGGCTTCTCTCTCCCCACAC
GCCGACCCTAGGTCGCGCACTTTCTGCCTGGCAGAATTTGGCCGAGGATC
CAAACCCGGAGCAGCCTCCAGAGAGCGTGTCGTTCACGCGGCCAGCATAT
GCTCAGAGACCTCAGAGGCTCAGAGACCTCAGGGCTGGTGGTGTGGTCGG
TTGTGACCACTTGTCCCTCGGACCGGCTCCAGGAACCAACCTGGGGAATG
TGTGTAGGGGAAGGGCGGGATAGACAGTGCCCGGAGCAGGGAGGCGCTGA
AAGACAGGACCAAGCAGCCCGGCCACCAGACCCGTTGTGGGAACGGAATT
TCCTGGCCCCCAGGGCCACACTCGCGTGGGAAGCATGTCGCGGACTCTTT
AAGGCGTCATCTCCCTGTCTCTCCGCCCCCGCCTGGGACAGGCCGGGACG
CCCGGGACCTGACATTTGGAGGCTCCCAACGTGGGAGCTAAAAATAGCAG
CCCCGGGTTACTTTGGGGCATTGCTCCTCTCCCAACCCGCGCGCCGGCTC
GCGAGCCGTCTCAGGCCGCTGGAGTTTCCCCGGGGCAAGTACACCTGGCC
CGTCCTCTCCTCTCAGACCCCACTGTCCAGACCCGCAGAGTTTAAGATGC
TTCTGCAGCCCGGGATCCTAGCTGGTGGGCGGAGTCCTAACACGTGGGTG
GGCGGGGCCTTTTGTTCCAGGGACTCTTTTCTCAAAACTTCCCAGTCGGA
GGCTGGCGGGAACCCGAGAGGCGTGTCTCGCCAGCCACGCGGAGGGGCGT
GGCCTCATTGGCCCGCCCCACCAACTCCAGCCAAACTCTAAACCCCAGGC
GGAGGGGGCGTGGCCTTCTGGGGTGTGCGGGCTCCTGGCCAATGGGTGCT
GTGAAGGGCGTGGCCCGCGGGGGCAGGAGCGAGGTGGCGGGGGCTTCTCG
CGTCTTTTCCCCCAGCCCCGCTCCACCAGATCCGCGGGAGCCCCACTGCT
CTCCGGGTCCTTGGCTTGTGGCTGTGGGTCCCATCGGGCCCGCCCTCGCA
CGTCACTCCGGGACCCCCGCGGCCTCCGCAGGTTCTGCGCTCCAGGCCGG
AGTCAGAGACTCCAGGATCGGTTCTTTCATCTTCGCCGCCCCTGCGCGTC
CAGCTCTTCTAAGACGAGATGCCGTCGGGCTTCCAACAGATAGGCTCCGA
AGTAGGATTCATCATGAGGGGGCGGGGGGGGGGGGCACGGGTCCCGCTTT
TCTTGGGCTGGGGTCGCGGTTGGGGTCAGCTGGGGGTGGTTCCTGCGCAG
GCGCAGGGGGTGAAGGTAGGGGGCTGGCTATTTATACCCGGCCTGGACAA
CCCGTGACTGTGAGATTCCAATCCTACCAAAAGGCAGAGTGGGTCTGGAG
GGCCTTTCGGGGCACAGGCAGCAAGTGGATTCTGCGAGCCAGGGTTCACC
CCCTTGCCTAGTAGGCGGCGCGGCGCTCCGGAATCGGGGACACCCTGCCC
TCGATCCGACTCGGGAAAGCAGATCCAGGCGGGTCTTGCCCTCCGGGAGC
TGTCCGTCCGTCTTCGCTCACGGGCAGTGTTTCGAGGACCGGAGGCTCTC
CGTGGGCCCCCACCCCCACTCCTGGCCGCCCTCCAGGACGCTGAACTTTC
CCTTGGCCCCATGGTTGGTGGAGGGGCAGAGGGGACTGTCAGCCCCCCCT
CCTCCAGCTCAGGTTTCCGCTTGGAGACAGTCTGTGCCGCCAGCGAGCGG
CCACCACTGCCACCGCCCCTCACACTACCTTCCTGCCCTCCTCCCCTGGG
CATGGCTCTCCCAGGCAGAACCCCTGGACGGCCCTCCCTGCACGGAGGTT
AGAGGGGGAGGGCAGGCCACGACGTAGATAGAGAAGGCCACCCCTAGATG
ACCGGGATGTCCTTTCTGGAACAGCACTTCTTGGTCCTGTTGGGGGCCTC
CTGGAGCTGGCTGACAGAACCCCCAGAGGGGAGGGAAGAGGACAGTGGCT
GATGATAATAATGCACGTGTTAATTTATGAAACCAGCACTGTCAAGGATA
TTGTTAACATGTGATGTTGATTTTCACAACACTCTCACAGATAGGTAGGC
AAGGCAGGCAACATCACCCCCATCTCACAGAGGACACTCAGGTTCGGGGC
AGGGAAGTGACTTGGCCAAGGTCACACAAATCTGAGCTCTTAAGGCCAAG
CCTGTCTCAAGGTCACAGAAGAATTTTGAGACAAGTTCTCAAACATTTCT
CTGCCTTATGGACCCAAACATCCAGTTTCTCCTTTATGCCCAGGTTGCAG
TTCAGCTCCTGTTTACATTGAGATCTTTGTGCAATTCCTAATATGGCCCA
GTTTCCCTCACCCAACATGTTGGGTGGAGCCCAGTATCTTCAGGCTCCAG
CTGGGCCCGGGCCCCTAGCGGAAGGAAAAAAATCATGGTTCCATGTGACA
TGCTGTGTCTTTGTGTCTGCCTGTTCAGGATGGGGAACCCCCTCAGCAGC
GAGTGACTGGGACCCTGGTCCTTGCTGTGTTCTCTGCGGTGCTTGGCTCC
CTGCAGTTTGGGTACAACATTGGGGTCATCAATGCCCCTCAGAAGGTGAG
GGCCTGCAGCTGGCAGGGTGGGGGTACCCAAACGAGGAGGACAGGTGTCT
CGGGGGTGGTGGAAAGGGGACGGTCTGCAGGAAATCTGTCCTCTGCTGTC
CCCCAGGTGATTGAACAGAGCTACAATGAGACGTGGCTGGGGAGGCAGGG
GCCTGAGGGACCCAGCTCCATCCCTCCAGGCACCCTCACCACCCTCTGGG
CCCTCTCCGTGGCCATCTTTTCCGTGGGCGGCATGATTTCCTCCTTCCTC
ATTGGTATCATCTCTCAGTGGCTTGGAAGGTTCGCAGCTGGAGGGCAGGG
GTGGGGGAAACAGGAAGGGAGCCACTGCTGGGTGCCCTCACCCTCACAGC
C
Seq. ID. No: 92 MLXIPL promoter
GAGACAGGTTCTCAGTATGTTGCCCAGGCTGGTCTCCAACTCCTGGGCTC
AAGCAATCCTTGGATGAGAACCTTGGCCTCCTAAAGTGCTGGGCTTACAG
GCAGGAGCCACTGTGCCCAGCCAGAGATTTACTATTAATATTTGGGTATT
TGGGAAGAAAACACAGTCCTCTCTACCCCAGCTATCTCTGACTGGGGTTA
CATCAGCCTTGAGTCAGCCTCTGGGTGGGGGGGCAATTTTGGGGGTGCGC
TCTGTCCCTTACCTTGCCAGCTCCCCAGAACCTCTCCTCTTTGGTCACCC
ATCGTCAGGGTTGGAAGTGAGGCCCCCTCCCAAATAGAGTGATGTGTAGT
ATATAATGAGCAGCCAAAGTTGGGGTTGGGTGGGAAATGGGAGACCCTTA
GGAGGAGGAGTGCCGGCATCCGGTGTACCAGGACCTTAGATGCCCTGTCC
CAGGCAGTCGCGTGACATTTGGGGAGGGAGGGAGAAGGGACGGGAATTTG
AACTTTCCACACGGGTCCGACGCCCTGGCCGATCGGGTTGCAACATGACC
TGGGCCAGGGGCCAGAGCTTGTGGGCCAAGGTTACTCCGCCTCCAGCCAA
TGGGGTCGCAGCTGGGCGGCTGCGGTTGGGGGCAGGCAACACTCCCCTCA
CAGTCCCCACCCCAGCTCCTGCTCCCCCGCAGGGCGGCAGGTGCCGATCT
GTAGCCAGCGGTTCTCAGAAGGGGAGGAGGCGCCGCGGAGGAAGAGGAAT
ATTTGCACAGAGAAAAGATCAAGGCCGGCCGATCCCCCTTTCTCCCCTAC
GGGTTTTCGGTTGATAATTTATACTGGCCCCTCAACTCCGGGGAACTTTG
CTCCAGGGCACTGGGCCAGGCCTCGGGTTCGCGCCCTCCTCTGGGACGCG
GGGCGTGGAGCGGCAGCGGGGCGCGGCCTGTGCGCTCTCGGTGGCACTAA
GCGGGGGTCCCAGGCAGCGACGGGCGCTCAGCCTAGTGTGGCGACGTGGG
TAGGCGAACCCAGGCCTCCCGGGCACCGGCGTAACCTCTGCGAGCTGCGC
CTCTTGGACATTTCTGGGACATCCTCCTCCCGCCACCCTTAATGTAGCAG
ATCAAGAAGAGGTGTCTCAGCAATTGCGGGGAGGTGGACGTGAGAGTGGG
CCAGTTCCAGTCCACTTTGGTTATGAACTGGGACAATCAGGGGCGCTGGG
AGTATCTCAGGGTCCAGGAATAGGGCGGGCTTGGGCCGGGGCTGCTGGGG
AGAAGGCCGAGGCCTGCAGGGGGTCGGGTGGAGTGGCTCCAGAGTGGAGA
GTGGGGATGGGCGCGGGTGGCCTAGGGACCAGGGGCGCCTGGCATTTTTG
TAGGGACGGAGGGGCTGGGACCAGAGAGAGGGGACCAGAGGTCCAGGTAG
AGGTGGGGCGCAGGCGGTCTGCGCTTCGGGGAAGAGGGAATGGGGGAGGA
ATGGGAATCAGAAATGCGTGGGTCCGTCAGGGGCCAGCGGGCTGGGCCGC
TGTAGGAGGACCTCGGGGCTGGGAGAAAGGGGGTGTCCAGGGCGTGATCG
GAGCCTCCGGGAGCCGCAGGACCGGGCTCGGGTGGGGTGTTGAGGGCCCC
AGCGCGCAGTCCTGCCCCGGACCCCCCCCCCATCCCCACCTGGAAGCCAA
GGCCGTCAGGGCCCGGAACCGCCCTCACCTGTAGGCCAGGCTCAAGCACT
CGAAGAGGCGTGTGAGTGTGGGGTCGATACTGCGCGGCCCGAAGTCGGAG
GGCCCCACGGACCCCTCCTGGTCGCGCCGCCGGGGCAGCGAGTCGCTGTG
CGGCGACGACACCATGAAGTGACCGCTGTGGATGACCTGCGAGCGGAGCA
AGCCGCCCGCGCTGCGCCGGAGACTCGGGTCCTCCGAGTCTGTGTCCGAG
TCCGAGTCTGGGCTGGGCGCGACCCGCGGGACCTGCAAGCCCGCGGCCAG
ACCTGCCAGCGCGCCGGCCATGGCTGTCGCCGCCGCAACCGCCTGGTCCC
TGCTCCGCGCAGCGCGGGGAACAGCTCTTGGCCAGCCGGGCCTCATTAAC
ATAGCCCCGCCCCCACACCATAGGCCGATCGGGTGGGCGGGGCCTGGGAC
GGGGCGGGGCTTGTATTAGCATAATCCTTACGCCAGGTGAGAACCCGGTG
CTCTGGAGCTCTGGCGGGTTGGCCCCATCTCCCTTCCCGTCTTTCCTTGG
AGTCCTGATAGAGCCGCCCCTACTTTGCTCCAAAGGGATATTAAGAAAGG
TCAGGCCGGACGCCAGGGGCTCTTGCCTGTAATCCTAGCACTTTAGGAGG
CCAAGGCGGGAGGATGGCTTGAGGTCAGGAGTTCGAGACCAGCCTGAGCA
ATATGGCAAGACCCCCATCTCTACCAAAAAAATGCAAAAATTAGCCGGAC
ATAGTGGAGAAAGCCTGTAATACCAGCTACTGGGGAGGCTGAGGTGGGAG
GATTCCTTGAGCCCAGGAGTTCGACAATATGGCAAAACCCTGTCTCTACC
AAACATACAAAAATTATCCAGGCATGGTGGCGTGTGCATGTAGTCCTAGC
TACTCCGAAGGCTGAAGTGGGAGAATCGCTTGAGACCAAGAGGTTGAGGC
TGCAGTGAGCGGAGATCGCGCCACTGCACTCCAGCCTGGATGACAGAGTG
AGACCCTGTCTCCACAACAAAAGGGTCCCCTATCACACCTGAGCCTGAAA
GCGCCTCAGGACCTGGGGTTTGTTCTTTCAGGGGCACCCTCCTGTGAGGC
CAGAGATCACCATCTCCCCTCACCTAACTCCTGCCCTTCACCTCCAACCC
CCGCCCCCCATATTAGCTCTTCTGGGTCCTCCACATAGGTAGAAGCCCCC
AACTCTCAGGGGCCTGATGTGGTAAGCCCAAGGTAATGCCCCTGAAGGGC
CTGGCCCACAGGAGGTTGCTAACACCTCTAGTTAACATGGTGGCCAACAC
GGTGGCCGGGCGCGGTGGCTCTCGCCTTCAATCCTAGCACTTTGGGAGGC
CATGGTGGGTGGGTCACTTGAGGCCAGGAGTTCGAAACCAGCCTGGCTAA
CATGGAGAAACCCCTTCTCTACTAAAAATACAAAAATTACCGGATGTGAC
GATGCGTGCTTGGCTTATAATCCCAGCTACTCGGGAGGCTGAGGTGGAAG
AATCACATGAGCCCAAGAGGTGGAGGTTGCAGTGAGCCGAGATCGCGCCC
TACTGCACTCCACCCTGGGCAACAGTGAAATCCTGTTTCAAAAACAAAAT
AAAATACTTAAGAAAAAATATATATTAAAAAAATTGTGATCATTACTTCA
CCTGCACAGAGAGGCCCAAGGGGAGCCTCGAGGGACTGTTACTGGTAGGG
ATGGGTGGCTCACTGGAAGGTGGACATCCCCTAGGCAGGCATCTACCCAG
GAGCTCTGAGCCCCTTCTCTCACTGACACTGAGGTCCAGCTTTTGTTCCT
GCTTCTTTGTATTTTTATTTATCTATATACTTCTATTTATTTATTGTTTT
GAGACGGAGGCTCACTCTGTCACCCAGGCTGGAGTGCAATGGCACGATCT
CGGCTCACTGCAACCTCTGCCTCCCTGGTTCAAGCGATTCTCCTGCCTTA
GCCTCCTGAGTAGCCGGGATTACAAGTGCCCGCCACCATGCCCAGCTAAT
TTTTGTATTTTTAGTAGAGACGAGGTTTCTCCATGCTGGCCAGGCTGGTC
TCAAACTCCTGACCTTAAAGCGATTTGCCCACCTCAGCCTCTCAAAGTGC
TGGGATTCCAGGCATGCGCCACCGTGCCCAGCCTATTTATATATTTTTAG
AGACAGGGTCTCGCTCTGTCACCCAGGCTGGAGTGCAGAGGCACGGTCAT
AGCTCACTGCAGCCTCAAACTCCTGGGCCAAAGCGATCCTCCCACCTCAG
CCTCCCTAGTAGCTGGGACTACAGGCTCATGCCACCAAACCTGGCTAATT
TTTTTTTTTTTGTAGAGATGAAATCTGCTTCAGCCTCCCAAAGTTTTGGG
A
Seq. ID. No: 93 CD36 promoter 1
CATAAAGCAAGGTATATGCTCACATAAAACACATCCCTTCAAAATATGG
T
GGGTGCATAGTCTTTAAAAAAATGAAAACCCCAACACTCTTTACTTTAAA
ATGGGTACAGCATTTTGCTATGTTATTGTTTAAGGTCTATATTTTTCTAA
AAATCCACAGTTGCAGATTTGACTCAGCTGTGTGCTTTCAGATCTTGTAT
GCTAAGCTACTCAGGAACTGTTGTCTATGGGATTGAACACTTAACAGCTG
TCTCTGGGAATGTAAATTTGATTCTTTGGACTGCTCATGGGACTCTGTGA
CATTTTCCTCTCCAGTGACAGCAGATTTGAATGGCATTAGGCTACTGATT
AAATAATACCCAAGTAAGTTTAATGTCAACTTAGGCATCTATTTTGCATG
TATTTTTTCTGAAATAATCTTTCTCTAATTGCGATCATATTTTCATTATA
CTTTAGTCTTTTAATATCTTTAAAATATGCTCTTTTAAACAGGTCTTATC
TGAACCCTTTCGTTGGTAATCTTTTTCTGAATTGTCATATTAGAGTGTGA
GTGTGTGAGAATTAAGGTTGAGAAATAAATTAGCTTAAATACAAAACAGC
TATCTACAAAATAGATATTCTTAACCAAAATAATATGTCTTTATACTACC
ATTTTATATCGTGCCACCAAGATAAAAGGTCGCAGTTATTTAAGGGAATT
ACCTTCTTAAGTTTCATGAAATCACTGTGAGTTATTCTTTTCTAACTGAA
AGCATAAATTATAAAGAGATTGCACATGGTCATTTCTACTTTATCCTAAT
TAAGTGGTCTGAGATTTGAAAAAAAAGTAATAGACTTACAATATTTTTAT
GACTTATTTTTTAAATAAATATGTCAGTAAAAACTTCCCTGGGGAAAAAA
AATTCTTGAGGAGAAAAGAGTGTTTGGGGTTTTTTTTTTCAGTTGTATGG
CACTGCTTGCAAATGAAAGGATTCGTTGTGTGCCTCACATGCCCAAAAAG
GACAGCACGAGCATTCTAACTTAAGGACTGGTTTAAATATGACCTAATGA
ACTAAATATATTTCTGTCTGGTATTCTTAGGTTACTTTACTTCTAAGGTT
GTTTGAATGCATTTATCCAGCTCTCCTATCAAATACATGCCAGGTTAACC
CTGTGAAGGTTTTCCTGTATGATTCTGAGAATTTCTTTATAAGAACAGGG
TCTTGATAGTGAAGGTTCTTTCCCCAAAGTGCAAGGCCTTCTATTTTGAT
TCAATATCAACATAATGCTTTTTTTTTTTTTGAAAGTGACTTAAGTACTA
AAGATTATCCTCAATAGTAATTCTACTTAGACATATTTTGGTTGAAGAAA
TTTAAAGAGTTTATTTTTTGTAGGAGCACTGTGCTGTGATCATGAAATTT
TTGCAAAACTAACAGAAAATGATAAACAGTTAAGAACTTCCAGCTAGAAC
AATAGGCTGTTTCCTTTTTTTAAGTCAGTTTGTCATCCTTGCTGATTGAC
AGTCTCCAATTGTTTTCAGAATTATTAGTCTCACAGTATTTCAGAATTAC
GCTGTATAAAAAAGTACATTAAATTTAAACTTAATAAAATAAGTTTCGCA
AGCTCAGTCAAGACAGGGAAGTGAGAATATGATTATTCTTCCAAATTTAC
TATTTCCACCAGCGGTCTCAGTTCAGCATCTGAGAATATGTTAGGGGAAA
CTCAGCAAGTCAGTTCCTTTTGTTTTTCTGGCCTCTGACTTACTTGGATG
GGAAATAGCCAAAAAAAAAAAAAATGCTGAATATCTCAGATATAGGTAAT
GGGTCTTCACCAGAACATAAAAATAGACCCTTATTAGCCATATCAGTAAT
GTGCTGTGTGGGGGATTTTTTTTTTCTTTCAATTCCTCTGGCAACAAACC
ACACACTGGGATCTGACACTGTAGAGTGCTTTCTCTTCTCTTTTTTTGGG
GGGGGGAGGGGGTGTGGTTGCATATTTAAACTCTCACGCATTTATGTACT
GAGGACTGCAGTGTAGGACTTTCCTGCAGAATACCATTTGATCCTATTAA
GAATTGTCCAAATGTTGGAGCATTTGATTGAAAAATCCTTCTTAGCCATT
TTAAAGGTAAGTTGTATGATTTTTCTTTAAATAAAAAAGATTAAGGGATT
TTTCCCATCACCCAGATGACGGGAGGTGACACTACTGTATAAATACTCCT
AAGAAGTTATATAGGAGGACAGGAAAAAAGGTAGTAACTGTTTTCTTAGT
CACTAGGCTTTATCTAAAAATCAGGTTTGCATTATTTTCAAGTTATCAAC
CTTCCTATATTTTATGCTTAAACCATACCCAATGGCATTTCATGTCGTAT
GTATAAGTTAATATTTAAAGTGATTTATAATCCAATTTGGATTATTATAA
AACTTTCATTTTATACTTAGGCTATTTATACATGATGTTTAAATGATGAA
GATCATTATACTGCAGACCAATATAATGAGGTTAGATGTTATAAATGACT
TACAGCTCTTATGTCAACATGTTGTCTAATATGCAATTATTAACTATTAC
TTTACAGAGTCATTAGCTTGTTTAATTTTCAATCTGAATTGCACAACTTT
TGGTATACCTGACTTGCTCTACATGATCAAATGCAATGTGTATTTTGGAT
ATGTGCTTTTTAAGTTCTTTTCTGTAATGAACAAAGATATTGTCATTGTT
ACTGGGAATAGCAAAGTATATTACTTTTAAATAATCTGAAATTTAATTAG
TGTAAATTAGTAGAATAAAAATAAAGTTAATTTTTTTTCGATTCAGGAAG
CTTATTACAAATAGTACTGAATTATTAGATGATGATGCCAAGTTGAAGTA
AAGGAGAAATCAATTTGGAGGAATTAAGTAAAAGCCTCAAATTAATTATA
CATTAACTAAAATTATAAATAACTGTAAATGGCATAAATTCTCTAGAGTA
CATCCCAGTTTCTGAAATCATCAAGTAGCATATTGAAACCTGTTTTGTAA
AGATTTGCATGTACTTTTTAGTCTTTTTCTGTGTGGTTTGAAGTCAGTAA
TGTGTGTTGAAGCTAAAAAATATTGCTTTAAATATTGGTTCACAAGGAAG
CCAAAATGAAATATTTAAAAATGTTTGTTATATAGAAAGTCCAGTGGTCC
TTTGGAATTTACAAGAAAATAAGAACTAAAGTAGAAATCATTTAATTTCC
CAGACTCATTAGTGAACCAGAACAATAAACCAAACAGAGAACCATCAGAT
AATGGGTTAGCCTGGGAAGAATATTAATTACAACTGGAGGTACTTGCACA
GAGTGTACTGAATTTGGAATTTACCTTGGAAGTTATAGTTGCCACCCTAT
TTTTTTAAGGAATTTTTTTAACCTTGTTTACTTTAACACATTTGAACCCT
GCATGTGTGTGTATTTCCTGTGTGTTTCCTGAAAAGGAAAGTTTAAGATA
ATTCTGTAACTATCAGATACTTAATTCTGTAAGTATCCTTGCATCTAATC
AGAAGATGTTTTTGTTCACTGGCCCAAGATGTGTCTGAAACTTGCAAGTA
GCATTGTTCTAAACTAGTAGCTGTCTTTCCATTTGCATATTTTAATTCCA
TACATATATGATCACATTTCATTATAATCTAACTATTTTGCAAATACTCA
TTTCTACTTTTGTAAAATATACACATTTATTAGATTTTTAAAACACTTGG
CCAATATGAAATAACACTTTACTGTTATGGTTTGAAATGATACTTCATAG
TAATATTTCAAAATAACAATAACATTTTCTACAATATAAATATATATTAT
TTTACTTCATTTAATACTTGCAACCTTATTTGATGTAAGTAAACTCATTT
TAGAGAAAACTGTATCACAGACAAACTTCAATACTAACACCCAGAACTGA
ATCTACATTTCTTGGCTTCAAGGTCCAGCATTTTTGGCATTACAAACTTT
ATAGTGTCTGTTTATAGTAGAATTTGGGGTGTGGAAAAAAGACATTAGCA
T
Seq. ID. No: 94 CD36 promoter 2
TACCACAACTTTTCCCTCTCCCTCACCACCTATCCCTATAAGCTCATGCC
CAGCAGGGAGTAAACAATTTAATGATAATGCATATATGATTCGTGGAAAG
GAAATACAGGATTTCCTGTTCTTATGGGACATAATCCATATTTGAAGATA
ACAGTGCTTACAATGTCAGAGGCAGTCTATGATATTTACTGTGTTCCTAG
AAAATATGCATAGCTGTGTTCACTGAATTTTTAACATGCCTTCTTCTTTG
ATAAAATAATTTGTTTAAAAGTTCCTAGTTCAAATAATAGAGAAACCTTT
TACACATAGGGAACAACATTTTTTTCCTTAATTAAAAAGTTCACATTCTA
CAATATTTGGAAATATGTGACCCTACTTTTCACGTAAAGACCATCTGACC
TAAAGGGCCAACTCCTTGCCACCTGATTGTCCCTTTCTCTGTCACATATC
AGACCTCTGACTGGCTGAGAGGTAGCCATCAGGGCTGGCCTTCCAGGATC
TGGAAGATCAAAATCTGCTCATACAAACTGGCAAGGAGAACGTGAAAAGC
ATAAATGCAAGAAATGAGAAATCTCCGTGGCTGGAGTGGTGGCTTATGGA
TCCTCTCTCTTTTTCCTGTCTTAGGGACATAATGCTTCTGGGAATGTCCT
GGTGTCATAGCAATACTCTTTAGGGACCATTTCTGTGCTTTCTGTAACTT
TGTTTAAGCAACAAAAAAGCAAAAAAGCTTTGGACTTGCTCAAAGGGTGG
CTAATCTGATACAGATAAAGTGTGGATGAAACTCATTTTTAGCCTGTAAC
TATGTTGTAGGATGGAAAAGAAAACTTGCTTGTGGGTCAAAATTTTGTGG
GTTAGACTCTCAAATGAATCAGTTATTCACCATGTGTCCATTGTCAAAAC
TTATCCTTTTAGTTGCAGTTTCTTCTCTGGAAAGTGAGCCTAAAATATTA
ATTTGAAAGCTTATTTTGGGATTAGAAAAGGAGAGTGAATTAGATGGTAT
CTACGTCGTAAAGGGTCAGGCACGTGAGCAGTGCTCAGTACATAGGACCT
ATTATTAAAATATGATGAAAATACTGGTTATGGTGATTTGACAAAACAAC
CTTCTTTTGTGGATAGTGGTTACTACTTGATACAGAATAGTTTCATTTAA
AGAGTGGTAACTCTAGTCTATCATTTCCCTTTAATGTGATTCTGTAAAAG
AAATTTTAATTGAAAATATTTAACTATCAGGAGAGAAATATGACTTTTCC
AGTTTCTCTGTTCACTTCGTTTTAGTATAGAATTAGAGATTCTTAACATA
TAAATCCTAGAAAGTAATTTAGAAATCTTGTCATTATATCTTCAGAGTAA
TAACTTTCATCACAGGGTCTGTTTGGGCTGAGAGTAGAGCTTTATTCATG
TTTACCAGAAGTCCCAAGCAAAAATTTTTTAAACCAAGTACATTCTACAA
CCCTTCAAAAAGATTCAAAAAACAATAATTTAAACCAAAGAACTCTGGCA
GTGCATGACATTTTCTGCCTCATTAAATGAGGCAAACAAAAAATGACCAA
ATGACTACTGAAAACATAGAGGAGAAAAAATACTAAGAAAGTTTACAGAG
ACTTTCCAGTAACAAAAGTTACTGGAACAAAAATCTGTTTTGTTGACTCA
ATAGTAATCAATATTCACTTTTATGTTGTTTTATGCTTAAGACAGGAAAA
CCTGAGCCTTCCTCTTTACTATCAGAGCAAATGGTAATCATGAAGTGAAG
CAAATTGATTATTGATTACTGATTACTTGAATTTCTATGTTTTATAAGTA
GAGCAACCATCTGTTAACAGGAATAACAGGTTTTAGCAGGGAAAGGATTA
AGGGTTTTATTTTCCTCAACTGAGGCCCAAAATGAGTGAGATTTTTCAAG
TTAACTTTCATTCCATTTGGCTCAGGTGTCAGGGATCTATATTTAACTTG
TGTTAGGCATGCGTCCGAAGAGCTGGAAGGCTGAGGATGTCAATGGCTTT
CAGATGTCAGGATAACCTTAAGGATAGATGAAGGGTTGAGAGCCTGTGCC
TCATTTCTGAGTTCTCAGCTGCTATGCCGTGGAAATCCTGTTTACTTTCT
GCATCTGCTCCTGCAAGACTCTGGAGCCAGTCTTGAGGTCCTACATCTCC
GAAAGCAAGCTCTTCTAGAAGTTGGTGAGCAGAATGCTTTTTGTTTAGAA
GTGTGGCTATAAATGTCTTTGTAGTTGATGCACTCTCATCTAGTAAGTAA
CTATTATTTTTGGGCTCTCTAGAAAGGTAATTATTATCTGATATAATAGT
TTAGTCTGTGATGCTTCTTTTAACATATTTGTAAGTTTTAACCAAATGGT
TAAAGAAATTTGCTTTTTAACCCTTAAACCTCACATATCCACAAGTCTCT
AAATTCCATAGGATGCTATGGATTTCTAGTTGCCTAGTTCATGTCTTTTA
CTTAGAAAACGTCAGAAAACCCAAACTTCTCGTGACTTCAAAAAGTGTAA
TTGTACCTGAAACTTCTTTTCCTTCAGATTTCTTATTTATGTTTTCTGAT
AGGTTTTTAAGATTAATCTTTTCAGAAGGATGCTCTAAAAATCTGGCCAA
TTTGATTATCCTCTTCCAACTTGGAAAAAATATGTATTTAAAATGAGACT
AGAATTTGAATGACCTTCTTTCATGGAACTCTGAAAATGTTTTTTGGCTT
GCTGTCACAACTGACAAATATATTTAAGAAATAACCTAAAAGACCAATCT
TAACAATTTGACTCCTAGACAGAAGAAAACAAAAATCTTTTTATACATAA
AACTTACTAATCATTAGTGATCATAGAAATTTTCTTGAGTGTGGGTGCCA
CATGTCTTATTTGTGGGGTGGGGGCAGATTTTGTTACATAGCAGAATTTT
TATTATATGGTGTGTTTCATAATATGGCTACATAAGATCAGACAACACCG
CCAGGAATGACAGAGTCGTTACTTATACAGAAAGTCAACTTAAGTTATGC
ATATTTCGAGACACAATTAAGGTTGATACAAAAAAAGAAAAGCAGTTCCT
TCTGTTTCTCCATGCTATTCTGCTATTCTACAAGCCCTGGAAAAGAATTA
GATAGGCTTCTTTGCCTTTACAAAAAAGGTCACTTTCTTTTATTATTTGG
GGAATGGTTGGTTGGTGCTAGACTGAATTCTGGGGAACCATTTAAGGCTA
ATACTTCCAAAGGGGTAAACTTGTTGACACTCCTTGAGAGAACTCACAAT
TCAGATGGGATCTCTATCTCAAAGAGAAATAGGCAACCTATCTTGTTTTC
AGGTGTAACTGACTAAATAATAAAAGTGGATTTGTGGGGCAATATATGAG
ACTTCTGTAATCCTGACAAGAGGAAAGGAAAAATATTTTCAAATGATGTA
CTATGCAAATACTAATTATTTGGAATCTGATACTTTTGTAAAGGATAATG
ATAACTGTGTAATTAACTGTTCTAAGAGTTCAAGAAAGCTAACACCTGAG
GATAATAAAACAGTTTTCACCTGGGGAAAAGATAAGCTCTGGAACTCAAG
AATTACAGTCAGGCAAAAAAAAAAAAAAAAAGAGTTTTAAACTGCTGAGA
AATGATTGAAAAAGACTGCTTAAGGTTGTAGAGTGGGATGATTAAATGGA
AGGAAATGTTTCAGAGCAAAGAAATATAAAGTTCTTGATGTGACTGCGGC
TCATTTTGCTTTTGGCACAAATTACTGATTAAGATGACTACTTAAGGAAT
TTAATCTGATATTCTTAGTATTTACAAACAAAGCTTCAGGTTAAATTGTT
TTGAAAAATAAAGAAATGCTATAAAATGAGTGAAGTGCACTTCAGATAAA
AATTGTGAAGTGTGTGCAACGCGCTTCACAACAGGTTACTTTATTGCCAC
CTGTGAGGAGCAAGAGGAGGTACTAGAGGAGGGGAATGAGGTTGGGTTTC
CATATCATGTGACTAGAATCCTCAGTCATGTCTAATCCACAGTGGTTCAA
T
Seq. ID. No: 95 CPT1B promoter 1
TCCCTCCTCACCCAGCCCCATTATTCTCAAGCCCTTAAGGCCACTCTTTG
CACAGAACCTTATATTTGGAAACATCTCCTGAATCCAACCTCCCCTGCCC
ACTGGGATGCCCAAGCGAGGCCCTCACCCGCTGAATTGTGGCTGACACCC
TGGGCACAGGAAGCTTGGGCAGAGATGTCTGGAAGCTGTAGAGCATAGGG
TGCCGGCTGGATAGAAGGCGGATACACATCTGGGGGTACAGAGCAGAGTG
CTGGGGTGGGAGCCAGTGGAAAAAATGGAAACCAACCAGCAACTCCCTGA
GACCCTCAGAGCCATCCCAGCCCCTCCAGGACAAACCACACACCAGGCAC
CAGTGCCCTGTCTGCCTCTCCCGTCTAGCTCAGAACCCCAAAGGAGTCCA
AAGAGTCTCAACCCAGGTGCCTGAACCCCTCCACTGGCTGCTGCTCACAG
CCCAGATCCTGGTCAAGTTGCTGGTCTTGCCATGCATCTCAAACATCCAC
CCATGGTAGCAGAGAAGCAGCTTCAGGGTTTGGCGGAAGAAGAAGATGCC
CGTCACCCAGACGCCCGTGGAGAAGATGGCCATGCTGAGAAGTGCCCGGG
TCTGCGGGGTCTGGTAGGGGCCACACCTATTGGAGAGGGGCAAGGGCTGC
AGGGGGTCCTCTTGAGGCCAGCCTCGGGTGGCTGTGAACTGTCTCAGGGG
AGACACCAAGGGGAAGAACCTCCCTGGGCACACTCATGTCTGACTGCATC
AAATGAGCGGATGTAGGGCTAAGACAATGGTTGTCATAGGGGAGGGCTGC
CCCGTGACTGCCAATGGCTGCTGTCCTGTATCTGCCCAGCTCAGGAACCT
CAGCCTTGGGAGCTGGGCCCAGCCCTCCCTGGTGGCACCTGTGCCCTTCC
AGTTTCACTCCTTACCCCTGAGGGAGGCATCTCTGGATGCAACTGACCAG
CCCCAAGGAGATGTCCACGTTGCAGAAGGAGGAACCCACTGTTGCCATGA
TGACGACCAGCCAGCTGGTGGGGCTGCCAGGGTACACGCCCCTGAGGATG
CCATTCTGTGGGCAGAAACAGGCGCCCTTATGGAGCCGGAAGGCGGGAGG
TTAGCCTGCCTGTGAAGTCTTGGGAACTGGCTCCTGGTCTTGGCCTGGAA
GGCTTTCTCTCCTGATGCCCTGCTGTGCTCCTTCCTCCGCCACACCCACA
GCTCCCTGTGTCCGACCACATCCTGTGCACCCCTCTGGTGTCTGTACTTC
CACAACCTGTACCGGGCAGAAGTGCCAGCCAAGGAGGCAGTGAGGGACCC
TAACAAAGCGCTTAACAGCTGACAGCCGGCCTCTGCCTACGCTCTGGGAG
AAGCACCTGTGCGCACCTTGATGCGGATCAGGCGTTTCTTCCAGGAGTTG
ATCCCAGACAGGTAGACGTGTTTCAGGGCCTCCCGACTGAGCCGGAAGTC
GACCCCGTCTGGGGTCACCGTGAACTGGAAGGCCACGGCCTGGTGAGCTT
CCGCCATCCTGGGGGTTGGTCGGCACCTAGGACGGGGGCAGATGGGTGCG
CGGGCGCGCTTAGGCCGGCCCCGCCGCCAGCCGCGCCGAGACGCCCCCAG
CCAGTCCGCGACCCCTCGCGCCCCCCACCCCGCGACTAGCGGCTGCCCCC
GGCCCGCGCCCCCCGCCAGGCCAACCGCCGCCAAATCCTCGCGCCAGCCT
TCCGGGTGGGCACAGCCACTGTGGTGCAGGGGATTTGGGCCTTGAAAGCT
CCAGGAGCCCCAAGGACGGCGAAGGGCAGCCTGGAGTTGGGGGGCGTCAG
CGAGGGCGCGGGCAGGGGAGAGAGGCGCCCCTGGAAGTGGAGCCGGCCTG
GACAGAGGCAGGGTTCGGGAGCCCGCCCTTCCCCACCCGAGCGCGCGGGC
CCCAGCGCCGTCCTCGCCCCTGTGCAGGGCTGAGGGCCCAGACCCCCGCG
GGCCGCCCTGTGCCGGCCCCTACTCACAGCTCGGGTTCACTCCTGTCCGT
GCGTGGGGCCAGCCACTCTGGCCCGTGTCCCCACGTCCTTCAGGCCTAGC
CACCCCCGCCCCGCCCTCACCAGGAACCTGACACCTACTCCCAAATCGGG
GTCGGGGAAAAACGTGGGGCGAGAACCAGAACCCACCCACCTTCCGCGGT
GGGAATTGCAGGGCGCGGCTGGGTTTCTGTGCGGTGAAGGTTGCTCAAAG
GTCTGGGAGCCTGGGGGGCCCCAGAGTCTCGTGAGGATGGTGGCTTTAGA
GCTAAAAATAGCCAAATGTAGGGAAAAGGTCACCGATGTGTCAGCTGCTG
CCTGTTCTGGGATCGCTGGTGCTCCGGGCGGCATCCCCACCTCAGGGTCG
TCTTCATACCCCCATCACTCTCCTCCTCCCCAAGTCCTAGTATTTATTAT
TCTAAGCTGCAGCCAGGGGACTGGGGAGGGATGGGGGACCCCAACTGGCA
GCATGAACGCTAACCACTTTCAATCCCAGAGCACTGAAGAGTCCCTTTGT
GTGTACTGCTCGGTTTGGTACTTTCTGCTGCTGCTCAGCAGGGCCAGGAC
AGGGTGGGAAGAAGAAGCTTGTTTATTGTGTTACATACACAGCACGGGGC
TCTGGCCTGCCAGCCATGGGGACCTACTCAAACTCAGGAACAGGCCGGTC
TCCTGAACCTCAGTTTCACTTGGGGGCTCAGCCCAGTCGCCAGGGCCTTC
TGCTCGTTGTTCCTCCCTCCAAGGTCCTGCCCTGGAGGCTCCAGGAGAAA
TCCAAGGAGTGGGAGGGTGGAGTCAGGATGAGGAGTGGACACTGGTCAGC
TGCCCCTTCTGCTGGAAGTAGAACTGGAACCGAGACTGGGCATAGTCCTA
GGGAAGGAAACCCACCCCACACAGTGGTGAGAAGACGTGGGACCAGCATT
CTACATCCACCTCAGGCTGCCCACAGCCACCCGGGACTCCCCAGGCCTCC
TGGAAGAGTGTGGCTGCTGCTGCTCCCCAGCCCCCCAGCTAGCATTCCCA
TCCCCAGGGTCACTTACCAAGTAACCAAATTCTATGGTGGACATGGATGC
CTGGAGGATGGACCACAGACCCCAGAAGAAATGGGATGCCAGAGCATACC
TGGGGGGAGGGCAGGAAAAGGAGGGGCATTCAAGAGCATGTCTAGCACTG
CTTGGATCCCCTCACCCAGGTTGGCCAATCCCTACAGTTTTAACTTCTCC
CCCACTGTCCTAACTCCACTCTCCACAGCCACCTTCCCTGCAGCTTGATA
AATCTGCCTCTTCCCCAATCCCTCTTTCCTCTGCTCTGCCCTACCCCACC
CTGCCCCTCCTTCCTCACCGACTGACTTCTACCAGCAAATCTTCTTCCAG
TTTTCTCTGCTCCTCTTGGGAGAGGGTCTCACCTTTCTTTGCCTCTGCCA
GGTAATGACGAATAAAATGCAACTACGATCAATGGCCAAGAGTCAGGAAT
TGGGGAGACTGTGGAGTATTTCCCAGGTAACATCCTTTCCGGCCATTCCT
TCCAGACTCCACCTCCCTCCTTCCTCACAGGATGGGTCCTGCTCCCCAGC
CTCTGGCCCACATACCTGCTGTTCTTGAGTGGGGTAGTCTGTGGGCCTTG
CTTTGTAGAAAGGCCATTCCTCGTGAGTATAATCATAAACCCACTCACAA
AAATGGTTCCCAATGTCAAAGCCCCTGGGAGAATAAGGTGGACATTCAGT
CCCCAAATGCCCTGGGCAGCTGGCCTGTTTTCAAGAGCCCTATGGGAACA
GATCTATGGGAAGCCATCTTTCCAGCCTCACCTATAGTTATAACTGCTGT
ACTCGAAGTCCACCAGCATGAGGCTGTCAGCATTTTCTGGCTCTGAGAGC
AGCAAGATGTTCCCTGGGGGAATGGGGTGAGGTTCTGCTCACTCCAGAGC
CCTCTGGCTCTTCCATCTTGGGTTAGGAGACTCAGATGCCTTCTCCTACC
TTCCTGGATGTCATTGTGGCAGAAGACGACTGGCGATGGGGTAGACTCTA
G
Seq. ID. No: 96 CPT1B promoter 2
CAGGCTGGAGTGCAGTGGCGCGATCTTGGCTCACTGCAAGCTCCACCTC
C
CAGGTTCACGCCATTCTCCTGCCTCAGCCTCTCCCAGTAGCTGGGACTAC
AAGCACCCACCACCACACCTGGCTAATTTTTTGTATTTTTAGTAGAGACA
GGGTTTCACCGTGGTCTTGATCTCCTGACCTTGTGATCCACCTGCCTTGG
CCTCCCAAAGTGCTGAGATTACAAGCGTGAGCCACTGCGCCCAGCCCATG
GTGTCTTATAAGTGTCTTATATAAGGGTACCATTTTTTTTTTTTTTGAGA
CGGAGTCTCGCTCTGTTGCCCAGGCTGGAGTGCAGTGGTGCCATCTTGGC
TCAGTGCAAGCTCCGCCTCCTGGGTTCACGCCATCCTCCTGCCTCAGCCT
CCCAAGCAGCTAGGACTACGGGCGCCCGCCACCATGCCTGGCTAGTTTTT
GTATTTTGTTTAGTAGAGACACTGTTTCACCGTGTTAGCCAGGATGGTCT
CGATCTTCTGACCTCGTGATCCACCTGCCTCGGCCTCCCAAGGTGCTGGG
ATCACAGGCGTGAGCCACTGCACCTGGCCATAAGGGTGCCATTTTTAATC
TTTTATATAACATTTTTACTATACCTTTTCTATGTTTAGACAAGTTTAGA
TACACAAAGACTTCCCATTGTATTACAGTTGCCTACAGTATTCAGTATGG
TAACATGTTGTAAAGGCATGCGGCCTAGGAGCAACAGGGTAAGCAGCATA
GCCCAGGTCTGTAGTAGGCTGCACCATCTACCTTTGTGTAAATGCACTCT
AGAATCTTTGCACAACAAAATCGCCTAACAATGCACTTCTCAGAATGTAT
CCCCTTCATTAAGCAACCCATGCCTGTACTTGGTAAGTGGATTATCCCAA
AAGCCTCCTGGTTTCCTGCCAGCTGCTCTGTCCCCTGACCACACCTGCCT
CACCCAGCAGTGAATTCGACATGGGCAGGAATAGGACCTTGTTTACCACT
GAATCCCCAGCACAGGGCCAAGTCTATGGTGGGTGTTCAACAGACACTGG
TTCCTGGCCGGGCACGGTGGCTCACGCCTGAAATCCTAGCACTTTGGAAA
GTCGAGGTGGGAGGATCACTTAAGCTCAGGAGTTCGAGACCAGCCTGGGC
AACATAGTGAGACCCTATCTCATGGGAAAAAAAAGAAAAAAACAAAAACA
GATGAAGACACTGGTTCCTAAATGAAGGTCCAGAACTGAATCTAATTCAC
TTCAATTACAGTAATTGAGGGCCTACTTATACTTTTCATAAAAATTATTT
TTTATGGCTGGGTGCGGTGGCTCACACCTATAATCCCTGCACGTTGGGAG
GCTGAGGCAGGTGGATCACCTGAGGTCAGGAATTCGAGACCAGCCTGGCC
AACATGGTAAAACCCTGCCTCTACTAAAAATACAAAAAATTAGCCGGGCG
TGATGGCGGGCGCCTGTTGTCCCAGCTACTCAGGAGCTGAGGTACGAGAA
TCGCTTGAACCCAGGAGGCAGAGGTTGTAGTGAGTCGAGATCGTGCCACT
GCACTCCAGCCTGGGCAACAAGAGCGAAACTCCGCCTTAAAAACAAAGAA
TAATTATTTTGTATTTACTCTTAACATGATTTTAGCAGCCGAGCCAACCT
CAGTGAAGGAGTGTCTGTTGCTACAGGTCATCCTGCGTGCCCTCTTGCAG
ACATGACCACCTGGCGCCTCAAGACTTCAGGCCTGGGCCTGCAGCCCCTT
TCCATGCTGACAAGAAGCTGCGGGGCCCGTGCAGGGTTGTGCCTCAGCCA
AGAGGGGTGCCTCACACATGCCCCTGCATGGGAGGAGGTGGGCTCACTCT
CCAGCAGACAGACAAACAGAAATGGCCCCAGGGAACAGGTGAAGGTTCTG
AGGCAAGTATTTATTCCAAGCAAACACGTGGGCGTGGACAGCTATCTCAG
AGCCTACGTCACTCAGCAGAGTCACACATTTCAAAGCAGGTCACACAAGT
CCCTCATGAACAGTCTTCCAGCTTCAGCCCTTCTCTTGCCACCTCTGTGG
TCTGAGCTGGGGGAGGGGGAGGGCCTCCGAGTTCCCAAACAAAACACAGG
TACCTAAGGGCTGAGAGAAAATTACACAGCCTTATTTTTATTAGTATCTA
TCAAGAAAAAAAAAAATCAAAATTCCCTTCCTGCTCCAACCCCACCTCCA
CCACCTGGGCCCTCAGGGAGCAGCATCTGCCCAGCGAGGACCTGCTGCCG
GAGCTGTCCTTGGAGCCTGGGCACCTGTGCACGGCCACCCCTCATGCCTG
TGAGCTGCCACAGGTCCCTTCCTCCACAGTGTGGGGACGAAAGGGCAGCT
GGCATTTCTCCAACCTTCAGCTGTAGGCCTTGGGAACTTGGAAAAGATCA
GCAATGTCCAGCAGGGCTTTGCGGATGTGGTTTCCAAAGCGCTGGGCGTT
CTGTGGGAGCCAAGAGTTCAGATGCACCTTCAGATATGTACCCACCCCTC
TGTTCCCACAAGCAAGGATGCCTCCCCAGCCAAAGACACCTGTGTTCCTG
GGCAGCCCCAGGTGGCACCCCTTCCTCAGGCCTGAGCTGTGGCAGGAGAC
TCACCGTCTCTGAGCTTGAGAACTTGCTGGAGATGTGGAAGAAGATCGTG
TTCTCGCCTGCAATCATGTAGGAAACTCCATAGCCATCATCTGCTACCTG
AGGGCAACAAGAAGGGTGAAGAAGGCATAAATCAAACCTTGAAGATCTGA
AAGCCAAGCTGATATTGTACTTGTTCTTCCCACAAGAGTTGCAGGAACGG
GCCCCAGCCCTAGACACTGCATTTTTCCTGAGGACCACCCCCAGGAAACT
GTGAAAACCAAAGAATGCTGCCCAGACCCCGCCAAGGGATCTGGGACCTC
CCAGTTTCCCTCAGGGGGCTTATTCTCTAGATCACTCCGTCTCTCTCCAG
AGGGTTCTCTAGCCTTCTGACAAGCTAGCCCATGTGAAGGTTTCTGTGGG
AGACAGCTGCACTCTCTTGGATAAGTAGACTTCAGAAGCACCCAGTGCAC
TGCCCAGTACCCTGGAAACCAGCCCCACAGTCATGCTTCAGTACCCCGGA
AACCACGGTCATGCTTCAGCAGGGAGGTATTTGGGATGGGTGGGTCCCAG
GTGCTTGGCTGCCTGAGTCCATAGCTAACATTCTGCTTCCATGCAGGTCT
GGGGTTATGCTCTCCTCTAAGCAAGGTGCTCCTGCCTCTCGTCTGACCTC
AGGGCCTGCACTCGCCACTGAAACCCTCAGGGCCCTGGTGCTGCCCACCC
ACCCCCTTCAGGAGCACTCACAGGGCCAAAGCCACCTCCAGCGCCCAGGT
GATTGGGGTGCTGCTCTGGGTCGAACATGCGGATCTGGGATTGGGGGATC
TGGCTGGTGGAGAGACGCCAGGGTTCCGAGAGCACCTGCAATGGAGGCCA
CAGCTGGTCAGAGGCCACATACCCAAAGCACCTGTCCAACACGTGGTGTC
TGCGACCTACTCTGAGCCAGACACGGTTCTGCTGAGGGTGCAGGAGCTCC
CTGGCCACCACCCCGTGGTCCCCTGTGCATCATGCCACATAAGGCTTGTG
TGAGCTGCCCAGCCCTCCCTCTGCTCTGCCTCTCCCCCACATAGCCTCAC
TAGTCTCAGTAGTGCTGTCACCACCTGACAGATCATATATTTGTCTGTTC
CCTGTCACAAGGACACCAACACCACAAAGAGGATGTTGCTCCTGCTCAGT
CCCAGGACCACAGGCCCAGGTGGGGTGTGGGCGGGAAAGCTGGCCCAGCA
CTCCAGGGAGCTTCAGGGGAGCAAGCCAGGAAAACAGGCCGTGCTCCATC
ATGAGATGGCCCAAGCCCTGCGTGTAGGAGGGCAGTGGGACAAAGGACAC
ACCCAACAACGGTGCTGACCTCAGCAAGGAAAGGAGAGCTGACTCCTAGG
TACTTGGAGACCAAGTAAAGGCAGAAGAGGTGCCTGTCGATCCCTGCCCC
G
Seq. ID. No: 97 AIFM2 promoter 1
AACCCGGGAGGCAGAGGTTGTAGTGAGCCGAGGTCATGACATTGCACTC
C
AGTCTGGACAACAAGAGCAAAAACTCCATCTCAAAAAAAAGAAAGAAAGA
AAAATTTCTAAAGTGATGTAAACATTTTTTACAAGTCTGTAAAGTATTGA
CTGAAAACCAGTGTAGTACAAATGTATATAGAAGTGTTGGGTTGTAAAGG
AGTGATGAGAGTGGAGGCTGACTGAAGAAACTCTTCAGGTGAGCTAAAAA
TAATAAAAACCCTTACTAGATCCAAAGAGTCGGGTTGCACTGCTACCCAA
AGTTAAGAAAATGCCCCCGCCCCCCTTTTCTTAAGACAGACCAAGACTCT
GTCTCCAAGGCTGAAGTGCAGTGTTGCCATCATGGCTCACTGCAGCCTCA
ACCTCCCAGGCTCAAGAGATCCTCCCACCTCAGCCTCCTCAGCAGCTGAA
AATGGCCTTGGTGGCTCCTCTCTCCTTAAGATCTTAGGATTTGTACCAAC
TCATCAGTAGTTTTACAATCTGCACAGTACTTGTACATCTCATTTCATCC
CCACACCAACCCTACCAGGCAAGGATCACTGGCCTCATTTAACAAGATAA
AGATGAGGATACTCCTAGTTTTGCTGAGTCCTTTAAGACGGCTCAGTTGA
AAAAGTAATAGTGCCAGGGCTTAAACCCTCATCTCTAACACCAAAGCCTA
AGCTCTTCCTGTTAACAGTACATAAAATTCAAACCCAAACCACAAAATTG
CTGGGGAAAAAAAAAAAAACTAAAACTTCTTTAACCCACCAACTACACAG
AGGATGATCTTCCGAAGTTCTCTTCCTTACAACTCATCACATCTTTTGAA
TGGTATTTTAATAACTAGCATTCTATTTATTTTCTATTCTTGGTCCATTG
TACCACCTTACGAGAAGTTAAGAGTAATCTAAAATTTAAGGTAGGTCTCT
AAACTATCAACAAAACACTATTAATGTACCTAAAATGAAAACTTGCGAAT
TCCAGGGCTAAATGATTCAAACAAATGTTAATAGAAAAGGAGGGCACCAA
TCTGACCATTAGCCAATTTAAAAATTTACGCATTACTTGTTATACATAAT
CAGGGCAACTTTAAGACGCCATTCCAGGCAGATTTGGGGTAGTAGGCGGC
TGGCTTTTAGCTAACTCTGCTTAGCTGCGGGGTAAATGTCAAAGGTCAGC
CCCATGGCGAATCTCCCTAAGCCCCCTGTCGCAACCGAAGCCGATGGAAT
CAACAAGACAAAGATGCTCCTGACCCACTGGCTAGAATCGTGAAGAAAAC
GCTAATCCATGTCAAATGCACATAAGGAAATTTTTCCATGCAGAGACCAG
ACAGGGGAAAAGGGGAAGGGAGAAGGAGAAAATACAGGGTTCGACCCGGG
CCATCGGAAGACAACCAAGCGCTGACTTGGGGGTAAGCCCTGGACTAGGG
GCCTGTAGACCAGGTTCGAGCGCTCACACTCGCTAACTGCGGAGCCTTGG
CCAGCCTCAGTCTCCGAGTCTACAAAACAAGGATAATCATAAAGGGCTCC
CACCCGCCTCACCGAGGGCTGTGAGGCTCTTCCGAGACAGAGGACATGGA
GCCATTTCGTAAACCGGAGCGCACGGTACAGACAAGAAACTGTTATCCGG
CCCAGCAGCTGCGAGACCCCCGAGCGGAATGCAAGTGGACCCCAGGCAAA
GAGGACAGCAGGGGACGGAGCAGATCTGGCCAAGTTGTCCCGTCCCCGCC
CAGAATCAGCCCCGCGGCCCCACAGAACCGCCTGCGTGTCACTTCCCCCC
CACCCAACCCCGCCAGGGCCAGCCTCCGCTCCGACCTCCCCGCCTTCCGT
CCCAGCCCCTCGAGCCCCAACTCCCCTCCCCGACGCGACCCTCAGACTCA
CGGCCTGAGGGGCTCCTCCGGCAAAACAGCGGCTGGCTCGGACCCTCCCT
CAGAGCACACTAACCAGCAGCACCCGGGACCGCCAGCTACTCGCCGGATG
TACGTCACACCCCTCCCCGGGACTTCCGGGCGCGTAATGGGCCCCGCCCC
CTCACAGCGTTACGCCTCTCTGCCCCGGTGCCGTCCCACCAGCGACTCGG
GCCGCGGAGGGGGGGGCATAAGGCGTGACAAAGCGCGCTCGGGGTCTGGC
CCCGCCTCGGCCCCGCCTGCTCCCGTCTGGCTAGCTGACCGCGCGAGACG
TGGCAGCGCCGGAACCCTGGGTCCGGGGCTGGGTCCTGGAGTCCCTGAGC
GGCCTGGTACACTCGGGATCGCGGCTTCCCATTCCAGACGCACCAGGAAA
GTCAGAAGTGGGTACCGACGGTGACATAGGTCCTGGGAAAGATGAGTTGA
CCTTGGGGCCCTCCACTTGAGGTGGATCCCTCCAAGCTTTTGCTTTTTTT
CTTCCTTGCTTTTTTTTTTTTTTTTTTTTTTTTTGAGGCGGAGTCTCACT
TTGTCGCCCAGGCTGGAGTGCAGTGGCGCGATCTCGGCTCACTGCAACCT
CCACCTCCCGGATTCAAGCGATTCTCATACCTCGGCCTCCCGAGTAGCTG
GAACTACAGGCGCATGTCACCACACCTGGCTAATTTTTTTGTATTTTTAC
AGAGACAGGTTTCACCGTGTTGGCCAGGCTGGTCTCGAACTTCTGACCTC
AAGTGATCCGCTGGCCTCGGCCTTCCAAAATGCTGGGATGAGAGGCGTGA
GCCACCGCGCCCAGCTGCTTTTGCTTTGAGTCGCACTTCCTTGCCCTGGG
GTGGCCTATGTTTTAGAAAAACAGAAAAACAGCTAATATTTATTGTGCAC
TTATGTTCCAGGCAGAGCACTGGGTACTTTGCACGCTTTGTCTAATTTCA
CCGTCATTGGAGTCCTGTGACTCCAGATGCTAGTATTATCCTTATTGACA
AATGAGAGAGCAATGTTCAGAATAACCTACCATGCCCAAGTTCACACAGC
TAAGTGGCAAAGCAACGTGTTCACCCCAGTATATTTCGCATGCATTTTCT
CATTTAATTCTCAGGACAGTCCCACGAGATTGGTACTATTATTACTCTCA
TTTTACAAATAAAACAGTCTAATGGAAAGATGAATGACGTCTGTGGGTTC
ATGAAGATCTCAATTCTCCTTTAGTAAAATTTAAATTTAACATAATCCCA
ATAAAAGTACTAATAGGATTAATAGGTCGTGTGTGGTTTCGTTCTTTTTT
TTTAATTACCCTATTTGTTCCTAAAATTCGTATAGAAAACCAAACAAAAC
TAGTCCAGAAACAGGCTTTCAAAATGAAGAACAAGGAGGTGGGAACTAAC
CCTGTTGGATATTAAAACATATTATAGAGCCTAAGTTATTAAAACTGGTG
TGCGTGCACATGCACAAACACACACACACAAAAAACACAATGGAACAGAG
CAAAGTACAGAAATGACTAAAGTACATGCAGAAATAAAACTAAAGTAGCA
CCTGGGACTCCAGCAATACTCAGGGAGCTGAGTGGGCAAGCTCACCACCC
TCTACCCTCCGCTCATCCTGGGGTCTTAGATGTTGAATTTGAACCCTGTC
CTGTCACAGAAAGGTGTCAGGCCACTTCTGAGGCTTCTCACCAGCTTTAG
CTAAACCAGGGACAATGTGTCCATGGGGCTTGGAACCCCTTGTGCATCCT
GGAGACACAGATGCAAGCCCAGTGCAAGCCAACACATTGGATTACTGGCT
TTGTTGAGAGCCTGGCTGCACGAGGTGGAGGTGTCTGAACCACAGGATGG
CTTGGGAGGCTGGGGAAGGAGGGAACATGCAGGCAGATTCTCAGAAAAGC
CGCAAACTCTACTTACCTAAAGGCTTGAGCAAGTTGACGAATGCCCCAGA
CCACCATGTACCCACTGTAAAATGGGTGAAACAGGCATTGTTGTGCACAT
AGTTTTTCCAGAGTACTGTTTTCAATAAATGATTCTGTTATGTGTTGTTA
AATAAATTAATAAATAATAAAAGGGGTTGAGACAATTGGGTAGTCAAAAA
A
Seq. ID. No: 98 AIFM2 promoter 2
CTCACCTCGCCACCCTGCAGCAGCACCATCTGGTTCTTCAGGTCTATCCC
CACTACTAGCCCCTGCCGGAAGTTGTCCTTGAAAGTCACCGAGTAAGAAA
TGAATGTCTTTTTGGCGAACCCTGGGGAAGGGACACAGAAGAGGTCATAG
GGCAGAGCCAGGCTGCCCACGGGAAGCCAGAGTGACTCAGAACCTTTCAC
GAGCTGCAAACCAACAGGGGTGCCCTTGACCAAAGCGTCGGGCCATTAGG
TATGCCTGGGGTACCCCTACTTCCTAAAATGCCAATCTGGGCCTCAGACC
GTCAGAAAAGCTCAAGCTTTTACTTCTAGGGTCTCATTGTGGTGGCTACT
TAAGACCAAAGACCTTGAGCTTCTCCAGAACTTGTCTGACAGTGGAATGC
AGGCACTTGGCCCTAAATGGAGAAAACCACCCCCAGCCCTAAAATCAAGC
CCCAGAGCAGAGACAAGACCCCCCTCACAGAGACAGCCCCAGACGGGAGC
ACATTACCTGTCTCCACGGAGGCTCGGAGAGCAGCCACATTGTGGTGGAA
GGAGTCCTTCATGTCCACCAGCATGAAGGGGACGTTCAGGGCCTGCAGCT
GGCTGGCTGCTGCGATCCCGCCAAAGCCCCCACCCACAATCACCACGTGC
AGAGCTCCCGATTCCACCGAGACCTGGGACCCCATCTCAAATCAGGCACT
GCTGGGAAGAAAGAGGAGAGCATATCAGAGTCAGGGAGGCTGGGAGGGCT
GGGAGGACCCACAGTGAGAGAGGCCTCGATCATGAATTGACCTTTTTAGA
TGAGGTAAATCTTTGATTAATGAAACTCCAATTTTATAAAATTTGAATTA
AATTCAGAAGGAACAAGGGGGTATCCCACTTCCAGCCCATTCCCCAAACA
CCAAGTTCCCTTCTCCAGAGGCATCCACTGTTACGTAACAAGTTTGGATG
ACTCCTTCCAGAGATAGTTTATGCAACTATAATCACGATTATTTTTTGCA
CCCATTAAACAAAGTGTTCTGCACATTTGTTTTTTACTTGATATATTCCA
TCAAAGACTTTTGGGAGAGATTGCAGTGGGGCAGGAGGAAAAAAAAGACT
TTTGAAGAGGGGAATAATGTTCACTCAACAACATATATGAATTGAACTCC
TGCTTTTTGACATGCTCTGTGTTGTGTATCCTGTGGTCAGACAGGCAGAT
ATGGTCCCTGCCCTTATAAGGGCAGACCTTACAAAGTCAAAGAACCAGGT
GGGCAATGTCAATGATGTGAGAAGCTGGGAAAAGGTGCAATAACAAGAAA
CAGTGGGCACACCTGTGGGGAACCAGCAAGCATAGGCCACATCTAAAGGA
ACAACAAAAAACATTCTTTAAGCACTGCACTAAACAAACACCGCAACCAG
ATTCAGCCCAGCTCACAGGTCACCAACTTAAGGTCCAGGTCTAAGGAGGT
CAATTAGCAGGTTACGACAAGAATCCTGCAAAATTCTGCTTCTAGGGTTT
GAATATGTCTCCCAAAAAGGAACAGTGTTAAGAGGTGGGGCCTAATCGGA
GGTGTTTGGGTCATACGGTCTCCACCCTCATGAATGGATTAATACCAATT
ATAAAAGGGCTTTGAGGCTGCAAGTTCAAGCTCTTCCTCTCTCTCGCTCT
CTCTTTTGCCTTCCACCATGGGATGACTCAGCATGAAGGCCCTCACCAGA
CACTGGGCCCTTGATCTTGGACTTCCCAGCCTCCAGAACCATGAGCCAAT
ACATTTCTGTTCATTATAATTACCTAGTCTGTAGTATTCTGCTGTAGCAG
CAGAAAACAGACTAAGACAAATGTGGTGTGAACTAAGACTGTGATCATGG
AGTAGGAAGGAGGGGCCACAACCATATGCTACAATGCCTGGCTAATTTTT
TAATTACTTGTAGAGATGGGGCTGGGTGTAGCAGCTCACGCCTGTAATCC
CAGCACTTTGGGAGGCTGAGGAAGGAGGATCTTTTGAGCCCAGGAGTTCC
AGACCAGCCTGGGTAACATAGAGAGACTCTGTCTCTACAAAAAAAAAAAA
AAAAAAAAAAAAAAAAAAAAAAGCCAGGCGTGGTGATGGGCACCTATAGG
CCCCGCTAATTGGGAGGCCGAGGTGGGAGGATCACTTGAGCCAGGGAGGT
CAAGGCTGCAGTGAGCCGTAACTATGCCACTGCACACCAGCCTGGGCCAC
AGAGTGAGACCCCATCTCAAAAAAATAAAACAATAAAAATAATAAATATT
GGTAGAGATGGGGGTCTCCTTTTGTTGCCCAGGCTGGTCTCAAACTCCTG
GGCTCAAGTGATCCTCCCACCTCAACCTCCCAAAGTGCTGGGATTACAGG
CATGAACCACTGCACCCAGTCCCCATTTCTTTGTATGACCTCAAGATGGT
ATATAAGCTTCTGTACCTCTTTTGGGGGTCGGGTCTTCCTTCTGAAGGCT
CCTGTGTATACACACATTAAATAAATTTATATTTTTATATTTGTATAAAG
TTGCCTTTTATCCAACTCAACAGCCTTGGGCCCTACAGCAGCAAAGTTGC
CTGGGCTCCCCAAGCCAGCCTGGAAGGCTGGTAAACAGGGTTACCTGTGC
ATCCCAGAGTCTGCCCCAGACAGAGGAGGGCCCAGGGGCTGGCCTGGGGC
TGCACCAGAGGCCTTGCCTCCAGCCCAGCAGAACCCCAAATTAGGATGCT
TGCAGTGTGGGCCTCAGGCAGGCAGAGGGCTGCTGGTGTGGGAGGAGCCG
CACAGCCCATTGAGGGGCACTCTTGACTGCAGCTGAGACCCTGAGAGGTG
ACAAGCAATCAACCCTCCTCCTGGCCTGTGCTGAGCCCTAAAGAGAGGCA
GAAATAAGCCAGGCTGCTGGGGGGAGATGCTGAGAAAAAGAAACAAAACT
CACACTATACAGAAAGCAGCCCCAGAGGCTCAACAGCTGCTGTGGGCTTC
AGGAAGGGGGACTGGAGCGGGTGTCAGCGAAGGAGGAAGTAGACTCTGAG
GTCAACCTTGAAGGATAGACATCAAGCCACTGAACAGCCTCATAAGGAAG
TGTGTTCCAGCTCATCTGGGACCCTCAGGCCGCTGGAAGGCCACTGCACT
GCCCTGATCATCTTTGCGGGTCTGGGGCACAACCCCTTCCCTCTCTAGGC
CTCAATTTACCCCCTATAATGGGAGGGGACTATAAGGCCCAACCAGCTAT
AATATCCTGAGCTGTCCTCGTACACACTGGTTTCCAAGACATGGTGGTGG
TTCCCCAAGACGGTCCCCAAATCATCCCACACTTATGGGGCAGCTTTTGG
TTAGAACTCCGGGCACTGTTCCCATTGGTGTTCAAATGCCCACTGCCTAA
CTTCTCCCCTGGGGGCGGACAGTAGAGCGGACGGAGGGCACTGACACAAT
GGATTGGAGGGAATTTAAAGTTGTTTCTCGTGGGTCAAATGGTCCCCAAA
GCATTGAGGCTTGAACATACCCATTTCACAGATGAGGAGGCTGAGCCCTC
GTAGAGATTAGGACCCAAGACAACCCCCCGGCAGGCAGGGCCACCCTGGC
TCCTCTAAGCCCTCAGAAGGGTTGTCTAGAGCTAATCCCAAGCGTACCTC
AGGCTGAATCTGCCAACTGGGCCAGAAGGGAATGTCTCCTAGGGACCAGG
ACCCAGGAGGGCCCACAGGCCCAGCCAGAGGTAGCAGTGCACACCAGGAG
CTGCGCAGGGCCTGAGAATGTAGGCAGTGATGAGGCAGGCCGTGGGTGGC
GTGTGTTTCACCAGCCCCTGGCAGCGTGCAACCCTCTGTGCTGGGTCTTA
CCCTTGGGGCTGTCAGCCAGGGATGAGGTCAAGAAAACACCCAGAAAGCC
CCACTTCCAAGTTAGCTGGGCATGGCCCCTGAGAGGGGCTACGCAGCTGG
CCGGGGATAGGGACAGGGGAGTCCACTGTGCGTCAGGTCACACTGGAGCA
CCCAGGGACACTCAAAAAGGACAGCCTAGAGCCCTGAAAAAGAGCTGAGA
TCAGGGGGCCTTCCTAGCAGAGGAAACAACTTGGGCCAGAGGCCCAGTGG
CAGGAAGGGAGGAGGCAGGTAGAGTTCAGGGCACCTGTGGCAGAAGGTGG
AGAGGCTTAGAGGACAGGTGACAGGGGCCGACAGACAGACAGCCTGCCAT
GCCAGGGCAGCTTTGACAATCAGGTGGACATACAAGCCTGCTCCGGGCCC
TTCATGGGGTTTCCTCCAGGGAAAGAAAGTCTAGTGACCCCAGAGTGGAC
CTCAGGGGCAATGAGTTGACCAAAGGAGCCAAGCCCTTGACCCCCAGGCC
CCTAGTATCATCAACCTGCTTCCCCGAAAGGGCAAACTCTTGCCTCACCC
AAGGAAGGCAAATTGCTTATCTGGGGTTCCCTGTGAAGGGCCTGGGGCCT
GAGGGGACACCCAGACCAGAGCAGGGCCAGGGAGGGGAGGCACTTTTCCT
CTTCAGGCTGCCTGAGGCCTCTCCTGCAATCTGCTCTCTCTCTCTTCTCT
CTCCTCTACTGACCCCTGCCACGCTTACTTGTGGAATAGGGAGGCCAGTG
ACTCTCCAGCAGATTTCACCCTGCCTCTTTGGCAGCCCCTTGCCCAATAC
TCTTGGACATGCCGACTCTCTTTGGCTCTGCCAGTGTCGGTTCTTGCCTG
GCCCCAGAAAACAGTGAACTCCCCTCTGCTTGGCCATGATCTACTTGGCC
AGACACCTAAAGCAAGGGCTTAAGCCATGGAGCTGCATTTCCAGCCACAC
ACCCTGTAGGAGATGAAAATGCACGTATGGAATTAGTGGATGCCCAGTTG
TATGTTCTAACATAGCAGTAGTGCTGTGCAAACCTCCACCTGTGTGACAC
TCCATCAATCAATGGAAACTGCCACCAATGAGAGGCTACAGGATAAAATA
TTATAAACCCATACAATGCAGTACTGTGTAGCCAGTTGTTTTTAAAGGGT
GAGTGATGTTTTGTTTTGTTTTTGAGATGGAGTCTCACTCTATTGCACAG
GCTGGAGTACAGTGGCTCAATTTCAGCTCACTGACTGCAACATCTGCCTC
CTGAGTTCAAGCAATTCTCCTGCCTCAGCCTCCCAGGTAGCTGGAACTAC
AGGCATGTGCCATCATGCCCGGCTAATTGTTTTTTGTATTTTTAGTAGAG
TCAGGGTTTCGCCGTGTCGGCCAGGCTGGTCTCGAACTCCTGACCTCAAG
TGATCGGCCTGCCTCGACCTCCCAAAGTGCTGGGATTATAGGTGCGAGCC
ACTGTGCCCAGCCTGTGAATGACGTTTTTATACAAAAAGTCTGTCCATGA
AAAAGTCAGGCAAGGGCTGGGTGTGGTGGCTCCTGCCTATAATCCTAGCA
CTTCAGGAGGCAAAGGCAGGAGGACTGCTTGAGCCCAGGAGTTTGAGGCT
GCAGTGAGTAACTTTACCTTTGCTCCCTCCCACAACACACACACACAAAG
ACACACATACACAATCTGATTTACACAAAACATGTTTTCAAGGTTCATCC
AAGTTGTAGCATGGATCAGTGCTTCATACCACTGCACTTCGGCCTGGGCA
ACAATGTGAGACCCTGTCTCAAAAGTAAAAAAAAGAAAAGAAAAAGTCAG
GCAATATTTATTTATTTATTTACTTATTTTTAATTTTCTTAGGGACAGGG
TCTTGCTCTATCACCCAGGCCAGAATGCAGTGGCATGATCATAACTCATT
ATAACCTTGAACTCCTGAGCTGGCTAATTTTTTTTTTTTTTTTTGTAGAG
ATGAGGGTCTCACTATGTTGCCTAGGCTGATCTTGAACTCCTAGCCCCTC
AAGGGATCCTCCTGCCTTAGCCTCCCGAAGCACTGGAATTACAGGGGTAA
GCCACTGTGATTTCGAGCTGTAGAAAAGATTCAATGAGATCATTTACGTA
AAGCATTTTAGCATGGTGCCTGGCACAGAGTAGCAATGAATACACAGCAG
CCACGTTTTATATATATATATATAAAATATATGTTGCAGATTAGTTTGCC
CAGTCCCAAAAAGTCAGGCCACAGAACCACATGAAAGATCTAATCCCATT
TATGGCCAATAAGACACATCCACCTCGGTATGTATACACATATACAGGTA
ATGATTACAGCAGTTAGCATCTGTTGCACTTTTGCCCATATCTTCGATTT
AAGGTATGAGTTTTTTCTCCTTATTTTGGAAAAAAGTTCAACAACTTGCC
CAAGTTCAATTCTCAACACATCAAACCTGCTGTCTGAACCAAAGTGCTAA
TAGTGGTCATAGATAATGTTTTCATTTTCTTCCTTAAGCTTTTGTATTGT
TTGAATTTTTTTCAAACAAGCATATGTCATTTTTGCAGAAATAATGTCTT
TAATAATAGGACAGACCGCTTACCATCTAATTTTTTAATCAAAATAGAAT
TCACATATACAATTTACCACTTAAGATGTAAGTTCACAGTGAGCTATGAT
TGCACCACTGCACTCCAGCCTAGATGAGACCCCATCTTTTAAAAAAAAAA
AATGAGATGTAGGCTGACTCACACCTCTAATCCCAACACTTTGGGAGGCC
AATGCAGGCAGATTGCTTGAGCTCCGGAGTTCCAGACCAGCCTGGGCAAT
ATGGTGAAACCCCATCTCTACAAAAAATACAAAAATTAGCCAGGCATGGT
GGCACATGCCTGTGGTCCCTGATATCAGGAGGCTGAGGTGGGAGGATCGC
TTGAGCCTAGGAGGTCGAGGTTGCAGTGAGGCATGATCATGCCACTGCAC
TCTAGCCTGGGCGCCAGAGCAAGACTCTGTCTCAAAAAAAAATTTTTTTT
AATTTAAATTTCAAAAAAAGATGTAAGTTCAGTGGGTTTTAGTGTATTCA
CCAGGTTGTGCAACCATCACAACTATCTAATTCCAGAACACTTCCATCAC
CCCCAAAATAAATCCTGTACCCCACCTACCCTAGCCCTGGGTAGCTACTA
ATGTTCTTTCTGTCTCTATAGATTTGTCTGTTCTAGACAATTCATGTAGA
TGGAATTATACACTATGTGGCTTTTTGTTTGCGACGTCTTCCACTGATCA
TCATGTTTTCAAGGTTCATCCGAGTTGTAGCATGTTATCAGTGCTTCATT
CCTTTTCCTGGCTGAAAAACAGTCCATTACTGGTGTATGAATAGACCACA
TTTTGTTTATCCACTCATCAGCTGACGGGCACTTGGGTTGTTTCCACCTT
TTGACTATGTGAATAACGCCACTATGAACATTTGTGTACAGGTTTTTGTG
TGAACCAGTCTTCAGTCCTCTTTAGTATATATTTAGGTATGGAATTGCTG
GGTCATATGGTAACTTGAAGTTTAACTTTTTGAGGAACTGCTAAATCCAC
CATCTGATTTTGAACTGAACACCAAAGCCCCAGTGGGAATCACAGAGAAT
CTTAACTATGTCTGTCTAGGTTCAAGAATGTTAGCTTAAAAGAAGGCCTA
AGAGAGCGGAACAAACCCTGATTGTCCAGGAACCCCTCAGTAACTTTACC
TTTGCTCCCTGCCACAACACACATACACACAGACACACACACACACAATC
TGATTTACACAAAAATTTGCTCAGTCAATCCTGTTTTCTCTGCCAAGAGC
ATCCACCCTGATCTCAAGGGTATTATTCCACAATAAGCAGCCCATAGGAC
TACTGAAAAACCAGGAAGTTACATTTATGCCAAAGCTGTTCAGAGGTTGT
GCCTCAGAAGACACACAGCAGGAACCGTGTGGGCTCTGATCAAAGCAAGA
ATGTCATCCCAGAGTTGCTCCAGTATTGCTTCACAAATATTACAAATAAA
ACACGATGCTTTTTGAGTCGTCATGGCTCACACAAAGGGTAGGTTTCAAC
ATCAGCCTCTGGAGAGCCACATTCCCAGAGTGGCCGTCCCGGATAAGAAG
GCCACTACAGCACGACTCACAGCACCATGGAGGTTTCCTCCACCTGCGGG
ACACAGCAGGATGGAGACGGTTGGGCGGCCTCCCTTCCTGGGACACTCCA
TGGCCAGCCCCTCAGGGACTACTTGAAGAACAGAACCCCCAAAAGACCAG
TTTCTGAAAAGGGACTCAAAGTCTAAGGGAGGCAGCCTCCTTTTATCCCC
AGGGGAGGGCAGCTCTGGGGCAGACAGGTTCATCTGGATGTCATATTCAC
TCCCGCACAGAAAACCTAGGCCAAGCAATTTGTCCATCCTCCTCAGAGCC
AGTTTTGTGGTCCCCATGGTCTGCCCACCACCTGTACCTGGACGCTGGCT
TCATTTGGCCACCAGACAAAAGTCAGGCTGGCCCTGGAGTCCACCGGCCT
GCCCAGCCCATCACTTCTGGGCTAGGTAGTGCTAAGCCTGCCCAGCCCAC
CCTGGCTGGGGCTCAGTGCCCTCTTCTTGGTGACACACTGTGCTCCTTGG
AGAACTCCTCTCTAGGTGCTGGGGTGAGGGCAGGTGTGATCTGAGATCCC
GGGGCTCTCTGGCAGGTGCATGCACGCACACTGGAACCTGATACAGGCTT
TTGAACACAGGTGCACAGTCCAGAAACCTGCAAGTCCCATCCCAAACTAT
ATCTGACTCTCTGCCCTATGCCTGTCCTCCTACAGGATCATCAGCCCCCA
AGAGACCTCACCACTCCCCATCCTCACCTTAAATTCCAGGGTCAAAAAGA
CTCTCCTTGTAGGGAAAGGTAGGGTGTGTTGATCAGGTGGTGTTGGAAGG
AGAGCCCCAATATAGGCCACTAATTCAGTTCCAGGTGGACTGCACGCGTC
CCATCAGAGTTAGTTTACTCACCGCTGGTCTTTGTCCCTGCAGCACCCAG
GGCTTCGGGAAGGGAGAAGCAGACTTCCTAGTGCTGCACCCCCCAGCTCC
CCCGAGCCTATGGGCACTGGGTTGACTCCAGTTTCTTTCCTCTTAGAGAC
TGAGGTTTCTCCCTTTCCTCTCACACCTGCTTCTGTCCGGACCGGCCGCT
TCTACCCAGCGGACCCTCGGACGGTCCTCGCAAGATCCGGGTTACACCCG
CCCCGTGTTCCCTCTTCCCCGGTTCGCCCTTCCCGGCCTAAGCCCATCTT
CCTTAGGAAAACTTAGCAGGGATTTAGGGGCCAAACCAGGAGGGTGGAGT
TTTCTTTGCCCTCGGCCAGGAGCACATCTCCCTGTTGCGTTTTTTGGGGG
AGACAGAGAGGGGCGGGCCTGAGTCGAGTATCCCTCTCCTCGCAGCGCCA
CAACTGCGCGCACCGACCTCCCCACGCCAGACGCGGCGTGAGCCAGGCCT
CTAATCCGCTCGCCCCTCTTCCGGGTCCCGGTCCCGCCCGGGGCGCCCGG
GGTCTCGAGAGCCCGCCTGGCGGCGCGATCTCACCTGTCGGCCGCGCCCG
CGCGCTCTCGCTCCGGCTCCCGCTCCCGCTCCCGCTCCCGCTTGGTCGTC
TTCCCGAGTTACTGACCCGAGGCGCTCCCGGGCGGGGCGTGACTCAGCAC
TCGTGCTGGTCCCCGACCCCGCGGCAACGCCCCGGCCAGGCCCCGGAAAC
GGTCCTGGCACGCTGGGGCTGGTCTCCGAACGGCCCGCCGGACTAACCTT
GACCCTGAGCGAACGGCCTTGAACTTTGAAACAAATGCTCCCATGGCCAG
ATGTCGCCTCTCCGGCTCCTGCCAGCACCCGCGCCCCAGTCCTGCCGAGC
CATTCAGACTTGGTTTAAAGTGTCCTTTCAGCATACTTTAAAGCAAAAAA
GAGAGAGAGAGAGAGAGAGAGAGAGAGAGAGAGAGAGAGAGAGAGAGAGA
GAGAGAGAAGGAAGGAAGGAAAACAAGCATCCCCCTGTTCCTCTGATTTT
CTCGAGGACACCTTTAAAGCCACTGGAACCTTGGAGAGTGGGGTTCCCCT
CACACGTTGGGCGCACTGCCAACATCCTTCCTTCAGACATACACATAATA
ATTCCGCAAACCCTGTTGAGCTCCTACATTTGCCAGGCACTGTGCTAGGC
GTTGGAGGGATAACTTGGAGAGAAAACTGACAAGGTTCCTGCTTTCACGG
AGGCGACAGTCTGGGAAAAGACAGATATTAAACACACCACCAAAAAATAC
ATGTATAGAGAAAACAAAAACCATGTATCACCAACTGCAGTAAGAAAGAT
TGGGCATGGCCAGGCACGGTGGCTCATGCCTACAGTCCCAGCACTTAGGG
AGGCCAAGGCGGGAGGATCGTTTGAGCCCAGGAGTTGGAGACCAGCCTGG
GCAACATAGCGAGACCTACAAAAATTAGCCGGGCTTGTTGGTGCACACCT
GTAGTCCCAGCTTCTCGGGAAGCTGAGGTGGGAGGATTGCTTGAGCCCAG
GAGTTTGAGGCTACAGTGAACCATGATCATTCCACTGCACTCCAGCCTAG
GTGACTGTGAGATTCTGTCTCAAAAAAAAAATAATAATAATAATAATAAT
AAATGAAATGAAATGAAAAAATAAAAAAGAAAGACTGAGGGCAAAGGAAG
GGTGCAAATACTTCTCTCCAAGGATGAGAAAGAGGTAGATTTAGGTAAAG
AGGAAGGAAGTGTGGTCTGGATGTGCAAAGACCCACAGGCAGGACAATGA
CCTGGAAGGCAGTGTATTGCCAGCTCAAATCAGACCAAGCTAACTCAGCG
AAAGATAAGCCATCTCCATCTACTTACTGCAAATATTAGCACCTACTTTA
AGAGTTGTGGATAAGGGCCAGGCACGGTGGCTCACGTCTGTAATCCGAGC
ACTTTGGGAGGCCGAGGTGGGTGGATCACCTGAGGTCAGGAGTTCGAGAC
CAGCCTGGCAAACATGGTAAAACACTGTCTCTACAAAAAATTAGCCTGGC
ATGGTGGCACATGCCTGTAGTCCCAACTACTCAGGAGGCTGAGGCAGGAG
ATTCACTTGAACCAGGAAGGCAGAGGTTGCAGTGAGCGGAGGTCATGCCA
TTGCACTCCAGCCTGGGCAACAAGAGCGAAACTCCATCTCTAAAAAAAGA
AAAAAAGAGTTGTGGAAAGGCCTCAATGAGATCATTTTTATAAAGTGTTT
TAACATGGTGCCTGGCACACAGTAGCAATGAATACACAGCAGCCATTTAT
TATAGATATTTTATATTGCAGATTATTTTGCCCAAGGCAACCAGAATGAT
TTATTTTTGATGAAAAATCCTACTCATCGTCTTAGAAAAACAATAAGCTA
ATATTTATTGATCACGAACTAAGTGCTTTGAATGACTTCTCAAGTTTTTT
TGTTTGTTTTTGTTTTTTTCACAAAGGTCCTGCAAGGTAAGTTTTATTAA
TCTCCATTTTGCAGATAAGGAAATTTAGGCTCCTAGAGGCTAAAGAATTT
AGGCAAGGTCCCTCAGCTAGTAAAGCAAGGGGGTGGGCCTACAATGGAGG
TCTGAACCATGACCCCTTTTTGGGGGGTAGGGGGACGAATCTGGCTCTGT
CGCTTAGGCTGGAGTGCAGTGGCATGCTCTCGGCTCACTGAAGCCTCCAC
CTTCCAGGCTCAAGCGATTCTCATGCCTCAGCCTCCCAAGCAGCTGGAAT
TACAGGCACCTGCCACCACACCCGGCTATAGTAGAAACGGGGTTTCACCA
TGTTGGCCAGGCTAGTCTCAAACTCCTGGCCTCAAGTGATCTGCCCACCT
CGGCCTCCCAAAGTGCTGGGATTACAGGTGTGAGCCACTGAGCCCAGCCT
GAACCATGACCTTAAAGCCAGGAAACCCCTCAGCAAGCATCATCCCTAAG
GAGCCAGAGGGCACTGCTCAGGCAAAGCAGAATCTCAGTTGTGGGGAATT
TACTGGGCATGTACCCAGTAAGATGTGACACAGAGAGGTGAACAGAGAGG
CTACAGCCTGCCCACTGCACGGAATCAGCCCTGAGGCTTGGGATGGTCAT
GGAAGGCTTTGTGTAGAAGGTAGGCGTGCAAGGATGAAGGGGATGCCAGC
TGGCAGGATGCAAGGAAAGGACTCCAGAAAGAGCAGGACAAAGATAAAGC
TGGAAGAGAGGCGAGAGAGGTGTGAGGTGGCCCAACACTGGGCTCAGGTG
TGCCAGGGCTGGTGTGGCCCATCTCTAAGGACCTGTCCCAAACCATGAAA
AACTTTTTTTTTTTCCTTAAGACCTGCTACAAAGCAACAAACCATGAAAA
TCAAGGAATTTTTCTGATTATCACTCAATACTTTCTTGGCAACCAGAAGA
CACCTCCATCCAGGAGAGGGGACATGGTCTCATGCAACCCCTCCAGGTAG
CTCACTGCACACCACTCCTCTGGTCCCACTCAGCAGTGCCCATAGCTTTG
GAACTGAAAATCAGGGCTCGCTGCCTAAAAAAGGGGTTTTGTCTCTGAAA
TTCCAAGTGCCAGAGGTAGCAGGAAAGTGCGGTGGTTAGGATCCTGAAAA
AGTGGCAAGTTCATTCCATGGAACTTTGACGGTTAGTGGGGAGAGCAGGC
AGATTGTCTAACATGACGGAATCTAAGAGGAGGAAGCGTTGCTGGCTCTC
TGCTGTAATGAGCTCAGCCAGGCCTGTGGGTACTTCCTTACAGACAACAG
GTGGCTTTTCTGCTGGCTCCCAGGGCTGGAGGAGCTCCAAGGTACTGGGT
CATCTCAGGAGCCCAGAGAAACAGAGGCAGTATGCCCACTCGAAGAACAG
GCAACTCTTAGATCTGAAGAGCCTGGTAAATGTTCTCCTTTTCCATACGT
AGTTCCAACGAAATTTGTAAGCTTCAAAGAAGAGAGGAATATCCTCGTTT
CTTCCTCTCTCCCCCAACACATGATTTGGTATAGTGTTCCCAGCTAGGGA
GAGGCTATATTAATGAAGGTAAGTTCTGCTGCAATGACCAATAGATTCCA
AATGTAATGGCTCAAATACAACAGAAGTGCACTTCTTGCTAGGGCATGAG
TTCCAGGCCAGTGGACTGGTTTATGAGGCTGTCTCCCTTCACACAGGCAT
CCAGGGACCAGGCTAACAGGACATGGCAAGTGAGTGCCACTCATGGTCCC
AAATTAGATCTTTGTTTTTCTTTTTTAGAAACAGGGTCTCCCTCTGTCAC
CCAGGCTGGAGTGCAGTGGCATGATCATAGCTCACTGCAGCCTCGAACTC
CCATGCTCAAGCCATCCTCCTGCCTCAGCCTCCCAAGTAGCTGGGACCAC
AGGTGTGCACCACCATACCTGGCTAATTTGTGTGTGTGTGTGTGTGTGTG
TGTGTGTGTGTGTGTGTGTTTGAAGAAACAGGGTCTTGCTATGTTGCCTA
GGCTGGTCTCAAACTCCTGGGTTCAAGCCTCCCAAAGTGCTGGGATTACA
TGCGTGAGATACAGCACCTGGTCTCAAATTAGGGAGCTCATCAGTTCTAC
CTCCTGTGTTGTCACTTGATCTCACCACCACCAGTCCTGCCTCAGCCATC
TTCTCTCTCACCAAGCCACTGTAACTGGTCCCTGGGCCTGTCTTGCACTT
TTCCAATTTTCTCTCCACAAGGAAGCTGTGCCACACTTCCTTGTCCCAGC
TCCTGCCACTCTCAGCCTGGAACACTCTGTTTCCAGTCCACTTGGTAAAC
TCTGAACTCTCTTTTCATGGTTCAGAGCCTGAATCACCTTGAAGCCTTAC
CTAACCCACCCCTTCTGGGCCCCCAGACATACTCTTCCCTGGACAGAGCT
TCCCTGCTTATTGCACTAATGGGCTGACACATCAGTCTCCCCAGAGTAGC
CTGTGGGTGCCTTGATGCCTGTATTAGTCCGTTTTCACGCTGCTGATAAA
GACATACCTGAGACTGGGATGAAAAAGAGAATTAATGGACTTACAGTTCC
ACATGGCTAGGGAGGCCTCCCAATCATGGCGGAAGGCAAGGAGGAGCAGA
TCACGTCTTACATGGATGGCGGCAAGCAAAGAAAGAGAGGGCTGGTGCAG
GGAAACTCCCACTTTTAAAACCATCAGATCAGTGAGACTTATTACGGTCA
CAAGAACAGCACAGGAAAGACCCAGCCCCACGATTCAATTACCTCCCAAC
AAGTTCCTCCCATGACACGTGGGAATTGTGGGAGTTACAATTCAAGATAA
AATTTGGGTGGGGACACACCCAAACCATATCAGTACCCACACGTGCAAGA
ATAAACCCTCCTCCCCGCCCACCGCAACTCCCTCTCACAGCAAAGTCAGA
CCTCAGATCTAGGATTCTGCTACCATTAACTTACTGTAGAACTTCTCTTA
AACTAGCCATGACCTCTGGACCCATTCCTAATCTGTAAAGGGAGGAACAA
CCATCCCAGGAGTGTTCTGTGATTGCCCCGTGTTTTAGGAAAAACTCTCC
TCAAATCGTGTTTTCCCTCTACTCTCATGCCATCACAATCATCAACACAG
AAGACTTCAGTGACCAAATGTGTGTGGGGTTTCCCCACCAAAAGCAGTGG
ACATCAGCCGGGTGTCCTCTAATTCAGTTCCAACACTATCTACCCAGAGA
CATTGTCAGACCCCACAGGTTGAGGGTTCAGTCTCCAAGACTGCCCCCCT
CAACCAC
Seq. ID. No: 99 GSK3B promoter 1
GCCAGAACATTTAACAGGTAATGAATGCAGTAGCACTCCAAAATTGAAC
T
TAAATGTAATGAGCCAGTGACAACATTTAACTTAGTATTTGGCACTATGA
CAAAACAAATCTCAATCAAAACTTGGACCTATTAATACTTCCAAGTTAAC
TTTTGATTAAACATAAATTATACGGTGGCAGGTTGGTAATGTCAAAACAG
GAAGGTTAAGAGATAACACATCAAACCACTTATCCAATTGAACACAAATG
CATACAGAGATGTTACTGCTGCTAGGTATTTATTGGCTCCCACTAGTCCC
TTCTAAAGAGAGAAATCAATGGCAGCCTCCAAATGTGAATGGTTACTGAG
GAAGTTCATCCACAGATTAAGAACAAATATTATGTGTAGTCATACAAACA
AGAAAAAAATGTTGGGAGGGTGGGACATTTAACGTCTCTATTAAGTATAT
CAAACTATGATAACCCCTGAGACCTAAAATATTCTCTCATTCCAAATTCC
AAAAATTCTGCAAATGTGCATAATGGTTTAGTAATCAAATCTTGTCCTTC
TCTTTATTCTAAAGGATGGGCAAGTAAACACTGTCATCACTACTTTAACG
TGGAAACACTCGAGCACTTGATGAAACTGACTACCAACAGCAAGAAAAGT
ACAAGAAAGCAGTTTGAAATCGAATCATCCAAATAAATATCATATTATCT
CAATTCAAGGTTAATGAGACCGGGGGAGGGGGTATGGGAGGAGTAAAAAG
GGAGCTGCTTCATCCTTGACTGAAGATAGGTTTTGCCCTTTTCCATTGCC
TGTTTATCAGGAGGTCTAATAATTTCAGATCCTGGTATTTCGAGGTTTCT
TATTTTAAGGGCGAGGTGGAAAAGGGGTGTAAAATAAAACCAATACTCAC
TGCTAACTTTCATGCTGCCAAAAGCTGAAGGCTGCTGCACCGGCTTGCAG
CTCTCCGCAAAGGAGGTGGTTCTGGGCCGCCCTGACATGATCACTCTCTT
CGCGAATCACCTTTTCCTTCCTTCCTCCTTTTCTTCCTTTTGTCTTTATG
TTGGGGTGTTAGGTTAACGATAAATGCAGCATTAAGTTCTCCCACAGAAG
AAAAAGAAAAAGACTTCGTCCTCTTGGCTTTTCACTCCTTTTGTATACTA
TAAAAAACAAAACAAGCGATATATTTCTCCTTCAAGACAGATCGGCACGG
ATATTTAACATATAGATGATTTAGGACTTGGGAAAAAATACAATTCTTTC
CCCTCCCTTTCCTGGGAGGAGAGAAAAGAGAGGGCAAATCCTAAAAAAAT
ATGTATCACGTGAAACGGGGGCAAATACTTGATTGAAAATGAGTACTTCG
AATATTATATTTTCCTCGGGGATTTTTTTTCCGAGTCAATGAAGAAGGGA
AGCGGCGGAAGGATCACGAGTCTCACGCTTGAAGAGAAAGGGGGATGGGT
AGGAGGGAGGGAGAGGGAGGGAGGGGGTGGCTCGGAGATGCGACGGGAAA
CGCTGCAGCTCCGGCAAGCCGCGGGATCCGGCGGGCTGACGGCAGGGGCC
CGGCGAACTAGAGGGCGGCGGAGTCGCGAGTCAGTCAGAGGCGGGCGGTG
GCGGTGGCGGCGGCTCCTCTCCTCATTGCGCCAGGAGCAGCTGCAGGCGG
CGGCTGGATCCAGCGGCCATGGCGGTGGCGGAGGCAGCTCCCTTCAGACC
CCAGGCAGCGGCTCCTCGACTGTTCCCCATTCGCCGGGGACATGGGAACC
CGGCAACCGCTTCCGTCCCTCGGGGCCCCCTCCCCCGTCCGCGGCAACAG
CTCGGCCGCCCTCCCGCCCCTCGGCTCTGCTCGGTGCCCGCTCAGGAAGT
GTCCGCGCTTTGCCCCAGCCCAGAGCCCTGTCAGCGGCTGCGGGGCCGGC
TCCTCCTCGCTTCCTTCCTTCCTTTGTCACTTGGCCCGGGCGGCGGCGGC
GGCGGCGGCGGCACAAGCCCGCATTCGCCCGGGTCAGGAGCTGCTCTGTG
TGAGGAGCGCTGTCTGCGCAGCCGCCTTGCACTTCCCCACTCCTCCTCCC
CCCTCCTACTCGTCCTCCACCTCCTTCCTCCCCCACCCAGCCTTACACCG
CCCAGCGCCCCGCCGCCCGCGTAACAGGCGTCTGGGAATCGCCGGCCGAG
CCCCACGCTCGCCCGCTTCGGCTGGGAGCCGAAGCCCGAACGGAGCCAAT
CACGGACGTCGTTTGTTTGCGGCTCCTCCCGGAAAAAGCCGATCAGCCTG
AGAAACCAATTAGAAAGTGACGCTGGGAAGCCACGCCCAAAGAGCGGAAA
ATGCCGAAGGTAGCCGAACGGAAAGGGTGAATTCGCCTTGTTCGGTGGCT
GGCTGGAGGAGACATTCGACAATGGCAACAGTGGCCAATCAGAGTCGCCG
GCCCTTACGAAGAGGCTTGAAGAGTTGAGGACGGTGAAACCCAGAGACGC
TGGTGAAACTGCACGGCTGGGCGCCGCCGCCGCTTTGTGATTGGCTGGAA
AACTCCGCTGGGAGCGTACCTCCTTCGGTGATTGGTGAAGAGAAGGGGGC
TTCCGAGGACAGTCTGGCCAGTCAGGACTTCCCCTCCCTCTCCAAGGTGG
GGAGAAGAAAGCGAAGGGAGGGCGTCTATAAAGCGCTGTCTCATTGGCCT
GAAGCTTAGGCGCTGGAGTTGGGGCAAAAGGGAGAGGGAGGGAGCCCGAA
ACTCGCGGCGGCGGCCAATCGGCTCTCACTGCCGTCCAATCGCAAGACCT
AGCTGCAGTGTGATTGGCCTTCGGCCCCGGTCCCAGGAGCGGGGAGATGC
CTGACTCGGGTCGTGGCTGACGACCCGACGGTTCGCCTGCGGCCGCCTCC
AACAACGGTGGAAACCGCACACAGGGACCGCGGGCCCTCGGTGCAGCAAA
TCGGAAAGTGCTGCGGTCGGCGGCCCTGGGCTCTGAGCGCTCAAGCGAGC
AGAGGGGTGGAGGGGCTCTCCCCACCCACAGGAGGCTCTCGGCGGGGCGC
AGCCAATTCTGCGCGCCCAAGGCTCCCCTCTGCCGTGGATCGCGTGTGCC
GAGTGTTGGCCTCTTCCCCGAAAGGGCCAGACGGACCAAGGGGGCCTTCC
CAGCCGGCTGCAAGCTCTCAGACGCTAAAATGGGAACAAAGCTTCCAGAA
AGGCAGCGCCTTTGGGCGGCCGCGCCCCGCCCAGCCGCGGGGGCCATTGA
TACAGAGGCTTGCTGCGCTTGGTGTGCGCGGCCCGGCCGGGCCTCTGGAA
ACGCCCATGAGAAATGAGCTCATGCTGGATGCGCGCGCTCCTTGGGCGCA
ACTGCCCAGCGCAAAACGGGCCCCGAAAGCAGGTGAGGGGAAATGCCGTC
AGGCCGGATATCCTGAAGCCTCCGAGGAAATGGGGGGGTCAGAGGAGCCC
GTGGTAGCTTTGGATCTTTTGGAACTAGGGTGATCTATAAAAGTGCAGCT
TGAGAAGATTTCATACATAAGACCAGCGTCCATTGTTCTACCATAGAACC
AGTTCCTTTGGTCTGCTTTAGAACAGTGAGGTATGGCTACGTCAAGATAA
CAGAGAAAGTAGAACAGACAGTTGCCATCCTGATTGTAATCCAGTGGTGC
CTGTAATTTCTACCTGCAGAGTCATCTCTTTCTGTTAAGATGCTGTCTCC
AGAAAGCACATGTTAAAGGACCTATATTTGATCAGATTACCTGGCTCTTG
CTTTGAAACACTGATGAAGGACTTGGGACAGAACAAAGTAAGCTTTGATG
GAAGACAGGAAAGGTCTAGATAGTGTTTGCAGATTCAAATGGCTACAGAG
GCCAGACAAGTAAAGCAAATGAGTGAGACTGGCCTTGTGTAAGACAATGG
GGAATGATAGGGAAAATGGCAAATTGGAATATCCATGCCATAACTAAAAG
GAACAGCTGCTCAACTCTAGCAGATTGTTGCCCTCAGGAATGTGGGTCCA
GTGTTCTCAGATGTTCAATTTTTTTCCTCCCAAAAGAAACCCCAAAATTG
G
Seq. ID. No: 100 GSK3B promoter 2
CAAATATCCCTATTATTTCTAGGACCATAGCCATCTGCAAAAGCCAAAA
T
GGAAAGGACTAAATCCACATTCTTCTAACTAAATCCACAGTTATCTAAAT
ATGATGGAAACTAACGCTAGAGTGTTTTAAATCGTCCTTTTTAAGGAACC
CGCCAGAACATTTAACAGGTAATGAATGCAGTAGCACTCCAAAATTGAAC
TTAAATGTAATGAGCCAGTGACAACATTTAACTTAGTATTTGGCACTATG
ACAAAACAAATCTCAATCAAAACTTGGACCTATTAATACTTCCAAGTTAA
CTTTTGATTAAACATAAATTATACGGTGGCAGGTTGGTAATGTCAAAACA
GGAAGGTTAAGAGATAACACATCAAACCACTTATCCAATTGAACACAAAT
GCATACAGAGATGTTACTGCTGCTAGGTATTTATTGGCTCCCACTAGTCC
CTTCTAAAGAGAGAAATCAATGGCAGCCTCCAAATGTGAATGGTTACTGA
GGAAGTTCATCCACAGATTAAGAACAAATATTATGTGTAGTCATACAAAC
AAGAAAAAAATGTTGGGAGGGTGGGACATTTAACGTCTCTATTAAGTATA
TCAAACTATGATAACCCCTGAGACCTAAAATATTCTCTCATTCCAAATTC
CAAAAATTCTGCAAATGTGCATAATGGTTTAGTAATCAAATCTTGTCCTT
CTCTTTATTCTAAAGGATGGGCAAGTAAACACTGTCATCACTACTTTAAC
GTGGAAACACTCGAGCACTTGATGAAACTGACTACCAACAGCAAGAAAAG
TACAAGAAAGCAGTTTGAAATCGAATCATCCAAATAAATATCATATTATC
TCAATTCAAGGTTAATGAGACCGGGGGAGGGGGTATGGGAGGAGTAAAAA
GGGAGCTGCTTCATCCTTGACTGAAGATAGGTTTTGCCCTTTTCCATTGC
CTGTTTATCAGGAGGTCTAATAATTTCAGATCCTGGTATTTCGAGGTTTC
TTATTTTAAGGGCGAGGTGGAAAAGGGGTGTAAAATAAAACCAATACTCA
CTGCTAACTTTCATGCTGCCAAAAGCTGAAGGCTGCTGCACCGGCTTGCA
GCTCTCCGCAAAGGAGGTGGTTCTGGGCCGCCCTGACATGATCACTCTCT
TCGCGAATCACCTTTTCCTTCCTTCCTCCTTTTCTTCCTTTTGTCTTTAT
GTTGGGGTGTTAGGTTAACGATAAATGCAGCATTAAGTTCTCCCACAGAA
GAAAAAGAAAAAGACTTCGTCCTCTTGGCTTTTCACTCCTTTTGTATACT
ATAAAAAACAAAACAAGCGATATATTTCTCCTTCAAGACAGATCGGCACG
GATATTTAACATATAGATGATTTAGGACTTGGGAAAAAATACAATTCTTT
CCCCTCCCTTTCCTGGGAGGAGAGAAAAGAGAGGGCAAATCCTAAAAAAA
TATGTATCACGTGAAACGGGGGCAAATACTTGATTGAAAATGAGTACTTC
GAATATTATATTTTCCTCGGGGATTTTTTTTCCGAGTCAATGAAGAAGGG
AAGCGGCGGAAGGATCACGAGTCTCACGCTTGAAGAGAAAGGGGGATGGG
TAGGAGGGAGGGAGAGGGAGGGAGGGGGTGGCTCGGAGATGCGACGGGAA
ACGCTGCAGCTCCGGCAAGCCGCGGGATCCGGCGGGCTGACGGCAGGGGC
CCGGCGAACTAGAGGGCGGCGGAGTCGCGAGTCAGTCAGAGGCGGGCGGT
GGCGGTGGCGGCGGCTCCTCTCCTCATTGCGCCAGGAGCAGCTGCAGGCG
GCGGCTGGATCCAGCGGCCATGGCGGTGGCGGAGGCAGCTCCCTTCAGAC
CCCAGGCAGCGGCTCCTCGACTGTTCCCCATTCGCCGGGGACATGGGAAC
CCGGCAACCGCTTCCGTCCCTCGGGGCCCCCTCCCCCGTCCGCGGCAACA
GCTCGGCCGCCCTCCCGCCCCTCGGCTCTGCTCGGTGCCCGCTCAGGAAG
TGTCCGCGCTTTGCCCCAGCCCAGAGCCCTGTCAGCGGCTGCGGGGCCGG
CTCCTCCTCGCTTCCTTCCTTCCTTTGTCACTTGGCCCGGGCGGCGGCGG
CGGCGGCGGCGGCACAAGCCCGCATTCGCCCGGGTCAGGAGCTGCTCTGT
GTGAGGAGCGCTGTCTGCGCAGCCGCCTTGCACTTCCCCACTCCTCCTCC
CCCCTCCTACTCGTCCTCCACCTCCTTCCTCCCCCACCCAGCCTTACACC
GCCCAGCGCCCCGCCGCCCGCGTAACAGGCGTCTGGGAATCGCCGGCCGA
GCCCCACGCTCGCCCGCTTCGGCTGGGAGCCGAAGCCCGAACGGAGCCAA
TCACGGACGTCGTTTGTTTGCGGCTCCTCCCGGAAAAAGCCGATCAGCCT
GAGAAACCAATTAGAAAGTGACGCTGGGAAGCCACGCCCAAAGAGCGGAA
AATGCCGAAGGTAGCCGAACGGAAAGGGTGAATTCGCCTTGTTCGGTGGC
TGGCTGGAGGAGACATTCGACAATGGCAACAGTGGCCAATCAGAGTCGCC
GGCCCTTACGAAGAGGCTTGAAGAGTTGAGGACGGTGAAACCCAGAGACG
CTGGTGAAACTGCACGGCTGGGCGCCGCCGCCGCTTTGTGATTGGCTGGA
AAACTCCGCTGGGAGCGTACCTCCTTCGGTGATTGGTGAAGAGAAGGGGG
CTTCCGAGGACAGTCTGGCCAGTCAGGACTTCCCCTCCCTCTCCAAGGTG
GGGAGAAGAAAGCGAAGGGAGGGCGTCTATAAAGCGCTGTCTCATTGGCC
TGAAGCTTAGGCGCTGGAGTTGGGGCAAAAGGGAGAGGGAGGGAGCCCGA
AACTCGCGGCGGCGGCCAATCGGCTCTCACTGCCGTCCAATCGCAAGACC
TAGCTGCAGTGTGATTGGCCTTCGGCCCCGGTCCCAGGAGCGGGGAGATG
CCTGACTCGGGTCGTGGCTGACGACCCGACGGTTCGCCTGCGGCCGCCTC
CAACAACGGTGGAAACCGCACACAGGGACCGCGGGCCCTCGGTGCAGCAA
ATCGGAAAGTGCTGCGGTCGGCGGCCCTGGGCTCTGAGCGCTCAAGCGAG
CAGAGGGGTGGAGGGGCTCTCCCCACCCACAGGAGGCTCTCGGCGGGGCG
CAGCCAATTCTGCGCGCCCAAGGCTCCCCTCTGCCGTGGATCGCGTGTGC
CGAGTGTTGGCCTCTTCCCCGAAAGGGCCAGACGGACCAAGGGGGCCTTC
CCAGCCGGCTGCAAGCTCTCAGACGCTAAAATGGGAACAAAGCTTCCAGA
AAGGCAGCGCCTTTGGGCGGCCGCGCCCCGCCCAGCCGCGGGGGCCATTG
ATACAGAGGCTTGCTGCGCTTGGTGTGCGCGGCCCGGCCGGGCCTCTGGA
AACGCCCATGAGAAATGAGCTCATGCTGGATGCGCGCGCTCCTTGGGCGC
AACTGCCCAGCGCAAAACGGGCCCCGAAAGCAGGTGAGGGGAAATGCCGT
CAGGCCGGATATCCTGAAGCCTCCGAGGAAATGGGGGGGTCAGAGGAGCC
CGTGGTAGCTTTGGATCTTTTGGAACTAGGGTGATCTATAAAAGTGCAGC
TTGAGAAGATTTCATACATAAGACCAGCGTCCATTGTTCTACCATAGAAC
CAGTTCCTTTGGTCTGCTTTAGAACAGTGAGGTATGGCTACGTCAAGATA
ACAGAGAAAGTAGAACAGACAGTTGCCATCCTGATTGTAATCCAGTGGTG
CCTGTAATTTCTACCTGCAGAGTCATCTCTTTCTGTTAAGATGCTGTCTC
CAGAAAGCACATGTTAAAGGACCTATATTTGATCAGATTACCTGGCTCTT
GCTTTGAAACACTGATGAAGGACTTGGGACAGAACAAAGTAAGCTTTGAT
GGAAGACAGGAAAGGTCTAGATAGTGTTTGCAGATTCAAATGGCTACAGA
GGCCAGACAAGTAAAGCAAATGAGTGAGACTGGCCTTGTGTAAGACAATG
G
Seq. ID. No: 101 GSK3B promoter 3
ACCCCGGAATTTTAAAATATTTTTCAAATCACTCCATGAAGTGCTTAGAA
AGCACGACCCTTCCCTGAAAGATGCATAATCAGCTTTCCTCTTTACCTAC
CCGGGCATTCTCTACTTGCAAGCCACAAGAAAAAAAAAAAATTAACTTTC
AAAAATTATCTCCCATTCAGCTATTACCTAGACTACCCTGTTTCAGGCTA
GTTCCCAAAGCCAACATGAATTGAAATCTTGAGTTCAACTAAAAGTGATC
ATTTTATGTTGCCTAAGAATATAATCAATTTCACATAACATCATGTTTTG
CGTGTGTGTGGGTTCTTAAAATCTCAGTTCTTTAGTTCTCTCAGTTCTTC
TAGGCAACAAATTAATTTGTCTTTGCCCTCATGATGCCCCTCTTTCCCAC
AATAAGGAAACAATATAGATATTTTTGTTAAATTCTGCAAGTATGTTTAA
ACAAGCTATCATTAAATACCACATGAACTTCTCCATAAATAGAGATACAC
TGGAACATTAGATACTTAGTATATATTAAACACTTCAAGAGCATACTTTT
CAGCACAAACCCAGGACAAGAGTAGTCTATCCACAACCCTGAACACAGCC
CAAATATCCCTATTATTTCTAGGACCATAGCCATCTGCAAAAGCCAAAAT
GGAAAGGACTAAATCCACATTCTTCTAACTAAATCCACAGTTATCTAAAT
ATGATGGAAACTAACGCTAGAGTGTTTTAAATCGTCCTTTTTAAGGAACC
CGCCAGAACATTTAACAGGTAATGAATGCAGTAGCACTCCAAAATTGAAC
TTAAATGTAATGAGCCAGTGACAACATTTAACTTAGTATTTGGCACTATG
ACAAAACAAATCTCAATCAAAACTTGGACCTATTAATACTTCCAAGTTAA
CTTTTGATTAAACATAAATTATACGGTGGCAGGTTGGTAATGTCAAAACA
GGAAGGTTAAGAGATAACACATCAAACCACTTATCCAATTGAACACAAAT
GCATACAGAGATGTTACTGCTGCTAGGTATTTATTGGCTCCCACTAGTCC
CTTCTAAAGAGAGAAATCAATGGCAGCCTCCAAATGTGAATGGTTACTGA
GGAAGTTCATCCACAGATTAAGAACAAATATTATGTGTAGTCATACAAAC
AAGAAAAAAATGTTGGGAGGGTGGGACATTTAACGTCTCTATTAAGTATA
TCAAACTATGATAACCCCTGAGACCTAAAATATTCTCTCATTCCAAATTC
CAAAAATTCTGCAAATGTGCATAATGGTTTAGTAATCAAATCTTGTCCTT
CTCTTTATTCTAAAGGATGGGCAAGTAAACACTGTCATCACTACTTTAAC
GTGGAAACACTCGAGCACTTGATGAAACTGACTACCAACAGCAAGAAAAG
TACAAGAAAGCAGTTTGAAATCGAATCATCCAAATAAATATCATATTATC
TCAATTCAAGGTTAATGAGACCGGGGGAGGGGGTATGGGAGGAGTAAAAA
GGGAGCTGCTTCATCCTTGACTGAAGATAGGTTTTGCCCTTTTCCATTGC
CTGTTTATCAGGAGGTCTAATAATTTCAGATCCTGGTATTTCGAGGTTTC
TTATTTTAAGGGCGAGGTGGAAAAGGGGTGTAAAATAAAACCAATACTCA
CTGCTAACTTTCATGCTGCCAAAAGCTGAAGGCTGCTGCACCGGCTTGCA
GCTCTCCGCAAAGGAGGTGGTTCTGGGCCGCCCTGACATGATCACTCTCT
TCGCGAATCACCTTTTCCTTCCTTCCTCCTTTTCTTCCTTTTGTCTTTAT
GTTGGGGTGTTAGGTTAACGATAAATGCAGCATTAAGTTCTCCCACAGAA
GAAAAAGAAAAAGACTTCGTCCTCTTGGCTTTTCACTCCTTTTGTATACT
ATAAAAAACAAAACAAGCGATATATTTCTCCTTCAAGACAGATCGGCACG
GATATTTAACATATAGATGATTTAGGACTTGGGAAAAAATACAATTCTTT
CCCCTCCCTTTCCTGGGAGGAGAGAAAAGAGAGGGCAAATCCTAAAAAAA
TATGTATCACGTGAAACGGGGGCAAATACTTGATTGAAAATGAGTACTTC
GAATATTATATTTTCCTCGGGGATTTTTTTTCCGAGTCAATGAAGAAGGG
AAGCGGCGGAAGGATCACGAGTCTCACGCTTGAAGAGAAAGGGGGATGGG
TAGGAGGGAGGGAGAGGGAGGGAGGGGGTGGCTCGGAGATGCGACGGGAA
ACGCTGCAGCTCCGGCAAGCCGCGGGATCCGGCGGGCTGACGGCAGGGGC
CCGGCGAACTAGAGGGCGGCGGAGTCGCGAGTCAGTCAGAGGCGGGCGGT
GGCGGTGGCGGCGGCTCCTCTCCTCATTGCGCCAGGAGCAGCTGCAGGCG
GCGGCTGGATCCAGCGGCCATGGCGGTGGCGGAGGCAGCTCCCTTCAGAC
CCCAGGCAGCGGCTCCTCGACTGTTCCCCATTCGCCGGGGACATGGGAAC
CCGGCAACCGCTTCCGTCCCTCGGGGCCCCCTCCCCCGTCCGCGGCAACA
GCTCGGCCGCCCTCCCGCCCCTCGGCTCTGCTCGGTGCCCGCTCAGGAAG
TGTCCGCGCTTTGCCCCAGCCCAGAGCCCTGTCAGCGGCTGCGGGGCCGG
CTCCTCCTCGCTTCCTTCCTTCCTTTGTCACTTGGCCCGGGCGGCGGCGG
CGGCGGCGGCGGCACAAGCCCGCATTCGCCCGGGTCAGGAGCTGCTCTGT
GTGAGGAGCGCTGTCTGCGCAGCCGCCTTGCACTTCCCCACTCCTCCTCC
CCCCTCCTACTCGTCCTCCACCTCCTTCCTCCCCCACCCAGCCTTACACC
GCCCAGCGCCCCGCCGCCCGCGTAACAGGCGTCTGGGAATCGCCGGCCGA
GCCCCACGCTCGCCCGCTTCGGCTGGGAGCCGAAGCCCGAACGGAGCCAA
TCACGGACGTCGTTTGTTTGCGGCTCCTCCCGGAAAAAGCCGATCAGCCT
GAGAAACCAATTAGAAAGTGACGCTGGGAAGCCACGCCCAAAGAGCGGAA
AATGCCGAAGGTAGCCGAACGGAAAGGGTGAATTCGCCTTGTTCGGTGGC
TGGCTGGAGGAGACATTCGACAATGGCAACAGTGGCCAATCAGAGTCGCC
GGCCCTTACGAAGAGGCTTGAAGAGTTGAGGACGGTGAAACCCAGAGACG
CTGGTGAAACTGCACGGCTGGGCGCCGCCGCCGCTTTGTGATTGGCTGGA
AAACTCCGCTGGGAGCGTACCTCCTTCGGTGATTGGTGAAGAGAAGGGGG
CTTCCGAGGACAGTCTGGCCAGTCAGGACTTCCCCTCCCTCTCCAAGGTG
GGGAGAAGAAAGCGAAGGGAGGGCGTCTATAAAGCGCTGTCTCATTGGCC
TGAAGCTTAGGCGCTGGAGTTGGGGCAAAAGGGAGAGGGAGGGAGCCCGA
AACTCGCGGCGGCGGCCAATCGGCTCTCACTGCCGTCCAATCGCAAGACC
TAGCTGCAGTGTGATTGGCCTTCGGCCCCGGTCCCAGGAGCGGGGAGATG
CCTGACTCGGGTCGTGGCTGACGACCCGACGGTTCGCCTGCGGCCGCCTC
CAACAACGGTGGAAACCGCACACAGGGACCGCGGGCCCTCGGTGCAGCAA
ATCGGAAAGTGCTGCGGTCGGCGGCCCTGGGCTCTGAGCGCTCAAGCGAG
CAGAGGGGTGGAGGGGCTCTCCCCACCCACAGGAGGCTCTCGGCGGGGCG
CAGCCAATTCTGCGCGCCCAAGGCTCCCCTCTGCCGTGGATCGCGTGTGC
CGAGTGTTGGCCTCTTCCCCGAAAGGGCCAGACGGACCAAGGGGGCCTTC
CCAGCCGGCTGCAAGCTCTCAGACGCTAAAATGGGAACAAAGCTTCCAGA
AAGGCAGCGCCTTTGGGCGGCCGCGCCCCGCCCAGCCGCGGGGGCCATTG
ATACAGAGGCTTGCTGCGCTTGGTGTGCGCGGCCCGGCCGGGCCTCTGGA
A
Seq. ID. No: 102 IRS1 promoter 1
GTGATGCTCTCAGTGCGTGATCGGCGGGTCAGCCCCACCTGGCTGGGCG
G
GGGATTGTTGAGATGGTGCCGGCGCAGGGGGACGCTGATGGGGTTAGAGC
AGTTGGACGAGGACTGGCTCTTGCTGCGAGGGCGGAACTCATCACTCATG
GCCCGCATGGCCTCCAGGATGGTCTCGTGCATGTTCTGGGCCACCACAGA
GTCATCCACCTGCATCCAGAACTCCCCGGGCCCCGTCACGGCAGAACGGC
CCACCTCGATGAAGAAGAAGTTTTCCGAGTGGCCACAGCGCCTGATGTTC
ATCAGCTGCAGCACCACGGCCGCTGCCTCCGAGTTCAGCTTCACGAAGCT
GATGGTCTTGCTGGTCAGGCAAAGGCGGTAGATACCAATCAGGTTCTTTG
TCTGACCCAGGCCCTTGGGCTTCAGGATCACTTGCCAGACCTCTTTGAAT
GCGGGTCCTGGGGGCACGTCACCGTAGCTCAAGTCCTCCCCAGCCTCACC
AAGGCCGGAGCTGCCGCTGCAGCTGCCCCCACCACCTCCCGCCCCGAGGG
CCGCAGCTCCGTCGTGGTGGCCCTTAGCACGGTTGTGCAGCTGTAGGAGA
GCCTGGTACCAGCTGTCTTGCTCGGCCTCGCTGTCCGCCGCGATGGCAAA
GTGCTCGTCCCGGGTGTAGAGAGCCACCAGGTGCTTGTTCTTGGAGTCAG
CCCGCTTGTTGATGTTGAAGCAGCTCTCAAGGGGGATCGAGCGTTTGGGG
GCGCTCGACTTGTGCCGCCACTTCTTCTCGTTCTCGTAGTACTCGAGGCG
CGCCGGGCCCCCAGCCTCGCTGGCCGCGCGCAGTACGAAGAAGCGTTTGT
GCATGCTCTTGGGTTTGCGCAGGTAGCCCACCTTGCGCACGTCCGAGAAG
CCATCGCTCTCCGGAGGGCTCGCCATGCTGCCACCGCCACCACCAACGCT
GAGCAGAGGGAGGCTCCGAAAAACAACCGGGTGGGGGGCGGAGGCTCCTC
GCCGCGGCCCGGCACATGCAAACAGGGCTGGAGGCAGCAGAAACCCCGAC
TCTGAAATCCACGCCGCCCCCCGCGCCGGGGAGGGGCAGCTGAAGGAGGA
CGCAGCTGCTGAGCCCAGGAGAGAGCCCGACCGGAGTTTTCGGGCGCTTC
ACGCCCGGCGGGGAGGCAGTGCGTCCGGGGTGAGGGCAGCCCCGATCCTC
CGAGAGCCAAGTCTCCTCTCAGCCGCCGCCGCCACCAGGAAGGAGCGAAG
ATGCATCTCTCGTCGCCCCGGCTGGAGTCCGGCACAGGGAGGCGACAGTC
GGGGGTCCCTGCGGTGCCCCTCCAGGAGCGCGCGCGCGCGCGCGCCTTCC
CTCCTGAGTTCCCCTCTGGAAGCAGCGATTCCCGAGGCAAATTAAATATC
CTTGGGCAGGGGGAGGCGGGTTGCCAAGTCCCAACGTTGCACGGGGTTCT
CCCTCCTCCTTCGCCTCCTCCCACCCCCCAACCGTCCCAGCCGCCACCAG
CCGCCCAAATACCAGCTCAGTCGCAGAGACCGCGGCGCTGCGGCTGTTGC
TGTTGCTGCTGCTGCTGCTGCTGCTGCTGCCGCCGCCCGCGGGCGCGTCC
TCTGCAGCCCCCATCCGGGCCCATCTCGGCGGGTGGAGAAAGTGGCTTTT
CCATGCGAAACGATACCGGGCAGCCACTGCAGCTGGGGACCGGCCGGAGG
GACAGACTCATCCCTGCCCCTCGCTCCAGGCGGCTGGGGAGGAGGGGAGG
GGACAAGGGCGAGAGGGGATGGGGGAGGTTTGGGAAGGGTTCGGGGAAGA
CGCCTGTTCCTCGGGAGGCGCTGCCGCTGCAGTTACTTCTCCCCTCCTCC
CTCCTCCTCCTCCTCCTCGGAGAGTTGCCGAGAGCCCCAACCAAAACAAG
CGGCGGCGTCTGGCTCTGCGCGCCGGCCCCCTCCGACCGCGCCGCCGTCT
CACTCGGAGGAGAAAAACACGTGACGGAGCCTCCGCGCTCGGCAGCCGGG
CAGCCGCCGCCGGGAGGCTCCCGCCCGGGTCTCTACATTCCGGGCCGAGC
CCGCCCCGCGCCCGCCCCTCTCCGCCCCCCAGCCGTGCTGCAGCGGCGCC
CGCGCCGACCCCGGCGCAGCGCTAGGACAGCGCCCCTGCGCGGCCGCCAG
CTCCGGCGCAGCGCCTCCCTACACGCGCGCCAACCGGGGCGCCCCCGGCC
CGCAGGAATCCCCGCGGCGGGCGCAGGGGCGGGTGTTTGGGTGGCTACCC
GAAAGCACGAGGGGAGTCTGGGAAAACGGGAGGGGGCGAGCTCGGGAAGA
CGCAGGAGTGAGGGTGGGGGGCCACGGGGGGGCACTGCGGCTACAGAGCC
CCGAGTGGGAGGCCGGGGGGAGGCGGGGGCGCCGGAGCTGGGCTGCCTCT
TACCTATCAGGTAAGACTGACCCACGGGAGGGAGGTTTCTAGTCAAGGCT
GTTTCCCCCAGGAAATTTAAAAGCTAGATTTCGGCTTCAGGGCGATAGGC
CATGGTGAGGGGATCTGTATTTTTTAAGGTTTTTCTAAAGGACTTCTGGG
CCAGCCTTCCACCTTGGGATCCCCAGATTTGCTGCCGTGAGGTACCTGGA
AGGAACAGAGGGACGCCCAAGGGCAGTGACTGGGGGCAGTTATGGTCACT
ACAAAAGGAGAACAGGGATCCCCAGGAAGTGTCTCACGTCCGGCGCTGTG
GAATTAGTTGCTGATCAAGTAGGGAGAAAATGTAAATATCACCTTTTCTC
CTGCTCCCCTTGCCCATCCGTGTGACGGGGGGACTCACACCTAAGGCTGA
GGGCATGGGGGAGGGGAAATGAGAGGTCCCTTATCATATTCCGAAGTCTA
TCCGTGACTTTAAAAAGCTGTTAGGGACCTTCAGAGGGTTCATAAGGCCA
AAACCCTACTGTGCAAATATTGGCTGAAATGTTTGTTTCACACACAGCTC
CCACACCCATTGAACCACCCTATAAGTTTTTAACCTTGACCACAAGGATA
ACAGGGAGAAAACAAGGATGGTGCCAGCTCAACTGTACAGGAAAATGGTT
ACTTCATGAGTGGACCAGATCAGCAGCCTGGGTCTACTTGAAGCGCCCCT
TTCAGGGTCACCTTGCGGTATCAGTCTCCCCACAAACAAACGCTCCCCAG
AGTTTTTTTATTTTTTAAGCCGACCTATAAACAAGCAGTTTGTCCCTGCT
TGCTCGCCTGGCACTAAGGACCCACAGAGATGTCACTCGGTTTAATGCTG
TCTCATTTTGATGCCTGGCCCGTTTGGGGTTGCCGCCCCTTTACTACGCC
GGGACCCTCCTCGGCCTCAGGCTCCCAGGCACTGCAGGATGCTGGCTGGG
CACTCAGCAGTCGAGGCGCTGGGCATGCTCAGTGGCTGGGACCCGGCGGG
CCGCGGGGGAGCGCGCCTCGGCCCAATGCGGCTGAGGGGCGGAGCGAGTT
TCCCGGGGAGGCCTGCTGATTGGTGGGATGGCCTGTTTTTCAAGCCTTGC
ACTGCAGAGGGAAGGTGGGGGTAGAAGGGTTGAGGGTACCAAGGTAGGTT
GCAAAGCGTAGTAAAAAAAGAAAAAGAAGATGATGATGATGAAGGAGAAA
AAGAACCATAGGCGCCATTCAGGGGTGTGCGATACGTAAATTAGGGGGAT
TGGGCATCTGCAGAGGGGCTCCTCCCCACCCAAGCCTGGGAGCCTCTCGT
TTCTCCTCTCCCCTTCCTGTTCCCGGTCGTCATGCAGCCTCATCTAAACC
TGGTTAATCTATAATTTGAGTACACATGTTGAATTGAAAGCCCTCCCTTC
TTTTCCTCTGACAGCTTGGTGATGTGGGCACACGTGCCCACTACAGCAAG
GCTATTAATAGCAGATATTTGGAAATGGAAGCGAGGCGGCACTCACAGTG
ACCGAAATTCTGGGGGTATAGAGTGCCTTACAGGGGGTAGGGCAGGGAGG
TGCAGAATAAAGAAGCCTGAAGTTGGTGGAAAAGTGTAACTGTGTGCCTG
G
Seq. ID. No: 103 IRS1 promoter 2
CCGATTATCCAGATCTGCAGCACTGGCTGCTTCATCCCCAGCCAAGGCT
G
GACTCGTGCCCAAGCCTGTTCCTGGGGTGCAGCGGTGGCCATTGCCACCC
CGAGACAAAATGTAGTGACCGTTGGGGGCGGTCAGGGTGGAGGGCCCCTT
GCCACCCATGCAGATATAGTTGCTTAGCTCCTCCTCACCGCGGGCTGGTG
GGGTGTGGCCCAGGGAATCCGGAGTGACACTGCGGAAGGAACTCCGGAAA
TCGCAGGGACTGGAGCCATACTCATCCGAGGAGATGAAACCGCCATCGCT
GGGGGAACCAGACACCGAAGCACTAGATCGCCGTGGGAAGAGACAATCCG
AGGTGGAGCCATGGCCACTGGTGCTACTGGACGACAGACTGACCGGGCTG
GTGGCCGAAGGCGAGCAGCGGGAAGCCGGCATGGGGATGGAGCGGCTGTG
GTTGAGCGGGGGGTGCAGCCGGGCGCTGCCCCGATGCCGGTGGGCGTGGG
TTCTGTTGGTGCTGGGACTCACAGGGCTGCCGTCCACCGAGGCTGGGCGG
GACATGGTGCCTTCGCCGTCACTGGAGGCGCGGACACGGAAGGAGCCTGG
CTTCCCGCCCACCATGCTGGCCGGGGAGGTGGCGGTGATGCTCTCAGTGC
GTGATCGGCGGGTCAGCCCCACCTGGCTGGGCGGGGGATTGTTGAGATGG
TGCCGGCGCAGGGGGACGCTGATGGGGTTAGAGCAGTTGGACGAGGACTG
GCTCTTGCTGCGAGGGCGGAACTCATCACTCATGGCCCGCATGGCCTCCA
GGATGGTCTCGTGCATGTTCTGGGCCACCACAGAGTCATCCACCTGCATC
CAGAACTCCCCGGGCCCCGTCACGGCAGAACGGCCCACCTCGATGAAGAA
GAAGTTTTCCGAGTGGCCACAGCGCCTGATGTTCATCAGCTGCAGCACCA
CGGCCGCTGCCTCCGAGTTCAGCTTCACGAAGCTGATGGTCTTGCTGGTC
AGGCAAAGGCGGTAGATACCAATCAGGTTCTTTGTCTGACCCAGGCCCTT
GGGCTTCAGGATCACTTGCCAGACCTCTTTGAATGCGGGTCCTGGGGGCA
CGTCACCGTAGCTCAAGTCCTCCCCAGCCTCACCAAGGCCGGAGCTGCCG
CTGCAGCTGCCCCCACCACCTCCCGCCCCGAGGGCCGCAGCTCCGTCGTG
GTGGCCCTTAGCACGGTTGTGCAGCTGTAGGAGAGCCTGGTACCAGCTGT
CTTGCTCGGCCTCGCTGTCCGCCGCGATGGCAAAGTGCTCGTCCCGGGTG
TAGAGAGCCACCAGGTGCTTGTTCTTGGAGTCAGCCCGCTTGTTGATGTT
GAAGCAGCTCTCAAGGGGGATCGAGCGTTTGGGGGCGCTCGACTTGTGCC
GCCACTTCTTCTCGTTCTCGTAGTACTCGAGGCGCGCCGGGCCCCCAGCC
TCGCTGGCCGCGCGCAGTACGAAGAAGCGTTTGTGCATGCTCTTGGGTTT
GCGCAGGTAGCCCACCTTGCGCACGTCCGAGAAGCCATCGCTCTCCGGAG
GGCTCGCCATGCTGCCACCGCCACCACCAACGCTGAGCAGAGGGAGGCTC
CGAAAAACAACCGGGTGGGGGGCGGAGGCTCCTCGCCGCGGCCCGGCACA
TGCAAACAGGGCTGGAGGCAGCAGAAACCCCGACTCTGAAATCCACGCCG
CCCCCCGCGCCGGGGAGGGGCAGCTGAAGGAGGACGCAGCTGCTGAGCCC
AGGAGAGAGCCCGACCGGAGTTTTCGGGCGCTTCACGCCCGGCGGGGAGG
CAGTGCGTCCGGGGTGAGGGCAGCCCCGATCCTCCGAGAGCCAAGTCTCC
TCTCAGCCGCCGCCGCCACCAGGAAGGAGCGAAGATGCATCTCTCGTCGC
CCCGGCTGGAGTCCGGCACAGGGAGGCGACAGTCGGGGGTCCCTGCGGTG
CCCCTCCAGGAGCGCGCGCGCGCGCGCGCCTTCCCTCCTGAGTTCCCCTC
TGGAAGCAGCGATTCCCGAGGCAAATTAAATATCCTTGGGCAGGGGGAGG
CGGGTTGCCAAGTCCCAACGTTGCACGGGGTTCTCCCTCCTCCTTCGCCT
CCTCCCACCCCCCAACCGTCCCAGCCGCCACCAGCCGCCCAAATACCAGC
TCAGTCGCAGAGACCGCGGCGCTGCGGCTGTTGCTGTTGCTGCTGCTGCT
GCTGCTGCTGCTGCCGCCGCCCGCGGGCGCGTCCTCTGCAGCCCCCATCC
GGGCCCATCTCGGCGGGTGGAGAAAGTGGCTTTTCCATGCGAAACGATAC
CGGGCAGCCACTGCAGCTGGGGACCGGCCGGAGGGACAGACTCATCCCTG
CCCCTCGCTCCAGGCGGCTGGGGAGGAGGGGAGGGGACAAGGGCGAGAGG
GGATGGGGGAGGTTTGGGAAGGGTTCGGGGAAGACGCCTGTTCCTCGGGA
GGCGCTGCCGCTGCAGTTACTTCTCCCCTCCTCCCTCCTCCTCCTCCTCC
TCGGAGAGTTGCCGAGAGCCCCAACCAAAACAAGCGGCGGCGTCTGGCTC
TGCGCGCCGGCCCCCTCCGACCGCGCCGCCGTCTCACTCGGAGGAGAAAA
ACACGTGACGGAGCCTCCGCGCTCGGCAGCCGGGCAGCCGCCGCCGGGAG
GCTCCCGCCCGGGTCTCTACATTCCGGGCCGAGCCCGCCCCGCGCCCGCC
CCTCTCCGCCCCCCAGCCGTGCTGCAGCGGCGCCCGCGCCGACCCCGGCG
CAGCGCTAGGACAGCGCCCCTGCGCGGCCGCCAGCTCCGGCGCAGCGCCT
CCCTACACGCGCGCCAACCGGGGCGCCCCCGGCCCGCAGGAATCCCCGCG
GCGGGCGCAGGCGGGGGTGTTTGGGTGGCTACCCGAAAGCACGAGGGGAG
TCTGGGAAAACGGGAGGGGGCGAGCTCGGGAAGACGCAGGAGTGAGGGTG
GGGGGCCACGGGGGGGCACTGCGGCTACAGAGCCCCGAGTGGGAGGCCGG
GGGGAGGCGGGGGCGCCGGAGCTGGGCTGCCTCTTACCTATCAGGTAAGA
CTGACCCACGGGAGGGAGGTTTCTAGTCAAGGCTGTTTCCCCCAGGAAAT
TTAAAAGCTAGATTTCGGCTTCAGGGCGATAGGCCATGGTGAGGGGATCT
GTATTTTTTAAGGTTTTTCTAAAGGACTTCTGGGCCAGCCTTCCACCTTG
GGATCCCCAGATTTGCTGCCGTGAGGTACCTGGAAGGAACAGAGGGACGC
CCAAGGGCAGTGACTGGGGGCAGTTATGGTCACTACAAAAGGAGAACAGG
GATCCCCAGGAAGTGTCTCACGTCCGGCGCTGTGGAATTAGTTGCTGATC
AAGTAGGGAGAAAATGTAAATATCACCTTTTCTCCTGCTCCCCTTGCCCA
TCCGTGTGACGGGGGGACTCACACCTAAGGCTGAGGGCATGGGGGAGGGG
AAATGAGAGGTCCCTTATCATATTCCGAAGTCTATCCGTGACTTTAAAAA
GCTGTTAGGGACCTTCAGAGGGTTCATAAGGCCAAAACCCTACTGTGCAA
ATATTGGCTGAAATGTTTGTTTCACACACAGCTCCCACACCCATTGAACC
ACCCTATAAGTTTTTAACCTTGACCACAAGGATAACAGGGAGAAAACAAG
GATGGTGCCAGCTCAACTGTACAGGAAAATGGTTACTTCATGAGTGGACC
AGATCAGCAGCCTGGGTCTACTTGAAGCGCCCCTTTCAGGGTCACCTTGC
GGTATCAGTCTCCCCACAAACAAACGCTCCCCAGAGTTTTTTTATTTTTT
AAGCCGACCTATAAACAAGCAGTTTGTCCCTGCTTGCTCGCCTGGCACTA
AGGACCCACAGAGATGTCACTCGGTTTAATGCTGTCTCATTTTGATGCCT
GGCCCGTTTGGGGTTGCCGCCCCTTTACTACGCCGGGACCCTCCTCGGCC
TCAGGCTCCCAGGCACTGCAGGATGCTGGCTGGGCACTCAGCAGTCGAGG
C
Seq. ID. No: 104DGAT1 promoter
GAGTTCAGGAGTTCGAGACCAGCCTGACCAACATGGAGAAACCCCATCT
C
TACTAAAAATACAAAATTAGCCGGGTGTGGTGGGCGCCTCTAATCCCAGC
TACTCTGGAGGCTGAGGCAGGAGAATCACTTGAACCTGGGAGGCAGAGGT
TGCAGTGAGCCAAGAATGTGCCATTTTACTCCAGCCTGGGCAACAAGAGC
AAAACTAGTCTCAAAAAAAAAGAAAAGAAAAAAAAAGAAATCTCAATTCC
CACGTCCTCCACAGGTCATGTAGCCATCCCCCTGCCTCCTGGAGAGAGGG
TAACTCCAGGAGCCACACCTCCTTTTCAGGTAGGAGGTGCCTAGGCCAGC
CTCCCTCACCAGCACCCCAGGCCCACAGCAGGCAAGCACCCCTCTCAGGA
GGGCCACCCTCCTCCCCACGGCCCCTTCAAGGCCTCCGTGCCCGACCTTT
GTCCATGTTGCTCCATGCCTGACTCTCGCTGAGGTTATCTTATCTCCCAG
ATGAACTGAGCACCCTGAGGGCCACGACAGTGCCTGCCCTCCCCCAACCC
CCTCCCCATGGTGCCCCGTGAAGGAAAGCTCTGTTCTTGCTCACTGAGAG
AGCAAATCATTGCCAACTACATGTCCAAAATCAGCCCACTTCTGCCTTGA
TAACCTCTAGGCCCTCAGCTGCTCTCTAAAACAAAGCCCTGAGGGCCCCT
GGGCCAGGGTAAAGGCCATAAAGGGGGGATGGAGGGTGGCAAGGGGATTG
CTCAAGGCCAGCTTGGTGGAGGAAGCCAATGCCCAGGCTGGCCAAGTCCT
TCCCATCGGCCATGGTTCTCTCCCTCCACCCCTCTCTGTCAGTCATCCAG
CTGAGCAGGGCACACGCCTGCAGGTGTGTCCACCACCCCCATACCCCACT
CCCCACCTGGAGGATGGAAGTCCCAACACAGAACCCAACTCTCTGTCCCT
ACAACAGACGCTGGCTCTGGCAACATCGGCAGGGCTCCAGGCCAGCCTAA
CCACTGGAGCAATGAATGGGCCACGCAGCCACTCTCAGAAGGGTCTGCAA
GGACAAGAACGCCTCTAACACTGCCCCTTGTCCGGGGCAGCTGCTGATTC
CCCTGTCCTCGGACAGGCCTGGGCTCCTCTGGCCCCCTCCCTGGCAGGCC
AGCCCTCCTCTGGGAGCCTTTCCTACCCGCAAGGCACCGCTCATAGCACC
AAGAAGGAACCTATCACCACAAACTGCCCAGATCTTATCTGACTCCTTCC
ATATAAGGGGACACTGTCCAGGGCCGGGCCCCCACCAGCCCGCCCCTCGA
AAAGCTCCTCAGGAGCCCCTGAGGATGCACAGACCCAAGAGGATCTGGAT
GCGGGACACCTGCCGGCTCCCCTCTGCCTGGCCAAGGCCAGCTCCAGAGC
CCCTACCCAGTGACCTTGTCAGGTGACTCTCCCCAAGCCTCAGTTTCCCC
ACAGGGCCCATAGGGCACACCGATCCCCGCTTAGCCCAGGGCCCATGTTC
TCGGACTCGGAGCTCGCGCTTCGGCCAACCCCGGAAAGTCGCGGGGCCCT
TGGGTCAGAGGTTAGGGGGTCAGGCGCTCCGCTACCTCAGCTCCCAGTGG
CCGCTGCCCACGCCGGCGTCTCCGTCCTTGTTGGGGGCCGGGGCTGGCGC
GTCCCCCGCGGCTCCCACGTCGGGGCCCGCAGCGGCGTCCCGCACCTCCT
CTTCCGCCGCCGCAGGCCCGCCGCCGCCGTGGCTCGAGGGCCGCGACCCT
GTCCTCCGGCGCCGGGAGCTGCCGCGGTCGCCCATGGCCTCAGCCCGCAC
CCGGCCGCAGCCAAGCGTGGGCCCGCCGGGTTCGTAGCGCCCGAGGCGCG
CGGCCCCACCTCCGGGCCCTAGACAACGGCCGCCACTGCCCCCTGCCGGC
CGCCGTAGCCCGGGTGACCGCCTCACCAGCGCGTTCAACCCGCCCGCGTC
GGGCCCGTCGGCCTCAAGGACAACGGCTGCGTTGCTCCGGAGCCGCTAAC
TAATGGACGGCCGCCTCTCTCGTCCATTTGTAGTCCGCGGCCGACCTGAG
GCGGGGCCTGGAGAGGCTGACGGGGCGCAGGGCACCCTGGGAGTCGTAGT
CGCCGCGGGCGCGCCGGCGCAGAGCACCCTGGGAGTCGTAGTCGCCGCGG
CCGCACCGGCGCAGAGCACCCTGGGAGCCGTGGCAGCTCCCAGGGCCGGG
GCTGCGGGCCGCAAGTGCGCGCAGCGGGAACGGACCTTGCATCGCTCCCG
CGTGGTCCCGGGGTCCTGTCCGGCCCCACGGAGCGGGATGGTCCGCTGAG
GGGTGCGCTGGGCCCCGACTTCCTGGCCGCTGTGCGCCCGCCCCTCGTCC
CACTTGTGTACACACGGGGTCTCGGCGAGTCACTCCGGGCAATCATTTAG
GAAGCGCCTAGTGCGCCACGGTCCAGCTGTCCGGGCCGGTGTCCGCGGCG
GCTCGCGTCGCCCGCCACCCAGCGCGTTGGTTTCTGCGCACGCAGCTTAC
AGTAACGTCCCTGGCGCGCTTCTGCTCCGCGAGCCCTCTGGTTACCCCGG
CAGGAACCTGCTTTTCTTCGCCTTAGGGGGACGTCAGACAAGGGCCAGGT
CAGAAGACCAAGCTGGAGAAATGCACCAGGCCCTGCGTCACCGGGACGCC
TGCCTTGCACGCGACTCCCAGGCTCTCCACCTCCCCGCCTGCAGAGCCCG
GCGAGAGGAGGGCGAGGGCGAGGGCGAGGGCGCAGGCCCCTTGGTGGTGT
CTTCTCAGCCCACCCACTCTCTCCACAAGGAGCTCCACGTCCCTCAACTC
TCAGGGCCTCCCAGTTTCCAACGTGGGCCAGCCTCTCCAGGATGTCAGAA
GTCTGGGCCGCTGGATCGCCGGGACTCAAGAGGCTTCTGTGCATTTGCAC
GGCCTGTGCAACCGCCCTGTTGTTTTTTGTTTTCTTTTTTCTTTTTTTGA
GACAGTCTCACTCTGTCGCCAGGCTGGAGTGCAGTAGCGCGATCTCGTCT
CACTGCAACCTCCACCTCCTGGGTTCAAGCGATTCTCCTGCCTCAGCCTC
CCGAGTAGCCAGGACTACAGGCGCGTGCCACCACGCCCAGCTAATTTTTC
TGTTTTTAGTAGAGACGGGCTTTCACCATATTGGCCAGGCTGGTCTCGAT
CTCCTGACCTCGTGATCCGCCCACCTCGGCCTCCCAAAGTGCTGGGATTA
TAGGCGCAAGCCACCGCGCCTGGCCTGTTGTTATTTTTTCTAAAGATGGG
GGTCTCGCTATTCTGCCCAGGCTGGTCTCGAACTCCTGGCCTCACGCAAT
CCTCCCACCTCGGCCTCTGAAGGTGCTGGGATTACAGGTATGAGCCACTG
CCCCAGACATCTTGTATGTTAAATGAAGCAACTAATCAACTGTAACAGCC
ACAGTTGCGGCCCACCTTGGCTTGGTGTGCATATTGCACAGGGCATGGAG
TGAGAACATCCTGGATCTCAGGATGCCAGGGTTCACTGAGGACTCTGACT
CTAGAAACACCTTCATTCCTCTGGGGTGAGCAGGGGCAAGCAGCACTTCT
CTGGAAGCAAACAGGTGCTCCTCCTGAGGTGGGGTCCTCTCCCGTGGGCT
GGTCACAGCCTCGCCTCTGAGCTCCGTCCTCTTCCACCACCTCCACCAAC
AGTGGCCAGCAGGGCTGCCCCCTTGGTTGCCCCCCTGGTGACATATTCTT
GTCACCACAGGCCGCTGTGGGTTGCTGCCAACCAGGTATCAGGCAGGCTG
GAGCTACCTGGCAGATGCCCTTTGTGTAGCCTGCCGAGTGGGGCCGCCCT
CACTGGACCCAAGAGCCCCTCACACATGGGCCTTGGTCGGTCAGGACCAG
CCCCATCTCCTACCCAGTCTCTCAGAATATTCATTTGTTGAATGCAGTCG
CTGGCAGACACACTCTGTGGCCAGGTAGGCCCAGACCTGTCTGAGTATGG
ACTGACAGCAAGCTCAGAGAGGTCTGGACCTGGCCACGGAGCACCTCATG
C
Seq. ID. No: 105 DGAT2 promoter 1
ACGAGCCACAGACTGACCTGGGATCTTCCAGGCCAGCAGGAGTCTTGCC
T
CCAAGAGCACCCTCTCTGAGGAGAGGATGCCAGGATTTACTGGCACCTTC
AGTATCCTCCTGGCTTCACTCCTCTTTGCCCAAAAACAAACCACACCTGT
TTCTACCTCCCAGCCTTTGCACTTACCAGTCCCCATGTCTCCCCCCATCC
CCCACATGTGGCATGCCACTAGCCTACCCTCAGTTTCCTAAAAGGCACTA
AGATCTTTCCAGCCTCATGGCCTCACCACATGCTGGGAGCTCTGCCTGGA
ATGCTTTTCTTTCTACTCTTGGCCAATTCGGGCCTTGGGGCCCAGTAACA
CGGTTGAGAGATTTACTCCTGTGGCCTTGAGAGGGCTGAACAAATGGAAG
CATCTCTAGGTTGCACCAGAGGCATCTATTACCTGCTGCCCTGTGCCTGT
AACTTCTGTCATTCTCCTCTCCCCCTGTGAGAACATGATAAAGACCACAA
AGGCAGGGAATTCCTCTCCTCTTATGCTGTGACCCCACAGCTATCTCGCT
CCAGCTATGCCTAAGAAATCTGTCTTATCACTGACATGCTTGTTGTCCCC
ACTCGTTCCCTGAGCCTCCATCCAACAAGTCGCTCCACCACATGCTGGTG
GATCTCGCTTCCTATGCTCTTCCCTAGATCAGGCCTTCCCTCCATTCCCT
CTACCACTGCCGTGCCTTGAGGCTCATCCTCTCTCACATGGATCCCCCGC
CCCTACAGCCTCCCCACTGCCCTCCTGACCTACAGCCTCTCCTGTCCATT
TCCCATACCATGGCTAAGGACACTTAAAACCCACCTGACCAGCCATTTCC
CTACTAAAGCTCTCCCAAGACCCGGCTCCCCCCCAATATCTCAAGAGTGG
CACTCCCAAACCTCTCTCAGTTCAAGCACCCGCGCCTCTCACTTCAGCCT
CATTTTCCACAGTTGCCTGGGCTCCAGCCACTCCAGGGCCCCCAGCCCTG
CCCCAAACATGCTGGCTTTTCTAACACTTTATGCCTATCTTTCTGCTTTG
CCCTCACACCTTCCATCTGCAAAATTCCTATTCATTAATGGGCAAACCAT
TCTGGAATGCTTTCTATGTGCCAGACACTACCTAGGCTCTTTTTATACCT
TATCTCATTTACTTCTCAAAGTACCTCACAAAAATAATAATTATTATTCC
CATTTTACAGATATGGAAACTGAGGCTCAAGAGAGGCAGGGCAAGAACTG
GACTCCTAGTCTATCTGATTCCAAACCTGCTGTGCACCAACCTCTCTGGG
AAGTGCACCAGCGCCCCCACTCCCCCAGCCCAGGGCAGAGTCTTCTCTAC
TCTGTGGTTCCATAGCCCCCTGGTTTCTCGCTTGTCCCGGCCGTTGTCAC
CAGTGACTGTGTGCGGCTGTCTCTGCTGCCCCCCAGCGTGAGCATCCCAA
GGGCAGAACCTGGGCTGATCTGGCTGGGTCCCCAGCACCCAGCAGGGTAT
AGGTGCTCTGTGAGGTTTCTTAATAAAGAGATGGAAAGCCAGAAGCAGTT
TGGGTGGTTGAGATACCCAGCCCTAACCTAACTAATCCTGATACCAGTGA
CCAAAAGCGGGACCTTCGCATCTTTGCTGCCAAAAGACAGCCCCTCCTAA
AGAGTAAAGGCCCGACCCCCTGCACGCCCTCTGCCTGCACCGCACGTGCT
TGGTTTTCCCCGCCCGGGTACTGGCCGCCGGGCCGTACCAATCTCCGCGG
GGGAGCGCCCGGGGTCGGACTGAGGGAGCGAGGGGAATAACCGGGCGCGC
CCCTTGGAAGCAGGGCTCAGAGCTGCTCTCCTCTCACGCATTCCCCGGAT
CCGCGCGGAGCAGGCTGCTGGCCAGCCCCGGGCCCGCGCCAAGCAGAGCC
TCAGGTGCGGTTCCCCCACAAGCAAGTGGCGCGGGCGGCGGCTTAGAACG
GCCCGCCCCGCCCGCCGCGTCGGCGCCTGCCCCGTTGTGAGGTGATAAAG
TGTTGCGCTCCGGGACGCCAGCGCCGCGGCTGCCGCCTCTGCTGGGGTCT
AGGCTGTTTCTCTCGCGCCACCACTGGCCGCCGGCCGCAGCTCCAGGTGT
CCTAGCCGCCCAGCCTCGACGCCGTCCCGGGACCCCTGTGCTCTGCGCGA
AGCCCTGGCCCCGGGGGCCGGGGCATGGGCCAGGGGCGCGGGGTGAAGCG
GCTTCCCGCGGGGCCGTGACTGGGCGGGCTTCAGCCATGAAGACCCTCAT
AGCCGCCTACTCCGGGGTCCTGCGCGGCGAGCGTCAGGCCGAGGCTGACC
GGAGCCAGCGCTCTCACGGAGGACCTGCGCTGTCGCGCGAGGGGTCTGGG
AGATGGGGTGAGTGCCACGGCGCAGGGGTTATGGACCTGCGAGAAGATTT
TCTGGAAAGGGCCCTGTGGCAGGCTGGTGGGTACTGATGAGTCCACGTTC
ATTCTCCACTGTGGCACTCATCAATTTTTACGACCTCTGTTACATCGCTT
TCCACCCGCCCCCCAGCTTGTTTCCCTCATCCGTGAGGTGGGAGCGGTAC
CACCCACCATTCTTAGTTATTAGGGATATTCGAGAACTCCTCCCCAGCCC
CCACTGCGGCTGGTGACCCCTGGCACTTCCCTCCCCTCTCCCTTCACCAG
GTAGAGCGAGCTTTGGCAGTGATAGACTGGATGGGCAGGATGGTATGGGT
TTTGCCGTCTCCTGAGACAGCCACCAGACGGGGAACATGCCCGACTGGAA
CAGGTGTGTCTGCCCTGTCCTCTGTCCCACATCCATCCTCTCGCCCAGGC
TCTGGAGCCCACATAGCAGCAACTCTTGAGCCTGGCATGCTTAGAAGGCA
GCGGAGAGGACCCTGCATGTCCTCCAAGGTAGAACTGAGGTCCTCAGTGA
ATCGCGCAGAGTTGAAATCAACCCCCGCCCCGCACCCCCCGCAGCTTTTC
CCAAGCGAGGAATAGCAACTCTTCCAACCCCCACCTCCCTTACCTAGAGC
TGGAGAAACTGAAGTGGGAGGAAGCATGCCTAAGTTTTCCTTAGCTGATG
CCTTGACCCCTGGATTCAAGTACAAATCCGGTAGACCCTGGGAAGTCATC
ACAGCTGTCCTGGTCTGCGTGTGTGTCTTGCATGAAGCCCCTCTCCCTCT
TTCTAAGTCTGTATTCTGTTTGCTGAGCCTCTCTGACATGGATTTTTCTT
TAGTAACTAACGGTCGCCTACACCGCCCACCTTTGTTACAAAAATAAAGC
TTGCTAATTATGGAATTTTTGAAAATATAGACAAATGAAAAAAATTTCTG
GTAAATCGACTATCCAAAGATACCTTCCTATTAGCATTTTAGTATATCTC
TTGATCATTTTCTTTATACACATTGCATAGATACAATTGAGGACATGCTG
TAGATATAGTTATGAATCTTGTTTTCTCCCCCTTAATGTAACATCGAGGT
TTCCCTTCTGTTAATCAGAATCACTTACAATGTCCTAGTGGTTGCAATAA
CCCACCCTGCAGCTGTACCACACTTTAACTGTGACCTAGGCATTGGCATT
GCTTCTTGTGTGATTATCGCTGTGGTTATCTGCCCCTCTTGGTGTGGGTG
CTGCTCGTAGCCCTTGAAGAGGAACCCAGCTGCTGCCCTGTCTCGGGGGC
GAGCAGCTTGAGCTGCCCCATGTATCCAGCCAGTAGCCTCTGACAGCCCC
TTCTCTCACTTGAGTCCTTTTCTGTTCCCTGTGTCCTTTGATGTCCTTAG
GGACATCAATGGATGAATGGACTTGCCCTTGTTCATGCTGTTAAAAATGT
TTTTGCACTGGGCAGTGGGATAGGGATGTTCTCTGTGGTAGTGCTTCCTG
GAGACCCCATTCCGTCGGCTCTGCCATCCACAGGCTGGGAGCTGTGTCTT
CCAGGAGGCAGTGACCCTGGCTGTCATGTTTTTGACTTAGAGTTTGTTCC
TTAGGAGAACTTGTACTCTAGCGAATGGTTTTAACCAAGCCACTTAATAT
C
Seq. ID. No: 106 DGAT2 promoter 2
CCATCCCCCACATGTGGCATGCCACTAGCCTACCCTCAGTTTCCTAAAAG
GCACTAAGATCTTTCCAGCCTCATGGCCTCACCACATGCTGGGAGCTCTG
CCTGGAATGCTTTTCTTTCTACTCTTGGCCAATTCGGGCCTTGGGGCCCA
GTAACACGGTTGAGAGATTTACTCCTGTGGCCTTGAGAGGGCTGAACAAA
TGGAAGCATCTCTAGGTTGCACCAGAGGCATCTATTACCTGCTGCCCTGT
GCCTGTAACTTCTGTCATTCTCCTCTCCCCCTGTGAGAACATGATAAAGA
CCACAAAGGCAGGGAATTCCTCTCCTCTTATGCTGTGACCCCACAGCTAT
CTCGCTCCAGCTATGCCTAAGAAATCTGTCTTATCACTGACATGCTTGTT
GTCCCCACTCGTTCCCTGAGCCTCCATCCAACAAGTCGCTCCACCACATG
CTGGTGGATCTCGCTTCCTATGCTCTTCCCTAGATCAGGCCTTCCCTCCA
TTCCCTCTACCACTGCCGTGCCTTGAGGCTCATCCTCTCTCACATGGATC
CCCCGCCCCTACAGCCTCCCCACTGCCCTCCTGACCTACAGCCTCTCCTG
TCCATTTCCCATACCATGGCTAAGGACACTTAAAACCCACCTGACCAGCC
ATTTCCCTACTAAAGCTCTCCCAAGACCCGGCTCCCCCCCAATATCTCAA
GAGTGGCACTCCCAAACCTCTCTCAGTTCAAGCACCCGCGCCTCTCACTT
CAGCCTCATTTTCCACAGTTGCCTGGGCTCCAGCCACTCCAGGGCCCCCA
GCCCTGCCCCAAACATGCTGGCTTTTCTAACACTTTATGCCTATCTTTCT
GCTTTGCCCTCACACCTTCCATCTGCAAAATTCCTATTCATTAATGGGCA
AACCATTCTGGAATGCTTTCTATGTGCCAGACACTACCTAGGCTCTTTTT
ATACCTTATCTCATTTACTTCTCAAAGTACCTCACAAAAATAATAATTAT
TATTCCCATTTTACAGATATGGAAACTGAGGCTCAAGAGAGGCAGGGCAA
GAACTGGACTCCTAGTCTATCTGATTCCAAACCTGCTGTGCACCAACCTC
TCTGGGAAGTGCACCAGCGCCCCCACTCCCCCAGCCCAGGGCAGAGTCTT
CTCTACTCTGTGGTTCCATAGCCCCCTGGTTTCTCGCTTGTCCCGGCCGT
TGTCACCAGTGACTGTGTGCGGCTGTCTCTGCTGCCCCCCAGCGTGAGCA
TCCCAAGGGCAGAACCTGGGCTGATCTGGCTGGGTCCCCAGCACCCAGCA
GGGTATAGGTGCTCTGTGAGGTTTCTTAATAAAGAGATGGAAAGCCAGAA
GCAGTTTGGGTGGTTGAGATACCCAGCCCTAACCTAACTAATCCTGATAC
CAGTGACCAAAAGCGGGACCTTCGCATCTTTGCTGCCAAAAGACAGCCCC
TCCTAAAGAGTAAAGGCCCGACCCCCTGCACGCCCTCTGCCTGCACCGCA
CGTGCTTGGTTTTCCCCGCCCGGGTACTGGCCGCCGGGCCGTACCAATCT
CCGCGGGGGAGCGCCCGGGGTCGGACTGAGGGAGCGAGGGGAATAACCGG
GCGCGCCCCTTGGAAGCAGGGCTCAGAGCTGCTCTCCTCTCACGCATTCC
CCGGATCCGCGCGGAGCAGGCTGCTGGCCAGCCCCGGGCCCGCGCCAAGC
AGAGCCTCAGGTGCGGTTCCCCCACAAGCAAGTGGCGCGGGCGGCGGCTT
AGAACGGCCCGCCCCGCCCGCCGCGTCGGCGCCTGCCCCGTTGTGAGGTG
ATAAAGTGTTGCGCTCCGGGACGCCAGCGCCGCGGCTGCCGCCTCTGCTG
GGGTCTAGGCTGTTTCTCTCGCGCCACCACTGGCCGCCGGCCGCAGCTCC
AGGTGTCCTAGCCGCCCAGCCTCGACGCCGTCCCGGGACCCCTGTGCTCT
GCGCGAAGCCCTGGCCCCGGGGGCCGGGGCATGGGCCAGGGGCGCGGGGT
GAAGCGGCTTCCCGCGGGGCCGTGACTGGGCGGGCTTCAGCCATGAAGAC
CCTCATAGCCGCCTACTCCGGGGTCCTGCGCGGCGAGCGTCAGGCCGAGG
CTGACCGGAGCCAGCGCTCTCACGGAGGACCTGCGCTGTCGCGCGAGGGG
TCTGGGAGATGGGGTGAGTGCCACGGCGCAGGGGTTATGGACCTGCGAGA
AGATTTTCTGGAAAGGGCCCTGTGGCAGGCTGGTGGGTACTGATGAGTCC
ACGTTCATTCTCCACTGTGGCACTCATCAATTTTTACGACCTCTGTTACA
TCGCTTTCCACCCGCCCCCCAGCTTGTTTCCCTCATCCGTGAGGTGGGAG
CGGTACCACCCACCATTCTTAGTTATTAGGGATATTCGAGAACTCCTCCC
CAGCCCCCACTGCGGCTGGTGACCCCTGGCACTTCCCTCCCCTCTCCCTT
CACCAGGTAGAGCGAGCTTTGGCAGTGATAGACTGGATGGGCAGGATGGT
ATGGGTTTTGCCGTCTCCTGAGACAGCCACCAGACGGGGAACATGCCCGA
CTGGAACAGGTGTGTCTGCCCTGTCCTCTGTCCCACATCCATCCTCTCGC
CCAGGCTCTGGAGCCCACATAGCAGCAACTCTTGAGCCTGGCATGCTTAG
AAGGCAGCGGAGAGGACCCTGCATGTCCTCCAAGGTAGAACTGAGGTCCT
CAGTGAATCGCGCAGAGTTGAAATCAACCCCCGCCCCGCACCCCCCGCAG
CTTTTCCCAAGCGAGGAATAGCAACTCTTCCAACCCCCACCTCCCTTACC
TAGAGCTGGAGAAACTGAAGTGGGAGGAAGCATGCCTAAGTTTTCCTTAG
CTGATGCCTTGACCCCTGGATTCAAGTACAAATCCGGTAGACCCTGGGAA
GTCATCACAGCTGTCCTGGTCTGCGTGTGTGTCTTGCATGAAGCCCCTCT
CCCTCTTTCTAAGTCTGTATTCTGTTTGCTGAGCCTCTCTGACATGGATT
TTTCTTTAGTAACTAACGGTCGCCTACACCGCCCACCTTTGTTACAAAAA
TAAAGCTTGCTAATTATGGAATTTTTGAAAATATAGACAAATGAAAAAAA
TTTCTGGTAAATCGACTATCCAAAGATACCTTCCTATTAGCATTTTAGTA
TATCTCTTGATCATTTTCTTTATACACATTGCATAGATACAATTGAGGAC
ATGCTGTAGATATAGTTATGAATCTTGTTTTCTCCCCCTTAATGTAACAT
CGAGGTTTCCCTTCTGTTAATCAGAATCACTTACAATGTCCTAGTGGTTG
CAATAACCCACCCTGCAGCTGTACCACACTTTAACTGTGACCTAGGCATT
GGCATTGCTTCTTGTGTGATTATCGCTGTGGTTATCTGCCCCTCTTGGTG
TGGGTGCTGCTCGTAGCCCTTGAAGAGGAACCCAGCTGCTGCCCTGTCTC
GGGGGCGAGCAGCTTGAGCTGCCCCATGTATCCAGCCAGTAGCCTCTGAC
AGCCCCTTCTCTCACTTGAGTCCTTTTCTGTTCCCTGTGTCCTTTGATGT
CCTTAGGGACATCAATGGATGAATGGACTTGCCCTTGTTCATGCTGTTAA
AAATGTTTTTGCACTGGGCAGTGGGATAGGGATGTTCTCTGTGGTAGTGC
TTCCTGGAGACCCCATTCCGTCGGCTCTGCCATCCACAGGCTGGGAGCTG
TGTCTTCCAGGAGGCAGTGACCCTGGCTGTCATGTTTTTGACTTAGAGTT
TGTTCCTTAGGAGAACTTGTACTCTAGCGAATGGTTTTAACCAAGCCACT
TAATATCATGTCAGGAACATTTCCCCATGTTGTTATCAGATCTTGAAAAC
TTTTTTTTTTTTAAACTGGGTGCAAGGATTTACATCATGGAATGTAGGAA
GGGCTGGTATGAAATGCAAACCAGTCAGTTCAGCTTTCTGGGATCTACTT
TGGTGAAAGATTGGGTGGAGTAGGGGAGGGCACTGAAGCACATTTTGTTA
T
Seq. ID. No: 107 ACACA promoter 1
AACTATAAATTCACTAAGATAAATGCCACCATTTCCCAAGACTTCCTTTT
TAAAAAGCAATTTAATCTTATGCTTGATGTATTTCTATTATGCACTGGTA
AATCAAAACAAAACAAGCCTGGCGTGGTGGCTCATGCCTATAATCCCAGC
ACTTTGGGAGGCCGAGGAGGGTGGATCACCTGAGGTCAGGAGTTGGAGAC
CAGCCTGGCCAACATGGTGAAACCCTGTCTCTACCGAAAATACAAAAATT
AGCCAGGCGTAGTGGCGCACGCGTGTAATCCCAGCTACTCGGGAGGCTGA
GGCATGAGAATCTCTTGAAACCAGGAGACGAAGGTTGCAGTGAGCCAAGA
TCGCACCATTGCACTCCAGTCTGGGTGACAGAGCAAGACTCCATCTCAAA
CAAACAAACAAAACAACAACAACAAAATTCTACAACACTAGTAAACACAG
ATGGCTTCATAAATTAATACTTTACCTTTGGCATCTTTCATATGCCCACT
TTAAAATGTTTAATATTCTTGGGTTCACATCAGTCCTCCTGTTATGCATT
CAGTTTTACTGAGAAATGAAGAAACTGAGGCAAGAGAAGTGGGAAAAAAC
TTGCTATTTGTAGCAAGCAGGCATCTATCTGGACAGGTAAACTGTGGTCC
TGCTCTATTAGGCCACAATGCTTTTCTGAAAAGTGCCTGTCAAATAAGCA
CCACAGAAATCCTTCTCATCACAGACATCCAGGGCATCCCCCTCAAGAAG
GGACAAATATGAGTATTCTTCCCTGCTGAGTGAGAAAAGACCATGTAACC
AAAGAAAGCATGCTTCTCAATCTATACTACCGAATTGGGAGGGAAAAAAT
GAATGGAAAGAGAGAGAAAATCCAAGGAAATAAACTTCTGAGACTGAAGA
TGTGGTTATTAGGTTTATCCACACATTAGGAAATTACTGAAACATCGCTT
AAGTAGAAACAGAATCAGGAAGGCCTAGGGTCTTTTTTTTTATTCCAGTC
TTAAACCTCCAAACAAAAATATCTGAAATTAAACTATTTCTCCTGAGCAG
TTATATTCTTAAATGTCTCTATAGAACCCACAAGGCCTACACGAAGTCCC
TGATTTTTAGAGGTGATTTCAAGTGTCCATTAGTGTCCACTCTCCCACTC
TCAAATCCAAAATAATGTTGGCCCCGATCCGGGCAGGACCTTCACGCACA
TTTTCCTGTGAAATCATTAGCTTCACATCAGCTTCCTACGTCCTATTTCC
AGTAACTTCCCTTGATCAGCATGGCAAACGAGGAGAGTAACAAAAGGCAT
GTACATTTACTATGTCCCTCGCAGAGAACATAATAAAATCCTGGGAACAG
GAACTGGCAGATACAACCTGCCTGGCCCTGGCTCTCGCCACGATACAAAG
GCACGGAGAGAGCAAGTGAGGTCTGCACCCCACACCTCACTCTGCAAGAG
AGAAAGGACTTTCAATGCAATCAGACCCAACCCGCAACGCCCCCTTCCCA
CGTGGTCTTCTCCCTCTCCCGAGCCCAGAATCGGATTGGAGAGGGGTCTG
GAAGGAATCAGACCACGCTGGAGCTGGGCAAGAGGGGTGGCGGGCGAGCT
CCAGGCAGAAACTCCAGCCCCCCTTCCTAGAGGAGCCCTGAACTGTACGC
CTCCCAGTTCCGGGATCTGAATCCCCACCTCCCGAGACCCCAGACCTCTC
CATGTCTCTAGGGAGGGGACCTCAAGGCCTCAGAGCTGCGGGAAGGGCTG
GGCTGCCCCGCACCCGATCTGGATAGTGTGGAGCCCAGGCCGCGTGCGGG
TGAGCGGAGGCTGGGAGGCGGCCCCCTGCCTGCCGCCGCCCCTCCCTTGG
CCCCAGTTTCTCCAGGTCCCCGGTCACAGGTTCTGGGCCGGCCCTCACCT
CACCTCAGGGTGGCAACGTGTGCGGTCGGGCGATTCCGGAGCCCCTGCGT
GGAGGAACTGCTGGGCGGGAGGAGACGCCGGCGGCTCGGGCGATGGCTGA
CAGGCGTGCAGCACCAGGCCGGCCCGGCACACGGAGAAGGGGCGGGGCTT
CAGGGGCCTGACCCGCCCCCGCCCCGCCCCTGTCTCCCACCTCAGCCGGC
GAGCTCCATGAGGTGACTACGTCCGGGGTTACTCCAGGCCGTTCACCTCC
GACCCCGCTCCGGGCTGCGGCGCTGCCAGGGCCGCCGGGGGGCGAGGCGC
GGGCCCGAGGGCTCAGCGGCAGCCAATGGGAGGGCGGCGCGGGGCGGAGC
GATGTGAGGCGATGCCGATGGTCCGAGCGCGGATCTGCCATTCGGGGCCC
AGCGGCTAGAGGGCGGGCGGGCCGCGGGCGGGCCGCGGGCGGGCCCTGGG
CAGTCGTGGCGCCCCTGGCCCGGCCTTCGCGGCCCCTTCTGCCACAGCCG
GGTACGGGGCCAACCCCTCGGCTCCTCCTCCTGGCGGAGAGGGGAGTGGG
GGAAGCGGTCCCGCATGCGCCTCGCATCAGGGACAGAGGCGTCCCAAGTG
AGGGGCCAGGACTAGCCGGCCTGTAGAGGCGGGGAAAGGGAACCCAGAAT
TAAGGCGGGCTAGAATGGGGCAGGCGGTGTGCGGGCCCGTTTGTCTGTTT
AATCCTAACACATTAACAAGGGGTGGGCAATCGCGTCCACTACCTGTCCT
CATCACGCTGGCTGGTTTCCAAGTCTGGGCGAGCTAAGTAACCCCATGCT
GGAACTCCATAACCAGCCCCCCGTTTCGCTCCTCAGCCCCGAGACAGCCC
ACTCCAAAGGTGTTATACATGCTGGATCCTGGCGTGTTGAGGAGAAGCCT
CAGTAAACGAAGCTCAGTAAGAAGGGGAATGTACCAAAGAACCACCGTCT
TCTGCCTGCCCTGAGCAAAATAGTTGCCACAGCGTTCCAGACTTTTCGGA
TTGGAGAATGCTTCCCGTCCTCCACTGTGGGCACGCCGAGCCTTTGAGAG
GAAGCCAGTCGGTGTCTCTAGTTTTGCCAGTTACTTCATAGCAAGACTTT
CATGGTAGCTGTGTGTTGAAAATTAATAATAACTGGAAGGGGTCCCAACA
CAATACAAACATTGCGTAGGGAAGAAACTTGGAGAAAATTGCACTTGAGA
AGAACAGGGTGAACTCAGCCCTTCCAAGGAGGAATTTAATGAAGCATGCA
GCGCACCTTCTTAATGCTTACAAACTTGTGAGGAAGGCCAGGAGAGGTGC
AGAGGATGACAGTTTGGTAGGAAGAAGTTCCACGCCAGAGAGTAAAAAGT
GAATAGGATAGAAATACATGCCCAGGTAAGAAGTTCTTGGCGGAAGCTGA
GGCAGAAAGAGGGAAAGATTGGTCAACAAGGAACAATATTTATCTAGGAG
CCCTCATAATCCCACTAAGTGGGGCTTCCCTAAGAATTTATCAACCCATT
TTTAAAATAATAGGGTCAAGCACAGTGGCACGTACCTGGACTGTAGTCCC
ACCTACTCAGAAGGCTGAGGTGGGTGGATGGCTTGAGGCGAGGAGTTCAA
GTCTATATGTAGTGTACTGTGCCTGCGCCTGTGAATAGCCACTGCACCCC
AGCACTGGCAACGTAAAAAGACCCCATCTCTAAATAAATAAGTTAATTAA
ATAACAGGGATTGGAAGCAGAGAACTTAGGAAAGATGTGGACTTTACAGA
ACAGGGATTCATATTTCACAATAATTATATCACTAATATATCAATCAGTT
GGACAAATGTAGTGGGAGGAAAAAGATAATTGGGCAAGACTTCCTGGTTT
AGTAAGATGTTTGATTTGACTCACCGAAAAAAATGGCCTGGGAAGAGGGA
AGCCTTGCCTTTCCATAACAGCAGTCTCTGAAGCTGACATGAAGGGAGTG
ACTTCAAAAGAACTAAAAACCATGCGGTTGCCTCTTACTTTTCTTTTACC
TTTCCTAGATCCTCTTAAGTAATAGGAATTCTTTCCCCAGGATTCTTTTT
TTTTTTTTTTTGAGATGGAGTCTCGCTCTGTCGCCCAGGCTGGAGTGCCG
T
Seq. ID. No: 108 ACACA promoter 2
CTCCCAAAGTGATGGGATTACAGGTGTGAGCCACCGCTCCCAGCCCACA
G
TGATCCTTTTACAGCAAAGCTACTCCTTTGCTCTCAACCCTTCGTCATAT
TCTGTCACTTCCCAATAAAATCCAAACCTTACCAGATGATACATGATCTG
CCTTGATTTCCATCTAATTTCTCTCATTTGCTGTGCTCCAGCCACACTGG
TCTTGCCCTTTCTCCATGCCAAACAAATCCCCATTTCAGGGTCACTGCAC
TTGCTGCTTCTCTTCTAGGATAGTTTCCCTCTAGAAATCCATGTTGCTCC
TTCTCTCATTAAATGATCAGATATTTATGTGCCTCCTTAGCTAAGAGGCC
TTCCTTATCTACCCTAACTAAGAGAATCCCCATATCACTCTTATCCTTTT
ATCCTGATTTATTTTTCCATAAAACTTGCCACATTACTGGGCATTATCTA
TCATTTAGTAATGTGTATATTGCTTATTGACAGTCTTCCCTACTATAATA
TGAGCTCCATGAGGCAGAGACTTTGTTTTGTTCACTGCTGAATTCCCAGC
ACCTACAACAAGCCTACTCACTAAACATTTGTTGAATTAATCTTCAGGAT
ACATCAAATGTAAAATATCCTATCATATATTTATTGATGTGATGCATAGG
AAAATGTCTGGAAGGGTACATACCAAAATGTTAATTATTTATTTAACCTG
GTGGGAGGTGGTTTTTCTTTCCTTTTCTTTTCTTTTTGAGACAGAGTTTC
GCTCTCGTTGCCCAGGCTGGAGTGGAATGGCAAGATCTTGGCTCATTGTA
ACCTCTGCCCCCAGGGTTCAAGCGATTCTACTGCCTCAGCCTCCGGAGTA
GCTGGGATTACAGGTGCCTGCCACCACACCCGCGTAATTTTTTGTATTTT
TAGTAGAGACGGGATTTCACCATGTTGGTCAGGCTGGTCTAGAACTCCAG
ACCCTGTGATCCGCCCGCCTCGGCCTGCCAAAGTGCTGGAATTACAGGCG
TGAGCCACCGCGCCCGGCAGGGCTTTTTTTTTTTTTTTTTTTTGGAATGA
ACTTGTATTATGCATATACCAAAAAAGAATGAAAGGGTTCACACAGGCAT
AAAGTGTAAGTGAATTCTAAAAAAAGGTTGGGCAAATTTATCAAAGGCAG
ACTTCTAAATAGATGTAACAGTTATATAGTTCAGATAAACCATGGTTTGA
ATTCCCTTCCCCAGTACTTCCTAGCAGTGTGATCCCAAGTTATTTATGTC
AGTGCCTAGATTTCTTCACCTGGAAAATAGGGATAACAACAGGTGCCTAC
CTCACAAGTTTTAAATGAGACCGTATGTGTAACCTGCTTTGTGCAATGTC
TGGCATGCAATAAGTACTCGACAAATGGTAGCTATTAGAATCATCATTAA
CAGCAGTAATAAGAGATCTTGGGACATACCTAGCCCTCAAGATTGACATC
AGAGTAGACCACCACATCCTCTCATCATTGCGCCTCAATTTGGGCCTCTG
AAGCCCAAAGAGGGGATGGTTCTTTCCAAAGAAGACAATTTCGACGTTCC
AGGAGCATCTGATTGAAACGCACCCTCTTCACCCCTTAAAATCAGTCTGG
TTCATCCACGAGCAGCCCTTCGGGGCCCGGACTGGAGAGGCGCCACGGCT
CGCCGTCCCTGGGCCCAGTTCCCTCAGCCTCAATTTCCCTTGCTGCAACA
GGGGTGGAGATGGGAACGTTATCCCCAAACCCAGGCAGTCCCGGCTCGGC
CCGCCTCACCGCACTCCGGAGGGGACCAAACAGCCCCACGCGCCAGGAAG
CCTCAGGCAACGGGCCACGCGCCACACGGGCAAAGTGATTACTGGTCGGA
TCAAAAGTCAGGCAAGCGGCTCAGCCCCATCCTCCCAGTCCTCGGGCACG
GGGACAGCAGCAGGCGCGCGGCGGGGACCGGGAAAAGGCCAAGAGGGCGG
TGGCGGGCGCTCAGCACCCGGCGGGGGCCTCCACGGCCGAGGGGCGGAGG
CGGCGGGCGCGCGGCACGCGGCGCGCTCGCCACTGCGCCGCCCGGGGGCG
CGCACGCAAAGCCCGGAGGCGCGCGCGACCGGCGGCTCTTTGGCGCGGAT
TAGGGGGTCTCGGCGAGGTGAGGCGCCAGGCAGCGCTGGGCGGGCGGGCG
GGCGGGTGGCGGAGCGCGAGCCCCTCTAGCGGGACTAGGGAGAAGCGGGG
GCCACGAGGTGCGGCGGGGGAGGAGTCGCGGGCTGCGGGCCCCGGCGGCG
GGCTTGACGCGGCAGGCTGGCGGGCCGGCGGGCGGGCAGCGGCGGGCCTC
GCCGCTCTGTGGGGGAGGGCGGTGTGGGGCCGGGCCTGACCCCGGGGGCC
CGCCCCGCGTCTGGGCGGTGCCCCAGTGCCCCGGCCTCGCCATTTCCCGC
TCAGCCCTGGAGCGCGTAGCTCTACCAAGAATGGCCACTGTGCCAGATGC
CCCTGACCAGCGTTGCCCATTTGAATTTCCTAGCAGGCCCCCCAAAGTAG
GTATTTCAGTACCCTGTTAGAGCTGAGGCGCAGGTAAAATGACTGGCCCA
GGCCGGTCCCACCCCGTAAGGATTTGAACGTTGGCTCCACAACTCGGGAG
CCTGCGCCTTTCCTCCTCCCAACGTGGACTCCTGCCCGGCGAAGTGCCTC
ACTTCCTTCTCCCGGTAACTGATTCGAATTTGGCGATTGTTAGTTTGGAG
CTAGCGCCAGTGTTTCCACGGAAGTTGGTGTCAGAATGTGGTCTCTTGTG
AAAGGGGGAGAAAGAAGATTCTGCAGACTGCAATATTGTGCTTTTTTGCC
TTTTTTTTTTTTTCTCCTAGGCTGTACTGCAGTGCAGTGGCACGATCGCG
GCTCACTGCAATCTCCGCCTCCCAGGCTCAAGCGATTCTCGTGCCTGAGG
CGTGGGCCACCACGCCCAGCTAATATTTGTATTTTCAGCAGAGGTGGGGT
TTCACCATGTTGGCCAGGCTGGTCTCGAACTCCTGACCTCAAGTGATCCT
TCCGCCTCGGCCTCCCAAAGTGCTGGGATTACAGGCGTGAGCCACCACGC
CCGGCCCTACTATTTTTTTATTTTTTGAGATGGCGTTTCTTTCGCCCAGG
CTGGAGTGCAATGGCGCCATCTCAACACACTGCAACTTCTGCCTCCCAGG
TTCAAGCGATTCTCCTGCCTCAGCCTCCCGAGTACCTGGGATTACAGGCG
CCCGCCACCACACCTGGCTATTTTTTGTATTTTTAGTAGAGATGGGGTTT
CGCTGTGTTGGCCAGGTTGATCTCGAACTCCTGACCTCAGGTTATCTGCC
CACCTGGGACTCCCAAAGTGCTGAGATTACAGGCGTGAGCCACCGCACCC
GGCCTCCTACTGTGCTTTTTCGAGTGTTGATCAGTCCCTATTGACTGTGC
ATCCTTTACTGGACGTCTGTTTTTGTTTTTGTTTTTTGAGAGGGAGTTTC
ACACTTGTTGCTCAGGCCAGAGTGCAACAGCGCGTTCTCAGCCCACCGCA
ACCTCCACCTCCCGGGTTCAAGCAGTTCTCTTGCCTCAGCCTCCCAAGTA
GCTGGATTACAGGCATACGCCACCACGCCCAGCTAATTTTGTATTTTTAG
TAGAGACAGGGTTTCTACATGTTGGTCAGGCTGATCTCGAACTGCCGACT
TCAGGTGATCTGCCTGCCTCGGCCTGCTAAAGTGCTGGGATTACAGGCGT
GAGCCACTGCGTCCAGCCTTGACATCTGTATTTGAAATGACACAGCTATC
CCTTGTAAGACACTGCGCAAGTAACATACTCTTTCAGAGCTTGCTTGCTT
TTGAAAAAGCAGCTTGCCCCAAAAGTGTGGGAGTGAGGATTCAAAGAATC
CCCACACCTTGGTACCCACGGGTACCTAGCAAATTGAAAAAGTTCAGTAA
ATTTGGCAGTTATTTGATTGCTGCGTTCGTAATACACCCTTGCTAGACTC
ATTTTTCTTCAGAGCTCAACTTTTCCTGAGAGGACAGCAAAGGGAGACCT
A
Seq. ID. No: 109 ACACB promoter 1
TGTAAAGCTCTTAGCACAGTGGCTGTCATAGGCCACACACATGCCAGTG
A
CACAGAAAAACACTTGTCGCGACTTATTAGGAATCACTTCCTCTTCCCTG
CAACACTCCCTAACATTTCCTTTCTTCCCTTCTTCAACCCCTATGTGGGC
ATTAAAAAAATAATCCTCTGTTGAAATAACTGAAAAAATGGTCTCCCTGG
GCCCCCAAACACACCCAGAGTCCTTAGGAGCATCCTCACTGGATCAGGGA
ACAGATAGGATGTTAATTCAGCCTGGTCCATGCTGCTGAAAGCCCAGAGA
CTTTCCCCTTTGGAAAAATGCCTGCCCCTGCAGGGCACCATCCCCTCTGC
CCCCCAAAAGCAGTTCTAGGCTGCAGGCCTGGGAAGCTGTCCTTGATGGG
GGGATCTGCCTGTCCTGCCTGGGCTGGCCGATCCTTAGAGAGTGGCTCTG
ACACCTGACCAGGACTGACAGGTGCTGTGAACCAAATGTGAATCCTTTCT
GTCCATGAGTGGAGGAAGATGCTCACAGCAGAAGCCGCCATCACTATCAC
CTGCCTGGTTCTTTCTGTTTGGTGCAGCAGGAAGGGGAAGGCTCTATTTG
GTGTTGAGTGACTACATGAATCTTGGTTGATTCTCACAATAAGGCAGGTG
AGGTAGGCGAGGCTGGGTATTTTGCCCCCTTTTCTAGATGGGGACACTGA
GGTTTGGAGAGGGGAGGTGATTTGTTCAAGATCACACTGTCCATTGGTGG
CAGAGTCCTGGAGCGGTGGCTCACACTGCCCATGGTGCTCTGAGTGGAGA
TGGTGAATGCTGATGGCTCAGATGTGTACAGCCCAACTGCATCCAGCACT
GCACTCAGTGCCCCAGGGGGGATGAACTCATTGAACTCTTAACAGCCCAG
CAATGATGTAGGTACCATCCTTATCCCCATTTTGCAGATGAGGAGACTGA
TGATCAGGGAGGGCATTTAACTTGCCCAAGGCTTCCCCAGCTTGTAAGTG
GCAGAACCGACATGTGAACCCAGGTCAGTTTCCAGGGGACCAAACAAGGT
GCCCGGTGACAGTTGACTTTGGCCGCCAATCATGCCTCAATATAGCGGAA
GAACTGGCCTGGAGCCGGGGATCTTGGTTGGTCCCAGTCTTTGGCTCTGT
GGCTAATTGCACATGTGACCTCCTGGCCACCATTTGCTCATCTACAACAC
AAGCACACTGGACTCGATGATCCCTGTGCTTCCCCGCATTCAGCTGTAGG
TTGCAGCAGGTGGCTTTTGAGAAACACTCTGATCTGTGACACCCCAGATC
TACAGGCCGAGTGCAGGTGAAGGTGTCCACCAGTCAGCCTCTTACGCCCC
AGCAGCCACACTTGGCCTGTCCCCATCAGTTTAATTATCTCTGAATTTGC
AAAAGGACATTGCGCCACTTGCTGAAGGGCCCCTGACCACCCCCTCTTTG
TCCCGATACTGTTGCTTCTCTTTCCTGCCTGGGCTGACCTGTGTGTCCAG
CTGGCCATTCGTGCCCAGTATAGCCAAACACAAGTTGTGCTGAGTCTCAC
CCTCCCTTCTGGACAGGCGTCTGACAGATGAACCCCTTGGAACATCTGTC
GATGCTGTTTTCCTGTCTACCCAGGGCTGTTGTGATTACAGGACTGTTTA
AAGCCTGTACCAGAGATGGAGAGTTGGGCAAGAGGGCAATTGCATGAGCA
GTGAAGTAGGGCTAAGTACAGAAATGTGGCAAGCTGGAGGGAGGTGGTCC
TCAAGCCCACGAAGCCCCTGGGAGCACAGATAGCCTCTTAACTACCTAAG
CTTGAGGTCAGGAGAAGGCTGAGTGAGGACCTTTGAAATGGCTTAAATTT
ATGGGTGACAGATAAAGGCATGCTGCACTGCCAACTTGGGGGAACGCTCA
CCCATGTCGGAGTGCTGGCCGGTCCAAGCCCCACCCCCATGGCCTGATGC
ACGCCACCAGCCTCCCGCTGAAAGGTGACACTCTGCCAGCTGGGTTCCCT
TAGTCACCCTGTGGGCGCCTGTCAGCCTCACTCAAGAATGGAGCTGGTGG
CTGCATCCCCAGTGACGGTAAGTGGCAACAAGTGCTGGGGAGTTAATCTT
GGTGGGAGGAAAAATCCAAGCATGGATGGGGATTGCTGGATGCTGACATT
CTGTCCCCTCTGTCCTTTAAAAACCTCACCAGGCATGGGGATGAGTGTGC
TGTGGGTTGTGGAGGGACTGGAAAACAGACTCCTGACTCCCGTCCTTGCC
CTGCCCTTGCCTGGTGACTGGCCAGCACTTACCAGGACTGAGCAGTTTCT
CGTGTTCATTGTGGTGAAGGCCACGATCACTCTTTCAACAGGCAGCTGTT
GCTTGGGTTAGGTTTGCAAGACATAAAAATAGCTTTGTCTGGCATCTGGT
TTTCGGGTCCGCTGGGTTATTTCAGCGAAGGGCAGCAGCTTGTCATGATA
GCGTCCACAGAGGTTCACTGGAAGGGCTCGTCTGGCCTTCCCTGCTCATG
GAATCAAACTGCGTGATCGGTGCTCATTCTTGACCACGGCCCCCCCACGC
CACCGTCTGGCACCGTTAGTTTGCATGTCGATCTGCGGCTGTGTCTTAAA
GGTACACAGCCAGGTGTCTGTCTGTTTTTAGGATCATATCTACGCACTCC
TTGTACCACCTGCTGCCTTATGGAATTTACTTATCTCCACTGCAGTGGTG
GGACAGTGGAAAAGGTATGAGCAAGGGAAAGTTACTGATCCACAGTTTAC
CAAGCTGTAAAATGGAAGTCAAATAGTGACAATGACAATAATAACATCCA
TTGGGTTGTTCCTTTTTTTTTTTGAGATGGAGTCTCACTCTGTCACCCAG
GCTGGAGTGCAGTGGTATGATCTCAGCTCACTGCAACCTCTGCCTCCTGG
GTTTAGGTGATTCTCCTGCCTCAGCCTCCCAAGTAGCTGGGATTACAGGC
GTGCACCACCACGCCCAGCTAATTTTTGTATTTTTTAGTAGAATGAGGTT
TCACCTTGTTGGCCAGGCTGGTCTCAAACTTCTGACTTCAGGTGATCCAC
CTGCCTCAGCCTCCCAAAGTGCTGGGATTACAGGTGTGAGCCATCACGCC
CGGTCCCATGGAGGTGTTTTAAGGATTGGAGGGAGGTGGTGGTGAGGGTC
TTGGGAGCTTGAAGACACTCCACAAATTTGATGGCTTGTATGTGTGGTTG
CTATATAGGTACAGATGTTATGCAGAGAGCAGCAGTGAGGTGAATGGGGT
GAGAATTCGTATCCCCATTTTATAGATGAGGAAACTGAGGCTCAGAGAGT
TTCTTCTGTAGCTTTCTCAAACAGTCCTCCGATTTGTCTCTGTGTATTCA
CTTGGGAAAAGTTCTTGTGTGGGGTGACCTTTGGGTCCCACGTCATTATT
CCGCAAAGAAAAGGGAAAGGCTGTGAGGTTTTTCATGTAGGTAACCCTGG
CAAGGAAGGTTTGGGGGATTAGATCCAACAGAATAACAGATTGTAAAGGA
TTTGCAGAGTGAGAGAAGGGCTGAACTGGGGAGAAGAGCTGGCCACTGCA
TTCGTCAGAGCCTCCGCTGTACAACTTGCAGCTTTACCTGCCATCCCTCT
ATCACCTGCCGTGCGTGTGTCTAACCATCTTGCCCATTGACTCATCCACT
CACCTTCTCTCTCCACACACCACCTACTCATCCTTCTATCCGTAGTTAAT
CAGCCATCCATTTGTCCAGCCAGCCAGGATTTTCTAAGCAAGTTCCTGAA
GAACTTATATCATGTAAAACTTGCATAATTTGATTGGCTTTCCAACAGGA
ATCAATGTAAACAATGTATTCTCACACCTCATAATTCACAACCATTGTAG
CACACTTTTGTCTTTATGCTGCACTTCTCTTTCTCTCTCTCTCTCTCTCT
CTCTCTCGTTGCCCAGGCTGGAGTGCAGTGGCGTGATCTTGGCTCACTGC
AACCTCCCCCTCCTGAGCTCAAGCAATTCTCCTGCCTCAGCCTCCTGAGT
A
Seq. ID. No: 110 ACACB promoter 2
TGTAATCCCAGTGCTTTAGGAGGCAAGGCAAGAAGATCGCTTTAGGCCA
G
GAGTTCAAGACCAACTTGGGCAACATAATGAGACCTTGTCTTTAAAAAAA
AAAAATTTTTTTTTTTTTAATTAGCCAGGGGTGGTGTGTGCCTGTAGTTC
TAGCTACTTGGGAAGCTGAGGCAGGAGGATCATTTGAGCCCAGGAGTTCG
AGGCTGCAGTGAGCTATGATCATGCCACTGCACTCCATCCAGCCTGGGCA
ACAGAGCAAGATCCTATTTCTAAAACAAAACAAAACAAAACAAAAAATGT
AACTTGATACCTGTGACTTTCTGAAACTAACAGCTGTTTGAAGAAATATA
TTTCTATTACCGTTCTTGTGTTTGATGGAAAGAGTTTATGAATAACTTTG
GTTTCAAGCCTTCCAATTATATTCTGGAAAATACCTGTGTCTTAAAGAGA
TAGTGTCTAGGGAGAACTGAATCACTGCAGAGTTCTTCACCTGTATGTGT
GGGTATGTCTAGTCTTTTTGCAAGTAATGAATTTCATTAGAAAGATATTA
AGTGCTTGTCATGAGCAAGCCCCTGTGCCAGAAACTCAATATAAATCATT
TCATTTTCTCACCCATATGGTGAAGTAGTTGATGAGGACACTGAAGCTCA
GCCATTTGCTCAAAGTCATGGCTAATGGAGGGAGGATTCAAACCCACATC
AGCTTGATTTCAAAGCCTAATAGTCCTGCCACTGTCAACACCCTGCCTAG
ATCATGATTATTTGGAATCACTGGGAAAGAATTTTTTTTTCATGAAGTTA
GAAGCCTAAGAATTTTTTTCAATGGAATTTTATAAAAATGTCCTTGAATC
ACAGATGGCTCTCCCTGGAAACATAGGTTTGTTTGAGAGTCACCTCTTTT
ATTGTCTTAATAAATAGACTAAGAGCCATCACAAGGGCTACTGTGGCTAG
AGAACATATGACTCGGGCCGGGCATTGTGCAGGCATTTGACCTGCATTCC
TTCACCCCGGTTCCTGTAACATGTACCTGGTGCTGTGGGGTTACAGCTGA
TAATGCTGAGGTTTCCAGAGCACATGCTTCCAAGTCCAGTGGAGAAAATC
CCAAGAAGTCCAATATTCAATGACTGTTCCTTATTGCAAAATAAGCCCAG
AAACAAAGTAGCTGTGAACAGGCTGCACCAGCCCCAGCAGCAATTTCCAT
TTAAACCATTTTGCACAGATCCCGTGCTCAGCGCTCTCGCCAGTTTCTCC
ACTTGCTGTTTAGTAAGGCCATCATTGTTGGCAGGAAGTGGGTTCCACCT
TCCCTGTTGCCTGAGCACAATGTGCTCCTATGTGGTCTCCACTTTCCTGC
TGCCTAGTTGGCTGGGCTTCTCCAGCTGGGCCTTGGAGGCGACCTTTGTC
CCACATGCCAGCCCTCCCCACGTTGCAGAACACTATGTAACTGCAGCGTG
TGCTGAATTTCCTCTTCTGTGCTCAACCCTAGCAAGAGCCAGTCAACCCT
GAATGAGAATGTGGCAATGGAGCTGTTTTTTTCTTTTATCCCCAAGTCAC
AAGAAATTGGAAACATTCCATTAAGTGGCGTGATTTGAGAGGGTTTCGCC
AAATCTTTTCAGGGGAGAGAAGCCAGGTGACCTGATAGCGATCACCTTCC
CATTCACGGTTTTCAATGGGGGCTTGTTTCTCAAATGACCAACACCTGTA
GGCAGTTAATTTTCAGCATGTCACAGGAGGGCCCAGATGACTTTCTGCCT
ACTGCTTACGACAACATGGACTTTAGATGCCTCATGGCTTCAACAGCTAA
GAATTATGAAATAAAGCGAGTTATGGGATTAAAAGGCAGATAGCAGAACT
CTCCCAGGAGTAGCCAGGGCTGCACAAGTGGTTTGACGTGGGATCTTTCT
CATCTTTGCCCCGGGTTGTTGCTGCTTTGAAGGTTAAGTGCTCCAGGGAT
ATTTTTAAACAGCAGGGGCTGATTAATCTAAAAGTGACATTTGCAGTCCC
CAGAGTAAGCAGCTAGCAGGCTTAGATTCAGGCCCTCAGCAAACAAGGAA
CCTGGAAAATGTAACCCTGAATGCACGGTGGGGAGGACATGGCAAGAGAA
AAGCGGCAGGAATAAAGTGGTAAGTCAGCTCGCCCCTCCCCGGGGTCCGG
ACCATTCTGCCAACGTCCAGGTAAATTCGAACCCAATGTATAGTGAGAGC
AGACCCTGTCTGAAGGCATGCACTTGGGGTCCCCGGGGAAAAACGACTCC
CGTCATGTTTGAAGGAAGCTAATCAGAAGAGCTCGAAATGTAACGCAGAT
GAAAGAGAAGGGCCTTTCTTTGCTTCTATAAATGGTGGTGTGGATCCCTG
GACATGCTCCCATATAAGTTCCACAACGTTTTATAAACAATTCTGTTTTT
TGCCTCCAAAGCTACTGTCTTAAGGAATAGTATTCTGTTTCTGTTTCCTC
AAATGGTTGTTCTTTTTTTTTTTTTTTTTTTTTTTAAAGCAGACTAAGGC
TGGGCACGGTGGCTCATGCCACCCAGCACCTTGGGAGGCAGAGGCAGGTG
GATCACTTGAGGTCAGGAGTTCGAGACCAGCCTGGCCAACATGGCGAAAC
CCCATCTGTACTAAACACATAAAAATTAGCCAGATGTGGTGATGGGCACC
TGTAATCCCAGCTACTTGGGAGGCTGAGGCAGGAGAATCGCTTGAACCCA
GGAGGTGGGGGTTGCAGTGAGCTAAAATTGCGCCACTGCATTCCAGCCTG
ACTGACAGAGCAAGACTCTGTCTCAAAAAAAAAAAAAAAGAAAAGAAAAA
GAAAAAAAGGAGACTAAGAGTAAAGCGTTTGTAGTGGTCCTATATCCTAT
ATTTCTAGGAAGACATGACTTTCTTTCAATGTTGAAAAAATGCATTCCAA
AATAATATGCAAGTTAAAAAGTCACTCAGCACCGTGCAAAAGTCTCCTTG
CTGTTATTCCTGTCTAACCCTCCTCCTCCAGGTAGCCACTGTGCACATTT
TGGTATAAACCCCCCAGACCTTTTATTCTGTACACTCACACTGTACACAA
CTCTCTCCCTCATGTCTATTGTGATTACTTTTATTTTTTAATATCAATGG
GCATCCATATAGGTTGGGTGACCAACCATCCCACTTTGCCCAGGACCGGG
GAATTCCCGGGACATGGGACTTGCAATTTAAAAACTGAAAGGTGAAGATA
TTATTATTATCATTATTATTTGAGATGGAGTGCTCACTGCAAGCTCCGCC
TCCTGGGTTCACGCCATTCTCCTGCCTCAGCCTCCCAAGTAGCTGGGACT
ACAGGCACCCACTACCACGCCCGGCTAATTTTTTTTGTATTTTTAGTAGA
GACAGGGTTTCACTGTGTTAGCCAGGATGGTCTCGATCTCCTGACCTCAA
GTGATCCGCCCGCCTCGGCCTCCCAGAGTGCTGGGATTACAGGCATGAGC
CACCGCGCCTGGCCAAAGATATCATTTTATAAATTGCATGTTTCTGCTAA
TAGTGACATGGAGTTCTTTACAAGATTTCTTTTTATGACTTCTCCAAGTT
TTAAGTGGAAAGACCCAGCCAATCCTAATTCATGTGATTTAGAGATGAAA
ATATCTCTAATCTGTAACTTTCTTTCAGAAGCTCTTAAGAGTTTTATTTG
CTCCTGCTCAGGTGTTGTGTAAACTCGCTCATACTTTCAACACCTTTAGG
TCTGAGACAGAAATTTGAGTATTACATTACATGAGATACCCAGTCTTCAT
GGGGAAATAAACATTTTAATCTTTTGAAATGGAGGTTTCCTTGAAGTCTT
ATCCCAGTAGAAACTTTGACCTTGTTCTTTTTTTTTTTTTTTTTTTGAGA
CAGAGTCTCGTCCTGTCACCCAGGCTGGAGTGCGATGGCACAATCTCAGC
TCACTGCAACCCCTGCCTCCCAGGTTCAAGCAATTCCTCAGTCTCAGTCT
CCCGAGTAGCTGGGATTGCAGGCAAGCACTACCACGCCCAGCTAATTTTT
G
Seq. ID. No: 111 ACACB promoter 3
TCTTCGGGGAACATGGCGTGCCCGTCGTGGCTACGTTCTGCCAAGCCCT
G
TGACGTTGGAGGGGAGCCGCCTGCATCCCCCGCTCAGCCAGTGTTTCTAG
ACTCCGAGACATCTGGAACTCGGAAGTGAGGCCAGGGCTCCAGGAAGACT
CCCTGATGACGCACTGGCCCGCAGCCCAGGCTCAGGTAGTGGGGGCTGTC
AGGATGATCTGTGGGATCCCCCAGTGTCCGAAGAAAGAAGCCACAATTGT
GTTTTTTTTTCTTTCTTTCTTTTTCTTTTTTTTTTTTTGAGCGAGTCTCA
CTCTGTCGCTCAGGCTGAAGTACAGTGGCGCGATCTCGGCTCACTGCAAC
CTCTGCCTCCAGGGGTCAAGCAATCCTCCCACCTCAGCCTCCCAAGTAGC
TGGGATTACGAGCATGCACTACCACGCCCGGCTAATTTTTGTACTTTTAG
TAGAGAAACTTTTAACTAAATGGGCAAGAAAGAATGCATCCAGCCAGAAC
CTCGAGACTAGCAAATGCATTTCTTAAAAGGGCAGGGAGGGAGACCAGAG
TTTGCTTGAAAATACTGCTTCCTTTCCTTATCTATGTAGGGAAGGGTTTA
TCATTCTACATTTTGTGGTCAGCAAGCTGGCTTTCACGGGAGGAGGATGC
ATGGTCGATTTGCCCTCTGGTAACTTTGGGATCTTCCTCTAAACAACAGT
GGACTCCCTTAGCCAAGGAAGAAGTGAAGGGATAATTGTGCTTACACCAG
TTAGCTTTTCACACTTCTCTCTCTCCCGGTGCAGCACTGCCGGTAGGGGG
AGCAGTGAGCTCCAAGTTTCCAGGAATAATTCAAAACACAATTATTTTAT
ATTTTAAAATATAAAATGTATATTTTAAATATATATACATACATTTCTAT
ATAATATATACAAAAGTTCAATATACGTATATTGAATATATATGTATATA
AGTTCAATATATATGTATGTATATTCAATATATATGTATGTATATATGTA
TATATATTCAATATATATGTGTATATATTGAACTTTAGTCATGTGGTGAC
ACTTTAAACAGTACACAGAGCAGCTTCTGGGTCCATGACCCTCCAAAAAT
TTAATTACTGATTCTTTAGAAACTAGGCAACTCAGCCAGGTGTGGGGGCT
CACGCCTGTAATCCCAGCACTTTGGGTGGCCGAGGTGGGTGGATCACCTG
AGGTCGGGAGTTCAAGACCAGCCTGACCAGCATGGAGAAACCCTGTCTCC
ACTAAAAATACAAAATTAGCCAGGCGTGGTGGCACATGCCTGTAATCCCA
GCTACTTGGGAGGCTGAGGCAGGAGAATCACTTGAACCCGGGCGATCGCA
CCATTGCACTCCAGACTGGGCAACAAGAGCATAAGAGCAAAACTCCGTCT
CAAAAAAAAAAAAAAAAAAAAACCAACCAGGCAACTCATTTTGATGAACT
CTTAGCTATGTGGTAGTTGCTTTTTTCATGTGTCAAGTCTGCTATATTCC
TGCACTCAGTATGCAAAGATGCATAAAAGGCAGTCTTAGCCTTTTAGGTC
AGCGGTTGGCAAACTTTCTGTGAAGGGTCAGGTGGTAAATATTTGAGGGT
TTGCAGTTACCCGTTCTCCGTGGGAACTGCTCAACAACTCACTGCAGAGA
AAGCAGCCGTGGGCAAAACGTACAGGAACGGGCGAGGCTGTGTTCCAATA
AAACTTTATTATTTATGGACACTGAAATTAGATTTTAATATGATTTTCAT
GTGTCACGAAATCTTTTTTTTTCCTAAGCATTTAACAAATGTAAAAACCA
CTCCTAGCTTGGGAGCTATTGGAAAACAGGCATCTGGAACCACAGTCTGC
AGACCCCTGTGTGTACTCACTGTCAGTGCGGTGGGAACAAAAAGGCAGCA
ACGGCTGGGTCAGCCCAGGAAAGAAGACATTTCCCAGTGGAGAGAATGTC
TGAGTGGATATTGAACTTTCCAGGCCACAAAGAGGAACAAGAGCATTCAA
CACTGAGGAGCCTGCCTCTGCAAAGGCAGGACCCACATGCTGGATGTGGA
GCTGGATGGTTCCACGTGGCAAGGTTTGGGAGGCTTCTGAGAACCTGGCC
TAATGATTCCTGTTTCCTCCCTTTTCAGGTGAGTTAGGGAGATCTCTGAC
AGCTTTTGGAATTGGTAACACAAGGAAGTCAGTTATTAGCAAATGTTGTA
CTGGAAACGTCCAGTTATAAAATAATTAATTGTGTGAAAGTCACTGCCTG
CTTTTGATCTACATCCAGCAGGGGAAGGTCAGGGACTATAAATTGTTTGC
ATTTTTTAAAGATAGACTTTAGCATGTTGCTAGTATGCTTGCATTATTAG
ATAATACATCTCTGTTCAAATTCACTTTCTGCTTCAGTTTGCTTCTCATA
TAAAAGTCTTCGCAAGTATGTTTGGTGGAGAAGCAAACTTTTTGGCGTAT
GCGTGTGGTGTTGGCTTTTCATCCTTGCCAAACCTTCAAGATTTGCAAGG
AAAAACAGGTTGCAGGGAGGTGCTCTGGGTTACTTTGAAAAGACCTCACA
TGCTGACTTGTGCTGACTCAAGCTGAGGAAAGCTAGAACATTCCTCTGCT
GGAGATGGCCTCCTTGTTTAGAGTCCTTCTGACACTGTAATTTGGGTCTG
ACAACAGGAAGTTCCTTTGTAGTTTAAGTCAATGGGAACCAATTTTTTTT
TCTTTTTTTTGCGATAGAGTCTCACTCTGTTTCTTAAGATGGAGTACAGT
GACTCAGTCATGGCTCACTGCAGCCTCAACTTCCTGGGTTCAGGTGATCC
TCCTGCCTCAGCCTCCTGAGCAGCTGGGACCACTGGTATGCACCACCATA
CCTGGCTAAGTTTTTAATAGAAATGGGGTCTTGCTAAGTTGCCCAGGCTA
GGGAACCAGTTTTTAAGTTTTTGAAATATGCTTGGAAATATAAATATTGG
TGAGCGGAGCAGACCAGGAGATCAATAATTTATGTTCTCTGCTTGTTAGT
TGTGTATATAGCATGTTCTTTTGTCTCTCTTAATGATGTTTGGGGTGAGG
GTGTGGGAGAGGAAGGTAGGGAGCCTTCCAACAAGAAGCTAGTGCTCCTC
CAGCCTAGAGAAGCTAAAACATGCAAAGCCAGACCCCATCCCTGGAAATT
CTGATTTAATTGGTGGGGGAAGGCATGTGAATTCTCACAGAGGGTCCGGT
TTGCAGCAGGACTGGTGATTTCTTTCCAGGTGCCTTGTTTTGTTAGACCA
TGTGGTCACTTAATAATTTTTTTTGGGCCAGGTACAGTGGCTCATACCTG
TAATCCCAGTGCTTTAGGAGGCAAGGCAAGAAGATCGCTTTAGGCCAGGA
GTTCAAGACCAACTTGGGCAACATAATGAGACCTTGTCTTTAAAAAAAAA
AAATTTTTTTTTTTTTAATTAGCCAGGGGTGGTGTGTGCCTGTAGTTCTA
GCTACTTGGGAAGCTGAGGCAGGAGGATCATTTGAGCCCAGGAGTTCGAG
GCTGCAGTGAGCTATGATCATGCCACTGCACTCCATCCAGCCTGGGCAAC
AGAGCAAGATCCTATTTCTAAAACAAAACAAAACAAAACAAAAAATGTAA
CTTGATACCTGTGACTTTCTGAAACTAACAGCTGTTTGAAGAAATATATT
TCTATTACCGTTCTTGTGTTTGATGGAAAGAGTTTATGAATAACTTTGGT
TTCAAGCCTTCCAATTATATTCTGGAAAATACCTGTGTCTTAAAGAGATA
GTGTCTAGGGAGAACTGAATCACTGCAGAGTTCTTCACCTGTATGTGTGG
GTATGTCTAGTCTTTTTGCAAGTAATGAATTTCATTAGAAAGATATTAAG
TGCTTGTCATGAGCAAGCCCCTGTGCCAGAAACTCAATATAAATCATTTC
ATTTTCTCACCCATATGGTGAAGTAGTTGATGAGGACACTGAAGCTCAGC
CATTTGCTCAAAGTCATGGCTAATGGAGGGAGGATTCAAACCCACATCAG
C
Seq. ID. No: 112 ACACB promoter 4
CTGAATGCTCCACTTTGGAAGGGGGAGAATTTAGAGGGCAAAGGGGAA
TC
GCACAGGGTCTTAAAGTGCAACAGCCACAGTCCTTCCTTTTTGGGGAAAA
AAAAAAAAAGTCCCGGCCGGGCATGGTGGTTCACGCCTGTAATCCCAGCA
CTTTGGGGAGGCCAAGGCGAGCGGATCACGAGGTCAAGAGATCGAGACCA
TCCTGGCCAATATGATGAAACCCCATCTCTACTAAAAATACAAAAATTAG
CTGGGTGTGGTGGCACGCGCCTGTAGTCCCAGCTACTTGGGAAGCTGAGG
CAGGAGAATCGCTTGAACCCGGGAGGCGGAGGTTGCAGTGAGTCGAGATC
ACGCCACTGCAGCAAGACTCTGTCTCAAAAAAAAAAAAAAAAAATTTAAA
AAGTCCCAAATCTGCCACCATTTATTCTTGATCTTTTTCAGAAGCGGCAC
CATGTACTACAGACGGCTCATCCCTCCCCTTTGTCAGTGTATAGAGGGTT
CTTTGGATGTAGACACTTTTCAAAGACCAATGAGCTGCTGCAGAAGTCTG
GCAAGAAGCCCATTGACTGGAAGGAGCTGTGATCATCAGCTGAGGGGTGG
CCTTTGAGAAGCTGCTGTTAACGTATTTGCCAGTTACGAAGTTCCACTGA
AAATTTTCCTATTAATTCTTAAGTACTCTGCATAAGGGGGAAAAGCTTCC
AGAAAGCAGCCATGAACCAGGCTGTCCAGGAATGGCAGCTGTATCCAACC
ACAAACAACAAAGGCTACCCTTTGACCAAATGTCTTTCTCTGCAACATGG
CTTCGGCCTAAAATATGCAGAAGACAGATGAGGTCAAATACTCAGTTGGC
TCTCTTTATCTCCCTTGCCTTTATGGTGAAACAGGGGAGATGTGCACCTT
TCAGGCACAGCCCTAGTTTGGCGCCTGCTGCTCCTTGGTTTTGCCTGGTT
AGACTTTCAGTGACAGATGTTGGGGTGTTTTTGCTTAGAAAGGTCCCCTT
GTCTCAGCCTTGCAGGGCAGGCATGCCAGTCTCTGCCAGTTCCACTGCCC
CCTTGATCTTTGAAGGAGTCCTCAGGCCCCTCGCAGCATAAGGATGTTTT
GCAACTTTCCAGAATCTGGCCCAGAAATTAGGGCTCAATTTCCTGATTGT
AGTAGAGGTTAAGATTGCTGTGAGCTTTATCAGATAAGAGACCGAGAGAA
GTAAGCTGGGTCTTGTTATTCCTTGGGTGTTGGTGGAATAAGCAGTGGAA
TTTGAACAAGGAAGAGGAGAAAAGGGAATTTTGTCTTTATGGGGTGGGGT
GATTTTCTCCTAGGGTTATGTCCAGTTGGGGTTTTTAAGGCAGCACAGAC
TGCCAAGTACTGTTTTTTTTAACCGACTGAAATCACTTTGGGATATTTTT
TCCTGCAACACTGGAAAGTTTTAGTTTTTTAAGAAGTACTCATGCAGATA
TATATATATATATTTTTCCCAGTCCTTTTTTTAAGAGACGGTCTTTATTG
GGTCTGCACCTCCATCCTTGATCTTGTTAGCAATGCTGTTTTTGCTGTTA
GTCGGGTTAGAGTTGGCTCTACGCGAGGTTTGTTAATAAAAGTTTGTTAA
AAGTTTGTTTTGTGCAAGTGTCCTTTGTGCGTCCAGGCCAGGGCATCCAT
GGACGTCCTTGGGCTGCCCTTTCCCTTGGCGCCTCCCAGGGTTCCCATAG
CAACCACCGTCTGCAGGAGGGGCCGCCCTTGCCCCTCCTCCCCGCCCTGC
CGCTCAGTGGAACGGCCCAACCCTCCCCTGGCTGCGGTGAGCGCTGGGCC
CAACCCCCGGCCTGGAGCAGCGCCCCAACTCCGAGCACCGTGGAGCACCG
GCTGCCAGCTGAGACCCCAGAGGGGTAACTAACGGCCTGAGGAAGGCATT
TCTTCGGGGAACATGGCGTGCCCGTCGTGGCTACGTTCTGCCAAGCCCTG
TGACGTTGGAGGGGAGCCGCCTGCATCCCCCGCTCAGCCAGTGTTTCTAG
ACTCCGAGACATCTGGAACTCGGAAGTGAGGCCAGGGCTCCAGGAAGACT
CCCTGATGACGCACTGGCCCGCAGCCCAGGCTCAGGTAGTGGGGGCTGTC
AGGATGATCTGTGGGATCCCCCAGTGTCCGAAGAAAGAAGCCACAATTGT
GTTTTTTTTTCTTTCTTTCTTTTTCTTTTTTTTTTTTTGAGCGAGTCTCA
CTCTGTCGCTCAGGCTGAAGTACAGTGGCGCGATCTCGGCTCACTGCAAC
CTCTGCCTCCAGGGGTCAAGCAATCCTCCCACCTCAGCCTCCCAAGTAGC
TGGGATTACGAGCATGCACTACCACGCCCGGCTAATTTTTGTACTTTTAG
TAGAGAAACTTTTAACTAAATGGGCAAGAAAGAATGCATCCAGCCAGAAC
CTCGAGACTAGCAAATGCATTTCTTAAAAGGGCAGGGAGGGAGACCAGAG
TTTGCTTGAAAATACTGCTTCCTTTCCTTATCTATGTAGGGAAGGGTTTA
TCATTCTACATTTTGTGGTCAGCAAGCTGGCTTTCACGGGAGGAGGATGC
ATGGTCGATTTGCCCTCTGGTAACTTTGGGATCTTCCTCTAAACAACAGT
GGACTCCCTTAGCCAAGGAAGAAGTGAAGGGATAATTGTGCTTACACCAG
TTAGCTTTTCACACTTCTCTCTCTCCCGGTGCAGCACTGCCGGTAGGGGG
AGCAGTGAGCTCCAAGTTTCCAGGAATAATTCAAAACACAATTATTTTAT
ATTTTAAAATATAAAATGTATATTTTAAATATATATACATACATTTCTAT
ATAATATATACAAAAGTTCAATATACGTATATTGAATATATATGTATATA
AGTTCAATATATATGTATGTATATTCAATATATATGTATGTATATATGTA
TATATATTCAATATATATGTGTATATATTGAACTTTAGTCATGTGGTGAC
ACTTTAAACAGTACACAGAGCAGCTTCTGGGTCCATGACCCTCCAAAAAT
TTAATTACTGATTCTTTAGAAACTAGGCAACTCAGCCAGGTGTGGGGGCT
CACGCCTGTAATCCCAGCACTTTGGGTGGCCGAGGTGGGTGGATCACCTG
AGGTCGGGAGTTCAAGACCAGCCTGACCAGCATGGAGAAACCCTGTCTCC
ACTAAAAATACAAAATTAGCCAGGCGTGGTGGCACATGCCTGTAATCCCA
GCTACTTGGGAGGCTGAGGCAGGAGAATCACTTGAACCCGGGCGATCGCA
CCATTGCACTCCAGACTGGGCAACAAGAGCATAAGAGCAAAACTCCGTCT
CAAAAAAAAAAAAAAAAAAAAACCAACCAGGCAACTCATTTTGATGAACT
CTTAGCTATGTGGTAGTTGCTTTTTTCATGTGTCAAGTCTGCTATATTCC
TGCACTCAGTATGCAAAGATGCATAAAAGGCAGTCTTAGCCTTTTAGGTC
AGCGGTTGGCAAACTTTCTGTGAAGGGTCAGGTGGTAAATATTTGAGGGT
TTGCAGTTACCCGTTCTCCGTGGGAACTGCTCAACAACTCACTGCAGAGA
AAGCAGCCGTGGGCAAAACGTACAGGAACGGGCGAGGCTGTGTTCCAATA
AAACTTTATTATTTATGGACACTGAAATTAGATTTTAATATGATTTTCAT
GTGTCACGAAATCTTTTTTTTTCCTAAGCATTTAACAAATGTAAAAACCA
CTCCTAGCTTGGGAGCTATTGGAAAACAGGCATCTGGAACCACAGTCTGC
AGACCCCTGTGTGTACTCACTGTCAGTGCGGTGGGAACAAAAAGGCAGCA
ACGGCTGGGTCAGCCCAGGAAAGAAGACATTTCCCAGTGGAGAGAATGTC
TGAGTGGATATTGAACTTTCCAGGCCACAAAGAGGAACAAGAGCATTCAA
CACTGAGGAGCCTGCCTCTGCAAAGGCAGGACCCACATGCTGGATGTGGA
GCTGGATGGTTCCACGTGGCAAGGTTTGGGAGGCTTCTGAGAACCTGGCC
TAATGATTCCTGTTTCCTCCCTTTTCAGGTGAGTTAGGGAGATCTCTGAC
AGCTTTTGGAATTGGTAACACAAGGAAGTCAGTTATTAGCAAATGTTGTA
CTGGAAACGTCCAGTTATAAAATAATTAATTGTGTGAAAGTCACTGCCTG
CTTTTGATCTACATCCAGCAGGGGAAGGTCAGGGACTATAAATTGTTTGC
ATTTTTTAAAGATAGACTTTAGCATGTTGCTAGTATGCTTGCATTATTAG
ATAATACATCTCTGTTCAAATTCACTTTCTGCTTCAGTTTGCTTCTCATA
TAAAAGTCTTCGCAAGTATGTTTGGTGGAGAAGCAAACTTTTTGGCGTAT
GCGTGTGGTGTTGGCTTTTCATCCTTGCCAAACCTTCAAGATTTGCAAGG
AAAAACAGGTTGCAGGGAGGTGCTCTGGGTTACTTTGAAAAGACCTCACA
TGCTGACTTGTGCTGACTCAAGCTGAGGAAAGCTAGAACATTCCTCTGCT
GGAGATGGCCTCCTTGTTTAGAGTCCTTCTGACACTGTAATTTGGGTCTG
ACAACAGGAAGTTCCTTTGTAGTTTAAGTCAATGGGAACCAATTTTTTTT
TCTTTTTTTTGCGATAGAGTCTCACTCTGTTTCTTAAGATGGAGTACAGT
GACTCAGTCATGGCTCACTGCAGCCTCAACTTCCTGGGTTCAGGTGATCC
TCCTGCCTCAGCCTCCTGAGCAGCTGGGACCACTGGTATGCACCACCATA
CCTGGCTAAGTTTTTAATAGAAATGGGGTCTTGCTAAGTTGCCCAGGCTA
GGGAACCAGTTTTTAAGTTTTTGAAATATGCTTGGAAATATAAATATTGG
TGAGCGGAGCAGACCAGGAGATCAATAATTTATGTTCTCTGCTTGTTAGT
TGTGTATATAGCATGTTCTTTTGTCTCTCTTAATGATGTTTGGGGTGAGG
GTGTGGGAGAGGAAGGTAGGGAGCCTTCCAACAAGAAGCTAGTGCTCCTC
CAGCCTAGAGAAGCTAAAACATGCAAAGCCAGACCCCATCCCTGGAAATT
CTGATTTAATTGGTGGGGGAAGGCATGTGAATTCTCACAGAGGGTCCGGT
TTGCAGCAGGACTGGTGATTTCTTTCCAGGTGCCTTGTTTTGTTAGACCA
TGTGGTCACTTAATAATTTTTTTTGGGCCAGGTACAGTGGCTCATACCTG
TAATCCCAGTGCTTTAGGAGGCAAGGCAAGAAGATCGCTTTAGGCCAGGA
GTTCAAGACCAACTTGGGCAACATAATGAGACCTTGTCTTTAAAAAAAAA
AAATTTTTTTTTTTTTAATTAGCCAGGGGTGGTGTGTGCCTGTAGTTCTA
GCTACTTGGGAAGCTGAGGCAGGAGGATCATTTGAGCCCAGGAGTTCGAG
GCTGCAGTGAGCTATGATCATGCCACTGCACTCCATCCAGCCTGGGCAAC
AGAGCAAGATCCTATTTCTAAAACAAAACAAAACAAAACAAAAAATGTAA
CTTGATACCTGTGACTTTCTGAAACTAACAGCTGTTTGAAGAAATATATT
TCTATTACCGTTCTTGTGTTTGATGGAAAGAGTTTATGAATAACTTTGGT
TTCAAGCCTTCCAATTATATTCTGGAAAATACCTGTGTCTTAAAGAGATA
GTGTCTAGGGAGAACTGAATCACTGCAGAGTTCTTCACCTGTATGTGTGG
GTATGTCTAGTCTTTTTGCAAGTAATGAATTTCATTAGAAAGATATTAAG
TGCTTGTCATGAGCAAGCCCCTGTGCCAGAAACTCAATATAAATCATTTC
ATTTTCTCACCCATATGGTGAAGTAGTTGATGAGGACACTGAAGCTCAGC
CATTTGCTCAAAGTCATGGCTAATGGAGGGAGGATTCAAACCCACATCAG
CTTGATTTCAAAGCCTAATAGTCCTGCCACTGTCAACACCCTGCCTAGAT
CATGATTATTTGGAATCACTGGGAAAGAATTTTTTTTTCATGAAGTTAGA
AGCCTAAGAATTTTTTTCAATGGAATTTTATAAAAATGTCCTTGAATCAC
AGATGGCTCTCCCTGGAAACATAGGTTTGTTTGAGAGTCACCTCTTTTAT
TGTCTTAATAAATAGACTAAGAGCCATCACAAGGGCTACTGTGGCTAGAG
AACATATGACTCGGGCCGGGCATTGTGCAGGCATTTGACCTGCATTCCTT
CACCCCGGTTCCTGTAACATGTACCTGGTGCTGTGGGGTTACAGCTGATA
ATGCTGAGGTTTCCAGAGCACATGCTTCCAAGTCCAGTGGAGAAAATCCC
AAGAAGTCCAATATTCAATGACTGTTCCTTATTGCAAAATAAGCCCAGAA
ACAAAGTAGCTGTGAACAGGCTGCACCAGCCCCAGCAGCAATTTCCATTT
AAACCATTTTGCACAGATCCCGTGCTCAGCGCTCTCGCCAGTTTCTCCAC
TTGCTGTTTAGTAAGGCCATCATTGTTGGCAGGAAGTGGGTTCCACCTTC
CCTGTTGCCTGAGCACAATGTGCTCCTATGTGGTCTCCACTTTCCTGCTG
CCTAGTTGGCTGGGCTTCTCCAGCTGGGCCTTGGAGGCGACCTTTGTCCC
ACATGCCAGCCCTCCCCACGTTGCAGAACACTATGTAACTGCAGCGTGTG
CTGAATTTCCTCTTCTGTGCTCAACCCTAGCAAGAGCCAGTCAACCCTGA
ATGAGAATGTGGCAATGGAGCTGTTTTTTTCTTTTATCCCCAAGTCACAA
GAAATTGGAAACATTCCATTAAGTGGCGTGATTTGAGAGGGTTTCGCCAA
ATCTTTTCAGGGGAGAGAAGCCAGGTGACCTGATAGCGATCACCTTCCCA
TTCACGGTTTTCAATGGGGGCTTGTTTCTCAAATGACCAACACCTGTAGG
CAGTTAATTTTCAGCATGTCACAGGAGGGCCCAGATGACTTTCTGCCTAC
TGCTTACGACAACATGGACTTTAGATGCCTCATGGCTTCAACAGCTAAGA
ATTATGAAATAAAGCGAGTTATGGGATTAAAAGGCAGATAGCAGAACTCT
CCCAGGAGTAGCCAGGGCTGCACAAGTGGTTTGACGTGGGATCTTTCTCA
TCTTTGCCCCGGGTTGTTGCTGCTTTGAAGGTTAAGTGCTCCAGGGATAT
TTTTAAACAGCAGGGGCTGATTAATCTAAAAGTGACATTTGCAGTCCCCA
GAGTAAGCAGCTAGCAGGCTTAGATTCAGGCCCTCAGCAAACAAGGAACC
TGGAAAATGTAACCCTGAATGCACGGTGGGGAGGACATGGCAAGAGAAAA
GCGGCAGGAATAAAGTGGTAAGTCAGCTCGCCCCTCCCCGGGGTCCGGAC
CATTCTGCCAACGTCCAGGTAAATTCGAACCCAATGTATAGTGAGAGCAG
ACCCTGTCTGAAGGCATGCACTTGGGGTCCCCGGGGAAAAACGACTCCCG
TCATGTTTGAAGGAAGCTAATCAGAAGAGCTCGAAATGTAACGCAGATGA
AAGAGAAGGGCCTTTCTTTGCTTCTATAAATGGTGGTGTGGATCCCTGGA
CATGCTCCCATATAAGTTCCACAACGTTTTATAAACAATTCTGTTTTTTG
CCTCCAAAGCTACTGTCTTAAGGAATAGTATTCTGTTTCTGTTTCCTCAA
ATGGTTGTTCTTTTTTTTTTTTTTTTTTTT
Seq. ID. No: 113 NRIP1 promoter 1
TCATTTGAATAAAATCAACAACGAACAGTCCTTTTTTTTTTCTGTTATGT
TCTGATGTATATTTTACTCACACTCCTAGGGCACTTTTAAAGGTAAGTTT
GGAAAGAGGTCTTTCTCCACCAGAGTATGAAACAGACTAGAACAACCAAT
CCTACAACCTGCAGGGCAGTAGCCGCTCCACTAATTGATTCTCCTTCTTT
GTCTCTAGGAACTATGCCCAGGCAGAAAAAAAGTTACTGTAGGTGATGAA
GCCAGTGCTCCCTGAACCAAATAAACCCTATCGACGTTACCGAACTGCCG
GGCAAAACCAGAGCAACTCACTTACTTGGAAGGTGAAAAACACTTCAACA
TACTCCAGGCGGCAACCGACACTTAGGGGCCAGGCAGATGAAACACCATT
TGTTTAAAAAGTCTATTATTTCACTGTCTCTTCAACAAAGGTGGAAAACT
GATGATTAAACACTGAATAATGCCACCCACTTACTAAACCTATGATTCAC
TAATAAGCAGGTTCAATGCAATTCATAACTTTAAAGAATGAATTACCGAA
GCCCCTTGCTTCACAAATTCCCCCAAGAAACAGAAAGAGCATTCATCACA
TATAACCCGACAACCATTTTTACTATGGACACCTACAGATGGCTTCTCTT
CCTCTAAGGACACAATTTCCTTCACAATCCCACATGTAATCATTTACAAT
ACCAGAAACAAACTCCAACTCAGTTCCACCAACTGGGTTTTTAAAAATCC
TTGTGCCTAAGAACGTCTTTTTTCCCCAAAGTAAAATGTGCCATTAAAAA
ATATTTTTAAAACGCAAAAGTGCTTATCATTCCGTACTTCTTTTACTACC
TTCAGCAGCCAATTATTGACGTGATAAAAGGTAGAAACTCCTGGAATGCT
TCAGAGATCAACATGTGTCACAGACTCCCAGCCTGTCCGCCTCTAAGACA
CAGTTGGTTCCTTTGTTCCTTCTGACTGAACACCAACTCCTGATCAAAAT
TGGGAAACTACTTTGGAAGGGATGGGAGGGTCTGCAGAATACATCCACGG
AAACCTTCATAAAACAATATTCAATCGTAATAAGGTGGACACAGAAGAAG
GGAGAAAAGGCAGCCTCCCGGACTTCCACCGGGCTCACCCTAAATGGGGG
CCGGCCCCTGGCTCCTGGTAAAATGATGGCTGAGGCCTCGGGTCCGTGAG
CCTCGCCATCGGGAGGGGACCGCGAGTGCGGGCAGAGCGGCCCGGGGTCG
GGTGGGGGCCGGGCGAGCTGTCCCTTTTCCTATTCACTTTCCCAGCCGGG
TATCCAGCGAGCAAGGAGCAGGGAGGCACCCAGGCGAAAGGCGCCGCCCA
GGATCCGGCTGGACAGCAGGACGGAGAGCGGCGAGGCGAGCGGTGCCCGG
GGTTCGGGACCGAGAGGGCAAGCGAGCGGGGGCAACCCCGACCCGGCCCC
CCGAGTCCGGCCCCGCTCAGGGGCCACGCGACGGCCGCCCCCTTGCCCAG
AACTGGCACGGACGGGCGGGCCGACCCGGGGCCACTCCACAACTTGCCGG
CCCGCCGCCCCGGCCCCCCGCGAGAACCCGGAGACTCGAACCAGGCAGCA
GAGGCAGGATTTCCCCGGAAACGCGCCCTCCCCGCCGCCCCGACGCCCCC
GCGGCCCTGCCAGCCAAGCGAGAGCCGACGCGGGCCGCGCTCCCGGGCCG
TTTCCGGCGGCGCCGGCGGCCGCTACTCCTGGCGCCCTGGCCGCGGCCGT
CACTCACCCCAGGCCGCCGCGGCTCCCAGCCTCCGGCTCCGTCAGGCTCG
GTCCGCGAAGGCGCCTGCCGCCCCGTCCTGGCCCGGCGCCCCGGCGAGCT
CTTCCCTCCGACCAGCGGCGCTCACGGCGCAGCGGCGGACGCGAGGCCAC
GGGCGGACGGGCGCGCGCGGGTGGCGGGCGGGCGTGGGGCCGGGTCGTCC
CTGCGCCTCGCCGCCGCCTCCTCCGCCGCCGCCTCCCGAGTTCGGGGCTC
TGCTGCAATGTCTGCGAGGCTGACTTTGAGCGCCTCGCTCACCGCCCGCC
CGCAGCCGCGCTCCTCCCTCCGCCGCCGAGTCCGCCGCGGCCCAGTCCCT
CCCCTCGCCGCCCCACGCCATTCAGCTCTTCAGCCAAGTTTCTTAACCCT
TTTCTCCCCCTCCCTCCCATTGTCACCCGGTCAGGAGGAAGCCGCGCGGA
TCCGGGCGGCTCACAGGAGCTGCGGAACCCCCTCTGTTTCGTGGATTTCC
TTCTCGCCTGCTCTCCGGGTGTCCCAGACGGGGCTGCAAGACCCGGAGGC
AGCGAGAGGACCGCGTCCCCGAGAGAGGGGCTGCACGGCGCGCCCGGGCC
GTCCCCCGGGCTCCCAGCTCTGTCCAGCCGCCCGCGACCGCTGCAGGCGC
CTGGACAGCTCCTCCTGGGGACCGGGGAAAGCGCTCTTGGGACCCAGGCC
GAATGCTCCCGTTTGGGCACAGCCTGGGGGCCGGGGCGCCCCAGACGCTC
CAGGGTCCCCGCCTGCCTTAAGCGTCTACCTCCTCCCCTTCACCCCACAA
CACGCGCTCTCTGGGCCCCCTTTCACCCACCCACCCCCAATTTCTATCTC
CTTGTAATCTGTGTGTCCTGCTTTAATGTTGGGGGGCACGGGCGCGAACT
CTGGGTCACTTTGACCCCACGCCCAGCACGTAGTAGGCGCCCAGCACTCG
TGTGGAGCGACTCTGGGAGGAAAACTAGGAAGGAGACCTGAGGAGCGCCC
CGCTCAGCTCCCTGGACAGACGTAGGACCCAGGAGCAGGGTTAATCCTCG
GCTGCTTTGTCACTGCGAAGTCTCCAGCGGAGTCTGTCAGCCAATCTGCA
TCATCCCCGGGAAGGATCTGACCCAACAAGACAAGGGAAAAGGAGCAGGG
CTGCTACTTCTTCAGCAGCTCTCCTCGGTTCGGTCTCCGAATAAGAAATA
ACAATTACATCACGAGGCCGGGGCGGCAGCGCGGTGGAGCCTCTCGGTTT
CCTGCAGCCTAATCTCACTGAACCCGCCTCTAAATTGGGGGGGCCACAGA
TTGGTGAAACGAGAAAGTGGAAGGAGGAGAGAGTCATACGAAACCATAAT
TTCCCCTTATCCTTGTATTTGCTGTTTGAAAAATCGTATCCTCAACGCTC
CCCAGACATCTCACTAGAACTATTAACACCCCCTCAACACCTCCCCAGAT
GTCAGGTTTCTTTTTGACTTGAGCATCTAGCATTTAGTAATTCGCCATGA
AAAAAATTACCAGGGGAAGCGAGTGTTGTTGCTGAAATTGTTTTTGCTTG
ACACAAAAAAGCTAAAGTACAGCTTAGTCTCACCCGAAATTCTGAAACGG
CATGCCCGTGTTAAAACACTTATTACATAAGACAAATGAGCCGATGAGGC
ACTCCATTCTTATATGTAGGTTTTCATATCCTGAAGAATTTGATAGCATT
TGGAAATGAAGTTGCTAATGCGCTTAAAAAACAAAAACTATCCAAGTCAT
GAAACTGTATTCTGTGCCTGTTATGTAATGGCTGTATTGTGTATTGCAAA
TTTCACTTAACCTTTAAACAATATGAATGAGAGTTTTAGAATACTGTCAC
AGTCTTGAAAGTGAAAGCAATTTTTCAAATTAATGCTTAATAAATTCAGC
ACCCAGCACAGTGCCGGGCACATAGTGCCAATGTTTTCCTTAGTGGCAAT
GGCACATTTTAAAACAATTATTTTTCATCACCCTAGTGATTGAACTATGC
AATCTGTTGCCATTTTATATAGTTAAACCTCATATAATATAATACCATAT
TATTTGCATATACCACAATTCAAAATCTGCTCTAGGCAAAATAAAGTCTG
CTGTTTTTTAGTGCATCATTTATTCAGAATTGATACAAAGCAAACCGTTT
CAATTCAATGATGACCAGCATAATTGGAGCTTGGCCCTTAGTGGGGGGTC
AAACTTTTTTTATGAAAATTATTTTTAACCATTATACAGATTTTTCTACA
A
Seq. ID. No: 114 NRIP1 promoter 2
CACAGTTGGTTCCTTTGTTCCTTCTGACTGAACACCAACTCCTGATCAAA
ATTGGGAAACTACTTTGGAAGGGATGGGAGGGTCTGCAGAATACATCCAC
GGAAACCTTCATAAAACAATATTCAATCGTAATAAGGTGGACACAGAAGA
AGGGAGAAAAGGCAGCCTCCCGGACTTCCACCGGGCTCACCCTAAATGGG
GGCCGGCCCCTGGCTCCTGGTAAAATGATGGCTGAGGCCTCGGGTCCGTG
AGCCTCGCCATCGGGAGGGGACCGCGAGTGCGGGCAGAGCGGCCCGGGGT
CGGGTGGGGGCCGGGCGAGCTGTCCCTTTTCCTATTCACTTTCCCAGCCG
GGTATCCAGCGAGCAAGGAGCAGGGAGGCACCCAGGCGAAAGGCGCCGCC
CAGGATCCGGCTGGACAGCAGGACGGAGAGCGGCGAGGCGAGCGGTGCCC
GGGGTTCGGGACCGAGAGGGCAAGCGAGCGGGGGCAACCCCGACCCGGCC
CCCCGAGTCCGGCCCCGCTCAGGGGCCACGCGACGGCCGCCCCCTTGCCC
AGAACTGGCACGGACGGGCGGGCCGACCCGGGGCCACTCCACAACTTGCC
GGCCCGCCGCCCCGGCCCCCCGCGAGAACCCGGAGACTCGAACCAGGCAG
CAGAGGCAGGATTTCCCCGGAAACGCGCCCTCCCCGCCGCCCCGACGCCC
CCGCGGCCCTGCCAGCCAAGCGAGAGCCGACGCGGGCCGCGCTCCCGGGC
CGTTTCCGGCGGCGCCGGCGGCCGCTACTCCTGGCGCCCTGGCCGCGGCC
GTCACTCACCCCAGGCCGCCGCGGCTCCCAGCCTCCGGCTCCGTCAGGCT
CGGTCCGCGAAGGCGCCTGCCGCCCCGTCCTGGCCCGGCGCCCCGGCGAG
CTCTTCCCTCCGACCAGCGGCGCTCACGGCGCAGCGGCGGACGCGAGGCC
ACGGGCGGACGGGCGCGCGCGGGTGGCGGGCGGGCGTGGGGCCGGGTCGT
CCCTGCGCCTCGCCGCCGCCTCCTCCGCCGCCGCCTCCCGAGTTCGGGGC
TCTGCTGCAATGTCTGCGAGGCTGACTTTGAGCGCCTCGCTCACCGCCCG
CCCGCAGCCGCGCTCCTCCCTCCGCCGCCGAGTCCGCCGCGGCCCAGTCC
CTCCCCTCGCCGCCCCACGCCATTCAGCTCTTCAGCCAAGTTTCTTAACC
CTTTTCTCCCCCTCCCTCCCATTGTCACCCGGTCAGGAGGAAGCCGCGCG
GATCCGGGCGGCTCACAGGAGCTGCGGAACCCCCTCTGTTTCGTGGATTT
CCTTCTCGCCTGCTCTCCGGGTGTCCCAGACGGGGCTGCAAGACCCGGAG
GCAGCGAGAGGACCGCGTCCCCGAGAGAGGGGCTGCACGGCGCGCCCGGG
CCGTCCCCCGGGCTCCCAGCTCTGTCCAGCCGCCCGCGACCGCTGCAGGC
GCCTGGACAGCTCCTCCTGGGGACCGGGGAAAGCGCTCTTGGGACCCAGG
CCGAATGCTCCCGTTTGGGCACAGCCTGGGGGCCGGGGCGCCCCAGACGC
TCCAGGGTCCCCGCCTGCCTTAAGCGTCTACCTCCTCCCCTTCACCCCAC
AACACGCGCTCTCTGGGCCCCCTTTCACCCACCCACCCCCAATTTCTATC
TCCTTGTAATCTGTGTGTCCTGCTTTAATGTTGGGGGGCACGGGCGCGAA
CTCTGGGTCACTTTGACCCCACGCCCAGCACGTAGTAGGCGCCCAGCACT
CGTGTGGAGCGACTCTGGGAGGAAAACTAGGAAGGAGACCTGAGGAGCGC
CCCGCTCAGCTCCCTGGACAGACGTAGGACCCAGGAGCAGGGTTAATCCT
CGGCTGCTTTGTCACTGCGAAGTCTCCAGCGGAGTCTGTCAGCCAATCTG
CATCATCCCCGGGAAGGATCTGACCCAACAAGACAAGGGAAAAGGAGCAG
GGCTGCTACTTCTTCAGCAGCTCTCCTCGGTTCGGTCTCCGAATAAGAAA
TAACAATTACATCACGAGGCCGGGGCGGCAGCGCGGTGGAGCCTCTCGGT
TTCCTGCAGCCTAATCTCACTGAACCCGCCTCTAAATTGGGGGGGCCACA
GATTGGTGAAACGAGAAAGTGGAAGGAGGAGAGAGTCATACGAAACCATA
ATTTCCCCTTATCCTTGTATTTGCTGTTTGAAAAATCGTATCCTCAACGC
TCCCCAGACATCTCACTAGAACTATTAACACCCCCTCAACACCTCCCCAG
ATGTCAGGTTTCTTTTTGACTTGAGCATCTAGCATTTAGTAATTCGCCAT
GAAAAAAATTACCAGGGGAAGCGAGTGTTGTTGCTGAAATTGTTTTTGCT
TGACACAAAAAAGCTAAAGTACAGCTTAGTCTCACCCGAAATTCTGAAAC
GGCATGCCCGTGTTAAAACACTTATTACATAAGACAAATGAGCCGATGAG
GCACTCCATTCTTATATGTAGGTTTTCATATCCTGAAGAATTTGATAGCA
TTTGGAAATGAAGTTGCTAATGCGCTTAAAAAACAAAAACTATCCAAGTC
ATGAAACTGTATTCTGTGCCTGTTATGTAATGGCTGTATTGTGTATTGCA
AATTTCACTTAACCTTTAAACAATATGAATGAGAGTTTTAGAATACTGTC
ACAGTCTTGAAAGTGAAAGCAATTTTTCAAATTAATGCTTAATAAATTCA
GCACCCAGCACAGTGCCGGGCACATAGTGCCAATGTTTTCCTTAGTGGCA
ATGGCACATTTTAAAACAATTATTTTTCATCACCCTAGTGATTGAACTAT
GCAATCTGTTGCCATTTTATATAGTTAAACCTCATATAATATAATACCAT
ATTATTTGCATATACCACAATTCAAAATCTGCTCTAGGCAAAATAAAGTC
TGCTGTTTTTTAGTGCATCATTTATTCAGAATTGATACAAAGCAAACCGT
TTCAATTCAATGATGACCAGCATAATTGGAGCTTGGCCCTTAGTGGGGGG
TCAAACTTTTTTTATGAAAATTATTTTTAACCATTATACAGATTTTTCTA
CAATGTATTGCAAATGTTTCTCTTAAGCAGTTTGTTGAACGTCATTTAGC
ATTTTCTTGATAATTCAATGTTAATTTTATTAAAAGTAATTATTGTAGTA
GGTTGTAGCGGATTCAGTAAATATATCCAGTTCCTATTCTATATTCTTTC
TAGGTGTTAGGAATGCTCCCAGGAGGTACAAAAGCTATCAGTCGCTGCAC
CGAAGGACTCTAGGATGCTATGACTTAGTGATTATCTCTGCCTTTTGTAA
TTTCTCTCTCTCCCCCTTCACTAACTGACAATCTATTGTCCTTACTGTTA
CTAGCATAGCTGCCACTAACATTCTCCTCATCTTCTGATGGGTAACTTCC
AGTCTAGTGCGGACTGGGAATCCAGATAGCCTCACTTGTCACAATTCTGT
CTAAAGTAAACTTAATAGGTTCTGGGTAAGGGACTAATGGGCTCACCAAG
GAGTAGGGCGCACACACACCTTAGTGCATAGACTTTGGGAAAGATATTTG
GACTACTCAAGTCTATCTTTTAAATACATTCCTCTTACTTCTTAACCAGG
TTTTGGACCATGGAGATTGCCCAGCAAGCCGAGAATATTCCAGCCCTCCA
ACATTGTTGTTAAAAAGTCCACTGCTGTATGTTAGGTGACTTCCCAAAGC
TGCTCACTACTTCTTCTTATTCTGTTTTCTCAACCCCACAAAAATCCAAT
CACTGAAATCCTGAACTCTTTTGATAGTGGCATGAGTGTTAAGTAAATTG
CACACCTCTTGTGGAATTCATTTTCACTTAATCATGCTAATTATGTGGAG
ACTTGTTTGTTGTTGTTCTTTGTTTTTTTAAGTAAATCAATACCTAAATT
TCCTGGCTAACAGAGTCATTGCAAGGATACTAGGGCATTATATGTAATAT
AAGAATGCCTATTTGGTAATTTGTACTTTGAAGTCAAGTCTCATTTGAAT
C
Seq. ID. No: 115 UPP1
MAATGANAEKAESHNDCPVRLLNPNIAKMKEDILYHFNLTTSRHNFPALFGDVKFV
CVGGSPSRMKAFIRCVGAELGLDCPGRDYPNICAGTDRYAMYKVGPVLSVSHGMGI
PSISIMLHELIKLLYYARCSNVTIIRIGTSGG
Seq. ID. No: 116 UPP1 promoter
TGCAGTAAAGAAGCCAGCCAAAACTGGCCAAAACCTGCCAAAAACCAGCA
AGGTGAGGAAAACTACCTCTGGTCTTCCTCATTGCTCATTATTCGCTAAT
TACAATGCATTCTCATGCTAAACGACACTCCTACCAGCACCATGACAGTT
TATAAATACCATGGCAACATTTAGAAGTTACCCTATATGTTCTAAAAGGA
AGAATTCCTAGAAAACTCATGAATAATCCACCCTTATTTAGTATATGATC
AAGAAATAACCATAAAAATAGCCAACCAGCAGCCCTAGAGGCTGCCCTGC
CCATGGAATTGCCATTCTTTTATTCCTTTACTTTCTCAATAAGCTTGCTT
CTTTCACTTTACTCTGTGGACTCACCCTGGCCCCGAATTCTTTCTTGCAC
AAGATCCAAGAACATTCTCTTGGGGTCTGGATTGGGACCCTTTTCCGGTA
ACAAAACTAAAACTAAGTTGTTAATCTACCTTCAGAGAGAGAGAATAGCA
ACTTCCTAACCAGGCCAGTTTCAGTCTTCAGTCTGAGGCAGCAAAATAGG
GTCTGGAGGCAGGGAACATAAGGCCGATTCACACTTTAGCTATGACAGGA
AATAGCCTCTGCGTAGGCCAAGTAAATGACTTTGTAACTTTGCATCATCC
CTTTCATTTACATAGTGTGTGCCCCAAGCGGAGGACATTGAAACTCCCCA
AAATTCTGGAATGGGGCCTTTGAGCCCCTACGCTCAGGCCCGCTCCCACA
CTGTGGAGTGTACTTTCATTTACCTTTTTTTTTTGAGATGGCGTCTCACT
GTGTCACCAAGGCTGGAATGCAGTGGCATGATCTCAGCTCACTGCAACCT
CTGCCACCCAGGTTCAAGTGGTTCTCCTGCCTCAGCCTCCCAAGTAGCTG
GAATTACAGGCACCTGCCACTGTGACCGGCTAGGTTTTTTTTTTTGTTTT
TTTTTTTTTTTTACTTTTAGTAGAGACGGGGTTTCACCATCTTGGTTAGG
CTGGTCTTGAACTCCTGACTTTGTAATCCACCCGCCTCGGCCTCCCAAAGGGTTTC
ACCATGTTGGGCAGGCTGGTCTCGAACTCCTGACCTCAGGTGAT
CCATCCGCCTCGGCCTTCCAAAGTGCTGGGATTACAGGCGTGAGCCACCA
CTCCCGGCCCAACCCCATGTTTTCTCAGGAACTGGAGTTCCCTTTAGGAA
GCAAGTAACAGAGGTGCTGGGCTGCCCGGGCCTCTAGGTCGTCTTCTTTC
CCAGGCTCTGTGCTCTCACCAGAGTGCGCGTTCGGGAAGCAGGGAGAGGA
CCAGGAAAGACGCCTGAGACGGAGAGTGGGCACCGGCCGGGGGCAGGCAG
GTCCGGCCCCTGGGGGTCAGAGTGCAGCGCCGGTTTGAGGCAGTTCGGCC
ACTTAGCTGCGACTCGAAGTGAATGAGTCACTTCCCCTCCTCGGGCGAGC
TCCTGGCTTTGCCCCAGCAAGCGGGAACTCCCAGGATCAGGAAGGTCCCA
GAGAAACTCCCCAGGGGGGAAGCCGAGGCCGTCCAGAGAGATCCAGGGTA
CACACAGTGACAGTGCCAGCTCCACATTCCCGACCACCGGGCCAGGGCTG
GTTCCACGCTGTCATTTCTCTCCGCTGGCCCCAGCCGCGGGGCCACCGCC
AGACAGACAGACCGGGGATCCTGCCTGGCGGCGTGACCTTGGGTTAGTCC
TAGAGCCGCTTCTAGCCCGGTTTCCTCCGCCCCAGGTTAGGACCACGTGG
GTAAGAAGATTTCCTCTGTAGTAGGGGCTGTTTAATGAGTAACAGAACTG
GCTGCTGGTTCTTTACAATTGCCCAGCTGCTCTGCAGAGGCGAGAAGCAA
TTGCGGGCTGAGCCTCCCGGGACCTTCGCGGGAGGGTGGAGAGGAGGAAG
GCGGGGGAGGCGGGGCCAGAGGGGCAGAGGAAGGTGGGGGCGGGCGGGGC
TTGTCTGCGGGATGGCCCAGGATGCGGTCAGCTGAGTTCGCCGGCCCAGG
GCAGGCGGGGCCCGAGCCTAGCGGTAACCCCCGGGCAGGGCGGGGCCGCT
CGCAGACTCCATATGAGATTCACCTCGCAGGTGGTTCCCTCATTCGAGTG
CTCCGGCGCACAGACCCGCGCCCCGCCGTCTGCGAGCCTCCCGAGAGCCG
TCCCTTCGTCCGGCCCTGGAGCATTGCGTTTGTCGCCGGTGTCGCAGTGC
GAGGATGGCGCCGCGGGTGTAGCGGCTCTCTGCGCAGGCCGAGTGGTGAG
TCGCCCGCGGCCCGGCTCCCGCCGTTCTCTCTTCCCAGGGGTCTAGCCCG
TCCCCTGCCTTGGCCACGTGCCCATTTACCTGTAGGGGCGGTGACGGAGG
CTCTGGTGCACCTTGGCTGTGAGCTGCAGAGGAGCAGAGAGCGGCCCGGC
GCGGCTGCGCGGGGAGGCGGGCGCGCGGCCGGGAGGTTCTGGGGCTGGTG
TGTGCGGAGCGAGGTGGGCGTGGGGGAAGCGCGCGGGCGGGATGTGCTTC
CTCGGGTCCACCCCGTGCCTACCTCTCCCACGTCCCGGCGACGGAGCGCG
CGGGGCTCTGGGCGGGGAGCCCACCGGGCCGAGGCGCAGCGAGGGTGAAG
CCAGAGCCGGCAGGGTCAGGTCCCGCAGGGCGCAGGCGGGCGCTTCTCGC
AGCCCAAACCCGGGCGGGGAGTGCGGGAACCCCTAGAACTATGCTGGGAG
CCCCAGATAAGAGGAAGGAGATGGTGACTTAAGTGACGAAGTGATGTTTT
GTCCCCACCGCCTTTTAAAGGGAGAAGGGGGTAAAATTTCATTTGGAGGA
TGATTGTTACGCGAGCTTTCAGAGTTCAGATGGCGTCAAGTGGCCATCGA
GTCAACATTTAACAAAATGTTACCGCATAAAATATTGTCATCGAGTTATC
TGGCAGCTCCGCAGAGAAACTTCACAGGCATATGCGTTATATGACATAAT
TTGCATGGGGAAGGGGGTCCCTTCACGATTCTTTTATATGGCAACCTGTT
CTTGGACCTGGCAGGG
SEQ ID NO: 117 CKB protein sequence
MPFSNSHNALKLRFPAEDEFPDLSAHNNHMAKVLTPELYAELRAKSTPSGFTLDDVI
QTGVDNPGHPYIMTVGCVAGDEESYEVFKDLFDPIIEDRHGGYKPSDEHKTDLNPDN
LQGGDDLDPNYVLSSRVRTGRSIRGFCLPPHCSRGERRAIEKLAVEALSSLDGDLAGR
YYALKSMTEAEQQQLIDDHFLFDKPVSPLLLASGMARDWPDARGIWHNDNKTFLV
WVNEEDHLRVISMQKGGNMKEVFTRFCTGLTQIETLFKSKDYEFMWNPHLGYILTC
PSNLGTGLRAGVHIKLPNLGKHEKFSEVLKRLRLQKRGTGGVDTAAVGGVFDVSNA
DRLGFSEVELVQMVVDGVKLLIEMEQRLEQGQAIDDLMPAQK
SEQ ID NO: 118 CKB Promoter
AAGACCCCGGCCCCGCCCGCCCGGCCCGCCCGCAGCCCCGCACCTGCAGG
TTGTCGGGGTTGAGGTCGGTCTTGTGCTCATCGCTGGGCTTGTAGCCGCC
GTGCCGGTCCTCGATGATGGGGTCGAAGAGATCCTTGAACACTTCGTAGG
ACTCCTCGTCGCCCGCCACGCAGCCCACGGTCATGATGTACGGGTGGCCT
GGGGGAGGGGGCGCGGGACGGGGACAGTGACGTCACTGCCGGGCCCGAGC
GCGCTGCTGAGGACCCTGCGGCTGCGCGGGGGGAGGGGGGGCCGGGACCC
CGGCCCCGAGGGGTCGCGTACCCGGGTTGTCCACGCCTGTCTGGATGACG
TCGTCCAGCGTGAAGCCGCTCGGCGTGCTCTTGGCGCGCAGCTCCGCGTA
CAGCTCGGGGGTCAGCACCTTGGCCATGTGGTTGTTGTGGGCGCTCAGGT
CGGGGAACTCGTCCTCGGCCGGGAAGCGCAGCTTCAGTGCGTTGTGGCTG
TTGGAGAAGGGCATGGCGGCGGCGGGCTGCGGGGAGACGCGGGGTCAGAG
GGGACCGGCACGCCGGGGTTCCCGGGCTCCCGCGTACCACTCAGGCCCCC
GCCGCCGGGCCCCCCGGCGCCCCCCGGGACGCGGCCAAGGTCAGCGGGGT
CCGCAGCGCGCGCGGGGAGCGCCATCATCGCCGGGGACCCCCGGCCGGTC
CGGCGCGCGCTCCAGGCGACGCGCCCCCTTTGCACGCAGCGCCCCTAGCC
CGGGCGCGGACGCCCCCGGCCCCCGGGCCCACTCACCGGGCGGCCGGGCG
GGGGCGGGGGCGCTCCGTCCGTCGGCAGCTCCCGGAGCGACGCAGGCGAA
CAGCGGCCGCTCCGCGCTCCCGCGGCTCTTAAGGGGCACAACGCGGGCCG
CCCATTGGCGGCTATTTATAGCCCATTCATTCCATTGGCCCGCGCCGCGG
GGTGGTGCCGCCGCTTTGTCCGCCGCCCGCAGCCCCCAACACCCTGGCCG
CCCACCCGAGCTGTCCCCTCGCTGGGACCCCGCGCAGCCTGCCCAGTCCC
CCGCCCAAGACCTCGAGGCCGAAACCTCAGGTCCTTGGATATCTGGATCC
CCTCGCACGCCCTTGCCTCTGGGGCGGAAACTGAGGCGGAGAGCCTCCGG
GCACTCGAGGACGCTTGGGTTCATCCGCATCCTCGAGCCTGCCCGCCTAG
TCCAGCCAGCCCCCGCGGCGGCGGCGAGGGGCCGGGCGAAAGCTTTCGGG
GCTTCCGCAGCAGGTACGGCCCCCGCCCCACCCCCGGCCAGTGCGAGCTG
GCCCGGAGCCGCCGACCTTCCTCAAGGTGCCCGCAGGCGTTTCCTGGGCG
ATCCTGGTGCAAAGCGAAGGCGCGCGGGAAGCGCAGTCTGGCTGCGGCCC
AGCCCGGGGGCACCCGCGGTGCCCTGACCTTGGACAAGTCCTGGGTCTCG
GGGGGCTGCGAGCCAGAAACCACGGGGACGCTGTGGCCTTAAGAGGCCGC
TGGCAAGTGCAGCCTCGGAGTTTCCAGACCCCAACCCTGGGCCCTCTTCG
CAGGGGACAAGTTTACCCGACCCCCAAAGGCAGTCCCCGGAGACTTCAGG
CAAAGATGGGAGGAAACGCTAATAAACAATAAGCATAAGTGCGATTAAAA
AAAAAAACTGATCTCACGAATAACCAGAATCATGCAAATTAAGCAGTGAC
TCCACCTGCCCCTACCCCCAACACTCCCAGATCGACCCCAGCCATTGCTG
GCTTTGAGCTGGAATGCGGGAAGACAGGCGTCCACACGCTGCCTGCAACT
TAAATTGCTGCATGC

1-50. (canceled)

51. A method for preventing or treating a metabolic disease in a subject, comprising:
upregulating or overexpressing at least one polypeptide selected from the group consisting of uncoupling protein 1 (UCP1/SLC25A7), PPARG coactivator 1 alpha (PPARGC1A/PCG1A), PR/SET domain 16 (PRDM16), FFAR1/GPR40, FFAR4/GPR120, FOXO1, GLUT1, GLUT2, GLUT3, GLUT4, CHREBP, CD36, CTP1B, AIFM2/FSP1, GSK, IRS1, DGAT1, DGAT2, uridine phosphorylase (UPP1), CKB, and ACACA or as found in Table 2, or downregulating ACACB or NRIP1, in an adipocyte of the subject, thereby preventing or treating a metabolic disease.

52. The method of claim 51, wherein upregulating the polypeptide comprises introducing to the subject a therapeutically effective amount of a composition comprising a targeted polypeptide linked to a transcriptional activator, wherein the targeted polypeptide targets a portion of a promoter or enhancer sequence operably linked to a polynucleotide encoding the polypeptide.

53. The method of claim 52, wherein the targeted polypeptide is a catalytically inactive nuclease, wherein the catalytically inactive nuclease is (i) a clustered regularly interspaced short palindromic repeats (CRISPR) nuclease, and the engineered adipocyte or adipose stem cell further comprises a gRNA that targets the portion of the promoter or enhancer sequence; (ii) a zinc finger nuclease (ZFN); or (iii) a transcription activator-like effector nuclease (TALEN).

54. The method of claim 53, wherein the gRNA comprises a sequence having at least 90%, 95%, 98%, 99% or 100% identity to a sequence of any one of SEQ ID Nos: 1-55.

55. The method of claim 52, wherein the targeted polypeptide is a catalytically-inactive nuclease dCas9.

56. The method of claim 52, wherein the transcriptional activator is selected from the group consisting of HSF1, VP16, VP48, VP64, VP160, p53, p65, RTA, MyoD1, SET7, LSD1, CIB1, AD2, CR3, GATA4, SP1, MEF2C, TAX, SET9, VP64-p65-RTA (VPR), Histone Acetyltransferase p300, TET1 Hydroxylase Catalytic Domain, Synergistic activation mediator (SAM), SunTag, and PPAR-gamma.

57. The method of claim 52, wherein the transcriptional activator is VP64.

58. The method of claim 52, wherein the transcriptional activator is VP64, and the targeted polypeptide is catalytically-inactive nuclease dCas9.

59. The method of claim 51, wherein overexpressing the polypeptide comprises introducing to the subject a therapeutically effective amount of a composition comprising a heterologous expression cassette comprising a promoter operably linked to a coding sequence encoding the polypeptide.

60. The method of claim 51, wherein downregulating the polypeptide comprises introducing to the subject a therapeutically effective amount of a composition comprising
(i) a targeted polypeptide linked to a transcriptional repressor, wherein the targeted polypeptide targets a portion of a promoter or enhancer sequence operably linked to at least one polynucleotide encoding a polypeptide selected from the group consisting of ACACB and NRIP1, or
(ii) an siRNA or antisense molecule that comprises a sequence complementary to ACACB and NRIP1.

61. A method for preventing or treating a metabolic disease or a symptom of aging in a subject, comprising:
introducing to the subject a therapeutically effective amount of engineered adipocytes, engineered adipose stem cells, or engineered adipose organoids, wherein the engineered adipocytes, engineered adipose stem cells, or engineered adipose organoids upregulate or overexpress at least one polypeptide selected from the group consisting of Uncoupling protein 1 (UCP1/SLC25A7), PPARG coactivator 1 alpha (PPARGC1A/PCG1A), PR/SET domain 16 (PRDM16), FFAR1/GPR40, FFAR4/GPR120, FOXO1, GLUT1, GLUT2, GLUT3, GLUT4, CHREBP, CD36, CTP1B, AIFM2/FSP1, GSK, IRS1, DGAT1, DGAT2, UPP1, CKB, and ACACA or as found in Table 2, or downregulate expression of ACACB or NRIP1.

62. The method of claim 61, wherein before the introducing, isolating adipocytes or adipose stem cells from the subject and engineering the cells to have the upregulated or downregulated expression or the overexpression.

63. The method of claim 62, further comprising: culturing the engineered cells into an engineered adipose organoid, and
introducing the engineered adipose organoid to the subject.

64. The method of claim 61, wherein the introducing engineered adipocytes, engineered adipose stem cells, or the engineered adipose organoid are autologous.

65. The method of claim 61, wherein the introducing engineered adipocytes, engineered adipose stem cells, or the engineered adipose organoid are allogeneic.

66. The method of claim 61, wherein the introducing comprises introducing the engineered adipocytes, engineered adipose stem cells, or the engineered adipose organoid intravenously, intramuscularly, or a combination thereof.

67. A method of treating or preventing cancer in an individual, the method comprising introducing into an individual one or more engineered adipocytes or adipose stem cells in proximity to cancer cells, wherein the one or more engineered adipocytes or adipose stem cells consume a metabolite preferentially consumed by the cancer cells compared to non-cancer cells in the individual, thereby inhibiting growth of the cancer cells.

68. The method of claim 67, wherein the cancer cells are pancreatic ductal adenocarcinoma cells, and wherein the engineered adipocytes or adipose stem cells are introduced into the breast of the individual.

69. The method of claim 67, wherein the engineered adipocytes or adipose stem cells upregulate or overexpress at least one polypeptide selected from the group consisting of Uncoupling protein 1 (UCP1/SLC25A7), PPARG coactivator 1 alpha (PPARGC1A/PCG1A), PR/SET domain 16 (PRDM16), FFAR1/GPR40, FFAR4/GPR120, FOXO1, GLUT1, GLUT2, GLUT3, GLUT4, CHREBP, CD36, CTP1B, AIFM2/FSP1, GSK, IRS1, DGAT1, DGAT2, uridine phosphorylase (UPP1), CKB, and ACACA or as found in Table 2, or downregulate expression of ACACB or NRIP1.

70. The method of claim 69, wherein the engineered adipocytes or adipose stem cells upregulate or overexpress UPP1.
